(19) United States
(12) Patent Application Publication
(10) Pub. No.: US 2025/0222088 A1
(43) Pub. Date: Jul. 10, 2025

(54) MULTIVALENT VACCINE COMPOSITIONS AND USES THEREOF

(71) Applicant: Janssen Pharmaceuticals, Inc., Titusville, NJ (US)

(72) Inventors: Jan Theunis POOLMAN, Vogelenzang (NL); Kellen Cristhina FAE, Oegstgeest (NL); Michal SARNECKI, Basel (CH); Jeroen GEURTSEN, Vleuten (NL); Darren Robert ABBANAT, Cornwall, NY (US); Bart Gustaaf M. SPIESSENS, Bierbeek (BE); Frank Germaine F. STRUYF, Bierbeek (BE)

(21) Appl. No.: 19/025,061

(22) Filed: Jan. 16, 2025

Related U.S. Application Data
(62) Division of application No. 17/478,584, filed on Sep. 17, 2021, now Pat. No. 12,233,118.
(60) Provisional application No. 63/079,734, filed on Sep. 17, 2020.
(60) Provisional application No. 63/191,471, filed on May 21, 2021.

(30) Foreign Application Priority Data
Feb. 2, 2021 (EP) 21154782.3

(51) Int. Cl. A61K 39/108; A61K 31/739; A61K 39/00; A61K 47/64

(52) U.S. Cl. CPC A61K 39/0258; A61K 31/739; A61K 47/6415; A61K 47/646; A61K 2039/55583; A61K 2039/70

(57) ABSTRACT

Compositions and methods are described for inducing an immune response against extra-intestinal pathogenic Escherichia coli (ExPEC) to thereby provide immune protection against diseases associated with ExPEC. In particular, compositions and methods are described for using conjugates of E. coli polysaccharide antigen O75 covalently bound to a carrier protein for the prevention of invasive ExPEC disease.

### CROSS-REFERENCE TO RELATED APPLICATIONS

This application is a divisional application of U.S. application Ser. No. 17/478,584 filed Sep. 17, 2021, which claims priority to provisional U.S. Application No. 63/079,734 filed Sep. 17, 2020, provisional application U.S. Application No. 63/191,471 filed May 21, 2021, and European Application No. 21154782.3 filed Feb. 2, 2021, the disclosures of which are incorporated herein by reference in their entireties.

### REFERENCE TO SEQUENCE LISTING SUBMITTED ELECTRONICALLY

The contents of the electronic sequence listing (004852.168US4.xml; Size: 164,740 bytes; and Date of Creation: Jan. 14, 2025) is herein incorporated by reference in its entirety.

### BACKGROUND OF THE INVENTION

Extraintestinal pathogenic Escherichia coli (ExPEC) are normally harmless inhabitants of human gut, alongside commensal E. coli strains. However, ExPEC strains can possess virulence factors for the colonization and infection of sites outside of the gastrointestinal tract to cause diverse and serious invasive diseases, resulting in significant morbidity, mortality, and costs annually (see, e.g., Johnson et al., J Lab Clin Med. 2002; 139(3):155-162; Kohler et al., Int J Med Microbiol. 2011; 301(8):642-647; Foxman, Am J Med. 2002; 113 Suppl 1A:5S-13S; and Russo et al., Microbes Infect. 2003; 5(5):449-456). ExPEC strains are the most common cause of urinary tract infection (UTI). They are also a contributor to surgical site infections and neonatal meningitis (Johnson et al., 2002; and Russo et al., 2003), associated with abdominal and pelvic infections and nosocomial pneumonia, and are occasionally involved in other extra-intestinal infections such as osteomyelitis, cellulitis, and wound infections. All these primary sites of infection can result in ExPEC bacteremia (Russo et al., 2003). Neonates, the elderly, and immunocompromised patients are particularly susceptible to ExPEC infection, including invasive ExPEC disease (IED).

Bacterial resistance to antibiotics is a major concern in the fight against bacterial infection, and multi-drug resistant (MDR) E. coli strains are becoming more and more prevalent (see, e.g., Schito et al., 2009, Int. J. Antimicrob. Agents 34(5):407-413; and Pitout et al., 2012, Expert Rev. Anti. Infect. Ther. 10(10):1165-1176). The emergence and rapid global dissemination of ExPEC sequence type 131 (ST131) is considered the main driver of increased drug resistance, including multi-drug resistance (Johnson et al., Antimicrob Agents Chemother. 2010; 54(1):546-550; Rogers et al., J Antimicrob Chemother. 2011; 66(1):1-14). This clone is found in 12.5% to 30% of all ExPEC clinical isolates, mostly exhibits serotype O25B:H4, and shows high levels of fluoroquinolone resistance, which is often accompanied by trimethoprim/sulfamethoxazole resistance and extended-spectrum beta-lactamases conferring resistance to cephalosporins (Rogers et al, 2011, and Banerjee et al., Antimicrob Agents Chemother. 2014; 58(9):4997-5004).

The O-antigen serotype is based on the chemical structure of the 0 polysaccharide antigen, the outer membrane portion of the lipopolysaccharide (LPS) in a Gram-negative bacterium. More than 180 serologically unique E. coli O-antigens have been reported (Stenutz et al., FEMS Microbial Rev. 2006; 30: 382-403), although the vast majority of ExPEC isolates are classified within less than 20 O-antigen serotypes. Full-length E. coli O-antigens are typically comprised of about 10 to 25 repeating sugar units attached to the highly conserved LPS core structure, with each component synthesized separately by enzymes encoded predominantly in the rfb and rfa gene clusters, respectively. Following polymerization of the O-antigen, the O-antigen polysaccharide backbone may be modified, typically through the addition of acetyl or glucose residues. These modifications effectively increase serotype diversity by creating antigenically distinct serotypes that share a common polysaccharide backbone, but differ in side branches. Genes encoding O-antigen modifying enzymes typically reside outside of the rfb cluster on the chromosome, and in some cases, these genes are found within lysogenic bacteriophages.

ExPEC infection can be caused by any serotype. Although there is an overrepresentation of certain serotypes in ExPEC infection, surface polysaccharides from ExPEC isolates nonetheless exhibit considerable antigenic diversity, which makes the development of an ExPEC vaccine based on surface polysaccharides extremely challenging (Russo et al., Vaccine. 2007; 25: 3859-3870). Also, certain O-antigens may be poorly immunogenic. Furthermore, based on studies from Pneumococcal conjugate vaccines, when a number of serotypes can cause a disease, the vaccine composition, such as the choice of serotypes for inclusion in a vaccine and the dosage levels of the included serotypes, can be critical, since use of a vaccine against certain serotypes may potentially increase carriage of and disease from serotypes not included in the vaccine, or even a serotype that is included in the vaccine but only weakly effective in immunizing against the serotype (Lipsitch, Emerging Infectious Diseases; 1999, 5:336-345). Ideally, a vaccine should maximize its beneficial effects in the prevention of disease caused by serotypes included in the vaccine, while minimizing the risk of added disease from increased carriage of non-vaccine serotypes.

Efforts toward the development of a vaccine to prevent ExPEC infections have focused on O-antigen polysaccharide conjugates. A 12-valent O-antigen conjugate vaccine was synthesized through extraction and purification of O-antigen polysaccharide and chemical conjugation to detoxified Pseudomonas aeruginosa exotoxin A and tested for safety and immunogenicity in a Phase 1 clinical study (Cross et al., J. Infect. Dis. (1994) v. 170, pp. 834-40). This candidate vaccine was never licensed for clinical use. A bioconjugation system in E. coli has been developed recently, in which the polysaccharide antigen and the carrier protein are both synthesized in vivo and subsequently conjugated in vivo through the activities of the oligosaccharyl transferase PglB, a Campylobacter jejuni enzyme, expressed in E. coli (Wacker et al., Proc. Nat. Acad. Sci. (2006) v. 103, pp. 7088-93). This N-linked protein glycosylation system is capable of the transfer of diverse polysaccharides to a carrier protein, allowing for straightforward methods to purify the conjugate.

Bioconjugation has been used successfully to produce conjugate polysaccharide for an E. coli four-valent O-antigen candidate vaccine (Poolman and Wacker, J. Infect. Dis. (2016) v. 213(1), pp. 6-13). However, the development of a successful ExPEC vaccine requires coverage of predominant serotypes, and the presence of further O-antigen modifications in subsets of ExPEC isolates presents a further challenge in covering isolates displaying unmodified and modified LPS. Moreover, immune responses to vaccine compositions comprising O-antigens from multiple serotypes may differ between the serotypes. Accordingly, there is a continued need in the art for vaccines against ExPEC. In particular, there exists a need for an ExPEC vaccine based on surface polysaccharides that can be used to provide effective immune protection against ExPEC O75 serotype and other serotypes prevalent among ExPEC.

### BRIEF SUMMARY OF THE INVENTION

It has been surprisingly discovered that E. coli O75 antigen appears less immunogenic than other E. coli O-antigens (e.g., O1, O2, and O6) when tested as conjugates of the O-antigens each covalently bound to a carrier protein in a composition with such other O-antigens at the same concentrations in a clinical trial. Vaccination with a composition containing conjugates of E. coli O75 antigen and conjugates of one or more additional E. coli O-antigens at an appropriate dose and ratio provides an improved immune response against the ExPEC O75 serotype and the one or more additional ExPEC O-serotypes.

Accordingly, in a first general aspect provided herein is a composition comprising E. coli O1, O2, O4, O15, O16, O18, O25, O75 and O6 antigen polysaccharides, wherein each of the antigen polysaccharides is independently covalently linked to a carrier protein, and wherein the concentration of each of O75 and O25 antigen polysaccharides is independently increased relative to the concentration of each of O1, O2, O4, O15, O16, O18 and O6 antigen polysaccharides.

In certain embodiments, the weight ratio of concentrations of O75 antigen polysaccharide independently to each of O1, O2, O4, O15, O16, O18 and O6 antigen polysaccharides is about 1.5:1 to about 2.5:1.

In certain embodiments, the weight ratio of concentrations of O75 antigen polysaccharide independently to each of O1, O2, O4, O15, O16, O18 and O6 antigen polysaccharides is about 2:1.

In a second general aspect, provided herein is a composition comprising E. coli O1, O2, O4, O15, O16, O18, O25, O75 and O6 antigen polysaccharides, wherein each of the antigen polysaccharides is independently covalently linked to a carrier protein, and wherein the weight ratio of concentrations of O75 antigen polysaccharide to O1, O2, and/or O6 antigen polysaccharide is about 1.5:1 to about 4:1. Preferably wherein the weight ratio of concentrations of O75 antigen polysaccharide to O1, O2, and/or O6 is about 2:1.

In certain embodiments, the weight ratio of concentrations of O75 antigen polysaccharide to O4, O15, O16 and/or O18 antigen polysaccharide is about 1.5:1 to about 4:1. Preferably the weight ratio of concentrations of O75 antigen polysaccharide to O4, O15, O16 and/or O18 antigen polysaccharide is about 2:1.

In a third general aspect, provided herein is a composition comprising E. coli O1, O2, O4, O15, O16, O18, O25, O75 and O6 antigen polysaccharides, wherein each of the antigen polysaccharides is independently covalently linked to a carrier protein, and wherein the concentration of each of O75 and O25 antigen polysaccharides is independently increased relative to the concentration of each of O1, O2, O4, O15, O16, O18 and O6 antigen polysaccharides, wherein the weight ratio of concentrations of O75 antigen polysaccharide to O1, O2, and/or O6 antigen polysaccharide is about 1.5:1 to about 4:1. Preferably, preferably the weight ratio of concentrations of O75 antigen polysaccharide to O1, O2, and/or O6 antigen polysaccharide is about 2:1.

Certain embodiments of the first, second and third general aspect are now described.

In certain embodiments, the weight ratio of concentrations of O75 antigen polysaccharide to O25 antigen polysaccharide in the composition is about 1:1.

In certain embodiments, the weight ratio of concentrations of the E. coli antigen polysaccharides O1:O2:O4:O6:O15:O16:O18:O25:O75 is 1:1:1:1:1:1:1:2:2.

In certain embodiments, the O1 antigen is O1A, the O4 is glucosylated, the O6 antigen is O6A, the O18 antigen is O18A, and the O25 antigen is O25B, wherein:
(i) the E. coli O1 antigen polysaccharide comprises the structure of Formula (O1A) shown in Table 1,
(ii) the E. coli O2 antigen polysaccharide comprises the structure of Formula (O2): shown in Table 1,
(iii) the E. coli O4 antigen polysaccharide comprises the structure of Formula (O4-Glc+) shown in Table 1,
(iv) the E. coli O6 antigen polysaccharide comprises the structure of Formula (O6A) shown in Table 1,
(v) the E. coli O15 antigen polysaccharide comprises the structure of Formula (O15) shown in Table 1,
(vi) the E. coli O16 antigen polysaccharide comprises the structure of Formula (O16) shown in Table 1,
(vii) the E. coli O18 antigen polysaccharide comprises the structure of Formula (O18A) shown in Table 1,
(viii) the E. coli O25 antigen polysaccharide comprises the structure of Formula (O25B) shown in Table 1, and
(ix) the E. coli O75 antigen polysaccharide comprises the structure of Formula (O75) shown in Table 1,
wherein each n is independently an integer of 1 to 100, preferably of 3 to 50, for example 5 to 40, preferably of 5 to 30, for example 7 to 25, for example 10 to 20.

In certain embodiments, the concentration of the O75 antigen polysaccharide is from about 8 to about 64 μg/mL, preferably about 8 to about 50 μg/mL, preferably about 12 to about 40 μg/mL, preferably about 16 to about 32 μg/mL, preferably about 28 to about 36 μg/mL, preferably about 32 μg/mL.

In certain embodiments, the E. coli O antigen polysaccharides present in the composition consist of O1, O2, O4, O15, O16, O18, O25, O75 and O6.

In certain embodiments, the composition further comprises at least one additional E. coli antigen polysaccharide covalently linked to a carrier protein, preferably wherein the at least one additional E. coli antigen polysaccharide comprises O8 antigen polysaccharide with Formula (O8) shown in Table 1, wherein n is an integer of 1 to 100, preferably of 3 to 50, for example 5 to 40, preferably of 5 to 30, for example 7 to 25, for example 10 to 20.

In certain embodiments, the carrier protein is detoxified exotoxin A of Pseudomonas aeruginosa (EPA) or CRM₁₉₇, preferably EPA. Preferably the carrier protein comprises 1 to 20, such as 1 to 10, or 2 to 4, glycosylation consensus sequences having the amino acid sequence of SEQ ID NO: 1, such as the consensus sequences having the amino acid sequence of SEQ ID NO: 2. More preferably the carrier protein comprises four of the glycosylation consensus sequences. Most preferably each carrier protein is EPA comprising the amino acid sequence of SEQ ID NO: 3.

In certain embodiments, the E. coli antigen polysaccharides are covalently linked to the carrier protein by bioconjugation or by chemical conjugation, preferably the E. coli antigen polysaccharides are covalently linked to the carrier protein by bioconjugation, preferably the polysaccharide is covalently linked to an Asn residue in a glycosylation site in the carrier protein.

In another aspect, there is provided a method of inducing an immune response to E. coli, preferably extra-intestinal pathogenic E. coli (ExPEC), in a subject, comprising administering to the subject the composition of the invention.

In a further aspect there is provided a method of inducing an immune response to E. coli, preferably extra-intestinal pathogenic E. coli (ExPEC), in a subject, comprising administering to the subject an effective amount of each of E. coli O1, O2, O4, O15, O16, O18, O25, O75 and O6 antigen polysaccharides, wherein each of the antigen polysaccharides is independently covalently linked to a carrier protein, wherein the effective amount of each of O75 and O25 antigen polysaccharides is independently increased relative to each of O1, O2, O4, O15, O16, O18 and O6 antigen polysaccharides.

In certain embodiments according to a method of the invention, the effective amount of O75 antigen polysaccharide is administered at a weight ratio of independently about 1.5:1 to about 2.5:1, preferably about 2:1, to each of O1, O2, O4, O15, O16, O18 and O6 antigen polysaccharides.

In certain embodiments according to a method of the invention, the effective amount of O75 antigen polysaccharide is administered at a weight ratio of independently about 1.5:1 to about 4:1, preferably about 2:1, to O1, O2, and/or O6 antigen polysaccharide, preferably further wherein the effective amount of O75 antigen polysaccharide is administered at a weight ratio of about 1:1 to O25 antigen polysaccharide.

In another aspect, there is provided a method of inducing an immune response to E. coli, preferably extra-intestinal pathogenic E. coli (ExPEC), in a subject, comprising administering to the subject an effective amount of each of E. coli O1, O2, O4, O15, O16, O18, O25, O75 and O6 antigen polysaccharides, wherein each of the antigen polysaccharides is independently covalently linked to a carrier protein, the effective amount of O75 antigen polysaccharide is administered at a weight ratio of independently about 1.5:1 to about 4:1, to O1, O2, and/or O6 antigen polysaccharide. The effective amount of O75 antigen polysaccharide may preferably be administered at a weight ratio of about 1:1 to O25 antigen polysaccharide. Preferably, the effective amount of O75 antigen polysaccharide is administered at a weight ratio of independently about 2:1, to O1, O2, and/or O6 antigen polysaccharide.

Provided below are embodiments of any method aspect of the invention.

In certain embodiments according to a method of the invention, the immune response limits the severity of or prevents an invasive ExPEC disease in the subject, preferably wherein the invasive ExPEC disease comprises sepsis and/or bacteremia.

In certain embodiments according to a method of the invention, the O1 antigen is O1A, the O4 is glucosylated, the O6 antigen is O6A, the O18 antigen is O18A, and the O25 antigen is O25B. Preferably the O1A, O2, glucosylated O4, O6A, O15, O16, O18A, O25B, and O75 antigen polysaccharides comprise the structures of Formulas (O1A), (O2), (O4-Glc+), (O6A), (O15), (O16), (O18A), (O25B), and (O75), respectively, as shown in Table 1, wherein each n is independently an integer of 1 to 100, preferably of 3 to 50, for example 5 to 40, preferably of 5 to 30, for example 7 to 25, for example 10 to 20, wherein the weight ratio of O75 antigen polysaccharide to O6 antigen polysaccharide is about 1.5:1 to about 4:1, more preferably about 2:1.

In certain embodiments according to a method of the invention, the E. coli O antigen polysaccharides administered to the subject consist of O1, O2, O4, O15, O16, O18, O25, O75 and O6.

In certain embodiments according to a method of the invention, the E. coli O antigen polysaccharides administered to the subject further comprise from 1 to 15 additional E. coli O antigen polysaccharides, each independently covalently linked to a carrier protein.

In certain embodiments according to a method of the invention, the subject is a human having or at risk of having an E. coli (preferably ExPEC) infection, preferably an invasive ExPEC disease.

In certain embodiments according to a method of the invention, 8-16 μg, preferably about 16 μg, of the O75 antigen polysaccharide is administered per administration.

In certain embodiments according to a method of the invention, the effective amount of the administered E. coli antigen polysaccharides of O1:O2:O4:O6:O15:O16:O18:O25:O75 is administered at a weight ratio of 1:1:1:1:1:1:1:2:2.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1A-FIG. 1B show antibody responses induced by ExPEC10V vaccine in New Zealand White rabbits. Animals received 3 intramuscular immunizations with ExPEC10V or saline administered 2 weeks apart. ExPEC10V vaccine was administered at 3 different concentrations (group 1: high dose, group 2: medium dose and group 3: low dose, Table 11) and a control group received only saline (group 4, 0.9% (w/v) sodium chloride solution). Antibody levels were measured by ELISA at day 0 (pre-vaccination) and days 14, 27 and 42 (post-vaccination). Individual titers (EC50 titer) and geometric mean titers (GMT)±95% CI are shown. Wilcoxon Rank Sum test with Bonferroni correction for multiple comparisons. Comparisons ExPEC10V vaccinated animals (group 1, 2 and 3) versus saline control (group 4). *p≤0.05, **p≤0.01; ***p≤0.001; ****p≤0.0001. LOD: limit of detection.

FIG. 2A-FIG. 2B show antibody responses induced by ExPEC10V. New Zealand White rabbits received 3 intramuscular immunizations with ExPEC10V (105.6 g total polysaccharide) or 0.9% w/v sodium chloride solution (control). IgG titers were determined by ELISA at day 1 (pre-immunization, n=20/group), day 31 (post-immunization, n=20/group) and day 50 (post-immunization, n=10/group). Plots show individual titers and geometric mean±95% confidence interval for each group. Differences in IgG titers between the ExPEC10V and control group were analyzed using a Tobit model with a likelihood ratio test. P-values≤0.05 were considered significant. *P<0.05, ****P<0.0001.

FIG. 3A-FIG. 3B show the overall study design for a phase 1/2a clinical trial with ExPEC10V vaccine in humans. FIG. 3A shows the overall study design for Cohort 1, and FIG. 3B shows the overall study design for Cohort 2. See Example 7 for details.

FIG. 4 shows the percentage of participants with at least 2-fold increase in serum O-antigen specific antibody titers at day 15 post-vaccination as measured from baseline (day 1) in the BAC1001 clinical trial (see Example 7 for details). O-antigen specific serum antibody titers were measured by ECL-based immunoassay.

FIG. 5 shows the percentage of participants with at least 2-fold increase in serum opsonophagocytic antibody titers at day 15 post-vaccination as measured from baseline (day 1) in the BAC1001 clinical trial (see Example 7 for details). Levels of serum opsonophagocytic antibodies were measured by MOPA.

FIG. 6A-FIG. 6B serum antibody levels (total IgG) induced by different compositions of ExPEC vaccine in New Zealand White rabbits. See Example 8 for details. Graphs show individual titers (EC50 titer) and geometric mean titers (GMT)±95% confidence intervals. Comparison between vaccine groups versus saline control were statistically evaluated using a Tobit model with likelihood ratio test and a Bonferroni correction for multiple comparisons was used. p values≤0.05 were considered statistically significant. ****p≤0.0001.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification. It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series.

The term “about,” when used in conjunction with a number, refers to any number within ±10%, e.g. ±5%, or ±1%, of the referenced number.

Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the terms “O polysaccharide,” “O-antigen”, “O-antigen”, “O-antigen polysaccharide,” “O-polysaccharide antigen” and the abbreviation “OPS”, all refer to the O-antigen of Gram-negative bacteria, which is a component of the lipopolysaccharide (LPS) and is specific for each serotype or sero(sub)type of the Gram-negative bacteria. The O-antigen usually contains repeating units (RUs) of two to seven sugar residues. As used herein, the RU is set equal to the biological repeat unit (BRU). The BRU describes the RU of an O-antigen as it is synthesized in vivo. Different serotypes of E. coli express different O-antigens. In E. coli, the gene products involved in O-antigen biogenesis are encoded by the rfb gene cluster. Whenever referring to an O-antigen polysaccharide herein, the O-antigen polysaccharide of the respective E. coli serotype and any existing subserotypes thereof are meant unless indicated otherwise, e.g. when referring to O1 antigen polysaccharide, this can be O-antigen polysaccharide of E. coli subserotypes O1A, O1A1, O1B, or O1C, while O25 antigen polysaccharide can mean O25A or O25B antigen polysaccharide, etc. Many E. coli serotypes and subserotypes as well as the corresponding structure of a RU (moiety structure, or O-unit) of the O-antigen polysaccharides thereof are provided in Table 1 of WO 2020/039359, incorporated by reference herein.

As used herein, “rfb cluster” and “rfb gene cluster” refer to a gene cluster that encodes enzymatic machinery capable of synthesizing an O-antigen backbone structure. The term rfb cluster can apply to any O-antigen biosynthetic cluster, and preferably refers to a gene cluster from the genus Escherichia, particularly E. coli.

As used herein, the term “O1A” refers to the O1A antigen of E. coli (a subserotype of E. coli serotype O1). The term “O2” refers to the O2 antigen of E. coli (E. coli serotype O2). The term “O4” refers to the O4 antigen of E. coli (E. coli serotype O4). The term “O6A” refers to the O6A antigen of E. coli (a subserotype of E. coli serotype O6). The term “O8” refers to the O8 antigen of E. coli (E. coli serotype O8). The term “O15” refers to the O15 antigen of E. coli (E. coli serotype O15). The term “O16” refers to the O16 antigen of E. coli (E. coli serotype O16). The term “O18A” refers to the O18A antigen of E. coli (a subserotype of E. coli serotype O18). The term “O25B” refers to the O25B antigen from E. coli (a subserotype of E. coli serotype O25). The term “O75” refers to the O75 antigen of E. coli (E. coli serotype O75). As used herein, the terms “glucosylated O4”, “glucose-branched O4”, “O4 Glc+” and “Glc+O4” 0-antigen refer to O4 O-antigen of E. coli (E. coli serotype O4) with a glucose side-branch, while “non-glucosylated O4,” “O4 Glc-,” and “Glc-O4” refer to an O4 antigen without a glucose side-branch.

The structures of several E. coli O-antigen polysaccharides referred to throughout this application are shown below in Table 1. A single repeating unit for each E. coli O-antigen polysaccharide is shown.

TABLE 1
Structures of E. coli O-antigen Polysaccharides

| E. coli O-antigen | |
|---|---|
| Polysaccharide | Structure of Repeating Unit¹ |
| O1A antigen polysaccharide (O1A) | 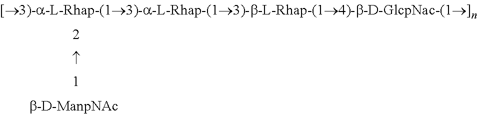 |
| O2 antigen polysaccharide (O2) | 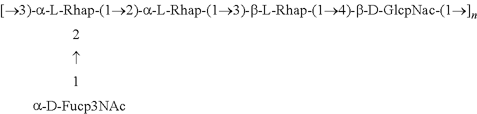 |
| Non-glucosylated O4 antigen | 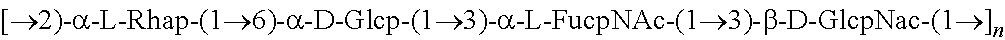 |
| polysaccharide (O4- | |
| Glc−) | |
| Glucosylated O4 antigen polysaccharide (O4- Glc+) | 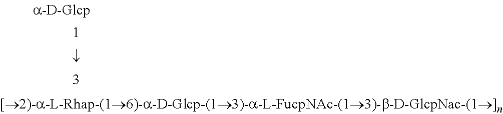 |
| O6A antigen polysaccharide (O6A) | 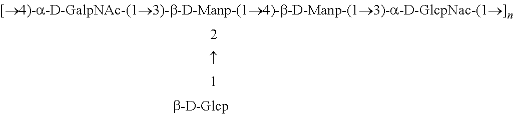 |
| O8 antigen polysaccharide (O8) | 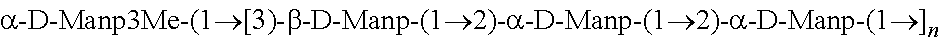 |
| O15 antigen polysaccharide | 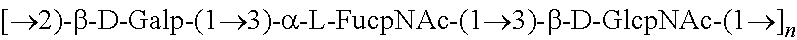 |
| (O15) | |
| O16 antigen polysaccharide (O16) | 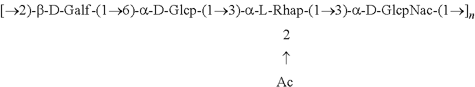 |
| O18A antigen polysaccharide (O18A) | 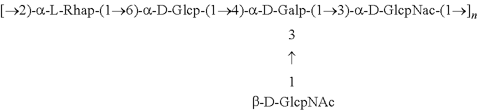 |
| O25B antigen polysaccharide (O25B) | 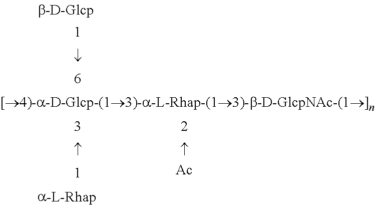 |
| O75 antigen polysaccharide (O75) | 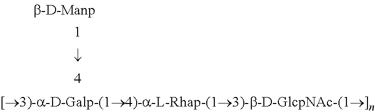 |

¹Each n is independently an integer of 1 to 100, such as 1-50, 1-40, 1-30, 1-20, and 1-10, 3-50, 3-40, 5-30, e.g. at least 5, such as 5-
40, e.g. 7-30, e.g. 7 to 25, e.g. 10 to 20, but in some instances can be 1-2.

All monosaccharides described herein have their common meaning known in the art. Monosaccharides can have the D or L configuration. If D or L is not specified, the sugar is understood to have the D configuration. Monosaccharides are typically referred to by abbreviations commonly known and used in the art. For example, Glc refers to glucose; D-Glc refers to D-glucose; and L-Glc refers to L-glucose. Other common abbreviations for monosaccharides include: Rha, rhamnose; GlcNAc, N-acetylglucosamine; GalNAc, N-acetylgalactosamine; Fuc, fucose; Man, mannose; Man3Me, 3-O-methyl-mannose; Gal, galactose; FucNAc, N-acetylfucosamine; and Rib, ribose. The suffix “f” refers to furanose and the suffix “p” refers to pyranose.

The terms “RU,” “repeat unit,” and “repeating unit” as used with respect to an O-antigen refer to the biological repeat unit (BRU) of an O-antigen as it is synthesized in vivo by cellular machinery (e.g., glycosyltransferases). The number of RUs of an O-antigen may vary per serotype, and in embodiments of the invention typically varies from about 1-100 RUs, preferably about 1 to 50 RUs, such as 1-50 RUs, 1-40 RUs, 1-30 RUs, 1-20 RUs, and 1-10 RUs, and more preferably at least 3 RUs, at least 4 RUs, at least 5 RUs, such as 3-50 RUs, preferably 5-40 RUs, preferably 5-30 RUs, e.g. 7-25 RUs, e.g. 10-20 RUs. However, in some instances, the number of RUs of an O-antigen can be 1-2. The structure of each O-antigen that is specifically described herein is shown containing one RU with the variable “n” designating the number of RUs. In each O-antigen polysaccharide in a bioconjugate of the invention, n is independently an integer of 1-100, such as 1-50, 1-40, 1-30, 1-20, 1-10, preferably at least 3, more preferably at least 5, such as 3-50, preferably 5-40 (e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40), more preferably 5-30, but in some instances can be 1-2. In some embodiments n is independently an integer of about 7-25, e.g. about 10-20. The values may vary between individual O-antigen polysaccharides in a composition, and are provided here as average values, i.e. if a bioconjugate is described herein as having an n that is independently an integer of 5-40, the composition contains a majority of O-antigen polysaccharides with 5-40 repeat units, but may also contain some O-antigen polysaccharides that have less than 5 repeat units or more than 40 repeat units.

As used herein, the terms “conjugate” and “glycoconjugate” refer to a sugar or saccharide antigen (e.g., oligo- and polysaccharide)-protein conjugate linked to another chemical species, including but not limited to proteins, peptides, lipids, etc. Glycoconjugates can be prepared chemically, e.g., by chemical (synthetic) linkage of the protein and sugar or saccharide antigen. The term glycoconjugate also includes bioconjugates.

As used herein, the term “effective amount” in the context of administering an O-antigen to a subject in methods according to embodiments of the invention refers to the amount of the O-antigen that is sufficient to induce a desired immune effect or immune response in the subject. In certain embodiments, an “effective amount” refers to the amount of an O-antigen which is sufficient to produce immunity in a subject to achieve one or more of the following effects in the subject: (i) prevent the development or onset of an ExPEC infection, preferably an invasive ExPEC disease, or symptom associated therewith; (ii) prevent the recurrence of an ExPEC infection, preferably an invasive ExPEC disease, or symptom associated therewith; (iii) prevent, reduce or ameliorate the severity of an ExPEC infection, preferably an invasive ExPEC disease, or symptom associated therewith; (iv) reduce the duration of an ExPEC infection, preferably an invasive ExPEC disease, or symptom associated therewith; (v) prevent the progression of an ExPEC infection, preferably an invasive ExPEC disease, or symptom associated therewith; (vi) cause regression of an ExPEC infection or symptom associated therewith; (vii) prevent or reduce organ failure associated with an ExPEC infection; (viii) reduce the chance or frequency of hospitalization of a subject having an ExPEC infection; (ix) reduce hospitalization length of a subject having an ExPEC infection; (x) increase the survival of a subject with an ExPEC infection, preferably an invasive ExPEC disease; (xi) eliminate an ExPEC infection, preferably an invasive ExPEC disease; (xii) inhibit or reduce ExPEC replication; and/or (xiii) enhance or improve the prophylactic or therapeutic effect(s) of another therapy.

An “effective amount” can vary depending upon a variety of factors, such as the physical condition of the subject, age, weight, health, etc.; route of administration, such as oral or parenteral; the composition administered, such as the target O-antigen, the other co-administered O-antigens, adjuvant, etc.; and the particular disease for which immunity is desired. When the O-antigen is covalently bound to a protein carrier, the effective amount for the O-antigen is calculated based on only the O-antigen polysaccharide moiety in the conjugate. All concentrations, amounts, and ratios of conjugates, including bioconjugates, as described herein, are also calculated based only on the weight of the O-antigen polysaccharide moieties in the conjugates, regardless of the concentration, amount, or ratio of any conjugated carrier proteins, unless indicated otherwise. For example, administration of 16 μg of a particular bioconjugate means that the administered bioconjugate comprises 16 μg of the particular O-antigen polysaccharide, and the amount of the conjugated carrier protein is not included in this number. For another example, if a composition is said to comprise conjugates of O-antigen polysaccharides from serotypes A and B in a ratio of 2:1, it indicates that there is two times the concentration or amount of the conjugated O-antigen polysaccharide A than that of the conjugated O-antigen polysaccharide B based on the weight of the conjugated O-antigen polysaccharides, disregarding the weight of the conjugated carrier proteins, in the composition.

The term “Invasive Extraintestinal pathogenic Escherichia coli (ExPEC) disease (IED)” as used herein is an acute illness consistent with systemic bacterial infection, which is microbiologically confirmed either by the isolation and identification of E. coli from blood or other normally sterile body sites, or by the isolation and identification of E. coli from urine in a patient with presence of signs and symptoms of invasive disease (systemic inflammatory response syndrome (SIRS), sepsis or septic shock) and no other identifiable source of infection. In certain embodiments, IED is an acute illness consistent with systemic bacterial infection, which is microbiologically confirmed either by (i) the isolation and identification of E. coli from blood or other normally sterile body sites, or by (ii) the isolation and identification of E. coli from urine in a patient with life threatening organ dysfunction due to dysregulated host response to infection originating from the urinary tract and/or male genital organs and no other identifiable source of infection.

IED may include, but is not necessarily limited to, urinary tract infection (UTI), a surgical-site infection, an abdominal or pelvic infection, pneumonia, osteomyelitis, cellulitis, sepsis, bacteremia, a wound infection, pyelonephritis, prostate biopsy-related infection (such as transrectal ultrasound-guided prostate needle biopsy [TRUS-PNB] related infection), urosepsis, meningitis, peritonitis, cholangitis, soft-tissue infections, pyomyositis, septic arthritis, endophthalmitis, suppurative thyroiditis, sinusitis, endocarditis, neutropenic fever, and prostatitis (including but not limited to acute bacterial prostatitis [ABP]).

In certain preferred embodiments, IED comprises sepsis. In certain preferred embodiments, IED comprises bacteremia. The invention in certain embodiments provides a composition according to the invention for preventing sepsis caused by E. coli. The invention in certain embodiments provides a composition according to the invention for preventing bacteremia caused by E. coli.

The term “IED event meeting criteria for sepsis” indicates an IED case including evidence of life-threatening organ dysfunction due to dysregulated host response to infection. A case of IED is meeting criteria for sepsis if there is an acute change in total Sequential Organ Failure Assessment (SOFA) score of 2 points or greater from baseline and deemed secondary to the IED. The invention in certain embodiments provides a composition according to the invention for preventing IED meeting the criteria for sepsis. The term “urosepsis” as used herein is sepsis caused by an infection originating from the urogenital tract and/or male genital organs.

The term “bacteremic IED” is an IED case which includes isolation and identification of E. coli from blood. The invention in certain embodiments provides a composition according to the invention for preventing bacteremic IED.

As used herein, an “immunological response” or “immune response” to an antigen or composition refers to the development in a subject of a humoral and/or a cellular immune response to the antigen or an antigen present in the composition.

As used herein, a “composition” comprising more than one E. coli antigen polysaccharide can be a single pharmaceutical composition that comprises the more than one E. coli antigen polysaccharide in the same pharmaceutical composition, or a combination of more than one pharmaceutical composition that comprises the more than one E. coli antigen polysaccharide in separate pharmaceutical compositions. In preferred embodiments, a composition is a single pharmaceutical composition. In a method of inducing an immune response to E. coli, a “composition” comprising more than one E. coli antigen polysaccharide can be administered to a subject in need thereof together in a single pharmaceutical composition that comprises the more than one E. coli antigen polysaccharide, or can be administered to the subject in combination in separate pharmaceutical compositions. In preferred embodiments, a single pharmaceutical composition is administered to the subject.

As used herein, the terms “in combination,” or “a combination of” in the context of the administration of two or more O-antigens or compositions to a subject, does not restrict the order in which O-antigens or compositions are administered to a subject. For example, a first composition can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second composition to a subject. Preferably the two or more O-antigens are administered to a subject essentially simultaneously, e.g. within five minutes of each other, and more preferably the two or more O-antigens are administered simultaneously via administration of at least two compositions at the same time, most preferably via administration of a single composition that comprises the two or more O-antigens.

As used herein, “subject” means any animal, preferably a mammal, most preferably a human, to who will be or has been vaccinated by a method or composition according to an embodiment of the invention. The term “mammal” as used herein, encompasses any mammal. Examples of mammals include, but are not limited to, cows, horses, sheep, pigs, cats, dogs, mice, rats, rabbits, guinea pigs, monkeys, humans, etc., most preferably a human. The terms “subject” and “patient” may be used herein interchangeably.

The term “percent (%) sequence identity” or “% identity” describes the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 95%, 97% or 98% identity) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

For example, for amino acid sequences, sequence identity and/or similarity can be determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al, 1984, Nucl. Acid Res. 12:387-395, preferably using the default settings, or by inspection. In certain embodiments, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

Another example of a useful algorithm is the BLAST algorithm, described in: Altschul et al, 1990, J. Mol. Biol. 215:403-410; Altschul et al, 1997, Nucleic Acids Res. 25:3389-3402; and Karin et al, 1993, Proc. Natl. Acad. Sci. U.S.A. 90:5873-5787. A particularly useful BLAST program is the WU-BLAST-2 program which was obtained from Altschul et al, 1996, Methods in Enzymology 266:460-480. WU-BLAST-2 uses several search parameters, most of which are set to the default values.

An additional useful algorithm is gapped BLAST as reported by Altschul et al, 1993, Nucl. Acids Res. 25:3389-3402.

E. coli O-Antigens

It has been surprisingly discovered in the invention that E. coli O75 antigen conjugated to a carrier protein appears to be less immunogenic than other E. coli O-antigens (e.g., O1A, O2, O4, O6A, O15, O16 and O18A) conjugated to the carrier protein at the same polysaccharide concentration in a multivalent vaccine composition. This discovery led to further investigation into the dosage of E. coli O75 antigen and the dosage ratios of various E. coli O-antigens within a multivalent vaccine, thus the development of multivalent vaccines and immunization methods based on E. coli O-antigens for improved immune responses against the O75 serotype and other serotypes of ExPEC.

Embodiments of the invention relate to compositions and methods relating to E. coli O75 antigen polysaccharide and one or more additional E. coli O-antigens polysaccharides. Preferably, the one or more additional O-antigens are prevalent among the clinical isolates of E. coli. Examples of such E. coli antigens that can be used in the invention include, but are not limited to, the E. coli O1, O2, O4, O6, O8, O15, O16, O18, and O25 antigens. Depending on the need, the composition can include more than one additional E. coli O antigens, such as two, three, four, five, six, seven, eight, nine, ten, eleven, twelve, thirteen, fourteen or fifteen additional E. coli O antigens, to provide immune protection against multiple E. coli serotypes in addition to the E. coli O75 serotype. In some embodiments, the additional E. coli O-antigen is selected from the group consisting of E. coli O1, O2, O4, O6, O15, O16, O18 and O25 antigens. In preferred embodiments, the additional E. coli O-antigen is selected from the group consisting of O1A, O2, glucosylated O4, O6A, O15, O16, O18A and O25B. More preferably, the composition includes all of O1A, O2, glucosylated O4, O6A, O15, O16, O18A and O25B E. coli O-antigens. In a particular embodiment, the E. coli O-antigens comprise the structures as shown in Table 1, wherein n is an integer of 1 to 100. In some embodiments, n is an integer of 3 to 50, e.g. 5 to 40, preferably of 5 to 30, e.g. 7 to 25, e.g. 10 to 20.

In one embodiment, a composition of the invention comprises E. coli O75 and O6 antigen polysaccharides, wherein each of the antigen polysaccharides is independently covalently linked to a carrier protein, and wherein the ratio of concentrations of O75 antigen polysaccharide to O6 antigen polysaccharide is about 1.2:1 to about 8:1, preferably about 1.5:1 to about 4:1, more preferably about 2:1, e.g. about 1:5:1, 1.6:1, 1.7:1, 1.8:1, 1.9:1, 2.0:1, 2.1:1, 2.2:1, 2.3:1, 2.4:1 or 2.5:1. In some embodiments, the composition further comprises one or more, preferably all, of E. coli O1, O2, O4, O15, O16, O18, O25 antigen polysaccharides, wherein each of the antigen polysaccharides is independently covalently linked to a carrier protein, preferably the O1 antigen is O1A, the O4 is glucosylated, the O6 antigen is O6A, the O18 antigen is O18A, and the O25 antigen is O25B.

An E. coli O-antigen useful in the invention can be produced by methods known in the art in view of the present disclosure. For example, they can be produced from a cell, preferably a recombinant cell that is optimized for the biosynthesis of the O-antigen. See, e.g., relevant disclosure on the nucleic acids, proteins, host cells, production methods, etc., for E. coli O-antigen biosynthesis in WO 2006/119987, WO 2009/104074, International Patent Application No. PCT/EP2015/053739, Ihssen et al., 2010, Microbial Cell Factories 9, 61, the disclosures of which are herein incorporated by reference in their entirety.

### Carrier Proteins

According to embodiments of the invention, each E. coli O-antigen is covalently bound to a carrier protein, preferably by a glycosidic linkage. Any carrier protein known to those skilled in the art in view of the present disclosure can be used. Suitable carrier proteins include, but are not limited to, detoxified Exotoxin A of P. aeruginosa (EPA), CRM₁₉₇, E. coli flagellin (FliC), maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae. Bioconjugation with various different carrier proteins containing the required consensus glycosylation sequence has been described, showing that a wide range of proteins can be glycosylated using this technology (see, e.g. WO O6/119987, WO 2015/124769, WO 2015/158403, WO 2015/82571, WO 2017/216286, and WO 2017/67964, together showing a wide variety of carrier proteins that were successfully used in bioconjugation).

In certain embodiments a carrier protein is modified, e.g., modified in such a way that the protein is less toxic and/or more susceptible to glycosylation. In a specific embodiment, the carrier proteins used herein are modified such that the number of glycosylation sites in the carrier proteins is maximized in a manner that allows for lower concentrations of the protein to be administered, e.g., in an immunogenic composition, particularly in its bioconjugate form.

Thus, in certain embodiments, the carrier proteins described herein are modified to include 1, 2, 3, 4, 5, 6, 7, 8, 9, 10 or more glycosylation sites than would normally be associated with the carrier protein (e.g., relative to the number of glycosylation sites associated with the carrier protein in its native/natural, i.e., “wild-type” state). Introduction of glycosylation sites into a carrier protein can be accomplished by insertion of a glycosylation consensus sequence anywhere in the primary structure of the protein by, e.g., adding new amino acids to the primary structure of the protein such that a glycosylation site is added in full or in part, or by mutating existing amino acids in the protein in order to generate a glycosylation site. One of ordinary skill in the art will recognize that the amino acid sequence of a protein can be readily modified using approaches known in the art, e.g., recombinant approaches that include modification of the nucleic acid sequence encoding the protein. In specific embodiments, glycosylation consensus sequences are introduced into specific regions of the carrier protein, e.g., surface structures of the protein, at the N or C termini of the protein, and/or in loops that are stabilized by disulfide bridges at the base of the protein. In some embodiments, a glycosylation consensus sequence can be extended by addition of lysine residues for more efficient glycosylation.

Exemplary examples of glycosylation consensus sequences that can be inserted into or generated in a carrier protein include Asn-X-Ser(Thr), wherein X can be any amino acid except Pro (SEQ ID NO: 1); and preferably Asp(Glu)-X-Asn-Z-Ser(Thr), wherein X and Z are independently selected from any amino acid except Pro (SEQ ID NO: 2).

In some embodiments, the E. coli O-antigen polysaccharide is covalently linked to an asparagine (Asn) residue in the carrier protein (e.g., N-linked), wherein the Asn residue is present in a glycosylation site comprising a glycosylation consensus sequence having SEQ ID NO: 1, more preferably having SEQ ID NO: 2. Typically, a carrier protein comprises 1-10 glycosylation sites, preferably 2 to 4 glycosylation sites, most preferably 4 glycosylation sites, such as 1-10, preferably 2-4, and more preferably 4 glycosylation sites each comprising a glycosylation consensus sequences having the amino acid sequence of SEQ ID NO: 1, and more preferably the amino acid sequence of SEQ ID NO: 2.

In particular embodiments, a carrier protein is a detoxified Exotoxin A of P. aeruginosa. For EPA, various detoxified protein variants have been described in literature and could be used as carrier proteins. For example, detoxification can be achieved by mutating and deleting the catalytically essential residues L552V and ΔE553 according to Lukac et al., 1988, Infect Immun, 56: 3095-3098, and Ho et al., 2006, Hum Vaccin, 2:89-98. As used herein, “EPA” refers to a detoxified Exotoxin A of P. aeruginosa. In those embodiments wherein the carrier protein is EPA, an E. coli antigen polysaccharide can be covalently linked to an Asn residue in a glycosylation site comprising a glycosylation consensus sequence having SEQ ID NO: 1, and preferably covalently linked to an Asn residue in a glycosylation site comprising a glycosylation consensus sequence having SEQ ID NO: 2. Preferably, the EPA carrier protein comprises 1-10 glycosylation sites, preferably 2 to 4 glycosylation sites, most preferably 4 glycosylation sites, such as 1-10, preferably 2-4, and more preferably 4 glycosylation sites each comprising a glycosylation consensus sequence having the amino acid sequence of SEQ ID NO: 1, and more preferably the amino acid sequence of SEQ ID NO: 2.

In some embodiments, the EPA carrier protein comprises four glycosylation sites each comprising a glycosylation consensus sequence, for instance a glycosylation site comprising a glycosylation consensus sequence having SEQ ID NO: 2. As used herein, “EPA-4 carrier protein” and “EPA-4” refer to a detoxified Exotoxin A of P. aeruginosa carrier protein comprising four glycosylation sites each comprising a glycosylation consensus sequences having SEQ ID NO: 2. An exemplary preferred example of an EPA-4 carrier protein is EPA carrier protein comprising the amino acid sequence of SEQ ID NO: 3.

In certain embodiments, the EPA carrier protein can be produced together with a signal sequence (such as a signal peptide for E. coli DsbA, E. coli outer membrane porin A (OmpA), E. coli maltose binding protein (MalE), etc.) that targets the carrier protein to the periplasmic space of the host cell that expresses the carrier protein. The EPA carrier protein can also be modified to a “tag,” i.e., a sequence of amino acids that allows for the isolation and/or identification of the carrier protein.

An EPA carrier protein useful in the invention can be produced by methods known in the art in view of the present disclosure. See, e.g., relevant disclosure in e.g., Ihssen et al., 2010, Microbial Cell Factories 9, 61, and in WO 2006/119987, WO 2009/104074, and WO 2015/124769, the disclosures of which are herein incorporated by reference in their entireties.

In other embodiments, a carrier protein is CRM₁₉₇. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{lox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. The amino acid sequence of a CRM₁₉₇ protein is shown in SEQ ID NO. 20. Further details about CRM₁₉₇ and production thereof can be found, e.g., in U.S. Pat. No. 5,614,382. In an embodiment, an O-antigen polysaccharide of the invention is conjugated to CRM₁₉₇ protein or the A chain of CRM₁₉₇ (see CN103495161). In an embodiment, an O-antigen polysaccharide of the invention is conjugated the A chain of CRM₁₉₇ obtained via expression by genetically recombinant E. coli (see CN103495161). In an embodiment, the O-antigen polysaccharides of the invention are each independently conjugated to CRM₁₉₇. In an embodiment, the O-antigen polysaccharides of the invention are each independently conjugated to the A chain of CRM₁₉₇.

### Conjugates

The term “bioconjugate” refers to a conjugate between a protein (e.g., a carrier protein) and a sugar or saccharide antigen (e.g., oligo- and polysaccharide) prepared in a host cell background, preferably a bacterial host cell, e.g. an E. coli host cell, wherein host cell machinery links the antigen to the protein (e.g., N-links). Preferably, the term “bioconjugate” refers to a conjugate between a protein (e.g., carrier protein) and an O-antigen, preferably an E. coli O-antigen (e.g., O1A, O2, O4, O6A, O8, O15, O16, O18A, O25B, O75, etc.) prepared in a host cell background, wherein host cell machinery links the antigen to the protein (e.g., N-links). Because bioconjugates are prepared in host cells by host cell machinery, the antigen and protein are covalently linked via a glycosidic linkage or bond in a bioconjugate. Bioconjugates can be prepared in recombinant host cells engineered to express the cellular machinery needed to synthesize the O-antigen and/or link the O-antigen to the target protein. Bioconjugates, as described herein, have advantageous properties over chemically prepared glycoconjugates where the glycans are purified from bacterial cell walls and subsequently chemically coupled to a carrier protein, e.g., bioconjugates require fewer chemicals in manufacture and are more consistent in terms of the final product generated, and contain less or no free (i.e. unbound to carrier protein) glycan. Purification of O-antigen free from lipid A and subsequent chemical conjugation to a carrier protein is a lengthy and laborious process. Additionally, the purification, lipid A detoxification and chemical conjugation processes can result in loss of epitopes, antigen heterogeneity and reduced immunogenicity of the conjugated polysaccharide. Synthesis of glycoconjugates by bioconjugation can overcome these limitations of classical purification and chemical conjugation. Thus, in typical embodiments, bioconjugates are preferred over chemically produced glycoconjugates.

In certain embodiments, a host cell can produce an E. coli O-antigen and an EPA carrier protein, and covalently bind the O-antigen to the EPA carrier protein to form a bioconjugate useful in the invention. See, e.g., relevant disclosure in e.g., Ihssen et al., 2010, Microbial Cell Factories 9, 61, and in WO 2006/119987, WO 2009/104074, and WO 2015/124769, the disclosures of which are herein incorporated by reference in their entirety.

In a specific embodiment, the carrier protein is N-linked to an E. coli O-antigen useful in the invention. For example, the E. coli O-antigen is linked to the Asn residue in a glycosylation sequence of a carrier protein, such as Asn-X-Ser(Thr), wherein X can be any amino acid except Pro (SEQ ID NO: 2), preferably Asp(Glu)-X-Asn-Z-Ser(Thr), wherein X and Z are independently selected from any natural amino acid except Pro (SEQ ID NO: 3).

Alternatively, the glycoconjugates can be prepared by chemical synthesis, i.e., prepared outside of host cells (in vitro). For example, the E. coli O-antigens described herein, e.g., O75 antigen, can be conjugated to carrier proteins using methods known to those of skill in the art, including by means of using activation reactive groups in the polysaccharide/oligosaccharide as well as the protein carrier. See, e.g., Pawlowski et al., 2000, Vaccine 18:1873-1885; and Robbins et al., 2009, Proc Natl Acad Sci USA 106:7974-7978, the disclosures of which are herein incorporated by reference. Such approaches comprise extraction of antigenic polysaccharides/oligosaccharides from host cells, purifying the polysaccharides/oligosaccharides, chemically activating the polysaccharides/oligosaccharides, and conjugating the polysaccharides/oligosaccharides to a carrier protein. Methods to make glycoconjugates of E. coli O-antigens conjugated to carrier proteins using chemical conjugation to carrier protein, and compositions comprising such glycoconjugates, have also been described in WO 2020/039359.

For example, conjugates can be prepared using CDAP chemistry. In these embodiments, the polysaccharides are activated with 1-cyano-4-dimethylamino pyridinium tetrafluoroborate (CDAP) to form a cyanate ester. The activated polysaccharide is then coupled directly or via a spacer (linker) group to an amino group on the carrier protein (e.g., EPA or CRM₁₉₇). For example, the spacer could be cystamine or cysteamine to give a thiolated polysaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., EPA or CRM₁₉₇) using carbodiimide (e.g., EDAC or EDC) chemistry via a carboxyl group on the protein carrier. Such conjugates are described for example in WO 93/15760, WO 95/08348 and WO 96/129094.

Other suitable techniques for conjugation use carbodiimides, hydrazides, active esters, norborane, p-nitrobenzoic acid, N-hydroxysuccinimide, S—NHS, EDC, TSTU. Many are described in WO 98/42721. Conjugation may involve a carbonyl linker which may be formed by reaction of a free hydroxyl group of the saccharide with CDI (see Bethell et al. (1979) 1. Biol. Chem. 254:2572-2574; Hearn et al. (1981) J. Chromatogr. 218:509-518) followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein.

In other embodiments, conjugates are prepared using reductive amination. Reductive amination involves two steps, (1) oxidation of the polysaccharide, (2) reduction of the activated polysaccharide and a carrier protein to form a conjugate. Before oxidation, the polysaccharide is optionally hydrolyzed. Mechanical or chemical hydrolysis maybe employed. Chemical hydrolysis can be conducted using acetic acid.

The oxidation step can involve reaction with periodate. For the purpose of the present invention, the term “periodate” includes both periodate and periodic acid; the term also includes both metaperiodate (IO₄⁻) and orthoperiodate (IO₆⁵⁻) and includes the various salts of periodate (e.g., sodium periodate and potassium periodate). In an embodiment, the capsular polysaccharide is oxidized in the presence of metaperiodate, preferably in the presence of sodium periodate (NaIO₄). In another embodiment the capsular polysaccharide is oxidized in the presence of orthoperiodate, preferably in the presence of periodic acid.

In an embodiment, the oxidizing agent is a stable nitroxyl or nitroxide radical compound, such as piperidine-N-oxy or pyrrolidine-N-oxy compounds, in the presence of an oxidant to selectively oxidize primary hydroxyls (as described in WO 2014/097099). In said reaction, the actual oxidant is the N-oxoammonium salt, in a catalytic cycle. In an aspect, said stable nitroxyl or nitroxide radical compound are piperidine-N-oxy or pyrrolidine-N-oxy compounds. In an aspect, said stable nitroxyl or nitroxide radical compound bears a TEMPO (2,2,6,6-tetramethyl-1-piperidinyloxy) or a PROXYL (2,2,5,5-tetramethyl-1-pyrrolidinyloxy) moiety. In an aspect, said stable nitroxyl radical compound is TEMPO or a derivative thereof. In an aspect, said oxidant is a molecule bearing a N-halo moiety. In an aspect, said oxidant is selected from the group consisting of N-ChloroSuccinimide, N-Bromosuccinimide, N-Iodosuccinimide, Dichloroisocyanuric acid, 1,3,5-trichloro-1,3,5-triazinane-2,4,6-trione, Dibromoisocyanuric acid, 1,3,5-tribromo-1,3,5-triazinane-2,4,6-trione, Diiodoisocyanuric acid and 1,3,5-triiodo-1,3,5-triazinane-2,4,6-trione. Preferably said oxidant is N-Chlorosuccinimide.

Optionally the oxidation reaction is quenched by addition of a quenching agent. The quenching agent is selected from vicinal diols, 1,2-aminoalcohols, amino acids, glutathione, sulfite, bisulfate, dithionite, metabisulfite, thiosulfate, phosphites, hypophosphites or phosphorous acid (such as glycerol, ethylene glycol, propan-1,2-diol, butan-1,2-diol or butan-2,3-diol, ascorbic acid).

Following the oxidation step of the polysaccharide, the polysaccharide is said to be activated and is referred to an “activated polysaccharide” here below. The activated polysaccharide and the carrier protein may be lyophilised (freeze-dried), either independently (discrete lyophilization) or together (co-lyophilized). In one embodiment the activated polysaccharide and the carrier protein are co-lyophilized. In another embodiment the activated polysaccharide and the carrier protein are lyophilized independently.

In one embodiment the lyophilization takes place in the presence of a non-reducing sugar, possible non-reducing sugars include sucrose, trehalose, raffinose, stachyose, melezitose, dextran, mannitol, lactitol and palatinit.

The second step of the conjugation process is the reduction of the activated polysaccharide and a carrier protein to form a conjugate (so-called reductive amination), using a reducing agent. Reducing agents which are suitable include the cyanoborohydrides (such as sodium cyanoborohydride, sodium triacetoxyborohydride or sodium or zinc borohydride in the presence of Bronsted or Lewis acids), amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB) or borohydride exchange resin. In one embodiment the reducing agent is sodium cyanoborohydride.

In an embodiment, the reduction reaction is carried out in aqueous solvent (e.g., selected from PBS, MES, HEPES, Bis-tris, ADA, PIPES, MOPSO, BES, MOPS, DIPSO, MOBS, HEPPSO, POPSO, TEA, EPPS, Bicine or HEPB, at a pH between 6.0 and 8.5, 7.0 and 8.0, or 7.0 and 7.5), in another embodiment the reaction is carried out in aprotic solvent. In an embodiment, the reduction reaction is carried out in DMSO (dimethylsulfoxide) or in DMF (dimethylformamide) solvent. The DMSO or DMF solvent may be used to reconstitute the activated polysaccharide and carrier protein which has been lyophilized.

At the end of the reduction reaction, there may be unreacted aldehyde groups remaining in the conjugates, these may be capped using a suitable capping agent. In one embodiment this capping agent is sodium borohydride (NaBH₄).

In other embodiments, conjugates are prepared using eTEC conjugation, such as described in WO 2014/027302. Said glycoconjugates comprise a saccharide covalently conjugated to a carrier protein through one or more eTEC spacers, wherein the saccharide is covalently conjugated to the eTEC spacer through a carbamate linkage, and wherein the carrier protein is covalently conjugated to the eTEC spacer through an amide linkage. The eTEC linked glycoconjugates of the invention may be represented by the general formula (I):

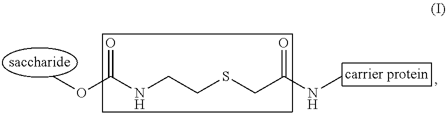

where the atoms that comprise the eTEC spacer are contained in the central box.

The eTEC spacer includes seven linear atoms (i.e., C(O)NH(CH₂)₂SCH₂C(O)—) and provides stable thioether and amide bonds between the saccharide and carrier protein. Synthesis of the eTEC linked glycoconjugate involves reaction of an activated hydroxyl group of the saccharide with the amino group of a thioalkylamine reagent, e.g., cystamine or cysteinamine or a salt thereof, forming a carbamate linkage to the saccharide to provide a thiolated saccharide. Generation of one or more free sulfhydryl groups is accomplished by reaction with a reducing agent to provide an activated thiolated saccharide. Reaction of the free sulfhydryl groups of the activated thiolated saccharide with an activated carrier protein having one or more α-haloacetamide groups on amine containing residues generates a thioether bond to form the conjugate, wherein the carrier protein is attached to the eTEC spacer through an amide bond.

The conjugates described herein can be purified by any method known in the art for purification of a protein, for example, by chromatography (e.g., ion exchange, anionic exchange, affinity, and sizing column chromatography), centrifugation, differential solubility, or by any other standard technique for the purification of proteins. See, e.g., Saraswat et al., 2013, Biomed. Res. Int. ID #312709 (p. 1-18); see also the methods described in WO 2009/104074. The actual conditions used to purify a particular conjugate will depend, in part, on the synthesis strategy (e.g., synthetic production vs. recombinant production) and on factors such as net charge, hydrophobicity, and/or hydrophilicity of the bioconjugate, and will be apparent to those having skill in the art.

### Host Cells

Described herein are host cells, e.g., prokaryotic host cells, capable of producing E. coli O antigens and bioconjugates comprising such E. coli O antigens. The host cells described herein preferably are modified to comprise (e.g., through genetic engineering) one or more of the nucleic acids encoding host cell machinery (e.g., glycosyltransferases) used to produce E. coli O-antigen polysaccharides and/or bioconjugates thereof.

Any host cells known to those of skill in the art can be used to produce the E. coli 0 antigen polysaccharides described herein (e.g., E. coli O75 polysaccharide) and bioconjugates comprising the E. coli O antigen polysaccharides described herein (e.g., a bioconjugate of E. coli O75 antigen polysaccharide) including archaea, prokaryotic host cells, and eukaryotic host cells. In a preferred embodiment, a host cell is a prokaryotic host cell. Exemplary prokaryotic host cells for use in production of the E. coli O antigen polysaccharides described herein and bioconjugates comprising the E. coli O antigen polysaccharides described herein include, but are not limited to, Escherichia species, Shigella species, Klebsiella species, Xhantomonas species, Salmonella species, Yersinia species, Lactococcus species, Lactobacillus species, Pseudomonas species, Corynebacterium species, Streptomyces species, Streptococcus species, Staphylococcus species, Bacillus species, and Clostridium species.

In a specific embodiment, the host cell used to produce the E. coli O antigen polysaccharides described herein and bioconjugates comprising the E. coli O antigen polysaccharides described herein is a prokaryotic host cell, and is preferably E. coli.

In certain embodiments, the host cells used to produce the E. coli O antigen polysaccharides and bioconjugates described herein are engineered to comprise heterologous nucleic acids, e.g., heterologous nucleic acids comprising rfb gene clusters of a desired O antigen serotype, heterologous nucleic acids that encode one or more carrier proteins and/or glycosyltransferases. In a specific embodiment, heterologous rfb genes, and/or heterologous nucleic acids that encode proteins involved in glycosylation pathways (e.g., prokaryotic and/or eukaryotic glycosylation pathways) can be introduced into the host cells described herein. Such nucleic acids can encode proteins including, but not limited to, oligosaccharyl transferases and/or glycosyltransferases.

Sequences of various genes and gene clusters encoding glycosyltransferases useful in making recombinant host cells that can, e.g., be used to prepare E. coli O antigen polysaccharides and bioconjugates thereof are described herein. Those skilled in the art will appreciate that due to the degeneracy of the genetic code, a protein having a specific amino acid sequence can be encoded by multiple different nucleic acids. Thus, those skilled in the art will understand that a nucleic acid provided herein can be altered in such a way that its sequence differs from a sequence provided herein, without affecting the amino acid sequence of the protein encoded by the nucleic acid.

Described herein are host cells (e.g., recombinant host cells) for producing a bioconjugate of an E. coli O75 antigen polysaccharide, O1A antigen polysaccharide, O2 antigen polysaccharide, glucosylated or non-glucosylated O4 antigen polysaccharide, O6A antigen polysaccharide, O8 antigen polysaccharide, O15 antigen polysaccharide, O16 antigen polysaccharide, O18A antigen polysaccharide, or O25B antigen polysaccharide. The host cells described herein comprise nucleic acids encoding enzymes (e.g., glycosyltransferases) capable of producing the E. coli O antigen polysaccharide. The host cells described herein can naturally express nucleic acids capable of producing an O antigen of interest, or the host cells can be made to express such nucleic acids. In certain embodiments the nucleic acids are heterologous to the host cells and introduced into the host cells using genetic approaches known in the art. For example, the nucleic acids can be introduced into the host cell by genetic manipulation (e.g., the gene cluster is expressed on a plasmid or plasmids or integrated into the host cell genome (see, e.g., WO 2014/037585, WO 2014/057109, WO 2015/052344).

Also described herein are host cells (e.g., recombinant host cells) capable of producing a bioconjugate of an E. coli O1A, O2, glucosylated or non-glucosylated O4, O6A, O8, O15, O16, O18A, O25B, or O75 antigen polysaccharide covalently linked to a carrier protein. Such host cells (e.g., recombinant host cells) comprise nucleotide sequence of an rfb gene cluster specific to the O-antigen polysaccharide. The rfb gene clusters can be isolated from wild-type E. coli strains, and combined with nucleic acids encoding an oligosaccharyl transferase (e.g., PglB) and carrier protein (e.g., EPA) within one host cell to obtain a recombinant host cell that produces the E. coli O-antigen of interest or bioconjugate thereof. For example, such host cells can be engineered using recombinant approaches to comprise one or more plasmids comprising the rfb gene cluster, oligosaccharyl transferase (e.g., PglB) and carrier protein (e.g., EPA) using bioconjugation technology such as that described in WO 2015/124769, WO 2014/037585, WO 2009/104074, and WO 2009/089396. Preferably the host cells comprise the rfb gene clusters integrated into their genome. The nucleic acids encoding oligosaccharyl transferase, carrier protein, and where applicable gtrS gene, are in certain embodiments also integrated into the genome of the host cell. Heterologous or homologous gtrA and gtrB genes are in certain embodiments also integrated into the genome of the host cell.

Preparation of bioconjugates for O1A, O2, O6A and O25B antigens has been described in detail in WO 2015/124769 and WO 2017/035181. Exemplary gene clusters for each E. coli O antigen (rfb loci) have been described in Iguchi A, et al, DNA Research, 2014, 1-7, and in DebRoy C, et al, PLoS One. 2016, 11(1):e0147434; correction in: Plos One. 2016, 11(4):e0154551). Nucleic acid sequences for the rfb clusters and amino acid sequences for proteins encoded therein can also be found in public databases, such as GenBank. Exemplary sequences for rfb clusters that can be used in production strains for bioconjugates with polysaccharide antigens of the serotypes disclosed herein, are also provided in SEQ ID NOs: 9 and 11-19. Thus, for each of the desired bioconjugates mentioned above, the respective rfb cluster can be introduced into a host cell, to obtain host cells with the specific rfb cluster for the desired O-antigen, as well as containing nucleic acid encoding oligosaccharyl transferase and carrier protein. For reasons indicated above, preferably the host cells are recombinant host cells, and preferably are derived from strains with relatively well-known characteristics, such as E. coli laboratory or production strains, e.g. E. coli K12 or E. coli BL21, etc. Preferably, the rfb clusters are heterologous to the host cell, e.g. introduced into a precursor cell of the host cell, and preferably integrated into the genome thereof. Preferably an original rfb gene cluster, if such was present in a precursor cell, has been replaced by the rfb gene cluster for the O-antigen of interest in the host cell, to enable production of bioconjugate of the O-antigen of interest. Preferably the oligosaccharyl transferase is heterologous to the host cell, and in certain embodiments nucleic acid encoding such oligosaccharyl transferase is integrated into the genome of the host cell.

Any of the host cells described herein (e.g., recombinant host cells, preferably recombinant prokaryotic host cells) comprise nucleic acids encoding additional enzymes active in the N-glycosylation of proteins, e.g., the host cell described herein can further comprise a nucleic acid encoding an oligosaccharyl transferase or nucleic acids encoding other glycosyltransferases.

The host cells described herein typically comprise a nucleic acid that encodes an oligosaccharyl transferase. Such oligosaccharyl transferases transfer lipid-linked oligosaccharides to asparagine residues of nascent polypeptide chains that comprise an N-glycosylation consensus motif. The nucleic acid that encodes an oligosaccharyl transferase can be native to the host cell, or can be introduced into the host cell using genetic approaches. In preferred embodiments, the oligosaccharyl transferase is heterologous to the host cell. E. coli does not naturally comprise an oligosaccharyl transferase, and hence if E. coli is used as a host cell for production of bioconjugates, a heterologous oligosaccharyl transferase is comprised in such host cell, e.g. upon introduction by genetic engineering. The oligosaccharyl transferase can be from any source known in the art in view of the present disclosure.

In certain embodiments, an alternative to an oligosaccharyl transferase with N-glycosyltransferase activity, such as an O-glycosyltransferase, e.g. as a non-limiting example PglL, can be used, in conjunction with its own, different, glycosylation consensus sequence in the carrier protein, as for instance described in WO 2016/82597 and WO 2020/120569. Other glycosyltransferases, such as O-glycosyltransferases, can thus also be used as an oligosaccharyl transferase according to the invention.

In certain preferred embodiments, the oligosaccharyl transferase is an oligosaccharyl transferase from Campylobacter. For example, in one embodiment, the oligosaccharyl transferase is an oligosaccharyl transferase from Campylobacter jejuni (i.e., pglB; see, e.g., Wacker et al., 2002, Science 298:1790-1793; see also, e.g., NCBI Gene ID: 3231775, UniProt Accession No. O86154). In another embodiment, the oligosaccharyl transferase is an oligosaccharyl transferase from Campylobacter lari (see, e.g., NCBI Gene ID: 7410986).

In specific embodiments, the oligosaccharyl transferase is PglB oligosaccharyl transferase from Campylobacter jejuni, including the natural (wild-type) protein or any variant thereof, such as those described in WO 2016/107818 and WO 2016/107819. PglB can transfer lipid-linked oligosaccharides to asparagine residues in the consensus sequences SEQ ID NO: 1 and SEQ ID NO: 2. In particular embodiments, the PglB oligosaccharyl transferase comprises SEQ ID NO: 6, or a variant thereof. In certain embodiments one or more endogenous glycosylation consensus sequences in a wild-type PglB have been mutated to avoid PglB autoglycosylation, e.g. SEQ ID NO: 6 comprising the mutation N534Q. Examples of variant PglB oligosaccharyl transferases suitable for use in the recombinant host cells provided herein include the PglB oligosaccharyl transferase of SEQ ID NO: 6 comprising at least one mutation selected from the group consisting of N311V, K482R, D483H, A669V, Y77H, S80R, Q287P, and K289R. In one particular embodiment, a variant PglB oligosaccharyl transferase has SEQ ID NO: 6 comprising the mutation N311V. In another particular embodiment, a variant PglB oligosaccharyl transferase has SEQ ID NO: 6 comprising the mutations Y77H and N311V. In another particular embodiment, a variant PglB oligosaccharyl transferase has SEQ ID NO: 6 comprising the mutations N311V, K482R, D483H, and A669V. In another particular embodiment, a variant PglB oligosaccharyl transferase has SEQ ID NO: 6 comprising the mutations Y77H, S80R, Q287P, K289R, and N311V. It was found and described in PCT/US20/23415, filed on 18 Mar. 2020, that certain PglB oligosaccharyl transferase variants give surprisingly improved yields in production of E. coli O-antigen bioconjugates of specific serotypes. The improved or optimal PglB variant for a given E. coli O-antigen was not predictable. The invention in certain aspects therefore also discloses methods for producing bioconjugates of specific E. coli O-antigens, using specific PglB variants as the oligosaccharyl transferase. Further variants of PglB that are at least 80%, 85%, 90%, 91%, 92%, 93%, 94%, 95%, 96%, 97%, 98%, 99% identical to SEQ ID NO: 6 and still have oligosaccharyl transferase activity, preferably having one or more of the specific amino acids on the indicated positions disclosed in combination herein (e.g. 77Y, 80S, 287Q, 289K, 311N, 482K, 483D, 669A; or 311V; or 311V, 482R, 483H, 669V; or 77H, 80R, 287P, 289R, 311V; or 77H, 311V; etc) can also be used for production of bioconjugates.

In a specific embodiment, a host cell (e.g., recombinant host cell) capable of producing a bioconjugate of an E. coli O75 antigen polysaccharide covalently linked to a carrier protein further comprises a nucleotide sequence encoding PglB oligosaccharyl transferase from Campylobacter jejuni having the amino acid sequence of SEQ ID NO: 6, or preferably SEQ ID NO: 6 comprising the mutation N311V.

In other specific embodiments, a host cell (e.g., recombinant host cell) capable of producing a bioconjugate of an E. coli O1A, O6A, or O15 antigen polysaccharide covalently linked to a carrier protein further comprises a nucleotide sequence encoding PglB oligosaccharyl transferase from Campylobacter jejuni having the amino acid sequence of SEQ ID NO: 6, or preferably SEQ ID NO: 6 comprising the mutations N311V, K482R, D483H, and A669V.

In a specific embodiment, a host cell (e.g., recombinant host cell) capable of producing a bioconjugate of an E. coli glucosylated O4 antigen polysaccharide covalently linked to a carrier protein further comprises a nucleotide sequence encoding PglB oligosaccharyl transferase from Campylobacter jejuni having the amino acid sequence of SEQ ID NO: 6, or preferably SEQ ID NO: 6 comprising the mutation N311V, or more preferably SEQ ID NO: 6 comprising the mutations Y77H and N311V. Preferably, the recombinant host cell for production of a bioconjugate of an E. coli glucosylated O4 antigen polysaccharide covalently linked to a carrier protein further comprises a sequence encoding a glucosyltransferase GtrS having the amino acid sequence of SEQ ID NO: 4 (or a variant thereof that is at least 80%, 85%, 90%, 91%, 92%, 93%, 94%, 95%, 96%, 97%, 98%, 99% identical to SEQ ID NO: 4) and being capable of modifying an E. coli O4 antigen polysaccharide by addition of glucose to produce the E. coli glucosylated O4 antigen polysaccharide, and nucleotide sequences encoding a translocase GtrA and a glycosyltransferase GtrB having the amino acid sequences of SEQ ID NOs: 7 and 8 respectively (or variants thereof with amino acid sequences that are at least 80%, 85%, 90%, 91%, 92%, 93%, 94%, 95%, 96%, 97%, 98%, 99% identical to SEQ ID NOs: 7 and 8 respectively), wherein the translocase is capable of translocating bactoprenol linked glucose and the glycosyltransferase is capable of glucosylating bactoprenol. For production of a bioconjugate of an E. coli non-glucosylated O4 antigen polysaccharide covalently linked to a carrier protein the cell does not require such sequences encoding GtrS, GtrA, and GtrB proteins. Production of bioconjugates of an E. coli glucosylated O4 antigen polysaccharide covalently linked to a carrier protein, using a novel GtrS specific for glucosylation of E. coli O4 antigen polysaccharide, is described in PCT/US20/23404, filed on 18 Mar. 2020, incorporated in its entirety by reference herein.

In a specific embodiment, a host cell (e.g., recombinant host cell) capable of producing a bioconjugate of an E. coli O16 antigen polysaccharide covalently linked to a carrier protein further comprises a nucleotide sequence encoding PglB oligosaccharyl transferase from Campylobacter jejuni having the amino acid sequence of SEQ ID NO: 6, or preferably SEQ ID NO: 6 comprising the mutations Y77H, S80R, Q287P, K289R, and N311V.

In a specific embodiment, a host cell (e.g., recombinant host cell) capable of producing a bioconjugate of an E. coli O8, O18A, O25B, or O2 antigen polysaccharide covalently linked to a carrier protein further comprises a nucleotide sequence encoding PglB oligosaccharyl transferase from Campylobacter jejuni having the amino acid sequence of SEQ ID NO: 6, preferably wherein SEQ ID NO: 6 comprises no amino acid mutations at positions 77, 80, 287, 289, 311, 482, 483, and 669.

In some embodiments, any of the host cells described herein comprise a nucleic acid encoding a carrier protein, e.g., a protein to which the O-antigen polysaccharide(s) produced by the host cell glycosylation machinery can be attached to form a bioconjugate. The host cell can comprise a nucleic acid encoding any carrier protein known to those skilled in the art in view of the present disclosure including, but not limited to, detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

In preferred embodiments, a host cell further comprises a nucleic acid encoding detoxified Exotoxin A of P. aeruginosa (EPA). Preferably, the EPA carrier protein comprises 1-10 glycosylation sites, preferably 2 to 4 glycosylation sites, most preferably 4 glycosylation sites, such as 1-10, preferably 2-4, and more preferably 4 glycosylation sites each comprising a glycosylation consensus sequence having the amino acid sequence of SEQ ID NO: 1, and more preferably having the amino acid sequence of SEQ ID NO: 2. In a specific embodiment, a host cell further comprises a nucleic acid encoding EPA-4 carrier protein comprising SEQ ID NO: 3.

In certain embodiments, the carrier proteins used in the generation of the bioconjugates by the host cells described herein comprise a “tag,” i.e., a sequence of amino acids that allows for the isolation and/or identification of the carrier protein. For example, adding a tag to a carrier protein can be useful in the purification of that protein and, hence, the purification of conjugate vaccines comprising the tagged carrier protein. Exemplary tags that can be used herein include, without limitation, histidine (HIS) tags (e.g., hexa-histidine-tag, or 6×His-Tag), FLAG-TAG, and HA tags. In certain embodiments, the tags used herein are removable, e.g., removal by chemical agents or by enzymatic means, once they are no longer needed, e.g., after the protein has been purified. In other embodiments, the carrier protein does not comprise a tag.

In certain embodiments, the carrier proteins described herein comprise a signal sequence that targets the carrier protein to the periplasmic space of the host cell that expresses the carrier protein. In a specific embodiment, the signal sequence is from E. coli DsbA, E. coli outer membrane porin A (OmpA), E. coli maltose binding protein (MalE), Erwinia carotovorans pectate lyase (PelB), FlgI, NikA, or Bacillus sp. endoxylanase (XynA), heat labile E. coli enterotoxin LTIIb, Bacillus endoxylanase XynA, or E. coli flagellin (FlgI). In one embodiment, the signal sequence comprises SEQ ID NO: 10. A signal sequence may be cleaved off after translocation of the protein to the periplasm and may thus no longer be present in the final carrier protein of a bioconjugate.

In certain embodiments, additional modifications can be introduced (e.g., using recombinant techniques) into the host cells described herein. For example, host cell nucleic acids (e.g., genes) that encode proteins that form part of a possibly competing or interfering glycosylation pathway (e.g., compete or interfere with one or more heterologous genes involved in glycosylation that are recombinantly introduced into the host cell) can be deleted or modified in the host cell background (genome) in a manner that makes them inactive/dysfunctional (i.e., the host cell nucleic acids that are deleted/modified do not encode a functional protein). In certain embodiments, when nucleic acids are deleted from the genome of the host cells provided herein, they are replaced by a desirable sequence, e.g., a sequence that is useful for production of an O antigen polysaccharide or bioconjugate thereof.

Exemplary genes or gene clusters that can be deleted in host cells (and, in some cases, replaced with other desired nucleic acid sequences) include genes or gene clusters of host cells involved in glycolipid biosynthesis, such as waaL (see, e.g., Feldman et al., 2005, PNAS USA 102:3016-3021), the lipid A core biosynthesis cluster (waa), galactose cluster (gal), arabinose cluster (ara), colonic acid cluster (wc), capsular polysaccharide cluster, undecaprenol-p biosynthesis genes (e.g. uppS, uppP), und-P recycling genes, metabolic enzymes involved in nucleotide activated sugar biosynthesis, enterobacterial common antigen cluster (eca), and prophage O antigen modification clusters like the gtrABS cluster or regions thereof. In a specific embodiment, the host cells described herein are modified such that they do not produce any O antigen polysaccharide other than a desired O antigen polysaccharide, e.g., glucosylated O4 antigen polysaccharide.

In a specific embodiment, the waaL gene is deleted or functionally inactivated from the genome of a host cell (e.g., recombinant host cell) provided herein. The terms “waaL” and “waaL gene” refer to the O-antigen ligase gene encoding a membrane bound enzyme with an active site located in the periplasm. The encoded enzyme transfers undecaprenylphosphate (UPP)-bound O antigen to the lipid A core, forming lipopolysaccharide. Deletion or disruption of the endogenous waaL gene (e.g., AwaaL strains) disrupts transfer of the O-antigen to lipid A, and can instead enhance transfer of the O-antigen to another biomolecule, such as a carrier protein.

In another specific embodiment, one or more of the waaL gene, gtrA gene, gtrB gene, gtrS gene, and the rfb gene cluster is deleted or functionally inactivated from the original genome of a prokaryotic host cell provided herein.

In one embodiment, a host cell used herein is E. coli that produces a bioconjugate of glucosylated O4 antigen polysaccharide, wherein the waaL gene is deleted or functionally inactivated from the genome of the host cell, and a gtrS gene specific to E. coli O4 antigen polysaccharide is inserted. In certain embodiments for production strains for bioconjugates of the glucosylated O4 O-antigen, a gtrS gene encoding a glucosyl transferase having at least 80% sequence identity to SEQ ID NO:4 is inserted in the place of a gtrS gene of the parent strain, so as to replace the gtrS gene in that parent strain with the one that is responsible for glucosylation of the O4 antigen. An example of such a parent strain is E. coli K-12 strain W3110. The gtrA and gtrB genes can be homologous to the parent strain, or alternatively one or both of these genes can be heterologous to the parent strain. Typically, and unlike the gtrS gene, these gtrA and gtrB genes are not specific for the O-antigen structure.

Also described herein are methods of making recombinant host cells. Recombinant host cells produced by the methods described herein can be used to produce bioconjugates of E. coli O antigens. The methods comprise introducing one or more recombinant nucleic acid molecules into a cell to produce the recombinant host cell. Typically, the recombinant nucleic acid molecules are heterologous. Any method known in the art in view of the present disclosure can be used to introduce recombinant nucleic acid molecules into a host cell. Recombinant nucleic acids can be introduced into the host cells described herein using any methods known to those of ordinary skill in the art, e.g., electroporation, chemical transformation, by heat shock, natural transformation, phage transduction, and conjugation. In specific embodiments, recombinant nucleic acids are introduced into the host cells described herein using a plasmid. For example, the heterologous nucleic acids can be expressed in the host cells by a plasmid (e.g., an expression vector). In another specific embodiment, heterologous nucleic acids are introduced into the host cells described herein using the method of insertion into the genome as for instance described in WO 2014/037585, WO 2014/057109, or WO 2015/052344.

E. coli strains that are used routinely in molecular biology as both a tool and a model organism can for instance be used as parents for host cells in certain embodiments. Non-limiting examples include E. coli K12 strains (for example, such as W1485, W2637, W3110, MG1655, DH1, DH5a, DH10, etc.), B strains (e.g. BL-21, REL606, etc.), C strains, or W strains. In one particular embodiment, the host strain is derived from parent strain W3110. This strain can for instance be obtained from the E. coli Genetic Stock Center at Yale. For more information on E. coli, see e.g. Ecoliwiki.net.

In some embodiments, the host cells described herein can be used to produce bioconjugates comprising an E. coli O antigen polysaccharide covalently linked to a carrier protein. Methods of producing such bioconjugates using host cells are known in the art. See, e.g., WO 2003/074687 and WO 2006/119987. Such methods comprise culturing any of the recombinant host cells described herein under conditions for production of the bioconjugate. Bioconjugates can be isolated, separated, and/or purified from recombinant host cells using any method known in the art in view of the present disclosure. For example, bioconjugates can be purified by any method known in the art for purification of a protein, for instance, by chromatography (e.g., ion exchange, anionic exchange, affinity, and sizing column chromatography), centrifugation, differential solubility, or by any other standard technique for the purification of proteins. See, e.g., methods described in WO 2009/104074. Further, the bioconjugates can be fused to heterologous polypeptide sequences to facilitate purification. The actual conditions used to purify a particular bioconjugate will depend, in part, on factors such as net charge, hydrophobicity, and/or hydrophilicity of the bioconjugate, and will be apparent to those skilled in the art. Preparation of bioconjugates for O1A, O2, O6A, and O25B, as well as vaccine compositions comprising these, have for instance been described in WO 2015/124769 and in WO 2017/035181.

Also provided are bioconjugates produced by the methods described herein, i.e., using the recombinant host cells described herein.

In some embodiments, a method of preparing a bioconjugate of an E. coli O-antigen polysaccharide covalently linked to a carrier protein comprises: (i) providing a recombinant host cell comprising (a) nucleotide sequence of an rfb gene cluster for the O-antigen polysaccharide; (b) a nucleotide sequence encoding a carrier protein, preferably EPA, comprising at least one glycosylation site comprising a glycosylation consensus sequence having SEQ ID NO: 1, preferably SEQ ID NO: 2, and more preferably comprising four glycosylation sites each comprising a glycosylation consensus sequence having SEQ ID NO: 2; and (c) nucleotide sequence encoding an oligosaccharyl transferase, for instance PglB oligosaccharyl transferase or variant thereof.

In certain embodiments, E. coli O-antigen polysaccharides are covalently bound to the carrier protein at a particular polysaccharide to protein ratio by weight (w/w). This ratio of amount of O-antigen polysaccharide by weight covalently bound to the carrier protein by weight is referred to as the “glycan/protein ratio” or “polysaccharide/protein ratio” or “PS/protein ratio”. In some embodiments, the O-antigen polysaccharide is covalently bound to the carrier protein at a polysaccharide to protein (w/w) ratio of about 1:20 to 20:1, preferably 1:10 to 10:1, more preferably 1:3 to 3:1. In certain non-limiting embodiments for bioconjugates described herein, glycan/protein ratio is about 0.1 to 0.5, such as 0.1, 0.15, 0.2, 0.25, 0.3, 0.35, 0.4, 0.45, or 0.5. In such embodiments, the weight ratio of the O-antigen polysaccharide: protein is about 1:10 to 1:2, such as 1:10: 1:9: 1:8, 1:7, 1:6, 1:5, 1:4, 1:3, or 1:2, depending on the particular O-antigen serotype. In certain embodiments the glycan/protein ratio is from about 0.15 to about 0.45. In general, a higher glycan/protein ratio of O-antigen polysaccharide to carrier protein is preferred, because a high amount of carrier protein can lead to immunological interference in some instances. Also, a higher glycan/protein ratio would help getting sufficient O-antigen polysaccharide dosed in the form of bioconjugate, while keeping the amount of carrier protein relatively low, which is especially beneficial for multivalent compositions where multiple serotypes are to be covered by the composition, e.g. compositions comprising bioconjugates from at least 4 different O-antigens, at least 5 different O-antigens, at least 6 different O-antigens, at least 7 different O-antigens, at least 8 different O-antigens, at least 9 different O-antigens, at least 10 different O-antigens, etc.

A glycan/protein ratio of a conjugate according to the invention can be determined by determining the protein amount and the glycan amount. Protein amount can be determined by measurement of UV absorbance at 280 nm (A280). Glycan amount can be determined based on ion chromatography with pulsed amperometric detection (IC-PAD) of a sugar in the repeat unit (e.g. of Man for O8 in Table 1, and of GlcNAc for the other glycans in Table 1), after which the structural information of the repeat unit can be used to calculate the total glycan amount (e.g. the repeat unit of O1A has a molar mass of 845 Da and one mole of such a repeat unit contains one mole of GlcNAc, enabling calculation of the total glycan amount when the amount of GlcNAc has been determined by IC-PAD).

In some embodiments, a bioconjugate of an E. coli O25B antigen polysaccharide covalently linked to a carrier protein as described herein has a certain degree of acetylation at position 2 of the L-Rh sugar. The degree of O-acetylation of O25B antigen polysaccharide in a (bio)conjugate is preferably at least 30%, preferably at least 50%, such as at least 50%, 55%, 60%, 65%, 70%, 75%, 80%, 85%, 90%, 95%, or 100%.

Similarly, the degree of O-acetylation of an E. coli O16 antigen polysaccharide in a (bio)conjugate is preferably at least 30%, preferably at least 50%, such as at least 50%, 55%, 60%, 65%, 70%, 75%, 80%, 85%, 90%, 95%, or 100%.

In specific embodiments, a method of preparing a bioconjugate of an O-antigen polysaccharide comprises providing a recombinant host cell comprising nucleic acid sequence encoding a particular oligosaccharyl transferase enzyme, particularly a PglB oligosaccharyl transferase or variant thereof, depending on the O-antigen polysaccharide bioconjugate to be produced. The particular oligosaccharyl transferase enzyme variant may impact the yield of bioconjugate produced by the host cell. Typically, a higher yield is preferred, since the yield will impact the costs for producing a specific bioconjugate, which is especially important for multivalent compositions comprising several different bioconjugates.

In one particular embodiment, when the O-antigen is O75 antigen polysaccharide, the PglB oligosaccharyl transferase comprises the amino acid mutation of N311V, wherein the amino acid mutations are relative to the wild-type PglB having the amino acid sequence of SEQ ID NO: 6.

In another particular embodiment, when the O-antigen is O1A, O6A, or O15 antigen polysaccharide, the PglB oligosaccharyl transferase comprises the amino acid mutations of N311V, K482R, D483H, and A669V, wherein the amino acid mutations are relative to the wild-type PglB having the amino acid sequence of SEQ ID NO: 6.

In another particular embodiment, when the O-antigen is glucosylated O4 antigen polysaccharide, the PglB oligosaccharyl transferase comprises the amino acid mutation N311V, or the amino acid mutations of Y77H and N311V, wherein the amino acid mutations are relative to the wild-type PglB having the amino acid sequence of SEQ ID NO: 6.

In another particular, embodiment, when the O-antigen is O16 antigen polysaccharide, the PglB oligosaccharyl transferase comprises the amino acid mutations of Y77H, S80R, Q287P, K289R, and N311V, wherein the amino acid mutations are relative to the wild-type PglB having the amino acid sequence of SEQ ID NO: 6.

In another particular embodiment, when the O-antigen is O8, O18A, O25B, or O2 antigen polysaccharide, the PglB oligosaccharyl transferase comprises the amino acid sequence of SEQ ID NO: 6, wherein SEQ ID NO: 6 comprises no amino acid mutations at positions 77, 80, 287, 289, 311, 482, 483, and 669. In certain embodiments thereof, the PglB oligosaccharyl transferase comprises the amino acid sequence of SEQ ID NO: 6.

In some embodiments, bioconjugates of O-antigen polysaccharides produced by recombinant host cells encoding the oligosaccharyl transferase enzymes per the O-antigen/PglB oligosaccharyl transferase pairings indicated above preferably have one or more of the preferred attributes described herein, e.g., glycan/protein ratio and/or percent of multi-glycosylated carrier protein.

### Compositions and Combinations

Provided herein are compositions and combinations comprising E. coli O75 and O6 antigen polysaccharides, wherein each of the antigen polysaccharides is independently covalently linked to a carrier protein, and optionally further comprising one or more, preferably all, of E. coli O1, O2, O4, O15, O16, O18, O25 antigen polysaccharides, wherein each of the antigen polysaccharides is independently covalently linked to a carrier protein. A combination of O-antigen polysaccharides or conjugates, e.g., bioconjugates, can comprise multiple compositions, but it is preferred if a combination of O-antigen polysaccharides or conjugates, e.g., bioconjugates, is present in the same composition.

The compositions and combinations described herein are useful in the treatment and prevention of infection of subjects (e.g., human subjects) with E. coli, preferably prevention of invasive ExPEC disease. In some embodiments, a composition is an immunogenic composition. As used herein, an “immunogenic composition” refers to a composition that can elicit an immune response in a host or subject to whom the composition is administered. Compositions and immunogenic compositions can further comprise a pharmaceutically acceptable carrier. As used herein, the term “pharmaceutically acceptable” means approved by a regulatory agency of a Federal or a state government or listed in the U.S. Pharmacopeia or other generally recognized pharmacopeia for use in animals, and more particularly in humans. The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin.

In one embodiment, a composition of the invention comprises the (bio)conjugates as described herein in a Tris-buffered saline (TBS) pH 7.4 (e.g. containing Tris, NaCl and KCl, e.g. at 25 mM, 137 mM and 2.7 mM, respectively). In other embodiments, the compositions of the invention comprise (bio)conjugates as described herein in about 10 mM KH₂PO₄/Na₂HPO₄ buffer at pH of about 7.0, about 5% (w/v) sorbitol, about 10 mM methionine, and about 0.02% (w/v) polysorbate 80. In other embodiments, the compositions of the invention comprise (bio)conjugates as described herein in about 10 mM KH₂PO₄/Na₂HPO₄ buffer at pH of about 7.0, about 8% (w/v) sucrose, about 1 mM EDTA, and about 0.02% (w/v) polysorbate 80 (see e.g. WO 2018/077853 for suitable buffers for bioconjugates of E. coli O-antigens covalently bound to EPA carrier protein). In other embodiments, the compositions of the invention comprise (bio)conjugates as described herein in about 5 mM succinate/0.9% NaCl, pH 6.0.

Provided herein are compositions (e.g., pharmaceutical and/or immunogenic compositions) that are multivalent compositions, e.g., bivalent, trivalent, tetravalent, etc. compositions. For example, a multivalent composition comprises more than one antigen, such as an E. coli O-antigen, glycoconjugate, or bioconjugate thereof. In particular embodiments, multivalent compositions provided herein comprise a bioconjugate of an E. coli O75 antigen polysaccharide and a bioconjugate of an E. coli O6A antigen polysaccharide. In some embodiments, multivalent compositions provided herein comprise at least one additional antigen or bioconjugate.

Typically the compositions of the invention can be prepared by first obtaining individual glycoconjugates for each of the E. coli O-antigen polysaccharides as described herein by independently covalently linking these O-antigen polysaccharides to a carrier protein e.g. by chemical conjugation or bioconjugation, and subsequently mixing the individual glycoconjugates in amounts and ratios as described herein to obtain compositions according to the invention.

In one embodiment, a composition (e.g., pharmaceutical and/or immunogenic composition) is a multivalent composition comprising an E. coli O75 antigen polysaccharide covalently linked to a carrier protein as described herein, and at least one additional antigen.

In some embodiments, the additional antigen is antigen saccharide or polysaccharide, more preferably an E. coli O-antigen polysaccharide, such as E. coli O-antigens of one or more of the O1, O2, O4, O6, O8, O15, O16, O18, and O25 serotypes and subserotypes thereof, preferably each individually conjugated to a carrier protein, wherein the carrier protein for each serotype may be the same or may differ between some or all serotypes. Preferably, a multivalent composition comprising a (bio)conjugate of an E. coli O75 polysaccharide further comprises E. coli O-antigens of each of the O1, O2, O4, O6, O15, O16, O18, and O25 serotypes or subserotypes thereof. In some embodiments, each of the additional E. coli O-antigen polysaccharides is a glycoconjugate, meaning that the E. coli O-antigen polysaccharide is covalently linked to another chemical species, e.g., protein, peptide, lipid, etc., most preferably a carrier protein, such as by chemical or enzymatic methods. In preferred embodiments, each of the additional E. coli O-antigen polysaccharides is a bioconjugate in which the O-antigen polysaccharide is covalently linked to, e.g. a carrier protein, via a glycosidic bond enzymatically by host cell machinery. In certain embodiments, the multivalent composition further comprises E. coli O-antigen of the O8 serotype, preferably in the form of a glycoconjugate, preferably a bioconjugate. Compositions provided herein in certain embodiments can comprise 1-20 additional glycoconjugates, more preferably bioconjugates of E. coli O-antigen polysaccharides, such as 1, 2, 3, 4, 5, 6, 7, 8, 9, 10, 11, 12, 13, 14, 15, 16, 17, 18, 19 or 20 additional glycoconjugates or preferably bioconjugates of E. coli O-antigen polysaccharides. Other antigens can be included in the compositions provided herein, such as peptide, protein, or lipid antigens, etc.

In some embodiments, a composition (e.g., pharmaceutical and/or immunogenic composition) comprises a (bio)conjugate of an E. coli O75 antigen polysaccharide and a (bio)conjugate of an E. coli O6 antigen polysaccharide, and at least one additional antigen polysaccharide selected from the group consisting of an E. coli O1 antigen polysaccharide, E. coli O2 antigen polysaccharide, E. coli glucosylated O4 antigen polysaccharide, E. coli O8 antigen polysaccharide, E. coli O15 antigen polysaccharide, E. coli O16 antigen polysaccharide, E. coli O18 antigen polysaccharide, and E. coli O25 antigen polysaccharide, preferably at least E. coli O1 antigen polysaccharide, E. coli O2 antigen polysaccharide and E. coli O25 antigen polysaccharide, more preferably at least E. coli O1 antigen polysaccharide, E. coli O2 antigen polysaccharide, E. coli O25 antigen polysaccharide, E. coli O4 antigen polysaccharide, E. coli O15 antigen polysaccharide, E. coli O16 antigen polysaccharide and E. coli O18 antigen polysaccharide. Preferably the O1 antigen polysaccharide is an O1A antigen polysaccharide, the O6 antigen polysaccharide is an O6A antigen polysaccharide, the O4 antigen polysaccharide is a glucosylated O4 antigen polysaccharide, the O18 antigen polysaccharide is an O18A antigen polysaccharide, and the O25 antigen polysaccharide is an O25B antigen polysaccharide. Preferably, each of the additional O-antigen polysaccharides is covalently linked to a carrier protein, and is more preferably a bioconjugate.

In some embodiments, a composition comprises E. coli O75 and at least one of E. coli O1, O2, or O6 antigen polysaccharides, preferably at least one of E. coli O1 or O6 antigen polysaccharides, wherein each of the antigen polysaccharides is independently covalently linked to a carrier protein, and wherein the ratio of concentrations of O75 antigen polysaccharide to O1, 02, and/or O6 antigen polysaccharide is about 1.2:1 to about 8:1, preferably about 1.5:1 to about 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1. In preferred embodiments, the composition further comprises E. coli O25 antigen polysaccharide, preferably E. coli O25B antigen polysaccharide, covalently linked to a carrier protein, and wherein the ratio of concentrations of O25 antigen polysaccharide to O1, O2, and/or O6 antigen polysaccharide is about 1.5:1 to about 4:1, preferably about 2:1. In such embodiments, preferably the ratio of concentrations of O25 antigen polysaccharide to O75 antigen polysaccharide is about 2:1 to about 1:1, preferably about 1.5:1, more preferably about 1:1. In certain embodiments thereof, the ratio of concentrations of O25 antigen polysaccharide to O2 antigen polysaccharide is about 4:1 to about 2:1. In certain embodiments, the composition comprises E. coli O75, O1, O2, O6 and O25 antigen polysaccharides, preferably wherein the O1, 06 and O25 antigen polysaccharides respectively are O1A, O6A and O25B polysaccharides, wherein each of the antigen polysaccharides is independently covalently linked to a carrier protein, and wherein the ratio of concentrations of antigen polysaccharides O75:O1:O2:O6:O25 is about 2:1:1:1:2. In certain embodiments, the O-antigens are covalently linked to a carrier protein by bioconjugation, e.g. via N-links to Asn-residues in the carrier protein. In certain embodiments, the concentration of O75 antigen polysaccharide in the composition is about 8-64 μg/mL, preferably about 16-32 μg/mL, preferably about 28-36 μg/mL, e.g. about 32 μg/mL. In certain embodiments, the composition further comprises one or more, preferably all, of E. coli O4, O15, O16, O18 antigen polysaccharides, wherein each of the antigen polysaccharides is independently covalently linked to a carrier protein, preferably wherein the O4 is glucosylated, and the O18 antigen is O18A. In certain embodiments, the composition further comprises E. coli O8 antigen polysaccharide covalently linked to a carrier protein. In certain embodiments, the ratio of concentrations of antigen polysaccharides O75:O4:O15:O16:O18:O8 in as far as each of these is present in the composition is about 2:1:1:1:1:1. In additional embodiments, the composition can comprise one or more further E. coli O antigen polysaccharides, wherein each of the antigen polysaccharides is independently covalently linked to a carrier protein.

In one embodiment, an O1 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O1 antigen polysaccharide comprises the structure of formula (O1A) as shown in Table 1, wherein n is an integer of 1-100, preferably 3-50, e.g. 5-40, preferably 5-30, e.g. 7 to 25, e.g. 10 to 20. Preferably, the O1 antigen polysaccharide is part of a bioconjugate and is covalently linked to a carrier protein, e.g., EPA or CRM₁₉₇. In certain embodiments the weight ratio between O75:O1 antigen polysaccharides in the composition and/or as administered to a subject is between about 1.2:1 and 8:1, preferably between about 1.5:1 and 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1.

In one embodiment, an O2 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O2 antigen polysaccharide comprises the structure of formula (O2) as shown in Table 1, wherein n is an integer of 1-100, preferably 3-50, e.g. 5-40, preferably 5-30, e.g. 7 to 25, e.g. 10 to 20. Preferably, the O2 antigen polysaccharide is part of a bioconjugate and is covalently linked to a carrier protein, e.g., EPA or CRM₁₉₇. In certain embodiments the weight ratio between O75:O2 antigen polysaccharides in the composition and/or as administered to a subject is between about 1.2:1 and 8:1, preferably between about 1.5:1 and 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1.

In one embodiment, an O4 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O4 antigen polysaccharide is a glucosylated O4 antigen polysaccharide, and in a specific embodiment comprises the structure of formula (O4-Glc+) as shown in Table 1, wherein n is an integer of 1-100, preferably 3-50, e.g. 5-40, preferably 5-30, e.g. 7 to 25, e.g. 10 to 20. Preferably, the O4 antigen polysaccharide is part of a bioconjugate and is covalently linked to a carrier protein, e.g., EPA or CRM₁₉₇. In certain embodiments the weight ratio between O75:O4 antigen polysaccharides in the composition and/or as administered to a subject is between about 1.2:1 and 8:1, preferably between about 1.5:1 and 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1.

In one embodiment, an O6 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O6 antigen polysaccharide comprises the structure of formula (O6A) as shown in Table 1, wherein n is an integer of 1-100, preferably 3-50, e.g. 5-40, preferably 5-30, e.g. 7 to 25, e.g. 10 to 20. Preferably, the O6 antigen polysaccharide is part of a bioconjugate and is covalently linked to a carrier protein, e.g., EPA or CRM₁₉₇. In certain embodiments the weight ratio between O75:O6 antigen polysaccharides in the composition and/or as administered to a subject is between about 1.2:1 and 8:1, preferably between about 1.5:1 and 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1.

In one embodiment, an O8 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O8 antigen polysaccharide comprises the structure of formula (O8) as shown in Table 1, wherein n is an integer of 1-100, preferably 3-50, e.g. 5-40, preferably 5-30, e.g. 7 to 25, e.g. 10 to 20. Preferably, the O8 antigen polysaccharide is part of a bioconjugate and is covalently linked to a carrier protein, e.g., EPA or CRM₁₉₇. In certain embodiments the weight ratio between O75:O8 antigen polysaccharides in the composition and/or as administered to a subject is between about 1.2:1 and 8:1, preferably between about 1.5:1 and 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1.

In one embodiment, an O15 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O15 antigen polysaccharide comprises the structure of formula (O15) as shown in Table 1, wherein n is an integer of 1-100, preferably 3-50, e.g. 5-40, preferably 5-30, e.g. 7 to 25, e.g. 10 to 20. Preferably, the O15 antigen polysaccharide is part of a bioconjugate and is covalently linked to a carrier protein, e.g., EPA or CRM₁₉₇. In certain embodiments the weight ratio between O75:O15 antigen polysaccharides in the composition and/or as administered to a subject is between about 1.2:1 and 8:1, preferably between about 1.5:1 and 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1.

In one embodiment, an O16 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O16 antigen polysaccharide comprises the structure of formula (O16) as shown in Table 1, wherein n is an integer of 1-100, preferably 3-50, e.g. 5-40, preferably 5-30, e.g. 7 to 25, e.g. 10 to 20. Preferably, the O16 antigen polysaccharide is part of a bioconjugate and is covalently linked to a carrier protein, e.g., EPA or CRM₁₉₇. In certain embodiments the weight ratio between O75:O16 antigen polysaccharides in the composition and/or as administered to a subject is between about 1.2:1 and 8:1, preferably between about 1.5:1 and 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1.

In one embodiment, an O18 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O18 antigen polysaccharide comprises the structure of formula (O18A) as shown in Table 1, wherein n is an integer of 1-100, preferably 3-50, e.g. 5-40, preferably 5-30, e.g. 7 to 25, e.g. 10 to 20. Preferably, the O18 antigen polysaccharide is part of a bioconjugate and is covalently linked to a carrier protein, e.g., EPA or CRM₁₉₇. In certain embodiments the weight ratio between O75:O18 antigen polysaccharides in the composition and/or as administered to a subject is between about 1.2:1 and 8:1, preferably between about 1.5:1 and 4:1, more preferably about 2:1, e.g. about 1.5:1, 1.6:1, 1.7:1, 1.8:1, 1.9:1, 2.0:1, 2.1:1, 2.2:1, 2.3:1, 2.4:1, or 2.5:1.

In one embodiment, an O25 antigen polysaccharide (e.g., in isolated form or as part of a glycoconjugate or bioconjugate) is used in a composition provided herein (e.g., in combination with an O75 antigen polysaccharide or bioconjugate thereof). In a specific embodiment, the O25 antigen polysaccharide comprises an O25B antigen polysaccharide, and in a specific embodiment comprises the structure of formula (O25B) as shown in Table 1, wherein n is an integer of 1-100, preferably 3-50, e.g. 5-40, preferably 5-30, e.g. 7 to 25, e.g. 10 to 20. Preferably, the O25 antigen polysaccharide is part of a bioconjugate and is covalently linked to a carrier protein, e.g., EPA or CRM₁₉₇. In certain embodiments the weight ratio between O75:O25 antigen polysaccharides in the composition and/or as administered to a subject is between about 1:4 and 1:0.5, preferably between about 1:2 and 1:1, more preferably about 1:1, e.g. about 1:1.5, 1:1.4, 1:1.3, 1:1.2, 1:1.1, 1:1.0, 1:0.9, 1:0.8, 1:0.7, 1:0.6, or 1:0.5.

In another embodiment, a composition (e.g., a pharmaceutical and/or immunogenic composition) comprises at least the E. coli O75, O1, O2, O4, O6 and O25 antigen polysaccharides conjugated to carrier protein, preferably bioconjugates of the O75, O1A, O2, glucosylated O4, O6A and O25B antigen polysaccharides covalently linked to a carrier protein, e.g., EPA or CRM₁₉₇ (i.e., an at least hexavalent composition). In one embodiment the weight ratio between O75:O1:O2:O4:O6:O25 antigen polysaccharides in the composition and/or as administered to a subject is about 2:1:1:1:1:2.

In a preferred embodiment, a composition (e.g., a pharmaceutical and/or immunogenic composition) comprises at least the E. coli O1, O2, O4, O6, O15, O16, O18, O25 and O75 antigen polysaccharides conjugated to carrier protein, preferably bioconjugates of the O1A, O2, glucosylated O4, O6A, O15, O16, O18A, O25B and O75 antigen polysaccharides covalently linked to a carrier protein, e.g., EPA or CRM₁₉₇ (i.e., an at least 9-valent composition). In one embodiment the weight ratio between O75:O1:O2:O4:O6:O15:O16:O18:O25 antigen polysaccharides in the composition and/or as administered to a subject is about 2:1:1:1:1:1:1:1:2.

In another preferred embodiment, a composition (e.g., a pharmaceutical and/or immunogenic composition) comprises at least the E. coli O1, O2, O4, O6, O8, O15, O16, O18, O25 and O75 antigen polysaccharides conjugated to carrier protein, preferably bioconjugates of the O1A, O2, glucosylated O4, O6A, O8, O15, O16, O18A, O25B and O75 antigen polysaccharides covalently linked to a carrier protein, e.g., EPA or CRM₁₉₇ (i.e., an at least 10-valent composition). In one embodiment the weight ratio between O75:O1:O2:O4:O6:O8:O15:O16:O18:O25 antigen polysaccharides in the composition and/or as administered to a subject is about 2:1:1:1:1:1:1:1:1:2.

Also contemplated herein are compositions which optionally further comprise additional O-antigens (e.g., in isolated form, or as part of a glycoconjugate or bioconjugate) from other E. coli serotypes. In some embodiments, a composition (e.g., a pharmaceutical and/or immunogenic composition) comprises at least the 6-, 9- or 10-valent compositions as described above, and further comprises from 1 to 15 additional E. coli antigen polysaccharides, preferably (bio)conjugates of the antigen polysaccharides covalently linked to a carrier protein, e.g., EPA or CRM₁₉₇, such as 1, 2, 3, 4, 5, 6, 7, 8, 9, 10, 11, 12, 13, 14, or 15 additional E. coli antigen polysaccharides. In certain embodiments, the 6-, 9- or 10-valent compositions as described above do not comprise additional E. coli antigen polysaccharides or conjugates thereof, i.e. such compositions include 6, 9, or 10 but not more E. coli antigen polysaccharides, respectively, preferably in the form of conjugates, preferably bioconjugates. It is thus an embodiment to provide a composition that comprises 9 conjugates, in particular E. coli O1, O2, O4, O6, O15, O16, O18, O25 and O75 antigen polysaccharides conjugated to carrier protein, preferably bioconjugates of the O1A, O2, glucosylated O4, O6A, O15, O16, O18A, O25B and O75 antigen polysaccharides covalently linked to a carrier protein, e.g., EPA or CRM₁₉₇ (i.e., a 9-valent composition), and no additional conjugates of E. coli O-antigen polysaccharides covalently linked to a carrier protein. It is thus another embodiment to provide a composition that comprises 10 conjugates, in particular E. coli O1, O2, O4, O6, O8, O15, O16, O18, O25 and O75 antigen polysaccharides conjugated to carrier protein, preferably bioconjugates of the O1A, O2, glucosylated O4, O6A, O8, O15, O16, O18A, O25B and O75 antigen polysaccharides covalently linked to a carrier protein, e.g., EPA or CRM₁₉₇ (i.e., a 10-valent composition), and no additional conjugates of E. coli O-antigen polysaccharides covalently linked to a carrier protein.

In some preferred embodiments, each of the additional E. coli O1, O2, O4, O6, O15, O16, O18, and/or O25 antigen polysaccharides is covalently linked to a carrier protein. The O-antigen polysaccharide can be linked to a carrier protein by chemical or other synthetic methods, or the O-antigen polysaccharide can be part of a bioconjugate, and is preferably part of a bioconjugate. Preferably the O-antigens per serotype covered by the composition are each separately coupled to a carrier protein, i.e., each glycoconjugate or bioconjugate covering a specific serotype can be produced separately before being mixed in a composition according to the invention. The carrier protein preferably can be the same for each glycoconjugate or bioconjugate in the composition, or alternatively can differ for some or all of the E. coli O-antigens, e.g. O-antigens of different serotypes can also be conjugated to different carrier proteins. Any carrier protein known to those skilled in the art in view of the present disclosure can be used. Suitable carrier proteins include, but are not limited to, detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae. Preferably, the carrier protein is EPA or CRM₁₉₇.

In some embodiments, each of the additional E. coli O1(A), O2, (glucosylated) O4, O6(A), O15, O16, O18(A), and/or O25(B) antigen polysaccharides, particularly when part of a bioconjugate, is covalently linked to an asparagine (Asn) residue in the carrier protein, wherein the Asn residue is present in a glycosylation site comprising a glycosylation consensus sequence Asn-X-Ser(Thr), wherein X can be any amino acid except Pro (SEQ ID NO: 1), preferably wherein the Asn residue is present in a glycosylation site comprising a glycosylation consensus sequence Asp(Glu)-X-Asn-Z-Ser(Thr), wherein X and Z are independently selected from any amino acid except Pro (SEQ ID NO: 2). The carrier protein can comprise 1-10 glycosylation sites, preferably 2 to 4 glycosylation sites, most preferably 4 glycosylation sites, each comprising a glycosylation consensus sequence. In a particular embodiment, the carrier protein is EPA-4 carrier protein, for instance EPA-4 carrier protein comprising the amino acid sequence of SEQ ID NO: 3.

In a particular embodiment, provided herein is a composition (e.g., pharmaceutical and/or immunogenic composition) comprising: (i) a bioconjugate of an E. coli O75 antigen polysaccharide covalently linked to a detoxified Exotoxin A of P. aeruginosa carrier protein comprising SEQ ID NO: 3 (EPA-4 carrier protein), wherein the E. coli O75 antigen polysaccharide comprises the structure of Formula (O75); (ii) a bioconjugate of an E. coli O1A antigen polysaccharide covalently linked to an EPA-4 carrier protein, wherein the E. coli O1A antigen polysaccharide comprises the structure of Formula (O1A); (iii) a bioconjugate of an E. coli O2 antigen polysaccharide covalently linked to an EPA-4 carrier protein, wherein the E. coli O2 antigen polysaccharide comprises the structure of Formula (O2); (iv) a bioconjugate of an E. coli O6A antigen polysaccharide covalently linked to an EPA-4 carrier protein, wherein the E. coli O6A antigen polysaccharide comprises the structure of Formula (O6A); (v) a bioconjugate of an E. coli O15 antigen polysaccharide covalently linked to an EPA-4 carrier protein, wherein the E. coli O15 antigen polysaccharide comprises the structure of Formula (O15); (vi) a bioconjugate of an E. coli O16 antigen polysaccharide covalently linked to an EPA-4 carrier protein, wherein the E. coli O16 antigen polysaccharide comprises the structure of Formula (O16); (vii) a bioconjugate of an E. coli O18A antigen polysaccharide covalently linked to an EPA-4 carrier protein, wherein the E. coli O18A antigen polysaccharide comprises the structure of Formula (O18A); (viii) a bioconjugate of an E. coli O25B antigen polysaccharide covalently linked to an EPA-4 carrier protein, wherein the E. coli O25B antigen polysaccharide comprises the structure of Formula (O25B); and (ix) a bioconjugate of an E. coli glucosylated O4 antigen polysaccharide covalently linked to an EPA-4 carrier protein, wherein the E. coli glucosylated O4 antigen polysaccharide comprises the structure of Formula (O4-Glc+), wherein each of the Formulas is provided in Table 1, and for each of the Formulas independently n is an integer of 1 to 100, e.g. 1 to 50, preferably 3 to 50, e.g. 5 to 40, preferably 5-30, e.g. 7 to 25, e.g. 10 to 20.

In a particular embodiment, said composition (e.g. pharmaceutical and/or immunogenic composition) further comprises: (x) a bioconjugate of an E. coli O8 antigen polysaccharide covalently linked to an EPA-4 carrier protein, wherein the E. coli O8 antigen polysaccharide comprises the structure of Formula (O8) as shown in Table 1, wherein n for this structure is an integer of 1 to 100, e.g. 1 to 50, preferably 3 to 50, e.g. 5 to 40, preferably 5-30, e.g. 7 to 25, e.g. 10 to 20.

In some embodiments, a composition provided herein comprises a conjugate of an E. coli O75 antigen polysaccharide, and at least a conjugate of an E. coli O6 antigen polysaccharide, preferably O6A antigen polysaccharide, wherein the conjugate of the E. coli O75 antigen polysaccharide is present in the composition at a concentration that is about 1.2 to 8 times, e.g. about 2 to 4 times higher, such as 1.3, 1.4, 1.5, 1.6, 1.7, 1.8, 1.9, 2.0, 2.1, 2.2, 2.3, 2.4, 2.5, 3, 4, 5, 6, 7, or 8 times higher than the concentration of the O6 antigen polysaccharide present in the composition (all concentrations based on weight of the O-antigen polysaccharides). Preferably the E. coli O75 antigen polysaccharide in the composition is present at a concentration that is about 1.5 to 2.5 times, such as 1.5, 1.6, 1.7, 1.8, 1.9, 2.0, 2.1, 2.2, 2.3, 2.4, or 2.5 times, the concentration of the O6 antigen polysaccharide in the composition.

In particular embodiments, a composition comprises bioconjugates of E. coli O1A, O2, glucosylated O4, O6A, O15, O16, O18, O25B, and O75 antigen polysaccharides, wherein the bioconjugates of O1A:O2:glucosylated O4:O6A:O15:O16:O18:O25B:O75 are present in a ratio (by weight of O-antigen polysaccharide) of 1:1:1:1:1:1:1:2:2, 1:1:1:1:1:1:1:2:1.5, 2:1:1:2:1:1:1:4:3, 2:1:1:2:1:1:1:4:4, 2:1:2:2:2:2:2:4:4, preferably about 1:1:1:1:1:1:1:2:2.

In particular embodiments, a composition comprises bioconjugates of E. coli O1A, O2, glucosylated O4, O6A, O8, O15, O16, O18A, O25B, and O75 antigen polysaccharides, wherein the bioconjugates of O1A:O2:glucosylated O4:O6A:O8:O15:O16:O18A:O25B:O75 are present in a ratio (by weight of O-antigen polysaccharide) of 1:1:1:1:1:1:1:1:2:2, 2:1:2:2:2:2:2:2:4:4, 2:1:1:2:1:1:1:1:4:3, or 2:1:1:2:1:1:1:1:4:4, preferably about 1:1:1:1:1:1:1:1:2:2.

In some embodiments, a composition provided herein comprises a bioconjugate of an E. coli O75 antigen polysaccharide, and at least a bioconjugate of an E. coli O6 antigen polysaccharide, wherein the bioconjugate of the E. coli O75 antigen polysaccharide is present in the composition at a concentration of about 8 to about 50 μg/mL, preferably about 12 to about 40 μg/mL, more preferably about 16 to about 32 μg/mL, such as 16, 18, 20, 22, 24, 26, 28, 30, or 32 μg/mL, preferably about 32 μg/mL. The concentration of the bioconjugate of the E. coli O75 antigen polysaccharide is preferably about 1.2 to about 8 times, e.g., about 2 to 4 times higher, such as 1.5, 2, 3, 4, 5, 6, 7, or 8 times higher than the concentration of the O6 bioconjugate present in the composition.

In one particular embodiment, a pharmaceutical composition is provided, which composition comprises:
(i) a bioconjugate of an E. coli O1A antigen polysaccharide covalently linked to an EPA-4 carrier protein, at a polysaccharide concentration of about 12 to 20, preferably about 16 μg/mL;
(ii) a bioconjugate of an E. coli O2 antigen polysaccharide covalently linked to an EPA-4 carrier protein, at a polysaccharide concentration of about 12 to 20, preferably about 16 μg/mL;
(iii) a bioconjugate of an E. coli O4 antigen polysaccharide covalently linked to an EPA-4 carrier protein, at a polysaccharide concentration of about 12 to 20, preferably about 16 μg/mL;
(iv) a bioconjugate of an E. coli O6A antigen polysaccharide covalently linked to an EPA-4 carrier protein, at a polysaccharide concentration of about 12 to 20, preferably about 16 μg/mL;
(v) a bioconjugate of an E. coli O15 antigen polysaccharide covalently linked to an EPA-4 carrier protein, at a polysaccharide concentration of about 12 to 20, preferably about 16 μg/mL;
(vi) a bioconjugate of an E. coli O16 antigen polysaccharide covalently linked to an EPA-4 carrier protein, at a polysaccharide concentration of about 12 to 20, preferably about 16 μg/mL;
(vii) a bioconjugate of an E. coli O18A antigen polysaccharide covalently linked to an EPA-4 carrier protein, at a polysaccharide concentration of about 12 to 20, preferably about 16 μg/mL;
(viii) a bioconjugate of an E. coli O25B antigen polysaccharide covalently linked to an EPA-4 carrier protein, at a polysaccharide concentration of about 28 to 36, preferably about 32 μg/mL; and
(ix) a bioconjugate of an E. coli O75 antigen polysaccharide covalently linked to an EPA-4 carrier protein, at a polysaccharide concentration of about 28 to 36, preferably about 32 μg/mL; wherein the EPA-4 carrier protein comprises the amino acid sequence of SEQ ID NO: 3, and wherein the O1A, O2, O4, O6A, O15, O16, O18A, O25B, and O75 antigen polysaccharides comprise the structures of Formulas (O1A), (O2), (O4-Glc+), (O6A), (O15), (O16), (O18A), (O25B), and (O75), respectively, as shown in Table 1, wherein each n is independently an integer of 1 to 100, preferably of 3 to 50, for example 5 to 40, more preferably of 5 to 30, for example 7 to 25, for example 10 to 20.

In certain embodiments, such a composition further comprises:
(x) a bioconjugate of an E. coli O75 antigen polysaccharide covalently linked to an EPA-4 carrier protein, at a polysaccharide concentration of about 12 to 20, preferably about 16 μg/mL;
wherein the O8 antigen polysaccharide comprises the structure of Formula (O8) as shown in Table 1, wherein n is an integer of 1 to 100, preferably of 3 to 50, for example 5 to 40, more preferably of 5 to 30, for example 7 to 25, for example 10 to 20.

In one embodiment, there is provided a composition comprising a bioconjugate of E. coli O75 antigen polysaccharide covalently linked to a carrier protein, wherein the O75 antigen polysaccharide is at a concentration of at least about 32 μg/mL.

In one embodiment, there is provided a composition comprising:
(i) a bioconjugate of an E. coli O1A antigen polysaccharide covalently linked to a carrier protein, wherein the E. coli O1A antigen polysaccharide comprises the structure of Formula (O1A);
(ii) a bioconjugate of an E. coli O2 antigen polysaccharide covalently linked to a carrier protein, wherein the E. coli O2 antigen polysaccharide comprises the structure of Formula (O2);
(iii) a bioconjugate of an E. coli glucosylated O4 antigen polysaccharide covalently linked to a carrier protein, wherein the E. coli glucosylated O4 antigen polysaccharide comprises the structure of Formula (O4-Glc+);
(iv) a bioconjugate of an E. coli O6A antigen polysaccharide covalently linked to a carrier protein, wherein the E. coli O6A antigen polysaccharide comprises the structure of Formula (O6A);
(v) a bioconjugate of an E. coli O15 antigen polysaccharide covalently linked to a carrier protein, wherein the E. coli O15 antigen polysaccharide comprises the structure of Formula (O15);
(vi) a bioconjugate of an E. coli O16 antigen polysaccharide covalently linked to a carrier protein, wherein the E. coli O16 antigen polysaccharide comprises the structure of Formula (O16);
(vii) a bioconjugate of an E. coli O18A antigen polysaccharide covalently linked to a carrier protein, wherein the E. coli O18A antigen polysaccharide comprises the structure of Formula (O18A);
(viii) a bioconjugate of an E. coli O25B antigen polysaccharide covalently linked to a carrier protein, wherein the E. coli O25B antigen polysaccharide comprises the structure of Formula (O25B); and
(ix) a bioconjugate of an E. coli O75 antigen polysaccharide covalently linked to a carrier protein, wherein the E. coli O75 antigen polysaccharide comprises the structure of Formula (O75);
wherein each of the structures of Formulas (O1A), (O2), (O4-Glc+), (O6A), (O15), (O16), (O18A), (O25B), and (O75) is shown in Table 1, wherein each n is independently an integer of 1 to 100, preferably of 3 to 50, for example 5 to 40, more preferably of 5 to 30, for example 7 to 25, for example 10 to 20,
wherein the E. coli antigen polysaccharides present in the composition consist of O1A, O2, O4-Glc+, O6A, O15, O16, O18A, O25B, and O75. Preferably, the carrier protein is detoxified Exotoxin A of P. aeruginosa (EPA-4), more preferably the EPA-4 comprises the amino acid sequence of SEQ ID NO: 3. Preferably, the ratio of concentrations of O75 antigen polysaccharide to O1A, O2, and/or O6A antigen polysaccharide is about 1.2:1 to about 8:1, preferably about 1.5:1 to about 4:1, more preferably about 2:1. Preferably the E. coli antigen polysaccharides present in the composition consist of O1A, O2, O4-Glc+, O6A, O15, O16, O18A, O25B, and O75 at a ratio of 1:1:1:1:1:1:1:2:2.

In one general aspect, provided herein is a composition comprising E. coli O75 and O6 antigen polysaccharides, wherein each of the antigen polysaccharides is independently covalently linked to a carrier protein, and wherein the ratio of concentrations of O75 antigen polysaccharide to O6 antigen polysaccharide is about 1.2:1 to about 8:1, preferably about 1.5:1 to about 4:1, preferably about 1.5:1 to about 2.5:1, more preferably about 2:1.

In certain embodiments, the composition further comprises one or more, preferably all, of E. coli O1, O2, O4, O15, O16, O18, O25 antigen polysaccharides, wherein each of the antigen polysaccharides is independently covalently linked to a carrier protein, preferably wherein the O1 antigen is O1A, the O4 is glucosylated, the O6 antigen is O6A, the O18 antigen is O18A, and the O25 antigen is O25B.

In certain embodiments,
the E. coli O1 antigen polysaccharide comprises the structure of Formula (O1A) shown in Table 1,
the E. coli O2 antigen polysaccharide comprises the structure of Formula (O2) shown in Table 1,
the E. coli O4 antigen polysaccharide comprises the structure of Formula (O4-Glc+) shown in Table 1,
the E. coli O6 antigen polysaccharide comprises the structure of Formula (O6A) shown in Table 1,
the E. coli O15 antigen polysaccharide comprises the structure of Formula (O15) shown in Table 1,
the E. coli O16 antigen polysaccharide comprises the structure of Formula (O16) shown in Table 1,
the E. coli O18 antigen polysaccharide comprises the structure of Formula (O18A) shown in Table 1,
the E. coli O25 antigen polysaccharide comprises the structure of Formula (O25B) shown in Table 1, and
the E. coli O75 antigen polysaccharide comprises the structure of Formula (O75) shown in Table 1,
wherein each n is independently an integer of 1 to 100, preferably of 3 to 50, for example 5 to 40, preferably of 5 to 30, for example 7 to 25, for example 10 to 20.

In certain embodiments, the weight ratio of the E. coli antigen polysaccharides of O1:O2:O4:O6:O15:O16:O18:O25:O75 is 1:1:1:1:1:1:1:2:2.

In certain embodiments, the concentration of the O75 antigen polysaccharide is from about 8 to about 50 μg/mL, preferably 12 to 40 μg/mL, e.g. 16-32 μg/mL, preferably about 32 μg/mL.

In certain embodiments, the composition further comprises at least one additional E. coli antigen polysaccharide covalently linked to a carrier protein.

In a particular embodiment, the at least one additional E. coli antigen polysaccharide comprises O8 antigen polysaccharide with Formula (O8) shown in Table 1, wherein n is an integer of 1 to 100, preferably of 3 to 50, for example 5 to 40, preferably of 5 to 30, for example 7 to 25, for example 10 to 20.

In certain embodiments, each carrier protein is independently selected from the group consisting of detoxified Exotoxin A of P. aeruginosa (EPA), E. coli flagellin (FliC), CRM197, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

In a particular embodiment, the carrier protein is detoxified exotoxin A of Pseudomonas aeruginosa (EPA) or CRM197.

In certain embodiments, the carrier protein comprises 1 to 20, such as 1 to 10, or 2 to 4, glycosylation consensus sequences having the amino acid sequence of SEQ ID NO: 1, preferably the consensus sequences having the amino acid sequence of SEQ ID NO: 2, most preferably the carrier protein comprises four of the glycosylation consensus sequences.

In a particular embodiment, each carrier protein is EPA comprising the amino acid sequence of SEQ ID NO: 3.

In certain embodiments, the E. coli antigen polysaccharides are covalently linked to the carrier protein by bioconjugation or by chemical conjugation. Chemical conjugation can, for example, include reductive amination chemistry (RAC), single-end conjugation, conjugation with a (2-((2-oxoethyl)thio)ethyl) carbamate (eTEC) spacer, cyanylation chemistry (CNBr, CDAP) with or without ADH spacer, thioether chemistry (maleimide/bromoacetyl linker based), or EDC-N-Hydroxy succinimide zero linker chemistry.

In certain embodiments, the E. coli antigen polysaccharides are covalently linked to the carrier protein by bioconjugation, preferably the polysaccharide is covalently linked to an Asn residue in a glycosylation site in the carrier protein.

In another aspect, provided herein is a composition comprising a bioconjugate of an E. coli O1A antigen polysaccharide covalently linked to a detoxified Exotoxin A of P. aeruginosa (EPA-4) carrier protein, wherein the E. coli O1A antigen polysaccharide comprises the structure of Formula (O1A);
a bioconjugate of an E. coli O2 antigen polysaccharide covalently linked to an EPA-4 carrier protein, wherein the E. coli O2 antigen polysaccharide comprises the structure of Formula (O2);
a bioconjugate of an E. coli glucosylated O4 antigen polysaccharide covalently linked to an EPA-4 carrier protein, wherein the E. coli glucosylated O4 antigen polysaccharide comprises the structure of Formula (O4-Glc+);
a bioconjugate of an E. coli O6A antigen polysaccharide covalently linked to an EPA-4 carrier protein, wherein the E. coli O6A antigen polysaccharide comprises the structure of Formula (O6A);
a bioconjugate of an E. coli O15 antigen polysaccharide covalently linked to an EPA-4 carrier protein, wherein the E. coli O15 antigen polysaccharide comprises the structure of Formula (O15);
a bioconjugate of an E. coli O16 antigen polysaccharide covalently linked to an EPA-4 carrier protein, wherein the E. coli O16 antigen polysaccharide comprises the structure of Formula (O16);
a bioconjugate of an E. coli O18A antigen polysaccharide covalently linked to an EPA-4 carrier protein, wherein the E. coli O18A antigen polysaccharide comprises the structure of Formula (O18A);
a bioconjugate of an E. coli O25B antigen polysaccharide covalently linked to an EPA-4 carrier protein, wherein the E. coli O25B antigen polysaccharide comprises the structure of Formula (O25B); and
a bioconjugate of an E. coli O75 antigen polysaccharide covalently linked to an EPA-4 carrier protein, wherein the E. coli O75 antigen polysaccharide comprises the structure of Formula (O75);
wherein the EPA-4 comprises the amino acid sequence of SEQ ID NO: 3,
wherein each of the structures of Formulas (O1A), (O2), (O4-Glc+), (O6A), (O15), (O16), (O18A), (O25B), and (O75) is shown in Table 1, wherein each n is independently an integer of 1 to 100, preferably of 3 to 50, for example 5 to 40, more preferably of 5 to 30, for example 7 to 25, for example 10 to 20, and
wherein the ratio of O75 antigen polysaccharide to O6A antigen polysaccharide is about 1.2:1 to about 8:1, preferably about 1.5:1 to about 4:1, more preferably about 2:1.

In certain embodiments, the composition further comprises from 1 to 15 additional E. coli antigen polysaccharides each independently covalently linked to a carrier protein.

In certain embodiments, the composition comprises a bioconjugate of an E. coli glucosylated O4 antigen polysaccharide covalently linked to a carrier protein, wherein the E. coli glucosylated O4 antigen polysaccharide comprises the structure of Formula (O4-Glc+) shown in Table 1, wherein n is an integer of 3 to 50, for example 5 to 40, preferably of 5 to 30, for example 7 to 25, for example 10 to 20, wherein the bioconjugate of an E. coli glucosylated O4 antigen polysaccharide covalently linked to a carrier protein has been produced in an E. coli cell that comprises:
(i) a nucleotide sequence of an rfb gene cluster for the E. coli O4 antigen polysaccharide;
(ii) a nucleotide sequence encoding a glucosyl transferase having at least 80%, preferably at least 90%, preferably at least 95% sequence identity to SEQ ID NO: 4, wherein the glucosyl transferase is capable of modifying the E. coli O4 antigen polysaccharide to produce the E. coli glucosylated O4 antigen polysaccharide;
(iii) nucleotide sequences encoding a translocase and a glycosyltransferase having at least 80%, preferably at least 90%, preferably at least 95% sequence identity to SEQ ID NOs: 7 and 8 respectively, wherein the translocase is capable of translocating bactoprenol-linked glucose and the glycosyltransferase is capable of glucosylating bactoprenol;
(iv) a nucleotide sequence encoding the carrier protein; and
(v) a nucleotide sequence encoding an oligosaccharyl transferase capable of covalently linking the E. coli glucosylated O4 antigen polysaccharide to the carrier protein, preferably wherein the oligosaccharyl transferase is PglB from Campylobacter jejuni.

In certain embodiments, the compositions described herein additionally comprise a preservative, such as the mercury derivative thimerosal. In a specific embodiment, the pharmaceutical compositions described herein comprise 0.001% to 0.01% thimerosal. In other embodiments, the pharmaceutical compositions described herein do not comprise a preservative.

In certain embodiments, the compositions described herein (e.g., the immunogenic compositions) comprise, or are administered in combination with, an adjuvant. The adjuvant for administration in combination with a composition described herein may be administered before (e.g. within 72 hours, 48 hours, 24 hours, 12 hours, 6 hours, 2 hours, 1 hour, 10 minutes), concomitantly with, or after (e.g. within 72 hours, 48 hours, 24 hours, 12 hours, 6 hours, 2 hours, 1 hour, 10 minutes) administration of said composition. In some embodiments, the term “adjuvant” refers to a compound that when administered in conjunction with or as part of a composition described herein augments, enhances and/or boosts the immune response to a bioconjugate, but when the adjuvant compound is administered alone does not generate an immune response to the bioconjugate. In some embodiments, the adjuvant generates an immune response to the bioconjugate peptide and does not produce an allergy or other adverse reaction. Adjuvants can enhance an immune response by several mechanisms including, e.g., lymphocyte recruitment, stimulation of B and/or T cells, and stimulation of macrophages. In certain preferred embodiments, the compositions described herein do not comprise an adjuvant besides the bioconjugates, and/or are not administered in combination with an adjuvant besides the bioconjugates (in case the bioconjugates would comprise some intrinsic adjuvant properties, these would be disregarded and no extrinsic adjuvant would be added in these embodiments).

Examples of suitable adjuvants include, but are not limited to, aluminum salts (alum) (such as aluminum hydroxide, aluminum phosphate, aluminum sulfate and aluminum oxide, including nanoparticles comprising alum or nanoalum formulations), calcium phosphate, monophosphoryl lipid A (MPL) or 3-de-O-acylated monophosphoryl lipid A (3D-MPL) (see e.g., GB2220211, EP0971739, EP 1194166, U.S. Pat. No. 6,491,919), AS01, AS02, AS03 and AS04 (all GlaxoSmithKline; see e.g. EP1126876, U.S. Pat. No. 7,357,936 for AS04, EP0671948, EP0761231, U.S. Pat. No. 5,750,110 for AS02), MF59 (Novartis), imidazopyridine compounds (see WO2007/109812), imidazoquinoxaline compounds (see WO2007/109813), delta-inulin, STING-activating synthetic cyclic-di-nucleotides (e.g. US20150056224), combinations of lecithin and carbomer homopolymers (e.g. U.S. Pat. No. 6,676,958), and saponins, such as QuilA and QS21 (see e.g. Zhu D and W Tuo, 2016, Nat Prod Chem Res 3: e113, Matrix M, Iscoms, Iscomatrix, etc, optionally in combination with QS7 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG. Further examples of adjuvants are liposomes containing immune stimulants such as MPL and QS21 such as in ASOlE and AS01B (e.g. US 2011/0206758). Other examples of adjuvants are imidazoquinolines (such as imiquimod and R848). See, e.g., Reed G, et al., 2013, Nature Med, 19: 1597-1608. In certain embodiments, the adjuvant contains a toll-like receptor 4 (TLR4) agonist. TLR4 agonists are well known in the art, see e.g. Ireton G C and SG Reed, 2013, Expert Rev Vaccines 12: 793-807. In certain embodiments, the adjuvant comprises a TLR4 agonist comprising lipid A, or an analog or derivative thereof, such as MPL, 3D-MPL, RC529 (e.g. EP1385541), PET-lipid A, GLA (glycopyranosyl lipid adjuvant, a synthetic disaccharide glycolipid; e.g. US20100310602, U.S. Pat. No. 8,722,064), SLA (e.g. Carter D et al, 2016, Clin Transl Immunology 5: e108 (doi: 10.1038/cti.2016.63), which describes a structure-function approach to optimize TLR4 ligands for human vaccines), PHAD (phosphorylated hexaacyl disaccharide), 3D-PHAD (the structure of which is the same as that of GLA), 3D-(6-acyl)-PHAD (3D(6A)-PHAD) (PHAD, 3D-PHAD, and 3D(6A)PHAD are synthetic lipid A variants, see e.g. avantilipids.com/divisions/adjuvants, which also provide structures of these molecules), E6020 (CAS Number 287180-63-6), ONO4007, OM-174, and the like.

In certain embodiments, the compositions described herein do not comprise, and are not administered in combination with, an adjuvant.

In certain embodiments, the compositions described herein are formulated to be suitable for the intended route of administration to a subject. For example, the compositions described herein may be formulated to be suitable for subcutaneous, parenteral, oral, intradermal, transdermal, colorectal, intraperitoneal, and rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration. In certain embodiments, the compositions described herein are administered by intramuscular injection.

In certain embodiments, the compositions described herein additionally comprise one or more buffers, e.g., Tris-buffered saline, phosphate buffer, or sucrose phosphate glutamate buffer.

In certain embodiments, the compositions described herein additionally comprise one or more salts, e.g., Tris-hydrochloride, sodium chloride, calcium chloride, potassium chloride, sodium phosphate, monosodium glutamate, or aluminum salts (e.g., aluminum hydroxide, aluminum phosphate, alum (potassium aluminum sulfate), or a mixture of such aluminum salts).

The compositions described herein can be included in a container, pack, or dispenser together with instructions for administration.

The compositions described herein can be stored before use, e.g., the compositions can be stored frozen (e.g., at about −20° C. or at about −70° C.); stored in refrigerated conditions (e.g., at about 4° C.); or stored at room temperature.

In one aspect the invention also provides methods to prepare compositions according to the invention, comprising providing each of the required O-antigen conjugates (e.g., by obtaining or manufacturing these, e.g., in the form of drug substances), and mixing them in the desired ratios and/or amounts to obtain a composition of the invention (e.g., a multivalent E. coli, particularly ExPEC, vaccine composition, sometimes referred to as drug product).

### Methods of Inducing an Immunological Response

Bioconjugates and compositions provided herein can be used to induce antibodies against an E. coli O-antigen in a subject, or to vaccinate a subject against E. coli. The methods of inducing an immune response in a subject described herein result in vaccination of the subject against infection by the ExPEC strains whose O-antigens are present in the composition(s). When an O-antigen subtype is used, a method of the invention can also induce immune response to another O-antigen subtype having similar antigenicity. Examples are cross-reactivity between subserotypes, e.g. immunization with O25B antigen induces antibodies that recognize O-LPS of both O25B and O25A serotypes, and immunization with glucosylated O4 induces antibodies that recognize O-LPS of both glucosylated O4 and non-glucosylated O4 serotypes, and in other examples there can also be cross-immunization towards other serotypes that have different O-antigens but still share some similarity in the antigenic structures (e.g., some sera appear to cross-react in serotyping studies).

In some embodiments, the subject is human. In some embodiments, the subject is a human having or at risk of having an ExPEC infection or an invasive ExPEC disease.

In some embodiments, the invasive ExPEC disease comprises sepsis. In some embodiments, the invasive ExPEC disease comprises bacteremia. In some embodiments, the invasive ExPEC disease comprises one or more of urinary tract infection, a surgical-site infection, an abdominal or pelvic infection, pneumonia, osteomyelitis, cellulitis, sepsis, bacteremia, a wound infection, pyelonephritis, meningitis, peritonitis, cholangitis, soft-tissue infections, pyomyositis, septic arthritis, endophthalmitis, suppurative thyroiditis, sinusitis, endocarditis, and prostatitis.

In certain embodiments, the immune response induced in a subject following administration of a composition described herein limits the severity of or prevents an invasive ExPEC disease in the subject. In one embodiment, the subject has an E. coli (e.g., ExPEC) infection at the time of administration. In a preferred embodiment, the subject does not have an E. coli (e.g., ExPEC) infection at the time of administration.

In certain embodiments, the immune response induced in a subject following administration of a composition described herein is effective to prevent or reduce a symptom resulting from an ExPEC infection, preferably in at least 30%, more preferably at least 40%, such as at least 50%, of the subjects administered with the composition. Symptoms of ExPEC infection may vary depending on the nature of the infection and may include, but are not limited to: dysuria, increased urinary frequency or urgency, pyuria, hematuria, back pain, pelvic pain, pain while urinating, fever, chills, and/or nausea (e.g., in subjects having a urinary tract infection caused by ExPEC); high fever, headache, stiff neck, nausea, vomiting, seizures, sleepiness, and/or light sensitivity (e.g., in subjects having meningitis caused by ExPEC); fever, increased heart rate, increased respiratory rate, decreased urine output, decreased platelet count, abdominal pain, difficulty breathing, and/or abnormal heart function (e.g., in subjects having sepsis caused by ExPEC).

In certain embodiments, the immune response induced in a subject following administration of a composition described herein is effective to reduce the likelihood of hospitalization of a subject suffering from an ExPEC infection. In some embodiments, the immune response induced in a subject following administration of a composition described herein is effective to reduce the duration of hospitalization of a subject suffering from an ExPEC infection.

In certain embodiments, vaccination with a composition of the invention is for preventing an invasive ExPEC disease (IED), e.g., urosepsis, bacteremia, sepsis, etc. In certain embodiments, vaccination is to prevent or reduce the occurrence or severity of urinary tract infections. In certain embodiments, an IED can be hospital-acquired, e.g. in patients undergoing urogenital and/or abdominal procedures or surgeries. In certain embodiments, an IED can be healthcare-associated, e.g. in patients receiving health care for another condition, for instance via central lines, catheters, etc, e.g. in a hospital, ambulatory surgical center, end-stage renal disease facility, long-term care facility, etc. In certain embodiments, the IED can be community-acquired, e.g. in a patient that was not recently exposed to healthcare risks.

In some embodiments, a method of inducing an immune response to extra-intestinal pathogenic E. coli (ExPEC) in a subject comprises administering to the subject a composition described herein.

In some embodiments, a method of inducing an immune response to extra-intestinal pathogenic E. coli (ExPEC) in a subject comprises administering to the subject a first effective amount of E. coli O75 antigen polysaccharide, and a second effective amount of E. coli O6 antigen polysaccharide, wherein each of the antigen polysaccharides is independently covalently linked to a carrier protein, and wherein the ratio of the first effective amount to the second effective amount is about 1.2:1 to about 8:1, preferably about 1.5:1 to about 4:1, e.g. about 1.5:1, 1.6:1, 1.7:1, 1.8:1, 1.9:1, 2.0:1, 2.1:1, 2.2:1, 2.3:1, 2.4:1, 2.5:1, more preferably about 2:1. In some preferred embodiments, the O75 antigen polysaccharide covalently linked to a carrier protein and the O6 antigen polysaccharide covalently linked to a carrier protein are administered as one composition. In some alternative embodiments, the O75 antigen polysaccharide covalently linked to a carrier protein and the O6 antigen polysaccharide covalently linked to a carrier protein are administered as a combination of separate compositions.

In some embodiments, a method of inducing an immune response to extra-intestinal pathogenic E. coli (ExPEC) in a subject further comprises administering to the subject one or more, preferably all, of E. coli O1, O2, O4, O15, O16, O18, O25 antigen polysaccharides, wherein each of the antigen polysaccharides is independently covalently linked to a carrier protein. Preferably the O1 antigen is O1A, the O4 antigen is glucosylated O4 antigen polysaccharide, the O6 antigen is O6A, the O18 antigen is O18A, and the O25 antigen is O25B. Preferably the O1-, O2-, O4-, O6-, O15-, O16-, O18-, O25-, and O75-antigens respectively have the structures of Formulas (O1A), (O2), (O4-Glc+), (O6A), (O15), (O16), (O18A), (O25B), and (O75) as shown in Table 1, wherein each n is independently an integer of 1 to 100, preferably of 3 to 50, for example 5 to 40, preferably of 5 to 30, for example 7 to 25, for example 10 to 20. According to the invention, the ratio of O75 antigen polysaccharide to O6 antigen polysaccharide is about 1.2:1 to about 8:1, preferably about 1.5:1 to about 4:1, more preferably about 2:1. In some embodiments, the method further comprises administering to the subject from 1 to 15 additional E. coli antigen polysaccharides, each independently covalently linked to a carrier protein. In certain embodiments such additional E. coli antigens comprise O8 antigen polysaccharide, preferably having the structure of Formula (O8) as shown in Table 1, wherein n is an integer of 1 to 100, preferably of 3 to 50, for example 5 to 40, preferably of 5 to 30, for example 7 to 25, for example 10 to 20. Preferably, each E. coli antigen polysaccharide covalently linked to a carrier protein is a bioconjugate. In some embodiments, the bioconjugates are administered as one composition. In some embodiments, the bioconjugates are administered as a combination of two or more separate compositions. It is preferred to limit the number of separate administrations, so use of single compositions comprising most or all of the antigens is preferred.

In one embodiment is a method of administering a composition for inducing an immune response to E. coli, preferably extra-intestinal pathogenic E. coli (ExPEC), wherein the composition comprises a bioconjugate of E. coli O75 antigen polysaccharide covalently linked to a carrier protein, and wherein the concentration of O75 antigen polysaccharide administered is at least 16 μg per dose.

In some embodiments, a method of inducing an immune response to extra-intestinal pathogenic E. coli (ExPEC) in a subject comprises administering to the subject a first effective amount of E. coli O75 antigen polysaccharide, and a second effective amount of E. coli O1 antigen polysaccharide, wherein each of the antigen polysaccharides is independently covalently linked to a carrier protein, and wherein the ratio of the first effective amount to the second effective amount is between about 1.2:1 to about 8:1, preferably between about 1.5:1 to about 4:1, e.g. about 1.5:1, 1.6:1, 1.7:1, 1.8:1, 1.9:1, 2.0:1, 2.1:1, 2.2:1, 2.3:1, 2.4:1, 2.5:1, more preferably about 2:1. In some preferred embodiments, the O75 antigen polysaccharide covalently linked to a carrier protein and the O1 antigen polysaccharide covalently linked to a carrier protein are administered as one composition. In some alternative embodiments, the O75 antigen polysaccharide covalently linked to a carrier protein and the O1 antigen polysaccharide covalently linked to a carrier protein are administered as a combination of compositions. In some embodiments, the method further comprises administering to the subject one or more, preferably all, of E. coli O2, O4, O6, O15, O16, O18, O25 antigen polysaccharides, wherein each of the antigen polysaccharides is independently covalently linked to a carrier protein. Preferably the O1 antigen is O1A, the O4 antigen is glucosylated O4 antigen polysaccharide, the O6 antigen is O6A, the O18 antigen is O18A, and the O25 antigen is O25B. Preferably the O1-, O2-, O4-, O6-, O15-, O16-, O18-, O25-, and O75-antigens respectively have the structures of Formulas (O1A), (O2), (O4-Glc+), (O6A), (O15), (O16), (O18A), (O25B), and (O75) as shown in Table 1, wherein each n is independently an integer of 1 to 100, preferably of 3 to 50, for example 5 to 40, preferably of 5 to 30, for example 7 to 25, for example 10 to 20. According to the invention, the ratio of O75 antigen polysaccharide to O1 antigen polysaccharide is about 1.2:1 to about 8:1, preferably about 1.5:1 to about 4:1, more preferably about 2:1. In some embodiments, the method further comprises administering to the subject from 1 to 15 additional E. coli antigen polysaccharides, each independently covalently linked to a carrier protein. In certain embodiments such additional E. coli antigens comprise O8 antigen polysaccharide, preferably having the structure of Formula (O8) as shown in Table 1, wherein n is an integer of 1 to 100, preferably of 3 to 50, for example 5 to 40, preferably of 5 to 30, for example 7 to 25, for example 10 to 20. Preferably, each E. coli antigen polysaccharide covalently linked to a carrier protein is a bioconjugate. In some embodiments, the bioconjugates are administered as one composition. In some embodiments, the bioconjugates are administered as a combination of two or more separate compositions. It is preferred to limit the number of separate administrations, so use of single compositions comprising most or all of the antigens is preferred.

In some embodiments, a method of inducing an immune response to extra-intestinal pathogenic E. coli (ExPEC) in a subject comprises administering to the subject a first effective amount of E. coli O75 antigen polysaccharide, and a second effective amount of E. coli O2 antigen polysaccharide, wherein each of the antigen polysaccharides is independently covalently linked to a carrier protein, and wherein the ratio of the first effective amount to the second effective amount is about 1.2:1 to about 8:1, preferably about 1.5:1 to about 4:1, e.g. about 1.5:1, 1.6:1, 1.7:1, 1.8:1, 1.9:1, 2.0:1, 2.1:1, 2.2:1, 2.3:1, 2.4:1, 2.5:1, more preferably about 2:1. In some preferred embodiments, the O75 antigen polysaccharide covalently linked to a carrier protein and the O2 antigen polysaccharide covalently linked to a carrier protein are administered as one composition. In some alternative embodiments, the O75 antigen polysaccharide covalently linked to a carrier protein and the O2 antigen polysaccharide covalently linked to a carrier protein are administered as a combination of compositions. In some embodiments, the method further comprises administering to the subject one or more, preferably all, of E. coli O1, O4, O6, O15, O16, O18, O25 antigen polysaccharides, wherein each of the antigen polysaccharides is independently covalently linked to a carrier protein. Preferably the O1 antigen is O1A, the O4 antigen is glucosylated O4 antigen polysaccharide, the O6 antigen is O6A, the O18 antigen is O18A, and the O25 antigen is O25B. Preferably the O1-, O2-, O4-, O6-, O15-, O16-, O18-, O25-, and O75-antigens respectively have the structures of Formulas (O1A), (O2), (O4-Glc+), (O6A), (O15), (O16), (O18A), (O25B), and (O75) as shown in Table 1, wherein each n is independently an integer of 1 to 100, preferably of 3 to 50, for example 5 to 40, preferably of 5 to 30, for example 7 to 25, for example 10 to 20. According to the invention, the ratio of O75 antigen polysaccharide to O2 antigen polysaccharide is about 1.2:1 to about 8:1, preferably about 1.5:1 to about 4:1, more preferably about 2:1. In some embodiments, the method further comprises administering to the subject from 1 to 15 additional E. coli antigen polysaccharides, each independently covalently linked to a carrier protein. Preferably, each E. coli antigen polysaccharide covalently linked to a carrier protein is a bioconjugate. In some embodiments, the bioconjugates are administered as one composition. In some embodiments, the bioconjugates are administered as a combination of two or more separate compositions. It is preferred to limit the number of separate administrations, so use of single compositions comprising most or all of the antigens are preferred.

In some embodiments, a method of inducing an immune response to extra-intestinal pathogenic E. coli (ExPEC) in a subject comprises administering to the subject a first effective amount of E. coli O75 antigen polysaccharide, and a second effective amount of E. coli O25 antigen polysaccharide, wherein each of the antigen polysaccharides is independently covalently linked to a carrier protein, and wherein the ratio of the first effective amount to the second effective amount is about 1:4 and 1:0.5, preferably between about 1:2 and 1:1, more preferably about 1:1, e.g. about 1:1.5, 1:1.4, 1:1.3, 1:1.2, 1:1.1, 1:1.0, 1:0.9, 1:0.8, 1:0.7, 1:0.6, or 1:0.5. In some preferred embodiments, the O75 antigen polysaccharide covalently linked to a carrier protein and the O25 antigen polysaccharide covalently linked to a carrier protein are administered as one composition. In some alternative embodiments, the O75 antigen polysaccharide covalently linked to a carrier protein and the O25 antigen polysaccharide covalently linked to a carrier protein are administered as a combination of compositions. In some embodiments, the method further comprises administering to the subject one or more, preferably all, of E. coli O1, O2, O4, O6, O15, O16, O18 antigen polysaccharides, wherein each of the antigen polysaccharides is independently covalently linked to a carrier protein. Preferably the O1 antigen is O1A, the O4 antigen is glucosylated O4 antigen polysaccharide, the O6 antigen is O6A, the O18 antigen is O18A, and the O25 antigen is O25B. Preferably the O1-, O2-, O4-, O6-, O15-, O16-, O18-, O25-, and O75-antigens respectively have the structures of Formulas (O1A), (O2), (O4-Glc+), (O6A), (O15), (O16), (O18A), (O25B), and (O75) as shown in Table 1, wherein each n is independently an integer of 1 to 100, preferably of 3 to 50, for example 5 to 40, preferably of 5 to 30, for example 7 to 25, for example 10 to 20. According to the invention, the ratio of O75 antigen polysaccharide to O25 antigen polysaccharide is about 1:4 to about 1:0.5, preferably between about 1:2 and 1:1, more preferably about 1:1. In some embodiments, the method further comprises administering to the subject from 1 to 15 additional E. coli antigen polysaccharides, each independently covalently linked to a carrier protein. In certain embodiments such additional E. coli antigens comprise O8 antigen polysaccharide, preferably having the structure of Formula (O8) as shown in Table 1, wherein n is an integer of 1 to 100, preferably of 3 to 50, for example 5 to 40, preferably of 5 to 30, for example 7 to 25, for example 10 to 20. Preferably, each E. coli antigen polysaccharide covalently linked to a carrier protein is a bioconjugate. In some embodiments, the bioconjugates are administered as one composition. In some embodiments, the bioconjugates are administered as a combination of two or more separate compositions. It is preferred to limit the number of separate administrations, so use of single compositions comprising most or all of the antigens are preferred.

In certain embodiments, the compositions, combinations, and bioconjugates described herein can be administered to a subject to induce an immune response that includes the production of antibodies, preferably antibodies having opsonophagocytic activity. Such antibodies can be isolated using techniques known to one of skill in the art (e.g., immunoaffinity chromatography, centrifugation, precipitation, etc.).

The ability of the bioconjugates and compositions described herein to generate an immune response in a subject can be assessed using any approach known to those of skill in the art or described herein. In some embodiments, the ability of a bioconjugate to generate an immune response in a subject can be assessed by immunizing a subject (e.g., a mouse, rat, rabbit, or monkey) or set of subjects with a bioconjugate described herein and immunizing an additional subject (e.g., a mouse, rat, rabbit, or monkey) or set of subjects with a control (e.g., PBS). The subjects or set of subjects can subsequently be challenged with ExPEC and the ability of the ExPEC to cause disease (e.g., UTI, bacteremia, or other disease) in the subjects or set of subjects can be determined. Those skilled in the art will recognize that if the subject or set of subjects immunized with the control suffer(s) from disease subsequent to challenge with the ExPEC but the subject or set of subjects immunized with a bioconjugate(s) or composition thereof described herein suffer less from or do not suffer from disease, then the bioconjugate is able to generate an immune response in a subject. The ability of a bioconjugate(s) or composition thereof described herein to induce antiserum that cross-reacts with an O antigen from ExPEC can be tested by, e.g., an immunoassay, such as an ELISA (see e.g., Van den Dobbelsteen et al, 2016, Vaccine 34: 4152-4160), or an ECL-based immunoassay.

For example, the ability of the bioconjugates described herein to generate an immune response in a subject can be assessed using a serum bactericidal assay (SBA) or opsonophagocytic killing assay (OPK assay, or OPKA), which represents an established and accepted method that has been used to obtain approval of glycoconjugate-based vaccines. Such assays are well-known in the art and, briefly, comprise the steps of generating and isolating antibodies against a target of interest (e.g., an O antigen polysaccharide, e.g., E. coli O75 antigen polysaccharide) by administering to a subject (e.g., a mouse, rat, rabbit, or monkey) a compound that elicits such antibodies. Subsequently, the bactericidal capacity of the antibodies can be assessed by, e.g., culturing the bacteria in question (e.g., E. coli of the relevant serotype) in the presence of the antibodies and complement and—depending on the assay—neutrophilic cells and assaying the ability of the antibodies to mediate killing and/or neutralization of the bacteria, e.g., using standard microbiological approaches. For an example of OPK assay for E. coli bioconjugate vaccines, see e.g. Abbanat et al, 2017, Clin. Vaccine Immunol. 24: e00123-17. An OPK assay can be performed in monoplex or multiplex format, of which multiplex format (e.g. testing multiple serotypes at the same time) is typically preferred. A multiplex OPK assay is sometimes referred to herein as “MOPA”.

In particular embodiments, wherein a composition provided herein comprises a bioconjugate of an E. coli O75 antigen polysaccharide and at least a bioconjugate of an E. coli O6A antigen polysaccharide, an effective amount of the E. coli O75 antigen polysaccharide is about 1.2 to 8 times, e.g. about 2 to 4 times higher, such as 1.5, 2, 3, 4, 5, 6, 7 or 8 times higher than the concentration of any of the other bioconjugates present in the composition. In such embodiments, an effective amount of the E. coli O75 antigen polysaccharide is for instance about 5 to 18 μg per administration, such as 5, 6, 7, 8, 9, 10, 11, 12, 13, 14, 15, 16, 17, 18 μg per administration. Preferably, about 8-16 μg of the O75 antigen polysaccharide is administered per administration.

In certain embodiments, a composition described herein is administered to a subject in combination with one or more other therapies (e.g., antibacterial or immunomodulatory therapies). The one or more other therapies can be beneficial in the treatment or prevention of an ExPEC infection or can ameliorate a symptom or condition associated with an ExPEC infection. In some embodiments, the one or more other therapies are pain relievers or anti-fever medications. In certain embodiments, the therapies are administered less than 5 minutes apart to less than 1 week apart. Any anti-bacterial agents known to one of skill in the art (e.g. antibiotics) may be used in combination with a composition described herein.

In certain embodiments, a bioconjugate or composition according to the invention is administered to a subject once. In certain embodiments, a bioconjugate or composition according to the invention is administered to a subject more than once, e.g. in a prime-boost regimen. In certain embodiments, the time between two administrations is at least two weeks, at least one month, at least two months, at least three months, at least six months, at least one year, at least two years, at least five years, at least ten years, or at least fifteen years. In humans, a desired immune response can typically be generated by a single administration of a bioconjugate or composition according to the invention. In certain embodiments, a repeat administration after, for instance ten years, is provided.

The compositions provided herein can be used to induce antibodies against E. coli 0 antigens in a subject, and to vaccinate a subject against E. coli, in particular extra-intestinal pathogenic E. coli (ExPEC). As used herein, “subject” means any animal, preferably a mammal, to whom will be or has been administered a bioconjugate or composition provided herein. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc. In certain embodiments, a subject is a human. A human subject may be of any age. In certain embodiments, a subject is a human of about two months to about 18 years old, e.g. of 1 year to 18 years old. In certain embodiments, a subject is a human of at least 18 years old. In certain embodiments, a subject is a human of 15 to 50 years old, e.g. 18 to 45 years old, e.g. 20 to 40 years old. In certain embodiments, a subject is a human male. In certain embodiments, a subject is a human female. In certain embodiments, a subject is immunocompromised. In certain embodiments, a subject is a human of at least 50 years, at least 55 years, at least 60 years, at least 65 years old. In certain embodiments, a subject is a human that is not older than 100 years, not older than 95 years, not older than 90 years, not older than 85 years, not older than 80 years, or not older than 75 years. In certain embodiments, a subject is a human of at least 60 years old, and not older than 85 years old. In certain embodiments, a subject is a human in stable health. In certain embodiments, a subject is a human adult of at least 60 and not more than 85 years old in stable health. In certain embodiments, a subject is a human that has a history of a urinary tract infection (UTI, i.e. a bacterial infection in the urethra, bladder, ureters, and/or kidneys; in some embodiments this includes pyelonephritis), i.e. having had at least one UTI episode in his or her life. In certain embodiments, a subject is a human that has a history of UTI in the past twenty, fifteen, twelve, ten, nine, eight, seven, six, five, four, three, two or one years. In certain embodiments, a subject is a human that has a history of UTI in the past two years. In certain embodiments, a subject is a human subject that has a history of recurrent UTI, i.e. having had at least two UTIs in six months or at least three UTIs in one year. In certain embodiments, a subject is a human subject that has a history of recurrent UTI in the past two years. In certain embodiments, a subject is a human of 60 years or older in stable health. In certain embodiments, a subject is a human of 60 years or older that has a history of UTI in the past two years. In certain embodiments, a subject is a human of at least 60 years and less than 75 years old that has a history of UTI in the past two years. In certain embodiments, a subject is a human subject of 75 years or older that has a history of UTI in the past two years. In certain embodiments, a subject is a patient scheduled for undergoing elective urogenital and/or abdominal procedures or surgeries, e.g. transrectal ultrasound-guided prostate needle biopsy (TRUS-PNB). In certain embodiments, a subject is a human that has a history of prostatitis, including but not limited to an acute bacterial prostatitis (ABP), i.e. a bacterial infection of the prostate, i.e. having had at least one prostatitis episode in his life, e.g. in the last ten, nine, eight, seven, six, five, four, three, two or one years.

In preferred embodiments, the immune response induced by the compositions or methods of inducing an immune response according to the invention includes antibodies that have opsonophagocytic activity. It has been shown that compositions comprising E. coli O antigen polysaccharides covalently linked to a carrier protein (i.e. glycoconjugates of E. coli O-antigens) can induce this type of functional antibodies in humans, and it has been shown that such antibodies mediate bacterial killing in vivo, and via this mechanism can protect against E. coli infections.

In another aspect, provided herein is a method of inducing an immune response to E. coli, preferably extra-intestinal pathogenic E. coli (ExPEC), in a subject, comprising administering to the subject a composition as described herein.

In another aspect, provided herein is a method of vaccinating a subject against E. coli, preferably extra-intestinal pathogenic E. coli (ExPEC), comprising administering to the subject a composition as described herein. In certain aspects, provided herein is a composition as described herein, for use in inducing antibodies against E. coli, preferably ExPEC. In certain aspects, provided herein is a composition as described herein, for use in vaccination against E. coli, preferably ExPEC. In certain aspects, provided herein is the use of a composition as described herein, for the manufacture of a medicament for inducing antibodies in a subject against E. coli, preferably ExPEC. In certain aspects, provided herein is the use of a composition as described herein, for the manufacture of a medicament for vaccinating a subject against E. coli, preferably ExPEC.

In certain aspects, provided herein is a composition as described herein for use in a method of inducing an immune response to E. coli, preferably extra-intestinal pathogenic E. coli (ExPEC), in a subject. In certain aspects, provided herein is use of a composition as described herein in the manufacture of a medicament for inducing an immune response to E. coli, preferably extra-intestinal pathogenic E. coli (ExPEC), in a subject.

In another aspect, provided herein is a method of inducing an immune response to E. coli, preferably extra-intestinal pathogenic E. coli (ExPEC), in a subject, comprising administering to the subject a first effective amount of E. coli O75 antigen polysaccharide, and a second effective amount of E. coli O6 antigen polysaccharide, wherein each of the antigen polysaccharides is independently covalently linked to a carrier protein, and wherein the ratio of the first effective amount to the second effective amount is about 1.2:1 to about 8:1, preferably about 1.5:1 to about 4:1, more preferably about 2:1.

In certain embodiments, the immune response limits the severity of or prevents an invasive ExPEC disease in the subject.

In certain embodiments, the invasive ExPEC disease comprises sepsis and/or bacteremia.

In certain embodiments, the method further comprises administering to the subject one or more, preferably all, of E. coli O1, O2, O4, O15, O16, O18, O25 antigen polysaccharides, wherein each of the antigen polysaccharides is independently covalently linked to a carrier protein,
preferably the O1 antigen is O1A, the O4 is glucosylated, the O6 antigen is O6A, the O18 antigen is O18A, and the O25 antigen is O25B,
more preferably wherein each of the O1A, O2, glucosylated O4, O6A, O15, O16, O18A, O25B, and O75 antigen polysaccharides comprise the structures of Formulas (O1A), (O2), (O4-Glc+), (O6A), (O15), (O16), (O18A), (O25B), and (O75), respectively, as shown in Table 1, wherein each n is independently an integer of 1 to 100, preferably of 3 to 50, for example 5 to 40, preferably of 5 to 30, for example 7 to 25, for example 10 to 20,
and wherein the ratio of O75 antigen polysaccharide to O6 antigen polysaccharide is about 1.2:1 to about 8:1, preferably about 1.5:1 to about 4:1, more preferably about 2:1.

In certain embodiments, the method further comprises administering to the subject from 1 to 15 additional E. coli antigen polysaccharides, each independently covalently linked to a carrier protein.

In a particular embodiment, each of the carrier proteins comprises the amino acid sequence of SEQ ID NO: 3.

In certain embodiments, the subject is a human having or at risk of having an E. coli (preferably ExPEC) infection, preferably an invasive ExPEC disease.

In certain embodiments, about 8-16 μg, preferably about 16 μg, of the O75 antigen polysaccharide is administered per administration.

In certain embodiments, the weight ratio of the administered E. coli antigen polysaccharides of O1:O2:O4:O6:O15:O16:O18:O25:O75 is 1:1:1:1:1:1:1:2:2.

In certain embodiments, the glucosylated O4 is a bioconjugate of an E. coli glucosylated O4 antigen polysaccharide covalently linked to a carrier protein, wherein the E. coli glucosylated O4 antigen polysaccharide comprises the structure of Formula (O4-Glc+) shown in Table 1, wherein n is an integer of 3 to 50, for example 5 to 40, preferably of 5 to 30, for example 7 to 25, for example 10 to 20,
wherein the bioconjugate of an E. coli glucosylated O4 antigen polysaccharide covalently linked to a carrier protein has been produced in an E. coli cell that comprises:
(i) a nucleotide sequence of an rfb gene cluster for the E. coli O4 antigen polysaccharide;
(ii) a nucleotide sequence encoding a glucosyl transferase having at least 80%, preferably at least 90%, preferably at least 95% sequence identity to SEQ ID NO: 4, wherein the glucosyl transferase is capable of modifying the E. coli O4 antigen polysaccharide to produce the E. coli glucosylated O4 antigen polysaccharide;
(iii) nucleotide sequences encoding a translocase and a glycosyltransferase having at least 80%, preferably at least 90%, preferably at least 95% sequence identity to SEQ ID NOs: 7 and 8 respectively, wherein the translocase is capable of translocating bactoprenol-linked glucose and the glycosyltransferase is capable of glucosylating bactoprenol;
(iv) a nucleotide sequence encoding the carrier protein; and
(v) a nucleotide sequence encoding an oligosaccharyl transferase capable of covalently linking the E. coli glucosylated O4 antigen polysaccharide to the carrier protein, preferably wherein the oligosaccharyl transferase is PglB from Campylobacter jejuni.

In one aspect, the invention also provides a composition comprising E. coli O1, O2, O4, O6, O15, O16, O18, O25 and O75 antigen polysaccharides (having the structures as indicated above for each antigen respectively), wherein each of the antigen polysaccharides is independently covalently linked to a carrier protein, and which composition does not comprise other E. coli O-antigen polysaccharides covalently linked to a carrier protein (i.e. a 9-valent composition). In certain embodiments of this aspect, the carrier protein is EPA. In other embodiments of this aspect, the carrier protein is CRM₁₉₇. In certain embodiments of this aspect, the covalent linkages are the result of chemical conjugation. In other embodiments, the covalent linkages are the result of bioconjugation. In certain embodiments of this aspect, the carrier protein is CRM₁₉₇ and the covalent linkages are the result of chemical conjugation. In other embodiments of this aspect, the carrier protein is EPA and the covalent linkages are the result of bioconjugation. In preferred embodiments of this aspect, the composition comprises an increased amount of E. coli O75 antigen polysaccharide as compared to O1, O2, or O6 antigen polysaccharide, preferably as compared to each one of O1, O2, and O6 antigen polysaccharide, preferably an about 1.5-4 times increased, preferably an about two times increased amount.

### EMBODIMENTS

Embodiment 1 is a composition comprising E. coli O75 antigen polysaccharide and at least one additional E. coli O-antigen polysaccharide selected from the group consisting of O1, O2, O4, O6, O15, O16, and O18, wherein each of the antigen polysaccharides is independently covalently linked to a carrier protein, and wherein the ratio of concentrations of O75 antigen polysaccharide to the additional O-antigen polysaccharide is about 1.2:1 to about 8:1, preferably about 1.5:1 to about 4:1, preferably about 1.5:1 to about 2.5:1, more preferably about 2:1.

Embodiment 1a is the composition of embodiment 1, wherein the at least one additional O-antigen polysaccharide is O1, preferably O1A.

Embodiment 1a1 is the composition of embodiment 1a, wherein the ratio of concentrations of O75 antigen polysaccharide to O1 antigen polysaccharide is 2:1.

Embodiment 1b is the composition of embodiment 1, wherein the at least one additional O-antigen polysaccharide is O2.

Embodiment 1b1 is the composition of embodiment 1b, wherein the ratio of concentrations of O75 antigen polysaccharide to O1 antigen polysaccharide is 2:1.

Embodiment 1c is the composition of embodiment 1, wherein the at least one additional O-antigen polysaccharide is O4, preferably glucosylated O4.

Embodiment 1c1 is the composition of embodiment 1c, wherein the ratio of concentrations of O75 antigen polysaccharide to O4 antigen polysaccharide is 2:1.

Embodiment 1d is the composition of embodiment 1, wherein the at least one additional O-antigen polysaccharide is O6, preferably O6A.

Embodiment 1d1 is the composition of embodiment 1d, wherein the ratio of concentrations of O75 antigen polysaccharide to O6 antigen polysaccharide is 2:1.

Embodiment 1e is the composition of embodiment 1, wherein the at least one additional O-antigen polysaccharide is O15.

Embodiment 1e1 is the composition of embodiment 1e, wherein the ratio of concentrations of O75 antigen polysaccharide to O15 antigen polysaccharide is 2:1.

Embodiment 1f is the composition of embodiment 1, wherein the at least one additional O-antigen polysaccharide is O16.

Embodiment 1f1 is the composition of embodiment 1f, wherein the ratio of concentrations of O75 antigen polysaccharide to O16 antigen polysaccharide is 2:1.

Embodiment 1g is the composition of embodiment 1, wherein the at least one additional O-antigen polysaccharide is O18, preferably O18A.

Embodiment 1g1 is the composition of embodiment 1g, wherein the ratio of concentrations of O75 antigen polysaccharide to O18 antigen polysaccharide is 2:1.

Embodiment 1h is the composition of embodiment 1, further comprising an O25 antigen polysaccharide independently covalently linked to a carrier protein, preferably the O25 antigen is O25B.

Embodiment 1h1 is the composition of embodiment 1h, wherein the ratio of concentrations of O75 antigen polysaccharide to O25 antigen polysaccharide is 1:1.

Embodiment 2 is the composition of embodiment 1, wherein the composition comprises the E. coli O75 antigen polysaccharide and two or more, preferably all, of E. coli O1, 02, O4, O6, O15, O16, O18, O25 antigen polysaccharides, wherein each of the antigen polysaccharides is independently covalently linked to a carrier protein, preferably the O1 antigen is O1A, the O4 is glucosylated, the O6 antigen is O6A, the O18 antigen is O18A, and the O25 antigen is O25B.

Embodiment 3 is the composition of embodiment 2, wherein
(i) the E. coli O1 antigen polysaccharide comprises the structure of Formula (O1A):

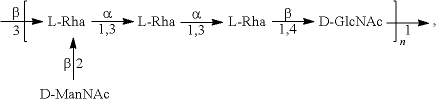

(ii) the E. coli O2 antigen polysaccharide comprises the structure of Formula (O2):

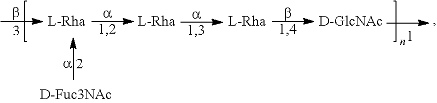

(iii) the E. coli O4 antigen polysaccharide comprises the structure of Formula (O4-Glc+):

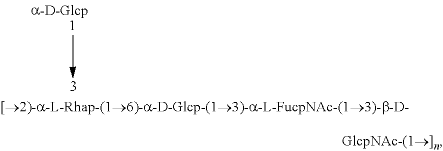

(iv) the E. coli O6 antigen polysaccharide comprises the structure of Formula (O6A):

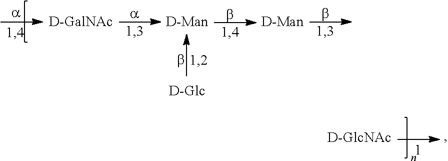

(v) the E. coli O15 antigen polysaccharide comprises the structure of Formula (O15):

[→2)-β-D-Galp-(1→3)-α-L-FucpNAc-(1→3)-β-D-GlcpNAc-(1→]ₙ,
(vi) the E. coli O16 antigen polysaccharide comprises the structure of Formula (O16):

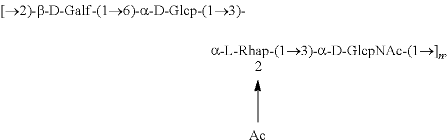

(vii) the E. coli O18 antigen polysaccharide comprises the structure of Formula (O18A):

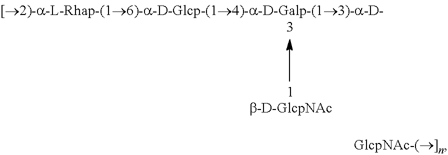

(viii) the E. coli O25 antigen polysaccharide comprises the structure of Formula (O25B):

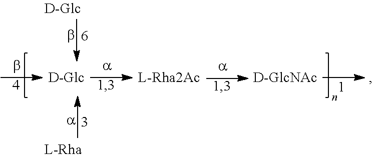

and
(ix) the E. coli O75 antigen polysaccharide comprises the structure of Formula (O75):

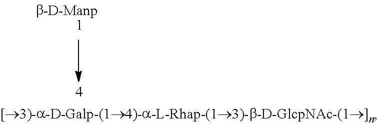

wherein each n is independently an integer of 1 to 100, preferably of 3 to 50, for example 5 to 40, preferably of 5 to 30, for example 7 to 25, for example 10 to 20.

Embodiment 4 is the composition of embodiment 2 or 3, wherein the weight ratio of the E. coli antigen polysaccharides of O1:O2:O4:O6:O15:O16:O18:O25:O75 is 1:1:1:1:1:1:1:2:2.

Embodiment 5 is the composition of any one of embodiments 1-4, wherein the concentration of the O75 antigen polysaccharide is from about 8 to about 50 μg/mL, preferably 12 to 40 μg/mL, e.g. 16-32 μg/mL, preferably about 26-38 μg/mL, preferably about 32 μg/mL.

Embodiment 6 is the composition of any one of embodiments 1-5, further comprising at least one other additional E. coli antigen polysaccharide covalently linked to a carrier protein.

Embodiment 7 is the composition of embodiment 6, wherein the one other additional E. coli antigen polysaccharide comprises O8 antigen polysaccharide with Formula (O8):

α-D-Manp3Me-(1→[3)-β-D-Manp-(1→2)-α-D-Manp-(1→2)-α-D-Manp-(1→]ₙ,
wherein n is an integer of 1 to 100, preferably of 3 to 50, for example 5 to 40, preferably of 5 to 30, for example 7 to 25, for example 10 to 20.

Embodiment 8 is the composition of any one of embodiments 1-7, wherein each carrier protein is independently selected from the group consisting of detoxified Exotoxin A of P. aeruginosa (EPA), E. coli flagellin (FliC), CRM₁₉₇, maltose binding protein (MBP), Diphtheria toxoid, Tetanus toxoid, detoxified hemolysin A of S. aureus, clumping factor A, clumping factor B, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin, Keyhole limpet hemocyanin (KLH), P. aeruginosa PcrV, outer membrane protein of Neisseria meningitidis (OMPC), and protein D from non-typeable Haemophilus influenzae.

Embodiment 9 is the composition of embodiment 8, wherein the carrier protein is detoxified exotoxin A of Pseudomonas aeruginosa (EPA) or CRM₁₉₇.

Embodiment 10 is the composition of any one of embodiments 1-9, wherein the carrier protein comprises 1 to 20, such as 1 to 10, or 2 to 4, glycosylation consensus sequences having the amino acid sequence of SEQ ID NO: 1, such as the consensus sequences having the amino acid sequence of SEQ ID NO: 2, most preferably the carrier protein comprises four of the glycosylation consensus sequences.

Embodiment 11 is the composition of any one of embodiments 1-10, wherein each carrier protein is EPA comprising the amino acid sequence of SEQ ID NO: 3.

Embodiment 12 is the composition of any one of embodiments 1-11, wherein the E. coli antigen polysaccharides are covalently linked to the carrier protein by bioconjugation or chemical conjugation, for example reductive amination chemistry (RAC), single-end conjugation, or conjugation with a (2-((2-oxoethyl)thio)ethyl) carbamate (eTEC) spacer.

Embodiment 13 is the composition of embodiment 12, wherein the E. coli antigen polysaccharides are covalently linked to the carrier protein by bioconjugation, preferably the polysaccharide is covalently linked to an Asn residue in a glycosylation site in the carrier protein.

Embodiment 14 is a composition comprising:
(i) a bioconjugate of an E. coli O1A antigen polysaccharide covalently linked to a detoxified Exotoxin A of P. aeruginosa (EPA-4) carrier protein, wherein the E. coli O1A antigen polysaccharide comprises the structure of Formula (O1A);
(ii) a bioconjugate of an E. coli O2 antigen polysaccharide covalently linked to an EPA-4 carrier protein, wherein the E. coli O2 antigen polysaccharide comprises the structure of Formula (O2);
(iii) a bioconjugate of an E. coli glucosylated O4 antigen polysaccharide covalently linked to an EPA-4 carrier protein, wherein the E. coli glucosylated O4 antigen polysaccharide comprises the structure of Formula (O4-Glc+);
(iv) a bioconjugate of an E. coli O6A antigen polysaccharide covalently linked to an EPA-4 carrier protein, wherein the E. coli O6A antigen polysaccharide comprises the structure of Formula (O6A);
(v) a bioconjugate of an E. coli O15 antigen polysaccharide covalently linked to an EPA-4 carrier protein, wherein the E. coli O15 antigen polysaccharide comprises the structure of Formula (O15);
(vi) a bioconjugate of an E. coli O16 antigen polysaccharide covalently linked to an EPA-4 carrier protein, wherein the E. coli O16 antigen polysaccharide comprises the structure of Formula (O16);
(vii) a bioconjugate of an E. coli O18A antigen polysaccharide covalently linked to an EPA-4 carrier protein, wherein the E. coli O18A antigen polysaccharide comprises the structure of Formula (O18A);
(viii) a bioconjugate of an E. coli O25B antigen polysaccharide covalently linked to an EPA-4 carrier protein, wherein the E. coli O25B antigen polysaccharide comprises the structure of Formula (O25B); and
(ix) a bioconjugate of an E. coli O75 antigen polysaccharide covalently linked to an EPA-4 carrier protein, wherein the E. coli O75 antigen polysaccharide comprises the structure of Formula (O75);
wherein the EPA-4 comprises the amino acid sequence of SEQ ID NO: 3,
wherein each of the structures of Formulas (O1A), (O2), (O4-Glc+), (O6A), (O15), (O16), (O18A), (O25B), and (O75) is shown in Table 1, wherein each n is independently an integer of 1 to 100, preferably of 3 to 50, for example 5 to 40, more preferably of 5 to 30, for example 7 to 25, for example 10 to 20, and
wherein the ratio of O75 antigen polysaccharide to O6A antigen polysaccharide is about 1.2:1 to about 8:1, preferably about 1.5:1 to about 4:1, more preferably about 2:1.

Embodiment 15 is the composition of embodiment 14, further comprising from 1 to 15 additional E. coli antigen polysaccharides each independently covalently linked to a carrier protein.

Embodiment 16 is the composition of any one of embodiments 2-15, wherein the glycosylated O4 is a bioconjugate of an E. coli glucosylated O4 antigen polysaccharide covalently linked to a carrier protein, wherein the E. coli glucosylated O4 antigen polysaccharide comprises the structure of Formula (O4-Glc+) shown in Table 1, wherein n is an integer of 3 to 50, for example 5 to 40, preferably of 5 to 30, for example 7 to 25, for example 10 to 20, wherein the bioconjugate of an E. coli glucosylated O4 antigen polysaccharide covalently linked to a carrier protein has been produced in an E. coli cell that comprises:
(i) a nucleotide sequence of an rfb gene cluster for the E. coli O4 antigen polysaccharide;
(ii) a nucleotide sequence encoding a glucosyl transferase having at least 80%, preferably at least 90%, preferably at least 95% sequence identity to SEQ ID NO: 4, wherein the glucosyl transferase is capable of modifying the E. coli O4 antigen polysaccharide to produce the E. coli glucosylated O4 antigen polysaccharide;
(iii) nucleotide sequences encoding a translocase and a glycosyltransferase having at least 80%, preferably at least 90%, preferably at least 95% sequence identity to SEQ ID NOs: 7 and 8 respectively, wherein the translocase is capable of translocating bactoprenol-linked glucose and the glycosyltransferase is capable of glucosylating bactoprenol;
(iv) a nucleotide sequence encoding the carrier protein; and
(v) a nucleotide sequence encoding an oligosaccharyl transferase capable of covalently linking the E. coli glucosylated O4 antigen polysaccharide to the carrier protein, preferably wherein the oligosaccharyl transferase is PglB from Campylobacter jejuni.

Embodiment 17 is a method of inducing an immune response to E. coli (preferably extra-intestinal pathogenic E. coli, ExPEC) in a subject, comprising administering to the subject the composition of any one of embodiments 1-16.

Embodiment 17a is the method of embodiment 17, wherein the subject is in need of said immune response.

Embodiment 18 is a method of inducing an immune response to E. coli (preferably extra-intestinal pathogenic E. coli, ExPEC) in a subject, comprising administering to the subject a first effective amount of E. coli O75 antigen polysaccharide, and a second effective amount of E. coli O6 antigen polysaccharide, wherein each of the antigen polysaccharides is independently covalently linked to a carrier protein, and wherein the ratio of the first effective amount to the second effective amount is about 1.2:1 to about 8:1, preferably about 1.5:1 to about 4:1, more preferably about 2:1.

Embodiment 18a is the method of embodiment 18, wherein the subject is in need of said immune response.

Embodiment 19 is the method of any one of embodiments 17-18a, wherein the immune response limits the severity of or prevents an invasive ExPEC disease in the subject.

Embodiment 20 is the method of embodiment 19, wherein the invasive ExPEC disease comprises sepsis and/or bacteremia.

Embodiment 20a is the method of embodiment 20, wherein the invasive ExPEC disease comprises sepsis.

Embodiment 20b is the method of embodiment 20, wherein the invasive ExPEC disease comprises bacteremia.

Embodiment 21 is the method of any one of embodiments 18-20, further comprising administering to the subject one or more, preferably all, of E. coli O1, O2, O4, O15, O16, O18, O25 antigen polysaccharides, wherein each of the antigen polysaccharides is independently covalently linked to a carrier protein,
preferably the O1 antigen is O1A, the O4 is glucosylated, the O6 antigen is O6A, the O18 antigen is O18A, and the O25 antigen is O25B,
more preferably each of the O1A, O2, glucosylated O4, O6A, O15, O16, O18A, O25B, and O75 antigen polysaccharides comprise the structures of Formulas (O1A), (O2), (O4-Glc+), (O6A), (O15), (O16), (O18A), (O25B), and (O75), respectively, as shown in Table 1, wherein each n is independently an integer of 1 to 100, preferably of 3 to 50, for example 5 to 40, preferably of 5 to 30, for example 7 to 25, for example 10 to 20, and wherein the ratio of O75 antigen polysaccharide to O6 antigen polysaccharide is about 1.2:1 to about 8:1, preferably about 1.5:1 to about 4:1, more preferably about 2:1.

Embodiment 22 is the method of any one of embodiments 18-21, further comprising administering to the subject from 1 to 15 additional E. coli antigen polysaccharides, each independently covalently linked to a carrier protein.

Embodiment 23 is the method of any one of embodiments 18-22, wherein each of the carrier proteins comprises the amino acid sequence of SEQ ID NO: 3.

Embodiment 24 is the method of any one of embodiments 17-23, wherein the subject is a human having or at risk of having an E. coli (preferably ExPEC) infection or an invasive ExPEC disease.

Embodiment 25 is the method of any one of embodiments 17-24, wherein about 8-16 μg, preferably about 16 μg, of the O75 antigen polysaccharide is administered per administration.

Embodiment 26 is the method of any one of embodiments 21-25, wherein the weight ratio of the administered E. coli antigen polysaccharides of O1:O2:O4:O6:O15:O16:O18:O25:O75 is 1:1:1:1:1:1:1:2:2.

Embodiment 27 is the method of any one embodiments 21-26, wherein the glycosylated O4 is a bioconjugate of an E. coli glucosylated O4 antigen polysaccharide covalently linked to a carrier protein, wherein the E. coli glucosylated O4 antigen polysaccharide comprises the structure of Formula (O4-Glc+) shown in Table 1, wherein n is an integer of 3 to 50, for example 5 to 40, preferably of 5 to 30, for example 7 to 25, for example 10 to 20, wherein the bioconjugate of an E. coli glucosylated O4 antigen polysaccharide covalently linked to a carrier protein has been produced in an E. coli cell that comprises:
(i) a nucleotide sequence of an rfb gene cluster for the E. coli O4 antigen polysaccharide;
(ii) a nucleotide sequence encoding a glucosyl transferase having at least 80%, preferably at least 90%, preferably at least 95% sequence identity to SEQ ID NO: 4, wherein the glucosyl transferase is capable of modifying the E. coli O4 antigen polysaccharide to produce the E. coli glucosylated O4 antigen polysaccharide;
(iii) nucleotide sequences encoding a translocase and a glycosyltransferase having at least 80%, preferably at least 90%, preferably at least 95% sequence identity to SEQ ID NOs: 7 and 8 respectively, wherein the translocase is capable of translocating bactoprenol-linked glucose and the glycosyltransferase is capable of glucosylating bactoprenol;
(iv) a nucleotide sequence encoding the carrier protein; and
(v) a nucleotide sequence encoding an oligosaccharyl transferase capable of covalently linking the E. coli glucosylated O4 antigen polysaccharide to the carrier protein, preferably wherein the oligosaccharyl transferase is PglB from Campylobacter jejuni.

Embodiment 27 is the composition of any one of embodiments 1-16 for use in a method of inducing an immune response to E. coli (preferably extra-intestinal pathogenic E. coli, ExPEC) in a subject.

Embodiment 28 is the use of the composition of any one of embodiments 1-16 in the manufacture of a medicament for inducing an immune response to E. coli (preferably extra-intestinal pathogenic E. coli, ExPEC) in a subject.

Embodiment 29 is a combination of a first effective amount of E. coli O75 antigen polysaccharide and a second effective amount of E. coli O6 antigen polysaccharide for use in a method of inducing an immune response to E. coli, preferably extra-intestinal pathogenic E. coli (ExPEC), in a subject, wherein each of the antigen polysaccharides is independently covalently linked to a carrier protein, and wherein the ratio of the first effective amount to the second effective amount is about 1.2:1 to about 8:1, preferably about 1.5:1 to about 4:1, more preferably about 2:1.

Embodiment 29a is the combination of embodiment 29, wherein the first effective amount and the second effective amount are in the same composition.

Embodiment 29b is the combination of embodiment 29, wherein the first effective amount and the second effective amount are in separate compositions.

Embodiment 29c is the combination of any one of embodiments 29-29b, wherein the subject is in need of said immune response.

Embodiment 30 is the combination of any one of embodiments 29-29c, wherein the immune response limits the severity of or prevents an invasive ExPEC disease in the subject.

Embodiment 31 is the combination of embodiment 30, wherein the invasive ExPEC disease comprises sepsis and/or bacteremia.

Embodiment 32 is the combination of any one of embodiments 29-31, further comprising one or more, preferably all, of effective amounts of E. coli O1, O2, O4, O15, O16, O18, O25 antigen polysaccharides, wherein each of the antigen polysaccharides is independently covalently linked to a carrier protein, preferably the O1 antigen is O1A, the O4 is glucosylated, the O6 antigen is O6A, the O18 antigen is O18A, and the O25 antigen is O25B, preferably each of the O1A, O2, glucosylated O4, O6A, O15, O16, O18A, O25B, and O75 antigen polysaccharides comprise the structures of Formulas (O1A), (O2), (O4-Glc+), (O6A), (O15), (O16), (O18A), (O25B), and (O75), respectively, as shown in Table 1, wherein each n is independently an integer of 1 to 100, preferably of 3 to 50, for example 5 to 40, preferably of 5 to 30, for example 7 to 25, for example 10 to 20, and wherein the ratio of O75 antigen polysaccharide to O6 antigen polysaccharide is about 1.2:1 to about 8:1, preferably about 1.5:1 to about 4:1, more preferably about 2:1.

Embodiment 33 is the combination of any one of embodiments 29-32, further comprising effective amounts of from 1 to 15 additional E. coli antigen polysaccharides, each independently covalently linked to a carrier protein.

Embodiment 34 is the combination of any one of embodiments 29-33, wherein each of the carrier proteins comprises the amino acid sequence of SEQ ID NO: 3.

Embodiment 35 is the combination of any one of embodiments 29-34, wherein the subject is a human having or at risk of having an E. coli (preferably ExPEC) infection or an invasive ExPEC disease.

Embodiment 36 is the combination of any one of embodiments 29-35, wherein the effective amount of the O75 antigen polysaccharide is about 8-16 μg, preferably about 16 μg.

Embodiment 37 is a combination of a first effective amount of E. coli O75 antigen polysaccharide and a second effective amount of E. coli O6 antigen polysaccharide in the manufacture of a medicament for inducing an immune response to E. coli, preferably extra-intestinal pathogenic E. coli (ExPEC), in a subject, wherein each of the antigen polysaccharides is independently covalently linked to a carrier protein, and wherein the ratio of the first effective amount to the second effective amount is about 1.2:1 to about 8:1, preferably about 1.5:1 to about 4:1, more preferably about 2:1.

Embodiment 37a is the combination of embodiment 37, wherein the subject is in need of said immune response.

Embodiment 38 is the combination of embodiment 37 or 37a, wherein the immune response limits the severity of or prevents an invasive ExPEC disease in the subject.

Embodiment 39 is the combination of embodiment 38, wherein the invasive ExPEC disease comprises sepsis and/or bacteremia.

Embodiment 40 is the combination of any one of embodiments 37-39, further comprising one or more, preferably all, of effective amounts of E. coli O1, O2, O4, O15, O16, O18, O25 antigen polysaccharides in the manufacture of the medicament, wherein each of the antigen polysaccharides is independently covalently linked to a carrier protein, preferably the O1 antigen is O1A, the O4 is glucosylated, the O6 antigen is O6A, the O18 antigen is O18A, and the O25 antigen is O25B, preferably each of the O1A, O2, glucosylated O4, O6A, O15, O16, O18A, O25B, and O75 antigen polysaccharides comprise the structures of Formulas (O1A), (O2), (O4-Glc+), (O6A), (O15), (O16), (O18A), (O25B), and (O75), respectively, as shown in Table 1, wherein each n is independently an integer of 1 to 100, preferably of 3 to 50, for example 5 to 40, preferably of 5 to 30, for example 7 to 25, for example 10 to 20, and wherein the ratio of O75 antigen polysaccharide to O6 antigen polysaccharide is about 1.2:1 to about 8:1, preferably about 1.5:1 to about 4:1, more preferably about 2:1.

Embodiment 41 is the combination of any one of embodiments 37-40, further comprising effective amounts of from 1 to 15 additional E. coli antigen polysaccharides, each independently covalently linked to a carrier protein, in the manufacture of the medicament.

Embodiment 42 is the combination of any one of embodiments 37-41, wherein each of the carrier proteins comprises the amino acid sequence of SEQ ID NO: 3.

Embodiment 43 is the combination of any one of embodiments 37-42, wherein the subject is a human having or at risk of having an E. coli (preferably ExPEC) infection or an invasive ExPEC disease.

Embodiment 44 is the combination of any one of embodiments 37-43, wherein the effective amount of the O75 antigen polysaccharide in one dose for administration to the subject is about 8-16 μg, preferably about 16 μg.

### EXAMPLES

### Example 1: Epidemiological Data of E. coli Infections

To determine the O-serotype distribution of bacteremia-causing E. coli, global surveillance studies were performed. Between 2011 and 2017, more than 3200 E. coli bloodstream isolates were collected from patients≥60 years of age hospitalized in countries within North America, Europe, the Asia-Pacific region, and South America. Each strain was analyzed for O antigen serotype using classical agglutination techniques and sequence-based O-genotyping. See Table 2 below.

Isolated human blood samples were analyzed to determine the identity of pathogens therein and their antibiotic resistance patterns. E. coli isolates were obtained from the samples following the analysis. E. coli identity was verified by MALDI-TOF MS. Further analysis on the E. coli isolates was performed using an antisera-based agglutination assay to determine their O-antigen serotype (DebRoy et al. (2011) Animal health research reviews/Conference of Research Workers in Animal Diseases 12, 169-185). Isolates un-typeable by the agglutination method, were further analyzed by whole-genome sequencing followed by O-genotyping based on O-serotype specific wzy and wzx gene sequences.

TABLE 2
Distribution of the most common bacteremia-associated E. coli
O-serotypes from a collection of 3217 blood isolates collected
globally between 2011 and 2017, based on O-serotyping by agglutination
plus O-genotyping of isolates un-typeable by agglutination. Subjects
were hospitalized in the following countries: USA, Canada, Argentina,
Brazil, UK, Germany, Spain, Italy, The Netherlands, France, Japan,
Thailand, South Korea and Australia.

| O-serotype | Prevalence n (%) |
|---|---|
| O25 | 737 (22.9%) |
| O2 | 268 (8.3%) |
| O6 | 261 (8.1%) |
| O1 | 255 (7.9%) |
| O75 | 145 (4.5%) |
| O15 | 110 (3.4%) |
| O8 | 104 (3.2%) |
| O16 | 103 (3.2%) |
| O4 | 96 (3.0%) |
| O18 | 91 (2.8%) |

Stratification of on geographical location in the global set of bacteremia-associated E. coli showed a prevalence of the top 10 O-serotypes independent of location, suggesting these to be the predominant O-serotypes globally associated with bacteremia-causing E. coli.

In the global set of bacteremia-associated multi-drug resistant E. coli isolates (n=345), i.e. those strains that are resistant to at least three classes of clinically relevant antimicrobial drugs, the prevalence of the top 10 O-serotypes is 75.4%.

All information from epidemiology analysis taken together, the 10 predominant O-serotypes could cover an estimated 60-80% of E. coli-associated bacteremia infections, assuming coverage of subportions of the un-typeable strains.

A multivalent vaccine covering a significant proportion of bacteremia-causing E. coli serotypes would be very useful. The O-serotypes of Table 2 would thus be good candidates for an O-antigen based multivalent vaccine. Such a vaccine could beneficially be prepared using chemical conjugation or bioconjugation technology.

### Example 2: Production of E. coli O-Antigen Bioconjugates and Resulting Bioconjugate Products

Ten (10) bioconjugates were produced, including E. coli O1A-EPA bioconjugate, O2-EPA bioconjugate, O4-Glc+-EPA bioconjugate (also referred to as O4-EPA hereinbelow, i.e. in the examples below the bioconjugate of the O4 antigen polysaccharide is the variant wherein O4 is glucosylated, having glycan structure (O4-Glc+) shown in Table 1; description of making this variant using a novel and hitherto unreported and unknown glucosyltransferase GtrS that is capable of specifically modifying an E. coli O4 antigen polysaccharide by addition of glucose to produce the E. coli glucosylated O4 antigen was provided in detail in PCT/US20/23404, filed on 18 Mar. 2020, incorporated by reference herein), O6A-EPA bioconjugate, O8-EPA bioconjugate, O15-EPA bioconjugate, O16-EPA bioconjugate, O18A-EPA bioconjugate, O25B-EPA bioconjugate, and O75-EPA bioconjugate. The structures of the glycans of these conjugates can be seen in the respective Formulas in Table 1. A composition comprising the 10 bioconjugates is referred to herein as ‘ExPEC10V’. A composition comprising the O1A-EPA, O2-EPA, O6A-EPA and O25B-EPA bioconjugates is referred to as ‘ExPEC4V’ (and was previously described in, for example, WO 2015/124769 and WO 2017/035181). Production of these ten bioconjugates, and exemplary production strains for these bioconjugates, were described in detail in PCT/US20/23404, filed on 18 Mar. 2020, incorporated by reference herein. Escherichia coli W3110 Parental Strain

The non-pathogenic E. coli K12 strain W3110 was used as the parental strain for the construction of all ten production strains. The E. coli K12 strain W3110 was obtained from the Coli Genetic Stock Center (Yale University, New Haven (CT), USA, product number CGSC #4474). Its relevant genotype was previously described (E. coli W3110, F-, lambda-, IN(rrnD-rrnE)1, rph-1) and its genomic sequence was previously published (Hayashi K, et al., 2006, Mol. Syst. Biol. 2006.0007 (doi:10.1038/msb4100049). The E. coli W3110 strain was genetically modified to enable production of each of the E. coli O-antigen bioconjugates (Table 3).

### Bioconjugate Production Strains

The “ExPEC4V” and “ExPEC10V” compositions both comprise the O2-EPA and O25B-EPA bioconjugates from the same production strains. The “ExPEC4V” composition comprises the O1A-EPA bioconjugate from the stGVXN4411 or stLMTB10217 production strains, while the “ExPEC10V” composition comprises the O1A-EPA bioconjugate from the stLMTB10217 production strain. The “ExPEC4V” composition comprises the O6A-EPA bioconjugate from the stGVXN4112 production strain, while the “ExPEC10V” composition comprises the O6A-EPA bioconjugate from the stLMTB10923 production strain. Furthermore, the “ExPEC10V” composition comprises the O4-EPA (i.e. (O4-Glc+)-EPA), O8-EPA, O15-EPA, O16-EPA, O18A-EPA, and O75-EPA bioconjugates from production strains that are not used for “ExPEC4V”. Different production strains could vary in the plasmids for expression of the EPA carrier protein and/or the oligosaccharyl transferase PglB, as indicated below. An overview of several exemplary production strains is given in Table 3 below.

TABLE 3
Overview of genetic engineering of E. coli production strains for
O-antigen bioconjugates for ExPEC4V and ExPEC10V vaccine compositions

| | | Genomic mutations | | | Plasmids | |
|---|---|---|---|---|---|---|
| Serotype | Strain name | rfb gene cluster | waaL | gtrABS | pglB | epa |
| O1A | stGVXN4411 | Δrfb::O1A rfb | ΔwaaL | — | pGVXN970 | PGVXN1076 |
| (ExPEC4V) | | upecGVXN_032 | | | | |
| O1A | stLMTB10217 | Δrfb::O1A rfb | ΔwaaL | — | pGVXN1221 | PGVXN1076 |
| (ExPEC4V; | | upecGVXN_032 | | | | |
| ExPEC10V) | | | | | | |
| O2 | stGVXN4906 | Δrfb::O2 rfb | Δwaal | — | pGVXN971 | pGVXN1076 |
| | | upecGVXN_116 | | | | |
| O4 | BVEC-L-00684 | Δrfb::O4 rfb | Δwaal | ΔgtrS::gtrS | pGVXN1217 | pGVXN1076 |
| | | CCUG11450 | | O4 | | |
| O6A | stGVXN4112 | Δrfb::O6A rfb | Δwaal | — | pGVXN114 | pGVXN659 |
| (ExPEC4V) | | CCUG11309 | | | | |
| O6A | stLMTB10923 | Δrfb::O6A rfb | Δwaal | — | pGVXN1221 | pGVXN1076 |
| (ExPEC10V) | | CCUG11309 | | | | |
| O8 | stLMTB11734 | Δrfb::O8 rfb | Δwaal | ΔgtrABS | pGVXN970 | pGVXN1076 |
| | | E2420 | | | | |
| O15 | stLMTB11738 | Δrfb::O15 rfb | Δwaal | ΔgtrABS | pGVXN1221 | pGVXN1076 |
| | | OC24891 | | | | |
| O16 | stLMTB11739 | Δrfb::O16 rfb | Δwaal | ΔgtrABS | pGVXN2381 | pGVXN1076 |
| | | OC24208 | | | | |
| O18A | BVEC-L-00559 | Δrfb::O18A rfb | Δwaal | ΔgtrABS | pGVXN970 | pGVXN1076 |
| | | OC24255 | | | | |
| O25B | stGVXN4459 | Δrfb::O25B rfb | Δwaal | ΔgtrABS | pGVXN970 | pGVXN1076 |
| | | upecGVXN_138 | | | | |
| O75 | stLMTB11737 | Δrfb::O75 rfb | Δwaal | ΔgtrABS | pGVXN1217 | pGVXN1076 |
| | | CCUG31 | | | | |

Alternative production strains were also prepared and used for some of the bioconjugates, e.g. to find clones with improved yields and/or changes in certain characteristics of the bioconjugates.

O-Antigen Biosynthesis (rfb) Gene Cluster

In all E. coli O-antigen production strains, the naturally occurring E. coli W3110 genomic O16::IS5-antigen biosynthesis (rfb) gene cluster was replaced by the selected O-antigen-specific biosynthesis clusters from E. coli strains of the selected serotype, encoding for the serotype-specific O-antigen structures (see Table 1 for these O-antigen structures). The ten donor rfb clusters were selected or confirmed after whole-genome analysis of E. coli blood isolates. Replacement of the W3110 O16::IS5 rfb gene cluster, which is defective in O-antigen biosynthesis, has been achieved in a single homologous recombination event. In case of the O16 and O18A rfb gene clusters, the donor DNA recombined via the flanking gnd and rmlCA genes, while the rfb gene cluster for the other strains recombined via the flanking gnd and galF genes. Sequences of the rfb clusters in the production strains are provided in SEQ ID NOs: 9 and 11-19.

O-Antigen Ligase (waaL) Gene

All E. coli O-antigen production strains carry an artificially introduced deletion of the E. coli W3110 genomic O-antigen ligase encoded by the waaL gene. In the ΔwaaL strains the transfer of the O-antigen to lipid A is disrupted, which instead directs transfer of the O-antigen to the carrier protein to increase product yield.

O-Antigen Glucosylation (gtrABS) Genes

In the E. coli O8, O15, O16, O18A, O25B, and O75 production strains the E. coliW3110 genomic gtrABS genes, which are responsible for O16 O-antigen glucosylation, have been deleted. While the gtrA and gtrB genes in different serotypes are highly homologous and interchangeable, the gtrS gene encodes a serotype-specific O-antigen glycosyl transferase. In E. coli W3110 GtrS can transfer a glucose (Glc) residue to the GlcNAc sugar in the α-L-Rha-(1→3)-D-GlcNAc motif of the E. coli O16 O-antigen. In the E. coli O1A, O2 and O6A production strains no deletion or replacement of the gtrABS gene has occurred. These O-antigens miss the α-L-Rha-(1--3)-D-GlcNAc motif that is the natural substrate for E. coli O16 gtrS. In the E. coli O4 production strain, the W3110 gtrS gene has been replaced with the E. coli O4 gtrS gene to accommodate proper glucosylation of the E. coli O4 O-antigen (a coding sequence of the E. coli O4 gtrS gene is provided herein as SEQ ID NO: 5 and an amino acid sequence of E. coliO4 GtrS protein is provided herein as SEQ ID NO. 4; see e.g. also PCT/US20/23404, filed on 18 Mar. 2020).

### Oligosaccharyl Transferase PglB

All E. coli O-antigen production strains expressed a variant of the C. jejuni glycosyl transferase PglB, which can transfer the O-antigen onto an amino acid consensus sequence on a carrier protein by N-glycosylation. PglB has broad substrate recognition, but due to low product yields several production strains were prepared expressing a PglB variant having modified substrate specificities, which resulted in improved product yield (see e.g. WO 2016/107818, WO 2016/107819). The pglB gene was placed behind an Isopropyl β-D-1-thiogalactopyranoside (IPTG) inducible promoter on a plasmid. Table 4 below lists the PglB variants encoded by the plasmids used for production of the E. coli O-antigen production strains for the bioconjugates for the ExPEC4V and ExPEC10V compositions described above. Further plasmids with variation in vector backbone, antibiotic resistance marker, and/or alternative PglB variants have also been tested successfully for bioconjugate production.

TABLE 4
PglB and EPA plasmids used in E. coli O-antigen Production Strains

| Plasmid | | |
|---|---|---|
| name | Gene | Description¹ |
| pGVXN114 | pglB | C. jejuni codon usage; |
| | | SpR |
| pGVXN970 | pglB | E. coli codon usage |
| | | optimized; SpR |
| pGVXN971 | pglB^{N534Q} | E. coli codon usage |
| | | optimized; The natural |
| | | glycosylation site of PglB |
| | | was inactivated; SpR |
| pGVXN1217 | pglB^{N311V} | E. coli codon usage |
| | | optimized; Substrate |
| | | optimized PglB; SpR |
| pGVXN1221 | pglB^{N311V,K482R,D483H,A669V} | E. coli codon usage |
| | | optimized; Substrate |
| | | optimized PglB; SpR |
| pGVXN2381 | pglB^{Y77H,S80R,Q287P,K289R,N311V} | E. coli codon usage |
| | | optimized; Substrate |
| | | optimized PglB; SpR |
| pGVXN659 | EPA-4 | EPA with four |
| | | bioconjugation sites; |
| | | AmpR |
| pGVXN1076 | EPA-4 | EPA with four |
| | | bioconjugation sites; |
| | | KanR |

¹SpR, spectinomycin resistant; AmpR, ampicillin resistant; KanR, kanamycin resistant
Optimal PglB variants for each bioconjugate of the ten E. coli O-antigens in ExPEC10V were determined as described in more detail in e.g. PCT/US20/23415, filed on 18 Mar. 2020, incorporated in its entirety by reference herein.

### Carrier Protein (EPA)

All E. coli O-antigen production strains expressed a genetically detoxified P. aeruginosa ADP-ribosyltransferase toxoid (EPA) as a carrier protein for the O-antigen. The EPA toxoid differs from wild-type EPA toxin in two residues: Leu552 was changed to Val and Glu553 (in the catalytic domain) was deleted. Glu553 deletions were reported to significantly reduce toxicity. In addition to the detoxification mutation, four (EPA-4) consensus N-glycosylation site motifs were introduced. The epa gene was placed behind a 1-Arabinose (Ara) inducible promoter on a plasmid (Table 4). Table 4 is limited to the plasmids used in production strains for bioconjugates used in the “ExPEC4V” and “ExPEC10V” compositions described above. Plasmids with variation in vector backbone, antibiotic resistance marker, and/or EPA variants, e.g. varying in the number of consensus N-glycosylation site motifs (e.g. having two such motifs, EPA-2), have also been tested successfully for bioconjugate production.

### Example 3: Optimizing the Oligosaccharyltransferase for Generation of Bioconjugates of E. coli O-Antigens

Yield optimization for bioconjugate production can be achieved by modification of the C. jejuni oligosaccharyl transferase PglB, which can lead to a more efficient or higher degree of N-glycosylation of the O-antigen of interest to the EPA carrier protein. In an E. coli strain for production of bioconjugate with glucosylated O4 (O4-Glc+) O-antigen polysaccharide, such optimization strategy was applied and resulted in an (O4-Glc+)-specific optimized PglB variant improving bioconjugate product yield.

In this approach, an O4-Glc+O-antigen polysaccharide producing strain containing an EPA-expression plasmid was transformed with a variety of different PglB expression plasmids, each of which contained different amino acid substitutions in the PglB protein, altering substrate specificity. Bioconjugate production level and profile of each strain was assessed at shake-flask level in osmotic shock experiments, and readout was performed by capillary electrophoresis immunoassays on the periplasmic extract using O4-Glc+-specific monoclonal antibodies.

One of the tested PglB variants containing an N311V amino acid substitution was found to improve product yield of glucosylated O4 bioconjugates significantly (see PCT/US20/23415, filed on 18 Mar. 2020, incorporated in its entirety by reference herein).

In a further improvement where the N311V PglB-variant was further modified, an Y77H amino acid substitution further enhanced O4-Glc+-specific product yield and showed an increased degree of di- and tri-glycosylated product compared to the N311V PglB-variant, where other modifications were found to be neutral or had a negative effect on product yield (see PCT/US20/23415, filed on 18 Mar. 2020, incorporated in its entirety by reference herein). Plasmid pLMTB4008 (SpR) encodes E. coli codon usage optimized, (O4-Glc+)-substrate optimized, PglB variant with mutations Y77H and N311V.

The PglB variant with optimized substrate specificity for O4-Glc+O-antigen polysaccharide, containing N311V and Y77H amino acid substitutions relative to wild-type (wt) C. jejuni glycosyl transferase PglB, was found to double bioconjugate yield compared to the first round optimized PglB-N311V variant.

Similarly using screens, the most optimal yielding PglB variants were also determined for E. coli O-antigen bioconjugate production of the other nine serotypes in the ExPEC10V composition, see e.g. PCT/US20/23415, filed on 18 Mar. 2020, incorporated in its entirety by reference herein.

For bioconjugates having the O1A, O6A, or O15 antigen polysaccharide, PglB with amino acid mutations N311V, K482R, D483H, and A669V was found to give the highest yields.

For bioconjugates having the O2, O8, O18A, or O25B antigen polysaccharide, wild-type PglB (i.e. not having amino acid mutations at positions 77, 80, 287, 289, 311, 482, 483 and 669) was found to give the highest yields.

For bioconjugates having the O16 antigen polysaccharide, PglB with amino acid mutations Y77H, S80R, Q287P, K289R, and N311V was found to give the highest yields.

For bioconjugates having the O75 antigen polysaccharide, PglB with amino acid mutation N311V was found to give the highest yields.

These results showed that the optimal PglB variant is different for different O-antigens, and that the optimal PglB variant for producing a bioconjugate with a given O-antigen polysaccharide is unpredictable.

### Example 4: Quality attributes of Bioconjugates of O-Antigens from 10 E. coli Serotypes

O-glycan residues of the target O-antigens are structurally diverse and have variable repeating units. The specificity and affinity of the glycosyl transferase PglB is linked to the glycan structure. Thus, making a bioconjugate that has the desired quality attributes, e.g., purity, glycan/protein ratio, etc., is a challenging, non-straightforward, task. The right combination of PglB and EPA carrier protein determines the yield and may influence glycosylation efficiency. By optimizing the PglB and carrier proteins, bioconjugates having the desired quality attributes were produced. It may be also important to maintain a lower threshold value of total carrier protein, particularly when one or more O-antigen bioconjugates are combined together and administered in a single composition or vaccine, because very high amounts of carrier protein may lead to immunological interference. In order to avoid such a phenomenon, conjugates having a higher glycan/protein ratio are preferred. Hence, for ExPEC10V vaccine, bioconjugates with at least comparable (to the previously described ExPEC4V vaccine that has been subject to clinical trials) glycosylation ratio were developed.

The bioconjugates were each produced by culturing the respective host cells (Example 2, Table 3) in bioreactors (10 L and/or 200 L volumes) and expression of the bioconjugates, following methods previously described. Each drug substance was manufactured batch-wise by bacterial fed-batch fermentation to generate biomass containing the expressed bioconjugates of the corresponding polysaccharide serotype. Cells were cultured and induced with IPTG and arabinose. The bioconjugates were isolated from the periplasm of the cells in the bioreactor cultures by osmotic shock followed by chromatographic purification. This process was performed for each of the 10 bioconjugates.

The E. coli O-antigen bioconjugates thus prepared that are drug substances (DSs) for ExPEC10V and ExPEC4V showed comparable critical quality attributes: (1) process-related purity (measured by RP-HPLC) was higher than 95%, (2) polysaccharide/protein ratio ranged between about 0.1-0.5, mostly between 0.15 and 0.45, (3) bacterial endotoxin (Ph. Eur. 2.2.3) was less than 0.5 EU/μg polysaccharide. The average length of the individual polysaccharide chains was typically between about 10-20 repeating units (measured using high resolution SDS-PAGE).

The structures of the polysaccharide repeat units were confirmed (by NMR and MS/MS of the conjugates, intact or trypsin-digested) to be the ones shown in the Formulas for the corresponding serotypes in Table 1, for all ten bioconjugates that are DSs for the ExPEC10V composition described above.

ExPEC10V drug product (DP) comprises a mixture of the ten monovalent DSs described above.

### Example 5: Toxicology of ExPEC10V Vaccine

A single-dose pilot toxicity and local tolerance study (non-GLP) with ExPEC10V was conducted in female NZW rabbits. One group (n=2) received an intramuscular (IM) injection (on Day 0) of the control (saline), and a second group (n=4) received an IM injection of ExPEC10V at 105.6 μg total polysaccharide (PS)/dose (9.6:9.6:9.6:9.6:9.6:9.6:9.6:9.6:19.2:9.6 μg PS per dose, for respectively O-serotypes O1A, O2, O4, O6A, O8, O15, O16, O18A, O25B and O75) using a dosing volume of 0.6 mL (176 μg PS/mL). Necropsy was performed on Day 2.

There were no mortalities observed. In addition, there were no vaccine-related effects noted for clinical observations (including injection site effects using Draize scoring), body weight, food consumption, and body temperature. Histopathologically, there were no vaccine-related changes observed at the administration site or draining (iliac) lymph node. A minimal increase in germinal center formation in the spleen was observed in one out of four treated animals (Day 2), and was considered a normal, immunological response to the injected vaccine. Overall, the administration of a single IM dose of ExPEC10V to female rabbits was well-tolerated.

### Example 6: Immunogenicity of ExPEC10V Blended Formulation in Rabbits

An ExPEC4V vaccine (comprising bioconjugates of E. coli O1A, O2, O6A, and O25B serotypes) has previously been shown to be immunogenic for these four serotypes in rats, rabbits, and humans (see e.g. WO 2015/124769; WO 2017/035181; Huttner et al, 2017, Lancet Infect Dis, http://dx.doi.org/10.1016/S1473-3099(17)30108-1; R W Frenck Jr, et al, 2019, Lancet Infect Dis 19(6): 631-640, http://dx.doi.org/10.1016/S1473-3099(18)30803-X). Immunogenicity of the bioconjugates of E. coli serotypes O4-Glu+, O8, O15, O16, O18A, and O75 (all having EPA-2 as carrier protein) when separately administered (monovalent) to rats confirmed that also each of these bioconjugates was immunogenic, since ELISA data indicated that each of these bioconjugates could elicit high levels of E. coli O-antigen specific antibodies (not shown).

Immunogenicity of the 10-valent vaccine that contained a mixture of the 10 bioconjugates as described above was also tested. New Zealand White (NZW) rabbits (female, 12-16 weeks old) received 3 intramuscular immunizations with ExPEC10V or saline administered 2 weeks apart (Table 5; administration at days 0, 14, and 27). The 10 polysaccharides that are part of the ExPEC10V vaccine used in these experiments were conjugated to the carrier protein EPA containing 4 sites of glycosylation (EPA-4). The vaccine was formulated in 3 different doses: Group 1 (‘high dose’): 8 μg/dose of O1A, O2, O6A, O4, O8, O15, O16, O18 and O75 and 16 μg/dose of O25B; Group 2 (‘medium dose’): 4 μg/dose of O2, O4, O8, O15, O16, O18 and O75, 8 μg/dose of O1A and O6A and 16 μg/dose of O25B; Group 3 (‘low dose’): 0.4 μg/dose of O2, O4, O8, O15, O16, O18 and O75, 0.8 μg/dose of O1A and O6A and 1.6 μg/dose of O25B. Animals from the control group (Group 4) received only saline (0.9% (w/v) sodium chloride solution) (Table 5).

Antibody responses were evaluated at day 0 (pre-immunization) and days 14, 27 and 42 post-immunization. Serum antibody levels induced by each of the bioconjugates included in the vaccine and the carrier protein EPA were measured by ELISA (total IgG), using type-specific LPS as coating material. The antibody titers were reported as EC50 values that correspond to the half maximal effective concentration based on duplicates of 12-step titration curves plotted in a 4-parameter logistic nonlinear regression model. Functional activity was determined by OPK.

TABLE 5
Description of experimental groups.

| Experi- | | |
|---|---|---|
| mental | Dosing (μg/PS) | Sample |
| groups | O1A:O2:O6A:O25B:O4:O8:O15:O16:O18A:O75 | size |
| Group 1 | 8:8:8:16:8:8:8:8:8:8 | 7 |
| (high dose) | | |
| Group 2 | 8:4:8:16:4:4:4:4:4:4 | 7 |
| (medium | | |
| dose) | | |
| Group 3 | 0.8:0.4:0.8:1.6:0.4:0.4:0.4:0.4:0.4:0.4 | 7 |
| (low dose) | | |
| Group 4 | 0.9% (w/v) sodium chloride solution | 7 |
| (control) | | |

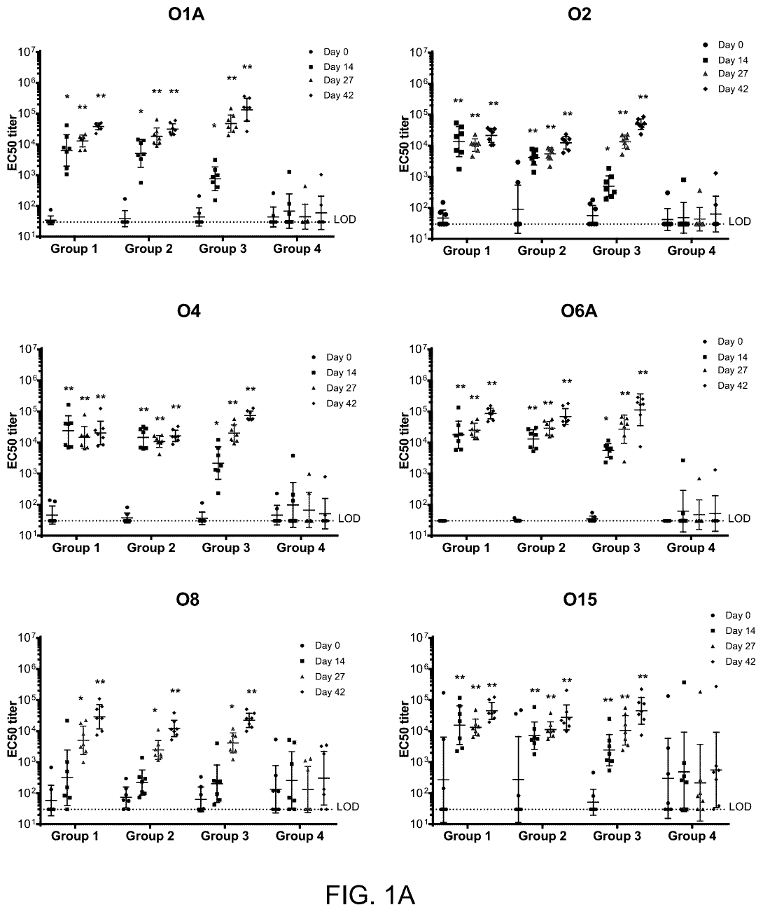

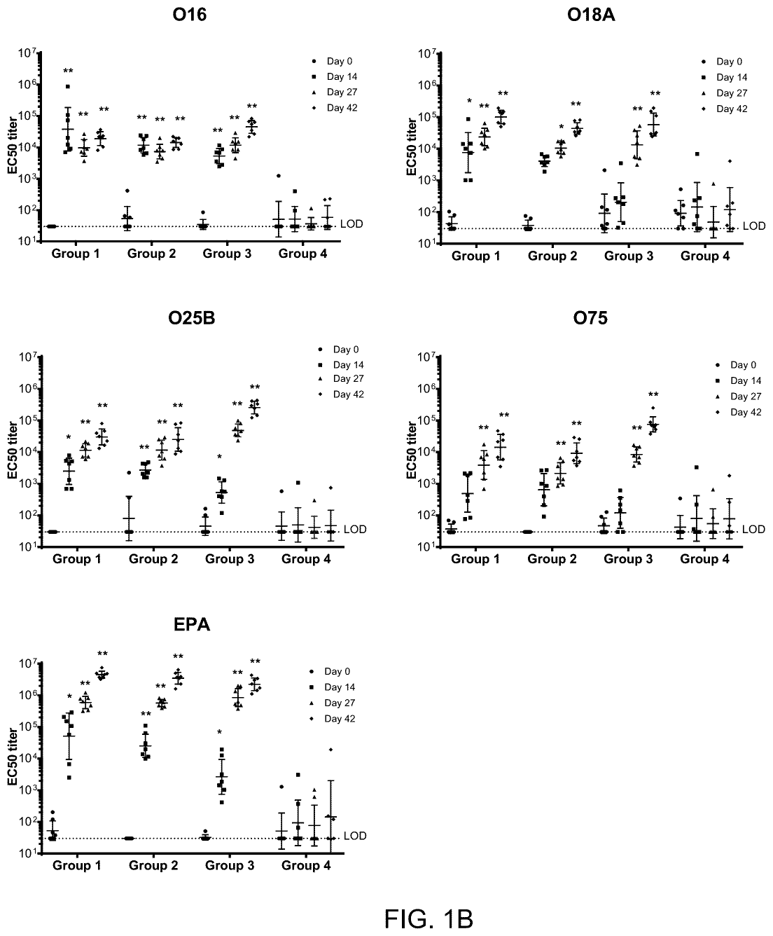

Results are shown in FIG. 1 and summarized in Table 6.

TABLE 6
Summary of E. coli O-antigen specific antibody
responses induced by ExPEC10V in NZW rabbits.

| ExPEC10V | | | | | | | | | | |
|---|---|---|---|---|---|---|---|---|---|---|
| dose | O1A | O2 | O6A | O25B | O4 | O8 | O15^{#} | O16 | O18A | O75 |
| | Antibody responses day 14 post-vaccination | | | | | | | | | |
| High | * | ** | ** | * | ** | ns | ** | ** | * | ns |
| Mid | * | ** | ** | ** | ** | ns | ** | ** | ns | ns |
| Low | * | * | * | * | * | ns | ** | ** | ns | ns |
| | Antibody responses day 27 post-vaccination | | | | | | | | | |
| High | ** | ** | ** | ** | ** | * | ** | ** | ** | ** |
| Mid | ** | ** | ** | ** | ** | * | ** | ** | * | ** |
| Low | ** | ** | ** | ** | ** | * | ** | ** | ** | ** |
| | Antibody responses day 42 post-vaccination | | | | | | | | | |
| High | ** | ** | ** | ** | ** | ** | ** | ** | ** | ** |
| Mid | ** | ** | ** | ** | ** | ** | ** | ** | ** | ** |
| Low | ** | ** | ** | ** | ** | ** | ** | ** | ** | ** |

Serotype-specific antibody responses in which p values were statistically significant are shown by asterisks.
Serotype-specific antibody responses in which p values were not statistically significant are designated as ns.
Wilcoxon Rank Sum test with Bonferroni correction for multiple comparisons.
Comparisons ExPEC10V vaccinated animals (Group 1, 2 and 3) versus saline control (Group 4).
* p ≤ 0.05,
** p ≤ 0.01.
^{#}P values were statistically significant after excluding an outlier animal from the control group (sensitivity analysis).

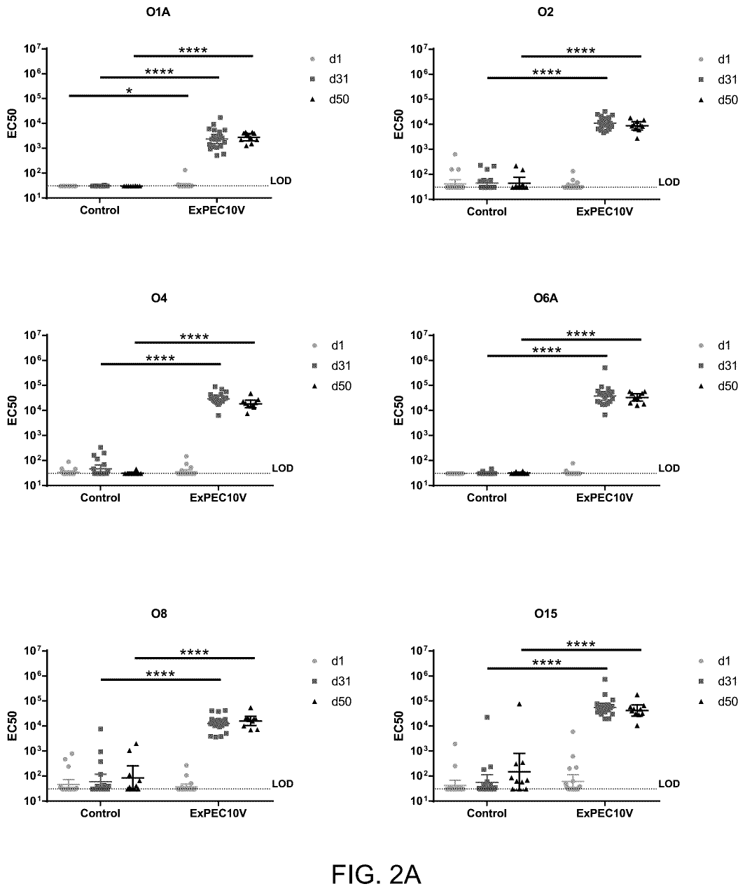

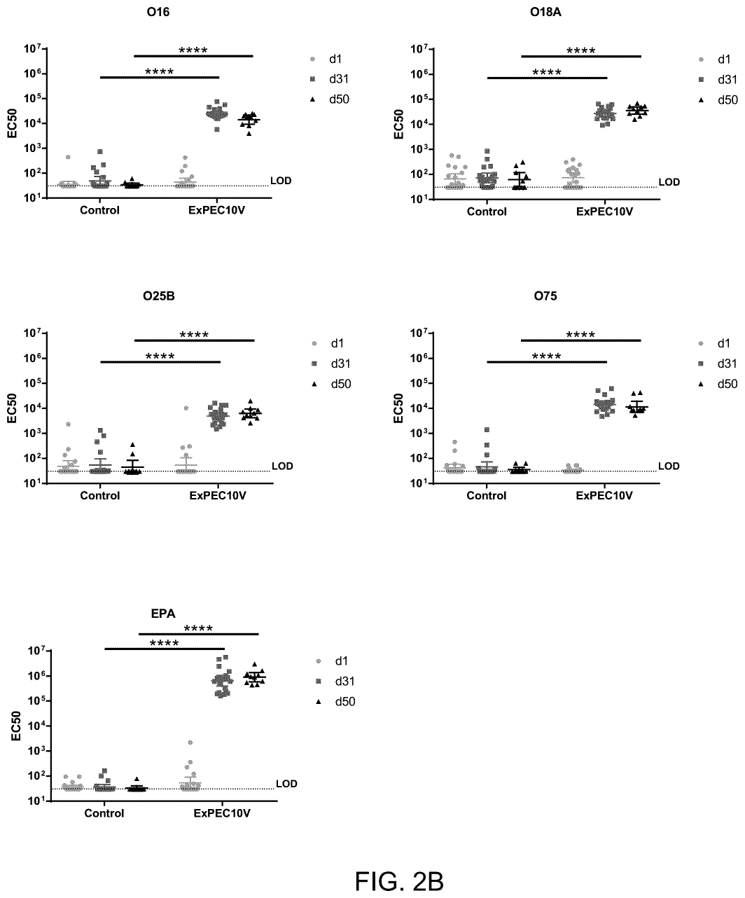

The high dose of ExPEC10V (Group 1) induced significantly higher IgG antibody levels at all time-points investigated (Days 14, 27 and 42 post-immunization) when compared to saline control for O1A, O2, O4, O6A, O16, O18A and O25B (FIG. 2, Table 6). Significantly higher antibody titers induced by O8 and O75 conjugates when compared to saline control were observed at Days 27 and 42 post-immunization (FIG. 1, Table 6).

The medium dose of ExPEC10V (Group 2) and the low dose (Group 3) induced significantly higher antibody levels at all time-points investigated (Days 14, 27 and 42 post-immunization) when compared to saline control for O1A, O2, O4, O6A, O16 and O25B (FIG. 1, Table 6). Significantly higher antibody titers induced by O8, O18A and O75 conjugates when compared to saline control were observed at Days 27 and 42 post-immunization suggesting that the boost dose in rabbits increases the response to these O-serotypes (FIG. 1, Table 6).

For O15 conjugates, sensitivity analysis omitting an outlier animal from the control group showed that all three doses of ExPEC10V vaccine induced a significant increase in antibody responses when compared to saline control at Days 14, 27 and 42 post-immunization (FIG. 1, Table 6).

Antibodies induced by the carrier protein EPA were significantly higher than EPA antibody titers in the saline-treated (control) group for the three doses of ExPEC10V tested (high, medium and low) at all time points investigated (Days 14, 27 and 42) (FIG. 1).

Between dose comparisons (not shown) showed that at Day 14 post-vaccination, the high dose of ExPEC10V induced significantly higher antibody responses when compared to the low dose for most of the conjugates tested (O1A, O2, O4, O6A, O15, O16, O18A and O25B). The medium dose of ExPEC10V also induced significantly higher antibody responses compared to the low dose for O1A, O2, O4, O18A, O25B and O75. For O8 conjugate, all three formulations of ExPEC10V induced similar levels of antibodies at Day 14 post-vaccination.

The low dose of ExPEC10V induced a significant increase in antibody responses at Day 42 post vaccination (after a prime and two boost doses) when compared to the high and medium doses of ExPEC10V for O1A, O2, O4, O16, O25B and O75 conjugates. These findings are in line with other experiences with conjugate vaccines, where for instance no clear relationship between dose and the magnitude of the antibody response to primary vaccination was observed in infants vaccinated with pneumococcal conjugate vaccine (Poolman J T, et al. Expert Rev Vaccines. 2013, 12(12):1379-94).

There were no significant differences between the three doses of ExPEC10V tested at Day 42 post-vaccination for O6A, O8 and O15 conjugates. For the O18A conjugate, the high dose of ExPEC10V induced a significantly higher antibody response when compared to the medium dose at Day 42 post-vaccination.

For the carrier protein (EPA), the high and medium dose of ExPEC10V induced significantly higher antibody responses when compared to the low dose at day 14 post-vaccination. The high dose of the vaccine also induced significantly higher antibody responses when compared to the low dose at day 42 post-vaccination.

In conclusion, the three formulations of ExPEC10V (high, medium and low), administered via intramuscular injection on Days 0, 14, 27 are immunogenic in rabbits.

So far, functional antibodies capable of killing E. coli strains induced by this vaccine in rabbits were shown for serotypes O1A, O2, O4, O6A, O15, O16 and O25B.

In a further experiment, a GMP batch of the ExPEC10V vaccine (see Example 4 above for production) was prepared and injected into NZW rabbits as part of a toxicology study (Table 7). In this study, NZW rabbits (males and females) received 3 intramuscular injections (0.6 mL) of the ExPEC10V vaccine (day 1, 15 and 29) and a control group received 0.9% (w/v) sodium chloride solution (saline). Each dose of the vaccine contained 9.6 g polysaccharide (PS) for serotypes O1A, O2, O4, O6A, O8, O15, O16, O18A and O75 and 19.2 g PS for serotypes O25B, corresponding to 105.6 g total PS (176 g total PS/mL) and 382.8 g of total EPA (638 g EPA/mL). IgG titers against O-antigens and carrier proteins (EPA) were determined from samples collected during the pre-treatment period (day 1) and days 31 and 50 post-immunization.

A significant increase in antibody responses against all O-antigens and the carrier protein EPA were observed at day 31 and 50 post-vaccination in the group that received ExPEC10V when compared to the control group that received only saline (FIG. 2, Table 8). For O1A serotype, a significantly higher antibody response was also observed at day 1 (baseline) when vaccinated animals were compared with the controls. These results suggest that some animals were pre-exposed to E. coli or have antibodies that cross-react with O1A-LPS.

TABLE 7
Experimental groups and ExPEC10V dose used in NZW rabbits.

| | | | | Main | Recovery |
|---|---|---|---|---|---|
| | | | | (day 31) | (day 50) |
| | | | Dosing | (males/ | (males/ |
| Groups | Treatment | Dose | days | females) | females) |
| 1 | control | 0 | 1, 15, 29 | 10 | 10 |
| 2 | ExPEC10V | 105.6 μg PS* | 1, 15, 29 | 10 | 10 |

*Each dose (0.6 mL dosing volume) contains 9.6:9.6:9.6:9.6:9.6:9.6:9.6:9.6:19.2:9.6 μg polysaccharide (PS) for serotypes O1A, O2, O4, O6A, O8, O15, O16, O18A, O25B, O75, respectively (176 μg total PS/mL). Each dose contains 382.8 μg EPA protein (638 μg EPA/mL).

TABLE 8
Immunogenicity of ExPEC10V in NZW rabbits as part to a toxicology study.

| Treatment | Antibody responses day 14 post-vaccination | | | | | | | | | |
|---|---|---|---|---|---|---|---|---|---|---|
| ExPEC10V | O1A | O2 | O6A | O25B | O4 | O8 | O15 | O16 | O18A | O75 |
| Day 31 | **** | **** | **** | **** | **** | **** | **** | **** | **** | **** |
| Day 50 | **** | **** | **** | **** | **** | **** | **** | **** | **** | **** |

Antibody responses induced by ExPEC10V. Serotypes in which a significant increase in antibody responses was observed in the vaccine group compared to control are shown by asterisks. Tobit model with a likelihood ratio test.
**** p ≤ 0.0001.

### Example 7: Phase 1/2a Trial with the ExPEC10V Vaccine in Humans

At present, there is no vaccine available to prevent IED. The serotypes comprising the ExPEC10V vaccine (O1A, O2, O4, O6A, O8, O15, O16, O18A, O25B and O75) were selected to address invasive disease caused by the majority of clinically relevant ExPEC strains that also represent the majority of ExPEC isolates causing antimicrobial resistant IED, including ST131. The selected serotypes are representative for the ten prevalent ExPEC O-serotypes causing bloodstream infections in the older population and responsible for approximately 70% of bloodstream infections caused by ExPEC.

Since the mechanism of action of conjugate vaccines in the prevention of invasive disease is not expected to be affected by antibiotic resistance mechanisms, it is believed that ExPEC10V vaccine provides protection against IED caused by drug-resistant- and drug-susceptible O1A, O2, O4, O6A, O8, O15, O16, O18A, O25B and O75 serotypes.

There is preceding clinical experience with ExPEC4V, an earlier vaccine candidate which comprised a subset of four of the E. coli O-antigen conjugates (O1A, O2, O6A and O25B) also found in ExPEC10V. Based on the results from four completed clinical studies (two phase 1 studies, two phase 2 studies), ExPEC4V was well-tolerated by the study participants and no vaccine-related safety signals were observed at doses up to 16 μg polysaccharide (PS) per serotype (O1A, O2, O6A and O25B). Most adverse events (AEs) were Grade 1 and 2, very few Grade 3 AEs were reported. Late-onset solicited local AEs (AEs which start after Day 5 post-vaccination) were observed mainly with the higher doses of ExPEC4V. In each study, the ExPEC4V vaccine was shown to be immunogenic, demonstrating a dose-dependent vaccine immune response, and O-antigen specific Immunoglobulin G (IgG) titer increases, as measured by enzyme-linked immunosorbent assay (ELISA). Functional activity of the antibodies was demonstrated with an ExPEC4V-optimized opsonophagocytic killing assay (OPKA). Co-analysis of ELISA and OPKA test results showed correlation between the assay responses (Pearson correlation coefficients≥0.61 and >0.48 for Day 30 and Day 360, respectively in a Phase 2 clinical trial [study 4V-BAC2001]), substantiating the use of ELISA as a primary measure of ExPEC4V antibody titers and to predict functional antibody activity. Analysis of the immunogenicity data has demonstrated the durability of the immune response through three years after vaccination with ExPEC4V. It has now also been observed that sera from humans vaccinated with ExPEC4V and that had high titers of serotype-specific opsonophagocytic antibodies, when passively transferred into mice that were subsequently intraperitoneally challenged with E. coli strains of O25B or O2 serotype, were able to mediate protection in vivo, demonstrated by significant reduction in E. coli colony counts (CFU) in blood, spleen and liver (data not shown). Hence, ExPEC4V-specific opsonophagocytic human antibodies mediate bacterial killing in vivo, which is in agreement with other conjugate vaccines in which the proposed mechanism of protection is by induction of opsonophagocytic antibodies that mediate bacterial killing.

ExPEC10V includes a total of ten serotypes and increases coverage from about 50% (ExPEC4V) to approximately 70% of bloodstream infections caused by ExPEC in adults aged 60 years and older. Based on the clinical experience with ExPEC4V, and on the pre-clinical data for ExPEC10V as discussed in the examples above, it was expected that administration of ExPEC10V will induce immune responses to E. coli serotypes O1A, O2, O4, O6A, O8, O15, O16, O18A, O25B and O75 also in humans. Each of the O-antigen polysaccharides (PSs) are separately bioconjugated to the carrier protein, a genetically detoxified form of exotoxin A (EPA) derived from Pseudomonas aeruginosa, and its production has been described above. The O4 PS is the glucosylated form, having the structure of Formula (O4-Glc+) in Table 1.

A randomized, observer-blind, first-in-human phase 1/2a study to evaluate the safety, reactogenicity, and immunogenicity of three different doses of the ExPEC10V vaccine is conducted in humans aged 60 to 85 years in stable health (study 10V-BAC1001). The study design includes 2 cohorts: A total of 824 participants are enrolled in the study with 404 participants (100 participants/ExPEC10V dose) aged ≥60 to ≤85 years in stable health in Cohort 1 and an additional of approximately 420 participants (280 in ExPEC10V group and 140 in placebo group) aged ≥60 years in stable health with a history of UTI in the past 5 years in Cohort 2 (originally it was planned to include 600 participants in Cohort 2, but this number was reduced during the study to approximately 420 with some adaptations to the protocol).

### Objectives and Endpoints

COHORT 1—Phase 1/2a observer-blind period with open-label long-term follow-up period (N=404):

| | |
|---|---|
| Objectives | Endpoints |

Primary

| | |
|---|---|
| To evaluate the safety and reactogenicity of | Solicited local and systemic adverse events |
| different doses of ExPEC10V in participants ≥60 | (AEs) collected for 14 days post-vaccination |
| to ≤85 years of age | (from Day 1 to Day 15) |
| | Unsolicited AEs collected from the |
| | administration of the study vaccine until 29 days |
| | post-vaccination (from Day 1 to Day 30) |
| | Serious adverse events (SAEs) collected from |
| | the administration of the study vaccine until Day |
| | 181 |
| To evaluate the dose-dependent immunogenicity of | Antibody titers for ExPEC10V, as determined |
| ExPEC10V on Day 15 in participants ≥60 to ≤85 | by multiplex electrochemiluminescent (ECL)- |
| years of age | based immunoassay and multiplex |
| | opsonophagocytic assay (MOPA) on Day 15 |

Secondary

| | |
|---|---|
| To evaluate the correlation between multiplex ECL- | Antibody titers for ExPEC10V, as determined |
| based immunoassay (total antibody) and MOPA | by multiplex ECL-based immunoassay and |
| (functional antibody) serum titers on Day 15 | MOPA on Day 15 |
| To evaluate the dose-dependent immunogenicity of | Antibody titers for ExPEC10V, as determined |
| ExPEC10V on Days 30 and 181 in participants ≥60 | by multiplex ECL-based immunoassay and |
| to ≤85 years of age | MOPA on Days 30 and 181 |
| To evaluate, in the long-term follow-up (LTFU) | SAEs related to the study vaccine or study |
| period, the safety of the ExPEC10V dose selected | procedures collected from Day 182 until the end |
| for further clinical development based on the Day | of the study |
| 30 primary analysis in participants ≥60 to ≤85 years | |
| of age | |
| To evaluate, in the LTFU period, the | Antibody titers for ExPEC10V, as determined |
| immunogenicity of the ExPEC10V dose selected | by multiplex ECL-based immunoassay and |
| for further clinical development based on the Day | MOPA at Year 1 (Day 366), Year 2 (Day 731) |
| 30 primary analysis | and Year 3 (Day 1096) |

COHORT 2—Double-blind period with double-blind long-term follow-up period (N=420):

| | |
|---|---|
| Objectives | Endpoints |

Primary

| | |
|---|---|
| To evaluate the safety and reactogenicity of the | Solicited local and systemic AEs collected for |
| selected dose of ExPEC10V in participants ≥60 | 14 days post-vaccination (from Day 1 to |
| years of age with a history of UTI in the past | Day 15) |
| 5 years | Unsolicited AEs collected from the |
| | administration of the study vaccine until 29 days |
| | post-vaccination (from Day 1 to Day 30) |
| | SAEs collected from the administration of the |
| | study vaccine until Day 181 |
| To evaluate the immunogenicity of the selected | Antibody titers for ExPEC10V, as determined |
| dose of ExPEC10V on Day 30 in participants ≥60 | by multiplex ECL-based immunoassay and |
| years of age with a history of UTI in the past 5 years | MOPA on Day 30 |

Secondary

| | |
|---|---|
| To evaluate the correlation between multiplex ECL- | Antibody titers for ExPEC10V, as determined |
| based immunoassay (total antibody) and MOPA | by multiplex ECL-based immunoassay and |
| (functional antibody) serum titers on Day 30 in | MOPA on Day 30 |
| participants ≥60 years of age with a history of UTI | |
| in the past 5 years | |
| To evaluate the immunogenicity of the selected | Antibody titers for ExPEC10V, as determined |
| dose of ExPEC10V on Days 15 and 181 in | by multiplex ECL-based immunoassay on |
| participants ≥60 years of age with a history of UTI | Days 15 and 181 and MOPA on Day 181 |
| in the past 5 years | |
| To evaluate, in the LTFU period, the safety of the | SAEs related to the study vaccine or study |
| selected dose of ExPEC10V in participants ≥60 | procedures collected from Day 182 until the end |
| years of age with a history of UTI in the past 5 | of the study |
| years | |
| To evaluate, in the LTFU period, the | Antibody titers for ExPEC10V, as determined |
| immunogenicity of the selected dose of ExPEC10V | by multiplex ECL-based immunoassay and |
| in participants ≥60 years of age with a history of | MOPA at Year 1 (Day 366), Year 2 (Day 731) |
| UTI in the past 5 years | (not applicable for MOPA), and Year 3 |
| | (Day 1096) |

Exploratory

| | |
|---|---|
| To evaluate the effect of ExPEC10V on the | Metagenomics of stool samples from a selected |
| intestinal (stool) microbiome by metagenomic | subset of participants to evaluate the effect of |
| analyses | ExPEC10V on: |
| | Prevalence of pathogens |
| | (e.g., Clostridium difficile) in the |
| | intestinal flora |
| | Prevalence of ExPEC10V serotypes in the |
| | intestinal flora |

### Overall Design

This is a randomized, multicenter, interventional study including two cohorts.

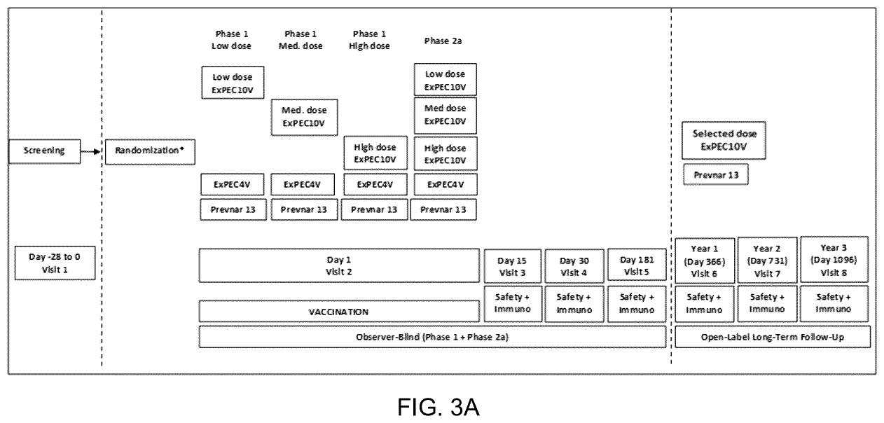

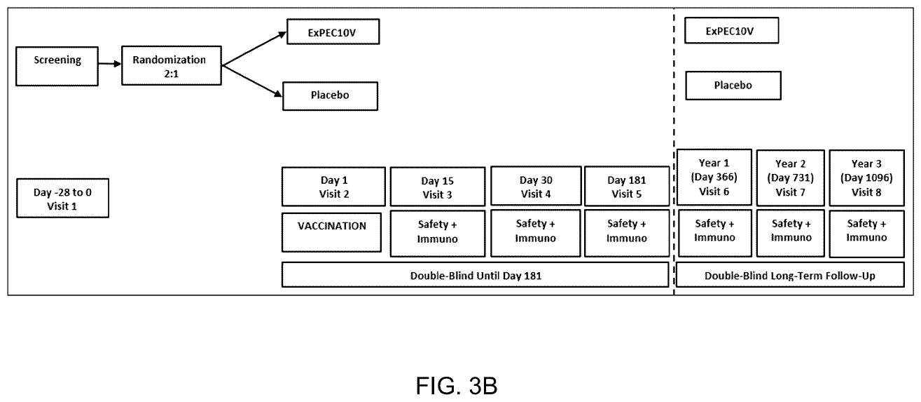

For Cohort 1, the study has an observer-blind, active-controlled design, and a total of 404 adult participants aged ≥60 to ≤85 years in stable health with or without a history of UTI are included. The study design for Cohort 1 is comprised of three periods: a maximum of 28-day screening period, an observer-blinded 181-day follow-up period with vaccination on Day 1 and an open-label LTFU period which lasts from Day 182 until 3 years (Day 1096) post-vaccination (FIG. 3A). Only participants from the ExPEC10V selected dose group (approximately 100 participants) and participants from the Prevnar 13 group progress to the LTFU period. The end of Cohort 1 is the last participant's Year 3 visit (Day 1096).

For Cohort 2, the study has a double-blind, placebo-controlled design, and a total of approximately 420 adult participants aged ≥60 years in stable health with a history of UTI in the past 5 years was included. Enrollment commenced after completion of the Phase 1/2a primary analysis and ExPEC10V dose selection from Cohort 1. The study design for Cohort 2 is comprised of three periods: a maximum 28-day screening period, a double-blind 181-day follow-up period with vaccination on Day 1, and a double-blind LTFU period which lasts from Day 182 until 3 years (Day 1096) post-vaccination (FIG. 3B). All participants in Cohort 2 progress to the LTFU period. The end of study is the last participant's Year 3 visit (Day 1096) in Cohort 2.

### Cohort 1: Phase 1

In Phase 1 of Cohort 1, a total of 84 participants were enrolled in a staggered approach following stepwise dose-escalating procedures with safety evaluations in place before progressing from one step to the next. An internal Data Review Committee (DRC) was commissioned for this study to review the physical examination data (baseline as well as targeted), baseline demographic data and the 14-day post-vaccination safety data (including solicited local and systemic AEs, unsolicited AEs, SAEs, clinical laboratory data and vital signs) of these 84 Phase 1 participants. In this phase of the study, participants were enrolled and randomized in six steps:

Step 1: Four sentinel participants were enrolled and randomized; two participants in the ExPEC10V low dose group (Table 11), and one participant each in the ExPEC4V and Prevnar 13 groups.
Step 2: Twenty-four participants were enrolled and randomized; 18 participants in the ExPEC10V low dose group (Table 11), and three participants each in the ExPEC4V and Prevnar 13 groups.
Step 3: Four sentinel participants were enrolled and randomized; two participants in the ExPEC10V medium dose group (Table 11), and one participant each in the ExPEC4V and Prevnar 13 groups.
Step 4: Twenty-four participants were enrolled and randomized; 18 participants in the ExPEC10V medium dose group (Table 11), and three participants each in the ExPEC4V and Prevnar 13 groups.
Step 5: Four sentinel participants were enrolled and randomized; two participants in the ExPEC10V high dose group (Table 11), and one participant each in the ExPEC4V and Prevnar 13 groups.
Step 6: Twenty-four participants were enrolled and randomized; 18 participants in the ExPEC10V high dose group (Table 11), and three participants each in the ExPEC4V and Prevnar 13 groups.

All participants received a single intramuscular (IM) injection of either ExPEC10V (1 of 3 doses), ExPEC4V or Prevnar 13 on Day 1 per the assigned study vaccination groups. The four sentinel participants at each of Steps 1, 3 and 5 were contacted by telephone 24 hours post-vaccination to collect safety information. The blinded 24-hour post-vaccination safety data in each group of four sentinel participants were reviewed by the principal investigator (PI), study responsible physician (SRP) and sponsor medical lead (SML). Randomization of additional participants for the next step was halted until this Day 2 sentinel safety evaluation was completed.

In the absence of any clinically significant findings, an additional 24 participants (for Steps 2, 4, and 6) were enrolled and randomized to one of three study vaccination groups (Table 11) to receive a single IM injection of either ExPEC10V (1 of 3 doses), ExPEC4V or Prevnar 13 on Day 1.

After vaccination of an additional 24 participants at each dose level (low dose in Step 2, medium dose in Step 4, and high dose in Step 6), 14-day post-vaccination safety data of all 28 (4+24) participants at each dose level was reviewed by the DRC before progressing to the next dose level or Phase 2a.

### Cohort 1: Phase 2a

Based on acceptable safety and reactogenicity (in the absence of any safety concerns or any events meeting a specific study pausing rule) as determined by DRC after the review of 14-day post-vaccination safety data for the initial 84 participants, the remaining 320 participants from Cohort 1 were randomized and dosed in Phase 2a of the study. These additional 320 participants were enrolled and randomized in parallel in a ratio of 2:2:2:1:1 to one of the five study vaccination groups to receive a single IM injection of either ExPEC10V (1 of 3 doses), ExPEC4V or Prevnar 13 on Day 1 (Table 11). In addition to performing the 14-day safety review for the initial 84 participants, the DRC also evaluates safety data of Cohort 1 over the course of the study and reviews any events that meet a specific study vaccination pausing rule or any other safety issue that may arise.

For Cohort 1, the primary analysis occurred when all participants had completed the Day 30 visit (Visit 4) or have discontinued earlier. The final analysis occurs when all participants have completed the Day 181 visit or have discontinued earlier. For participants progressing to the open-label long-term follow-up (LTFU) period (ExPEC10V selected dose group and Prevnar 13 group), yearly follow-up analyses include safety and immunogenicity data (multiplex ECL-based immunoassay and MOPA) collected up to the time of the visit at Year 1 (Day 366), Year 2 (Day 731) and Year 3 (Day 1096) after vaccination.

### Cohort 2

In Cohort 2, the safety, reactogenicity, and immunogenicity of the selected dose of ExPEC10V (based on the primary analysis results of Cohort 1 the high dose was selected) is evaluated in participants aged ≥60 years in stable health with a history of UTI in the past 5 years. For Cohort 2, the study has a double-blind, placebo-controlled design, and a total of approximately 420 participants were enrolled and randomized in parallel in a 2:1 ratio (approximately 280 participants in the ExPEC10V group and approximately 140 in the placebo group).

All participants received a single IM injection of either the selected dose of ExPEC10V or placebo on Day 1 per the assigned study vaccination groups (Table 12).

For Cohort 2, the primary analysis includes safety and immunogenicity data and occurs when all participants have completed the Day 30 visit (Visit 4) or have discontinued earlier. The final analysis occurs when all participants have completed the Day 181 visit or have discontinued earlier. For all participants, yearly follow-up analyses include safety and immunogenicity data (multiplex ECL-based immunoassay and MOPA) collected up to the time of the visit at Year 1 (Day 366), Year 2 (Day 731) (not applicable for MOPA), and Year 3 (Day 1096) after vaccination.

A stool sample analysis is performed in a selected subset of participants to evaluate the effect of ExPEC10V on the prevalence of pathogens (eg, Clostridium difficile) and ExPEC10V serotypes in the intestinal flora using metagenomics.

### Number of Participants

A total of approximately 824 participants was enrolled in the study; 404 participants in Cohort 1 and approximately 420 participants in Cohort 2.

### Intervention Groups: Description of Interventions

ExPEC10V: E. coli bioconjugate vaccine in phosphate buffered solution containing O-antigen PS of ExPEC serotypes O1A, O2, O4, O6A, O8, O15, O16, O18A, O25B and O75 separately bioconjugated to the EPA carrier protein. Single 0.5 mL IM (deltoid) injection of one of the three doses of ExPEC10V on Day 1.

ExPEC4V: E. coli bioconjugate vaccine in saline buffer solution containing O-antigen PS of ExPEC serotypes O1A, O2, O6A, O25B (4:4:4:8 μg PS/ExPEC serotypes) separately bioconjugated to the EPA carrier protein. Single 0.5 mL IM (deltoid) injection of ExPEC4V on Day 1.

Prevnar 13: Sterile suspension of saccharides of the capsular antigens of Streptococcus pneumoniae serotypes 1, 3, 4, 5, 6A, 6B, 7F, 9V, 14, 18C, 19A, 19F, and 23F, individually linked to non-toxic Diphtheria CRM197 protein. Single 0.5 mL IM (deltoid) injection on Day 1, supplied in a single-dose prefilled syringe.

Placebo: normal saline. Single 0.5 mL IM (deltoid) injection of placebo on Day 1.

The ExPEC study intervention materials are described in Table 9.

TABLE 9
BAC1001MV ExPEC Study Vaccines

| | O1A | O2 | O4 | O6A | O8 | O15 | O16 | O18A | O25B | O75 | EPA | PS (Total) |
|---|---|---|---|---|---|---|---|---|---|---|---|---|
| Study Arm | (μg) | (μg) | (μg) | (μg) | (μg) | (μg) | (μg) | (μg) | (μg) | (μg) | (μg) | (μg) |
| Low dose | 4 | 4 | 4 | 4 | 4 | 4 | 4 | 4 | 8 | 4 | 160 | 44 |
| ExPEC10V | | | | | | | | | | | | |
| Medium | 8 | 4 | 4 | 8 | 4 | 4 | 4 | 4 | 16 | 4 | 221 | 60 |
| dose | | | | | | | | | | | | |
| ExPEC10V | | | | | | | | | | | | |
| High dose | 8 | 8 | 8 | 8 | 8 | 8 | 8 | 8 | 16 | 8 | 320 | 88 |
| ExPEC10V | | | | | | | | | | | | |
| ExPEC4V | 4 | 4 | — | 4 | — | — | — | — | 8 | — | 72 | 20 |

EPA = a genetically detoxified form of exotoxin A derived from Pseudomonas aeruginosa;
PS = polysaccharide ExPEC4V consists of the O-antigen polysaccharides (PSs) of the ExPEC serotypes O1A, O2, O6A, and O25B separately bioconjugated to the EPA carrier protein.
ExPEC10V consists of the O-antigen polysaccharides (PSs) of the ExPEC serotypes O1A, O2, O4, O6A, O8, O15, O16, O18A, O25B and O75 separately bioconjugated to the EPA carrier protein.
Dose is based on PS only.
The EPA (μg) are measured values.

ExPEC10V is composed of 10 monovalent drug substances (DSs). For this clinical study, 2 different concentrations (medium and high) of drug product (DP) are produced (Table 10). A third (low) concentration is obtained in the clinic by diluting the high concentration 1:1 with dilution buffer, which is the same as the formulation buffer. Each DP is formulated in Sodium/Potassium phosphate buffer at pH 7.0 (0.02% [w/w] Polysorbate 80, 5% [w/w] sorbitol, 10 mM methionine).

TABLE 10
Composition of ExPEC10V vaccine for phase 1/2a clinical study

| | Amount (μg/mL)^{a} | | |
|---|---|---|---|
| Ingredient | Low | Medium | High |
| Active^{a} | Concentration ^{b} | Concentration | Concentration |
| O-antigen polysaccharide | | | |
| EcoO1A | 8 | 16 | 16 |
| EcoO2 | 8 | 8 | 16 |
| EcoO4 | 8 | 8 | 16 |
| EcoO6A | 8 | 16 | 16 |
| EcoO8 | 8 | 8 | 16 |
| EcoO15 | 8 | 8 | 16 |
| EcoO16 | 8 | 8 | 16 |
| EcoO18A | 8 | 8 | 16 |
| EcoO25B | 16 | 32 | 32 |
| EcoO75 | 8 | 8 | 16 |
| Carrier protein | | | |
| EPA | 320 | 441 | 640 |

Excipients

| | | |
|---|---|---|
| KH₂PO₄ | 6.19 | mM |
| Na₂HPO₄ | 3.81 | mM |
| Sorbitol | 5% | (w/w) |
| Methionine | 10 | mM |
| Polysothate 80 | 0.02% | (w/w) |

EPA = genetically detoxified P. aeruginosa exotoxin A used as carrier protein
^{a}The active ingredient is a biologically synthesized conjugate composed of the PS antigen and a carrier protein (EPA); the dose is calculated on the PS moiety only.
^{b} The “low concentration” is obtained in the clinic by diluting the “high concentration” 1:1 with dilution buffer

### Safety Evaluations

Key safety assessments include solicited local and systemic AEs, unsolicited AEs, SAEs, physical examinations, vital sign measurements, and clinical laboratory tests. IMMUNOGENICITY EVALUATIONS

Key immunogenicity assessments of collected sera include the assessment of ExPEC10V and ExPEC4V serotype-specific total IgG antibody levels elicited by the vaccine as measured by a multiplex ECL-based immunoassay, and ExPEC10V and ExPEC4V serotype-specific functional antibodies as measured by an opsonophagocytic killing assay (OPKA) in multiplex format (MOPA). Immunogenicity assessments of pneumococcal antibody titers elicited by Prevnar 13 are not performed.

The levels of serum antibodies induced by ExPEC10V are measured by a multiplex electrochemiluminescent (ECL)-based immunoassay. This assay combines high binding carbon electrodes in a multi-spot 96-well format microplate that is coated with different E. coli O-LPS antigens or the carrier protein EPA. The levels of antigen-specific antibodies present in serum samples are detected using a secondary antibody (anti-human IgG) labeled with SULFO-TAG. The SULFO-TAG emits light in the presence of electrical stimulation at an intensity that increases proportionally to the amount of bound IgG antibodies. This assay was qualified according to International Conference on Harmonisation (ICH) recommendations.

The levels of functional antibodies induced by ExPEC10V are measured by a multiplex opsonophagocytic assay (MOPA). Briefly, heat-inactivated serum samples are serially diluted and incubated with different E. coli strains that are specifically resistant to different types of antibiotics. After that, human complement and phagocytic cells (HL60) are added to the reaction and, after a second incubation period, an aliquot of the reaction mix is transferred to different PVDF hydrophilic membrane filter plates containing media supplemented with specific antibiotic that selectively allow growth of a strain that is resistant to that particular antibiotic. After overnight growth, the colony forming units (CFUs) are counted to determine the number of surviving bacteria. This assay was qualified according to ICH recommendations.

For ExPEC10V serotype antibodies as measured by multiplex ECL-based immunoassay and MOPA, and EPA as measured by multiplex ECL-based immunoassay only, the following measures of immunogenicity are evaluated and tabulated by the study vaccination groups, for all immunogenicity time points:
proportion of participants with a ≥2-fold and >4-fold increase in serum antibody titers from Day 1 (pre-vaccination)
geometric mean titer (GMT)
GMR: fold change from baseline, calculated from the post-baseline/baseline value.
   For the LTFU period, descriptive summaries of immunogenicity are provided for each serotype.

Dose selection for later phases considers the totality of the evidence available at the time of the primary analysis of Cohort 1 (Day 30 results).

TABLE 11
Cohort 1: Vaccination Schedule

| | | Phase 1 | | | | | | | |
|---|---|---|---|---|---|---|---|---|---|
| | | | | Step 3 | Step 4 | | | Phase 2a | |
| | | Step 1 | Step 2 | Sentinel | Additional | Step 5 | Step 6 | Step 7 | |
| Study | | Sentinel | Additional | participants | participants | Sentinel | Additional | Additional | |
| Vaccination | Vaccination | participants | participants | (Medium | (Medium | participants | participants | Phase 2a | |
| Group | on Day 1 | (Low dose) | (Low dose) | dose) | dose) | (High dose) | (High dose) | Participants | Total |
| G1 | Low dose | 2 | 18 | | | | | 80 | 100 |
| | ExPEC10V* | | | | | | | | |
| G2 | Medium dose | | | 2 | 18 | | | 80 | 100 |
| | ExPEC10V* | | | | | | | | |
| G3 | High dose | | | | | 2 | 18 | 80 | 100 |
| | ExPEC10V* | | | | | | | | |
| G4 | ExPEC4V** | 1 | 3 | 1 | 3 | 1 | 3 | 40 | 52 |
| G5 | Prevnar 13*** | 1 | 3 | 1 | 3 | 1 | 3 | 40 | 52 |
| Total | | 4 | 24 | 4 | 24 | 4 | 24 | 320 | 404 |

*ExPEC10V consists of the O-antigen polysaccharides (PSs) of the ExPEC serotypes O1A, O2, O4, O6A, O8, O15, O16, O18A, O25B and O75 separately bioconjugated to the carrier protein, a genetically detoxified form of exotoxin A (EPA) derived from Pseudomonas aeruginosa.
**ExPEC4V consists of the O-antigen polysaccharides (PSs) of the ExPEC serotypes O1A, O2, O6A, and O25B separately bioconjugated to the carrier protein, a genetically detoxified form of exotoxin A (EPA) derived from Pseudomonas aeruginosa.
***Prevnar 13, Pneumococcal 13-valent conjugate vaccine (Diphtheria CRM197 protein) is a sterile suspension of saccharides of the capsular antigens of Streptococcus pneumoniae serotypes 1, 3, 4, 5, 6A, 6B, 7F, 9V, 14, 18C, 19A, 19F, and 23F, individually linked to non-toxic Diphtheria CRM197 protein.

TABLE 12
Cohort 2: Vaccination Schedule

| Study Vaccination | Vaccination on | |
|---|---|---|
| Group | Day 1 | Total |
| G6 | ExPEC10V^{a} | 280 |
| G7 | Placebo | 140 |
| Total | | 420 |

aExPEC10V consists of the O-antigen polysaccharides (PSs) of the ExPEC serotypes O1A, O2, O4, O6A, O8, O15, O16, O18A, O25B, and O75 separately bioconjugated to the carrier protein, a genetically detoxified form of exotoxin A (EPA) derived from Pseudomonas aeruginosa.

The randomization ratio for the participants enrolled in Cohort 2 of the study is 2:1 (ExPEC10V:Placebo). The ExPEC10V dose used in Cohort 2 was based on the primary analysis (Day 30) results of Cohort 1, and this is the high dose that was used in Cohort 1.

### Status

Enrollment and vaccination of Cohort 1 of the study described above was completed. No major safety issues were identified, and the ExPEC10V vaccine has an acceptable safety profile.

The analysis of the immunogenicity of the Cohort 1 clinical samples was performed and results are described below.

The Cohort 2 vaccinations were started using the high dose for ExPEC10V, and this part of the study is ongoing.

### Cohort 1 Safety and Immunogenicity Results

These results presented here refer to the BAC1001 clinical study (cohort 1). There were 416 participants in the full analysis set. Each participant was randomized to either 1 of 3 single intra-muscular administered doses of ExPEC10V or to 1 of 2 active control doses of ExPEC4V or Prevnar; 104 were vaccinated with the ExPEC10V low dose, 102 with the ExPEC10V medium dose, 104 with the ExPEC10V high dose, 52 with the ExPEC4V (4:4:4:8 μg O1A, O2, O6A and O25B polysaccharide/dose), and 54 with Prevnar. A total of 413 participants completed the Day 30 visit. There were 392 (94.2%) participants included in the Day 15 per protocol immunogenicity analysis.

### Safety

Overall, all ExPEC10V doses were well tolerated. There was a trend for increased reactogenicity with high doses, with the ExPEC10V high dose reactogenicity lower or comparable to Prevnar, except for erythema, swelling and nausea. In contrast to Prevnar, more late onset local reactogenicity was observed in ExPEC10V groups with injection site erythema and swelling reported as late onset events in >90% of participants experiencing these local events. However, the reactogenicity profile of ExPEC10V is acceptable when compared to other licensed vaccines used in older adult populations.

### Immunogenicity

Vaccine-induced immune responses were assessed at baseline (day 1 pre-vaccination) and at day 15 post-vaccination using a multiplex electrochemiluminescent (ECL)-based immunoassay that measures the levels of serotype-specific serum antibodies (total immunoglobulin G [IgG]) and a multiplex opsonophagocytic killing assay (MOPA) that measures antibody mediated bacterial opsonophagocytic killing.

### Levels of Antigen-Specific Serum Antibodies (ECL)

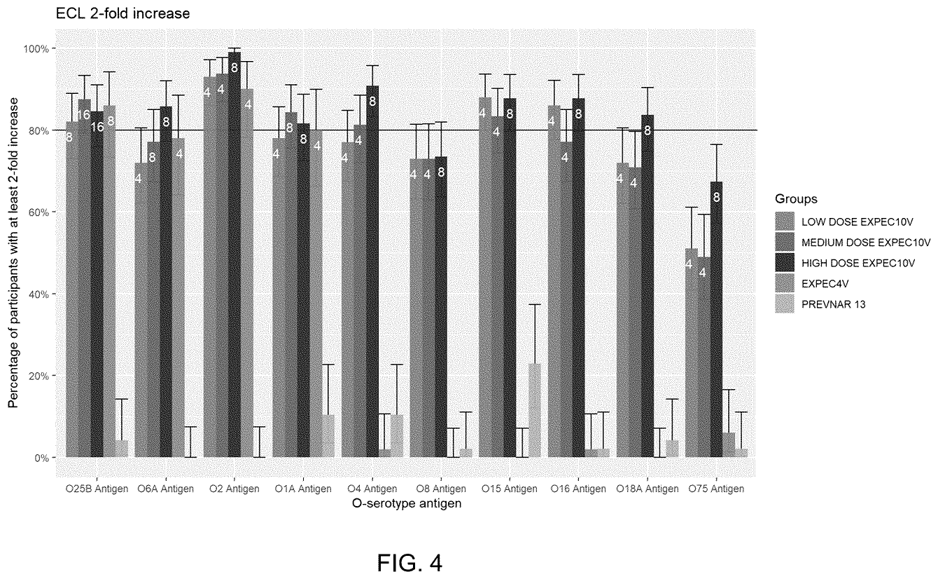

Levels of O-antigen-specific serum antibodies (total IgG) increased significantly on Day 15 post-vaccination for all ExPEC10V doses tested and all vaccine-related serotypes. It was observed that at least 80% of the subjects that received the high dose of the ExPEC10V vaccine showed a two-fold or greater increase in O-antigen-specific antibody titers by Day 15 post-vaccination for 8 out of 10 serotypes that include O25B, O6A, O2, O1A, O4, O15, O16 and O18A. For the ExPEC10V medium dose group, at least 80% of subjects show a two-fold or greater increase in O-antigen specific antibody titers by Day 15 post-vaccination, for 5 out of 10 serotypes that include O25B, O2, O1A, O4 and O15; and for the ExPEC10V low dose group for 4 out of 10 serotypes that include O25B, O2, O15 and O16 (FIG. 4, Table 13). Minimal changes in the GMT from day 1 to day 15 were observed in the group of subjects that received Prevnar or ExPEC4V (for the non-ExPEC4V serotypes) (FIG. 4, Table 13).

For the ECL assay, all subjects were analyzed and included in the all immunogenicity subset. Subjects with major violations according to protocol exclusion criteria were excluded from the per protocol analyses. The geometric mean titer (GMT), GMT FI (geometric mean titer fold-increase) and percentage of participants with at least 2- and 4-fold increases from baseline in the ECL-based immunoassay are summarized in Table 13 below.

TABLE 13
ECL data BAC1001 cohort 1

| | | | | | | |
|---|---|---|---|---|---|---|
| Endpoint | Time | ExPEC10V | ExPEC10V | ExPEC10V | | |
| (95% CI) | point | Low Dose | Medium Dose | High Dose | ExPEC4V | Prevnar |

Serotype O1A

| | | | | | | |
|---|---|---|---|---|---|---|
| GMTs | Day 1 | 1226879.8 | 1193952.5 | 1202227.3 | 1072873.1 | 1310616.9 |
| | | (1030417.6; | (978207.8; | (999407.5; | (798770.8; | (1010870.8; |
| | | 1460800.0) | 1457279.9) | 1446207.5) | 1441035.1) | 1699244.5) |
| GMTs | Day 15 | 5328463.0 | 6351780.5 | 6379531.1 | 6539744.0 | 1650517.3 |
| | | (4620273.9; | (5647000.4; | (5682021.0; | (5315259.2; | (1228541.5; |
| | | 6145202.3) | 7144521.5) | 7162665.7) | 8046315.4) | 2217432.2) |
| GM FI¹ | Day 15 vs | 4.35 | 5.26 | 5.19 | 5.99 | 1.27 |
| | Day 1 | (3.646; | (4.361; | (4.310; | (4.459; | (1.094; |
| | | 5.198) | 6.345) | 6.256) | 8.043) | 1.485) |
| % 2-fold² | Day 15 vs | 78.0% | 84.4% | 81.6% | 80.0% | 10.4% |
| | Day 1 | (68.61%; | (75.54%; | (72.53%; | (66.28%; | (3.47%; |
| | | 85.67%) | 90.98%) | 88.74%) | 89.97%) | 22.66%) |
| % 4-fold² | Day 15 vs | 56.0% | 61.5% | 64.3% | 66.0% | 6.3% |
| | Day 1 | (45.72%; | (50.97%; | (53.97%; | (51.23%; | (1.31%; |
| | | 65.92%) | 71.22%) | 73.71%) | 78.79%) | 17.20%) |

Serotype O2

| | | | | | | |
|---|---|---|---|---|---|---|
| GMTs | Day 1 | 510014.8 | 465679.8 | 534252.7 | 477186.7 | 467863.4 |
| | | (424544.1; | (393668.5; | (456163.2; | (365264.4; | (354874.1; |
| | | 612692.6) | 550863.6) | 625710.3) | 623403.6) | 616827.7) |
| GMTs | Day 15 | 4846804.8 | 4779406.7 | 6505196.8 | 6174400.7 | 431229.4 |
| | | (4088473.9; | (4015130.7; | (5820337.7; | (4983834.3; | (334539.7; |
| | | 5745791.1) | 5689162.0) | 7270640.9) | 7649376.5) | 555864.6) |
| GM FI¹ | Day 15 vs | 9.48 | 10.09 | 12.46 | 12.70 | 1.01 |
| | Day 1 | (7.955; | (8.389; | (10.686; | (9.383; | (0.952; |
| | | 11.306) | 12.132) | 14.516) | 17.196) | 1.067) |
| % 2-fold² | Day 15 vs | 93.0% | 93.8% | 99.0% | 90.0% | 0.0% |
| | Day 1 | (86.11%; | (86.89%; | (94.45%; | (78.19%; | (0.00%; |
| | | 97.14%) | 97.67%) | 99.97%) | 96.67%) | 7.40%) |
| % 4-fold² | Day 15 vs | 83.0% | 84.4% | 92.9% | 82.0% | 0.0% |
| | Day 1 | (74.18%; | (75.54%; | (85.84%; | (68.56%; | (0.00%; |
| | | 89.77%) | 90.98%) | 97.08%) | 91.42%) | 7.40%) |

Serotype O4

| | | | | | | |
|---|---|---|---|---|---|---|
| GMTs | Day 1 | 461135.6 | 492816.2 | 449573.2 | 462018.9 | 429691.9 |
| | | (394351.8; | (420242.9; | (396266.8; | (378746.2; | (352681.9; |
| | | 539229.2) | 577922.5) | 510050.4) | 563600.3) | 523517.5) |
| GMTs | Day 15 | 2876992.7 | 2776953.7 | 4135064.0 | 479974.7 | 556386.8 |
| | | (2312781.5; | (2271070.8; | (3466612.3; | (389335.0; | (418115.4; |
| | | 3578845.2) | 3395522.4) | 4932410.4) | 591716.0) | 740384.7) |
| GM FI¹ | Day 15 vs | 6.26 | 5.52 | 9.15 | 1.06 | 1.29 |
| | Day 1 | (5.080; | (4.543; | (7.568; | (1.006; | (1.085; |
| | | 7.716) | 6.717) | 11.063) | 1.124) | 1.524) |
| % 2-fold² | Day 15 vs | 77.0% | 81.3% | 90.8% | 2.0% | 10.4% |
| | Day 1 | (67.51%; | (72.00%; | (83.28%; | (0.05%; | (3.47%; |
| | | 84.83%) | 88.49%) | 95.71%) | 10.65%) | 22.66%) |
| % 4-fold² | Day 15 vs | 64.0% | 63.5% | 79.6% | 0.0% | 4.2% |
| | Day 1 | (53.79%; | (53.09%; | (70.26%; | (0.00%; | (0.51%; |
| | | 73.36%) | 73.13%) | 87.07%) | 7.11%) | 14.25%) |

Serotype O6A

| | | | | | | |
|---|---|---|---|---|---|---|
| GMTs | Day 1 | 1220500.4 | 1143877.4 | 1127737.5 | 1178385.6 | 943745.8 |
| | | (1060415.0; | (965179.1; | (987356.6; | (934832.4; | (776652.7; |
| | | 1404753.0) | 1355660.9) | 1288077.7) | 1485392.0) | 1146788.3) |
| GMTs | Day 15 | 4314596.8 | 5145946.5 | 5839456.8 | 4930272.7 | 1012716.0 |
| | | (3748795.9; | (4369752.3; | (5126873.3; | (3947184.1; | (821125.5; |
| | | 4965793.3) | 6060015.3) | 6651082.1) | 6158210.2) | 1249009.7) |
| GM FI¹ | Day 15 vs | 3.55 | 4.45 | 5.06 | 4.38 | 1.04 |
| | Day 1 | (3.033; | (3.691; | (4.355; | (3.407; | (0.986; |
| | | 4.145) | 5.370) | 5.869) | 5.618) | 1.088) |
| % 2-fold² | Day 15 vs | 72.0% | 77.1% | 85.7% | 78.0% | 0.0% |
| | Day 1 | (62.13%; | (67.39%; | (77.19%; | (64.04%; | (0.00%; |
| | | 80.52%) | 85.05%) | 91.96%) | 88.47%) | 7.40%) |
| % 4-fold² | Day 15 vs | 43.0% | 52.1% | 65.3% | 52.0% | 0.0% |
| | Day 1 | (33.14%; | (41.64%; | (55.02%; | (37.42%; | (0.00%; |
| | | 53.29%) | 62.39%) | 74.64%) | 66.34%) | 7.40%) |

Serotype O8

| | | | | | | |
|---|---|---|---|---|---|---|
| GMTs | Day 1 | 1534906.4 | 1732564.1 | 1535536.4 | 1537793.8 | 1339640.3 |
| | | (1315284.3; | (1488898.1; | (1319371.4; | (1250768.0; | (1061371.9; |
| | | 1791200.5) | 2016107.4) | 1787117.9) | 1890686.4) | 1690864.7) |
| GMTs | Day 15 | 5052165.0 | 5600945.1 | 6178848.4 | 1709293.2 | 1374269.5 |
| | | (4405019.0; | (4902756.3; | (5511236.6; | (1392733.5; | (1075143.6; |
| | | 5794383.9) | 6398561.1) | 6927332.3) | 2097805.0) | 1756617.9) |
| GM FI¹ | Day 15 vs | 3.35 | 3.26 | 3.94 | 1.07 | 1.05 |
| | Day 1 | (2.896; | (2.777; | (3.381; | (1.034; | (0.994; |
| | | 3.871) | 3.832) | 4.584) | 1.116) | 1.111) |
| % 2-fold² | Day 15 vs | 73.0% | 72.9% | 73.5% | 0.0% | 2.1% |
| | Day 1 | (63.20%; | (62.89%; | (63.59%; | (0.00%; | (0.05%; |
| | | 81.39%) | 81.48%) | 81.88%) | 7.11%) | 11.07%) |
| % 4-fold² | Day 15 vs | 42.0% | 39.6% | 52.0% | 0.0% | 0.0% |
| | Day 1 | (32.20%; | (29.75%; | (41.71%; | (0.00%; | (0.00%; |
| | | 52.29%) | 50.08%) | 62.24%) | 7.11%) | 7.40%) |

Serotype O15

| | | | | | | |
|---|---|---|---|---|---|---|
| GMTs | Day 1 | 871316.5 | 865326.5 | 869436.4 | 847989.9 | 839725.1 |
| | | (728072.4; | (733883.5; | (742857.9; | (659076.0; | (668057.1; |
| | | 1042743.2) | 1020311.8) | 1017583.0) | 1091053.0) | 1055505.9) |
| GMTs | Day 15 | 5145327.6 | 4658173.1 | 5502286.9 | 859569.9 | 1208928.4 |
| | | (4381397.1; | (3991308.2; | (4828131.0; | (666001.9; | (882681.8; |
| | | 6042455.2) | 5436457.2) | 6270575.8) | 1109397.0) | 1655758.5) |
| GM FI¹ | Day 15 vs | 5.93 | 5.37 | 6.24 | 1.01 | 1.48 |
| | Day 1 | (4.945; | (4.496; | (5.276; | (0.973; | (1.227; |
| | | 7.100) | 6.408) | 7.391) | 1.055) | 1.797) |
| % 2-fold² | Day 15 vs | 88.0% | 83.3% | 87.8% | 0.0% | 22.9% |
| | Day 1 | (79.98%; | (74.35%; | (79.59%; | (0.00%; | (12.03%; |
| | | 93.64%) | 90.16%) | 93.51%) | 7.11%) | 37.31%) |
| % 4-fold² | Day 15 vs | 61.0% | 63.5% | 68.4% | 0.0% | 10.4% |
| | Day 1 | (50.73%; | (53.09%; | (58.20%; | (0.00%; | (3.47%; |
| | | 70.60%) | 73.13%) | 77.39%) | 7.11%) | 22.66%) |

Serotype O16

| | | | | | | |
|---|---|---|---|---|---|---|
| GMTs | Day 1 | 860263.7 | 853674.6 | 803233.9 | 675579.3 | 706903.3 |
| | | (725964.5; | (740655.4; | (691840.6; | (572649.9; | (568193.4; |
| | | 1019407.5) | 983939.8) | 932562.7) | 797009.5) | 879475.6) |
| GMTs | Day 15 | 4265641.1 | 3973364.4 | 5630267.2 | 750895.2 | 744487.3 |
| | | (3633602.0; | (3373538.4; | (4906785.0; | (615148.2; | (588274.4; |
| | | 5007618.8) | 4679841.3) | 6460423.5) | 916598.0) | 942181.5) |
| GM FI¹ | Day 15 vs | 5.00 | 4.56 | 6.93 | 1.11 | 1.08 |
| | Day 1 | (4.262; | (3.794; | (5.817; | (0.991; | (1.002; |
| | | 5.870) | 5.489) | 8.249) | 1.247) | 1.154) |
| % 2-fold² | Day 15 vs | 86.0% | 77.1% | 87.8% | 2.0% | 2.1% |
| | Day 1 | (77.63%; | (67.39%; | (79.59%; | (0.05%; | (0.05%; |
| | | 92.13%) | 85.05%) | 93.51%) | 10.65%) | 11.07%) |
| % 4-fold² | Day 15 vs | 63.0% | 57.3% | 72.4% | 2.0% | 0.0% |
| | Day 1 | (52.76%; | (46.78%; | (62.50%; | (0.05%; | (0.00%; |
| | | 72.44%) | 67.34%) | 80.99%) | 10.65%) | 7.40%) |

Serotype O18A

| | | | | | | |
|---|---|---|---|---|---|---|
| GMTs | Day 1 | 934049.6 | 963410.7 | 923828.6 | 948044.5 | 979084.2 |
| | | (819694.2; | (838467.8; | (816988.8; | (795966.9; | (788765.0; |
| | | 1064358.5) | 1106971.8) | 1044640.1) | 1129178.2) | 1215325.0) |
| GMTs | Day 15 | 3517970.2 | 3585716.9 | 4539608.6 | 972430.2 | 1087640.8 |
| | | (2948912.8; | (3003087.3; | (3893880.0; | (803752.4; | (828956.9; |
| | | 4196839.6) | 4281382.5) | 5292419.4) | 1176507.2) | 1427049.5) |
| GM FI¹ | Day 15 vs | 3.79 | 3.62 | 4.85 | 1.04 | 1.14 |
| | Day 1 | (3.219; | (3.020; | (4.130; | (0.996; | (1.029; |
| | | 4.470) | 4.341) | 5.702) | 1.086) | 1.256) |
| % 2-fold² | Day 15 vs | 72.0% | 70.8% | 83.7% | 0.0% | 4.2% |
| | Day 1 | (62.13%; | (60.67%; | (74.84%; | (0.00%; | (0.51%; |
| | | 80.52%) | 79.67%) | 90.37%) | 7.11%) | 14.25%) |
| % 4-fold² | Day 15 vs | 49.0% | 41.7% | 61.2% | 0.0% | 2.1% |
| | Day 1 | (38.86%; | (31.68%; | (50.85%; | (0.00%; | (0.05%; |
| | | 59.20%) | 52.18%) | 70.90%) | 7.11%) | 11.07%) |

Serotype O25B

| | | | | | | |
|---|---|---|---|---|---|---|
| GMTs | Day 1 | 246235.1 | 240586.0 | 243305.0 | 189072.4 | 267582.0 |
| | | (205202.9; | (200618.4; | (204854.9; | (149829.5; | (207517.9; |
| | | 295472.1) | 288515.9) | 288972.0) | 238593.9) | 345031.0) |
| GMTs | Day 15 | 1413686.1 | 2275453.1 | 2047161.5 | 2026104.2 | 270695.3 |
| | | (1100919.7; | (1768282.1; | (1617468.8; | (1428854.6; | (202827.4; |
| | | 1815308.2) | 2928088.7) | 2591005.3) | 2872999.1) | 361272.4) |
| GM FI¹ | Day 15 vs | 5.78 | 9.61 | 8.19 | 10.36 | 1.03 |
| | Day 1 | (4.732; | (7.509; | (6.474; | (7.430; | (0.929; |
| | | 7.054) | 12.298) | 10.363) | 14.445) | 1.136) |
| % 2-fold² | Day 15 vs | 82.0% | 87.5% | 83.7% | 86.0% | 4.2% |
| | Day 1 | (73.05%; | (79.18%; | (74.84%; | (73.26%; | (0.51%; |
| | | 88.97%) | 93.37%) | 90.37%) | 94.18%) | 14.25%) |
| % 4-fold² | Day 15 vs | 58.0% | 76.0% | 69.4% | 76.0% | 2.1% |
| | Day 1 | (47.71%; | (66.25%; | (59.26%; | (61.83%; | (0.05%; |
| | | 67.80%) | 84.17%) | 78.30%) | 86.94%) | 11.07%) |

Serotype O75

| | | | | | | |
|---|---|---|---|---|---|---|
| GMTs | Day 1 | 1258387.9 | 1329257.4 | 1284187.5 | 1107038.5 | 1373337.6 |
| | | (1054541.9; | (1126527.2; | (1101597.2; | (902508.7; | (1061821.6; |
| | | 1501637.9) | 1568471.0) | 1497042.2) | 1357919.5) | 1776245.9) |
| GMTs | Day 15 | 2952821.0 | 3102330.4 | 3923938.0 | 1317142.3 | 1368220.0 |
| | | (2468576.2; | (2620338.5; | (3410142.4; | (1048701.5; | (1042053.0; |
| | | 3532057.1) | 3672981.2) | 4515145.6) | 1654297.0) | 1796478.7) |
| GM FI¹ | Day 15 vs | 2.34 | 2.36 | 3.01 | 1.14 | 1.04 |
| | Day 1 | (2.019; | (2.039; | (2.579; | (1.023; | (0.942; |
| | | 2.715) | 2.734) | 3.510) | 1.260) | 1.139) |
| % 2-fold² | Day 15 vs | 51.0% | 49.0% | 67.3% | 6.0% | 2.1% |
| | Day 1 | (40.80%; | (38.61%; | (57.13%; | (1.25%; | (0.05%; |
| | | 61.14%) | 59.37%) | 76.48%) | 16.55%) | 11.07%) |
| % 4-fold² | Day 15 vs | 21.0% | 25.0% | 32.7% | 4.0% | 2.1% |
| | Day 1 | (13.49%; | (16.72%; | (23.52%; | (0.49%; | (0.05%; |
| | | 30.29%) | 34.88%) | 42.87%) | 13.71%) | 11.07%) |

Cohort 1: output for the primary analysis
¹= GM of FI from baseline (Day 1)
²= percentage of participants with fold increase from baseline (Day 1)
CI = Confidence interval;
GM = Geometric mean,
GMT = Geometric mean titer;
FI = Fold increase
95% CI for GMT and GM FI is based on the t-distribution
95% CI for Percentage of Participants With 2- and 4-Fold Increase is obtained using Clopper-Pearson method.

It can be seen from the data (FIG. 4) that the total IgG response to O75 polysaccharide antigen is lower compared to the other antigens administered in the same amount (e.g. lower percentage of subjects with at least a two-fold increase on day 15 vs day 1), which was a surprising result. The data also show that an increase of the amount of O75 polysaccharide antigen in the composition (i.e. administration of 8 μg in the high dose vs 4 μg in the low and medium doses) increased the induced immune response to O75 polysaccharide antigen.

### Serum Antibodies Functionality (MOPA)

Partial data were included in the cohort 1 MOPA analysis. Table 14 shows the number of participants included in the MOPA GMT analysis at Day 15. Although partial data was used for the evaluation of the functionality of the antibody response induced by ExPEC10V, the conclusions of the analysis did not change when the complete data set was available.

TABLE 14
Number of participants included in the MOPA GMT analysis

| Sero- | Low | Medium | High | | | | |
|---|---|---|---|---|---|---|---|
| type | Dose | Dose | Dose | ExPEC4V | | Prevnar | |
| Expect- | 100 | 96 | 98 | 50 | | 48 | |
| ed | | | | | | | |
| number | | | | | | | |
| O25B | 93 (93%) | 94 (98%) | 93 (95%) | 48 | (96%) | 46 | (96%) |
| O6A | 98 (98%) | 94 (98%) | 97 (99%) | 48 | (96%) | 48 | (100%) |
| O2 | 59 (59%) | 54 (56%) | 44 (45%) | 24 | (48%) | 45 | (94%) |
| O1A | 92 (92%) | 94 (98%) | 88 (90%) | 40 | (80%) | 46 | (96%) |
| O4 | 98 (98%) | 93 (97%) | 97 (99%) | 50 | (100%) | 47 | (98%) |
| O8 | 99 (98%) | 94 (98%) | 96 (98%) | 50 | (100%) | 46 | (96%) |
| O15 | 85 (85%) | 90 (94%) | 92 (94%) | 49 | (98%) | 45 | (94%) |
| O16 | 100 | 94 (98%) | 96 (98%) | 50 | (100%) | 48 | (100%) |
| | (100%) | | | | | | |
| O18A | 89 (89%) | 87 (91%) | 92 (94%) | 45 | (90%) | 43 | (90%) |
| O75 | 95 (95%) | 92 (96%) | 89 (91%) | 47 | (94%) | 46 | (96%) |

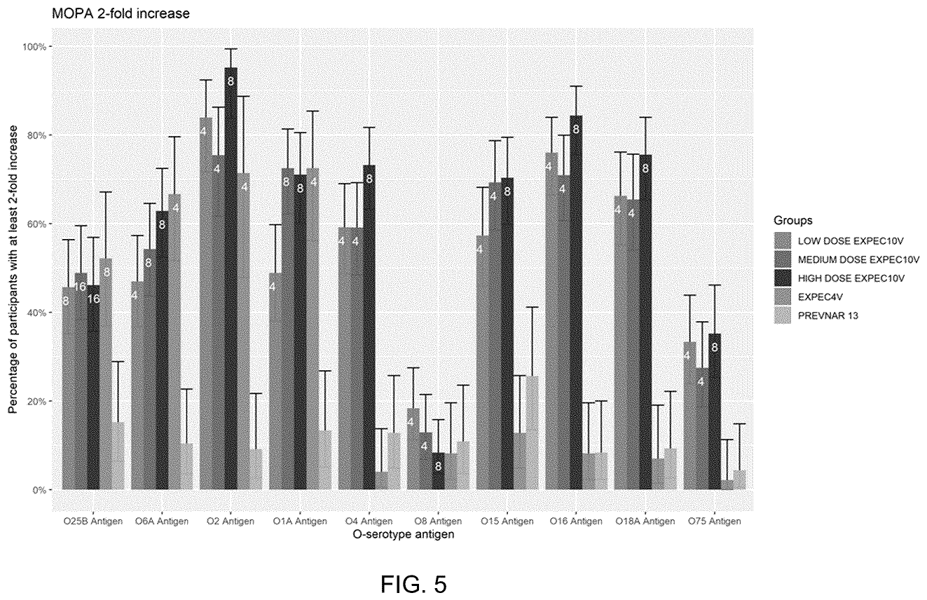

Levels of opsonophagocytic antibodies measured by MOPA were generally consistent with the levels of O-antigen-specific antibodies (total IgG) measured by ECL-based immunoassay. Increased levels of functional antibodies were observed on Day 15 for nine out of the ten ExPEC10V serotypes at all dose groups tested (FIG. 5).

In contrast to the other ExPEC10V serotypes, for serotype O8 the qualified MOPA assay was unable to show a vaccine-mediated increase in functional antibodies on day 15 following ExPEC10V vaccination on Day 1 (FIG. 5), although an increase in serotype O8 total IgG titers was observed (FIG. 4). Studies were initiated to distinguish whether the qualified assay was unable to detect functional O8 antibodies. Subsequent experiments with a modified MOPA assay and Day 1 and Day 15 sera successfully showed Day 15 functional antibody increases of 2-fold or more (data not shown), demonstrating that the ExPEC10V vaccine did actually induce functional antibodies also for serotype O8. For clinical development, the modified O8 MOPA would require re-qualification of the assay and retesting of clinical samples, which takes substantial time, and it was decided to initially proceed with development of a vaccine composition that does not include the O8 polysaccharide antigen. However, it is clear from the subsequent experiments that the O8 bioconjugate in the 10-valent ExPEC vaccine composition does induce functional antibodies against the E. coli O8 serotype, and hence such bioconjugate can likely be added successfully to the nine-valent vaccine compositions described in the example below.

The highest levels of opsonophagocytic antibodies were observed for the serotype O2; 95% of the subjects that were vaccinated with the high dose of that vaccine showed a 2-fold or greater increase in anti-O2 opsonic antibody titers at day 15 post-vaccination. Excluding O8 serotype, for which limited levels of functional antibodies were detected as discussed above, the lowest levels of opsonophagocytic antibodies were observed for the serotype O75; 36% of the subjects that were vaccinated with the high dose of the vaccine showed a 2-fold or greater increase in anti-O75 opsonic antibody titers at day 15 post-vaccination. The MOPA data for the O75 serotype are thus consistent with the ECL data for this serotype described above, corroborating the relatively lower immunogenicity of conjugates of E. coli O75 antigen polysaccharide as compared to several other conjugates in the composition at the same concentration, e.g. of E. coli O6, O1, or O2 conjugates. In addition, minimal changes in the GMT from day 1 to day 15 were observed in the group of subjects that received Prevnar or ExPEC4V (for the non-ExPEC4V serotypes) (FIG. 5), which is in line with expectations for serotypes not present in the respective vaccine compositions.

The geometric mean titers (GMT), geometric mean fold increase (GMR FI) and percentage of participants with at least 2- and 4-fold increase from baseline in the MOPA are summarized in Table 15 for each of the 10 serotypes.

TABLE 15
MOPA data BAC1001 cohort 1

| | | | | | | |
|---|---|---|---|---|---|---|
| Endpoint | Time | ExPEC10V | ExPEC10V | ExPEC10V | | |
| (95% CI) | point | Low Dose | Medium Dose | High Dose | ExPEC4V | Prevnar |

Serotype O1A

| | | | | | | |
|---|---|---|---|---|---|---|
| GMTs | Day 1 | 221.5 | 154.8 | 171.3 | 255.1 | 201.1 |
| | | (179.7; | (125.5; | (136.7; | (179.2; | (154.1; |
| | | 272.9) | 190.8) | 214.8) | 363.2) | 262.5) |
| GMTs | Day 15 | 606.2 | 748.5 | 995.9 | 974.5 | 226.3 |
| | | (469.6; | (584.4; | (738.1; | (620.4; | (167.0; |
| | | 782.5) | 958.8) | 1343.7) | 1530.7) | 306.7) |
| GM FI¹ | Day 15 vs | 2.67 | 4.41 | 5.63 | 4.29 | 1.12 |
| | Day 1 | (2.068; | (3.473; | (4.102; | (2.868; | (0.895; |
| | | 3.455) | 5.589) | 7.739) | 6.406) | 1.413) |
| % 2-fold² | Day 15 vs | 48.9% | 72.5% | 72.0% | 72.5% | 13.3% |
| | Day 1 | (38.05%; | (62.17%; | (60.94%; | (56.11%; | (5.05%; |
| | | 59.75%) | 81.37%) | 81.32%) | 85.40%) | 26.79%) |
| % 4-fold² | Day 15 vs | 28.4% | 48.4% | 51.2% | 47.5% | 2.2% |
| | Day 1 | (19.30%; | (37.74%; | (39.92%; | (31.51%; | (0.06%; |
| | | 39.02%) | 59.07%) | 62.42%) | 63.87%) | 11.77%) |

Serotype O2

| | | | | | | |
|---|---|---|---|---|---|---|
| GMTs | Day 1 | 370.7 | 305.7 | 385.2 | 403.5 | 370.0 |
| | | (298.5; | (239.5; | (312.2; | (287.3; | (272.5; |
| | | 460.4) | 390.2) | 475.3) | 566.6) | 502.5) |
| GMTs | Day 15 | 1951.9 | 1535.4 | 3455.8 | 2810.9 | 349.6 |
| | | (1436.3; | (1116.0; | (2424.9; | (1502.2; | (261.6; |
| | | 2652.5) | 2112.5) | 4925.1) | 5259.6) | 467.2) |
| GM FI¹ | Day 15 vs | 5.77 | 6.38 | 11.18 | 8.28 | 1.05 |
| | Day 1 | (4.283; | (4.434; | (7.676; | (3.432; | (0.901; |
| | | 7.778) | 9.172) | 16.272) | 19.966) | 1.212) |
| % 2-fold² | Day 15 vs | 83.9% | 75.5% | 95.2% | 71.4% | 9.1% |
| | Day 1 | (71.67%; | (61.72%; | (83.84%; | (47.82%; | (2.53%; |
| | | 92.38%) | 86.24%) | 99.42%) | 88.72%) | 21.67%) |
| % 4-fold² | Day 15 vs | 62.5% | 60.4% | 76.2% | 57.1% | 0.0% |
| | Day 1 | (48.55%; | (46.00%; | (60.55%; | (34.02%; | (0.00%; |
| | | 75.08%) | 73.55%) | 87.95%) | 78.18%) | 8.04%) |

Serotype O4

| | | | | | | |
|---|---|---|---|---|---|---|
| GMTs | Day 1 | 116.4 | 82.1 | 89.8 | 101.6 | 93.8 |
| | | (96.5; | (66.8; | (77.2; | (73.2; | (72.9; |
| | | 140.4) | 100.9) | 104.4) | 141.0) | 120.8) |
| GMTs | Day 15 | 359.7 | 296.9 | 458.3 | 104.1 | 98.4 |
| | | (269.3; | (222.6; | (350.9; | (75.4; | (75.1; |
| | | 480.5) | 395.8) | 598.5) | 143.8) | 129.0) |
| GM FI¹ | Day 15 vs | 3.22 | 3.20 | 4.96 | 1.00 | 1.09 |
| | Day 1 | (2.526; | (2.557; | (3.915; | (0.874; | (0.916; |
| | | 4.114) | 3.993) | 6.283) | 1.140) | 1.294) |
| % 2-fold² | Day 15 vs | 59.2% | 59.1% | 73.2% | 4.0% | 12.8% |
| | Day 1 | (48.79%; | (48.46%; | (63.24%; | (0.49%; | (4.83%; |
| | | 69.01%) | 69.23%) | 81.68%) | 13.71%) | 25.74%) |
| % 4-fold² | Day 15 vs | 35.7% | 37.6% | 55.7% | 2.0% | 4.3% |
| | Day 1 | (26.29%; | (27.79%; | (45.23%; | (0.05%; | (0.52%; |
| | | 46.03%) | 48.28%) | 65.76%) | 10.65%) | 14.54%) |

Serotype O6A

| | | | | | | |
|---|---|---|---|---|---|---|
| GMTs | Day 1 | 632.8 | 559.0 | 628.0 | 495.8 | 623.6 |
| | | (489.5; | (431.0; | (505.0; | (338.8; | (458.6; |
| | | 818.0) | 724.9) | 781.0) | 725.6) | 848.1) |
| GMTs | Day 15 | 1454.0 | 2146.6 | 2227.9 | 1984.9 | 695.0 |
| | | (1109.3; | (1651.0; | (1685.7; | (1335.8; | (489.5; |
| | | 1905.8) | 2791.0) | 2944.7) | 2949.3) | 986.7) |
| GM FI¹ | Day 15 vs | 2.24 | 3.74 | 3.59 | 4.11 | 1.20 |
| | Day 1 | (1.833; | (2.764; | (2.798; | (2.773; | (1.023; |
| | | 2.733) | 5.069) | 4.597) | 6.084) | 1.397) |
| % 2-fold² | Day 15 vs | 46.9% | 54.3% | 62.9% | 66.7% | 10.4% |
| | Day 1 | (36.78%; | (43.66%; | (52.48%; | (51.59%; | (3.47%; |
| | | 57.29%) | 64.58%) | 72.48%) | 79.60%) | 22.66%) |
| % 4-fold² | Day 15 vs | 23.5% | 41.5% | 41.2% | 39.6% | 2.1% |
| | Day 1 | (15.50%; | (31.41%; | (31.33%; | (25.77%; | (0.05%; |
| | | 33.11%) | 52.12%) | 51.69%) | 54.73%) | 11.07%) |

Serotype O8

| | | | | | | |
|---|---|---|---|---|---|---|
| GMTs | Day 1 | 967.6 | 699.2 | 896.3 | 710.0 | 891.8 |
| | | (764.2; | (538.5; | (714.1; | (535.1; | (684.4; |
| | | 1225.1) | 907.9) | 1125.1) | 941.9) | 1162.1) |
| GMTs | Day 15 | 1205.4 | 859.9 | 937.3 | 758.4 | 930.0 |
| | | (945.7; | (657.8; | (748.9; | (558.5; | (698.3; |
| | | 1536.5) | 1124.2) | 1173.0) | 1029.9) | 1238.6) |
| GM FI¹ | Day 15 vs | 1.22 | 1.22 | 1.09 | 1.04 | 1.03 |
| | Day 1 | (1.055; | (1.102; | (0.961; | (0.845; | (0.846; |
| | | 1.422) | 1.346) | 1.226) | 1.271) | 1.242) |
| % 2-fold² | Day 15 vs | 18.4% | 12.9% | 8.3% | 8.2% | 10.9% |
| | Day 1 | (11.26%; | (6.85%; | (3.67%; | (2.27%; | (3.62%; |
| | | 27.47%) | 21.45%) | 15.76%) | 19.60%) | 23.57%) |
| % 4-fold² | Day 15 vs | 4.1% | 3.2% | 3.1% | 4.1% | 2.2% |
| | Day 1 | (1.12%; | (0.67%; | (0.65%; | (0.50%; | (0.06%; |
| | | 10.12%) | 9.14%) | 8.86%) | 13.98%) | 11.53%) |

Serotype O15

| | | | | | | |
|---|---|---|---|---|---|---|
| GMTs | Day 1 | 579.8 | 440.5 | 517.6 | 720.2 | 528.0 |
| | | (443.0; | (322.1; | (394.8; | (472.1; | (370.3; |
| | | 758.7) | 602.5) | 678.6) | 1098.6) | 752.9) |
| GMTs | Day 15 | 2509.1 | 2747.1 | 3355.7 | 641.2 | 777.6 |
| | | (1742.6; | (1887.8; | (2272.3; | (432.6; | (498.7; |
| | | 3612.8) | 3997.5) | 4955.5) | 950.4) | 1212.6) |
| GM FI¹ | Day 15 vs | 4.30 | 6.40 | 6.70 | 0.96 | 1.54 |
| | Day 1 | (2.989; | (4.355; | (4.508; | (0.697; | (1.077; |
| | | 6.199) | 9.408) | 9.948) | 1.331) | 2.206) |
| % 2-fold² | Day 15 vs | 57.3% | 69.3% | 70.3% | 13.0% | 25.6% |
| | Day 1 | (45.91%; | (58.58%; | (59.84%; | (4.94%; | (13.52%; |
| | | 68.18%) | 78.71%) | 79.45%) | 26.26%) | 41.17%) |
| % 4-fold² | Day 15 vs | 39.0% | 54.5% | 56.0% | 8.7% | 14.0% |
| | Day 1 | (28.44%; | (43.58%; | (45.25%; | (2.42%; | (5.30%; |
| | | 50.43%) | 65.20%) | 66.44%) | 20.79%) | 27.93%) |

Serotype O16

| | | | | | | |
|---|---|---|---|---|---|---|
| GMTs | Day 1 | 215.7 | 186.7 | 173.0 | 227.6 | 189.2 |
| | | (170.2; | (149.3; | (139.4; | (164.3; | (146.1; |
| | | 273.4) | 233.6) | 214.6) | 315.4) | 244.9) |
| GMTs | Day 15 | 1367.2 | 942.6 | 1591.7 | 217.7 | 175.7 |
| | | (1005.3; | (697.1; | (1190.4; | (150.4; | (130.1; |
| | | 1859.5) | 1274.4) | 2128.3) | 315.2) | 237.3) |
| GM FI¹ | Day 15 vs | 5.90 | 5.00 | 9.12 | 0.92 | 0.99 |
| | Day 1 | (4.428; | (3.655; | (6.807; | (0.778; | (0.832; |
| | | 7.852) | 6.845) | 12.223) | 1.092) | 1.175) |
| % 2-fold² | Day 15 vs | 76.0% | 71.0% | 84.4% | 8.2% | 8.3% |
| | Day 1 | (66.43%; | (60.64%; | (75.54%; | (2.27%; | (2.32%; |
| | | 83.98%) | 79.92%) | 90.98%) | 19.60%) | 19.98%) |
| % 4-fold² | Day 15 vs | 55.0% | 50.5% | 75.0% | 2.0% | 4.2% |
| | Day 1 | (44.73%; | (39.97%; | (65.12%; | (0.05%; | (0.51%; |
| | | 64.97%) | 61.07%) | 83.28%) | 10.85%) | 14.25%) |

Serotype O18A

| | | | | | | |
|---|---|---|---|---|---|---|
| GMTs | Day 1 | 65.6 | 51.8 | 52.7 | 63.6 | 52.8 |
| | | (51.3; | (39.0; | (42.1; | (42.2; | (39.4; |
| | | 83.9) | 68.9) | 66.1) | 95.8) | 70.8) |
| GMTs | Day 15 | 251.0 | 248.8 | 310.0 | 68.3 | 62.4 |
| | | (180.9; | (184.9; | (221.7; | (45.2; | (40.1; |
| | | 348.1) | 334.7) | 433.4) | 103.4) | 97.1) |
| GM FI¹ | Day 15 vs | 3.91 | 4.52 | 5.65 | 1.10 | 1.24 |
| | Day 1 | (2.946; | (3.259; | (4.190; | (0.930; | (0.961; |
| | | 5.193) | 6.256) | 7.624) | 1.309) | 1.609) |
| % 2-fold² | Day 15 vs | 66.3% | 65.4% | 76.4% | 7.1% | 9.5% |
| | Day 1 | (55.28%; | (54.04%; | (66.22%; | (1.50%; | (2.66%; |
| | | 76.12%) | 75.66%) | 84.76%) | 19.48%) | 22.62%) |
| % 4-fold² | Day 15 vs | 44.2% | 46.9% | 55.1% | 4.8% | 7.1% |
| | Day 1 | (33.48%; | (35.73%; | (44.14%; | (0.58%; | (1.50%; |
| | | 55.30%) | 58.33%) | 65.62%) | 16.16%) | 19.48%) |

Serotype O25B

| | | | | | | |
|---|---|---|---|---|---|---|
| GMTs | Day 1 | 157.9 | 145.0 | 149.8 | 147.1 | 162.2 |
| | | (121.4; | (109.6; | (117.6; | (105.5; | (115.8; |
| | | 205.3) | 191.8) | 191.0) | 204.9) | 227.2) |
| GMTs | Day 15 | 399.1 | 497.4 | 387.9 | 460.1 | 245.0 |
| | | (303.8; | (375.7; | (295.5; | (312.1; | (154.5; |
| | | 524.2) | 658.4) | 509.2) | 678.3) | 388.6) |
| GM FI¹ | Day 15 vs | 2.23 | 2.70 | 2.28 | 2.83 | 1.42 |
| | Day 1 | (1.792; | (2.117; | (1.833; | (1.932; | (1.004; |
| | | 2.768) | 3.453) | 2.826) | 4.143) | 2.015) |
| % 2-fold² | Day 15 vs | 45.7% | 48.9% | 46.2% | 52.2% | 15.2% |
| | Day 1 | (35.22%; | (38.34%; | (35.64%; | (36.95%; | (6.34%; |
| | | 56.37%) | 59.56%) | 56.92%) | 67.11%) | 28.87%) |
| % 4-fold² | Day 15 vs | 23.9% | 31.5% | 26.4% | 39.1% | 4.3% |
| | Day 1 | (15.63%; | (22.23%; | (17.69%; | (25.09%; | (0.53%; |
| | | 33.94%) | 42.04%) | 36.65%) | 54.63%) | 14.84%) |

Serotype O75

| | | | | | | |
|---|---|---|---|---|---|---|
| GMTs | Day 1 | <LLOQ | <LLOQ | <LLOQ | <LLOQ | <LLOQ |
| | | (<LLOQ; | (<LLOQ; | (<LLOQ; | (<LLOQ; | (<LLOQ; |
| | | 39.3) | <LLOQ) | <LLOQ) | 37.2) | 40.1) |
| GMTs | Day 15 | 77.1 | 55.6 | 71.0 | <LLOQ | <LLOQ |
| | | (60.6; | (44.6; | (53.9; | (<LLOQ; | (<LLOQ; |
| | | 98.1) | 69.4) | 93.7) | 37.2) | 44.0) |
| GM FI¹ | Day 15 vs | 1.88 | 1.65 | 2.10 | 1.04 | 1.04 |
| | Day 1 | (1.548; | (1.404; | (1.700; | (0.944; | (0.945; |
| | | 2.275) | 1.941) | 2.596) | 1.147) | 1.156) |
| % 2-fold² | Day 15 vs | 33.7% | 27.5% | 36.0% | 2.3% | 4.4% |
| | Day 1 | (24.17%; | (18.63%; | (25.97%; | (0.06%; | (0.54%; |
| | | 44.30%) | 37.83%) | 47.12%) | 12.02%) | 15.15%) |
| % 4-fold² | Day 15 vs | 15.2% | 15.4% | 22.1% | 2.3% | 0.0% |
| | Day 1 | (8.58%; | (8.67%; | (13.86%; | (0.06%; | (0.00%; |
| | | 24.21%) | 24.46%) | 32.33%) | 12.02%) | 7.87%) |

Cohort 1: output for the primary analysis
¹= GM of FI from baseline (Day 1)
²= percentage of participants with fold increase from baseline (Day 1)
CI = Confidence interval;
GM = Geometric mean,
GMT = Geometric mean titer;
FI = Fold increase
95% CI for GMT and GM FI is based on the t-distribution
95% CI for Percentage of Participants With 2- and 4-Fold Increase is obtained using Clopper-Pearson method.

### Dose Selection

The immunogenicity dose selection algorithm based on the mean of the log 10 fold increase from baseline to Day 15 of each dose group identified the ExPEC10V high dose for cohort 2 of this trial considering the two analysis sets (PPI and FAS) and the two assays (ECL and MOPA).

For serotypes O2, O4, O15, O16 and O18A, the high dose group had higher GMT, GMR and percentage of participants with 2-fold and 4-fold increases at Day 15 compared to the other 2 ExPEC10V doses in both assays. For serotype O25B, the medium dose group generally had higher GMT, GMR and percentage of participants with 2-fold and 4-fold increases among the 3 ExPEC10V dose groups; for this serotype the O-antigen dose was the same (16 μg) for both the medium and the high ExPEC10V dose groups. For both serotype O1A and O6A, the results were mixed between the high and the medium dose. In serotype O75, there was a measurable functional antibody response for the ExPEC10V doses. Measurable functional antibody responses were not detected in the qualified assay for serotype O8, as discussed above. In the non-ExPEC4V serotypes, low functional antibody responses were observed in the 2 active controls; the antibody response came from pre-existing antibodies rather than from vaccine-induced response. Based on these results, the high dose of ExPEC10V was selected for the cohort 2 of BAC1001 clinical trial.

### CONCLUSION

ExPEC10V vaccine induced robust antibody responses, an increase in O-antigen specific serum antibodies were observed for all vaccine-related serotypes at day 15 post-vaccination when compared to baseline titers (day 1). ExPEC10V-induced functional antibodies that mediated E. coli opsonophagocytic killing were detected in a qualified MOPA for all vaccine-related serotypes, except for the serotype O8. For serotype O8, subsequent studies with a modified MOPA did measure a functional antibody response. Notably, the O75 response was weaker at 4 μg and 8 μg doses than for the other serotypes at the same dose, which was a surprising and unpredictable finding from this study. To improve the immune response to E. coli O75 serotype generated by multivalent glycoconjugate compositions, in one aspect the instant invention increases the dose of serotype O75 antigen polysaccharide, e.g., to about 1.2 to 8 times the dose of some of the other serotypes, e.g. about 1.5 to 4 times, e.g. about 1.5 to 2.5 times, e.g. about 2 times the dose of some of the other serotypes such as O6 or O1, e.g. to about 16 μg, which is higher than for instance three of the most relevant and prevalent serotypes O1A, O2, and O6A, and similar to serotype O25B antigen polysaccharide. It is noted that in earlier experiments (see e.g. above in this example as well as examples 5 and 6, i.e. before the clinical data with the ExPEC10V vaccine disclosed herein were available) the dose of O75 antigen polysaccharide was at best as high as the doses of O1A, O2 and O6A antigen polysaccharides, or actually in certain tested groups even two times lower than O1A and O6A antigen polysaccharides, and even 2 to 4 times lower than O25B antigen polysaccharide. In contrast, in certain aspects of the instant invention the dose of O75 is thus actually increased compared to the dose of O1, O2, and/or O6.

### Cohort 2 Safety and Immunogenicity Results

In Cohort 2 of this study, a total of approximately 420 participants aged ≥60 year in stable health with a history of UTI in the past 5 year were enrolled and randomized in parallel in a 2:1 ratio (the set on which a full results analysis was performed included 278 participants in the ExPEC10V (high dose) group and 138 in the placebo group).

### Safety

Overall, the ExPEC10V vaccine was well tolerated. The reactogenicity profile was generally comparable to that observed with medium and high doses of ExPEC10V in Cohort 1. The most frequent local solicited AE was pain/tenderness and the most frequently reported systemic AEs were myalgia, headache, and fatigue. Injection site erythema and swelling were the most common late-onset events (time to first onset>5 days after vaccination). The reactogenicity profile of ExPEC10V is acceptable when compared to other licensed vaccines used in older adult populations. Immunogenicity

Vaccine-induced antibody responses were assessed at baseline (day 1 pre-vaccination) and at day 15 and day 30 post-vaccination using a multiplex ECL-based immunoassay that measures the levels of serotype-specific serum antibodies (total IgG).

### Levels of Antigen-Specific Serum Antibodies (ECL, Day 1, Day 15 and Day 30)

The ExPEC10V vaccine was immunogenic for all serotypes based on the increasing value of the GMT, GMT FI and percentage of participants with at least 2-fold increase in titer from baseline from Day 1 to Day 15. There were minimal changes in all three measures from Day 15 to Day 30.

The highest antibody responses were observed for the serotypes O2, O4, O15 and O16 with at least 80% of the participants vaccinated with ExPEC10V showing a two-fold or greater increase in antibody responses by Day 15 and Day 30 post-vaccination. For the serotypes O1A, O6A and O25B, at least 70% of participants show a two-fold or greater increase in antibody response and for the serotypes O8 and O18A at least 60% of the participants show a two-fold or greater increase in antibody response. Lower antibody responses were observed for the serotype O75, nevertheless, a two-fold or greater increase in antigen-specific antibody response was observed in at least 50% of the participants. Minimal changes in the GMT from day 1 to day 15 and day 30 were observed in the group of subjects that received placebo (Table 16).

The geometric mean titer (GMT), GMT FI (geometric mean titer fold-increase) and percentage of participants with at least 2- and 4-fold increases from baseline in the ECL-based immunoassay are summarized in Table 16 below.

TABLE 16
ECL data BAC1001 cohort 2 (day 1, day 15 and day 30)

| | | | |
|---|---|---|---|
| Endpoint | Time | | |
| (95% CI) | point | ExPEC10V | Placebo |

Serotype O1A

| | | | | | |
|---|---|---|---|---|---|
| GMTs | Day 1 | 1466300.6 | (1309861.4; 1641423.6) | 1714154.2 | (1463104.9; 2008280.3) |
| GMTs | Day 15 | 6474621.3 | (5996773.2; 6990546.4) | 1696032.9 | (1445852.1; 1989503.3) |
| GMTs | Day 30 | 6363928.5 | (5918207.5; 6843218.4) | 1773323.4 | (1515713.5; 2074716.7) |
| GM FI¹ | Day 15 vs Day 1 | 4.38 | (3.908; 4.902) | 0.99 | (0.966; 1.023) |
| GM FI¹ | Day 30 vs Day 1 | 4.35 | (3.915; 4.843) | 1.02 | (0.954; 1.094) |
| % 2-fold² | Day 15 vs Day 1 | 75.9% | (70.13%; 81.03%) | 0.0% | (0.00%; 2.84%) |
| % 2-fold² | Day 30 vs Day 1 | 77.6% | (72.04%; 82.46%) | 1.5% | (0.19%; 5.45%) |
| % 4-fold² | Day 15 vs Day 1 | 54.5% | (48.19%; 60.79%) | 0.0% | (0.00%; 2.84%) |
| % 4-fold² | Day 30 vs Day 1 | 54.4% | (48.14%; 60.50%) | 0.8% | (0.02%; 4.21%) |

Serotype O2

| | | | | | |
|---|---|---|---|---|---|
| GMTs | Day 1 | 739313.9 | (659799.6; 828410.8) | 768325.6 | (648274.3; 910608.6) |
| GMTs | Day 15 | 6001149.0 | (5522390.3; 6521413.2) | 759366.1 | (637561.8; 904440.8) |
| GMTs | Day 30 | 5995886.9 | (5530975.6; 6499876.9) | 747487.9 | (627602.8; 890273.4) |
| GM FI¹ | Day 15 vs Day 1 | 8.02 | (7.105; 9.043) | 1.00 | (0.933; 1.061) |
| GM FI¹ | Day 30 vs Day 1 | 8.16 | (7.244; 9.182) | 0.98 | (0.910; 1.054) |
| % 2-fold² | Day 15 vs Day 1 | 88.9% | (84.40%; 92.52%) | 0.8% | (0.02%; 4.28%) |
| % 2-fold² | Day 30 vs Day 1 | 89.4% | (84.98%; 92.81%) | 1.5% | (0.19%; 5.45%) |
| % 4-fold² | Day 15 vs Day 1 | 78.7% | (73.09%; 83.54%) | 0.8% | (0.02%; 4.28%) |
| % 4-fold² | Day 30 vs Day 1 | 77.6% | (72.04%; 82.46%) | 1.5% | (0.19%; 5.45%) |

Serotype O4

| | | | | | |
|---|---|---|---|---|---|
| GMTs | Day 1 | 678578.0 | (616370.1; 747064.4) | 744119.6 | (646038.0; 857092.0) |
| GMTs | Day 15 | 3746755.5 | (3336959.5; 4206876.5) | 760274.2 | (652833.0; 885397.7) |
| GMTs | Day 30 | 3570627.3 | (3188275.4; 3998832.6) | 806768.3 | (691960.6; 940624.6) |
| GM FI¹ | Day 15 vs Day 1 | 5.60 | (4.965; 6.309) | 1.03 | (0.992; 1.067) |
| GM FI¹ | Day 30 vs Day 1 | 5.31 | (4.743; 5.955) | 1.09 | (1.014; 1.166) |
| % 2-fold² | Day 15 vs Day 1 | 82.6% | (77.37%; 87.07%) | 1.6% | (0.19%; 5.53%) |
| % 2-fold² | Day 30 vs Day 1 | 81.7% | (76.54%; 86.23%) | 2.3% | (0.48%; 6.60%) |
| % 4-fold² | Day 15 vs Day 1 | 62.1% | (55.77%; 68.06%) | 0.8% | (0.02%; 4.28%) |
| % 4-fold² | Day 30 vs Day 1 | 58.9% | (52.73%; 64.94%) | 1.5% | (0.19%; 5.45%) |

Serotype O6A

| | | | | | |
|---|---|---|---|---|---|
| GMTs | Day 1 | 1491643.9 | (1353036.8; 1644450.1) | 1836907.8 | (1582827.3; 2131774.0) |
| GMTs | Day 15 | 5624986.5 | (5131740.4; 6165641.6) | 1881936.5 | (1613534.0; 2194986.4) |
| GMTs | Day 30 | 5516879.9 | (5049584.3; 6027419.7) | 1934121.0 | (1657028.0; 2257550.3) |
| GM FI¹ | Day 15 vs Day 1 | 3.92 | (3.548; 4.339) | 1.03 | (0.991; 1.073) |
| GM FI¹ | Day 30 vs Day 1 | 3.64 | (3.295; 4.026) | 1.06 | (0.991; 1.135) |
| % 2-fold² | Day 15 vs Day 1 | 74.7% | (68.88%; 79.94%) | 0.8% | (0.02%; 4.28%) |
| % 2-fold² | Day 30 vs Day 1 | 73.0% | (67.21%; 78.27%) | 2.3% | (0.48%; 6.60%) |
| % 4-fold² | Day 15 vs Day 1 | 50.2% | (43.87%; 56.52%) | 0.0% | (0.00%; 2.84%) |
| % 4-fold² | Day 30 vs Day 1 | 44.5% | (38.38%; 50.72%) | 0.8% | (0.02%; 4.21%) |

Serotype O8

| | | | | | |
|---|---|---|---|---|---|
| GMTs | Day 1 | 2145103.2 | (1937289.9; 2375208.6) | 2335274.5 | (2038224.0; 2675617.0) |
| GMTs | Day 15 | 6281926.9 | (5810107.0; 6792061.6) | 2380959.0 | (2063441.9; 2747334.9) |
| GMTs | Day 30 | 6286969.0 | (5832409.6; 6776955.4) | 2303456.2 | (2002524.6; 2649610.7) |
| GM FI¹ | Day 15 vs Day 1 | 2.95 | (2.669; 3.253) | 1.03 | (0.992; 1.073) |
| GM FI¹ | Day 30 vs Day 1 | 2.94 | (2.676; 3.239) | 1.01 | (0.965; 1.058) |
| % 2-fold² | Day 15 vs Day 1 | 62.8% | (56.57%; 68.82%) | 1.6% | (0.19%; 5.53%) |
| % 2-fold² | Day 30 vs Day 1 | 64.6% | (58.53%; 70.41%) | 1.5% | (0.19%; 5.45%) |
| % 4-fold² | Day 15 vs Day 1 | 37.5% | (31.56%; 43.83%) | 1.6% | (0.19%; 5.53%) |
| % 4-fold² | Day 30 vs Day 1 | 38.4% | (32.50%; 44.58%) | 1.5% | (0.19%; 5.45%) |

Serotype O15

| | | | | | |
|---|---|---|---|---|---|
| GMTs | Day 1 | 1178299.6 | (1056703.9; 1313887.4) | 1188287.9 | (1014882.9; 1391321.1) |
| GMTs | Day 15 | 5790439.7 | (5353107.3; 6263500.7) | 1198106.9 | (1021376.8; 1405416.7) |
| GMTs | Day 30 | 5728512.6 | (5308296.8; 6181993.5) | 1204454.0 | (1026963.7; 1412620.0) |
| GM FI¹ | Day 15 vs Day 1 | 4.97 | (4.461; 5.545) | 1.02 | (0.974; 1.070) |
| GM FI¹ | Day 30 vs Day 1 | 4.98 | (4.477; 5.544) | 1.02 | (0.961; 1.075) |
| % 2-fold² | Day 15 vs Day 1 | 82.6% | (77.37%; 87.07%) | 0.8% | (0.02%; 4.28%) |
| % 2-fold² | Day 30 vs Day 1 | 84.0% | (79.03%; 88.24%) | 1.5% | (0.19%; 5.45%) |
| % 4-fold² | Day 15 vs Day 1 | 59.3% | (52.96%; 65.40%) | 0.8% | (0.02%; 4.28%) |
| % 4-fold² | Day 30 vs Day 1 | 58.6% | (52.34%; 64.57%) | 0.8% | (0.02%; 4.21%) |

Serotype O16

| | | | | | |
|---|---|---|---|---|---|
| GMTs | Day 1 | 1040760.3 | (957123.1; 1131706.2) | 1089930.0 | (964478.4; 1231699.4) |
| GMTs | Day 15 | 5395867.2 | (4945129.5; 5887688.6) | 1068317.6 | (948255.2; 1203581.7) |
| GMTs | Day 30 | 5148889.9 | (4731805.0; 5602738.7) | 1119689.3 | (994125.9; 1261112.1) |
| GM FI¹ | Day 15 vs Day 1 | 5.20 | (4.673; 5.785) | 1.03 | (0.987; 1.083) |
| GM FI¹ | Day 30 vs Day 1 | 5.03 | (4.565; 5.539) | 1.06 | (0.992; 1.124) |
| % 2-fold² | Day 15 vs Day 1 | 86.6% | (81.73%; 90.51%) | 0.8% | (0.02%; 4.28%) |
| % 2-fold² | Day 30 vs Day 1 | 86.3% | (81.56%; 90.23%) | 1.5% | (0.19%; 5.45%) |
| % 4-fold² | Day 15 vs Day 1 | 63.2% | (56.97%; 69.19%) | 0.8% | (0.02%; 4.28%) |
| % 4-fold² | Day 30 vs Day 1 | 62.0% | (55.81%; 67.87%) | 1.5% | (0.19%; 5.45%) |

Serotype O18A

| | | | | | |
|---|---|---|---|---|---|
| GMTs | Day 1 | 1190203.0 | (1087258.8; 1302894.3) | 1302260.8 | (1143295.8; 1483328.6) |
| GMTs | Day 15 | 4279941.4 | (3862457.1; 4742550.6) | 1343837.5 | (1178013.2; 1533004.1) |
| GMTs | Day 30 | 4072329.3 | (3684419.9; 4501079.2) | 1356020.6 | (1188694.1; 1546900.9) |
| GM FI¹ | Day 15 vs Day 1 | 3.65 | (3.282; 4.058) | 1.06 | (1.025; 1.097) |
| GM FI¹ | Day 30 vs Day 1 | 3.41 | (3.095; 3.758) | 1.07 | (1.014; 1.128) |
| % 2-fold² | Day 15 vs Day 1 | 69.2% | (63.08%; 74.80%) | 0.8% | (0.02%; 4.28%) |
| % 2-fold² | Day 30 vs Day 1 | 69.2% | (63.24%; 74.73%) | 1.5% | (0.19%; 5.45%) |
| % 4-fold² | Day 15 vs Day 1 | 47.8% | (41.53%; 54.17%) | 0.0% | (0.00%; 2.84%) |
| % 4-fold² | Day 30 vs Day 1 | 43.0% | (36.90%; 49.19%) | 0.8% | (0.02%; 4.21%) |

Serotype O25B

| | | | | | |
|---|---|---|---|---|---|
| GMTs | Day 1 | 386797.7 | (340192.1; 439788.2) | 374962.9 | (316641.9; 444025.8) |
| GMTs | Day 15 | 2214631.7 | (1915515.0; 2560457.0) | 362651.4 | (304024.4; 432583.7) |
| GMTs | Day 30 | 2124158.2 | (1851850.8; 2436507.4) | 372616.7 | (314118.4; 442009.2) |
| GM FI¹ | Day 15 vs Day 1 | 5.96 | (5.129; 6.937) | 1.02 | (0.965; 1.073) |
| GM FI¹ | Day 30 vs Day 1 | 5.57 | (4.851; 6.389) | 1.04 | (0.963; 1.114) |
| % 2-fold² | Day 15 vs Day 1 | 77.9% | (72.24%; 82.83%) | 0.8% | (0.02%; 4.28%) |
| % 2-fold² | Day 30 vs Day 1 | 77.9% | (72.44%; 82.81%) | 2.3% | (0.48%; 6.60%) |
| % 4-fold² | Day 15 vs Day 1 | 58.9% | (52.56%; 65.02%) | 0.8% | (0.02%; 4.28%) |
| % 4-fold² | Day 30 vs Day 1 | 56.7% | (50.43%; 62.73%) | 1.5% | (0.19%; 5.45%) |

Serotype O75

| | | | | | |
|---|---|---|---|---|---|
| GMTs | Day 1 | 1610037.7 | (1463566.9; 1771167.0) | 1522567.0 | (1323219.9; 1751946.3) |
| GMTs | Day 15 | 3874235.9 | (3529397.7; 4252766.2) | 1527849.3 | (1311522.5; 1779857.8) |
| GMTs | Day 30 | 3753012.6 | (3419195.0; 4119421.0) | 1572632.2 | (1362470.9; 1815211.0) |
| GM FI¹ | Day 15 vs Day 1 | 2.44 | (2.236; 2.671) | 1.02 | (0.977; 1.073) |
| GM FI¹ | Day 30 vs Day 1 | 2.32 | (2.136; 2.526) | 1.04 | (0.979; 1.109) |
| % 2-fold² | Day 15 vs Day 1 | 53.0% | (46.61%; 59.25%) | 1.6% | (0.19%; 5.53%) |
| % 2-fold² | Day 30 vs Day 1 | 51.0% | (44.74%; 57.14%) | 2.3% | (0.48%; 6.60%) |
| % 4-fold² | Day 15 vs Day 1 | 25.3% | (20.06%; 31.12%) | 1.6% | (0.19%; 5.53%) |
| % 4-fold² | Day 30 vs Day 1 | 23.6% | (18.58%; 29.18%) | 2.3% | (0.48%; 6.60%) |

Cohort 2: output for the primary analysis
¹= GM of FI from baseline (Day 1)
²= percentage of participants with fold increase from baseline (Day 1)
CI = Confidence interval;
GM = Geometric mean,
GMT = Geometric mean titer;
FI = Fold increase
95% CI for GMT and GM FI is based on the t-distribution
95% CI for Percentage of Participants With 2- and 4-Fold Increase is obtained using Clopper-Pearson method.

The magnitude of response at day 15 (GMTs) was similar between cohort 1 and 2 for all serotypes.

### Example 8: Novel ExPEC Compositions and Immunogenicity in Rabbits

A new study was designed, which included 2 adapted compositions (also referred to herein as adapted formulations) of ExPEC vaccine (ExPEC9V a and b, Table 17). These adapted formulations are depleted of the serotype O8 polysaccharide and have increased polysaccharide content for the E. coli serotype O75 (ExPEC9V b), since low immunogenicity was observed for these antigens during the analysis of the cohort 1 from the Ph1/2a clinical trial. As a control to verify earlier experiments described above, an ExPEC10V composition was also evaluated. A saline control (unvaccinated group) was also included.

The overall aim of the study was to evaluate whether the changes in the vaccine composition as compared to ExPEC10V described above (depletion of O8 polysaccharide and dose increase specifically for O75 antigen polysaccharide) have any impact on the vaccine-induced immune response. In particular, the aim was to see if increasing the amount of conjugate of E. coli O75 antigen polysaccharide as compared to e.g. conjugate of E. coli O6 antigen polysaccharide in multivalent conjugate vaccine compositions indeed selectively improves the immune response to E. coli of serotype O75, and thus results in a more uniformly elevated immune response across the constituent E. coli serotypes. For that, the antibody responses induced by different formulations of ExPEC vaccine were evaluated in an established pre-clinical model of immunogenicity using New Zealand White rabbits (NZW).

### Experimental Design

New Zealand White rabbits (NZW, 13 weeks of age at start of the study) received 3 intramuscular immunizations (500 μL/injection) with different formulations of ExPEC vaccine or saline administered 2 weeks apart (Table 17). The study contained 4 different groups (Table 18): Group 1 (ExPEC10V) received 8 μg/dose of O1A, O2, O6A, O4, O8, O15, O16, O18 and O75, and 16 μg/dose of O25B (i.e. 0.5 mL of a composition comprising 16 μg/mL of each of O1A, O2, O6A, O4-Glc+, O8, O15, O16, O18A and O75 antigen polysaccharides, and 32 μg/mL of O25B antigen polysaccharide, each antigen polysaccharide separately bioconjugated to EPA carrier protein, see examples above); Group 2 (ExPEC9V a) received 8 μg/dose of O1A, O2, 06A, O4, O15, O16, O18A and O75, and 16 μg/dose of O25B (i.e. 0.5 mL of a composition comprising 16 μg/mL of each of O1A, O2, O6A, O4-Glc+, O15, O16, O18A and O75 antigen polysaccharides, and 32 μg/mL of O25B antigen polysaccharide, each antigen polysaccharide separately bioconjugated to EPA carrier protein, see examples above); Group 3 (ExPEC9V b) received 8 μg/dose of O1A, O2, O6A, O4, O15, O16 and O18A, and 16 μg/dose of O75 and O25B (i.e. 0.5 mL of a composition comprising 16 μg/mL of each of O1A, O2, O6A, O4-Glc+, O15, O16, and O18A antigen polysaccharides, and 32 μg/mL of O25B and O75 antigen polysaccharides, each antigen polysaccharide separately bioconjugated to EPA carrier protein, see examples above).

The buffer used for compounding contained 6.19 mM KH2PO4, 3.81 mM Na2HPO4, 5% sorbitol (w/w), 10 mM Methionine, 0.02% PS80 (w/w), pH 7.0. Animals from the control group (Group 4) received saline (0.9% (w/v) sodium chloride solution). Each experimental group contained 10 animals. Serum antibody levels measured by ELISA (total IgG) were evaluated pre-immunization (day 0) and post-immunization (day 14, 28 and 42).

TABLE 17
Different multivalent ExPEC vaccine compositions.

| Serotypes | ExPEC10V | ExPEC9V a | ExPEC9V b |
|---|---|---|---|
| O1A | 8 | 8 | 8 |
| O2 | 8 | 8 | 8 |
| O4 | 8 | 8 | 8 |
| O6A | 8 | 8 | 8 |
| O8 | 8 | 0 | 0 |
| O15 | 8 | 8 | 8 |
| O16 | 8 | 8 | 8 |
| O18A | 8 | 8 | 8 |
| O25B | 16 | 16 | 16 |
| O75 | 8 | 8 | 16 |
| Total PS | 88 | 80 | 88 |
| (μg) | | | |

TABLE 18
Experimental groups.

| | | | Number |
|---|---|---|---|
| | | Dosing | of |
| Groups | Treatment | at days | animals |
| 1 | ExPEC10V | 0, 14, 28 | 10 |
| 2 | ExPEC9V a (O75:O6 in 1:1 ratio) | 0, 14, 28 | 10 |
| 3 | ExPEC9V b (O75:O6 in 2:1 ratio) | 0, 14, 28 | 10 |
| 4 | Saline | 0, 14, 28 | 10 |

Potential injection site reactions were monitored using Draize scoring system before each dose, approximately 6 hours post-dose on each dosing day, and daily for 7 days after the injection. If case injection site effects were observed, the daily observations were continued until scores returned to 0.

Animals were individually weighed (in grams) once during pre-treatment, daily for 3 days post each dose, once weekly thereafter. Animals were observed at least once daily for ill health, or untoward clinical effects. On dosing days, animals were examined for reaction to treatment before dosing and approximately 6 h after treatment (at same time as injection site examination). Body temperature was monitored using a rectal digital thermometer and recorded pre-dose, 6 h and 24 h after each dose. Where a temperature falls outside the normal range for rabbits of 38-40° C., additional measurements were conducted daily until body temperature returned to be within these values.

Animals received 3 intramuscular immunizations with ExPEC10V, ExPEC9V a, ExPEC9V b, or saline, administered 2 weeks apart. Antibody levels were measured by ELISA at day 0 (pre-vaccination) and days 14, 28 and 42 (post-vaccination).

Serum antibody levels induced by each of the O-antigens included in ExPEC vaccine compositions were analyzed by ELISA using Gen 5 software. OD at 450 nm was analyzed in a 4 parameter (4PL) nonlinear regression model. Half maximal effective concentration (EC50) was calculated for each individual sample based on duplicate 12 step titration curves. Sample results are expressed as EC50 titers.

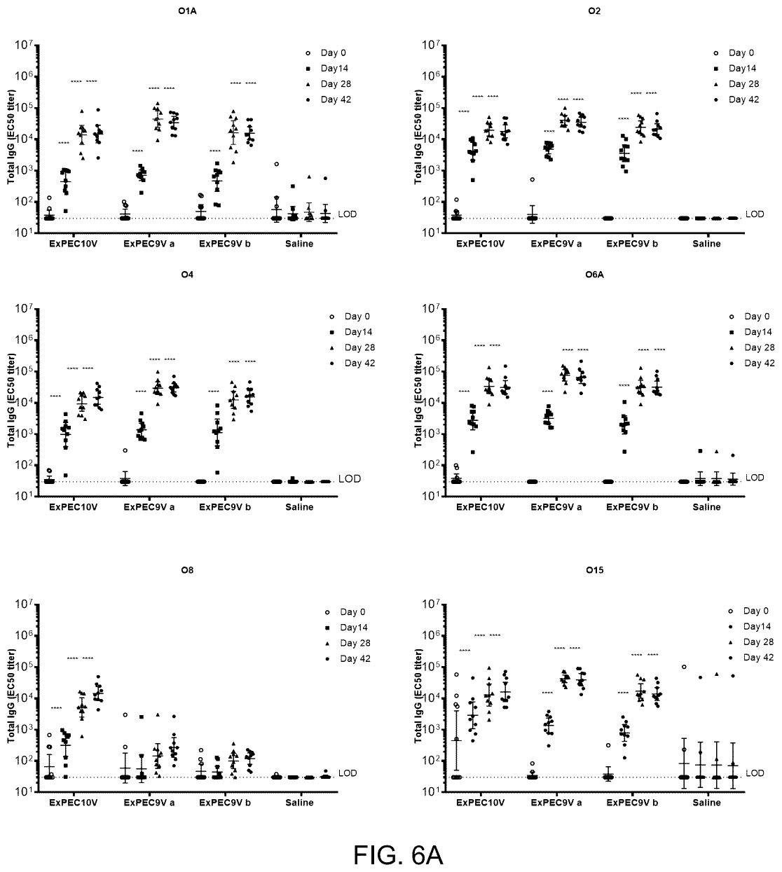

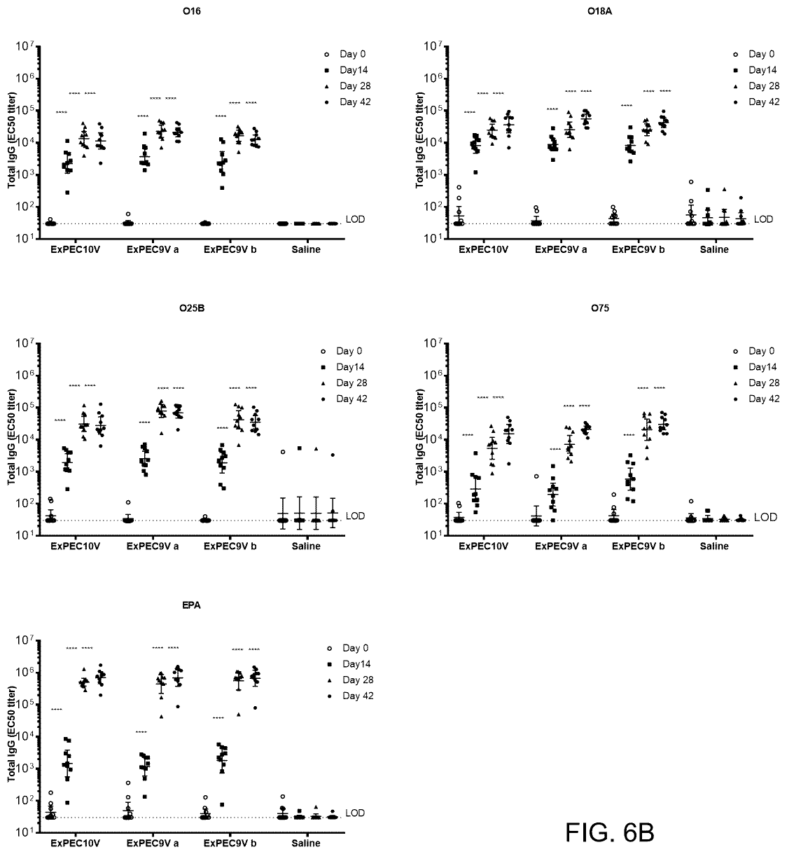

Evaluation of antigen-specific serum antibody responses in NZW rabbits showed that all ExPEC vaccine compositions tested (ExPEC10V, ExPEC9V a and ExPEC9V b) induced a significant increase in antibody responses to all vaccine antigens at day 14, 28 and 42 post-vaccination when compared to the saline control (FIG. 6). The second dose of all ExPEC vaccine compositions tested boosted antibody responses as demonstrated by a significant increase in antigen specific antibody responses from day 14 to day 28 post-vaccination. A further boost effect of the third dose of ExPEC vaccine was evident for certain antigens and compositions.

The geometric mean antibody titers (GMT) of O75 antigen increased with increased concentration of O75 polysaccharide (FIG. 6). ExPEC9V b composition, containing increased concentration of the polysaccharide O75 (16 μg), induced higher O75 GMT and GMT fold-increase at day 14, 28 and 42 post-vaccination when compared to ExPEC9V a and ExPEC10V compositions that contain only 8 μg of O75 polysaccharide (Table 19).

TABLE 19
Evaluation of O75 antibody responses in different ExPEC vaccine compositions.

| | O75 antibody | Time-point post-vaccination | | | |
|---|---|---|---|---|---|
| Treatment | responses | Day 0 | Day 14 | Day 28 | Day 42 |
| ExPEC10V | GMT | 38 | 288 | 5322 | 15052 |
| | 95% CI | 26.7-53.2 | 115.0-720.4 | 2430.2-11653.4 | 7569.2-29930.8 |
| | GMT fold increase | na | 7.6 | 141.2 | 399.4 |
| ExPEC9V a | GMT | 41 | 192 | 7116 | 20798 |
| | 95% CI | 20.1-84.5 | 83.9-439.6 | 3769.1-13436.4 | 16368.0-26425.8 |
| | GMT fold increase | na | 4.7 | 172.7 | 504.7 |
| ExPEC9V b | GMT | 42 | 585 | 20303 | 30269 |
| | 95% CI | 26.7-65.2 | 264.3-1296.6 | 9403.1-43836.5 | 20294.9-45146.3 |
| | GMT fold increase | na | 14.0 | 486.9 | 726.0 |

n.a.: not applicable;
GMT: geometric mean antibody titers;
CI: confidence interval;
fold-increase GMT compared to day 0 (baseline pre-vaccination).

Evaluation of body weight, body temperature and injection site reactions show that all ExPEC vaccine compositions tested were well tolerated by the animals (data not shown).

In conclusion, all ExPEC vaccine compositions tested were immunogenic in NZW rabbits, significant increase in antigen-specific serum antibody responses were observed at day 14, 28 and 42 post-vaccination when compared to saline control. All ExPEC vaccine compositions tested were able to boost polysaccharide specific-antibody responses. In addition, all ExPEC vaccine compositions tested were well tolerated by the animals, no vaccine-related adverse events were observed. Importantly, ExPEC9V b composition, containing increased concentration of O75 polysaccharide, induced higher O75 antibody responses (GMT and GMT fold-increase) when compared to compositions that contain lower concentration of O75 polysaccharide (ExPEC9Va and ExPEC10V). The increased dose of O75 in the ExPEC9V b composition thus improves the immune response to the O75 serotype, and therefore such a composition leads to a more uniformly elevated immune response across the serotypes in the ExPEC9V b composition.

### Example 9. Phase 3 Efficacy Trial in Humans with Novel ExPEC9V Composition

An efficacy study in humans is conducted with the 9-valent ExPEC vaccine composition that has an increased concentration of O75 antigen polysaccharide. The title of the study is: “Randomized, Double-blind, Placebo-controlled, Multicenter Phase 3 Study to Assess the Efficacy, Safety And Immunogenicity of Vaccination With ExPEC9V in the Prevention of Invasive Extraintestinal Pathogenic Escherichia coli Disease in Adults Aged 60 Years And Older with a History of Urinary Tract Infection in the Past 2 Years.”, and it will be referred to as the ‘BAC3001’ study herein.

ExPEC9V is a 9-valent vaccine candidate in development for the prevention of invasive extraintestinal pathogenic Escherichia coli (ExPEC) disease (IED) in adults 60 years of age and older. ExPEC9V consists of the O-antigen polysaccharides (PSs) of the ExPEC serotypes O1A, O2, O4, O6A, O15, O16, O18A, O25B, and O75 separately bioconjugated to the carrier protein, a genetically detoxified form of exotoxin A (EPA) derived from Pseudomonas aeruginosa, as described in detail in the previous examples. ExPEC9V vaccine covers the 9 indicated E. coli serotypes, which are among the most prevalent O-serotypes of ExPEC that are responsible for about 6500 of all IED.

### Objectives and Endpoints

| | |
|---|---|
| Objectives | Endpoints |

Primary

| | |
|---|---|
| To demonstrate the efficacy of ExPEC9V compared | First IED event, with microbiological |
| to placebo in the prevention of the first IED event, | confirmation from blood, other sterile sites, or |
| with microbiological confirmation from blood, other | urine, caused by ExPEC9V O-serotypes O1A, |
| sterile sites, or urine, caused by ExPEC9V | O2, O4, O6A, O15, O16, O18A, O25B, and |
| O-serotypes O1A, O2, O4, O6A, O15, O16, O18A, | O75 |
| O25B, and O75 | |
| To demonstrate the efficacy of ExPEC9V compared | First IED event, with microbiological |
| to placebo in the prevention of the first IED event, | confirmation from blood or other sterile sites, |
| with microbiological confirmation from blood or | caused by ExPEC9V O-serotypes |
| other sterile sites, caused by ExPEC9V O-serotypes | |

Secondary

| | |
|---|---|
| To demonstrate the efficacy of ExPEC9V compared | All IEDs (including multiple IEDs per |
| to placebo in the prevention of all IEDs (including | participant) caused by ExPEC9V O-serotypes |
| multiple IEDs per participant) caused by ExPEC9V | |
| O-serotypes | |
| To demonstrate the efficacy of ExPEC9V compared | First hospitalized IED event caused by |
| to placebo in the prevention of the first hospitalized | ExPEC9V O-serotypes |
| IED event caused by ExPEC9V O-serotypes | |
| To demonstrate the efficacy of ExPEC9V compared | First IED event meeting criteria for sepsis |
| to placebo in the prevention of the first IED event | caused by ExPEC9V O-serotypes |
| meeting criteria for sepsis caused by ExPEC9V | |
| O-serotypes | |
| To demonstrate the efficacy of ExPEC9V compared | First bacteremic IED event caused by |
| to placebo in the prevention of the first bacteremic | ExPEC9V O-serotypes |
| (ie, microbiological confirmation from blood) IED | |
| event caused by ExPEC9V O-serotypes | |
| To demonstrate the efficacy of ExPEC9V compared | First pyelonephritis event caused by ExPEC9V |
| to placebo in the prevention of the first | O-serotypes |
| pyelonephritis event caused by ExPEC9V | |
| O-serotypes | |
| To demonstrate the efficacy of ExPEC9V compared | First UTI event caused by ExPEC9V |
| to placebo in the prevention of the first UTI event | O-serotypes |
| caused by ExPEC9V O-serotypes | |
| To demonstrate the efficacy of ExPEC9V compared | All UTIs (including multiple UTIs per |
| to placebo in the prevention of all UTIs (including | participant) caused by ExPEC9V O-serotypes |
| multiple UTIs per participant) caused by ExPEC9V | |
| O-serotypes | |
| To demonstrate the efficacy of ExPEC9V compared | First IED event caused by any ExPEC |
| to placebo in the prevention of the first IED event | O-serotype |
| caused by any ExPEC O-serotype | |
| To demonstrate the efficacy of ExPEC9V compared | First pyelonephritis event caused by any |
| to placebo in the prevention of the first | ExPEC O-serotype |
| pyelonephritis event caused by any ExPEC | |
| O-serotype | |
| To demonstrate the efficacy of ExPEC9V compared | First UTI event caused by any ExPEC |
| to placebo in the prevention of the first UTI event | O-serotype |
| caused by any ExPEC O-serotype | |
| To evaluate the immunogenicity of ExPEC9V in the | Antibody titers to vaccine O-serotype antigens |
| Immunogenicity Subset | in the Immunogenicity Subset, as determined |
| | by multiplex electrochemiluminescent (ECL)- |
| | based immunoassay and multiplex |
| | opsonophagocytic killing assay (MOPA) on |
| | Day 30, Day 181, Year 1, Year 2, and Year 3 |
| To evaluate the safety and reactogenicity of | Solicited local and systemic AEs (collected |
| ExPEC9V | until 14 days post-vaccination [from Day 1 to |
| | Day 15] in the Safety Subset) |
| | Unsolicited AEs (collected until 29 days post- |
| | vaccination [from Day 1 to Day 30] in all |
| | participants) |
| | Serious adverse events (SAEs) in all |
| | participants |
| To evaluate the preservation of health status and | SF-36 and EQ-5D-5L responses at scheduled |
| health-related quality of life (HRQoL) of ExPEC9V | timepoints |
| compared to placebo as measured by the Short Form- | |
| 36 (SF-36) survey and the 5-level EuroQol | |
| 5-Dimension questionnaire (EQ-5D-5L) Descriptive | |
| System | |
| To evaluate the impact of IED, caused by ExPEC9V | |
| O-serotypes, on physical and mental health, and | |
| overall HRQoL as measured by the SF-36 | |
| To evaluate the impact of UTI, caused by ExPEC9V | |
| O-serotypes, on physical and mental health, and | |
| overall HRQoL, as measured by the SF-36 | |
| To assess the degree of frailty in participants who | Frailty index as a measure of frailty at baseline, |
| received ExPEC9V versus placebo at baseline, Year | Year 1, Year 2, Year 3, and at the time of an |
| 1, Year 2, Year 3, and at the time of an IED | IED |
| To determine the medical resource utilization for | Medical resource utilization for IED events |
| IED and UTI events caused by any ExPEC | Medical resource utilization for UTI events |
| O-serotype | (Immunogenicity Subset only) and acute |
| | bacterial prostatitis (ABP) events |
| | (Immunogenicity Subset only) |
| | Hospitalization and length of stay in hospital for |
| | IED, UTI, or ABP events |
| To determine the mortality associated with IED | IED-related and all-cause mortality |

Exploratory

| | |
|---|---|
| To evaluate the efficacy of ExPEC9V compared to | First IED event caused by ExPEC9V |
| placebo in the prevention of the first IED event | O-serotypes showing resistance to ≥1, ≥2, and |
| caused by ExPEC9V O-serotypes resistant to ≥1, ≥2, | ≥3 antibiotics in the microbiological profile |
| and ≥3 antibiotics | |
| To evaluate the efficacy of ExPEC9V compared to | First uncomplicated UTI event caused by |
| placebo in the prevention of first uncomplicated, | ExPEC9V O-serotypes |
| complicated, and recurrent UTI events caused by | First complicated UTI event caused by |
| ExPEC9V O-serotypes | ExPEC9V O-serotypes |
| | First recurrent UTI (rUTI) event caused by |
| | ExPEC9V O-serotypes |
| | First recurrent uncomplicated UTI event caused |
| | by ExPEC9V O-serotypes |
| To evaluate the efficacy of ExPEC9V compared to | First ABP event caused by ExPEC9V |
| placebo in the prevention of the first ABP event | O-serotypes |
| caused by ExPEC9V O-serotypes | |
| To evaluate antibody titers to vaccine O-serotype | Antibody titers for ExPEC9V, determined by |
| antigens in participants with IED | multiplex ECL-based immunoassay and MOPA |
| | assays in participants with IED, with sera |
| | obtained at the time of diagnosis of the |
| | suspected IED |
| To evaluate the severity of IED | Severity of IED (presence of sepsis or septic |
| | shock) in participants with IED |
| To identify a correlate of protection for the vaccine- | Antibody titers for ExPEC9V determined by |
| mediated immune response associated with vaccine | multiplex ECL-based immunoassay and MOPA |
| efficacy | assays at Day 1 pre-vaccination, Day 30 and at |
| | the time of diagnosis of the suspected IED (all |
| | participants) or at the time of a UTI or ABP |
| | event (Immunogenicity Subset only), in |
| | association with vaccine efficacy |
| To evaluate the efficacy of ExPEC9V compared to | First invasive disease event caused by |
| placebo in the prevention of the first invasive disease | P. aeruginosa |
| event caused by P. aeruginosa and first | First P. aeruginosa UTI event |
| P. aeruginosa UTI | |

### Overall Design

This is a randomized, double-blind, placebo-controlled, parallel-group, multicenter, interventional Phase 3 study to be conducted in approximately 18,556 medically stable adults aged ≥60 years and with a history of UTI in the past 2 years.

All participants are enrolled and randomized in parallel in a 1:1 ratio to either ExPEC9V or placebo and receive the study vaccine (0.5 mL intramuscular injection (in deltoid)) on Day 1. The final analysis for the first primary endpoint (with microbiological confirmation from blood, other sterile sites, or urine) occurs when 72 vaccine serotype IED events have been observed in the study or at the latest when the last participant has been followed up for 36 months post-vaccination. The second primary endpoint (with microbiological confirmation from blood or other sterile sites) is tested in a hierarchical manner when the first primary endpoint has shown statistical significance. For the final analysis of the second primary endpoint (with microbiological confirmation from blood or other sterile sites), 53 vaccine serotype IED events according to the second primary endpoint have to be observed. The ExPEC9V vaccine composition comprises 16 μg/mL of each of O1A, O2, O6A, O4-Glc+, O15, O16, and O18A antigen polysaccharides and 32 μg/mL of O25B and O75 antigen polysaccharides, each antigen polysaccharide separately bioconjugated to EPA carrier protein, see examples above (e.g. this is the same composition used in group 3 of the rabbit immunizations of example 8 above; the excipients are the same as described for the ExPEC10V vaccine used in the BAC1001 study of example 7). Placebo is 0.9% w/v sodium chloride.

### Safety Evaluations

Key safety assessments include SAEs, physical examination, and vital signs. In addition, solicited local and systemic AEs and unsolicited AEs are recorded for participants in the “Safety Subset”, ie, approximately 4,000 participants from selected study sites who have given informed consent prior to randomization for the additional assessments.

### Immunogenicity Evaluations

For assessment of immunogenicity, IgG antibody levels elicited by the vaccine against each of the 9 vaccine O-serotypes and the carrier protein EPA are measured by a multiplex ECL-based immunoassay and serotype-specific functional antibodies are measured by a MOPA. Immunogenicity analysis is performed for participants in the “Immunogenicity Subset”, ie, for approximately 1,200 participants from selected study sites who have given informed consent prior to randomization for additional immunogenicity assessments, and for all participants with a confirmed IED event, and for participants in the Immunogenicity Subset with a UTI or acute bacterial prostatitis (ABP) event.

### Efficacy Evaluations

The primary objective is to demonstrate efficacy of ExPEC9V compared to placebo in the prevention of the first IED events caused by ExPEC serotypes O1A, O2, O4, O6A, O15, O16, O18A, O25B, and O75. Events defined as IED, UTI, or ABP are collected for all participants for the entire study duration (1,096 days; 3 years). During study follow-up, participants are expected to inform the study site, as soon as possible, if they experience any signs or symptoms of UTI or ABP, or if they experience any new onset or worsening of symptoms that could be caused by a systemic infection. In addition, all participants will be questioned about past hospitalizations or medical events that could have been UTIs, ABPs, or potential IEDs, and that were not captured by the site in real time. Medical resource utilization data associated with medical encounters related to IED (all participants) and for UTI or ABP (Immunogenicity Subset only) are collected. Two patient-reported outcome (PRO) instruments are used to measure Health-Related Quality of Life: the SF-36 version 2 and the EQ-5D-5L. In addition, frailty is assessed using the SPPB and the Frailty Index Score.

### Immunogenicity Evaluations

For assessment of immunogenicity, IgG antibody levels elicited by the vaccine against each of the 9 vaccine O-serotypes and the carrier protein EPA is measured by a multiplex ECL-based immunoassay and serotype-specific functional antibodies is measured by a MOPA. Immunogenicity analysis is performed for participants in the “Immunogenicity Subset”, ie, for approximately 1,200 participants from selected study sites who have given informed consent prior to randomization for additional immunogenicity assessments, and for all participants with a confirmed IED and for participants in the Immunogenicity Subset with an UTI or an ABP event. Day 1 and Day 30 immunogenicity blood samples are collected from all participants.

### Safety Evaluations

Key safety assessments include SAEs, physical examination, and vital signs. In addition, solicited local and systemic AEs and unsolicited AEs are recorded for participants in the “Safety Subset”, ie, approximately 4,000 participants from selected study sites who have given informed consent prior to randomization for the additional assessments.

### Efficacy Analyses

The primary analysis of the first primary endpoint evaluates the number of participants with at least 1 IED event caused by ExPEC serotypes O1A, O2, O4, O6A, O15, O16, O18A, O25B, and O75 with onset at least 29 days after vaccination in the active vaccine (ExPEC9V) group compared to the placebo group in the per protocol efficacy (PPE) population. The primary analysis of the second primary endpoint evaluates the number of participants with at least 1 IED event with microbiological confirmation in blood or other sterile sites caused by ExPEC serotypes O1A, O2, O4, O6A, O15, O16, O18A, O25B, and O75 with onset at least 29 days after vaccination (from Day 30) in the active vaccine (ExPEC9V) group compared to the placebo group in the PPE population. The follow-up time is also taken into account. For participants with an IED event, the follow-up time is defined as the time between vaccination and the occurrence of the first event. For participants without an IED event, it is the time between vaccination and last visit (or last contact for participants that discontinued the study). The null hypothesis of VE≤20% is tested versus the alternative hypothesis VE>20%.

A stagewise hierarchical testing strategy is applied to the following endpoints:

### Primary:

First IED, with microbiological confirmation from blood, other sterile sites, or urine, caused by ExPEC9V O-serotypes
First IED with microbiological confirmation from blood or other sterile sites caused by ExPEC9V O-serotypes

### Secondary:

All IEDs (including multiple IEDs per participant) caused by ExPEC9V O-serotypes
First hospitalized IED event caused by ExPEC9V O-serotypes
First IED event meeting criteria for sepsis caused by ExPEC O-serotypes
First bacteremic IED event caused by ExPEC9V O-serotypes
First pyelonephritis event caused by ExPEC9V O-serotypes
First UTI caused by ExPEC9V O-serotypes
All UTIs (including multiple UTIs per participant) caused by ExPEC9V O-serotypes
First IED event caused by any ExPEC O-serotype
First pyelonephritis event caused by any ExPEC O-serotype
First UTI caused by any ExPEC O-serotype

In this testing strategy, the second primary endpoint is only tested when the first primary endpoint showed statistical significance. When the first primary endpoint has shown statistical significance with 72 events, an interim analysis is performed for the second primary endpoint (with microbiological confirmation from blood or other sterile sites) if less than 53 events are observed at that time. If the second primary endpoint is not significant, the final analysis of the second primary endpoint is performed when 53 vaccine serotype IED events according to the second primary endpoint definition have been observed. Further, the first key secondary endpoint is only tested when both primary endpoints showed statistical significance. The first key secondary endpoint includes all ExPEC9V IED events with onset of at least 29 days after vaccination (PPE) until the end of the follow-up period.

A participant is considered to have IED if the below criteria are met and confirmed by the independent endpoint adjudication committee (IEAC).

An adult participant diagnosed with IED is any participant with:
signs and symptoms of systemic bacterial infection as indicated by the presence of any of the following:
   temperature<36.0° C. (96.8° F.) or >38.0° C. (100.4° F.),
   tachycardia (HR)>90 bpm,
   tachypnoea (RR)>20 breaths/minute, or
   WBC count<4 or >12×10⁹/L or 10% immature (band) forms,
   AND
microbiological confirmation by culture of:
   E. coli in blood or in any other sterile site (e.g. cerebrospinal fluid [CSF], pleura), AND/OR
   E. coli in urine (colony forming units/mL≥10⁵) with UTI sign/symptoms and with no other identifiable site of infection and with an acute change in total sequential organ failure assessment (SOFA) score≥2 points from baseline.

For the primary and secondary endpoints, with IED caused by any of the ExPEC9V O-serotypes (ie, O1A, O2, O4, O6A, O15, O16, O18A, O25B, or O75), the O-serotyping has to be available and the IED has to be confirmed as being O1A, O2, O4, O6A, O15, O16, O18A, O25B, or O75 positive. For the secondary endpoints related to any ExPEC O-serotype, no O-serotyping has to be available. Cases with multiple pathogens in the culture results are not considered for the primary or secondary endpoints if the systemic infection cannot be attributed only to E. coli, as per IEAC judgment. Cases with mixed pathogens and the presence of E. coli are included in sensitivity analyses.

If a participant has contacted their physician and/or study site with signs and symptoms of UTI, the participant is defined as having a symptomatic UTI if the below criteria are met and confirmed by the IEAC for cases of pyelonephritis.

An adult with documented pyuria (white blood cell (WBC) count≥10 cells/mm³) and microbiological confirmation by culture of E. coli in urine (colony forming units≥10⁵/mL) with one of the following definitions for signs and symptoms:
for uncomplicated UTI, at least 2 of the following signs or symptoms:
   dysuria,
   urinary frequency,
   urinary urgency, or
   suprapubic pain.
for complicated UTI (cUTI):
   at least 2 of the following signs or symptoms:
      chills, rigors or warmth associated with fever (eg, temperature≥38.0° C.),
      dysuria, urinary frequency or urinary urgency,
      lower abdominal pain or pelvic pain, or
      nausea or vomiting,
   AND,
   at least 1 of the following complicating factors:
      history of urinary retention (in male participants),
      current indwelling urinary catheter,
      obstructive uropathy, or
      any relevant functional or anatomical abnormality.
for pyelonephritis (regardless of underlying abnormality of the urinary tract) with at least 2 of the following symptoms:
   chills, rigors, or warmth associated with fever (eg, temperature≥38.0° C.), or
   flank pain or tenderness in the costovertebral angle on physical examination, or
   nausea or vomiting.
for recurrent UTI (rUTI):
   recurrence of uncomplicated and/or complicated UTIs with a frequency of two UTIs in the last six months, or
   recurrence of uncomplicated and/or complicated UTIs with a frequency of at least three UTIs per year.

For the secondary endpoints, with UTI caused by any of the ExPEC9V O-serotypes (ie, O1A, O2, O4, O6A, O15, O16, O18A, O25B, or O75), the O-serotyping has to be available and the UTI has to be confirmed as being O1A, O2, O4, O6A, O15, O16, O18A, O25B, or O75 positive. For the secondary endpoints, related to any ExPEC O-serotype, no O-serotyping has to be available. Cases with multiple pathogens in the culture results and with the presence of E. coli are not considered for the secondary endpoints. These cases are included in sensitivity analyses.

As an exploratory endpoint, participants meeting the above definition of IED (except the microbiological confirmation of E. coli) or UTI but with (urine in case of UTI) samples positive for P. aeruginosa (colony forming units≥10⁵/mL in case of UTI) are reported to have invasive disease due to P. aeruginosa, or P. aeruginosa UTI, respectively. No serotyping is performed for P. aeruginosa. Samples with mixed pathogens with the presence of P. aeruginosa are not considered for this exploratory endpoint.

As an exploratory efficacy endpoint for ABP, events caused by ExPEC serotypes O1A, O2, O4, O6A, O15, O16, O18A, O25B, and O75 are reported.

If a participant contacts their physician and/or study site with signs and symptoms of ABP, the participant is defined as having a symptomatic ABP if the below criteria are met.

An adult with documented pyuria (WBC count≥10 cells/mm³) and microbiological confirmation by culture of E. coli in urine (colony forming units≥10⁵/mL) with fever and an acute onset of the following:
irritative voiding symptoms:
   dysuria,
   urinary frequency,
   urinary urgency, or
obstructive voiding symptoms:
   hesitancy,
   incomplete voiding,
   straining to urinate,
   weak stream, and
suprapubic, rectal, or perineal pain.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Sequences

(Glycosylation consensus sequence)
SEQ ID NO: 1
Asn-X-Ser(Thr),
wherein X can be any amino acid except Pro
(Optimized glycosylation consensus sequence)
SEQ ID NO: 2
Asp(Glu)-X-Asn-Z-Ser(Thr),
wherein X and Z are independently selected from any amino acid except Pro
(EPA carrier protein comprising 4 glycosylation consensus sequences (EPA-4)
SEQ ID NO: 3
G SGGGDQNATG SGGGKLAEEA FDLWNECAKA CVLDLKDGVR SSRMSVDPAI ADTNGQGVLH
YSMVLEGGND ALKLAIDNAL SITSDGLTIR LEGGVEPNKP VRYSYTRQAR GSWSLNWLVP IGHEKPSNIK
VFIHELNAGN QLSHMSPIYT IEMGDELLAK LARDATFFVR AHESNEMQPT LAISHAGVSV VMAQAQPRRE
KRWSEWASGK VLCLLDPLDG VYNYLAQQRC NLDDTWEGKI YRVLAGNPAK HDLDIKDNNN STPTVISHRL
HFPEGGSLAA LTAHQACHLP LEAFTRHRQP RGWEQLEQCG YPVQRLVALY LAARLSWNQV DQVIRNALAS
PGSGGDLGEA IREQPEQARL ALTLAAAESE RFVRQGTGND EAGAASADVV SLTCPVAKDQ NRTKGECAGP
ADSGDALLER NYPTGAEFLG DGGDVSFSTR GTQNWTVERL LQAHRQLEER GYVFVGYHGT FLEAAQSIVF
GGVRARSQDL DAIWRGFYIA GDPALAYGYA QDQEPDARGR IRNGALLRVY VPRWSLPGFY RTGLTLAAPE
AAGEVERLIG HPLPLRLDAI TGPEEEGGRV TILGWPLAER TVVIPSAIPT DPRNVGGDLD PSSIPDKEQA
ISALPDYASQ PGKPPREDLK LGSGGGDQNA T
(O4 GtrS amino acid sequence)
SEQ ID NO: 4
MNNLIMNNWCKLSIFIIAFILLWLRRPDILTNAQFWAEDSVFWYKDAYENGFLSSLTTPRNGYFQTVSTFIVGLTAL
LNPDYAPFVSNFFGIMIRSVIIWFLFTERFNFLTLTTRIFLSIYFLCMPGLDEVHANITNAHWYLSLYVSMILIARN
PSSKSWRFHDIFFILLSGLSGPFIIFILAASCFKFINNCKDHISVRSFINFYLRQPYALMIVCALIQGTSIILTFNG
TRSSAPLGFSFDVISSIISSNIFLFTFVPWDIAKAGWDNLLLSYFLSVSILSCAAFVFVKGTWRMKVFATLPLLIII
FSMAKPQLTDSAPQLPTLINGQGSRYFVNIHIAIFSLLCVYLLECVRGKVATLFSKTYLTILLFVMGCLNFVITPLP
NMNWREGATLINNAKTGDVISIQVLPPGLTLELRKK
(Example O4 gtrS nucleic acid sequence)
SEQ ID NO: 5
ATGAATAATTTAATTATGAATAACTGGTGTAAATTATCTATATTTATTATTGCATTTATTTTGCTATGGCTTAGAAG
GCCGGATATACTCACAAACGCACAATTTTGGGCAGAAGATTCCGTTTTCTGGTATAAGGACGCCTATGAGAACGGAT
TCTTAAGTTCACTAACAACGCCTAGGAATGGGTATTTCCAGACTGTTTCTACATTTATAGTTGGTCTGACTGCTTTA
TTAAATCCAGATTATGCACCTTTTGTTTCTAATTTTTTTGGCATAATGATTCGCTCAGTAATTATATGGTTTTTATT
TACAGAAAGATTCAACTTCCTCACATTGACTACTAGGATTTTCTTATCTATTTATTTTCTATGCATGCCTGGATTGG
ATGAAGTTCATGCAAATATAACAAATGCACATTGGTATTTGTCATTATATGTATCAATGATCCTGATAGCTCGCAAT
CCAAGTTCAAAATCATGGAGGTTTCATGATATATTCTTTATCTTGCTATCCGGGCTCAGTGGCCCATTTATAATTTT
CATTTTAGCAGCTTCATGCTTTAAATTTATAAATAATTGTAAAGATCATATTAGTGTAAGATCTTTCATAAATTTCT
ACTTGCGTCAGCCATACGCATTAATGATTGTTTGCGCTTTAATTCAAGGAACTTCTATAATTCTAACTTTCAATGGC
ACACGTTCCTCAGCACCGCTAGGATTCAGTTTTGATGTGATTTCGTCTATTATATCATCGAATATTTTTTTATTTAC
ATTTGTCCCATGGGATATTGCAAAGGCTGGGTGGGATAATTTACTGTTATCTTATTTTTTGTCTGTTTCGATTTTGT
CGTGTGCGGCCTTTGTTTTTGTTAAAGGTACGTGGCGAATGAAAGTATTTGCAACTTTACCATTGCTAATTATAATA
TTTTCAATGGCAAAACCACAATTGACAGACTCGGCACCTCAATTGCCAACACTTATTAATGGGCAAGGTTCAAGATA
CTTCGTAAATATACATATTGCGATATTCTCTTTGCTATGTGTTTACTTACTTGAGTGCGTCAGGGGGAAAGTGGCAA
CTTTATTTTCCAAAATATACTTAACAATTTTGCTATTCGTGATGGGATGTTTGAATTTTGTTATCACCCCACTCCCA
AACATGAACTGGAGGGAAGGTGCTACTTTGATTAATAATGCAAAAACTGGTGATGTCATTTCGATTCAAGTGCTACC
ACCTGGCCTAACACTTGAACTAAGGAAAAAATAA
(Example PglB sequence (‘wild-type’)
SEQ ID NO: 6
MLKKEYLKNPYLVLFAMITLAYVFSVFCRFYWVWWASEFNEYFFNNQLMIISNDGYAFAEGARDMIAGFHQPNDLSY
YGSSLSALTYWLYKITPFSFESIILYMSTFLSSLVVIPTILLANEYKRPLMGFVAALLASIANSYYNRTMSGYYDTD
MLVIVLPMFILFFMVRMILKKDFFSLIALPLFIGIYLWWYPSSYTLNVALIGLFLIYTLIFHRKEKIFYIAVILSSL
TLSNIAWFYQSAIIVILFALFALEQKRLNFMIIGILGSATLIFLILSGGVDPILYQLKFYIFRSDESANLTQGFMYF
NVNQTIQEVENVDLSEFMRRISGSEIVFLFSLFGFVWLLRKHKSMIMALPILVLGFLALKGGLRFTIYSVPVMALGF
GFLLSEFKAIMVKKYSQLTSNVCIVFATILTLAPVFIHIYNYKAPTVFSQNEASLLNQLKNIANREDYVVTWWDYGY
PVRYYSDVKTLVDGGKHLGKDNFFPSFALSKDEQAAANMARLSVEYTEKSFYAPQNDILKTDILQAMMKDYNQSNVD
LFLASLSKPDFKIDTPKTRDIYLYMPARMSLIFSTVASFSFINLDTGVLDKPFTFSTAYPLDVKNGEIYLSNGVVLS
DDFRSFKIGDNVVSVNSIVEINSIKQGEYKITPIDDKAQFYIFYLKDSAIPYAQFILMDKTMFNSAYVQMFFLGNYD
KNLFDLVINSRDAKVFKLKI
(example gtrA amino acid sequence; E. coli W3110 yfdG, GenBank: BAA16209.1)
SEQ ID NO: 7
MLKLFAKYTSIGVLNTLIHWVVFGVCIYVAHTNQALANFAGFVVAVSFSFFANAKFTFKASTTTMRYMLYVGFMGTL
SATVGWAADRCALPPMITLVTFSAISLVCGFVYSKFIVFRDAK
(example gtrB amino acid sequence-E. coli W3110 yfdH, GenBank: BAA16210.1)
SEQ ID NO: 8
MKISLVVPVFNEEEAIPIFYKTVREFEELKSYEVEIVFINDGSKDATESIINALAVSDPLVVPLSFTRNFGKEPALF
AGLDHATGDAIIPIDVDLQDPIEVIPHLIEKWQAGADMVLAKRSDRSTDGRLKRKTAEWFYKLHNKISNPKIEENVG
DFRLMSRDVVENIKLMPERNLFMKGILSWVGGKTDIVEYVRAERIAGDTKFNGWKLWNLALEGITSFSTFPLRIWTY
IGLVVASVAFIYGAWMILDTIIFGNAVRGYPSLLVSILFLGGIQMIGIGVLGEYIGRTYIETKKRPKYIIKRVKK
(example O4 rfb locus nucleotide sequence-O4-EPA production strain BVEC-L-
00684f)
SEQ ID NO: 9
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGTGAAAATACTTGTTACTGGTGGCGCAGGATTTATTGGTTCAG
CTGTAGTTCGTCACATTATAAATAATACGCAGGATAGTGTTGTTAATGTCGATAAATTAACGTACGCCGGAAACCGG
GAATCACTTGCTGATGTTTCTGATTCTGAACGCTATGTTTTTGAACATGCGGATATTTGCGATGCACCTGCAATGGC
ACGGATTTTTGCTCAGCATCAGCCGGATGCAGTGATGCACCTGGCTGCTGAAAGCCATGTTGACCGTTCAATTACAG
GCCCTGCGGCATTTATTGAAACCAATATTGTTGGTACTTATGTCCTTTTGGAAGCCGCTCGCAATTACTGGTCTGCT
CTTGATAGCGACAAGAAAAATAGCTTCCGTTTTCATCATATTTCTACTGACGAAGTATATGGTGATTTGCCTCATCC
TGACGAGGTAAATAATACAGAAGAATTACCCTTATTTACTGAGACAACAGCTTACGCGCCAAGCAGCCCTTATTCCG
CATCCAAAGCATCCAGCGATCATTTAGTCCGCGCGTGGAAACGTACCTATGGTTTACCGACCATTGTGACTAATTGC
TCTAACAATTATGGTCCTTATCATTTCCCGGAAAAATTGATTCCATTGGTTATTCTCAATGCTCTGGAAGGTAAAGC
ATTACCTATTTATGGTAAAGGGGATCAAATTCGCGACTGGCTGTATGTTGAAGATCATGCGCGTGCGTTATATACCG
TCGTAACCGAAGGTAAAGCGGGTGAAACTTATAACATTGGTGGGCACAACGAAAAGAAAAACATAGATGTAGTGCTC
ACTATTTGTGATTTGCTGGATGAGATTGTACCGAAAGAGAAATCTTATCGTGAGCAAATCACTTATGTTGCCGATCG
TCCGGGACACGATCGCCGTTATGCGATTGATGCTGAGAATATTGGTCGCGAATTGGGATGGAAACCACAGGAAACGT
TTGAGAGCGGGATTCGGAAGACAGTGGAATGGTATCTGTCCAATACAAAATGGGTTGATAATGTGAAAAGTGGTGCC
TATCAATCGTGGATTGAAGAGAACTATGAGGGCCGCCAGTAATGAATATCCTCCTTTTTGGCAAAACAGGGCAGGTA
GGTTGGGAACTACAGCGTGCTCTGGCACCTCTGGGTAACTTGATTGCTCTTGATGTTCATTCCACTGATTATTGTGG
CGATTTCAGTAACCCCGAAGGTGTGGCTGAAACCGTCAAAAAAATTCGCCCAGATGTTATTGTTAATGCTGCTGCTC
ATACCGCGGTAGATAAGGCTGAGTCAGAACCAGAATTTGCACAATTACTCAATGCGACCAGCGTTGAAGCAATTGCA
AAAGCGGCTAATGAAGTTGGGGCTTGGGTAATTCATTACTCAACTGACTACGTCTTCCCTGGAAATGGCGACATGCC
ATGGCTCGAGACTGATGTAACCGCTCCGCTCAATGTTTATGGCAAAACCAAATTGGCTGGAGAAAGAGCATTACAAG
AACATTGCGCAAAGCATCTTATTTTCCGTACCAGCTGGGTATATGCAGGTAAAGGAAATAACTTTGCCAAAACAATG
TTACGTCTGGCAAAAGAGCGCGAAGAACTGGCTGTGATAAACGATCAGTTTGGCGCACCAACAGGTGCTGAATTGCT
GGCTGATTGCACCGCTCATGCCATTCGCGTGGCATTAAAAAAACCAGAAGTTGCTGGCTTGTACCATCTGGTAGCAA
ATGGCACAACAACCTGGCACGATTACGCCGCGCTAGTATTCGAAGAAGCCCGTAAAGCAGGGATTGACCTTGCACTT
AACAAACTCAACGCCGTACCAACAACGGCTTATCCTACTCCAGCCCGCCGTCCTCATAATTCTCGCCTCAATACCGA
AAAGTTTCAGCAGAACTTTGCGCTTGTCTTGCCTGACTGGCAGGTGGGCGTGAAACGTATGCTCAACGAATTATTTA
CGACTACGGCAATTTAACAAATTTTTGCATCTCGCTCATGATGCCAGAGCGGGATGAATTAAAAGGAATGGTGAAAT
GAAAACGCGTAAAGGTATTATTCTGGCTGGTGGTTCCGGCACTCGTCTTTATCCTGTGACGATGGCAGTGAGTAAAC
AACTGCTGCCGATTTATGATAAGCCGATGATTTATTATCCGCTTTCAACGCTTATGTTAGCGGGTATTCGCGATATT
CTTATTATCAGTACGCCACAGGATACACCGCGTTTCCAACAATTGTTGGGGGACGGGAGTCAGTGGGGGCTTAATCT
ACAGTATAAAGTACAACCGAGTCCGGATGGCCTGGCGCAAGCGTTTATTATTGGTGAAGACTTTATTGGTGGTGATG
ATTGTGCACTCGTACTTGGCGATAATATCTTCTATGGACACGACTTGCCGAAATTAATGGAAGCTGCTGTTAACAAA
GAAATCGGTGCAACGGTATTTGCTTATCACGTCAATGATCCTGAACGTTATGGTGTCGTGGAGTTTGATAATAACGG
TACTGCAATTAGCCTGGAAGAAAAACCGCTGGAACCAAAAAGTAACTATGCGGTTACTGGGCTTTATTTCTATGACA
ATGATGTTGTAGAAATGGCGAAAAACCTTAAGCCTTCTGCCCGTGGCGAACTGGAAATTACCGATATTAACCGTATT
TATATGGAGCAGGGACGTTTGTCTGTCGCTATGATGGGGCGTGGTTATGCCTGGTTGGATACTGGTACACATCAAAG
TCTTATTGAAGCAAGTAACTTCATTGCCACCATTGAAGAGCGTCAGGGATTAAAGGTATCTTGCCCGGAAGAGATTG
CTTACCGTAAAGGGTTTATTGATGCTGAGCAGGTGAAAGTATTAGCCGAACCGCTGAAGAAAAATGATTATGGTCAG
TATCTGCTAAAAATGATTAAAGGTTATTAATAAAATGAACGTAATTAAAACTGAAATTCCTGATGTGCTGATTTTTG
AACCAAAAGTTTTTGGTGATGAACGTGGCTTCTTTTTTGAGAGTTTTAACCAGAAAGTATTTGAAGAAGCTGTAGGA
CGGAAGGTTGAATTTGTTCAGGATAACCATTCTAAGTCTAAAATAAATGTATTGCGTGGGATGCATTATCAAACACA
AAATACTCAAGGAAAACTGGTTCGGGTAATTTCTGGTTCAGTATATGATGTTGCCGTAGATTTAAGAGAAAAATCAA
AGACATTTGGCAAATGGGTGGGTGTAGAATTATCTGGGAATAATAAAAGACAATTGTGGATCCCCGAAGGTTTTGCC
CATGGTTTTTATGTGTTGGAGGAGAATACCGAATTTGTTTATAAATGTACCGATACTTATAACCCTGCTCATGAACA
CACATTGCTATGGAATGATCCAACTATCAATATAAGTTGGCCAATCATACAAAACTGCAAGCCAATTATTTCTGAAA
AAGATGCTAATGGACATCTTTTTTCACATAAAACCTATTTCTGAAATGCAATATTATGAGTTTAATTAGAAACAGTT
TCTATAATATTGCTGGTTTTGCTGTGCCGACATTAGTTGCAGTCCCTGCTTTGGGGATTCTTGCCAGGCTGCTTGGA
CCGGAGAATTTTGGACTTTTCACACTAGCATTCGCTTTGATAGGATATGCAAGTATTTTCGACGCCGGGATTAGTCG
AGCTGTAATCAGAGAAATCGCTCTTTATCGAGAAAGTGAAAAAGAGCAAATACAAATTATTTCGACAGCAAGTGTAA
TCGTACTATTCTTAGGGGTGGTTGCAGCTTTGTTACTTTATTTTAGTAGTAATAAAGTTGTTGAGTTATTGAATGTT
AGTTCCGTTTATATTGAAACAGCAGTGCGTGCATTCTCTGTTATTTCATTTATAATACCTGTGTATCTGATTAACCA
GATTTGGCTTGGTTATCTGGAAGGGCTAGAAAAATTTGCAAATATAAATGTTCAGAGAATGATTTCTAGCACAAGCT
TGGCTATATTACCAGTGATATTTTGTTATTACAATCCCTCGTTGCTTTATGCTATGTATGGGTTGGTGGTTGGGCGT
GTGATTTCATTTTTGATTAGCGCAATAATTTGTCGAGATATTATTCTTAAAAGTAAACTTTACTTTAATGTGGCAAC
TTGCAATCGTCTTATCTCTTTTGGTGGATGGATAACAGTTAGTAATATCATAAGCCCAATCATGGCATATTTCGACC
GCTTTATCATCTCTCATATTATGGGGGCTTCGAGAATTGCATTTTATACAGCGCCCTCAGAGGGTGTATCAAGGTTA
ATTAATATCCCATATGCTTTGGCAAGAGCTCTATTTCCTAAATTGGCATATAGCAATAATGATGATGAACGAAAAAA
ATTACAACTACAGAGCTACGCAATTATAAGCATTGTATGTCTACCCATAGTTGTTATTGGTGTCATTTTTGCCTCAT
TCATAATGACAACATGGATGGGACCTGATTATGCCTTAGAAGCAGCAACTATCATGAAAATACTTCTTGCTGGTTTT
TTCTTTAACTCTTTAGCGCAAATACCTTATGCATACTTGCAATCTATCGGAAAGTCAAAAATTACCGCATTTGTGCA
TCTCATAGAACTTGCGCCATACTTATTATTATTGTATTACTTCACAATGCATTTCGGCATAATTGGCACGGCAATCG
CTTGGTCACTTAGAACATTTTGTGATTTTGTTATACTACTTTCGATATCGAGAAGAAAATGATTGCGGTTGATATTG
CGCTTGCAACCTACAATGGTGCTAATTTTATTCGGCAACAGATTGAATCTATCCAGAAACAAACTTATAGAAATTGG
CGTCTTATAATAAGTGATGATAACTCGAGTGATGATACTGTTGATATTATTAAGGATATGATGTCTAACGACAGTCG
TATCTATTTGGTAGGAAATAAAAGACAAGGAGGGGTTATTCAGAACTTTAATTATGCTCTTTCACAAACTACATCTG
AAATTGTGTTACTATGTGACCAGGATGACATTTGGCCGGAGGAGCGTCTGGAAATTCTTATAGATAAATTTAAGGCC
TTGCAGCGTAATGATTTTGTTCCGGCAATGATGTTTACTGATTTGAAATTAGTAGACGAAAATAATTGTTTGATTGC
AGAAAGTTTTTATCGAACGAATAATATTAATCCACAAGATAATCTGAAAAATAATAATCTTCTCTGGCGTTCAACGG
TATATGGCTGTACTTGCATCATGAATAAGAAACTTGTTGATATTGCATTGCCTATACCTACATATGCACATATGCAT
GATCAATGGTTGGCATTATTAGCGAAGCAATATGGTAACATTTTTTATTTCGACTATGCGTCTGTTCGTTATAGGCA
ACATTCTACAAATGTTGTTGGTGGTAGAAATAAAACGCCATTTCAAAAATTTAATTCCATACAAAAAAACCTAAAAA
GGATTAATTTGCTAGTGGATAGAACTGTTGCTTTAATTAAATCAAATAACGATTTCTATCCAGGGAATAAAATGGAA
AATAAAATTGATTACTTAAAATTTGGAGTGAATGAAGTATTACCTTATCTTTTTAAAGGAAACAAGAAAGTTTTTTC
ACTTTGTGTATTAATTAGTTTGGCATTACAAAAATGATATATTTATTATTTTTTTTTGCACTGTTTATGATCTGTAC
GTTTTTAACACACAGGCGACAGGCATTATATGTTGTATCTGCGTTAGTATTTCTTTTTTTGGCTTTAACCTATCCAT
CAGGAGGGGACTGGATAGGTTATTTTCTCCATTATGACTGCATGGTTAATGAGCAGTGTAATAATGGTTTTATAATG
TTTGAACCTGGATATGAATTAATTGTTTCCTTATTTGGATATTTGGGATTTCAGACAATTATTATTTTTATAGCCGC
TGTAAATGTAATTCTAATATTAAATTTTGCAAAGCATTTTGAAAACGGAAGTTTTGTTATTGTTGCGATAATGTGCA
TGTTCCTTTGGAGTGTTTATGTTGAGGCGATTAGACAGGCTCTGGCCTTATCTATAGTTATATTTGGGATTCATTCT
CTTTTTTTGGGTAGAAAAAGGAAATTTATAACATTAGTATTATTTGCGTCAACTTTCCATATAACTGCTTTGATTTG
TTTTCTTCTAATGACTCCTCTATTTTCAAAGAAATTAAGCAAGATAATAAGTTATAGCCTATTAATTTTCAGTAGCT
TCTTTTTCGCTTTTTCTGAAACCATATTAAGTGCACTCCTTGCAATTTTGCCAGAAGGATCCATTGCCAGTGAAAAA
TTAAGTTTTTACTTAGCAACCGAGCAATACAGGCCACAGTTATCTATTGGGAGTGGCACTATTCTTGACATTATACT
TATTTTTCTGATATGTGTAAGTTTTAAACGAATAAAGAAATATATGCTCGCTAATTATAATGCTGCAAATGAGATAT
TGCTTATTGGTTGCTGTCTTTATATTTCTTTCGGTATTTTTATCGGGAAAATGATGCCAGTTATGACTCGCATTGGT
TGGTATGGTTTTCCATTTGTTATAGTACTTCTTTATATTAACTTGGGTTATTCAGAATATTTTAAGAGGTATATAAA
TAAAAGAGGGTGTGGGTATAGCAAATTATTAATTGCTTTTTATTTTTTGCTACAAATTTTGCGACCATTAACATATG
ATTATAGCTATTATAATATAATGCACCAGGATACTTTGCTGAATAGGTTTGATGCATTAGATGATGCATCATTAAGA
CAATCAGCGAAGAGAAAATGTTTCGATTTGGGAAAGATAGGATATGGTTTCTTATGTAGTATATAATATCCTGCATT
CATTCGGATAATTTCCTATGGAAGTGTCCTTTGCTCTGTCTGTCCTCATTTGTTGAAATTTTATGTTAATAAGAAGC
TTTAGATAACCACTTAGGAACTGTATGTTTGATCTGTCCAAAAATTATATTATTGTAAGTGCGACGGCGCTGGCTTC
CGGAGGTGCATTAACTATATTAAAGCAATTTATAAAACATGCATCACAAAATTCAAATGACTATATTATGTTTGTAT
CTGCGGGATTGGAGTTGCCGGTCTGTGATAACATCATTTACATAGAAAACACACCAAAAGGATGGTTGAAAAGAATA
TATTGGGATTGGTTCGGTTGTCGGAAGTTTATCTCGGAACATAAGATTAACGTTAAGAAAGTAATTTCTCTACAAAA
TTCCAGTTTGAATGTTCCTTACGAACAGATTATTTACTTGCACCAGCCAATTCCTTTTAGTAAAGTTGATTCTTTTT
TAAAAAATATCACATCCGATAACGTAAAGCTTTTTTTATATAAAAAGTTTTATTCCTATTTTATATTTAAATATGTG
AATGCCAATACAACCATCGTAGTGCAAACGAATTGGATGAAAAAAGGAGTGCTGGAGCAATGTGATAAAATTAGTAC
CGAAAGGGTCCTTGTTATAAAACCTGATATCAAAGCATTTAATAATACTAATTTTGATGTAGATATGGATGTATCTG
CAAAAACACTCTTATATCCAGCGACACCACTTACCTATAAAAATCATTTGGTCATTCTGAAGGCGTTGGTTATTTTA
AAGAAAAAGTATTTTATAGATGATCTGAAATTCCAAGTGACTTTTGAAAAGAATAGGTACAAAAATTTTGATAAGTT
TGTGCAATTAAATAACTTAAGCAAAAACGTTGATTATCTCGGCGTTCTTTCATACTCGAACTTGCAAAAAAAATATA
TGGCGGCATCTTTAATCGTTTTTCCTAGCTATATCGAATCATATGGGTTACCACTCATCGAAGCTGCTAGTTTAGGA
AAAAAAATCATTAGTAGTGATCTTCCTTATGCCCGGGATGTTTTAAAGGATTATAGCGGCGTAGATTTTGTAATTTA
CAATAATGAAGATGGCTGGGCTAAGGCGTTGTTTAATGTTTTAAATGGCAATTCGAAGCTCAATTTTAGGCCTTATG
AAAAAGATAGTCGTTCATCTTGGCCACAGTTCTTCTCTATTTTGAAATAAGGTGTATTATGTTTAATGGTAAAATAT
TGTTAATTACTGGTGGTACGGGGTCTTTCGGTAATGCTGTTCTAAGACGTTTTCTTGACACTGATATCAAAGAAATA
CGTATTTTTTCCCGGGATGAAAAAAAACAAGATGACATGAGGAAAAAATATAATAATCCGAAACTTAAGTTCTATAT
AGGTGATGTTCGCGACTATTCGAGTATCCTCAATGCTTCTCGAGGTGTTGATTTTATTTATCATGCTGCAGCTCTGA
AGCAAGTACCTTCCTGCGAATTCCACCCAATGGAAGCTGTAAAAACGAATGTTTTAGGTACGGAAAACGTACTGGAA
GCGGCAATAGCTAATGGAGTTAGGCGAATTGTATGTTTGAGTACAGATAAAGCTGTATATCCTATCAATGCAATGGG
TATTTCCAAAGCGATGATGGAAAAAGTAATGGTAGCAAAATCGCGCAATGTTGACTGCTCTAAAACGGTTATTTGCG
GTACACGTTATGGCAATGTAATGGCATCTCGTGGTTCAGTTATCCCATTATTTGTCGATCTGATTAAATCAGGTAGA
CCAATGACGATAACAGACCCTAATATGACTCGTTTCATGATGACTCTCGAAGACGCTGTTGATTTGGTTCTTTACGC
ATTTGAACATGGCAATAATGGTGATATTTTTGTCCAAAAGGCACCTGCGGCTACCATCGAAACGTTGGCTATTGCAC
TCAAAGAATTACTTAATGTAAACCAACACCCTGTAAATATAATCGGCACCCGACACGGGGAAAAACTGTACGAAGCG
TTATTGAGCCGAGAGGAAATGATTGCAGCGGAGGATATGGGTGATTATTATCGTGTTCCACCAGATCTCCGCGATTT
GAACTATGGAAAATATGTGGAACATGGTGACCGTCGTATCTCGGAAGTGGAAGATTATAACTCTCATAATACTGATA
GGTTAGATGTTGAGGGAATGAAAAAATTACTGCTAAAACTTCCTTTTATCCGGGCACTTCGGTCTGGTGAAGATTAT
GAGTTGGATTCATAATATGAAAATTTTAGTTACTGGCGCTGCAGGGTTTATCGGTCGAAATTTGGTATTCCGGCTTA
AGGAAGCTGGATATAACGAACTCATTACGATAGATCGTAACTCTTCTTTGGCGGATTTAGAGCAGGGACTTAAGCAG
GCAGATTTTATTTTTCACCTTGCTGGGGTAAATCGTCCCGTGAAGGAGTGTGAATTTGAAGAGGGAAATAGTAATCT
AACTCAACAGATTGTTGATATCCTGAAAAAAAACAATAAAAATACTCCTATCATGCTGAGTTCTTCCATCCAGGCTG
AATGTGATAACGCTTATGGAAAGAGTAAAGCAGCTGCGGAAAAAATCATTCAGCAGTATGGGGAAACGACAAACGCT
AAATATTATATTTATCGCTTGCCGAATGTATTCGGTAAGTGGTGTCGACCAAATTATAACTCCTTTATAGCAACTTT
CTGCCATCGCATTGCAAATGATGAAGCTATTACAATTAATGATCCTTCAGCAGTTGTAAATCTGGTGTATATAGATG
ACTTTTGTTCTGACATATTAAAGCTATTAGAAGGAGCGAACGAAACTGGTTACAGGACATTTGGTCCAATTTATTCT
GTTACTGTTGGTGAAGTGGCACAATTAATTTACCGGTTTAAAGAAAGTCGCCAAACATTAATCACCGAAGATGTAGG
TAATGGATTTACACGTGCATTGTACTCAACATGGTTAAGTTACCTGTCTCCTGAACAGTTTGCGTATACGGTTCCTT
CTTATAGTGATGACAGAGGGGTATTCTGTGAAGTATTGAAAACGAAAAACGCGGGCCAGTTTTCGTTCTTTACTGCG
CATCCAGGAATTACTCGGGGTGGTCATTATCATCATTCCAAAAATGAGAAATTTATTGTCATCCGAGGAAGTGCTTG
TTTCAAATTTGAAAATATTGTCACGAGTGAACGATATGAACTTAATGTTTCCTCTGATGATTTTAAAATTGTTGAAA
CAGTTCCGGGATGGACGCATAACATTACTAATAATGGCTCGGATGAGCTAGTTGTTATGCTTTGGGCAAATGAAATA
TTTAATCGTTCTGAACCAGATACTATAGCGAGAGTTTTATCGTGAAAAAATTGAAAGTCATGTCGGTTGTTGGGACT
CGTCCAGAAATTATTCGACTCTCGCGTGTCCTTGCAAAATTAGATGAATATTGTGACCACCTTATTGTTCATACCGG
GCAAAACTACGATTATGAACTGAATGAAGTTTTTTTCAAAGATTTGGGTGTTCGCAAACCTGATTATTTTCTTAATG
CCGCAGGTAAAAATGCAGCAGAGACTATTGGACAAGTTATCATTAAAGTTGATGAGGTCCTTGAACAGGAAAAACCA
GAAGCCATGTTAGTACTTGGCGATACTAACTCCTGTATTTCAGCAATACCAGCAAAGCGTCGAAGAATTCCGATCTT
CCATATGGAGGCTGGGAATCGTTGTTTTGACCAACGCGTACCGGAAGAAACTAACAGAAAAATAGTTGATCATACCG
CTGATATCAATATGACATATAGTGATATCGCGCGTGAATATCTTCTGGCTGAAGGTGTACCAGCCGATAGAATTATT
AAAACCGGTAGCCCAATGTTTGAAGTACTCACTCATTATATGCCGCAGATTGATGGTTCCGATGTACTTTCTCGCCT
GAATTTAACACCTGGGAATTTCTTTGTGGTAAGTGCCCACAGAGAAGAAAATGTTGATACCCCTAAACAACTTGTGA
AACTGGCGAATATACTTAATACCGTGGCTGAAAAATATGATGTCCCGGTAGTTGTTTCTACTCATCCTCGCACTCGT
AACCGCATCAACGAAAACGGTATTCAATTCCATAAAAATATCTTGCTTCTTAAGCCATTAGGATTTCACGATTACAA
CCATCTGCAAAAAAATGCACGTGCTGTTTTATCGGATAGTGGGACTATTACAGAAGAGTCCTCCATTATGAACTTCC
CTGCACTCAATATACGAGAAGCGCACGAACGCCCGGAAGGCTTCGAAGAAGGGGCAGTAATGATGGTCGGTCTTGAA
TCTGATCGCGTTTTACAGGCATTAGAAATTATTGCAACACAGCCTCGTGGAGAAGTACGCTTACTTCGTCAGGTTAG
TGACTATAGCATGCCAAATGTTTCAGATAAAGTTCTGCGTATTATCCATTCATATACTGACTACGTTAAACGGGTTG
TCTGGAAGCAATACTAATGAAACTTGCATTAATCATTGATGATTATTTGCCCCATAGCACACGCGTTGGGGCTAAAA
TGTTTCATGAGTTAGGCCTTGAATTACTGAGCAGAGGCCATGATGTAACTGTAATTACGCCTGACATCTCATTACAA
GCAATTTATTCTATTAGTATGATTGATGGTATAAAGGTTTGGCGTTTCAAAAGTGGACCTTTAAAGGATGTAGGTAA
GGCTAAACGTGCCATAAATGAAACTCTTTTATCTTTTCGCGCATGGCGCGCATTTAAGCACCTCATTCAACATGATA
CATTTGATGGTATCGTTTATTATTCCCCCTCTATTTTTTGGGGCGACTTGGTTAAAAAAATAAAACAACGATGCCAG
TGCCCAAGCTATCTGATCCTAAGGGATATGTTTCCACAGTGGGTCATTGATGCAGGTATGTTGAAAGCCGGTTCACC
AATTGAAAAATATTTTAGGTATTTTGAAAAAAAGTCATATCAGCAGGCTGGCCGGATAGGGGTAATGTCTGATAAGA
ATCTTGAGATATTTCGCCAGACCAATAAAGGTTATCCGTGTGAAGTTTTACGTAATTGGGCCTCAATGACTCCTGTG
TCTGCCAGCGATGATTATCATTCACTTCGTCAAAAATACGATCTAAAAGATAAAGTCATTTTTTTCTATGGCGGTAA
TATTGGGCATGCTCAGGATATGGCAAACTTAATGCGCCTTGCGCGTAATATGATGCGTTATCATGATGCTCATTTCC
TGTTTATAGGGCAGGGTGATGAAGTTGAGCTGATAAAATCTCTTGCTGCAGAATGGAATTTAACTAATTTCACTCAT
CTACCTTCAGTGAACCAGGAAGAGTTTAAATTAATTTTATCTGAAGTTGATGTCGGCCTGTTCTCCCTTTCATCTCG
CCATTCTTCACATAATTTCCCCGGAAAATTACTAGGGTATATGGTTCAATCAATCCCGATCCTTGGGAGTGTGAATG
GCGGCAATGATTTAATGGATGTAATTAATAAGCACAGAGCCGGTTTCATTCATGTTAATGGTGAAGATGATAAACTG
TTTGAATCTGCACAATTGCTTCTTAGTGATTCAGTTTTAAGAAAACAGCTAGGTCAGAACGCTAATGTGTTGTTAAA
GTCTCAATTTTCGGTTGAATCGGCGGCACATACTATCGAAGTCCGACTGGAGGCTGGAGAATGCGTTTAGTTGATGA
CAATATTCTGGATGAACTTTTTCGCACTGCAGCAAATTCTGAACGTTTGCGCGCTCATTATTTATTGCACGCATCTC
ATCAGGAGAAGGTTCAACGTTTACTTATTGCATTTGTACGCGACAGCTATGTTGAACCCCATTGGCATGAGTTACCG
CATCAGTGGGAAATGTTTGTCGTCATGCAAGGGCAATTAGAAGTTTGTTTGTATGAGCAAAATGGTGAGATCCAAAA
ACAGTTTGTTGTTGGAGACGGTACGGGAATAAGCGTCGTGGAATTTTCCCCAGGAGATATACATAGTGTCAAATGCC
TGTCACCAAAAGCCCTTATGTTGGAGATAAAGGAGGGGCCATTTGACCCACTCAAAGCTAAGGCTTTTTCTAAGTGG
TTATAGGGCGATACACCACCGTTTATTCTTCTATCTTATTCTATACATGCTGGGTTACCATCTTAGCTTCTTCAAGC
CGCGCAACCCCGCGGTGACCACCCCTGACAGGAGTAGCTAGCATTTGACCACCCCTGACAGGATTAGCTAGCATATG
AGCTCGAGGATATCTACTGTGGGTACCCGGGATCCGTGTAGGCTGGAGCTGCTTCGAAGTTCCTATACTTTCTAGAG
AATAGGAACTTCGGAATAGGAACTAAGGAGGATATTCATAT
(example signal sequence for EPA carrier protein)
SEQ ID NO: 10
MKKIWLALAG LVLAFSASA
(example O1A rfb locus nucleotide sequence-O1A-EPA production strain
stGVXN4411 and stLMTB10217)
SEQ ID NO: 11
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGCTAGCGTGAAGATACTTGTTACTAGGGGCGCAGGATTTATTG
GTTCTGCTGTAGTTCGTCACATTATAAATAATACGCAGGATAGTGTTGTTAATGTCGATAAATTAACGTACGCCGGA
AACCTGGAATCACTTGCTGATGTTTCTGACTCTGAACGCTATGTTTTTGAACATGCGGATATTTGCGATGCTGCTGC
AATGGCGCGGATTTTTGCTCAGCATCAGCCGGATGCAGTGATGCACCTGGCTGCTGAAAGCCATGTGGATCGTTCAA
TTACAGGCCCTGCGGCATTTATTGAAACCAATATTGTTGGTACTTATGTCCTTTTGGAAGCGGCTCGCAATTACTGG
TCTGCTCTTGATGGCGACAAGAAAAATAGCTTCCGTTTTCATCATATTTCTACTGACGAAGTCTATGGTGATTTGCC
TCATCCTGACGAAGTAAATAATAAAGAACAATTACCCCTCTTTACTGAGACGACAGCTTACGCGCCTAGTAGTCCTT
ATTCCGCATCAAAAGCATCCAGCGATCATTTAGTCCGCGCGTGGAAACGTACCTATGGTTTACCGACTATTGTGACT
AACTGTTCGAATAACTACGGTCCTTATCACTTTCCGGAAAAATTGATTCCACTAGTAATTCTTAATGCTCTGGAAGG
TAAGGCATTACCTATTTATGGCAAAGGGGATCAAATTCGTGACTGGCTGTATGTTGAAGATCATGCGCGTGCGTTAT
ATACCGTAGTTACTGAAGGTCAAGCGGGTGAAACCTATAACATTGGCGGACACAACGAAAAGAAAAACATCGATGTT
GTGCTGACTATTTGTGATTTGTTGGACGAGATAGTCCCGAAAGAGAAATCTTATCGTGAGCAAATTACTTATGTTGC
TGATCGCCCAGGGCATGATCGCCGTTATGCGATTGATGCTGAGAAGATTGGTCGCGAATTGGGATGGAAACCACAGG
AAACGTTTGAGAGTGGGATTCGTAAAACGGTGGAATGGTATTTGGCTAATGCAAAATGGGTTGATAATGTGAAAAGT
GGTGCCTATCAATCGTGGATTGAACAGAACTATGAGGGCCGCCAGTAATGAATATCCTCCTTTTTGGCAAAACAGGG
CAGGTAGGTTGGGAACTACAGCGTGCTCTGGCACCTCTGGGTAATTTGATTGCTCTTGATGTTCACTCCACTGATTA
CTGTGGTGATTTTAGTAACCCTGAAGGTGTGGCTGAAACAGTCAAAAGAATTCGACCTGATGTTATTGTTAATGCTG
CGGCTCACACCGCAGTAGATAAGGCTGAGTCAGAACCCGAATTTGCACAATTACTCAATGCGACTAGCGTTGAATCA
ATTGCAAAAGCGGCAAATGAAGTTGGGGCTTGGGTAATTCATTACTCAACTGACTACGTATTCCCTGGAAATGGCGA
CACGCCATGGCTGGAGATGGATGCAACCGCACCGCTAAATGTTTACGGTGAAACCAAGTTAGCTGGAGAAAAAGCAT
TACAAGAGCATTGTGCGAAGCACCTAATTTTCCGTACCAGCTGGGTCTATGCAGGTAAAGGAAATAATTTCGCCAAA
ACGATGTTGCGTCTGGCAAAAGAGCGTGAAGAACTAGCCGTTATTAATGATCAGTTTGGTGCGCCAACAGGTGCTGA
ACTGCTGGCTGATTGTACGGCACATGCCATTCGTGTCGCACTGAATAAACCGGATGTCGCAGGCTTGTACCATTTGG
TAGCCAGTGGTACCACAACCTGGTACGATTATGCTGCGCTGGTTTTTGAAGAGGCGCGCAATGCAGGCATTCCTCTT
GCACTCAACAAGCTCAACGCAGTACCAACAACTGCCTATCCTACACCAGCTCGTCGTCCACATAACTCTCGCCTTAA
TACAGAAAAATTTCAGCAGAATTTTGCGCTTGTATTGCCTGACTGGCAGGTTGGTGTGAAACGCATGCTCAACGAAT
TATTTACGACTACAGCAATTTAATAGTTTTTGCATCTTGTTCGTGATGGTGGAGCAAGATGAATTAAAAGGAATGAT
GAAATGAAAACGCGTAAAGGTATTATTTTAGCGGGTGGTTCTGGTACTCGTCTTTATCCTGTGACTATGGTCGTCAG
TAAACAGCTATTACCTATATATGATAAACCGATGATCTATTATCCGCTTTCTACACTGATGTTAGCGGGTATTCGCG
ATATTCTGATTATTAGTACGCCACAGGATACTCCTCGTTTTCAACAACTGCTGGGTGACGGTAGCCAGTGGGGCCTG
AATCTTCAGTACAAAGTGCAACCGAGTCCGGATGGTCTTGCGCAGGCATTTATTATCGGTGAAGAGTTTATTGGTGG
TGATGATTGTGCTTTGGTACTTGGTGATAATATCTTCTACGGTCACGACCTGCCTAAGTTAATGGATGCCGCTGTTA
ACAAAGAAAGTGGTGCAACGGTATTTGCCTATCACGTTAATGATCCTGAACGCTATGGTGTCGTTGAGTTTGATAAA
AACGGTACGGCGATCAGCCTGGAAGAAAAACCGCTACAACCAAAAAGTAATTATGCGGTAACCGGGCTTTATTTTTA
TGATAACGACGTTGTCGAAATGGCGAAAAATCTTAAGCCTTCTGCCCGCGGTGAACTGGAAATTACCGATATTAACC
GTATCTATATGGAACAAGGGCGTTTATCTGTTGCCATGATGGGGCGTGGTTATGCGTGGTTAGACACGGGGACACAT
CAGAGCCTGATTGAGGCAAGCAACTTTATTGCAACAATTGAAGAGCGTCAGGGGCTGAAAGTTTCCTGCCCGGAAGA
AATTGCTTACCGTAAAGGGTTTGTTGATGCTGAGCAGGTGAAAGTATTAGCTGAACCTCTGAAAAAAAATGCTTATG
GTCAGTATCTGCTGAAAATGATTAAAGGTTATTAATAAAATGAACGTAATTAAAACAGAAATTCCTGATGTACTGAT
TTTTGAACCGAAAGTTTTTGGTGATGAGCGTGGTTTCTTTTTTGAGAGCTTTAACCAGAAGGTTTTTGAGGAAGCTG
TAGGCCGCAAAGTTGAATTTGTTCAGGATAACCATTCGAAGTCTAGTAAAGGTGTTTTACGCGGGCTGCATTATCAG
TTGGAACCTTATGCACAAGGAAAATTGGTGCGTTGCGTTGTCGGTGAAGTTTTTGACGTAGCTGTTGATATTCGTAA
ATCGTCATCGACTTTTGGCAAATGGGTTGGGGTGAATTTATCTGCTGAGAATAAGCGGCAATTGTGGATTCCTGAGG
GATTTGCACATGGTTTTTTAGTGCTGAGTGAGACGGCGGAGTTTTTGTATAAGACGACAAATTATTATCATCCTCAG
AGTGATAGAGGAATAAAATGGGATGATCCAAGCATCAATATTTCATGGCCAGTCGATTCACAAGTGCTGCTATCAGC
TAAAGATAATAAGCATCCTCCATTAACAAAGATTGAAATGTATAGTTAAGATCACGATAAATCTTGGAAGGGTTGCA
AAATTGAATAAAATAGTGAGCAAAAGTGAAATAAGGAACGTAATCCACAATGCTGGCTATATGATGATTACTCAGAT
AGCTTTATATGTTGCACCATTATTTATACTGAGTTATCTGTTAAAAACACTGGGGGTTGCACAGTTTGGTAATTATG
CCTTAATACTATCAATCGTTGCATATTTACAGATTATAACGGATTATGGTTTTTCTTTTAGTGCAAGTCGTGCGATC
TCACAGAATAGAGAGGACAAAGAATATATATCAAAAATTTATCTGTCAACTATGACTATCAAGTTGGCGATATGCGC
TTTCTTATTCTTATTGCTCATGCTATTTTTAAATCTTTTGCCTGTGCAAGCTGAATTAAAACAAGGAATATTATATG
GATATCTTCTTGTAATAGGAAATACTTTCCAACCACAATGGTTTTTCCAAGGTATCGAAAAATTAAAAATCATAGCC
CTTTCTAATGTTATATCAAGATGCGCCGCGTGTTTACTTGTATTTATCTATGTGAGGAATAGCGAGGATTTACAAAA
AGCACTTTTAGTACAGTCACTTCCATTAGTAATTTCTGCGATTGGATTAAATATATTTATATTGAAATATATCAATA
TTATTTTTCCGGAAAAAAAATTATTTAAGGTAATTTTAAAAGAAGGTAAGGATTTTTTTCTTGCATCACTTTATTCT
GTTATTCTCAATAATAGTGGCATTTTTCTATTAGGGATTTTTACTAATCCTGTTATTGTTGGTGTATATGCCGCCGC
TGAAAAGATAGTCAAGGCCGTATTGTCGCTATTTACACCACTGACGCAAGCTATATATCCTTATAATTGTCGTAAGT
TTTCACTATCCGTATTTGACGGCATTGAGGCAGCAAAAAAAACTGGTATACCAATTATAATTTTAGCATTTATAGCT
GCTGTTATCGTTGCAATTACCTTACCTGTTGCAATCGACTATCTTAATTTTCCAAAAGAAACAATTTTTGTAGGTCA
AATATTAAGTGCATGGATCTTTTTTGGTGTTCTTAATAATGTATTCGGCATTCAGATATTGAGTGCATCAGGAAGAA
GTAAAATATATAGTAGGATGGTATTCGTATCAGCGCTTATAACATTACTTTTGATTACTCTATTATTGCAGTTTTGT
AACGCCACTGGAGTGGCATGTGCAATATTATTGGGTGAAATGTTCTTATCAATATTGTTACTTAAGCGATATAAAAA
AATAATTTAAGGAATAGTTATGAAGAAGTTATTATTAGTGTTCGGTACTAGGCCTGAAGCAATAAAGATGGCCTCTA
TCATTGAATTATTAAAAAAAGATTGTAGATTCGAATATAAAATATGTGTGACAGGCCAACATAAAGAGATGCTTGAT
CAAGTTATGCAAGTATTTGATGTTAAACCTGATTATAATTTACGGATTATGCAGCCTGGGCAAACATTAGTATCTAT
AGCAACAAATATACTCTCACGGTTAAGTGAAGTTTTAATTATAGAAAAGCCAGATATTATACTTGTGCATGGGGATA
CAACGACTACCCTTGCTGCTACTTTAGCTGGGTATTACCACCAAATAAAAGTTTGTCATGTGGAAGCAGGATTAAGA
ACAGGGGATATTTACTCTCCTTGGCCTGAAGAGGGCAATCGTAAAGTTACAGGGGCATTAGCATGTATTCATTTCGC
CCCAACAGAGAGATCAAAAGATAATCTCCTGAGGGAGGGGGTCAAAGTAAATAATATATTTGTAACGGGTAATACCG
TCATCGACTCTTTATTTATTGCAAAAGATATCATAGATAATGACCCTAATATAAAGAACGCTTTACATAATAAATTT
AATTTTCTTGATAAAAGCCGACGAGTAGTACTTATAACAGGTCATCGAAGAGAAAATTTCGGGAAAGGTTTTGAAGA
TATATGCTTTGCAATAAAGGAATTAGCTTTCATTTATCCTAATGTAGATTTTATTTATCCGGTGCATCTTAATCCCA
ATGTAATGGAACCAGTACATCGTATATTAGATAATATATGTAATATTTACCTTATTGAGCCCTTGGATTATTTGCCT
TTTGTTTATTTAATGAATGAGTCATATTTAATATTGACTGATTCAGGGGGGATACAAGAAGAAGCGCCTTCGTTAGG
TAAACCGGTTTTGGTTATGCGTGATACTACTGAACGCCCTGAGGCGGTTGAGGCTGGTACTGTTGTATTAGTGGGGA
CTTCTAAGATAAAAATAGTAAATAAAGTAACGGAGCTATTAAACAATGCTGATATCTACAATGCTATGTCTCTGTTA
CATAATCCATATGGCGATGGAACAGCTGCTCAAAAAATTCTTAATGTGCTCGCCCAAGAGCTAATTTAATTTAAGCT
AAAAATATGTTATTAATTATTGCTGATTATCCAAACGAAATGAATATGCGCGAGGGAGCTATGCAACGAATAGATGC
GATAGACTCTCTCATTCGAGATCGCAAGCGAGTGTATTTGAATATTTCATTCAAAAAGCATCTAGTTCGCTCAAATA
GTTCCTTTAATAATGTTATAGTTGAAAATCTAAATGCAATTATTCACAGAAACATCATAAAACAGTACATGCAAAAA
TCAACAACTATATATGTTCATTCTGTTTATAATTTATTAAAGGTTATAACGCTCATTGATCTAAAAAAAACAATTCT
TGATATACATGGTGTTGTACCGGAAGAACTTTTGGCAGATAATAAAAAATTACTTAGTAAAGTATATAACATGGTGG
AAAAAAAAGGTGTCCTTGGATGCAAAAAATTAATACACGTCAGTACAGAAATGCAAAAACACTATGAAGCAAAATAT
GGAGTAAACTTGGCTGAAAGGTCAATAGTGCTCCCGATTTTTGAATATAAAAATATAACCCAATCGCAAAACAAATG
GACAGAAAATAAAATACGAAGTATCTATCTTGGAGGATTACAAACATGGCAAAATATTGATAAAATGATTCAAGTTT
GTGATGACACAGTGATAAACAATGAAGCAGGTAAGTATGAATTCAACTTTTTCATCCCACAGAGTAACTTGGAAGGG
TTTATAGATAAATATTCGTTAAAATTACATAATATCAATGCTAATGCATCTACGCTATCACGTGATGAAGTAATTCC
CTTTCTAAAAGAATGTCATATTGGTTTTGTATTGCGCGATGATATAATAGTAAACAGAGTTGCGTGCCCTACAAAAT
TGGTTGAATATTTAGAGTGTGGTGTCGTTCCAGTTGTGCTCTCCCCACTTATAGGTGATTTTTATTCGATGGGATAT
CAATACATTACTACAGAGGAAATGGCTAACAGAAGTATAAGTTTGTTGGATCTTGAAAAAATGGCTGCACATAATTT
ACAAATTTTGACTTCTTATCAGAAGAGAACCTACAAGGCACAGAAAGAACTTATTGCTCAACTGTGCTGAATTTTTT
ACATATATAAAATTATGTAAGCATATCGCGGGTCAGGTAATTGTATGCGTATCAAATATAAAGATAACGGTTATATA
TTATGTTTTCTATTATGTTTCATTTTGAGCTACTTAGTTTTACTCAAATCTGACTACTTTCCTGCTGATTTTCTGCC
ATATACAGAAATATACGATGGGACATACGGAGAAATCAATAATATTGAGCCTGCCTTTTTATATTTAACACGGTTGT
TTCATTATTTAAATTTCCCCTATATATTTTTTGCAATGTTAGTTTGTGCCTTATGTTTAAGTTGGAAAATAAAATAT
GCAAGAAAAATAATTAAAGATAGTTATATATATTTGTTCTTGTATGTATATGTATCATTTTATGTGTTTTTGCATGA
AATGACTCAATTGCGCATAGCAATTGCAGTCACTATGTGCTATGTGTCGGTTTATTATTACTTTTATAAAAATTGTA
TTAAACATGCACTGCCATGGATGGTGTTGGCTATTTTGTTTCATTACAGCGCCTTGCTTTTATTTATGTCATTATTT
ATATACAGTTATAGGAGGTTATTAATAGTAATTATAGGGTTTGTAATATGTATGAGCTTTTTAAACGTGTATGCAGA
TACAATTGCACTATATTTGCCAAATGAAAAAATAGTAAATTATTTATATAGTATTTCATCATCATTAGACAATAGAA
ATGATTTGGCAATATTCAACCTGAATAATATAATATTTTTATCAATATTTATTTTGATCTTTTATCTTAGCCGATAT
ATAAAATTAAATGATAATGAGGCGAAGTTTATTAAGTATGTGCAATGTTCAGGAATATTAGCCTTTTGTATTTTCTT
TCTGGCTAGTGGAGTCCCGGTCATTGCTTATCGAACTGCAGAGTTGCTGCGAATATTTTATCCGATGGCTTTAGTAT
TAATCCTTTCGCATATAAAAAATAATAATATGCGTTATTTTATTGCAGTCATTATAGTTATCCTTTCAGGCTTAATG
TTGTTTATAACACTAAGGGCTGTATCAATAGTTGGTCAAGGATTATAAAATGAATGTTGCTATTTTGTTGTCTACGT
ATAATGGCGAAAAATATTTAGAGGAACAACTGGATTCATTGCTGCTTCAAAGTTATCAGGATTTTGTAGTGTATATC
CGTGATGACGGATCATCTGATAGAACTGTAAATATAATAAACCAATACGTAATGAAAGATAACAGATTTATTAACGT
GGGTAATTCAGAAAATCTTGGTTGTGCTGCTTCGTTTATTAATTTATTAAGAAATGCTTCAGCCGATATTTATATGT
TTTGTGACCAAGATGATTATTGGCTTCCGAATAAATTACAGCGTGCTGTGGATTATTTTTCGGCTATTGATCCTTTA
CAACCTACCTTGTATCATTGCGATCTAAGCGTTGTTGATGAAAAACTTAATATTATACAAAATTCATTTTTGCAGCA
TCAGAAAATGTCAGCGTATGATTCAATGAGAAAAAATAATCTTTTCATACAAAATTTTGTTGTTGGTTGTTCATGTG
CTGTTAATGCTTCACTTGCGGAATTTGTTCTTTCGCGAATTGGAGAGCAGCATGTAAAAATGATAGCTATGCATGAC
TGGTGGTTAGCCGTGACTGCAAAACTTTTTGGTCGAATCCATTTTGATAATACTCAAACGATTCTTTATCGACAACA
TCAGGGCAATGTATTAGGTGCAAAATCATCAGGTATGATGCGTTTTATTCGATTAGGATTAAATGGGCAAGGGATTT
CGCGAGTAGTATCTTTTAGAAAAAAAGTTTGTGCGCAAAATAAGCTTCTTTTAGATGTCTATGATAAAGATTTAAAT
CTTGAGCAAAAAAAATCTATCAGGCTTGTAATTGAGGGCCTTAAAGAGAACTCTTCAATTGCTGACCTTTTAAAATG
TTTCTATCATGGTAGCTATATGCAAGGTTTTAAACGTAATCTTGCCTTAATATATTCAGTTCTTTACACAAAAAAAA
GAAGATAGTGTATCCTTATGAAAAAAATTGCTATTATCGGTACTGTTGGCATACCAGCATCATATGGCGGATTTGAA
ACATTAGTTGAAAATTTAACAAGATACAATTCCTCGGGAGTTGAATATAATGTTTTTTGTTCATCGTTTCACTACAA
ATCCCACCAAAAAAAACATAATGGGGCCCGTTTAATTTATATTCCGCTTAAAGCCAATGGATGGCAGAGCATTGCGT
ATGACATAATTTCGTTAGCATATTCTATTTTTTTGAAGCCTGATGTGATTCTGATTTTAGGGGTTTCTGGTTGTTCA
TTTTTGCCTTTCTTCAAACTCTTAACACGCGCTAAGTTTATTACTAATATTGATGGCCTGGAATGGCGAAGAGATAA
ATGGAATTCAAAAGTGAAACGTTTCTTAAAATTTTCAGAAAAAATCGCAGTTCAATATTCGGATGTCGTTATTACGG
ATAATGAGGCAATTTCTGAGTACGTTTTTAACGAGTATAATAAAGATAGCCGAGTTATTGCCTATGGAGGGGATCAT
GCATGGTTAAATACTGAGGATGTATTTACAACAAGAAATTATAAAAGCGATTACTACCTTTCTGTATGTCGTATCGA
ACCCGAAAACAATGTAGAATTAATTTTAAAAACATTTTCAAAGCTAAAATATAAAATAAAATTTATTGGAAATTGGA
ATGGCAGCGAGTTTGGAAAGAAACTTAGGCTGCATTATTCTAACTATCCAAATATTGAAATGATTGATCCGATTTAT
GATCTTCAACAATTATTTCACTTACGAAATAATTGCATAGGATATATACATGGTCATTCGGCTGGAGGAACAAACCC
TTCTTTAGTCGAGGCAATGCATTTTAGTAAACCTATATTTGCATATGATTGTAAGTTTAATAGGTACACTACTGAAA
ATGAAGCATGTTATTTTTCTAATGAATCTGACCTCGCAGAGAAAATCATAATGCATTGTGAGCTATCATTAGGTGTC
TCTGGCACGAAAATGAAAGAAATTGCTAACCAGAAATACACTTGGAGACGAATAGCAGAAATGTATGAGGATTGCTA
TTAACTCTGTTAAACTTCAAATCTTTTACAATATATGGCATGACTATAAGCGCATTAATTGTTTTTCAAGCCGCTCT
CGCGGTGACCACCCCCTGACAGGGGATCCGTGTAGGCTGGAGCTGCTTCGAAGTTCCTATACTTTCTAGAGAATAGG
AACTTCGGAATAGGAACTAAGGAGGATATTCATATGGATAAAGCCGTAAGCATATAAGCATGGATAAGCTATTTATA
CTTTAATAAGTACTTTGTATACTTATTTGCGAACATTCCAGGCCGCGAGCATTCAGCGCGGTGATCACACCTGACAG
GAGTATGTAATGTCCAAGCAACAGATCGGCGTAGTCGGTATGGCAGTGATGGGACGCAACCTTGCGCTCAACATCGA
AAGCCGTGGTTATACCGTCTCTATTTTCAACCGTTCCCGTGAGAAGACGGAAGAAGTGATTGCCGAAAATCCAGGCA
AGAAACTGGTTCCTTACTATACGGTGAAAGAGTTTGTCGAATCTCTGGAAACGCCTCGTCGCATCCTGTTAATGGTG
AAAGCAGGTGCAGGCACGGATGCTGCTATTGATTCCCTCAAACCATATCTCGATAAAGGAGACATCATCATTGATGG
TGGTAACACCTTCTTCCAGGACACTATTCGTCGTAATCGTGAGCTTTCAGCAGAGGGCTTTAACTTCATCGGTACCG
GTGTTTCTGGCGGTGAAGAGGGGGCGCTGAAAGGTCCTTCTATTATGCCTGGTGGCCAGAAAGAAGCCTATGAATTG
GTAGCACCGATCCTGACCAAAATCGCCGCCGTAGCTGAAGACGGTGAACCATGCGTTACCTATATTGGTGCCGATGG
CGCAGGTCACTATGTGAAGATGGTTCACAACGGTATTGAATACGGCGATATGCAGCTGATTGCTGAAGCCTATTCTC
TGCTTAAAGGTGGCCTGAACCTCACCAACGAAGAACTGGCGCAGACCTTTACCGAGTGGAATAACGGTGAACTGAGC
AGTTACCTGATCGACATCACCAAAGATATCTTCACCAAAAAAGATGAAGACGGTAACTACCTGGTTGATGTGATCCT
GGATGAAGCGGCTAACAAAGGTACCGGTAAATGGACCAGCCAGAGCGCGCTGGATCTCGGCGAACCGCTGTCGCTGA
TTACCGAGTCTGTGTTTGCACGTTATATCTCTTCTCTGAAAGATCAGCGTGTTGCCGCATCTAAAGTTCTCTCTGGT
CCGCAAGCACAGCCAGCAGGCGACAAGGCTGAGTTCATCGAAAAAGTTCGTCGTGCGCTGTATCTGGGCAAAATCGT
TTCTTACGCCCAGGGCTTCTCTCAGCTGCGTGCTGCGTCTGAAGAGTACAACTGGGATCTGAACTACGGCGAAATCG
CGAAGATTTTCCGTGCTGGCTGCATCATCCGTGCGCAGTTCCTGCAGAAAATCACCGATGCTTATGCCGAAAATCCA
CAGATCGCTAACCTGTTGCTGGCTCCGTACTTCAAGCAAATTGCCGATGACTACCAGCAGGCGCTGCGTGATGTCGT
TGCTTATGCAGTACAGAACGGTATTCCGGTTCCGACCTTCTCCGCAGCGGTTGCCTATTACGACAGCTACCGTGCTG
CTGTTCTGCCTGCGAACCTGATCCAGGCACAGCGTGACTATTTTGGTGCGCATACTTATAAGCGTATCGATAAAGAA
GGTGTGTTCCATACCGAATGGCTGGATTAA
(example O2 rfb locus nucleotide sequence-O2-EPA production strain
stGVXN4906)
SEQ ID NO: 12
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGTGAAAATACTTGTTACTGGTGGCGCAGGATTTATTGGTTCAG
CTGTAGTTCGTCACATTATAAATAATACGCAGGATAGTGTTGTTAATGTCGATAAATTAACGTACGCCGGAAACCGG
GAATCACTTGCTGATGTTTCTGATTCTGAACGCTATGTTTTTGAACATGCGGATATTTGCGATGCACCTGCAATGGC
ACGGATTTTTGCTCAGCATCAGCCGGATGCAGTGATGCACCTGGCTGCTGAAAGCCATGTTGACCGTTCAATTACAG
GCCCTGCGGCATTTATTGAAACCAATATTGTTGGTACTTATGTCCTTTTGGAAGCCGCTCGCAATTACTGGTCTGCT
CTTGATAGCGACAAGAAAAATAGCTTCCGTTTTCATCATATTTCTACTGACGAAGTCTATGGTGATTTGCCTCATCC
AGATGAAGTAAATAATACAGAAGAATTACCCTTATTTACTGAGACGACAGCTTACGCGCCAAGCAGCCCTTATTCCG
CATCCAAAGCATCCAGCGATCATTTAGTCCGCGCATGGAAACGTACGTATGGTTTACCGACCATTGTGACTAATTGC
TCGAACAACTATGGTCCGTATCACTTCCCGGAAAAGCTTATTCCATTGGTTATTCTTAATGCACTGGAAGGTAAGGC
ATTACCTATTTATGGCAAAGGGGATCAAATTCGCGACTGGTTGTATGTAGAGGATCATGCTCGTGCGTTATATACCG
TCGTAACCGAAGGTAAAGCGGGTGAAACTTATAACATTGGCGGACACAACGAAAAGAAAAACATCGATGTTGTGCTG
ACTATTTGTGATTTGTTGGATGAGATTGTACCGAAAGAGAAATCTTATCGTGAGCAAATTACTTATGTTGCTGATCG
CCCAGGGCATGATCGCCGTTATGCAATTGATGCCGATAAAATTAGCCGCGAATTGGGCTGGAAACCACAGGAAACGT
TTGAGAGCGGGATTCGCAAAACGGTGGAATGGTATCTGGCTAATACAAATTGGGTTGAGAATGTGAAAAGCGGTGCT
TATCAGTCATGGATCGAACAAAACTATGAGGGCCGTCAGTAATGAATATCCTGCTTTTCGGCAAAACAGGGCAGGTG
GGTTGGGAACTGCAGCGTGCTCTGGCGCCGCTGGGTAATCTGATCGCTCTTGATGTTCACTCCACTAATTATTGTGG
AGATTTCAGCAACCCCGAAGGTGTGGCAGAAACCGTCAAAAAAATTCGTCCTGACGTTATTGTTAATGCTGCTGCTC
ACACTGCAGTAGATAAAGCAGAATCAGAACCGGATTTCGCACAATTACTTAACGCGACAAGCGTCGAAGCGATTGCA
AAAGCTGCTAATGAAGTCGGGGCCTGGGTTATACACTACTCTACTGATTATGTTTTCCCAGGCAGTGGTGACGCGCC
ATGGCTGGAAACGGATGCAACAGCACCGCTAAATGTTTACGGTGAAACAAAATTAGCTGGGGAAAAGGCATTACAAG
AACATTGCGCAAAGCATCTTATTTTCCGTACCAGCTGGGTATACGCTGGTAAAGGAAATAACTTTGCTAAAACGATG
TTGCGTTTGGCAAAAGAACGCGAAGAACTGGCTGTGATAAACGATCAGTTTGGCGCACCAACAGGTGCTGAATTGCT
GGCTGATTGCACCGCTCATGCCATTCGCGTGGCATTAAAAAAACCAGAAGTCGCTGGCTTGTACCATCTGGTAGCAA
GTGGCACAACAACCTGGCACGATTATGCTGCGCTGGTTTTTGAAGAGGCGCGCAAAGCAGGGATTAATCTTGCACTT
AACAAACTTAACGCCGTGCCAACAACGGCCTATCCCACACCAGCCCGTCGACCCCATAACTCTCGCCTCAATACAGA
AAAGTTTCAGCAGAACTTTGCGCTTGTCTTGCCTGACTGGCAGGTGGGCGTGAAACGTATGCTCAACGAATTATTTA
CGACTACGGCAATTTAACAAATTTTTGCATCTCGCTCATGATGCCAGAGCGGGATGAATTAAAAGGAATGGTGAAAT
GAAAACGCGTAAAGGTATTATTCTGGCTGGTGGTTCCGGCACTCGTCTTTATCCTGTGACGATGGCAGTGAGTAAAC
AATTGCTGCCGATTTATGATAAGCCGATGATTTATTATCCGCTTTCAACGCTTATGTTAGCGGGTATTCGCGATATT
CTTATTATTAGTACGCCACAGGATACACCGCGTTTCCAACAATTATTGGGGGACGGGAGCCAGTGGGGTCTTAATCT
ACAGTATAAAGTACAACCGAGTCCGGATGGCCTGGCGCAAGCGTTTATTATTGGCGAAGACTTTATTGGTGGTGATG
ATTGTGCACTCGTACTTGGCGATAATATCTTCTATGGACACGACTTGCCGAAATTGATGGAAGCTGCTGTTAACAAA
GAAAGCGGTGCAACGGTATTTGCTTATCACGTTAATGATCCTGAACGCTATGGTGTCGTGGAGTTTGATAATAACGG
TACGGCAATTAGCCTGGAAGAAAAACCGCTGGAGCCAAAAAGCAACTATGCGGTTACTGGGCTTTATTTCTATGACA
ATGACGTTGTGGAAATGGCTAAAAACCTTAAGCCTTCTGCCCGTGGCGAACTGGAAATTACCGATATTAACCGTATT
TATATGGAACAAGGACGTTTGTCTGTAGCCATGATGGGGCGTGGCTATGCATGGTTGGATACAGGGACGCATCAAAG
CCTTATTGAAGCAAGTAACTTCATTGCAACAATTGAAGAGCGTCAGGGATTAAAGGTATCTTGCCCGGAAGAGATTG
CTTACCGTAAAGGGTTTATTGATGCCGAGCAGGTGAAAGTATTAGCCGAACCGCTTATCAAGAATCAATATGGTCAA
TATTTGCTGAAAATGATCAGCGAATAGTATATGGGAACTCAATGATGGATATTAAATTAATCTCTTTGCAAAAACAT
GGGGATGAGCGCGGTGCATTAATTGCTCTTGAAGAGCAACGAAATATACCTTTCGAAGTCAAAAGAATATATTACAT
ACTTGAGACTCTTAATGGAGTAAGACGCGGATTTCATGCGCACAAGGTTACTCGTCAGTTAGCTATTGTAGTCAAGG
GAGCTTGTAAATTTCATCTGGATAATGGTAAAGAAACAAAGCAGGTGGAACTTAATGATCCAACAATTGCGTTGCTG
ATAGAACCCTATATATGGCATGAAATGTATGATTTTAGTGATGATTGTGTGCTGCTTGTAATTGCGGATGATTTCTA
TAAAGAGTCTGATTATATCCGCAATTATGATGATTTTATTAGAAGAGTAAATTCAATTGAGAATTCATAAGCTAAGT
GACGTCCAGACAACATCAATTGGTGATGGAACAACTATCTGGCAGTTTGTTGTGATACTAAAAGGTGCTGTAATTGG
TAATAATTGCAACATCTGTGCAAATACCTTAATTGAAAATAACGTTGTAATTGGTAACAATGTCACAGTCAAAAGCG
GTGTGTATATTTGGGATGGCGTTAAAATAGAGGATAATGTTTTTATTGGTCCTTGTGTAGCATTTACAAATGATAAG
TATCCTCGCTCTAAAGTCTATCCTGATGAATTTTTGCAAACAATAATACGCAAAGGAGCATCAATAGGTGCTAACGC
AACCATCCTGCCAGGAATTGAAATTGGTGAAAAAGCAATCGTTGGTGCGGGGAGTGTTGTAACCAAAAATGTACCGC
CATGCGCAATAGTAGTAGGTAATCCAGCTCGATTTATTAAATGGGTAGAGGATAATGAATAAAATTGATTTTTTAGA
TCTTTTTGCAATTAACCAGCGACAGCACAAAGAATTAGTCTCTGCGTTTAGTAGGGTGCTAGATTCTGGTTGGTATA
TCATGGGCGAAGAACTTGAGCAGTTCGAGAAAGAGTTCGCAGAATACTGTGGAGTTAAGTATTGCATTGGTGTAGCA
AATGGCCTTGATGCGTTGATACTAGTATTGAGGGCATGGAAAGAACTTGGCTATCTTGAAGACGGTGACGAGGTATT
AGTACCGGCAAATACATATATTGCTTCTATTCTTGCTATAACAGAGAACAAACTTGTTCCTGTTCTTGTTGAACCAG
ATATAGAAACTTATAATATTAATCCTGCTTTAATTGAAAATTACATTACGGAAAAAACTAAAGCAATATTACCGGTT
CACTTATATGGTCTATTGTGCAATATGCCAGAAATTAGTGCAATCGCCAGAAAATATAATCTGTTGATTCTTGAAGA
TTGTGCACAAGCACATGGTGCAATACGTGATGGTCGCAAAGCTGGAGCTTGGGGGGATGCTGCAGGATTTAGTTTTT
ATCCAGGAAAAAACCTTGGAGCTTTGGGGGATGCGGGAGCTGTTACTACAAATAATGCAGAATTATCCTCAACTATA
AAAGCTTTGCGAAATTATGGGTCACATAAGAAATATGAAAATATTTATCAGGGATTGAATAGTCGATTGGATGAACT
GCAAGCAGCCTTATTGCGTGTAAAAATCCATACATTACCGGAAGATACTGCGATTCGGCAAAGGATTGCTGAAAAAT
ATATTCGTGAAATAAAAAACCCTGCGATTACGTTACCAGTGTACGAAGGCCAAGGTGCGCATGTTTGGCATTTATTT
GTAGTAAGAATCGCTAATCGTGAAAAATTCCAGTCATACTTATTAGAGAAGGGTATCAAAACCTTAATTCACTATCC
ATTACCACCCCATAAGCAGCAAGCATATCAAAATATGTCTAGCCTTAGCCTTCCAATTACTGAGCAAATTCATGATG
AAGTCATTTCTTTACCTATAAGTCCGGTAATGAGTGAAGATGATGTCAATTATGTAATCAAAATGGTCAATGATTAC
AAGTAATGAAAAAATTTCTTCAGGTAACTATATTATCCGCTATCTATACATTCATTAAAATGATTGCGGGTTTTATC
ATCGGTAAGGTAGTAGCAATTTATACAGGGCCATCAGGGGTAGCAATGCTTGGCCAAGTGCAAAGTTTAATCACAAT
AGTTGCAGGTACTACCTCTGCACCTGTAAGCACAGGCCTTGTTCGATATACTGCGGAAAATTGGCAAGAAGGACAAG
AAGCATGCGCGCCATGGTGGCGCGCATGCTTAAGGGTTACTCTGTTTTTATTCTTGCTTATTATTCCCGTTGTTATT
ATATTGTCGAAAAATATTAGTGAGTTACTTTTTAGCGATGGACAATACACATGGTTAATCATTTTCGCATGTTGTAT
ATTGCCATTCTCCATTATAAATACATTGATCGCTTCAGTTTTAAATGGTCAACAATTTTATAAGCAATATATATTGG
TTGGGATGTTTTCTGTATTCATTTCTACTATGTTTATGATTTTGTTGATTGTAGCTTATAATCTTAAAGGTGCATTG
ATTGCCACAGCTATAAATAGTGCTATTGCTGGTCTTGTATTGGTTTTATTTTGTCTCAATAAATCTTGGTTTAGATT
TAAATATTGGTGGGGTAAAACGGATAAAGACAAAATTATAAAAATTATTCATTATACTCTGATGGCTCTGGTTTCTG
TTATCTCCATGCCTACAGCATTGATGTGTATTAGAAAAATATTGATTGCTAAAACTGGTTGGGAGGATGCAGGGCAA
TGGCAGGCCGTATGGAAGATATCTGAGGTTTATCTTGGTGTTGTGACAATTGCTTTGTCAACATATTTCTTACCAAG
ATTGACAATTATAAAAACAAGTTTCCTTATAAAAAAAGAAGTAAATAGTACTATATTATACATAATATCTATTACTT
CATTCATGGCGTTGAGTATCTATTTATTCCGCGATTTGGTAATAACAGTTTTATTTACTGAACAGTTTCGCTCAGCT
CGTGAATTATTTTTATTACAACTTATAGGGGATGTAATAAAAATTGCTGGGTTTCTTTATGCATACCCTCTTCAAAG
TCAGGGGCATACTAAACTATTCATCAGTTCAGAAGTGATTTTTTCTATGCTCTTTATCATTACCACCTATATTTTTG
TTGTAAATTATGGAGTACATGGTGCTAACATAAGTTATGTCATTACATATAGTTTATATTTTGTGTTTGCATTTGTG
TTTACTAATTTTATTAATGTTAGAAGAAATAATTAAAAACAGAGGTTGAATTTTGAAAATAATTATACCTGTCTTAG
GATTTGGCAGGGCTGGTGGTGAAAGAGTTCTTTCTAAGCTGGCAACTGAATTGATGAATTATGGACATGATGTAAGT
TTTGTTGTTCCAGATAATAGAACTAATCCATATTATGCTACCACAGCAAAAATTGTCACGAGTAAATCTAGTCAAAA
CCGTGTAAAAATATTGAGAATCATTAAAAATTACTATAATCTGTGGCGTAAATGCATAGAGTTAAATCCTGATGCTG
TAGTTGCTAGTTTTCATTTGACTGCCTATCTTGTCGCATTATTACCAATCACCCGTCGTAAGAAATATTATTATATT
CAGGCGTATGAAGTTAATTTTTTTGATAATATAATATGGAAATTAATAGCGGGTTTAACATATTATTTACCGCTTAA
AAAAATACTAAATAGTCCTAATTTGCTTCCTCATAAACATGATGATTTTATAGGAGTAGTTCCTGCAGGAGTAGATT
TAAACGTTTTCTATCCGAAACCATCAAATAGGTTATTAAATGGTCACACATCAATAGGGATTATTGGTAGAAAAGAG
AAGCACAAAGGAACTAGCGAAATTATTTCAGTATTGTGTTCACTGGAAAATAAAGCTGGAATTATAATCAATATTGC
GATCTATCTTGAAGAAGTTGATAAGCAGCGTTTAATCGCTGCCGGGTTTCAGGTTAATTTTTTTCCGATTACTTCTG
ATTTAGAATTGGCATCCTTTTATCGAAGCAATGACATCATGATTGCTGTTGGGTTAATTGAAGATGGCGCTTTCCAT
TATCCTTGTGCTGAATCAATGGCTTGTGGTTGTCTTGTTATTTCAAATTATGCGCCACTTACTGAAACTAACAGTGT
ACTTAAATTAGTCAAGTTTGATGCTTGCAAACTTGGTGAAGCAATTAATCTTTGTCTCAATCTTGACCTAGAAGAAA
AAAGCAAAGAAATCCAATCTAATATTTCTGTGTTGAATAAATATGACTGGAAAATTGTTGGTGAAACTTTCAATAGT
TTATTGTTAGATGCAAATAAATAGTATACGTTGATGGGGAAAATATGAATATTGTTAAAACTGATATTCCAGATCTG
ATCGTTCTTGAACCAAAAGTGTTTAGTGATGAACGCGGCTTTTTTATGGAGAGTTATAATCAGATTGAATTTGAGAA
GGCAATAGGAAGGCACGTAAATTTTGTTCAGGATAATCATTCAAAATCTAGTAAAGGCGTACTACGTGGGTTGCATT
ATCAATTAGCACCGTATGCACAGGCTAAATTAGTTCGATGTGTTGTAGGTCAGGTATTTGATGTTGCTGTTGATCTT
AGAAAAAATTCACCAACGTTCAAAAAATGGTTTGGAATAACCCTTTCCGCAGAAAATAAACGACAATTATGGATACC
CGAAGGATTTGCTCATGGTTTCTTGGTGACCAGTGATGAAGCTGAGTTCATTTATAAGACAACTAACTACTATGCTC
CTGGTCATCAGCAAGCAATTATTTACAATGATCCTATTTTAAACATCGATTGGCCTTTCTGCAGTAGTGCTCTGTCA
TTATCACAAAAAGATCAAGAAGCAAAATTATTTTCAGAATTATTGGACAGTGAACTGTTCTAATAAAGTGTGCCACC
TTATCCGTCTGAAGGATAGGTGGTTGCTTATATTTTTTTGAGTATGTTTGTATAATGACAGAAAATAGTCCGAAATA
TAAACACGATAAAAGCTTAATAAGTTTTATCTACTTATTTTTTATATTTACACTTATTGTAGGCTTTATTATCGCAA
ATACCCAGTTTTTGGGGCGAAGTAGAGACTATGATAATTATATACAGATCTTTTCTGGTAAAGAAGGGGAGGGGGTT
CTTGAATTATTTTATCGCGGATTGATGTTAATAACGACCAGCTATGAAACTATCATTTTTATAATTTTAACATGTTC
TTTTTTTATAAAGGCAAGGTTTCTCGCTAACTATTCGCGTAATTTTTCAGGCTTGACCTTATTCTTTATTTATTATG
CAAGCGTTGCACTTTGGGTTTTAGATTATACTCAATTCAGAAATGGTCTATGTATTTCCATTTTAATGTTTTCCGTA
TACTATTTATTTATAAATAAACCGACTTATTTTTATTTCTCGGTATTATGTGCAATTGCAACTCATTGGTCTGCTTT
GCCTTTTTTGCTTTTATATCCTTTTGTCTATTCAACAAAAATAAGACGCCTTGGTTATTTTTGTTTCAGTATTCTTG
TTTTGATTGCGATCTCAGGAGAAGGAAAAGAGATCATATCTTTTATAAGAAATTTTGGAGTGGGACAAAAAATAGGA
AATGAAGCTGGTGTAAATTTAATAAATTCATTATCCCTTACCGCTATTTCCTGGTTTATTATTAGTTACATATCAAG
CATTGGAAATGAAAGGAGAAATTTAAGGCTTTTCTTTTGTTATGGTGTCATGCAATACGTGACTTTTAGCCTTTTCT
CTCTACCTGTTATGGCTTTCCGTATTTTGGAAATGTATTTTTTCCTTATGCTAACCATTGGGGTGTTTATTAAGCAA
AAAAAGAATTATTATTTTATTTTTTGCAAAGTGTTAATTTTATTGTATCTAACATACTATTATCATATGGTCTTTGG
AGTGATTAATGTGTAAGGCTAAGGTGTTGGCTATAATTGTTACTTACAACCCGGAAATTATTCGATTGACGGAATGT
ATTAACTCTTTAGCCCCACAAGTTGAGAGAATAATTCTTGTAGATAATGGCTCAAATAATAGTGATTTGATAAAAAA
TATCAGTATTAATAACCTTGAAATTATTTTACTTTCGGAAAACAAAGGCATTGCATTTGCTCAGAACCATGGTGTTA
AGAAGGGCCTGGAAGCAAAAGAGTTTGACTATTTATTTTTCTCAGATCAGGATACTTGCTTTCCTAGCGATGTTATT
GAAAAACTTAAGAGTACATTTACGAAAAATAATAAAAAAGGTAAAAATGTTGCTTGTGCTTCTCCTTTTTTTAAAGA
CCATCGTTCAAATTATATGCATCCGTCAGTCAGCCTAAATATTTTTACGAGTACAAAAGTTATATGTAGTGAAGTAG
ACGATGATCTTTATCCCTCGCATGTTATTGCTTCTGGGATGTTAATGTCTCGTGAAGCATGGCGCGTCGTCGGACCA
TTTTGTGAAAAACTCTTTATAGACTGGGTTGATACAGAATGGTGTTGGCGTGCATTAGCTAATAATATGATTATTGT
TCAGACACCATCAGTCATCATTTCTCATGAACTTGGGTATGGGCAGAAAATTTTTGCTGGTCGATCTGTTACAATAC
ATAATTCTTTCAGAAATTTTTATAAAATACGCAATGCAATATACTTAATGCTGCATTCAAATTATAGCTTCAAGTAT
CGTTATCATGCTTTTTTTCATGCGACAAAGAATGTTGTATTTGAAATTTTATATTCGAAAGAAAAATTAAATTCACT
GAAGGTTTGTTTTAAAGCTGTACGTGATGGTATGTTCAATAATTTTTAATACGAAAATAGTTAGGCTCAAGGTGTTT
AAATGGAAGAAAATAATATGAAGACGGTCGCTGTAGTTGGCACAGTGGGTGTTCCTGCTTGTTATGGTGGGTTCGAA
TCACTTGTTCAGAATCTAATTGATTATCAATCTGATGGTATACAATATCAGATATTTTGCTCTTCAAAAAAATATGA
TAAAAAATTTAAAAATTATAAAAATGCAGAATTAATCTATTTGCCGATAAATGCCAATGGCGTCTCTAGCATAATTT
ATGATATTATGTGTTTAATTATTTGTTTATTCAAAAGGCCAGATGTTGTTTTAATATTGGGGGTGTCTGGTTGTTTA
TTTCTACCAATTTATAAACTATTTTCAAAATCAAAGATTATTGTCAATATTGATGGGCTTGAATGGCGTAGAAATAA
ATGGGGAACGTTTGCTAAGAAATTTCTTAAAATATCTGAGGCGATATCTATTAGAATAGCTGATATTATCATTTCAG
ATAATCAAGCAATAGCTGATTATGTGGAAAATAAGTACAAGAAAAAAAGTGTAGTTATAGCTTATGGCGGAGATCAT
GCCACTAATCTTAGTACACCGATAGACAATGATCAAAAAAAAGAAGGTTATTATTTGGGGCTTTGTAGGATAGAGCC
TGAGAATAATATAGAAATGATTCTGAATGCCTTCATTAATACAGATAAAAAAATTAAATTTATGGGTAATTGGGATA
ACAGCGAGTATGGACGCCAGCTAAAAAAATATTATTCAAACTATCCAAATATCACCCTACTAGAACCTAACTATAAT
ATTGAAGAGCTTTATAAACTAAGAAAAAATTGTCTTGCATACATTCATGGACACTCGGCTGGTGGAACAAACCCTTC
TTTAGTTGAAGCGATGCATTTTAATATTCCTATTTTTGCTTTCGATTGTGACTTTAATCGTTACACAACTAACAATT
TAGCTCATTACTTTAATGATTCTGAACAACTTAGCTTATTAGCAGAAAGTTTGTCTTTTGGAAATCTTAAATGTCGA
GTATTAGATTTAAAAAATTATGCTGAAGATATGTATAACTGGAGGCATATAGCTGCTATGTATGAATCTATTTATTA
AACGCATTAACAATAATATAATTGACCTTATATAGCAGGGAAAGATCACGTAACGCTGCGGCGCGCCGATCCCCATA
TGAATATCCTCCTTAGTTCCTATTCCGAAGTTCCTATTCTTTCTAGAGAATAGGAACTTCGGAATAGGAACTAAGGA
GGATATTCATATGGATAAAGCCGTAAGCATATAAGCATGGATAAGCTATTTATACTTTAATAAGTACTTTGTATACT
TATTTGCGAACATTCCAGGCCGCGAGCATTCAGCGCGGTGATCACACCTGACAGGAGTATGTAATGTCCAAGCAACA
GATCGGCGTAGTCGGTATGGCAGTGATGGGACGCAACCTTGCGCTCAACATCGAAAGCCGTGGTTATACCGTCTCTA
TTTTCAACCGTTCCCGTGAGAAGACGGAAGAAGTGATTGCCGAAAATCCAGGCAAGAAACTGGTTCCTTACTATACG
GTGAAAGAGTTTGTCGAATCTCTGGAAACGCCTCGTCGCATCCTGTTAATGGTGAAAGCAGGTGCAGGCACGGATGC
TGCTATTGATTCCCTCAAACCATATCTCGATAAAGGAGACATCATCATTGATGGTGGTAACACCTTCTTCCAGGACA
CTATTCGTCGTAATCGTGAGCTTTCAGCAGAGGGCTTTAACTTCATCGGTACCGGTGTTTCTGGCGGTGAAGAGGGG
GCGCTGAAAGGTCCTTCTATTATGCCTGGTGGCCAGAAAGAAGCCTATGAATTGGTAGCACCGATCCTGACCAAAAT
CGCCGCCGTAGCTGAAGACGGTGAACCATGCGTTACCTATATTGGTGCCGATGGCGCAGGTCACTATGTGAAGATGG
TTCACAACGGTATTGAATACGGCGATATGCAGCTGATTGCTGAAGCCTATTCTCTGCTTAAAGGTGGCCTGAACCTC
ACCAACGAAGAACTGGCGCAGACCTTTACCGAGTGGAATAACGGTGAACTGAGCAGTTACCTGATCGACATCACCAA
AGATATCTTCACCAAAAAAGATGAAGACGGTAACTACCTGGTTGATGTGATCCTGGATGAAGCGGCTAACAAAGGTA
CCGGTAAATGGACCAGCCAGAGCGCGCTGGATCTCGGCGAACCGCTGTCGCTGATTACCGAGTCTGTGTTTGCACGT
TATATCTCTTCTCTGAAAGATCAGCGTGTTGCCGCATCTAAAGTTCTCTCTGGTCCGCAAGCACAGCCAGCAGGCGA
CAAGGCTGAGTTCATCGAAAAAGTTCGTCGTGCGCTGTATCTGGGCAAAATCGTTTCTTACGCCCAGGGCTTCTCTC
AGCTGCGTGCTGCGTCTGAAGAGTACAACTGGGATCTGAACTACGGCGAAATCGCGAAGATTTTCCGTGCTGGCTGC
ATCATCCGTGCGCAGTTCCTGCAGAAAATCACCGATGCTTATGCCGAAAATCCACAGATCGCTAACCTGTTGCTGGC
TCCGTACTTCAAGCAAATTGCCGATGACTACCAGCAGGCGCTGCGTGATGTCGTTGCTTATGCAGTACAGAACGGTA
TTCCGGTTCCGACCTTCTCCGCAGCGGTTGCCTATTACGACAGCTACCGTGCTGCTGTTCTGCCTGCGAACCTGATC
CAGGCACAGCGTGACTATTTTGGTGCGCATACTTATAAGCGTATCGATAAAGAAGGTGTGTTCCATACCGAATGGCT
GGATTAA
(example O6A rfb locus nucleotide sequence-O6A-EPA production strain
stGVXN4112 and stLMTB10923)
SEQ ID NO: 13
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATTTGCCCGCCGGGCGTGACAATTATGAACGTGCG
TCAGGGCGAACCTTTAGGTTTGGGCCACTCCATTTTATGTGCACGACCTGCCATTGGTGACAATCCATTTGTCGTGG
TGCTGCCAGACGTTGTGATCGACGACGCCAGCGCCGACCCGCTGCGCTACAACCTTGCTGCCATGATTGCGCGCTTC
AACGAAACGGGCCGCAGCCAGGTGCTGGCAAAACGTATGCCGGGTGACCTCTCTGAATACTCTGTCATCCAGACCAA
AGAGCCGCTGGACCGCGAAGGTAAAGTCAGCCGCATTGTTGAATTCATCGAAAAACCGGATCAGCCGCAGACGCTGG
ACTCAGACATCATGGCCGTTGGTCGCTATGTGCTTTCTGCCGATATTTGGCCGGAACTTGAACGCACTCAGCCTGGT
GCATGGGGGCGTATTCAGCTGACTGATGCCATTGCCGAACTGGCGAAAAAACAGTCCGTTGATGCCATGCTGATGAC
CGGCGACAGCTACGACTGCGGTAAAAAAATGGGTTATATGCAAGCGTTCGTGAAGTATGGACTACGCAACCTCAAAG
AAGGGGCGAAGTTCCGTAAAGGGATTGAGAAGCTGTTAAGCGAATAATGAAAATCTGACCGGATGTAACGGTTGATA
AGAAAATTATAACGGCAGTGAAGATTAGCGGCGAAAGTAATTTGTTGCGAATTTTCCTGCCGTTGTTTTATATAAAC
AATCAGAATAACAACGACTTAGCAATAGGATTTTCGTCAAAGTTTTCCAGGATTTTCCTTGTTTCCAGAGCGGATTG
GTAAGACAATTAGCATTTGAATTTTACGGGTTTAGCGCGAGTGGGTAACGCTCGTCACATCGTAGACATGCATGCAG
TGCTCTGGTAGCTGTAAAGCCAGGGGCGGTAGCGTGCTGAAATTATAAAGTCATTCTTATAGAACATCGCATTTCAA
TAATATAATTACACCTAAATGAATAGGATACAACGTGTGCACAATTATTTAAGGCTTAAAGATAAAATAAAAAACGT
ATTTTTAGGGTTGTATATATTGCAGTTATTTAATTATATCGCGCCATTGGTAATTATCCCTATCCTGATAAAATATA
TTGGGTTGGGGGAATATGGGGAATTGGTCTATATTACATCTATTTATCAAATAGTGGCTTTGATTATTGATTTTGGC
TTTACTTACACAGGACCTGTGGTTGCTGCGAGACATAGATGTGAGACCCAAAATTTACAGCGCTATTACTCAATAGT
TGTTCTTTTAAAATCATTGCTTTTTATAATTGCATTAACATGTGTATTTTTATTGTGCAGATTAAATATAGTCCACT
TGTCATTTTTTGGGTTTTTGTCAATTTTTCTATGCACTATTGGTAATATATTATCGCCCAATTGGTTTTTGCAGGGG
ATTGGTGATTTTAAAAAACTTTCATACTCACAAGTAATAGTGAGAATAACATTGTTTATCATACTTCTTGTTTATGT
CTGTAGTGGCGGAGATAATGTTTTTATCCTAAGTTTTTTGCAAAATGCAACATTACTCATATGCTGTATATACTTAT
GGCCAAATATTCATATTAGCCATGTTGTTCATCTTAAACCTAATGAATGCATTGTGGAATTTAAGAAGGCAGGAAAT
GTTTTTATTGGCGTAATAGGTACGATTGGTTACAATGGTCTAATTCCTGTGTTAATTGGAAACCTTTGCGGTAATAC
GAGTCTTGGTGTTTTTTCAATCGTTCAAAAAATGACAACAGCATGTCAAAGTCTAATTAATCCAATATCACAGTATA
TGTTATCTCAAGTTTCAGAAATTAAACCTCAAGATAAACTGTTTTATTATAGAATTAAAAAAAGTTTTTTTGTGCAT
TTAACAATTAGCATAATTGCATGTTTATGTTATATGGGGTTAGGGCAATATGTGGCGACTTTTATAGGTAAAGTTGA
CGTTTCATTTGTTATTATTTTATTTGCGTCAATAATTACCATTTTTTCATCTTTAAATAATGTCCTTGGTATACAGT
TTCTTATACCGACAGATAATGTAAAAATACTACGAAGTATAAATGTTATGGCGGGAATTATTGTTGTTAGTTTGTCC
TGGCTGTTAATATCACGCTTTGACATTCTGGGGGGGGTTTTATTAAACCTAATTGGTGAGTTTCTTGTATTCAGTAT
GCTAGCTTTTATTGCCCATCGAAAGTGGGGAGCGAGAGTATAATGAAAGTGAAGGCGGTTCCTGCTATTACATTCTA
TTTAAGTTTAATGCTGACAATTTTAGTGTTACTGTTTGGTAATGAACCAAATAAATCACAATATATCCTTGTTATAG
CAACGATAACAGTTTTTTATATCGCATATATCACTAATAAAATAACTTCTCCGGCCAGCCTTCTCGTTATATCATCT
TTTGTGTTTTTAGGTTGTCGCCCTTTATTATCTTTGTTTGCAAACTATGATTATAGGATTGCCGATTGGTTTATTGA
AGGATATATGGATGACGATGTGATTTTGGCTAACTATGCTATAACACTAATGTATTATGGTTATACATTGGGACTAA
TTCTATGCAAAAATACTGAAAAATTTTATCCGCATGGTCCTTATCCTGAAAAACAATTGCTAAAAATAAAGTTTCTT
TTGACTTTATTTTTTCTGGGTTCGATAGGTATGGTTGTAAAAGGGATATTCTTTTTTAACTTTATAGAATCTAATAG
TTATGTTGATATTTATCAATCAAATATAACAACGCCAATAGGTTATGATTTTCTATCTTATTTATTTTATTGTTCTT
TTTTCCTTATATGTGCGTTTCATATACAGTTCAGAACAAATAAAAAATTTCTTTTTATTGCGATATGCATTGCTGCA
TTTAGCACCTTGAAGGGTAGTCGTAGTGAAGCTATAACGTTTCTTTTAACGGTTACATGTATATATTTTAATGAAGT
AAAGACAAGAAACTTACGTCTGCTGATTACAATGATTTTTGTTTTTAGCGTCATTTTTGTGATTAGTGAATTTATCT
CAATGTGGCGCACTGGAGGGAGTTTTTTTCAATTAATGCAGGGTAATAATCCTGTTATAAACTTTGTATACGGCATG
GGAGTATCATATCTTTCCATTTATCAATCAGTAAAACTACAACTATTGTCAGGGGGATATAATGTTACCTATCTATT
CAGCCAGTTAATAATAACTTGCTCGTCAATATTTAATGTCAAATTGAGCTTGCCGGAAATAAGCTATAGCCATTTGG
CCTCATACACAGCAAACCCAGAACTATATAATCTTGGGTTCGGACTTGGGGGGAGTTATTTAGCAGAATCGTTTTTA
GCATTTGGTCTGATTGGATGTTTCATTATACCCTTTTTACTTTTACTTAATTTAAATGTATTGGAAAAATATACAAA
AAACAAACCAATTATATATTTTGTTTATTATAGTGTGTTGCCACCTATATTATTCACACCAAGAGAGACTTTGTTCT
ATTTCTTCCCCTATCTTGTCAAAAGTATATTTGTTGCTTTTTTAGTTACATTATACATCCAGTATAAAAAGGATTGA
CCAAAATGTCAGAAAAAAATGTCAGCATAATAATCCCAAGTTATAACAGGGCTCATATTCTTAAGGAGGTCATACCA
AGTTATTTTCAGGATGAGACTTTAGAGGTTATAGTTATCAATGATGGATCAACAGATAATACAAATAGTGTATTAGC
TGAACTGAAGGAAAAATATTCTCAGTTAGTTATTTTAGAAAATGAAACGAATAAAAAACAGATGTATTCTAAAAACC
GAGGGATTGAAATAGCCAAAGGGAAATATATTTTTTTTGGTGATGATGACTCTTACCTCTTACCCGGTGTTATATCT
CGGTTATTGGCTACAAAATATGAGACAGGCGCTGATGTAATCGGCGCAAGAATACTTTATATGAATAATAACGAGAA
AACAATTGAAGATTGCATAAATCGACATAAAAAAGAGGGGCGTTTTGTTAGTGATCTAAATAGATTGGATTTTAGTT
ATACATGTGATTTGGACCATCCGATTGAATGTTTTTATGCACAGCCTTTTGTTCTAGCTGAAAGGGAACTAATATCG
AAATATCGATTTGATATATCTTATACGGGAAACTGCTATCGTGAGGAAACTGATTTCATGCTATCTCTATTTATTAA
AAATAAAAAATTTATATATGATTCAAAGGCTTTGTTAATAAATTTACCTCCAAGAAAAGCGACGGGAGGGGCAAGAA
CAGCTAATCGATTAAAATATCATTACGAAAGTTGCATAAATAATTATAGATTTTTAAAAAAATATAATGATAATTTG
AATCTTCTTTCAGGACAAAAGCATGCTATATTTTACCGACAGTGTCAATTCGTTCTGCTAAAAATGAAGTCGTTTAT
CGGGAAGTTTTTAAAATGATTATATATATCGCCGCGTATAATGGTTCAGGAGGGCAAGGTGGGGTGGAAAGGGTTGT
TGCCCAACAATGTAACATTCTTAAAAATTTGGGGGTTAAAGTCATTATACTTGATAAAACATACTTCAAAATTTCTA
ACAAAATTCGTAACAAAAAAATACAAGTAGCACTTTATCCAATATTAGTTTCTCTTTATTTAACCTTACAAAAATTA
CGTGGCGTGACGTTTAAAGTTATTGCACATGGCTATTGTTCTCCTTTTTATAGGAATGACATCTTAATAGCTCATGG
CAATATGAAATGTTATTTTCAAACAGTCATGAATAAAAAACCTAATCGGTTGTCTGGCAGTGGTCTTTTATCTTTCT
ATGAGCGTTGGGCTGGAGCATTTTCAAAAAATATCTGGGCTGTTTCAAATAAGGTTAAAAGTGAATGGAATGAGCTT
TACAATATTAATTCACATAAAATCAAAGTTGTTCGAAATTTTATAAATCTTGCACAATTTGATTACACTGATGTTAA
TGAAGCAGAATATGTGACATTTGTCGGGCGATTGGAAAAAGGAAAAGGAATAGATGATCTGTATTACATATGTAAAA
ATCTGCCAGATACTTCCTTCCATTTAGTTTCAAGTATTCCCGCCCCACAAAATTTTGCTTCGCTAAATAATGTTCTG
ACCAGCATTGCTGTCCCCTATGCGAAAATGCCAGAAATATTTAAGAAATCCAGAGTACTTATTTTACCGTCCTATTA
TGAAGGATATGAGCTGGTTACTATTGAAGCGCTATGCTGTGGTTGCCCTGTGATAGGCTATAATGTTGGTGCAATTA
GAGAGTTGTATGCAGAAAGTTTTCCTGGCGTATTTATTGCCAATAATAAAGAAGATTTAGCACAAGTAGCCTACAAA
TTAATTAGTCTTGATAATGAAAAATATTATCATTTGAGACAAACTATTTATAGCAAGCGTGAGCTTTTTTCTGAAGA
GAGATATGCGGAAATTTTAACGGCGGCATTTAATGAAAAAAAATAAGAAACTCTGTCTCATTTCAATTAACTCATAT
AATGAACTTACCGGAGGAGGAGTATATTTACGTACGCTTGTTAGTTTTCTACAAAAACAGAATGTTAATTTAACACT
TATTGATAAAAAATCCTCAGGTAAACTATTCGAAGACAATACTTTTCAACATATATCATTTATTAAAGGTAAACGTC
AGGATATAATATCCAGGCTTTTTTTTATACCATCATTTTATGTCCCTTATATTTTCTCAATAATTAAAATTTTACGG
AAGCAAGATATTCTTGCTTTTCACAACTCTCGGCTTGGATTGTTATGTCTGCTTTTTAGAATACTCATGCCCCACAA
AAAGATCATATTGTTTACGGATAACTTCGAATATGACTTAATAAGACAAAAAGATAAAAACATAACTACTTTTATTG
AAAAATTAATTGTTTATCTCAATGAATTTATCGGGCTTAAGAATTCAGATTTAGTTAGCTATATTACCCGGCAAGAT
AAAAATGCAATGGATAAATTTTATGGGATTAAAAAAAGCAGAAATTTAATTCTCCCTGTGATATTTAGTAGAGAAAA
ACCAACTGATGTATTGTCAGCTCACTTTATTAATGAGTATAATCGATTGAATAATGATAATAGGAAAAAAGTAGTAT
TTACTGCATCTTTTGATTTTTTTCCAAATATAGATGCTGCCAACTATGTTTTAAATGCAGCAAAGTCTAATAATGAT
TATTGCTATATTTTGGCAGGTAGGAAAAGTACTACTTTGAATCTTCCTGATTTGGATAATTTATTTTTTTTCGATAA
TCTATCTAATAGTGAAATGTCATATTTATTATCTGCTTGTGATGTTTTTTATTCTCCTATAGTTTTAGGAAGTGGAA
TGAAAACAAAAATTGCAGAAGCACTATCATATGGATTATATATTTATGCGACAGAGCATTCCTTAATCGGCTATGAT
GAAATTATACACAATAAGGAGTGTGTTAAAAAAATCTCACATTTGGATGAGGAATTTCCTAAAGATTTCAAGATGAA
AAGTATCAATAAACAGCTAATAATGTCTTATCAGCAAAAATATTATTCACATTATCGGTTTAATGGCCATGAACTTG
ATATAATAAATTTTGACGATTAGTTAGTGGAGATATAATATGAACATATTAGTAACTGGTGGTGCTGGATATATCGG
ATCTCATACGGCTATTGAATTACTGAATGCAGGTCATGAGATTATCGTTCTGGACAATTTCAGTAATGCTTCATACA
AGTGTATCGAAAAAATAAAAGAAATTACTCGACGTGATTTTATAACAATTACTGGAGATGCTGGGTGTAGGAAGACA
CTCTCCGCTATTTTCGAGAAACACGCCATAGATATAGTTATTCATTTTGCTGGCTTTAAATCTGTTTCAGAGTCTAA
AAGTGAACCCTTAAAGTATTACCAGAATAATGTTGGAGTGACCATTACTTTATTACAGGTAATGGAAGAGTACAGAA
TTAAAAAATTTATCTTTAGTTCATCTGCGACAGTCTATGGTGAACCAGAGATAATTCCAATTCCAGAAACAGCTAAA
ATTGGAGGAACTACGAATCCATATGGCACATCGAAGTATTTTGTTGAAAAAATTCTAGAGGATGTTAGTTCCACGGG
AAAACTGGATATAATTTGCTTGAGATATTTTAATCCTGTCGGTGCTCATTCTAGTGGTAAAATAGGTGAGGCTCCAT
CTGGTATCCCTAATAATCTTGTTCCTTATTTATTGGATGTTGCGAGTGGTAAACGTGATAAATTATTTATTTATGGC
AATGATTACCCTACTAATGATGGAACAGGTGTAAGGGATTTTATTCATGTTGTTGACTTAGCGAAAGGTCATTTGGC
TGCAATGAATTATTTAAGTATCAATTCGGGATATAATATCTTTAATCTTGGTACAGGAAAAGGTTATTCGGTACTTG
AATTAATCACTACATTTGAAAAATTAACAAACATTAAGGTCAATAAATCTTTTATAGAGAGAAGGGCAGGGGATGTT
GCGTCTTGTTGGGCTGATGCAGATAAAGCTAATTCTTTATTGGACTGGCAAGCCGAACAAACTCTAGAACAGATGTT
ATTGGACTCGTGGCGTTGGAAAAAAAATTATCCAGACGGATTCTGAATATAAAAGGTTTCAGTTTTATGAATCAATC
AGAGCAGAGAAAAAAAATACTGGTTCTTACACCTCGCTTTCCCTACCCTGTCATTGGAGGGGATAGATTAAGAGTCT
ATATGTTATGTAAAGAACTTTCCAAAAAATATGATCTTATTCTTCTGAGCTTATGTGATCAACCACTAGAACTTGAA
ATAAATATAAATGACTCGGTCTTCAAAGAAATTCATCGTGTCTATCTACCAAAATATAAATCATATTATAATGTATT
AAAAGCTTTGGTTACGCAAAAACCGTTGCAAATTGCTTATTATCAATCGGACACATTTAAGAATAAATACAATAAAT
TAATTAAACAATGCGATGCAGTATTTTGTCATCTGATAAGAGTTGCTGATTATGTTAAGGATACAGACAAGTTCAAA
ATTCTTGATATGACAGATGCAATATCTTTGAATTACAGTCGCGTTAAAAAATTAGCAAGTAAAAAAAGTTTGCGTGC
AATTATTTATTCTCTGGAACAAAAAAGATTAGAATCATATGAACGTTCTGTGGCGAATCTTTTTGATTTGACCACTT
TTATTTCATCCGTAGACCGTGACTATCTCTACCCTAATCTGGGCAGTAATATCCATATAGTCAATAATGGGGTTGAT
ACATCAGCCTTGAGATATATAAAAAGAGAAATAAAAATCGATAAGCCTGTGGAACTTATATTTATCGGAAATATGTA
TTCTTTACAAAATATGGATGCTGCAAAACATTTTGCTAAGAATATTTTACCTTGCTTGTATGATGAGTTTAATATTA
TTTTTAAAGTGATTGGTAAGATCTCAGAAACTAATAAAAATATATTAAATTCATTTAAAAATACAATTGCTTTAGGT
ACTGTTGATGATATCAATTCTTCCGCTTCTACAGGGCATATAGGTATATGTCCTGTTCGTCTTGGAGCAGGCGTACA
AAATAAAATTCTTGAATACATGGCTTTAGGTTTACCATGTATTACATCTAGCATTGGTTATGAAGGTATTAATGCAA
AATCAGGTAGCGAAATTTTTGTTGCAGATACAGTAGAGCAATATAAAAACGTACTAAGAGAAATAATTTACGATTAT
AATCGTTATACTGAAGTGGCTGAAAATGCCCGTAGTTTTGTAGAAAATAATTTTTCTTGGGAATCAAAAGTTGCCAA
TTTAATGAATACATTAGATGAGAAATTATATGAACAATAATAAAATTATTACACCTATCATTATGGCTGGTGGTTCA
GGCAGTCGGTTGTGGCCACTATCAAGAATTCTCTATCCGAAACAATTTCTTAGCCTAATCGGTAGTCATACCATGCT
TCAAACAACGGCTAATCGTCTGGATGGTTTGGATTGTACCAACCCTTATGTCATTTGTAATGAACAATACCGCTTTA
TAGTTGCTGAACAGCTTAGAAAAATCGATAGATTGACTTCAAAGAATATCATCCTTGAGCCTGTTGGGCGTAACACT
GCCCCTGCAATTGCATTAGCGGCGTTGCTGATGTCTAAGTCTGATAAAAGTGCAGATGATCTTATGCTCGTACTGGC
TGCAGATCACGTTATACACGATGAAGAAAAATTTTGTAACGCTGTTAGATCGGCAATTCCATACGCTGCTGATGGGA
AATTGGTAACATTTGGTATAATTCCAGACAAAGCAGAAACTGGTTATGGTTATATACATCGAGGACAATATATTAAT
CAGGAAGATTCGGATGCATTTATAGTGTCATCATTTGTTGAAAAGCCAAATCATGAGACAGCCACTAAATATCTTGC
TTCCGGTGAGTATTATTGGAATAGCGGTATGTTTTTGTTTAGTGCAAATCGTTATATAGAGGAACTTAAACAATTTC
GGCCTGATATTTTATCCGCTTGTGAAAAAGCAATTGCTTCAGCGAACTTTGACCTTGATTTTGTGCGTTTAGATGAA
AGTTCTTTCTCTAAGTGCCCTGAAGAATCAATTGATTACGCTGTAATGGAAAAAACAAAAGACGCAATTGTTATTCC
AATGGATGCTGGCTGGAGTGATGTCGGTTCATGGTCTTCTCTTTGGGAAATTAATGATAAAGACTCAGACGGCAACG
TAATAGTTGGGGATATTTTCTCTCATGAAACAAAGAATTCTTTCATATATGCCGAATCGGGAATTGTTGCTACAGTT
GGAGTGGAAAATTTAGTTGTTGTCCAAACAAAGGATGCTGTTCTTGTCTCAGAGAGAAATAAAGTTCAGGATGTAAA
GAAAATAGTAGAACAAATTAAAAATTCAGGTCGTAGCGAGCATTATGTTCATCGCGAAGTATATCGTCCTTGGGGTA
AATATGATTCCATTGACACAGGGGAGCGTTATCAGGTCAAACGTATAACAGTAAATCCTGGTGAAGGACTTTCTTTA
CAAATGCACCATCATAGGGCAGAACATTGGATCATAGTTTCTGGAACTGCAAGGGTGACTATAGGTTCTGAAACTAA
GATTCTTAGCGAAAATGAATCTGTTTACATACCTCTTGGTGTAATACACTGCTTGGAAAATCCAGGGAAAATTCCTC
TTGATTTAATTGAAGTTCGTTCTGGATCTTATTTAGAAGAAGACGATGTTATCCGTTTTCAGGACCGATATGGTCGT
AGCTAAATTTTTGATAATGTAACGTTAGTAGAAGAGCGCTAATATTTTTAGTTAATCTGTAATAAGTATTATTTGTT
TAAGGTATATCATGTCGAGTTTACCCTGCTTTAAAGCCTATGATATTCGCGGGAAATTAGGCGAAGAACTGAATGAA
GATATTGCCTGGCGCATTGGTCGCGCTTATGGCGAATTTCTCAAACCGAAAACCATTGTGTTAGGCGGTGACGTCCG
ACTCACCAGCGAAACCTTAAAACTGGCGCTGGCGAAGGGGTTACAGGATGCGGGCGTCGATGTGCTGGATATTGGCA
TGTCCGGCACCGAAGAGATCTATTTCGCCACGTTCCATCTCGGCGTGGATGGCGGCATCGAAGTTACCGCCAGCCAT
AACCCGATGGATTACAACGGCATGAAACTGGTGCGCGAAGGGGCTCGCCCGATCAGCGGTGATACCGGACTGCGCGA
CATCCAGCGTCTGGCAGAAGCCAACGACTTTCCTCCCGTTGATGAAACCAAACGCGGTCGCTATCAGCAAATCAATC
TGCGTGACGCTTACGTTGATCACCTGTTCGGTTATATCAACGTCAAAAACCTCACGCCGCTCAAGCTGGTGATTAAC
TCCGGGAACGGCGCGGCGGGTCCGGTGGTGGACGCCATTGAAGCCCGCTTTAAAGCCCTCGGCGCACCCGTGGAATT
AATCAAAGTGCACAACACGCCGGACGGCAATTTCCCCAACGGTATTCCTAACCCGCTACTGCCGGAATGTCGCGACG
ACACCCGCAATGCGGTCATCAAACACGGCGCGGATATGGGCATTGCCTTTGATGGCGATTTTGACCGCTGTTTCCTG
TTTGACGAAAAAGGGCAGTTTATTGAGGGCTACTACATTGTCGGCCTGCTGGCAGAAGCGTTCCTCGAAAAAAATCC
CGGCGCGAAGATCATCCACGATCCACGTCTCTCCTGGAACACCGTTGATGTGGTGACTGCCGCAGGCGGCACCCCGG
TAATGTCGAAAACCGGACACGCCTTTATTAAAGAACGTATGCGCAAGGAAGACGCTATCTACGGTGGCGAAATGAGC
GCCCACCATTACTTCCGTGATTTCGCTTACTGCGACAGCGGCATGATCCCGTGGCTGCTGGTCGCCGAACTGGTGTG
CCTGAAAGGAAAAACGCTGGGCGAACTGGTGCGCGACCGGATGGCAGCGTTTCCGGCAAGCGGTGAGATCAACAGCA
AACTGGCACACCCCGTTGAGGCGATTAACCGCGTGGAACAGCACTTTAGCCGCGAGGCGCTGGCGGTGGATCGCACC
GATGGCATCAGCATGACCTTTGCCGACTGGCGCTTTAACCTGCGCTCCTCTAACACCGAACCGGTGGTGCGGTTGAA
TGTGGAATCGCGCGGCGATGTACCGCTGATGGAAGAAAAGACAAAACTTATCCTTGAGTTACTGAACAAGTAATTCA
GTAATTTCATATAAATGGGTTTTAAAAAACGGAAAAGATGAGATATCCGGTGTGGTATATCCAAGGTAATGCTATTC
AGTATCTCTATGAGTGAGTTAACATCTATACCACATTTAAGCCGCACACTTCGGGATCCCCATATGAATATCCTCCT
TAGTTCCTATTCCGAAGTTCCTATTCTTTCTAGAGAATAGGAACTTCGGAATAGGAACTAAGGAGGATATTCATATG
GATAAAGCCGTAAGCATATAAGCATGGATAAGCTATTTATACTTTAATAAGTACTTTGTATACTTATTTGCGAACAT
TCCAGGCCGCGAGCATTCAGCGCGGTGATCACACCTGACAGGAGTATGTAATGTCCAAGCAACAGATCGGCGTAGTC
GGTATGGCAGTGATGGGACGCAACCTTGCGCTCAACATCGAAAGCCGTGGTTATACCGTCTCTATTTTCAACCGTTC
CCGTGAGAAGACGGAAGAAGTGATTGCCGAAAATCCAGGCAAGAAACTGGTTCCTTACTATACGGTGAAAGAGTTTG
TCGAATCTCTGGAAACGCCTCGTCGCATCCTGTTAATGGTGAAAGCAGGTGCAGGCACGGATGCTGCTATTGATTCC
CTCAAACCATATCTCGATAAAGGAGACATCATCATTGATGGTGGTAACACCTTCTTCCAGGACACTATTCGTCGTAA
TCGTGAGCTTTCAGCAGAGGGCTTTAACTTCATCGGTACCGGTGTTTCTGGCGGTGAAGAGGGGGCGCTGAAAGGTC
CTTCTATTATGCCTGGTGGCCAGAAAGAAGCCTATGAATTGGTAGCACCGATCCTGACCAAAATCGCCGCCGTAGCT
GAAGACGGTGAACCATGCGTTACCTATATTGGTGCCGATGGCGCAGGTCACTATGTGAAGATGGTTCACAACGGTAT
TGAATACGGCGATATGCAGCTGATTGCTGAAGCCTATTCTCTGCTTAAAGGTGGCCTGAACCTCACCAACGAAGAAC
TGGCGCAGACCTTTACCGAGTGGAATAACGGTGAACTGAGCAGTTACCTGATCGACATCACCAAAGATATCTTCACC
AAAAAAGATGAAGACGGTAACTACCTGGTTGATGTGATCCTGGATGAAGCGGCTAACAAAGGTACCGGTAAATGGAC
CAGCCAGAGCGCGCTGGATCTCGGCGAACCGCTGTCGCTGATTACCGAGTCTGTGTTTGCACGTTATATCTCTTCTC
TGAAAGATCAGCGTGTTGCCGCATCTAAAGTTCTCTCTGGTCCGCAAGCACAGCCAGCAGGCGACAAGGCTGAGTTC
ATCGAAAAAGTTCGTCGTGCGCTGTATCTGGGCAAAATCGTTTCTTACGCCCAGGGCTTCTCTCAGCTGCGTGCTGC
GTCTGAAGAGTACAACTGGGATCTGAACTACGGCGAAATCGCGAAGATTTTCCGTGCTGGCTGCATCATCCGTGCGC
AGTTCCTGCAGAAAATCACCGATGCTTATGCCGAAAATCCACAGATCGCTAACCTGTTGCTGGCTCCGTACTTCAAG
CAAATTGCCGATGACTACCAGCAGGCGCTGCGTGATGTCGTTGCTTATGCAGTACAGAACGGTATTCCGGTTCCGAC
CTTCTCCGCAGCGGTTGCCTATTACGACAGCTACCGTGCTGCTGTTCTGCCTGCGAACCTGATCCAGGCACAGCGTG
ACTATTTTGGTGCGCATACTTATAAGCGTATCGATAAAGAAGGTGTGTTCCATACCGAATGGCTGGATTAA
(example O8 rfb locus nucleotide sequence-O8-EPA production strain
stLMTB11734)
SEQ ID NO: 14
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGCTAGCGATCGCTTAAGATCTAGGATTTCATTATGTTACTTCC
TGTAATTATGGCTGGTGGTACCGGCAGTCGTCTCTGGCCGATGTCACGCGAGCTTTATCCGAAACAGTTCCTCCGCC
TGTTCGGGCAGAACTCCATGCTGCAGGAAACCATCACCCGACTCTCGGGCCTTGAAATCCATGAACCGATGGTCATC
TGTAACGAAGAGCACCGCTTCCTGGTGGCTGAACAGCTACGCCAGCTCAATAAGCTGTCGAATAATATTATTCTTGA
GCCGGTCGGGCGCAACACCGCCCCGGCCATCGCCCTGGCAGCCCTTCAGGCCACCCGCGACGGCGACGACCCGCTGA
TGCTGGTTCTCGCCGCTGACCATATCATCAATAACCAGTCGGCCTTCCACGACGCCATCCGGGTCGCCGAGCAGTAT
GCTGATGAAGGTCATCTGGTCACCTTCGGTATCGTGCCGAATGCCCCGGAAACTGGCTACGGTTACATTCAGCGCGG
CGTGGCGCTCACCGATAGTGCCCATTCCGCGTACCAGGTGGCCCGCTTTGTGGAGAAGCCGGATCGCGAGCGCGCCG
AGGCTTACCTCGCCTCCGGGGAGTACTACTGGAACAGCGGCATGTTTATGTTCCGCGCCAAGAAATACCTCATCGAG
CTGGCCAAATACCGTCCGGATATCCTGGAAGCCTGCCAGGCTGCGGTGAATGCCGCCGATAATGGCAGCGATTTCAT
CAATATCCCGCATGATATTTTCTGCGAGTGCCCGGATGAGTCCGTGGACTATGCCGTTATGGAGAAAACCGCCGATG
CGGTGGTGGTCGGTCTCGATGCTGACTGGAGCGACGTCGGCTCCTGGTCCGCACTATGGGAGGTCAGCCCGAAAGAC
GAGCAGGGCAATGTCCTCAGCGGTGACGCGTGGGTACACAACAGCGAAAACTGCTACATCAACAGCGACGAGAAGCT
AGTGGCGGCCATTGGCGTAGAGAATCTGGTGATTGTCAGCACTAAGGACGCCGTGCTGGTGATGAATCGCGAGCGTT
CCCAGGACGTGAAGAAGGCGGTCGAGTTCCTCAAGCAGAACCAGCGCAGCGAGTACAAGCGCCACCGTGAGATTTAC
CGCCCCTGGGGCCGTTGCGACGTAGTGGTCCAGACCCCGCGCTTCAACGTCAACCGCATCACGGTGAAACCAGGCGG
TGCCTTCTCGATGCAGATGCACCACCATCGCGCCGAGCATTGGGTTATTCTCGCCGGCACCGGTCAGGTGACTGTCA
ACGGTAAGCAGTTCCTGTTGTCCGAGAACCAGTCCACCTTTATTCCGATTGGCGCCGAGCACTGCCTGGAAAACCCT
GGCTGTATTCCGCTGGAAGTGCTGGAGATCCAGTCGGGGGCGTACCTTGGCGAGGACGACATTATTCGTATTAAAGA
CCAGTATGGTCGTTGCTAATTATTTTCGGGACAAGACGCAGAATGACACAGTTAACTTGTTTTAAAGCTTATGACAT
CCGTGGTGAACTGGGTGAGGAACTGAACGAGGACATCGCCTACCGTATCGGTCGCGCCTACGGCGAATTTCTGAAAC
CCGGGAAGATAGTGGTGGGGGGCGATGTGCGCCTCACAAGCGAGTCGCTGAAGCTGGCGCTGGCCCGCGGGTTAATG
GACGCCGGTACCGACGTGCTGGACATCGGCCTGAGCGGTACCGAAGAGATTTACTTTGCCACCTTCCACCTTGGGGT
AGATGGTGGCATCGAGGTGACCGCGAGCCACAATCCTATGAACTACAACGGCATGAAGCTGGTGCGCGAGAATGCGA
AGCCCATCAGCGGCGACACCGGCCTGCGGGATATCCAGCGCCTGGCGGAGGAAAACCAGTTCCCGCCAGTGGACCCG
GCGCGTCGCGGGACCCTGAGCAAGATATCGGTACTGAAGGAGTATGTTGACCATCTGATGAGCTACGTGGACTTCTC
GAACTTCACCCGTCCACTGAAGTTGGTGGTGAACTCCGGAAACGGGGCTGCGGGGCACGTGATTGATGAGGTGGAGA
AACGCTTCGCGGCGGCTGGGGTGCCGGTAACCTTTATCAAGGTGCATCACCAGCCGGATGGCCATTTCCCTAACGGT
ATCCCGAATCCGCTGCTGCCGGAGTGCCGCCAGGATACCGCCGACGCGGTGCGCGAGCATCAGGCCGACATGGGGAT
TGCCTTTGACGGCGACTTCGATCGCTGCTTCCTGTTCGATGACGAAGCTTCGTTTATCGAGGGGTATTACATTGTCG
GCCTGCTGGCTGAGGCGTTCCTGCAGAAGCAGCCGGGAGCGAAAATCATTCACGACCCGCGCTTGACGTGGAACACG
GTAGACATCGTGACCCGCAACGGCGGCCAGCCGGTGATGTCGAAGACGGGGCATGCGTTCATCAAGGAGCGGATGCG
TCAGGAAGACGCTATCTACGGCGGGGAGATGAGTGCGCACCATTACTTCCGCGATTTCGCCTACTGCGATAGCGGGA
TGATCCCGTGGCTGCTGGTGGCGGAGCTGCTGTGTCTGAAGAACAGCTCGCTGAAATCGCTGGTGGCGGACCGCCAG
AAGGCGTTCCCTGCGTCGGGAGAGATCAACCGCAAGCTAAGTAATGCTGCTGAGGCGATCGCCCGCATCCGGGCGCA
GTATGAGCCGGCGGCTGCACACATCGACACAACGGACGGGATCAGTATTGAATACCCTGAATGGCGCTTTAACCTGC
GCACGTCTAACACCGAGCCGGTGGTGCGTCTGAACGTTGAGTCCAGAGCTGATGTGGCGCTTATGAATGAAAAAACG
ACCGAGCTGTTACACCTGTTAAGCGGGGAATAAGGTGAGAGATTTACTAACGACGATTTATCGTTATCGGGGATTTA
TCTGGAGCAGTGTTAAACGTGATTTTCAGGCACGCTATCAAACTAGTATGCTGGGCGCACTATGGCTCGTTTTACAA
CCGCTCTCTATGATTCTGGTCTATACCCTGGTTTTTTCCGAGGTGATGAAGGCAAGAATGCCCGATAATACCGGGTC
GTTTGCCTATAGTATTTATCTCTGTTCCGGGGTACTGACCTGGGGATTATTTACTGAGATGCTGGATAAAGGTCAGA
GCGTATTTATTAACAATGCTAATCTGATCAAGAAACTCAGTTTTCCGAAAATCTGTCTGCCGATCATCGTGACGTTA
TCGGCGGTGCTAAATTTCGCGATTATTTTCAGTCTGTTTCTAATTTTTATCATTGTCACCGGTAACTTCCCCGGCTG
GCTCTTTCTCTCGGTGATACCGGTCCTGCTTTTGCAGATCCTGTTTGCCGGTGGGCTGGGGATGATCCTTGGTGTCA
TGAACGTCTTTTTCAGGGATGTGGGGCAACTGGTTGGCGTTGCGCTGCAATTCTGGTTTTGGTTCACACCCATTGTT
TATGTACTGAATTCATTACCTGCATGGGCAAAAAATCTGATGATGTATAACCCGATGACTCGGATCATGCAATCTTA
TCAGTCCATCTTCGCCTATCATCTGGCCCCCAACTGGTATTCGCTATGGCCAGTATTGGCTCTCGCCATTATTTTCT
GCGTCATCGGTTTCAGGATGTTCCGCAAGCATGCGGCGGATATGGTGGATGAATTATAATGAGTTATATCAGAGTAA
ATAATGTCGGTAAGGCGTATCGCCAGTATCACTCAAAGACCGGGAGACTGATCGAATGGTTATCCCCTCTGAATACC
AAACGCCATAATTTGAAATGGATCCTCCGCGATATTAATTTCGAAGTCGCTCCGGGCGAGGCTGTCGGTATTATCGG
TATCAACGGTGCAGGCAAGAGTACCCTGCTTAAACTCATAACCGGGACGTCCAGGCCGACGACTGGAGAAATTGAAA
TCTCCGGACGTGTCGCTGCATTACTCGAATTGGGGATGGGGTTTCATTCTGATTTCACTGGTCGGCAGAATGTTTAT
ATGTCTGGGCAACTGTTGGGGTTATCGTCAGAGAAAATAACTGAACTGATGCCGCAAATTGAAGAGTTTGCTGAGAT
TGGGGACTATATCGATCAACCTGTGCGCGTCTACTCCAGTGGGATGCAAGTTCGATTAGCTTTTAGTGTAGCGACGG
CTATCCGTCCTGATGTGCTAATTATCGATGAGGCATTATCTGTTGGGGATGCATATTTCCAGCATAAAAGCTTTGAG
CGTATTCGAAAATTTCGTCAGGAAGGGACCACGCTGTTGCTGGTATCCCATGATAAACAAGCGATCCAAAGCATTTG
CGACCGGGCCATTTTATTGAATAAAGGCCAAATTGAAATGGAAGGTGAACCTGAAGCAGTGATGGATTATTACAATG
CTCTTCTGGCCGATAAACAAAATCAGTCCATTAAACAAGTTGAGCATAATGGTAAAACGCAAACTGTTTCAGGCACT
GGTGAGGTGACTATCTCTGAGGTTCATCTTCTCGATGAACAGGGCAATGTGACTGAATTTGTTTCGGTAGGGCATCG
TGTCAGCTTGCAGGTCAACGTTGAGGTCAAGGACGATATTCCTGAGCTTGTTGTCGGATATATGATTAAGGATCGAC
TTGGGCAGCCGATTTTCGGGACCAATACGTACCATCTCAATCAGACACTCACCTCCCTGAAAAAAGGAGAAAAGCGT
TCGTTCTTATTTTCTTTCGATGCGAGATTGGGGGTTGGCTCCTATTCTGTCGCTGTCGCGTTGCATACTTCCAGTAC
GCACCTCGGCAAAAACTATGAATGGCGCGATCTGGCCGTGGTATTCAACGTCGTTAACACGGAACAACAAGAGTTTG
TCGGCGTGTCCTGGTTGCCGCCTGAACTGGAGATTTCTTAATGGGTTCGTCGTTTTATCGTTCATTTGAAGAACGAC
ACAGAGGTTCGGTTGAAGAAATCAAGCGCCGCTTGAGTTTTTATTTACCTTTTCTTGCAGGTCTGAAGGACATTTAT
CCTGATGGCGTGATTGCGGATATTGGTTGCGGACGTGGCGAATGGTTGGAGATCCTGACTGAAAATGGCATTGCGAA
CATCGGCGTCGATCTCGATGATGGCATGCTGGCGCGCGCCAGGGAGGCCGGACTGAATGTGCAGAAAATGGATTGTC
TGCAGTTTTTGCAAAGTCAGGCGGATCAGAGCCTGATAGCGTTGACCGGTTTTCATATTGCTGAGCATTTGCCGTTT
GAGGTCCTGCAGCAACTCGCCATGCATACCCTACGGGTGCTGAAACCAGGTGGTTTGCTGATCCTCGAAACGCCGAA
CCCGGAGAATGTAAGCGTCGGCACCTGTTCATTTTATATGGATCCAACGCATAATCATCCTCTGCCACCGCCACTGC
TTGAGTTTTTACCTATTCATTATGGTTTTACCCGAGCAATTACCGTTCGTCTGCAGGAAAAAGAGGTTCTTCAATCT
CCGGATGCAGCCGTTAATTTGGTCGATGTACTCAAAGGGGTGAGCCCCGACTACAGCATCATTGCTCAGAAAGCAGC
GCCAACAGATATTCTTGAACGCTTTGACACCCTGTTTACCCAGCAGTACGGTCTGACGCTGGATGCTCTGAGCAACC
GTTACGATGCGATTTTGCGCCAACAGTTTTCGTCCGTTGTCTCACGGCTGGAGACGTTGAACCAAACCTATATGCAA
CAGATAAGCCAAATGTCAGAGACTATTCAGACGTTGCAAGGTGAGGTTGACGATCTGAGTCATGTCATCGATCAGAA
CCATCAGCTTCATCAGCAAATGGCGGATTTACATAACAGTCGTTCATGGCGTATTACTCAACCACTACGCTGGTTGT
CTTTGCAACGTCAATTATTACGTCAGGAAGGGGCTAAAGTGCGAGCCCGTAGGGCTGGGAAAAAAATATTGCGCAAA
GGGATGGCGCTCTCGCTGGTCTTTTTCCATCGTTACCCTAAGTCTAAGGTTTATCTGTTTAAGGTTCTGAGAAAAAC
TGGCTGCTATACATTGCTACAACGTTTGTTCCAACGCGTAATGCTGGTGCAATCTGACACGATGATGATGCAGTCCA
GAAGATATGATGTGGGTACTGAAGAAATGACAAGTCGCGCGATGAGTATTTATAACGAATTAAAAAATAAAAATACG
GAGAAATAACGATGCGTATTGTCATAGATTTACAAGGCGCACAGACGGAAAGCCGCTTTCGTGGCATCGGTCGTTAT
AGTATCGCAATCGCCAGAGGCATAATCAGAAATAACAGCCGGCATGAGATTTTCATCGCGCTATCCGCCATGCTGGA
TGAGTCGATTGCAAATATTAAGGCGCAATTTGCCGATCTCCTGCCGGCAGAAAATATAGTCGTATGGCATGCCGTAG
GCCCTGTTCGTGCGATGGACCAAGGTAATGAATGGCGTCGGGAGAGCGCAGAACTGATTCGGGAAGCGTTTCTTGAA
TCATTGTGTCCAGATGTCGTTTTCATTACGAGTTTGTTTGAAGGTCATGTCGACGATGCGGCTACATCGGTACACAA
ATTTAGTCGTCAGTATAAAGTAGCCGTACTGCACCACGATCTTATCCCCCTCGTGCAGGCGGAAACCTATCTGCAGG
ACGATGTATACAAACCCTACTATTTACAGAAAGTTGAGTGGTTAAAAAACGCTGACCTTTTGTTGACTAACTCTGCT
TATACCGCACAGGAAGCGATCGAGCATCTGCATTTACAGGGCGATCATGTGCAGAATATTGCAGCCGCAGTCGATTC
TCAGTTTTGTATGGCGGAGGTGGCAGCGAGCGAAAAAGAGACCGTCCTTGGCCATTACGGTATTCAGCGCGAGTTCA
TGTTGTATGCGCCCGGAGGATTTGACTCAAGGAAAAACTTTAAACGGTTGATTGAGGCCTATGCCGGGCTCAGTGAT
GCCTTACGTCGCAGTCATCAACTGGTCATCGTCAGTAAGCTTTCCATCGGTGATCGTCAGTATCTGGAATCCCTTGC
GTCAGGTAATGGTTTACAGCAGGGCGAACTGGTACTCACTGGTTATGTGCCGGAAGATGAGCTGATCCAGCTCTATC
GCCTATGTAAGCTGTTCATCTTTGCTTCACTACATGAAGGTTTTGGGTTGCCGGTTCTGGAAGCAATGTCGTGCGGT
GCGCCGGTGATTGGCTCAAATGTCACCAGTATTCCTGAAGTCATCGGTAATCCTGAGGCATTATTCGACCCGTATTC
TGTCTCTTCCATGAGGGATAAGATCGCGCAATGTTTGACTGATGATACCTTCCTCGCGCGTCTGAAAGAAATGGCGC
AGCAGCAAGCGCGTAATTTCTCTTGGGATAAAGCTGCGGTGACTGCTCTGGAAGCTTTCGAAAAGATCGCGGTAGAA
GACACCGGTACTGCGCAGGTTTTGCCTGAAGCTTTGATTCAGAAGATCCTTGCTATCTCACAAGGGCAGCCAGATGA
CCGCGATCTGCGCTTGTGCGCAACGGCCATTGATTACAATCTGAAAACGGCAGAACTTTATCAAATCGACGATAAAT
CGCTGAACTGGCGTGTGGAAGGCCCATTCGATAGCTCATATAGTCTGGCGTTGGTCAACCGCGAATTTGCCCGGGCA
CTCTCAGCCGATGGTGTAGAGGTTTTATTGCATTCCACTGAAGGACCAGGTGATTTTGCCCCAGATGCCTCGTTTAT
GGCACAGTCGGAAAATAGTGATCTTCTGGCATTTTATAATCAATGTCAGACCCGCAAGAGTAACGAAAAGATAGATA
TTATTAGCAGAAATATCTATCCACCGCGGGTTACCAAAATGGATGCCAAAGTAAAATTCCTTCATTGTTATGCTTGG
GAAGAAACGGGCTTTCCGCAACCGTGGATCAATGAATTTAATCGGGAACTTGACGGAGTGCTGTGTACTTCGGAACA
TGTTCGTAAAATACTGATTGATAACGGACTGAATGTGCCCGCATTTGTTGTTGGCAATGGCTGTGACCATTGGCTCA
ATATCCCAGCCGAGACGACAAAAGATGTGGATCACGGAACATTCCGTTTCCTGCACGTCTCTTCTTGTTTCCCACGC
AAAGGGATACAGGCAATGCTTCAGGCTTGGGGGAAGGCGTTCACTCGTCGTGACAATGTTATCTTAATCATTAAGAC
TTTTAACAATCCGCACAATGAAATTGACGCATGGCTGGCTCAGGCCCAGGCTCAATTCATAGACTATCCCAAAGTTG
AAGTGATCAAAGAGGATATGTCAGCCACCGAGCTTAAAGGGCTTTATGAAAGCTGTGATGTTTTGGTTGCTCCAGGT
TGCGCTGAAGGCTTTGGTTTACCTATTGCTGAAGCAATGCTGAGTGGGCTACCGGCTATCGTCACCAATTGGAGCGG
GCAACTTGATTTTGTTAATTCACAAAATTCATGGCTGGTTGACTATCAGTTCACTCGGGTAAAAACGCACTTTGGTC
TGTTTTCCTCAGCCTGGGCCAGTGTGGATATTGACAACTTAACAGATGCATTAAAAGCGGCAGCCTCAACCGATAAA
TCAGTGCTGCGTGACATGGCCAATGCTGGTCGCGAGCTTCTTCTGCAGCAGTTTACCTGGAAAGCGGTGGCTGATCG
TTCTTGCCAGGCGGTCAAGACTCTGCGTGCGCATATTGATATTGCACAGCATCGGGCGCGCATTGGCTGGGTGACGA
CCTGGAACACGAAATGTGGGATCGCAACCTATTCCCAGCATCTGGTGGAAAGCGCACCTCATGGCGCGGATGTTGTT
TTTGCTCCCCAGGTCAGCGCTGGCGATCTTGTGTGTGCAGACGAAGAGTTTGTACTTCGCAACTGGATTGTAGGTAA
AGAGAGCAACTATCTGGAAAACCTCCAGCCACACATTGATGCTCTGAGACTCGATGTCATTGTGATCCAATTCAACT
ATGGATTCTTTAATCATCGAGAACTGTCGGCGTTTATTCGTCGCCAGCATGACGCCGGTCGTTCAGTTGTTATGACG
ATGCACTCAACTGTGGATCCGCTGGAAAAAGAGCCGAGCTGGAATTTCCGTCTTGCTGAAATGAAAGAGGCGCTGGC
ACTTTGCGACCGGTTGTTGGTGCATTCGATTGCCGATATGAACCGCCTTAAAGATTTAGGCTTAACTGCGAATGTTG
CTTTATTCCCGCACGGTGTTATCAACTACTCCGCAGCGAGCGTCACACGTCAACAGCAGTCTTTACCGCTAATTGCG
AGCTATGGCTTCTGCTTACCGCATAAGGGCCTGATGGAACTAGTAGAATCCGTCCATAGACTCAAGCAAGCCGGTAA
ACCGGTTCGTTTACGACTGGTGAACGCAGAGTATCCTGTTGGGGAGTCACGCGATCTGGTGGCAGAGCTTAAAGCTG
CTGCTCAGCGGTTAGGTGTTACCGATCTGATTGAGATGCATAATGATTTCCTACCTGATGCGGAGAGTCTGCGGTTG
CTTTCAGAAGCCGATCTTCTGATTTTTGCTTATCAGAATACTGGGGAGTCTGCTAGCGGGGCGGTACGTTATGGTAT
GGCGACTCAAAAACCTGTTGCGGTAACGCCCCTGGCGATATTTGATGATTTGGACGATGCCGTCTTTAAATTTGATG
GATGCAGCGTCGATGATATCAGTCAGGGGATTGACCGGATCCTGAATTCCATCCGTGAACAGAACTCTTGGGCAACC
AGGACTCAACAACGTGCCGATGCATGGCGGGAACAACATGATTATCAAGCTGTTTCACGCCGTCTGGTTAATATGTG
TCAAGGCTTAGCTAAAGCTAAATATTTTAAATAAAAATATCTCTCTTGTATTTTTTGCCTTTGAATACAAGAGGGGT
TAGATAATGTGTCATTTATTATGAAAATTATTTTTGCTACTGAGCCAATTAAATACCCATTAACGGGCATCGGTCGG
TATTCCCTGGAGCTGGTTAAGCGGCTGGCGGTCGCCCGCGAAATTGAAGAATTAAAGCTATTTCACGGTGCGTCGTT
TATAGAACAGATCCCTTTGGTGGAGAATAAAAGCGATACCAAAGCCAGCAATCATGGTCGTCTGTCGGCGTTTCTAC
GCCGACAGACGCTGTTGATTGAGGCTTATCGCTTGCTGCATCCGCGGCGCCAGGCGTGGGCATTGCGCGACTATAAG
GATTATATCTACCATGGCCCCAATTTTTATCTGCCGCATAAACTGGAACGCGCCGTGACCACGTTTCATGACATATC
CATTTTTACCTGCCCGGAATATCATCCAAAAGATCGGGTTCGCTATATGGAGAAGTCCCTGCATGAGAGTCTGGATT
CGGCAAAGCTGATCCTGACCGTTTCTGATTTCTCGCGCAGTGAAATTATCCGCTTGTTCAACTATCCGGCGGAGCGG
ATCGTAACCACCAAGCTAGCCTGCAGCAGTGACTATATCCCACGCAGCCCGGCAGAGTGTCTGCCGGTACTGCAGAA
ATATCAGCTGGCGTGGCAGGCCTACGCGCTATATATCGGCACTATGGAGCCACGTAAAAATATCCGAGGCCTGCTGC
ATGCCTATCAGCTGCTACCGATGGAGATCCGCATGCGCTATCCGCTAATCCTTAGCGGCTATCGCGGCTGGGAAGAC
GATGTGCTGTGGCAGTTAGTCGAGCGCGGTACTCGGGAAGGCTGGATCCGTTACCTCGGATATGTTCCGGATGAAGA
CCTGCCGTATCTGTACGCAGCGGCCAGAGTCTTTGTTTATCCCTCCTTCTACGAGGGATTCGGTTTACCTATTCTTG
AAGCGATGTCTTGCGGTGTGCCGGTAGTATGCTCCAATGTCACCTCTTTGCCTGAGGTTGTTGGCGATGCCGGCCTC
GTTGCCGATCCTAATGATATAGACGCGATTAGCGCGCAAATTTTGCAGAGCCTGCAAGATGATAGCTGGCGGGAAAT
CGCCACCGCGCGCGGTCTTGCTCAGGCGAAACAGTTTTCGTGGGAGAACTGTGCGACACAGACCATTAACGCCTATA
AATTACTCTAAGGGTGTCAGTTGAGAGTTCTACACGTCTATAAGACTTACTATCCCGATACCTACGGCGGTATTGAG
CAGGTCATTTATCAGCTAAGTCAGGGCTGCGCCCGCCGGGGAATCGCAGCCGATGTTTTCACTTTTAGCCCGGACAA
AGATACAGGTCCTGTCGCTTACGAAGATCATCGGGTCATTTATAATAAACAGCTTTTTGAAATTGCCTCCACGCCGT
TTTCGCTGAAAGCGTTAAAGCGTTTTAAGCTGATTAAAGATGACTACGATATCATCAACTACCATTTTCCGTTTCCC
TTTATGGATATGCTGCATCTTTCGGCGCGGCCTGACGCCAGGACTGTGGTGACCTATCACTCTGATATAGTGAAACA
AAAACGGTTAATGAAGCTGTACCAGCCGCTGCAGGAGCGATTTCTCAGCGGCGTAGATTGCATCGTTGCCTCGTCGC
CCAATTACGTGGCTTCCAGCCAGACCCTGAAAAAATATCTGGATAAAACGGTGGTGATCCCGTTTGGTCTGGAGCAG
CAGGACGTGCAGCACGATCCGCAGAGGGTCGCGCACTGGCGGGAAACTGTCGGCGATAAGTTCTTTCTCTTCGTCGG
CACTTTCCGCTACTACAAAGGGCTGCATATTCTGATGGATGCCGCTGAGCGTAGCCGACTGCCAGTGGTGGTTGTAG
GGGGCGGGCCGCTGGAATCGGAAGTGCGGCGTGAAGCGCAGCAGCGCGGGCTGAGCAATGTGATGTTTACCGGCATG
CTCAACGACGAAGATAAGTACATTCTCTTCCAGCTCTGCCGGGGCGTGGTATTCCCCTCGCATCTGCGCTCTGAGGC
GTTTGGCATTACGTTATTGGAAGGCGCACGCTTTGCAAGGCCGCTGATCTCTTGCGAGATCGGTACAGGTACCTCTT
TCATTAACCAGGACAAAGTGAGTGGTTGCGTGATTCCGCCGAATGATAGCCAGGCGCTGGTGGAGGCGATGAATGAG
CTCTGGAATAACGAGGAAACCTCCAACCGCTATGGCGAAAACTCGCGTCGTCGTTTTGAAGAGATGTTTACTGCCGA
CCATATGATTGACGCCTATGTCAATCTCTACACTACATTGCTGGAAAGCAAATCCTGAGCGGCCGCGAGCTCGTCGA
CTCGAGGATCCGTGTAGGCTGGAGCTGCTTCGAAGTTCCTATACTTTCTAGAGAATAGGAACTTCGGAATAGGAACT
AAGGAGGATATTCATATGGATAAAGCCGTAAGCATATAAGCATGGATAAGCTATTTATACTTTAATAAGTACTTTGT
ATACTTATTTGCGAACATTCCAGGCCGCGAGCATTCAGCGCGGTGATCACACCTGACAGGAGTATGTAATGTCCAAG
CAACAGATCGGCGTAGTCGGTATGGCAGTGATGGGACGCAACCTTGCGCTCAACATCGAAAGCCGTGGTTATACCGT
CTCTATTTTCAACCGTTCCCGTGAGAAGACGGAAGAAGTGATTGCCGAAAATCCAGGCAAGAAACTGGTTCCTTACT
ATACGGTGAAAGAGTTTGTCGAATCTCTGGAAACGCCTCGTCGCATCCTGTTAATGGTGAAAGCAGGTGCAGGCACG
GATGCTGCTATTGATTCCCTCAAACCATATCTCGATAAAGGAGACATCATCATTGATGGTGGTAACACCTTCTTCCA
GGACACTATTCGTCGTAATCGTGAGCTTTCAGCAGAGGGCTTTAACTTCATCGGTACCGGTGTTTCTGGCGGTGAAG
AGGGGGCGCTGAAAGGTCCTTCTATTATGCCTGGTGGCCAGAAAGAAGCCTATGAATTGGTAGCACCGATCCTGACC
AAAATCGCCGCCGTAGCTGAAGACGGTGAACCATGCGTTACCTATATTGGTGCCGATGGCGCAGGTCACTATGTGAA
GATGGTTCACAACGGTATTGAATACGGCGATATGCAGCTGATTGCTGAAGCCTATTCTCTGCTTAAAGGTGGCCTGA
ACCTCACCAACGAAGAACTGGCGCAGACCTTTACCGAGTGGAATAACGGTGAACTGAGCAGTTACCTGATCGACATC
ACCAAAGATATCTTCACCAAAAAAGATGAAGACGGTAACTACCTGGTTGATGTGATCCTGGATGAAGCGGCTAACAA
AGGTACCGGTAAATGGACCAGCCAGAGCGCGCTGGATCTCGGCGAACCGCTGTCGCTGATTACCGAGTCTGTGTTTG
CACGTTATATCTCTTCTCTGAAAGATCAGCGTGTTGCCGCATCTAAAGTTCTCTCTGGTCCGCAAGCACAGCCAGCA
GGCGACAAGGCTGAGTTCATCGAAAAAGTTCGTCGTGCGCTGTATCTGGGCAAAATCGTTTCTTACGCCCAGGGCTT
CTCTCAGCTGCGTGCTGCGTCTGAAGAGTACAACTGGGATCTGAACTACGGCGAAATCGCGAAGATTTTCCGTGCTG
GCTGCATCATCCGTGCGCAGTTCCTGCAGAAAATCACCGATGCTTATGCCGAAAATCCACAGATCGCTAACCTGTTG
CTGGCTCCGTACTTCAAGCAAATTGCCGATGACTACCAGCAGGCGCTGCGTGATGTCGTTGCTTATGCAGTACAGAA
CGGTATTCCGGTTCCGACCTTCTCCGCAGCGGTTGCCTATTACGACAGCTACCGTGCTGCTGTTCTGCCTGCGAACC
TGATCCAGGCACAGCGTGACTATTTTGGTGCGCATACTTATAAGCGTATTGATAAAGAAGGTGTGTTCCATACCGAA
TGGCTGGATTAA
(example O15 rfb locus nucleotide sequence-O15-EPA production strain
stLMTB11738)
SEQ ID NO: 15
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGCTAGCATGAGCAAAACTAAACTAAATGTTCTTTACCTTGCAA
TAAGTCAGGGTGCCAATTACCTACTGCCATTATTAATTTTTCCTTATCTTGTTAGAGTCATTGGTGTATCGAATTTT
GGTGATCTGAGTTTTTCATTGATAACTATACAAGTGTTGTTAATGGTTGTTGAATATGGTTTTGGATATAGTGGGAC
AAGAGAAATAGCACTAAATAACGATAAAAAATACCATTCTGAATTTTTTTGCGGTGTGGTGCTTGCTCGTTTTATAT
TAATGCTAATTGCAGCTATAATACTCATAATACTCTGTTTTTTTTATGTTTTTAACGACGTTAAGTCTTTGTTATGT
GTTGGTTTTCTGTCCGTAATTGCAGGTGTTTTCAATCCAAATTGGTTTTTGCAAGGTAAGGAAATGATGAGTGTGAT
GGCTGTGCTGTCACTATTTTCACGAGGCATAGCAGTCGTTGCAGTTTATCTAATTATAAAACCCGCAACGCCGATGT
ACATCAGTGCCTTATTATTGAGCATGCCATATATTTTGTATTCATTCTGTGGCGTTGCCTACTTACTTATTATCAAG
GAGATTTTTTTATGTAGGCCACCGATAAAGAAAATTCAAGTAATTTTAAAAAATGGATTTCATTTTTTTTGTTCAAC
ACTTGCGACTAGTGCATACACAATGTTGACCCCTCTTGTATTGGGTGGCGTATCTGGAAAGTTTGATGTAGGCATCT
TTAACTCAGCTAACATGATCAAACAAGGTTTGGCTGGACTTGCATCACCATTAGTCCAAGCTTTTTATCCAAGAATT
AACATTTTGCAAAGAGAGAATCCATATATTGCAAACTTAAAATCTAGAATGATTCTTAAATACTTGCTTGTTTTTTA
CATGGCTTTAGCAATACCATTTTTACTTTTTGCCAACCAATTATCATTATTAATATTCGGCATGAAAGGTGAAGTAA
TTGCAGGTGCAATGCAATTAATGACATTGCTTCCTATATTCATAGGTTTTAATACAGTTGTCGGGTTACTTGTATTA
GTACCTAATGGGATGCAAAAACAGTATTTCAAATCTATTTTCCTAGGAACTATTACTTGTTTAAGCATAGTTTATCC
AGCATGTAAATATTATGGAGCAACGGGTGCGATTGTGAGTCTTATTGTAGCTGAAATTTTCGTTGGCATGGGAATGC
TTAAACAATTCATTAAAGTAAATAAAACCGTATGTAGGCCTCATAAATTATGAATATCTCGGTAATAATATCTGTTT
GGAAACGCCCAGTTCAATTAGAATTGATTCTCTCTGAGCTCGATTCTCAGGCTAAAGACAATAGTCTACACCTAGAA
GTAATTGTTTCCGATAGTCATAGTGGTAAAGAAATTGATGATGTAGTTGCTGATAATATTCATAAAAAGAAAAATAT
TAATATTATCCATCAACATACTAAAAATATACTCTCCGCTAAGCGCAATTTCGGAGCATCCCTAGCCCATGGGGATT
ATTTAATATTTCTTGATGATGATTGTATACCCGCAAGTGGATATATATCATCGTTGCTGAACTATTTAAAAAAAATG
AATAGTAAAAGCGTTTTATGTGGGGAAGTTAGATTCGAAAATGAACTCATTGAGACCAGCAATTACTATCGCTACAG
GAACTCTTTACACCCTAAGTTTAGTGATAGTCCTGATATCTCTATGAATGCCTGGACTTTTGTCGCAATGAATTGTG
TTCTTGATAGAAAGGCATTTTCATCAGGTATAGTTTCATATAATGAAAATTTTATTGGTTATGGTTGTGAAGATCAT
GAGTTTGGGTGGCAACTTGAAAAAAATGACTTCAAAATTATTTTTGCTGATTTTAAAATATTACATCACGAATACAG
TGGCGATATAGAAGGATATACAAAAAAAATTCGTGCTACAGCACGTGATGGTATGAATGTATTAAGCAAAGTAAGGC
CTGAAATGTTTTCTACTAATAAAAAATTATTCCTAGTTGAGAAAATATTTAGTAAACACAAAACGTTTAGTAAAATA
TGCCAATCAATATTTTTCAATAAATTTATTTTTAAAAAAATAATACAATTTTTAAAAAAAACAGATGCAAATAAAAA
ACTCTATTTCCCAATTCTTTACAGATATGTGTTGATTTCGGCATATATACATGGTATTGGAGAGCGTGGCACCTCAA
AAACAGATGATTTGCTTAAGAACTGGTATATATAGATGATGCTATCTTCATTTATTAAGACATTTGTATGGAAGGTA
AAAAACAATGAAGTATAATGCATTGATGGCTTTTTTATTATTTTTTGTTGTTTTTTTTAGATTGTCGCTGATAATAC
CTTTCTTATATTTGGCATTTATTCCTGCATTTTTTGGTATTATGTATTTAGTGCGTAATTTTATGATTACTATGGGC
AATGGATTGGTATCTATAGATCGTAAAAATTTGTTGCTGTTATCTATATTCATAATTATTTTTTTATTTTGTTTGGT
TTTCGATTTGTTTCAAAAAAGCCATTCTTTTCAAAGTTATTTTACCGTTAGATTATTTATGTTGTTTTTATTTTCAT
TTGTTCCTGCGTATTATTTAGTAAATAGATTCATAAAGGGTGACTTGAAATTAATGGAGCGAATATTAGTGTATTCT
CTCTGGGTTCAAATAGTTATTTTTTTTGGTATGTATATAAGTCCAGAGTTAAAAAGATTGTTATATACTTTCTTTGG
TATGTCTGACTCTGTTAATCTTTGGGAACAAAATGCTAAAGTAAGAGGATTTGGGTTGTCGGGTGAAATAAATTTCA
TGACACCATTTTTGATGATCTATATGTCATTTTTTATGATGAAAAGGCGTTATGCTTTAATTACTTTAATTTGTCTG
ACTCAAATCGTAAATTCTAACATGGCTGTGATTGCAGCCATTATTGGTATCGGTTGCTCTAGACTTAATATTAATAT
AAAAATTGCAACAGTATTGATTTTGGGAGTTTTAGTTTATAGCTTAGGAGCGGTGTTCTTTCCTCGATTTTATGATG
AGTTCGTTTCTGGAGATGGCACAAGAACTCTGGATATCTTATTACAGCAACATGTGTTTGTTGTAGGTAATTTAGAT
TTTTTTAATATTATATTTGGATTACAGCAAAACATATCTTCATCAATCCCCGATATTAAACAAAGTTCGGATATGGG
CTGGGTTATACTGTTTAATTACGGTGGGTTAACATTTATTACACTCTTTTTATTTTTAATCTTTACTATTTCTATTG
CGACATTTGGAATGACATATCAAGCAATTATATGGATGTTAATTGGGATAATTTTCAATACCAAAGGTTTAGTTTTA
GGATCTAACGGCTATTTCTTTCTATCTTTTATATATATGTTTTTGAATAGAGTAACACTTAGTGGACAGAGTTCAAT
TACTAATAAGTTAGGTCAAGTAAGTAAATAGCTTCCAGAGTATATTTGTCAATGATTTGAGGTTCGGTTATTATGTT
TTCATCTAAAACACTGTTAATTACTGGTGGTACTGGCTCTTTCGGGAATGCTGTATTAAATAGATTTCTTGATACAG
ATATTGCAGAAATCCGTATATTTAGTCGTGATGAAAAAAAACAAGATGATATGCGGAAAAAATACAATAATCAAAAA
TTAAAGTTCTATATTGGTGATGTCAGAGATTACCGTAGTATTTTGAATGCGACTCGCGGTGTTGATTTTATATATCA
TGCAGCGGCACTTAAGCAAGTTCCATCATGTGAATTTCATCCTATGGAAGCCGTTAAAACTAATATCCTTGGTACGG
AAAATGTTCTTGAAGCAGCTATAGCGAATGAAGTGAAGAGGGTTGTATGCCTAAGTACTGATAAAGCTGTATACCCG
ATTAACGCAATGGGTATTTCAAAAGCTATGATGGAAAAGGTCATGGTCGCGAAATCCCGTAATGTTGATCGCAATAA
AACAGTAATATGTGGTACCCGTTATGGGAATGTTATGGCATCTCGCGGTTCAGTTATTCCATTATTTGTTGATCTTA
TTAGAGCGGGCAAGCCACTCACAATAACTGATCCTAATATGACCCGCTTTATGATGACTCTTGAGGATGCGGTAGAT
TTAGTTCTTTATGCGTTTGAACATGGTAATAATGGTGATATCTTTGTGCAAAAAGCACCTGCAGCAACTATTGACAC
ATTAGCTATTGCTTTAAAGGAATTACTAAATGTTCCTGACCATCCGGTAAATGTCATTGGAACGCGTCATGGCGAGA
AATTATATGAAGCTCTACTTAGTCGTGAGGAAATGATCGCTGCTATAGATATGGGCGATTATTACCGTGTCCCGCCA
GATCTTCGTGACCTTAATTATGGCAAATATGTTGAGCAAGGTGATAGCCGAATATCTGAAATAGAAGATTATAACTC
TCATAATACTCAACGGTTAGATGTTGAAGGCATGAAAGAGCTCTTGCTAAAATTAGCCTTTATTCGAGCAATTCGTG
CTGGTGAAAAATATAATCTGGATTCATGATATGAAAATATTAGTTACTGGTGCAAATGGTTTTATTGGTCGTAATTT
ATGTTTGAGGCTTGAGGAACTTGGTTATAAAGATCTTATTAGAATTGATCGAGAATCAACGAAGCAAGATCTTGAAC
AAGGCTTACAGGATGCCGATTTTATTTATCACTTAGCTGGTATCAATAGACCTAAGACTGATGATGAGTTTATTTCT
GGAAACAGTGATTTAACAAAGCATATAGTTGAGTATCTCCTTTCTATTGGTAAGAATACACCAATTATGCTAAGTTC
TTCGATACAAGCTGAACTTAATAATGCTTATGGGGTTAGCAAAGCTGTAGCTGAAAGCTATGTCGAAAAATATGCTG
CTGCTAGTGGTTCTTCGTATTATATTTTCAGATATCCAAACGTTTTTGGTAAATGGTGTAAGCCAAACTATAATTCT
TTTATAGCAACTTTTTGCTACAATATTTCCAATGATATTGAGATTACTATCAATGATGCAGCAGCGCCAGTCAATCT
GGTCTATATTGATGATGTTTGTACTGATGCTATAGCTCTTCTCTCTGGGACGGTTGAAAGTGGATATAAAGTTGTTG
CACCAATTTATTCAACAACAGTTGGTGAAGTTGCAGAATTAATTTATAGCTTCAAAAATAGCCGTTCCACCCTGATC
ACAGAGGCTGTCGGGGCGGGATTTACCCGTGCATTGTATTCTACATGGCTGAGTTATTTACCAGCAGAGAAGTTTGC
GTACAAGGTACCTTTTTATGGGGATGCCCGCGGAGTCTTTTGTGAGATGTTGAAAACGCCTTCAGCGGGGCAGTTTT
CATTTTTTACTGCTCACCCTGGTATTACGCGTGGCGGACATTACCATCACAGTAAAAATGAGAAGTTTTTGGTCATT
CGAGGTCAGGCATGCTTTAAATTTGAACATGTGATTACCGGTGAGCGATATGAACTGAAAGTTTCATCGGGTGAGTT
TAAGATTGTTGAAACAGTTCCTGGTTGGACACATGACATTACAAATATTGGAACTGATGAATTAATAGTCATGCTCT
GGGCAAATGAAATTTTCAACCGTGATGAGCCCGATACTATTGCGAGACCTCTATAATGAAAAAATTAAAAGTTATGT
CTGTTGTTGGAACCCGTCCTGAGATTATCCGTTTGTCGAGGGTTCTTGCTAAGTTTGATGAATACTGCGAGCATATT
ATTGTCCATACTGGTCAAAATTATGATTACGAATTAAATGAAGTGTTCTTCAATGACTTGGGTGTTCGAAAACCTGA
TTATTTTTTAAATGCAGCGGGTAAAAATGCGGCGGAAACCATTGGTCAGGTTATTATTAAGGTAGATGAAGTATTAG
AAATCGAAAAACCTGAAGCAATACTGGTATTGGGCGATACGAATTCATGTATTTCTGCCATTCCGGCCAAACGCCGT
AAAGTGCCTATATTTCATATGGAAGCAGGTAACCGTTGTTTCGATCAACGCGTGCCTGAAGAAACCAACAGACGTAT
TGTTGACCATACGGCTGATATCAATATGACCTACAGTGATATTGCTCGTGAATATCTCTTGGCTGAAGGTATCCCAG
CTGATCGGATCATAAAAACTGGTAGCCCTATGTTTGAGGTTCTTTCATATTATATGCCCCAAATTGATGGTTCAGAT
GTGCTATCGCGTTTGAATCTACAGTCTGGTGAGTTTTTTGTAGTAAGTGCGCATCGTGAAGAGAATGTTGATTCTCC
AAAACAGCTCGTAAAGCTTGCGAACATTCTAAATACTGTTGCTGAAAAATATAATCTTCCAGTTATTGTCTCCACAC
ACCCAAGGACACGTAACCGAATCCGTGAGCAAGGAATTGAATTTCATTCAAATATAAATCTACTGAAACCATTGGGT
TTCCATGATTATAACCACTTGCAGAAGAACTCACGAGCTGTGCTTTCAGATAGCGGTACTATCACTGAAGAGTCATC
CATCATGAATTTCCCAGCGGTAAACATCCGGGAAGCGCATGAGCGTCCGGAAGGCTTTGAGGAAGCATCCGTCATGA
TGGTGGGGTTAGAGTGTGAACGCGTATTACAAGCGCTGGATATTCTGGCAACACAACCGCGAGGTGAAGTCCGTCTT
TTACGTCAGGTTAGTGATTACAGCATGCCAAATGTGTCGGATAAAGTTGTCAGAATTGTTCACTCTTACACAGATTA
TGTTAAGAGAGTCGTCTGGAAAGAATATTGATGAAACTTGCTTTAATCATAGATGATTACCTGCCCAACAGTACTCG
TGTTGGTGCAAAAATGTTTCATGAACTTGCTCAAGAATTTATCCAGCGTGGGCACGATGTTACGGTAATTACTCCTG
GTACGGGCATGCAAGAAGAGATTTCTTTTGATACCTTTCAGGGGGTAAAAACATGGCGTTTTAAAAGCGGGCCGCTC
AAGGATGTAAGTAAAATTCAGCGAGCGGTCAATGAAACGCTTTTGTCCTATCGGGCGTGGAAAGCCATCAAAAAATG
GGTAAAAAAAGAGACCTTTGAGGGGGTGATTTATTATTCACCTTCCATATTCTGGGGGCCTTTAGTTAAAAAAATTA
AAGCTCGTTGCCAATGTCCTGCTTATCTTATTTTAAGAGATATGTTTCCACAATGGGTAATTGATGCAGGAATGCTT
AATGCTGGTTCCCCAATAGAACGCTACTTTCGTCTTTTTGAAAAAATATCTTATCGTCAGGCAAATCGTATTGGACT
TATGTCTGATAAGAATCTTGATGTTTTTCGGAAAGATAATAAAGGCTATCCGTGCGAAGTTTTGCGTAATTGGGCAT
CCCTAACACCAACGATCATACCCAAGGATTATATACCACTACGTAAGCGACTTGGCCTAGAGGATAAAACCATTTTC
TTCTATGGTGGAAACATAGGTCATGCACAGGACATGACAAACTTGATGCGACTTGTGAGAAACATGGCAGCATATCC
TCAAGCTCATTTCCTATTTATTGGCCAGGGGGATGAAGTTGAATTAATTAATTCATTAGCATCTGAGTGGGCATTGA
CGAATTTCACCTATTTGCCCTCGGTTAACCAAGATGAATTTAAGTTCATTTTGTCGGAAATGGATATCGGCTTGTTT
TCTCTTTCCGCTAGACACTCTTCCCATAATTTTCCTGGTAAGTTATTAGGCTATATGGTTCAGTCGCTACCTATTTT
AGGTAGCGTAAATGCCGGAAATGATTTGCTCGACATTGTCAATCAAAATAATGCGGGATTAATCCATGTCAATGGTG
AGGACGATAAATTATGTCAATCTGCGCTATTAATGTTGCATGATATTGATGTGCGCCGGCAACTTGGTTCGGGGGCG
AATATATTGTTGAAAGAACAATTCTCCGTTGAGTCTGCGGCACAGACGATAGAAATGAGGTTGGAGGCATGCAATGC
GATTAATTGATAATGACCAACTCGACGAATTATATGATCAAGCCGGGCAATCGGAACGTTTACGTTCCCACCTTATG
ATGCACGGCTCGCATCAAGAAAAGGTACAGCGTTTACTTATTGCATTAGTAAAGGGCAGCTATGTTGAACCGCATTA
TCACGAACTTCCTCATCAGTGGGAAATGTTCATTGTTATGGAGGGGCAACTTCAGGTTTGTTTGTATGGTAGAAATG
GTGAGGTTATAAAGCAATTTATAGCAGGAGATAATACTGGAATGAGCATTGTGGAGTTTTCTCCGGGCGATATACAC
AGTGTCGAATGCCTATCTCCGCGTGCTCTTATGGTGGAAGTTAAGGAGGGGCCATTTGACCCTTCTTTTGCAAAATC
GTTCGTGTGAGCGGCCGCGAGCTCGTCGACTCGAGGATCCGTGTAGGCTGGAGCTGCTTCGAAGTTCCTATACTTTC
TAGAGAATAGGAACTTCGGAATAGGAACTAAGGAGGATATTCATATGGATAAAGCCGTAAGCATATAAGCATGGATA
AGCTATTTATACTTTAATAAGTACTTTGTATACTTATTTGCGAACATTCCAGGCCGCGAGCATTCAGCGCGGTGATC
ACACCTGACAGGAGTATGTAATGTCCAAGCAACAGATCGGCGTAGTCGGTATGGCAGTGATGGGACGCAACCTTGCG
CTCAACATCGAAAGCCGTGGTTATACCGTCTCTATTTTCAACCGTTCCCGTGAGAAGACGGAAGAAGTGATTGCCGA
AAATCCAGGCAAGAAACTGGTTCCTTACTATACGGTGAAAGAGTTTGTCGAATCTCTGGAAACGCCTCGTCGCATCC
TGTTAATGGTGAAAGCAGGTGCAGGCACGGATGCTGCTATTGATTCCCTCAAACCATATCTCGATAAAGGAGACATC
ATCATTGATGGTGGTAACACCTTCTTCCAGGACACTATTCGTCGTAATCGTGAGCTTTCAGCAGAGGGCTTTAACTT
CATCGGTACCGGTGTTTCTGGCGGTGAAGAGGGGGCGCTGAAAGGTCCTTCTATTATGCCTGGTGGCCAGAAAGAAG
CCTATGAATTGGTAGCACCGATCCTGACCAAAATCGCCGCCGTAGCTGAAGACGGTGAACCATGCGTTACCTATATT
GGTGCCGATGGCGCAGGTCACTATGTGAAGATGGTTCACAACGGTATTGAATACGGCGATATGCAGCTGATTGCTGA
AGCCTATTCTCTGCTTAAAGGTGGCCTGAACCTCACCAACGAAGAACTGGCGCAGACCTTTACCGAGTGGAATAACG
GTGAACTGAGCAGTTACCTGATCGACATCACCAAAGATATCTTCACCAAAAAAGATGAAGACGGTAACTACCTGGTT
GATGTGATCCTGGATGAAGCGGCTAACAAAGGTACCGGTAAATGGACCAGCCAGAGCGCGCTGGATCTCGGCGAACC
GCTGTCGCTGATTACCGAGTCTGTGTTTGCACGTTATATCTCTTCTCTGAAAGATCAGCGTGTTGCCGCATCTAAAG
TTCTCTCTGGTCCGCAAGCACAGCCAGCAGGCGACAAGGCTGAGTTCATCGAAAAAGTTCGTCGTGCGCTGTATCTG
GGCAAAATCGTTTCTTACGCCCAGGGCTTCTCTCAGCTGCGTGCTGCGTCTGAAGAGTACAACTGGGATCTGAACTA
CGGCGAAATCGCGAAGATTTTCCGTGCTGGCTGCATCATCCGTGCGCAGTTCCTGCAGAAAATCACCGATGCTTATG
CCGAAAATCCACAGATCGCTAACCTGTTGCTGGCTCCGTACTTCAAGCAAATTGCCGATGACTACCAGCAGGCGCTG
CGTGATGTCGTTGCTTATGCAGTACAGAACGGTATTCCGGTTCCGACCTTCTCCGCAGCGGTTGCCTATTACGACAG
CTACCGTGCTGCTGTTCTGCCTGCGAACCTGATCCAGGCACAGCGTGACTATTTTGGTGCGCATACTTATAAGCGTA
TTGATAAAGAAGGTGTGTTCCATACCGAATGGCTGGATTAA
(example O16 rfb locus nucleotide sequence-O16-EPA production strain
stLMTB11739)
SEQ ID NO: 16
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGTGAAAATACTTGTTACTGGTGGCGCAGGATTTATTGGTTCAG
CTGTAGTTCGTCACATTATAAATAATACGCAGGATAGTGTTGTTAATGTCGATAAATTAACGTACGCCGGAAACCGG
GAATCACTTGCTGATGTTTCTGATTCTGAACGCTATGTTTTTGAACATGCGGATATTTGCGATGCACCTGCAATGGC
ACGGATTTTTGCTCAGCATCAGCCGGATGCAGTGATGCACCTGGCTGCTGAAAGCCATGTTGACCGTTCAATTACAG
GCCCTGCGGCATTTATTGAAACCAATATTGTTGGTACTTATGTCCTTTTGGAAGCCGCTCGCAATTACTGGTCTGCT
CTTGATAGCGACAAGAAAAATAGCTTCCGTTTTCATCATATTTCTACTGACGAAGTCTATGGTGATTTGCCTCATCC
AGATGAAGTAAATAATACAGAAGAATTACCCTTATTTACTGAGACGACAGCTTACGCGCCAAGCAGCCCTTATTCCG
CATCCAAAGCATCCAGCGATCATTTAGTCCGCGCGTGGAAACGTACATATGGTTTACCGACAATTGTGACTAATTGC
TCGAACAACTATGGTCCTTATCATTTCCCGGAAAAGCTTATTCCACTGGTTATTCTTAATGCACTGGAAGGTAAGGC
ATTACCTATTTATGGCAAAGGAGATCAGATCCGCGACTGGTTGTATGTTGAAGATCATGCGCGTGCGTTATATACCG
TCGTAACCGAAGGTAAAGCGGGTGAAACTTATAACATTGGTGGGCACAACGAAAAGAAAAACATCGATGTAGTGCTC
ACTATTTGTGATTTGCTGGATGAGATTGTACCGAAAGAGAAATCTTATCGTGAGCAAATCACTTATGTTGCTGATCG
TCCGGGACACGATCGCCGCTATGCTATTGATGCTGAGAAGATTGGTCGCGCATTGGGATGGAAACCACAGGAAACGT
TTGAGAGCGGGATTCGTAAAACGGTGGAATGGTACCTGTCCAATACAAAATGGGTTGATAATGTGAAAAGTGGTGCC
TATCAATCGTGGATTGAACAGAACTATGAGGGCCGCCAGTAATGAATATCCTCCTTTTTGGCAAAACAGGGCAGGTA
GGTTGGGAACTACAGCGTGCTCTGGCACCTTTGGGTAATTTGATTGCTTTTGATGTTCACTCTACTGATTATTGCGG
TGATTTTAGTAATCCTGAAGGTGTAGCTGAAACCGTAAGAAGCATTCGGCCGGATATTATTGTCAATGCAGCCGCTC
ACACCGCAGTAGACAAAGCAGAATCAGAACCGGAGTTTGCACAATTAATTAACGCAACAAGTGTCGAAGCGATTGCG
AAAGCAGCAAATGAAGTTGGAGCCTGGGTTATCCATTACTCGACTGATTACGTCTTCCCTGGAAATGGCGATATGCC
ATGGCTGGAGACGGATGCAACCGCACCACTAAATGTTTACGGTGAAACCAAGTTAGCCGGAGAAAAAGCGTTACAGG
AATATTGCGCGAAGCATCTTATTTTCCGGACCAGCTGGGTCTATGCAGGAAAAGGAAATAACTTCGCCAAAACGATG
TTACGTCTGGCAAAAGAGCGTGAAGAATTAGCGGTTATTAACGATCAGTTTGGTGCGCCAACAGGTGCTGAACTGCT
GGCTGATTGTACAGCACATGCCATTCGTGTCGCACTGAATAAACCGGATGTCGCAGGCTTGTACCATTTGGTAGCCA
GTGGTACCACAACCTGGTACGATTATGCTGCGCTGGTTTTTGAAGAGGCGCGCAAAGCAGGCATTCCCCTTGCACTC
AACAAGCTCAACGCAGTACCAACAACAGCCTATCCTACACCAGCTCGTCGTCCACATAACTCTCGCCTTAATACAGA
AAAATTTCAGCAGAACTTTGCGCTTGTCTTGCCTGACTGGCAGGTTGGCGTGAAACGAATGCTCAATGAATTATTTA
CGACTACAGCAATTTAATAGTTTTTGCATCTTGTTCGTGATGGTGGAGCAAGATGAATTAAAAGGAATGATGAAATG
AAAATGCGTAAAGGTATTATTTTAGCGGGTGGTTCTGGTACACGTCTTTATCCTGTGACTATGGCTGTCAGTAAACA
GCTATTACCTATTTATGATAAACCGATGATCTATTACCCGCTCTCTACACTGATGTTGGCGGGTATTCGCGATATTT
TGATTATCAGTACACCTCAGGATACTCCTCGTTTTCAACAATTGCTGGGTGACGGTAGCCAGTGGGGCCTGAATCTT
CAGTACAAAGTGCAACCTAGCCCAGATGGCCTCGCGCAGGCATTTATCATCGGTGAAGAGTTTATTGGTGGTGATGA
TTGTGCTTTGGTTCTTGGTGATAATATCTTTTACGGTCACGATCTGCCGAAGCTAATGGAGGCCGCTGTTAACAAAG
AAAGTGGTGCAACGGTATTTGCCTATCACGTTAATGATCCAGAACGCTATGGTGTCGTTGAGTTTGATAAAAACGGT
ACGGCAATCAGTCTGGAAGAAAAACCGTTAGAACCAAAGAGTAATTACGCCGTTACAGGTCTGTACTTTTATGATAA
CGACGTGGTTCAGATGGCGAAAAACTTGAAGCCGTCTGCACGTGGTGAGTTAGAAATTACAGATATTAACCGTATTT
ATCTTGAGCAGGGACGTCTGTCTGTCGCGATGATGGGGCGTGGCTACGCGTGGCTGGACACGGGGACTCATCAGAGT
CTGATAGAAGCAAGTAATTTTATTGCGACAATTGAAGAGCGCCAGGGATTGAAGGTTTCCTGTCCTGAAGAGATTGC
ATTTCGTAAAGGTTTTATTGATGTTGAGCAAGTAAGAAAATTAGCTGTACCACTAATAAAGAATAATTATGGGCAGT
ATCTTTATAAAATGACGAAGGATTCAAATTAATGAATGTGATTAGAACTGAAATTGAAGATGTGCTAATTCTGGAGC
CAAGAGTATTTGGTGATGATAGAGGTTTCTTTTATGAGAGCTTTAATCAATCAGCATTTGAACATATTCTAGGCTAT
CCGGTCAGCTTTGTTCAAGACAATCACTCACGTTCATCAAAAAATGTACTCAGAGGCCTTCACTTTCAACGCGGCGA
GTACGCACAAGATAAACTTGTACGCTGCACTCATGGAGCAGTTTTTGATGTTGCTGTTGATATTCGACCCAATTCGG
TATCCTTTGGTAAATGGGTTGGTGTTCTGCTTTCAGCTGATAATAAGCAGCAGTTGTGGATACCAAAAGGGTTTGCT
CATGGCTTTTTGGTTCTGTCTGATATCGCTGAATTTCAATATAAAACTACAAACTATTATCATCCTGAAAGCGATTG
TGGAATATGTTGGAATGATGAACGCATTGCAATTGATTGGCCCCAAACATCAGGGTTAATCCTTTCGCCAAAAGATG
AAAGGCTCTTTACGTTAGATGAGCTTATCAGATTAAAATTAATTGCATGAATACGAATAAATTATCTTTAAGAAGAA
ACGTTATATATCTGGCTGTCGTTCAAGGTAGCAATTATCTTTTACCATTGCTTACATTTCCATATCTTGTAAGAACA
CTTGGTCCTGAAAATTTCGGTATATTCGGTTTTTGCCAAGCGACTATGCTATATATGATAATGTTTGTTGAATATGG
TTTCAATCTCACAGCAACTCAGAGTATTGCCAAAGCAGCAGATAGTAAAGATAAAGTAACGTCTATTTTTTGGGCGG
TGATATTTTCAAAAATAGTTCTTATCGTCATTACATTGATTTTCTTAACGTCGATGACCTTGCTTGTTCCTGAATAT
AACAAGCATGCCGTAATTATATGGTCGTTTGTTCCTGCATTAGTCGGGAATTTAATCTACCCTATCTGGCTGTTTCA
GGGAAAAGAAAAAATGAAATGGCTGACTTTAAGTAGTATTTTATCCCGCTTGGCTATTATCCCTCTAACATTTATTT
TTGTGAACACAAAGTCAGATATAGCAATTGCCGGTTTTATTCAGTCAAGTGCAAATCTGGTTGCTGGAATTATTGCA
CTAGCTATCGTTGTTCATGAAGGTTGGATTGGTAAAGTTACGCTATCATTACATAATGTGCGTCGATCTTTAGCAGA
CGGTTTTCATGTTTTTATTTCCACATCTGCTATTAGTTTATATTCTACGGGAATAGTTATTATCCTGGGATTTATAT
CTGGACCAACGTCCGTAGGGAATTTTAATGCGGCCAATACTATAAGAAACGCGCTTCAAGGGCTATTAAATCCTATC
ACCCAAGCAATATACCCAAGAATATCAAGTACGCTTGTTCTTAATCGTGTGAAGGGTGTGATTTTAATTAAAAAATC
ATTGACCTGCTTGAGTTTGATTGGTGGTGCTTTTTCATTAATTCTGCTCTTGGGTGCATCTATACTAGTAAAAATAA
GTATAGGGCCGGGATATGATAATGCAGTGATTGTGCTAATGATTATATCGCCTCTGCCTTTTCTTATTTCATTAAGT
AATGTCTATGGCATTCAAGTTATGCTGACCCATAATTATAAGAAAGAATTCAGTAAGATTTTAATCGCTGCGGGTTT
GTTGAGTTTGTTGTTGATTTTTCCGCTAACAACTCTTTTTAAAGAGATTGGTGCAGCAATAACATTGCTTGCAACAG
AGTGCTTAGTTACGTCACTCATGCTGATGTTCGTAAGAAATAATAAATTACTGGTTTGCTGAGGATTTTATGTACGA
TTATATCATTGTTGGTTCTGGTTTGTTTGGTGCCGTTTGTGCGAATGAGTTAAAAAAGCTAAACAAAAAAGTTTTAG
TGATTGAGAAAAGAAATCATATCGGTGGAAATGCGTACACAGAGGACTGTGAGGGTATCCAGATTCATAAATATGGT
GCACATATTTTTCATACCAATGATAAATATATATGGGATTACGTTAATGATTTAGTAGAATTTAATCGTTTTACTAA
TTCTCCACTGGCGATTTATAAAGACAAATTATTCAACCTTCCTTTTAATATGAATACTTTCCACCAAATGTGGGGAG
TTAAAGATCCTCAAGAAGCTCAAAATATCATTAATGCTCAGAAAAAAAAGTATGGTGACAAGGTACCTGAAAATTTG
GAGGAGCAGGCGATTTCATTAGTTGGGGAGGACTTATACCAAGCATTGATAAAGGGTTATACGGAGAAGCAGTGGGG
AAGAAGTGCAAAAGAATTGCCTGCATTTATTATTAAGCGAATCCCAGTGAGATTTACGTTTGATAACAATTATTTTT
CCGATCGCTATCAAGGTATTCCGGTGGGAGGCTACACTAAGCTTATTGAAAAAATGCTTGAAGGTGTGGACGTAAAA
TTAGGCATTGATTTTTTGAAAGACAAAGATTCTCTAGCGAGTAAAGCCCATAGAATCATCTACACTGGACCCATTGA
TCAGTACTTCGACTATAGGTTTGGAGCGTTAGAATATCGCTCTTTAAAATTTGAGACGGAACGCCATGAATTTCCAA
ACTTCCAAGGGAATGCAGTAATAAATTTCACTGATGCTAATGTACCATATACCAGAATAATTGAGCATAAACATTTT
GACTATGTTGAGACAAAGCATACGGTTGTTACAAAAGAATATCCATTAGAGTGGAAAGTTGGCGACGAACCCTACTA
TCCAGTTAATGATAATAAAAACATGGAGCTTTTTAAGAAATATAGAGAGTTAGCTAGCAGAGAAGACAAGGTTATAT
TTGGCGGGCGTTTGGCCGAGTATAAATATTATGATATGCATCAAGTGATATCTGCCGCTCTTTATCAAGTGAAAAAT
ATAATGAGTACGGATTAATGATCTATCTTGTAATTAGTGTCTTTCTCATTACAGCATTTATCTGTTTATATCTTAAG
AAGGATATATTTTATCCAGCCGTATGCGTTAATATCATCTTCGCACTGGTCTTATTGGGATATGAAATAACGTCAGA
TATATATGCTTTTCAGTTAAATGACGCTACGTTGATTTTTCTACTTTGCAATGTTTTGACATTTACCCTGTCATGTT
TATTGACGGAAAGTGTATTAGATCTAAATATCAGAAAAGTCAATAATGCTATTTATAGCATACCATCGAAGAAAGTG
CATAATGTAGGCTTGTTAGTTATTTCTTTTTCGATGATATATATATGCATGAGGTTAAGTAACTACCAGTTCGGGAC
TAGCTTACTTAGCTATATGAATTTGATAAGAGATGCTGATGTTGAAGACACATCAAGAAATTTCTCAGCATACATGC
AGCCAATCATTCTAACTACTTTTGCTTTATTTATTTGGTCTAAAAAATTTACTAATACAAAGGTAAGTAAAACATTT
ACTTTACTTGTTTTTATTGTATTCATCTTTGCAATTATACTGAATACTGGTAAGCAAATTGTCTTTATGGTTATCAT
CTCTTATGCATTCATCGTAGGTGTTAATAGAGTAAAACATTATGTTTATCTTATTACAGCTGTAGGTGTTCTATTCT
CCTTGTATATGCTCTTTTTACGTGGACTGCCTGGGGGGATGGCATATTATCTATCCATGTATTTGGTCAGCCCTATA
ATCGCGTTTCAGGAGTTTTATTTTCAGCAAGTATCTAACTCTGCCAGTTCTCATGTCTTTTGGTTTTTTGAAAGGCT
GATGGGGCTATTAACAGGTGGAGTCTCTATGTCGTTGCATAAAGAATTTGTGTGGGTGGGTTTGCCAACAAATGTTT
ATACTGCTTTTTCGGATTATGTTTATATTTCCGCGGAGCTAAGCTATTTGATGATGGTTATTCATGGCTGTATTTCA
GGTGTTTTATGGAGATTGTCTCGAAATTACATATCTGTGAAAATATTTTATTCATATTTTATTTATACCTTTTCTTT
CATTTTTTATCATGAAAGCTTCATGACTAATATTAGCAGTTGGATACAAATAACTCTTTGTATCATAGTATTCTCTC
AATTTCTTAAGGCCCAGAAAATAAAGTGAAAATGTATTTTTTGAATGATTTAAATTTCTCTAGACGCGATGCTGGAT
TTAAAGCAAGAAAAGATGCACTGGACATTGCTTCAGATTATGAAAACATTTCTGTTGTTAACATTCCTCTATGGGGT
GGAGTAGTCCAGAGAATTATTAGTTCTGTTAAGCTTAGTACATTTCTCTGCGGTCTTGAAAATAAAGATGTTTTAAT
TTTCAATTTCCCGATGGCCAAACCATTTTGGCATATATTGTCATTCTTTCACCGCCTTCTAAAATTTAGAATAGTAC
CTCTGATTCATGATATTGATGAATTAAGAGGAGGAGGGGGTAGTGATTCTGTGCGGCTTGCTACCTGTGATATGGTC
ATAAGTCACAATCCACAAATGACAAAGTACCTTAGTAAATATATGTCTCAGGATAAAATCAAAGACATAAAAATATT
TGATTACCTCGTCTCATCTGATGTGGAGCATCGAGATGTTACGGATAAGCAACGAGGGGTCATATATGCTGGCAACC
TTTCTAGGCATAAATGTTCTTTCATATATACTGAAGGATGCGATTTTACTCTCTTTGGTGTCAACTATGAAAATAAA
GATAATCCTAAATATCTTGGAAGTTTTGATGCTCAATCTCCGGAAAAGATTAACCTCCCAGGCATGCAATTTGGACT
CATTTGGGATGGAGATTCTGTCGAAACCTGTAGTGGTGCCTTTGGCGACTATTTAAAGTTTAATAACCCTCATAAGA
CATCTCTTTATCTTTCAATGGAACTTCCAGTATTTATATGGGATAAAGCCGCCCTTGCGGATTTCATTGTAGATAAT
AGAATAGGATATGCAGTGGGATCAATCAAAGAAATGCAAGAGATTGTTGACTCCATGACAATAGAAACTTATAAGCA
AATTAGTGAGAATACAAAAATTATTTCTCAGAAAATTCGAACAGGAAGTTACTTCAGGGATGTTCTTGAAGAGGTGA
TCGATGATCTTAAAACTCGCTAAACGATATGGTCTCTGTGGTTTTATTCGGCTTGTTAGAGATGTCTTATTGACTCG
TGTATTTTACCGGAACTGTAGAATTATTCGATTTCCCTGCTATATTCGCAATGATGGTAGCATTAATTTTGGTGAAA
ATTTCACAAGTGGAGTCGGTCTCAGGCTGGATGCATTTGGACGTGGCGTGATTTTTTTTTCCGATAATGTGCAAGTT
AACGACTATGTTCATATCGCCTCAATTGAGAGCGTTACGATAGGTCGGGATACGCTTATTGCAAGTAAAGTATTTAT
TACCGATCATAATCACGGTTCCTTTAAGCACTCTGATCCAATGAGTTCGCCAAATATACCTCCAGACATGCGCACGT
TGGAATCTTCAGCTGTTGTAATTGGCCAGAGGGTTTGGTTGGGTGAGAATGTGACGGTTTTGCCTGGAACAATTATT
GGTAATGGAGTCGTAGTCGGCGCCAATTCTGTTGTTAGAGGTTCTATTCCCGAAAATACTGTCATTGCGGGAGTACC
AGCAAAAATCATAAAGAAATACAATCATGAGACCAAATTATGGGAAAAAGCATAGTCGTTGTTTCTGCGGTCAATTT
TACCACTGGCGGTCCATTTACCATTTTGAAAAAATTTTTGGCAGCAACTAATAATAAAGAAAATGTCAGTTTTATCG
CATTAGTCCATTCTGCTAAAGAGTTAAAAGAAAGTTATCCATGGGTTAAATTCATTGAGTTTCCTGAGGTTAAAGGG
TCGTGGCTAAAACGTTTGCACTTTGAATATGTAGTTTGTAAAAAACTTTCAAAAGAGCTGAATGCTACGCATTGGAT
TTGTCTGCATGATATTACGGCCAATGTCGTCACTAAAAAAAGATATGTGTATTGTCATAACCCTGCCCCTTTTTATA
AAGGAATTTTATTCCGTGAAATTCTTATGGAGCCTAGCTTTTTCTTATTTAAAATGCTATACGGGCTGATATATAAA
ATAAACATTAAAAAAAATACTGCAGTGTTTGTTCAACAATTCTGGATGAAAGAAAAATTTATCAAGAAATATTCTAT
AAATAACATCATTGTCAGTCGGCCAGAAATTAAATTATCTGATAAAAGCCAACTTACTGATGATGATTCTCAATTTA
AGAATAACCCTTCTGAGTTGACAATATTTTACCCTGCTGTTCCACGAGTATTTAAAAATTACGAGCTTATTATTAGT
GCAGCAAGGAAATTGAAAGAACAATCCAATATTAAATTTCTGCTTACTATCAGTGGTACAGAAAATGCGTATGCAAA
ATATATTATCAGTCTTGCAGAAGGACTGGATAATGTTCATTTCCTCGGGTACTTGGATAAAGAAAAAATCGATCATT
GTTATAATATTTCAGATATAGTTTGTTTTCCCTCTAGGTTAGAAACATGGGGATTGCCGTTGTCTGAGGCTAAAGAG
CGAGGTAAGTGGGTATTAGCATCAGATTTCCCATTTACTAGAGAAACTCTTGGTAGTTATGAAAAGAAAGCTTTTTT
TGATTCTAATAACGATGACATGTTAGTTAAACTTATTATTGACTTCAAAAAAGGTAACCTCAAAAAAGATATCTCTG
ATGCAAATTTCATTTATCGTAATGAAAATGTATTAGTTGGGTTTGATGAACTAGTTAATTTTATTACTGAAGAACAT
TGAAATGGTATATATAATAATCGTTTCCCACGGACATGAAGACTACATCAAAAAATTACTCGAAAATCTTAATGCTG
ACGATGAGCACTACAAGATTATCGTACGCGACAACAAAGACTCTCTATTATTGAAACAAATATGCCAGCATTATGCA
GGCCTGGACTATATTAGTGGAGGTGTATACGGCTTTGGTCATAATAATAATATTGCGGTGGCGTATGTAAAGGAAAA
ATATAGACCCGCAGATGATGATTACATTTTGTTTTTGAATCCCGATATCATCATGAAGCATGATGATTTGCTGACAT
ATATTAAATATGTCGAAAGTAAGCGTTATGCTTTTAGTACATTATGCCTGTTCCGAGATGAAGCGAAATCTTTACAT
GATTATTCCGTAAGAAAATTTCCTGTGCTTTCTGATTTTATTGTGTCATTTATGTTAGGGATTAATAAAACAAAAAT
TCCTAAAGAAAGTATCTATTCTGATACGGTTGTTGATTGGTGCGCAGGATCATTTATGCTGGTACGTTTTTCAGATT
TTGTGCGTGTAAATGGCTTCGATCAAGGTTACTTTATGTACTGTGAAGATATTGACCTGTGCTTGAGGCTTAGCCTG
GCTGGTGTCAGACTTCATTATGTTCCCGCTTTTCATGCGATACATTATGCTCATCATGACAATCGAAGTTTTTTTTC
AAAAGCCTTCAGATGGCACTTAAAAAGTACTTTTAGATATTTAGCCAGAAAACGTATTTTATCAAATCGCAACTTTG
ATCGAATTTCATCAGTTTTTCACCCGTAAGAGCTCGGTACCCGGGCCTAGGGTGTAGGCTGGAGCTGCTTCGAAGTT
CCTATACTTTCTAGAGAATAGGAACTTCGGAATAGGAACTAAGGAGGATATTCATATCCGTCGACGGCGGCCGCCCT
GCAGGCATGCAAGCTTGATCCATATGGATCGCTAGCTTAATTAAATAAAGCCGTAAGCATATAAGCATGGATAAGCT
ATTTATACTTTAATAAGTACTTTGTATACTTATTTGCGAACATTCCAGGCCGCGAGCATTCAGCGCGGTGATCACAC
CTGACAGGAGTATGTAATGTCCAAGCAACAGATCGGCGTAGTCGGTATGGCAGTGATGGGACGCAACCTTGCGCTCA
ACATCGAAAGCCGTGGTTATACCGTCTCTATTTTCAACCGTTCCCGTGAGAAGACGGAAGAAGTGATTGCCGAAAAT
CCAGGCAAGAAACTGGTTCCTTACTATACGGTGAAAGAGTTTGTCGAATCTCTGGAAACGCCTCGTCGCATCCTGTT
AATGGTGAAAGCAGGTGCAGGCACGGATGCTGCTATTGATTCCCTCAAACCATATCTCGATAAAGGAGACATCATCA
TTGATGGTGGTAACACCTTCTTCCAGGACACTATTCGTCGTAATCGTGAGCTTTCAGCAGAGGGCTTTAACTTCATC
GGTACGGGTGTTTCTGGCGGTGAAGAGGGGGCGCTGAAAGGTCCTTCTATTATGCCTGGTGGCCAGAAAGAAGCCTA
TGAATTGGTAGCACCGATCCTGACCAAAATCGCCGCCGTAGCTGAAGACGGTGAACCATGCGTTACCTATATTGGTG
CCGATGGCGCAGGTCACTATGTGAAGATGGTTCACAACGGTATTGAATACGGCGATATGCAGCTGATTGCTGAAGCC
TATTCTCTGCTTAAAGGTGGCCTGAACCTCACCAACGAAGAACTGGCGCAGACCTTTACCGAGTGGAATAACGGTGA
ACTGAGCAGTTACCTGATCGACATCACCAAAGATATCTTCACCAAAAAAGATGAAGACGGTAACTACCTGGTTGATG
TGATCCTGGATGAAGCGGCTAACAAAGGTACGGGTAAATGGACCAGCCAGAGCGCGCTGGATCTCGGCGAACCGCTG
TCGCTGATTACCGAGTCTGTGTTTGCACGTTATATCTCTTCTCTGAAAGATCAGCGTGTTGCCGCATCTAAAGTTCT
CTCTGGTCCGCAAGCACAGCCAGCAGGCGACAAGGCTGAGTTCATCGAAAAAGTTCGTCGTGCGCTGTATCTGGGCA
AAATCGTTTCTTACGCCCAGGGCTTCTCTCAGCTGCGTGCTGCGTCTGAAGAGTACAACTGGGATCTGAACTACGGC
GAAATCGCGAAGATTTTCCGTGCTGGCTGCATCATCCGTGCGCAGTTCCTGCAAAAAATCACCGATGCTTATGCCGA
AAATCCACAGATCGCTAACCTGTTGCTGGCTCCGTACTTCAAGCAAATTGCCGATGACTACCAGCAGGCGCTGCGTG
ATGTCGTTGCTTATGCAGTACAGAACGGTATTCCGGTTCCGACCTTCTCCGCAGCGGTTGCCTATTACGACAGCTAC
CGTGCTGCTGTTCTGCCTGCGAACCTGATCCAGGCACAGCGTGACTATTTTGGTGCGCATACTTATAAGCGTATTGA
TAAAGAAGGTGTGTTCCATACCGAATGGCTGGATTAA
(example O18A rfb locus nucleotide sequence-O18A-EPA production strain
BVEC-L-00559)
SEQ ID NO: 17
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGTGAAAATACTTGTTACTGGTGGCGCAGGATTTATTGGTTCAG
CTGTAGTTCGTCACATTATAAATAATACGCAGGATAGTGTTGTTAATGTCGATAAATTAACGTACGCCGGAAACCGG
GAATCACTTGCTGATGTTTCTGATTCTGAACGCTATGTTTTTGAACATGCGGATATTTGCGATGCACCTGCAATGGC
ACGGATTTTTGCTCAGCATCAGCCGGATGCAGTGATGCACCTGGCTGCTGAAAGCCATGTTGACCGTTCAATTACAG
GCCCTGCGGCATTTATTGAAACCAATATTGTTGGTACTTATGTCCTTTTGGAAGCCGCTCGCAATTACTGGTCTGCT
CTTGATAGCGACAAGAAAAATAGCTTCCGTTTTCATCATATTTCTACTGACGAAGTCTATGGTGATTTGCCTCATCC
AGATGAAGTAAATAATACAGAAGAATTACCCTTATTTACTGAGACGACAGCTTACGCGCCAAGCAGCCCTTATTCCG
CATCCAAAGCATCCAGCGATCATTTAGTCCGCGCGTGGAAACGTACATATGGTTTACCGACAATTGTGACTAATTGC
TCGAACAACTATGGTCCTTATCATTTCCCGGAAAAGCTTATTCCACTGGTTATTCTTAATGCACTGGAAGGTAAGGC
ATTACCTATTTATGGCAAAGGAGATCAGATCCGCGACTGGTTGTATGTTGAAGATCATGCGCGTGCGTTATATACCG
TCGTAACCGAAGGTAAAGCGGGTGAAACTTATAACATTGGTGGGCACAACGAAAAGAAAAACATCGATGTAGTGCTC
ACTATTTGTGATTTGCTGGATGAGATTGTACCGAAAGAGAAATCTTATCGTGAGCAAATCACTTATGTTGCTGATCG
TCCGGGACACGATCGCCGCTATGCTATTGATGCTGAGAAGATTGGTCGCGCATTGGGATGGAAACCACAGGAAACGT
TTGAGAGCGGGATTCGTAAAACGGTGGAATGGTACCTGTCCAATACAAAATGGGTTGATAATGTGAAAAGTGGTGCC
TATCAATCGTGGATTGAACAGAACTATGAGGGCCGCCAGTAATGAATATCCTCCTTTTTGGCAAAACAGGGCAGGTA
GGTTGGGAACTACAGCGTGCTCTGGCACCTTTGGGTAATTTGATTGCTTTTGATGTTCACTCTACTGATTATTGCGG
TGATTTTAGTAATCCTGAAGGTGTAGCTGAAACCGTAAGAAGCATTCGGCCGGATATTATTGTCAATGCAGCCGCTC
ACACCGCAGTAGACAAAGCAGAATCAGAACCGGAGTTTGCACAATTAATTAACGCAACAAGTGTCGAAGCGATTGCG
AAAGCAGCAAATGAAGTTGGAGCCTGGGTTATCCATTACTCGACTGATTACGTCTTCCCTGGAAATGGCGATATGCC
ATGGCTGGAGACGGATGCAACCGCACCACTAAATGTTTACGGTGAAACCAAGTTAGCCGGAGAAAAAGCGTTACAGG
AATATTGCGCGAAGCATCTTATTTTCCGGACCAGCTGGGTCTATGCAGGAAAAGGAAATAACTTCGCCAAAACGATG
TTACGTCTGGCAAAAGAGCGTGAAGAATTAGCGGTTATTAACGATCAGTTTGGTGCGCCAACAGGTGCTGAACTGCT
GGCTGATTGTACAGCACATGCCATTCGTGTCGCACTGAATAAACCGGATGTCGCAGGCTTGTACCATTTGGTAGCCA
GTGGTACCACAACCTGGTACGATTATGCTGCGCTGGTTTTTGAAGAGGCGCGCAAAGCAGGCATTCCCCTTGCACTC
AACAAGCTCAACGCAGTACCAACAACAGCCTATCCTACACCAGCTCGTCGTCCACATAACTCTCGCCTTAATACAGA
AAAATTTCAGCAGAACTTTGCGCTTGTCTTGCCTGACTGGCAGGTTGGCGTGAAACGAATGCTCAATGAATTATTTA
CGACTACAGCAATTTAATAGTTTTTGCATCTTGTTCGTGATGGTGGAGCAAGATGAATTAAAAGGAATGATGAAATG
AAAATGCGTAAAGGTATTATTTTAGCGGGTGGTTCTGGTACACGTCTTTATCCTGTGACTATGGCTGTCAGTAAACA
GCTATTACCTATTTATGATAAACCGATGATCTATTACCCGCTCTCTACACTGATGTTGGCGGGTATTCGCGATATTT
TGATTATCAGTACACCTCAGGATACTCCTCGTTTTCAACAATTGCTGGGTGACGGTAGCCAGTGGGGCCTGAATCTT
CAGTACAAAGTGCAACCTAGCCCAGATGGCCTCGCGCAGGCATTTATCATCGGTGAAGAGTTTATTGGTGGTGATGA
TTGTGCTTTGGTTCTTGGTGATAATATCTTTTACGGTCACGATCTGCCGAAGCTAATGGAGGCCGCTGTTAACAAAG
AAAGTGGTGCAACGGTATTTGCCTATCACGTTAATGATCCAGAACGCTATGGTGTCGTTGAGTTTGATAAAAACGGT
ACGGCAATCAGTCTGGAAGAAAAACCGTTAGAACCAAAGAGTAATTACGCCGTTACAGGTCTGTACTTTTATGATAA
CGACGTGGTTCAGATGGCGAAAAACTTGAAGCCGTCTGCACGTGGTGAGTTAGAAATTACAGATATTAACCGTATTT
ATCTTGAGCAGGGACGTCTGTCTGTCGCGATGATGGGGCGTGGCTACGCGTGGCTGGACACGGGGACTCATCAGAGT
CTGATAGAAGCAAGTAATTTTATTGCGACAATTGAAGAGCGCCAGGGATTGAAGGTTTCCTGTCCTGAAGAGATTGC
ATTTCGTAAAGGTTTTATTGATGTTGAGCAAGTAAGAAAATTAGCTGTACCACTAATAAAGAATAATTATGGGCAGT
ATCTTTATAAAATGACGAAGGATTCAAATTAATGAATGTGATTAGAACTGAAATTGAAGATGTGCTAATTCTGGAGC
CAAGAGTATTTGGTGATGATAGAGGTTTCTTTTATGAGAGCTTTAATCAATCAGCATTTGAACATATTCTAGGCTAT
CCGGTCAGCTTTGTTCAAGACAATCACTCACGTTCATCAAAAAATGTACTCAGAGGCCTTCACTTTCAACGCGGCGA
GTACGCACAAGATAAACTTGTACGCTGCACTCATGGAGCAGTTTTTGATGTTGCTGTTGATATTCGACCCAATTCGG
TATCCTTTGGTAAATGGGTTGGTGTTCTGCTTTCAGCTGATAATAAGCAGCAGTTGTGGATACCAAAAGGGTTTGCT
CATGGCTTTTTGGTTCTGTCTGATATCGCTGAATTTCAATATAAAACTACAAACTATTATCATCCTGAAAGCGATTG
TGGAATATGTTGGAATGATGAACGCATTGCAATTGATTGGCCCCAAACATCAGGGTTAATCCTTTCGCCAAAAGATG
AAAGGCTCTTTACGTTAGATGAGCTTATCAGATTAAAATTAATTGCATGAGGCCGGCCTTAAGGAGGACTAGTCCCG
GCGCGCCATGAGTTTAATCAAAAACAGTTTTTGGAACCTTTGCGGGTATGTACTTCCAGCTATTGTGACACTACCAG
CTTTGGGTATTATGGGGCGAAAATTAGGCCCAGAATTATTTGGTGTATTCACTTTGGCATTAGCTGTTGTGGGTTAT
GCAAGCATTTTTGATGCAGGCCTTACTCGCGCAGTGATACGAGAAGTCGCAATTGAAAAAGATAATGAAGAAAATAA
GTTGAAAATTATTTCTTCAGCGACAGTTGTAATTATTTATTTGAGTTTGGCCGCCTCACTCTTATTATTTTTTTTTA
GTGGTCATATCGCATTGCTACTGAACATTAGTGAGACTTTTTTTCATAATGTAAGTGTCTCGCTTAAAATTCTCGCA
GCATCCATACCATTATTTTTGATTACTCAAATATGGTTGTCAATTTTAGAAGGTGAAGAAAGATTTGGTTTACTTAA
TATCTACAAATCAATTACGGGAGTGATATTAGCAATCTCACCGGCATTATTTATACTTATTAAACCCTCTTTGATGT
ATGCGATAATAGGCTTAGTTCTAGCAAGGTTTTTATGTTTTATTTTGGCTTTTATAATTTGTCACGATAAAGTGCTT
AAAGCTAAACTAACAATCGATATACCAACAATTAAAAGATTGTTTATGTTCGGTGGTTGGATTACAGTAAGTAATAT
CATCAGCCCTGTGCTATCATATTTTGATAGGTTTATTGTTTCAAATCAACTTGGGGCTGCTAATGTTGCTTTTTATA
CTGCACCATCAGAAATTATTTCTCGGCTTAGTATAATTCCAGGTGCGTTTTCAAGAGCCTTATTTCCAAGATTAGCT
AATGCAAATAATTCCGCTGAAAGATATAAAACGAAAAGATTAATTACAATTTCACTTTTAATAATCATCACCCCTAT
TTTTTGTATTGGCGTGTTATTTTCAGAGAAGATAATGGTTTTATGGATGGGGGCATCATTTTTTGGTGAGCCTGGTT
TGGTATTATCAATATTACTGATTGGCTTTATTTTTAATGGATTGGCACAAGTACCATTTGCCAGTATTCAATCCCGA
GGTCATGCTAAGATAACTGCATTTGTTCATCTCTTAGAGTTGTTTCCTTATTTATTACTTTTATTTTACCTCATAAA
AGCACATGGGGTTGTTGGCGCGGGTATTGCGTGGTCAGTGAGGATGATAGTAGATTATATAGCATTAAGTCTTTTGG
ACGGTAAGTATATTAATAAATAAAATTCAAAATGCAAGTTAATAACTCATGGCTTTATTTGGGTAGGTGACAATTTA
TAATGATATATATATTAACTTTAACTCTTCTTCTAGTTATAGCCATAATGTTTTCTCTTCTCGGCACAAAAAGTAGG
ATCACATCTCCATTACCTTTGCATTTTTTACCATGGTTACTAACTTTAATTGTCGGGATAAGTAATTACGATCAATT
TTACGAGTTTAATGAAAGAAGCTTTTACTCTTTGTTGATTTGGTTTACAGTTATTTTTATATTTTATTTCATAGGGG
AACTGGTTAATTATAAACGTGAAAATATAAATGTTTATTATGGTCTTTCACATATTAAATATGAATGTAAAAAATAT
TGGATCATTGTCATCCCAATTTCATTATATACCATTTTCGAAATATATATGGTTGGTATGGGGGGAGCAGATGGATT
CTTTCTCAATTTACGTCTTGCAAATACATTGGAGGGCTATACGGGTAAAAAATTTATCTTAATGCCTGCTGTATATC
CTCTAATGATGGCTATGTTCGCAATTGTTTGTCTAACAAAAACTTCCAAATTAAATAAATACTCCATTTATTTCTGG
ATGTTTTTGTATTGTATTGGCACAATGGGAAAATTTTCAATATTAACGCCAATATTGACATATTTAATTATTTATGA
CTTCAAACATAGATTAAAAGTAAAAAAAACAATAAAGTTTACATTGTTGATAATTATATTAGCTTTAACTTTGCATT
TTACACGTATGGCTGAGAATGACCACTCAACATTTTTATCTATTTTAGGGCTCTATATTTATTCACCAATAATTGCT
TTAGGCCAGTTGAATGAAGTAAATAGTAGTCATTTTGGTGAGTATACGTTTAGATTCATATATGCTATAACTAATAA
AATTGGCCTTATTAAAGAATTGCCAGTAAATACTATTCTTGACTATTCATACGTTCCTGTACCAACAAATGTATATA
CTGCACTTCAACCATTTTACCAGGATTTTGGTTATACTGGCATCATATTTGGAGCAGTATTATACGGACTAATATAT
GTGAGTTTATACACGGCCGGTGTTCGTGGAAATAATACACAGGCATTACTGATTTACGCATTGTTTTCAGTTAGCAG
TGCAACGGCTTTCTTCGCTGAAACGCTAGTAACGAATTTAGCTGGAAATGTGATGTTAGTATTATGTACCATCTTAC
TATGGCGATTTACAGTAATATGCAAACCAGTACAGTAACCATTCTAATGGCCACCTACAATGGCGAGGCCTTCATCA
AAAATCAGATTTTGTCACTACAACAACAAACATTTTCTAACTGGCGGTTATTTATTCAGGATGATGGGTCTACAGAC
AATACTATATCTATAATAAAAAACTTCCAAAAATCTGACTCCAGAATTCGGCTAGTTGATGATAATTTGAAAGGTCA
AGGTGCAGGAAAAAATTTTTTATCGCTGATAAAGTACAGCGAGACAGATTATACAATTTATTGTGACCAAGATGATA
TTTGGTTAGAAAACAAAATATTTGAATTAGTAAAGTATGCAAATGAAATTAAATTGAATGTATCAGATGCGCCTTCG
CTAGTTTATGCTGATGGCTATGCTTATATGGATGGTGAGGGTACAATCGATTTTTCTGGGATATCTAACAATCATGC
TGATCAATTAAAGGATTTTCTTTTTTTTAATGGTGGATACCAAGGATGTTCTATTATGTTCAATCGTGCAATGACCA
AATTTCTTCTGAATTATCGAGGATTTGTATATCTACATGACGATATCACAACATTAGCTGCATACGCTCTTGGTAAA
GTTTATTTTCTCCCGAAATACCTTATGTTATATAGACAGCACACGAATGCGGTAACTGGTATCAAAACATTCCGCAA
TGGATTGACTTCTAAATTTAAATCACCAGTAAACTATCTTTTATCACGAAAACATTATCAGGTAAAAAAATCTTTTT
TTGAATGTAACAGCTCTATCTTATCAGAGACGAATAAAAAAGTTTTTTTGGATTTTATTTCATTTTGTGAATCAAAT
AATAAATTTACAGATTTTTTTAAGTTATGGCGAGGTGGGTTTAGATTAAATAACAGTAGAACTAAATTATTATTAAA
ATTCTTAATACGGAGAAAATTTAGCGAATGATTTCAATACTTACACCTACTTTTAATCGGCAACATACTTTATCAAG
GCTATTCAATTCTCTTATATTACAAACTGATAAAGATTTTGAGTGGATAATAATTGATGATGGTAGTATAGATGCAA
CAGCGGTACTTGTAGAAGATTTTAGAAAAAAATGTGATTTTGACTTGATTTATTGCTATCAGGAAAATAATGGTAAG
CCCATGGCTTTAAACGCTGGTGTTAAAGCTTGTAGAGGCGATTATATCTTTATTGTTGACAGTGATGATGCACTAAC
TCCCGATGCCATAAAATTAATTAAAGAATCAATACATGATTGCTTATCTGAGAAGGAAAGTTTCAGCGGAGTCGGTT
TTAGAAAAGCATATATAAAAGGGGGGATTATTGGTAATGATTTAAATAATTCTTCAGAACATATATACTATTTAAAT
GCGACTGAGATTAGCAATTTAATAAATGGTGATGTTGCATATTGTTTTAAAAAAGAAAGTTTGGTAAAAAATCCATT
CCCCCGTATAGAAGATGAAAAATTTGTTCCAGAATTATATATTTGGAATAAAATAACTGACAAGGCGAAGATTCGAT
TTAACATAAGCAAAGTTATATATCTTTGTGAGTATCTTGATGATGGTCTTTCTAAAAATTTCCATAACCAGCTTAAA
AAATACCCAAAGGGGTTTAAGATTTATTACAAAGATCAAAGAAAACGAGAGAAAACTTATATAAAAAAAACAAAGAT
GCTAATTAGATATTTGCAATGTTGTTATTATGAGAAAATAAAATGAAAATACTATTTGTCATTACAGGTTTAGGCCT
TGGAGGTGCTGAGAAGCAGGTTTGTCTTTTAGCTGATAAATTAAGTTTAAGCGGGCACCATGTAAAGATTATTTCAC
TTGGACATATGTCTAATAATAAAGTCTTTCCTAGCGAAAATAATGTTAATGTCATTAATGTAAATATGTCAAAAAAC
ATTTCTGGAGTTATAAAAGGTTGTGTCAGAATTAGAGATGTTATAGCTAATTTCAAACCAGACATTGTACACAGTCA
TATGTTTCATGCAAACATTATCACTAGATTGTCTGTAATTGGAATCAAAAACAGACCTGGTATTATATCAACTGCAC
ATAATAAAAATGAAGGTGGGTATTTCAGAATGCTCACATATAGAATAACCGATTGTTTAAGTGATTGTTGTACAAAT
GTTAGCAAAGAAGCAGTGGATGAGTTTTTACGGATAAAAGCCTTTAATCCCGCTAAAGCAATTACTATGTATAATGG
GATAGATACCAATAAATTTAAATTTGATTTATTGGCAAGGAGGGAAATTCGAGACGGTATTAATATAAAAAATGATG
ATATATTATTACTTGCTGCAGGTCGTTTAACGTTAGCTAAAGATTATCCTAATTTATTGAATGCAATGACTCTGCTT
CCTGAACACTTTAAACTTATTATTATTGGTGATGGTGAATTGCGTGACGAAATTAATATGCTTATAAAAAAATTGCA
ATTATCTAATAGGGTGTCCTTGTTGGGAGTTAAAAAAAATATTGCTCCCTATTTTTCTGCATGTGATATTTTTGTTC
TCTCTTCTCGTTGGGAAGGATTTGGATTAGTCGTGGCAGAAGCTATGTCATGTGAGCGAATTGTTGTTGGCACGGAT
TCAGGGGGAGTAAGAGAAGTTATTGGTGACGATGATTTTCTTGTACCCATATCTGATTCAACACAACTTGCAAGCAA
AATTGAAAAATTGTCTTTGAGCCAGATACGTGATCACATTGGTTTTCGGAATCGTGAGCGTATTTTAAAAAATTTCT
CAATAGATACTATTATTATGCAGTGGCAAGAACTCTATGGAACTATAATTTGCTCAAAACATGAAAGGTAGATTTAT
ATTTGGAACGTGTCTTTTGTTTGAATTTAATTCAATCTCAATTGAGATTTTTGTATTTCAAAAATACCATCATAGCT
AACGATGATTGGTATTTATTTTAAGATGCTTTCTATAAATATATTGACGTTTTTAATGCGCCGAAACGATTGGGCTG
GGAACAGAGAAGTAAAACTGTTTTGAGAATGAAGAGTTTTTGAGATGTTTATGGATATTAAAAATTGATCCAGTGAA
TTAATTATTTATAATAAATCAAGATTTAATGTTAATAAATGATAATCTTTTCTGACACTCATATTAATTATGAGTGG
TACGTTTGGTAAACGGTAAACTATTATATGACAGCTAGAACAACTAAAGTTTTGCACTTACAATTACTCCCACTCTT
AAGTGGCGTTCAAAGGGTAACATTAAACGAAATTAGTGCGTTATATACTGATTATGATTATACACTAGTTTGCTCAA
AAAAAGGTCCACTAACAAAAGCATTGCTGGAATATGATGTCGATTGTCATTGTATCCCCGAACTTACGAGAGAAATT
ACCGTAAAGAATGATTTTAAAGCATTGTTCAAGCTTTATAAGTTCATAAAAAAAGAAAAATTTGACATTGTGCATAC
ACATTCTTCAAAAACAGGTATTTTGGGGCGAGTTGCTGCCAAATTAGCACGTGTTGGAAAGGTGATCCACACTGTAC
ATGGTTTTTCTTTTCCAGCCGCATCTAGTAAAAAAAGTTATTACCTTTATTTTTTCATGGAATGGATAGCAAAGTTC
TTTACGGATAAGTTAATCGTCTTGAATGTAGATGATGAATATATAGCAATAAACAAATTAAAATTCAAGCGGGATAA
AGTTTTTTTAATTCCTAATGGAGTAGACACTGATAAGTTTTCTCCTTTAGAAAATAAAATTTATAGTAGCACCTTGA
ATCTAGTAATGGTTGGTAGATTATCCAAGCAAAAAGATCCTGAGACATTATTGCTTGCTGTTGAAAAACTGCTGAAT
GAAAATGTTAATGTTAAGCTGACACTTGTAGGAGATGGTGAACTAAAAGAACAGTTAGAAAGCAGGTTCAAACGGCA
AGATGGACGTATAATTTTTCATGGATGGTCAGATAACATTGTTAATATTTTAAAAGTTAATGATCTTTTTATATTAC
CTTCTCTTTGGGAGGGTATGCCATTAGCAATTTTAGAAGCATTGAGCTGTGGACTTCCATGTATAGTCACTAATATT
CCAGGTAATAATAGCTTAATAGAAGATGGCTATAATGGTTGTTTGTTTGAAATTAGAGATTGTCAGTTATTATCTCA
AAAAATCATGTCATATGTTGGTAAGCCAGAACTGATTGCACAGCAATCTACCAATGCACGATCATTTATTCTGAAAA
ATTATGGATTAGTTAAAAGAAATAATAAGGTCAGACAGCTATATGATAATTAAGAGCTCGGTACCCGGGCCTAGGGT
GTAGGCTGGAGCTGCTTCGAAGTTCCTATACTTTCTAGAGAATAGGAACTTCGGAATAGGAACTAAGGAGGATATTC
ATATCCGTCGACGGCGGCCGCCCTGCAGGCATGCAAGCTTGATCCATATGGATCGCTAGCTTAATTAAATAAAGCCG
TAAGCATATAAGCATGGATAAGCTATTTATACTTTAATAAGTACTTTGTATACTTATTTGCGAACATTCCAGGCCGC
GAGCATTCAGCGCGGTGATCACACCTGACAGGAGTATGTAATGTCCAAGCAACAGATCGGCGTAGTCGGTATGGCAG
TGATGGGACGCAACCTTGCGCTCAACATCGAAAGCCGTGGTTATACCGTCTCTATTTTCAACCGTTCCCGTGAGAAG
ACGGAAGAAGTGATTGCCGAAAATCCAGGCAAGAAACTGGTTCCTTACTATACGGTGAAAGAGTTTGTCGAATCTCT
GGAAACGCCTCGTCGCATCCTGTTAATGGTGAAAGCAGGTGCAGGCACGGATGCTGCTATTGATTCCCTCAAACCAT
ATCTCGATAAAGGAGACATCATCATTGATGGTGGTAACACCTTCTTCCAGGACACTATTCGTCGTAATCGTGAGCTT
TCAGCAGAGGGCTTTAACTTCATCGGTACCGGTGTTTCTGGCGGTGAAGAGGGGGCGCTGAAAGGTCCTTCTATTAT
GCCTGGTGGCCAGAAAGAAGCCTATGAATTGGTAGCACCGATCCTGACCAAAATCGCCGCCGTAGCTGAAGACGGTG
AACCATGCGTTACCTATATTGGTGCCGATGGCGCAGGTCACTATGTGAAGATGGTTCACAACGGTATTGAATACGGC
GATATGCAGCTGATTGCTGAAGCCTATTCTCTGCTTAAAGGTGGCCTGAACCTCACCAACGAAGAACTGGCGCAGAC
CTTTACCGAGTGGAATAACGGTGAACTGAGCAGTTACCTGATCGACATCACCAAAGATATCTTCACCAAAAAAGATG
AAGACGGTAACTACCTGGTTGATGTGATCCTGGATGAAGCGGCTAACAAAGGTACGGGTAAATGGACCAGCCAGAGC
GCGCTGGATCTCGGCGAACCGCTGTCGCTGATTACCGAGTCTGTGTTTGCACGTTATATCTCTTCTCTGAAAGATCA
GCGTGTTGCCGCATCTAAAGTTCTCTCTGGTCCGCAAGCACAGCCAGCAGGCGACAAGGCTGAGTTCATCGAAAAAG
TTCGTCGTGCGCTGTATCTGGGCAAAATCGTTTCTTACGCCCAGGGCTTCTCTCAGCTGCGTGCTGCGTCTGAAGAG
TACAACTGGGATCTGAACTACGGCGAAATCGCGAAGATTTTCCGTGCTGGCTGCATCATCCGTGCGCAGTTCCTGCA
AAAAATCACCGATGCTTATGCCGAAAATCCACAGATCGCTAACCTGTTGCTGGCTCCGTACTTCAAGCAAATTGCCG
ATGACTACCAGCAGGCGCTGCGTGATGTCGTTGCTTATGCAGTACAGAACGGTATTCCGGTTCCGACCTTCTCCGCA
GCGGTTGCCTATTACGACAGCTACCGTGCTGCTGTTCTGCCTGCGAACCTGATCCAGGCACAGCGTGACTATTTTGG
TGCGCATACTTATAAGCGTATTGATAAAGAAGGTGTGTTCCATACCGAATGGCTGGATTAA
(example O25B rfb locus nucleotide sequence-O25B-EPA production strain
stGVXN4459)
SEQ ID NO: 18
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGCTAGCAGTGAAGATACTTGTTACTGGTGGCGCAGGATTTATT
GGTTCTGCTGTTGTTCGTCACATAATAAATAATACGCAAGATAGTGTTGTTAATGTCGATAAATTAACATACGCCGG
AAACCTGGAATCACTTGCAGATGTTTCTGATTCTGAACGCTATTTCTTTGAACATGCGGATATTTGTGATGCAGCTG
CAATGGCACGGATTTTTGCTCAGCATCAGCCGGATGCAGTGATGCACCTGGCAGCTGAAAGCCATGTTGACCGTTCA
ATTACAGGCCCTGCGGCATTTATTGAAACCAATATTGTGGGTACTTATGTCCTTTTAGAAGCGGCTCGGAATTATTG
GTCTGGTCTGGATGATGAAAAGAAAAAAAACTTCCGTTTTCATCATATTTCTACTGATGAGGTGTATGGTGACTTAC
CCCATCCGGATGAAGTAAATAGCAATGAAACGTTGCCGCTATTTACGGAAACGACAGCATACGCGCCAAGTAGTCCA
TATTCTGCTTCTAAAGCTTCCAGCGATCATTTGGTTCGCGCATGGAAACGTACTTATGGTTTACCGACCATTGTGAC
TAATTGCTCGAACAACTATGGTCCTTATCATTTCCCGGAAAAGCTTATTCCACTGGTTATTCTTAATTCACTGGAAG
GTAAGGCATTACCTATTTATGGCAAAGGAGATCAGATCCGCGACTGGTTGTATGTAGAGGATCATGCTCGAGCGTTA
TATACCGTCGTAACCGAAGGTAAAGCGGGCGAAACTTATAACATTGGTGGACACAACGAAAAGAAAAACATCGACGT
AGTGTTCACTATTTGTGATTTGTTGGATGAGATAGTCCCGAAAGAGAAATCTTACCGCGAGCAAATTACTTATGTTA
CCGATCGTCCGGGACACGATCGCCGTTATGCGATTGATGCTGAGAAGATTGGTCGCGAATTGGGATGGAAACCACAG
GAAACGTTTGAGAGTGGGATTCGTAAAACGGTGGAATGGTACCTGTCCAATACAAAATGGGTTGATAATGTGAAAAG
TGGTGCCTATCAATCGTGGATTGAACAGAACTATGAGGGCCGCCAGTAATGAATATCCTCCTTTTTGGCAAAACAGG
GCAGGTAGGTTGGGAACTACAGCGTGCTCTGGCACCTCTGGGTAATTTGATTGCTCTTGATGTTCACTCCACTGATT
ACTGTGGTGATTTTAGTAATCCTGAAGGTGTAGCTGAAACCGTAAGAAGCATTCGGCCTGATATTATTGTCAACGCA
GCCGCTCACACCGCAGTAGACAAAGCAGAATCAGAACCGAAGTTTGCACAATTACTGAACGCGACGAGTGTCGAAGC
GATCGCGAAAGCAGCCAATGAAGTCGGCGCCTGGGTTATTCACTACTCTACTGACTACGTATTTCCGGGGACCGGTG
AAATACCATGGCAGGAGGAGGATGCAACCGCACCGCTAAATGTTTACGGTGAAACCAAGTTAGCGGGAGAAAAAGCA
TTACAAGAGCATTGTGCGAAGCACCTTATTTTCCGGACCAGCTGGGTCTATGCAGGTAAAGGAAATAACTTCGCCAA
AACAATGTTGCGTCTGGCAAAAGAGCGTGAAGAATTAGCCGTTATTAATGATCAGTTTGGTGCGCCAACTGGCGCAG
AGTTACTGGCTGATTGTACGGCACATGCTATTCGTGTGGCACTGAATAAACCGGAAGTCGCAGGCTTGTACCATCTG
GTAGCTAGTGGTACCACAACGTGGCACGATTATGCTGCGCTGGTTTTTGAAGAGGCGCGCAAAGCAGGCATTCCCCT
TGCACTCAACAAGCTCAACGCAGTACCAACAACAGCCTATCCTACACCAGCTCGTCGTCCACATAACTCTCGCCTTA
ATACAGAAAAATTTCAGCAGAACTTTGCGCTTGTCTTGCCTGACTGGCAGGTTGGCGTGAAACGAATGCTTAACGAA
TTATTTACGACTACAGCAATTTAATAGTTTTTGCATCTTGTTCGTAATGGTGGAGCAAGATGTATTAAAAGGAATGA
TGAAATGAAAACGCGTAAAGGTATTATTTTGGCGGGTGGTTCTGGTACTCGTCTTTATCCTGTGACGATGGCCGTCA
GTAAACAGCTGTTACCGATTTATGATAAACCGATGATCTATTACCCGCTCTCTACACTGATGTTAGCGGGTATTCGC
GATATTCTGATTATCAGTACACCACAGGATACTCCTCGTTTTCAACAACTGCTGGGTGACGGGAGCCAGTGGGGCCT
GAATCTTCAGTACAAAGTGCAACCGAGTCCGGATGGTCTTGCGCAGGCGTTTATTATCGGTGAAGAGTTTATTGGTG
GTGATGATTGTGCTTTGGTACTTGGTGATAATATCTTCTACGGCCACGACCTGCCGAAGTTAATGGACGTAGCTGTT
AACAAAGAAAGTGGTGCAACGGTATTTGCCTATCACGTTAATGATCCTGAACGTTATGGTGTCGTGGAGTTTGATAA
TAACGGTACTGCAATTAGCCTGGAAGAAAAACCGCTGGAACCAAAAAGTAACTATGCGGTTACTGGGCTTTATTTCT
ATGACAATGACGTTGTGGAAATGGCGAAAAACCTTAAGCCTTCTGCCCGAGGTGAACTGGAAATTACCGATATTAAC
CGTATTTATATGGAACAAGGACGTTTGTCTGTCGCTATGATGGGGCGTGGCTATGCATGGCTGGATACAGGGACGCA
TCAAAGTCTTATTGAAGCAAGCAACTTCATTGCCACCATTGAAGAGCGCCAGGGACTAAAGGTTTCCTGTCCGGAAG
AAATTGCTTATCGTAAAGGGTTTATTGATGCTGAGCAGGTAAAAGTATTAGCCGAACCGTTGAAGAAAAATGCTTAT
GGTCAGTATCTGCTCAAAATGATTAAAGGTTATTAATAAGATGAACGTAATTAAAACTGAAATTCCTGATGTGCTGA
TTTTTGAACCAAAAGTTTTTGGGGATGAACGTGGCTTCTTTTTTGAGAGTTTTAATCAGAGGATTTTTGAAGAAGCA
GTAGGTCGTAAGGTTGAGTTTGTTCAGGATAACCATTCTAAGTCCAGTAAAGGTGTTTTACGTGGTCTTCATTATCA
GTTAGAACCTTATGCTCAAGGAAAACTGGTGCGCTGTGTTGTTGGCGAGGTTTTTGATGTTGCGGTTGATATTCGTA
AATCGTCACCTACATTTGGGAAATGGGTTGGGGTGAATTTGTCTGCTGAGAATAAGCGTCAGTTGTGGATTCCTGAG
GGATTTGCACATGGTTTTTTGGTGCTGAGTGATTTAGCAGAAGTTTTATATAAAACGAATCAATATTATGCTCCATC
ACATGAAAAAAATATTATATGGAATGACCTCTTGCTTAATATTAAATGGCCGAGCACAGCACTGATCACTCTGTCTG
ATAAGGATGCAAATGGGGAAAGATTTGAACTAAGTGAGTTTTGAAATGTCTCTCTTAAAACATAGTATATGGAATGT
TGCGGGCTACTTTATACCAACATTAATTGCAATTCCCGCCTTTGGATTAATTGCGAGGAAAATTGGTGTAGAACTAT
TTGGTTTGTATACGTTAGCAATGATTTTTATAGGGTATGCAAGTATATTTGATGCTGGGTTAACAAGAGCTGTTGTG
CGTGAAATAGCATTACTAAAAAACAGAGTGGACGATTGTAATACGATAATAGTAACTTCTATTATCGCTGTGATATT
TTTAGGGTTTATCGGAGGCGGGGGAGTGTTTCTGCTTAAAGGCGATATTATTGAACTGTTAAATATCTCACCAATAT
ATTACGCCGATTCGATAAAGTCTCTAGTATTATTATCATCTCTGATACCTGTATTCTTAGTCACGCAAATACTATTA
GCAGAGCTTGAGGGTCGGGAATATTTTGGGATTCTAAATATACAAAAAAGTGTAGGGAATTCTTTAATTGCAGGGTT
ACCTGCATTATTTGTTTTAATTAATCAAACGCTTTTTTCTGCAATTATTGGTGTAGCGATTGCAAGAGTTATATGCT
TGTGGTTAAGCTACATTATGAGCAGGGAAAGAATAACTATCGATATCTCATTTTTTTCAATAACTGTTTTAAAGCGG
TTATTTAGATATGGCGGGTGGGTAACTATAAGTAACATAATATCTCCTATATTAGCGAGTATGGATAGATTTATTCT
ATCCCATATCCAGGGAGCATCAAAAATATCATTCTATACAGTCCCTAATGAGCTGGTAACTAGGCTTGGAATAGTTC
CAGGCTCTCTTGGGAAAGCTGTTTTTCCAAAATTAAGTCATGCAAGGAATTTTACAGCGTCATATGCAGAGCAAAAA
AAAGCTTATATATTAATGACTGTCATTGTAATGCCTTTGGTTTTATTTGTATATTATTACGCAAAGTTTATTTTAAC
ATTGTGGATGGGGGCTGAGTATGCAGGGATTTCGGTCGAAATATTACGGATTATGCTTATAGGGTATATTTTTAACT
GTTATTCACAAATCTCTTTTGCCAACATACAGGCCTTTGGAAAAGCAAAATACACTGCATACATCCATATGATGGAA
TTTATTCCTTATTTGATAATGTTATATATAATTTCAAAGGAATATGGGGTTATTGGTGTTGCGTGGTTATGGACAAT
TCGAGTAATAATTGATTTTTTGATGCTTTTATATATGAGTTATCGTTGTAATAATCTTATGAAAAAAGGGTAGCCTG
ATGATATATATTGTGGTATTAAATTGGAATGGGGCTATAGATACCATTAATTGTGTTAAAAGTTTAATGGATTTAAA
TGTTAGCGATTATAAAATTATCATTGTTGATAACTGTTCTATGGATAACTCATATGATACTATAAAAGAAAATCTTA
ATTCATTATATATTGCTGATAAAAGTATCATTGAGGTGAAGTATGAGGATAGAAATAAATATAAAACCTTAGAAAAC
GATAAAATCATATTAATACAATCTCCGCAAAATAATGGGTACGCAAGTGGTAATAATATTGGCATAGAGTTCGCTCT
TAATCAGGAGAATATGAAATACGTCTGGGTTCTGAATAATGATACTGAAGTGGATAAAGAGGCTTTAACTCATTTAA
TTAGTAAATGTGATTCAGATAAAAGTATAGGGATTTGCGGTTCTCGTTTAGTCTATTTTGCCGACAGAGAGATGCAG
CAAGGACTAGGTGGGGTGCATAACAAATGGTTATGCACTACAAAAAATTATGAAATGGGAAGATTAGTTTCCAAAAA
ATATGATGATGAAGTCATTAGTAATGATATAGATTATATAATTGGCGCATCGATGTTTTTCTCTAGAGAATGTTTGG
AAACAGTTGGATTGATGAATGAAGAATATTTTTTATACTATGAAGAGTTAGATATTTGCCTCAGAGCAAAAGCAAAG
AACTTTAAATTAGGTATTTGCTCAGAAAGTTTGGTTTATCATAAAATAGGTGCAAGTACTGATGGGGGAAAGAGCAT
GATGGCTGATCTTTGCTCAATAAAAAATAGGCTGGTCATTACAGAAAGGTTTTATCCCCAATATTATTGGACGGTAT
GGTTGTCACTTTTTGTTGTAGCATTTAACCGTGCTAGAAGAGGTGAGTTTAATAAGATGAAAAGATGTTTGAATGTT
ATGTTTAACTTCAAACGAAACAAAGGTAGCAAATGCCATTAGAATATGCACTTAATCATGGTGTTAATAAATCTATA
GTTTGATATGTTATTAAAGGGTATTTAATGAAAGTGGCTTTTTTATCTGCTTATGATCCACTATCTACATCCAGTTG
GTCTGGCACACCTTATTATATGCTAAAGGCATTATCGAAGAGAAATATTTCCATTGAAATATTAGGACCGGTAAATA
GCTATATGATATACATGTTAAAAGTATATAAATTAATATTAAGGTGTTTCGGAAAAGAATATGATTATAGTCATTCG
AAGTTGCTTTCCAGGTATTACGGTAGAATATTCGGTAGGAAATTAAAAAAAATTGATGGTTTGGATTTTATTATCGC
ACCTGCAGGTTCCTCACAAATTGCTTTTTTAAAAACAACCATACCAATAATATATCTATCGGATACAACATATGATC
AATTAAAAAGCTATTATCCGAATTTAAATAAAAAAACAATTATAAATGATGAGGATGCAAGTTTAATCGAACGCAAG
GCTATTGAAAAAGCAACAGTAGTATCTTTCCCATCTAAATGGGCAATGGATTTTTGCAGGAATTATTACAGATTAGA
TTTTGATAAATTAGTTGAAATACCATGGGGGGCTAATTTATTTGATGATATTCACTTTGCTAATAAAAATATAATTC
AAAAGAATAGTTATACTTGTCTTTTCTTGGGAGTTGATTGGGAAAGAAAAGGTGGGAAAACAGCCTTGAAAGCAATT
GAATATGTAAGGCAGTTATATGGGATCGATGTTAGACTAAAAATTTGTGGATGTACTCCGAATCAAAAGATTTTACC
TACTTGGGTTGAATTAATTGATAAAGTAGATAAAAATAACGTTGACGAATATCAGAAATTCATCGATGTGTTATCTA
ACGCTGATATACTTCTTTTACCAACCATTGCTGAATGTTATGGAATGGTATTTTGTGAAGCTGCTGCTTTTGGATTG
CCTGTTGTCGCTACAGATACAGGTGGAGTCAGTTCTATAGTTATCAACGAAAGGACGGGGATATTAATTAAAGACCC
GTTAGACTATAAGCACTTTGGAAATGCAATTCATAAAATAATTAGTTCCGTAGAGACTTATCAAAACTACTCCCAAA
ACGCAAGAATTAGATATAATAATATATTGCATTGGGACAATTGGGCTAAAAAGATAATTGAGATTATGTATGAGCAT
AAGAATAGAAGAATCAAATAGCACAAAAAGAATTATATGTTTATTTATACTTTTTCTTGTTTTCCCTGATTTTTTGT
TTTATACATTAGGGGTTGATAATTTTAGCATTTCAACGATAATCTCAATTACATTGCTTTTTGTTTTTTTAAGAGCT
AAAAATATTTGCAAAGATAATTTTCTAATAATAGTAGCGTTATTCATATTGTTGTGTTTTAACTGTTTGTTAAGTAT
GCTATTTAATATTGAACAGGCTTTAACATTTAAAGTTGTACTTTCAATATATAGCATCTTAATAATGGCATACGTCT
CCTCTTGTTATGCACAGACGTTGTGGTTATGTTCTGAAGAAATACTTAAGAGATCCGTCTTTTATTTGTTCGCATTT
CTTTGCCTTATTGGCATTATAAGTATTCTTTTACAGAAGACTGAGATTATACATGATAAAAGTATGATTCTTTTTCC
TGAACCATCAGCATTTGCATTGGTTTTTATACCTATCTTTTCATTTTGTTTATACTATACAAGAGGGGGGGGGCTAC
TATTGCTCTATATATTATCTTTGGGTATTGCGTTAGGTATCCAGAATTTAACAATGTTGGTAGGCATTGTGATTAGT
GTTTTTGTGATGAAAAAAATAACTATAAGGCAAACTATTGTTATACTTTTGGGGGCATGGATTTTTTCCATGATATT
AAGTGATTTAGACATTTCTTACTATACATCGCGGCTTGATTTTAAAAATACTACGAACCTATCAGTGCTTGTATATC
TTTCAGGAATTGAAAGAGCTTTCTTGAATTTTATTACAAGTTATGGTCTTGGTATTGGTTTTCAACAAATGGGAGTG
AATGGGGAGATAGGAATATATCAACAAATTTTAGCTGAACTTGATGCCCCTATGTTAAATATATACGATGGCTCATT
TATTTCTTCTAAGTTAATATCTGAGTTTGGGGTTATTGGTGCATTAATGTGTATTTTCTATTTTTTTTATTTTTCCC
GATTTTATCTGCGTTTCAAAAAAAGTAAGAGATATTCACCGCAGTATATTTTAGCATATAGCTTCTACATGTGTTTC
TTCATCCCTCTTTTTATACGTGGTGCTGGTTATATAAACCCCTATGTGTTTATGTTATTTTCATCAATATTTTTGTG
CAAATATCACGCTAAAAATATCTTGATGAAATCTAATGTCCAGATAGCTATATAATAGTAGATTATATTATCATTAT
CACGTAAATTACATATTAATAGCATATATGATAACTAGGACATAAATAATGTGCATTAAAAAAAAACTTAAGTTAAT
TAAACGATATGGCCTTTATGGTGGTCTTAGGCTTCTTAAAGATATATTCTTAACAAAATTTTTATTTTGTTCAAATG
TTAGGATTATTAGATTTCCATGTTATATTAGAAAAGATGGAAGTGTTAGTTTTGGAAAAGGTTTTACATCAGGTGTA
GGATTACGAGTTGATGCATTTATGGATGCCGTAGTTTCCATTGGAGAAAATGTTCAAATTAATGACTATGTTCACAT
CGCGGCTATTAATAATGTCATTATTGGTAGAGATACATTAATAGCAAGTAAAGTATTTATTAGTGATCATAATCATG
GTATTTTTTCTAAATCCGATATCCATAGTTCACCAACTATTATTCCTTCGTCTAGGCCCCTTGAATCTGCACCTGTG
TATATTGGAGAGCGTGTGTGGATTGGCGAAAATGTGACAATATTACCAGGTGCGTGTATAGGTAATGGTGTAGTTAT
TGGCGCAAACAGTGTTGTTCGTGGTGAGATTCCTAATAATGTGATCATTGCTGGTGTTCCAGCTAAAATTGTTAAAA
AATATAACTATGAGCGTATGCAATGGGAAAGAATATAGTTGTAATATCGGCTGTTAATTTTACAACCGGAGGCCCCT
TTACCGTACTAAAAAATGTGCTTACAGCAACTAAAGATAGAGCCGAATGTAAATTTATTGCACTGGTTCATAGCTCT
GCTGAACTAATGGAATTATTTCCGTGGGTTGAATTTATAGAGTATCCAGAAGTCAAGTCTTCGTGGGTTAAAAGATT
ATATTTCGAATATATAACTTGCAATAGATTATCTAAGGTGATTAAGGCAACTCATTGGGTATGCTTACATGATATTA
CAGCAAATGTTAGTGTACCCTATAGATTTGTTTATTGCCACAATCCTGCACCGTTCTATAAATATTTAAGCTATCGA
GATATTATAGGAGAACCTAAATTTTATCTTTTTTATCTTTTTTATGGGCTTTTATACAATATCAATATAAAAAAGAA
CACAGCAGTTTTTGTTCAGCAGCAGTGGCTAAAAAAAGAATTCGAAAAAAAATATAAGTTAAAGAATGTTGTTGTTA
GTCGCCCTGAAGATATTTGCCCTTTTGAAAGTGATGGTTTGGTAAGAAATAATAATAAAAAGGATGTGAGGATATTT
TACCCAGCAGTGCCCCGTATATTTAAAAACTTTGAAGTTATCATACGTGCTGCACAAATATTACAAGATAAAAATAT
TCATTTTTATCTTACTTTTGATGGTACTGAAAATAAGTATGCAAAAAGAATATATAAATTAGCTTCCGAACTGAAAA
ATGTACATTTCCTCGGTTACCTTAATGCAACCGAGATGGTTAACTTTTATCAAGATTCAGATATTATTTGTTTCCCA
TCGAAACTAGAAACGTGGGGATTACCATTATCAGAAGCTAAAACATACAAAAAATGGATATTTGCGGCAGACTTACC
TTATGCTCATGAAGTTTTATATAACTATTCAAAAACTAGATATTTTCCATTTGACGATGAGAAAATACTTGTTCGCT
ACATATTAGAGTACACAAGTAAAAATATGCATGAAGATATAAAAAATAGTAGGGTGAATTTTAATAATGATGCATTG
ACTGGTTTTGAACAGTTTATTGAATATATCCTCAAGGGGAACTGACGTGGTTTATATTATAATCGTTTCACATGGCC
ATGATGACTATATAGAAAATCTTTTATTAAATTTAAAGTTGCCCTCTGGAAGATTTAAAATAATAGTTCGTGATAAC
AAAAGTTCAATGGTTTTAAAAAAAACATGCGAAAAAAATTGCGTAACCTATTTGCATGGAGGGCAATATGGATTTGG
ACATAATAATAACATAGCAGTGTCATATATAATTAATAACTTCATGATTATGAATAATGATTATTTTCTCTTTCTTA
ACCCCGATGTATTCATAACCAGTGAAAGTTTGATTAATTATGTTGATTATATAATTAGTAATGATTATAAGTTTAGC
ACATTATGTCTTTATCGAGATTTTACTAAAAGCAAACATGATTATTCAATACGGAGTTTTCCAACTTTATATGATTT
TCTTTGTTCTTTTTTATTGGGGGTGAATAAAAGTAAAATTAAGAAGGAAAATATACTTTCTGATACTGTAGTTGATT
GGTGTGCTGGCTCATTTATGCTTATTCATGCTTTAAGTTTCTTAAATGTGAATGGTTTTGATCAAAAATATTTTATG
TATTGTGAAGATATTGACCTTTGTATGCGTTTAAAATTAAGTGGAGTAGATCTTTACTATACTCCCCATTTTGATGC
TATTCATTATGCGCAGCATGAAAATAGAAGAATATTTACTAAAGCATTTCGATGGCATATAAGGAGTATTACGCGCT
ACATATTACGGAAACCAATTCTTTCTTATAAAAACTATAGAAAAATTACATCCGAACTGGTAAAGTGATTAAGGATC
CGTGTAGGCTGGAGCTGCTTCGAAGTTCCTATACTTTCTAGAGAATAGGAACTTCGGAATAGGAACTAAGGAGGATA
TTCATATGGATAAAGCCGTAAGCATATAAGCATGGATAAGCTATTTATACTTTAATAAGTACTTTGTATACTTATTT
GCGAACATTCCAGGCCGCGAGCATTCAGCGCGGTGATCACACCTGACAGGAGTATGTAATGTCCAAGCAACAGATCG
GCGTAGTCGGTATGGCAGTGATGGGACGCAACCTTGCGCTCAACATCGAAAGCCGTGGTTATACCGTCTCTATTTTC
AACCGTTCCCGTGAGAAGACGGAAGAAGTGATTGCCGAAAATCCAGGCAAGAAACTGGTTCCTTACTATACGGTGAA
AGAGTTTGTCGAATCTCTGGAAACGCCTCGTCGCATCCTGTTAATGGTGAAAGCAGGTGCAGGCACGGATGCTGCTA
TTGATTCCCTCAAACCATATCTCGATAAAGGAGACATCATCATTGATGGTGGTAACACCTTCTTCCAGGACACTATT
CGTCGTAATCGTGAGCTTTCAGCAGAGGGCTTTAACTTCATCGGTACCGGTGTTTCTGGCGGTGAAGAGGGGGCGCT
GAAAGGTCCTTCTATTATGCCTGGTGGCCAGAAAGAAGCCTATGAATTGGTAGCACCGATCCTGACCAAAATCGCCG
CCGTAGCTGAAGACGGTGAACCATGCGTTACCTATATTGGTGCCGATGGCGCAGGTCACTATGTGAAGATGGTTCAC
AACGGTATTGAATACGGCGATATGCAGCTGATTGCTGAAGCCTATTCTCTGCTTAAAGGTGGCCTGAACCTCACCAA
CGAAGAACTGGCGCAGACCTTTACCGAGTGGAATAACGGTGAACTGAGCAGTTACCTGATCGACATCACCAAAGATA
TCTTCACCAAAAAAGATGAAGACGGTAACTACCTGGTTGATGTGATCCTGGATGAAGCGGCTAACAAAGGTACCGGT
AAATGGACCAGCCAGAGCGCGCTGGATCTCGGCGAACCGCTGTCGCTGATTACCGAGTCTGTGTTTGCACGTTATAT
CTCTTCTCTGAAAGATCAGCGTGTTGCCGCATCTAAAGTTCTCTCTGGTCCGCAAGCACAGCCAGCAGGCGACAAGG
CTGAGTTCATCGAAAAAGTTCGTCGTGCGCTGTATCTGGGCAAAATCGTTTCTTACGCCCAGGGCTTCTCTCAGCTG
CGTGCTGCGTCTGAAGAGTACAACTGGGATCTGAACTACGGCGAAATCGCGAAGATTTTCCGTGCTGGCTGCATCAT
CCGTGCGCAGTTCCTGCAGAAAATCACCGATGCTTATGCCGAAAATCCACAGATCGCTAACCTGTTGCTGGCTCCGT
ACTTCAAGCAAATTGCCGATGACTACCAGCAGGCGCTGCGTGATGTCGTTGCTTATGCAGTACAGAACGGTATTCCG
GTTCCGACCTTCTCCGCAGCGGTTGCCTATTACGACAGCTACCGTGCTGCTGTTCTGCCTGCGAACCTGATCCAGGC
ACAGCGTGACTATTTTGGTGCGCATACTTATAAGCGTATTGATAAAGAAGGTGTGTTCCATACCGAATGGCTGGATT
AA
(example O75 rfb locus nucleotide sequence-O75-EPA production strain
stLMTB11737)
SEQ ID NO: 19
ATGACGAATTTAAAAGCAGTTATTCCTGTAGCGGGTCTCGGGATGCATATGTTGCCTGCCACTAAGGCGATACCCAA
AGAGATGCTACCAATCGTCGACAAGCCAATGATTCAGTACATTGTTGACGAGATTGTGGCTGCAGGGATCAAAGAAA
TCCTCCTGGTAACTCACGCGTCCAAGAACGCGGTCGAAAACCACTTCGACACCTCTTATGAGTTAGAATCACTCCTT
GAGCAGCGCGTGAAGCGTCAACTGCTGGCGGAAGTACAGTCCATCTGTCCGCCGGGCGTGACCATTATGAACGTGCG
TCAGGGCGAACCTTTAGGTTTAGGCCACTCCATTTTGTGTGCGCGACCTGCCATTGGTGACAACCCATTTGTCGTGG
TACTGCCAGACGTTGTGATCGACGATGCCAGCGCCGACCCGCTACGTTACAACCTTGCTGCCATGATTGCACGTTTC
AACGAAACGGGCCGCAGCCAGGTGCTGGCAAAACGTATGCCGGGTGACCTCTCTGAATACTCCGTCATCCAGACTAA
AGAGCCGCTGGACCGTGAGGGTAAAGTCAGCCGCATTGTTGAATTTATCGAAAAACCGGATCAGCCGCAGACGCTGG
ACTCAGACATCATGGCCGTAGGTCGCTATGTGCTTTCTGCCGATATTTGGCCGGAACTGGAACGTACTCAGCCTGGT
GCATGGGGACGTATTCAGCTGACTGATGCTATTGCCGAGCTGGCGAAAAAACAATCCGTTGATGCAATGCTGATGAC
CGGCGACAGTTACGACTGCGGCAAAAAAATGGGCTATATGCAGGCGTTTGTGAAGTATGGCCTACGCAACCTGAAAG
AAGGGGCGAAGTTCCGTAAAGGTATTGAGAAGCTGTTAAGCGAATAATGAAAATCTGACCGGATGTAACGGTTGATA
AGAAAATTATAACGGCAGTGAAAATTCGCAGCAAAAGTAATTTGTTGCGAATCTTCCTGCCGTTGTTTTATATAAAC
CATCAGAATAACAACGAGTTAGCAGTAGGGTTTTATTCAAAGTTTTCCAGGATTTTCCTTGTTTCCAGAGCGGATTG
GTAAGACAATTAGCGTTTGAATTTTTCGGGTTTAGCGCGAGTGGGTAACGCTCGTCACATCATAGGCATGCATGCAG
TGCTCTGGTAGCTGTAAAGCCAGGGGCGGTAGCGTGCATTAATACCTCTATTAATCAAACTGAGAGCCGCTTATTTC
ACAGCATGCTCTGAAGTAATATGGAATAAATTAAGCTAGCAGTGAAGATACTTGTTACTGGTGGCGCAGGATTTATT
GGTTCTGCTGTTGTTCGTCACATAATAAATAATACGCAAGATAGTGTTGTTAATGTCGATAAATTAACATACGCCGG
AAACCTGGAATCGCTCGCTGAAATTTCTGATTCTGAACGTTATTCATTTGAGCATGCAGATATCTGCGATGCCGAAG
CGATGGCTCGTATTTTCGCACAGCACCAGCCAGACGCGGTGATGCACCTGGCAGCAGAGAGCCACGTTGACCGCTCA
ATAACTGGCCCTGCGGCATTTATTGAAACCAATATTGTGGGTACTTATGTTCTTTTAGAAGCGGCGCGCAATTATTG
GTCTGGTCTGGATGATGAAAAGAAAAAAAACTTCCGCTTTCATCATATTTCTACTGATGAGGTGTATGGTGACTTAC
CCCATCCGGATGAAGTAAATAGCAATGAAACGTTGCCGCTATTTACGGAAATGACAGCATACGCGCCAAGTAGTCCA
TATTCTGCTTCTAAAGCTTCCAGCGATCATTTGGTTCGCGCATGGAAACGTACTTATGGTTTACCGACCATTGTGAC
TAATTGCTCGAACAACTATGGTCCTTATCATTTCCCGGAAAAGCTTATTCCACTGGTTATTCTTAATGCACTGGAAG
GTAAGGCATTACCTATTTATGGCAAAGGAGATCAGATCCGCGACTGGTTGTATGTAGAGGATCATGCTCGAGCGTTA
TATACCGTCGTAACCGAAGGTAAAGCGGGCGAAACTTATAACATTGGTGGACACAACGAAAAGAAAAACATCGACGT
AGTGTTCACTATTTGTGATTTGTTGGATGAGATAGTCCCGAAAGAGAAATCTTATCGTGAGCAAATTACCTATGTTG
CTGATCGCCCAGGGCATGATCGCCGTTATGCAATTGATGCCGATAAAATTAGCCGCGAATTGGGCTGGAAACCACAG
GAAACGTTTGAGAGCGGGATTCGTAAAACTGTGGAATGGTATCTGTCCAATACAAAATGGGTTGATAATGTGAAAAG
TGGTGCCTATCAATCGTGGATTGAACAGAACTATGGGGGCCGCCACTAATGAATATCCTCCTTTTTGGCAAAACAGG
GCAGGTTGGTTGGGAACTACAGCGTGCTCTGGCACCTCTGGGTAATTTGATTGCTCTTGATGTTCACTCCACTGATT
ACTGTGGTGATTTTAGTAACCCTGAAGGTGTGGCTGAAACCGTTAGAAGCATTCGGCCTGATATTATTGTCAACGCA
GCCGCTCACACCGCAGTAGACAAAGCAGAATCAGAACCGGAGTTTGCACAATTACTGAACGCGACGAGTGTCGAAGC
GATCGCGAAAGCAGCCAATGAAGTCGGCGCTTGGGTTATTCACTACTCTACTGACTACGTATTTCCGGGGACCGGTG
AAATACCATGGCAGGAGGAGGATGCAACCGCACCGCTAAATGTTTACGGTGAAACCAAGTTAGCAGGAGAAAAAGCA
TTACAAGAGCATTGTGCGAAGCACCTTATTTTCCGGACCAGCTGGGTCTATGCAGGTAAAGGAAATAACTTCGCCAA
AACGATGTTGCGTCTGGCAAAAGAGCGTGAAGAATTAGCCGTTATTAATGATCAGTTTGGTGCGCCAACTGGCGCAG
AGTTGCTGGCTGATTGTACGGCACATGCCATTCGTGTGGCACTGAATAAACCGGAAGTCGCAGGTTTGTACCATCTG
GTAGCCAGTGGTACCACAACCTGGCACGATTATGCTGCGCTGGTTTTTGAAGAGGCGCGCAAAGCAGGCATTCCCCT
TGCACTCAACAAGCTCAACGCAGTACCAACAACAGTCTATCCTACACCAGCTCGTCGTCCACATAACTCTCGCCTTA
ATACAGAAAAATTTCAGCAGAACTTTGCGCTTGTCTTGCCTGACTGGCAGGTTGGTGTGAAACGCATGCTCAACGAA
TTATTTACGACTACAGCAATTTAATAGTTTTTGCATCTTGTTCGTGATGGTGGAACAAGATGAATTAAAAGGAATGA
TGGAATGAATACGCGTAAAGGTATTATTTTAGCGGGTGGTTCTGGTACACGTCTTTATCCTGTGACTATGGCTGTCA
GTAAACAGCTGTTACCGATTTATGATAAACCGATGATCTATTACCCGCTCTCTACACTGATGTTGGCGGGTATTCGC
GATATTTTGATTATCAGCACGCCACAGGATACTCCTCGTTTTCAACAACTGCTGGGTGATGGGAGCCAGTGGGGGCT
AAATCTTCACTACAAAGTGCAACCGAGTCCGGATGGTCTTGCGCAGGCATTTATCATCGGTGAAGAGTTTATCGGTG
GTGATGATTGTGCTTTGGTACTTGGTGATAATATCTTCTACGGTCACGACCTGCCTAAGTTAATGGATGCCGCTGTT
AACAAAGAAAGTGGTGCAACGGTATTTGCCTATCACGTTAATGATCCTGAACGCTATGGTGTCGTTGAGTTTGATAA
AAACGGTACTGCAATCAGCCTGGAAGAAAAACCGTTACAACCAAAAAGTAATTATGCGGTAACCGGGCTTTATTTCT
ATGATAACTACGTTGTGGAAATGGCGAAAAATCTTAAGCCTTCTGCCCGCGGTGAACTGGAAATTACCGATATTAAC
CGTATCTATATGGAACAGGGGCATTTATCTGTTGCCATGATGGGACGTGGATATGCCTGGCTGGACACGGGGACACA
TCAAAGTCTTATTGAAGCAAGCAACTTCATTGCCACCATTGAAGAGCGCCAGGGCTTGAAAGTTTCCTGCCCGGAAG
AAATTGCTTACCGTAAAGGGTTTATTGATGCTGAGCAGGTGAAAGTATTAGCTAAACCGCTGAAAAAAAATGCTTAT
GGTCAGTATCTGCTAAAAATGATTAAAGGTTATTAATAAAATGAATGTTATTAAAACAGAAATTCCAGATGTACTGA
TTTTTGAACCGAAAGTTTTTGGTGATGAGCGTGGTTTCTTTATGGAAAGCTTTAATCAGAAAGTTTTCGAAGAGGCT
GTAGGGCGGAAGGTTGAATTTGTTCAGGATAATCATTCTAAATCGTGTAAAGGTGTACTTAGAGGTTTACACTTTCA
GCTTCCTCCCTTTGAGCAGGCAAAATTAGTAAGGTGTATAGTTGGCGAGGTATTTGATGTTGCAGTAGACATTAGAC
CTAATTCTGAAACATTTGGTTCATGGGTTGGAGTAACTCTTTCGTCAGAAAATAAAAGGCAGCTATGGATTCCAGAA
GGATTCGCCCATGGTTTTTTAACTTTAAGTGATATTGCAGAGTTTGTTTATAAAACTAACAACTATTATTCTTTAAA
TCATGAAAGGGGAGTCATTTGGAACGATGAGGAAATTAACATTGCCTGGCCCTCTCAATCAGAGAAGATTCTGTCAC
AGAAAGATATTAATTTACCATCATTTAGATTTGTTCAAATGTTTAGCAAGTAGTGTTATCTTTACACTGCACATAGT
CATCATTTTTTATGCTTTAAGTAAATTATATTGCACATCTATAACACAAAGCGCAATAATATTTCGACCTGATGAAG
GTTTGTGGTTATTTATCTTTCTAGGCGTTTTTTATGACTAAAATAGTTGTGGTTTCTACAGCTCCAATATTCCCGAC
AAATAATGGGTACAAAAGTTCTGTATTAGGAAGAATTGATGAGTTATTAAATGAGGATAATGAGGTCGTTTTGATTG
AAATAAACCTTGAAAATGTTACGGAAAAGAAAGATGAATTAATACCAACAAGATTTAATAATATTCAAAGATATGAA
GTAAAAAAAATATCTAGATCATTTATTGCCGAGTTACAAATATTATTTGATATCAGAACTCGGTATGAACAATTATT
TTCTTCTGCTGACATTAGAGATAACATAAAAAAGATAATTGATTTAGAAAAACCTTCTATTATTATTGCTGAGTCTA
TATGGGCGTTGCAAGCATTGCCTATTGAAATTAGTGCGAGAATACACTGTGTTATTCATGATGTGGCAACTGATTTC
TTTAAAGAAATGTTTGTATCTCATAATGAGGTTGTACGAAAAATTTTGTTTTTTAATGATTACCTAAAGTTGAAAAT
TACTGAAGAAAATATTATCAAACGTTTGAGAGTTGAGCAATTTATCTTTCTGACAGAAGAAGATAAATGTTGGTATA
AAACAAGATACAATATTGATGAGGGTTGTTGTTCCTTAGCGAGCAATCATCTTTATGTAGAAAAGATTAAGAGAACT
ATCAATTTCCAAACCCCTTTCCTGCTTATTCCCGGTAGCATTGAATTTTCACAAAATTTTTACGGCTTAAATTGGTT
TATAAAAAATATATATCCTGGATTAAATAGGAAAATAAGAATAGTTGTAACAGGAAAGGCATCAGATAAAAAAATAA
AGATGTTAAACTGTGGAGAGGAAATTACCTTTACGGGAGAGCTTGACTTTTCCACATATAATAAACTTAGCTCAACA
TGCTTGTGTGTTATTGCACCGATTACAACGGGCACTGGAATTAAAATAAAAATATTAGAAGCTGTACAAAAAGGTAT
TCCTGTACTTACAACAAAATTTGCTTCAAAAGGAATATGTTCCGATTTATGTTTTTATTGCGAGGAGGATACTGACA
CAAACTTTGTCAATTTAATTAACAGTTTTCTTGAAACGACATTAAGAGTCCAAGAATGAATTTATTGCTTTTTTCAG
TCCTTGCGTTTGGTTTAATATTGGCTTTGGCCCATAATAATAAAAGTGGAGATATTAACGCATACTTAATGTTTTTT
CTCGTGGTCCTAATGGTATTAATATCAGGGCTGCGTATGAATGATAGTGATTATATCGAATACAGGAAAATGTATAA
TGAAGTGCCTATTTTATGTGACTTTAGTCTCGCATCTATAAGAGATATACATGGGGAGGTAGGCTATCTATTCTTAT
CATCAATCTTTAAAACTTTATGCTTGCCATTTCAATTATTTCTTTTTTTTATTGCTTTTTTATCACTCCTGCTTACA
TATTTTTCATTCAGAAAAATAAGTTTAATACCGATACTATCGTTAGTTTTTTATTTAAGCCATGCTTTTATAGTTAG
AGATTTGATTCAAATTAGGGCAGGATTAGCTGTTAGCATATCATTATATTCAATAATTAAATTTAAAGGAAATAAAA
GTATAATTACAGGAGTTTTATTTGCTTCTTTGATTCATTCTGGGGCGCTTATTATTGCTCTTTGTTATCCTTTTTTC
AAAAAAAAATACATAACATTAAAAATGATGTTGTTTTTATTTTTAGTGTCAATTATTTTTTCTTATTTGAATGGGCT
TAATTTATCGATACAACTCTTATCTCAATATAGTTTGCTTCCAACTGCAATTTCGAATTATGTTGGTTGGGAAGAAT
ATGATTATCGGGTGAGTATATTTACTAATCCGGTTTTTATTAAAGGTGTTTTTTTAATTGTCTTAATGCACAAATAT
GTACTTTCAGATATTAAAAATGAGAAAATTATAGTGCTTTATAACTTATATGTTTTAGGTGTATTAGCTATGGTTGC
ATTGAGTGGGATGGCTATTCTTTCAGGCCGTCTTTCATCCTTTCTGACACTAGGTGAAAGCATTTTAATTGTATATG
CTCTGTTCTACAAAAGAAATACACCTCTGGCGTTTCTAATTTTTTCTTTTTTAACAATTGTGCAATTAGGATATGAT
CTATTTATTTCTAATGTGCATCCTGAGCTTACTCTGATTATATTTGGGTGAATCTAAGTGAAAAATAATAAAATAGG
CATACTTATCTCTAAAATACAAAATCTTGGACCTGTGAATGTAGTACGAGGATTGATAAAAGAAAATAAAAAATATG
CTTTTACTGTTTTTTGTTTAACAAATAGCGTAGATAAAAATATATATGATGAGTTATGCTGTTTAGGAGCCAAGGTT
ATATTAATACCAGATGGTACTTGGTTCAGCAAAATTTTATTTGTGAGAAGTTTTTTAAAGGAACATCCACATAATAT
CTTACATTCACATGGGATCACGGCCGATATGTTTTCTTACTTTCTGAATGGCGTGAAAATATCTACTATTCACAATA
GACTAGATGAGGATTATATCCCATTATTTGGCGCGGTTAAAGGGAATGCTATATATTATCTTCATCGTTTTATATTA
CGAAGATTTAATCATATCGTTGCTTGCTCAGCAGCGGTCCAATCAAAACTGAAACAATCGAAAGTAAAAACTAAAAT
AACCACCATCCAGAATGGGATTGATATAACTAGGTTTAAGACACTTGAGTCTGATAAAAAAAAATTATTGAGGGAAA
AACACGGATTTGATAGTGAAAAAAGAATATTTATATATTGTGGCTCGTTATCATTAAGGAAAAATATTGCTTACCTC
TTGGAACACTTAGCCATCGAAGAAAATGATATATTTTTAATTCTAGGTGATGGTGAACTTTTTAGATATTGTAAGGA
TAAATATTCTAAAGATTTACGGTATATATTTATGGGGAAAGTTGAATGCCCTCTTGAATATTATCAATTATCAGATA
TTTTTGTTTCCGCTTCTTTATCGGAAGGGCTCCCCTTGGCACTATTAGAAGCTGCCTCTACTGGGTGCTATTTATAT
GTTAGCGATATAGAGCCCCATAGAGAAATTGCATCTCTATTAGGAGAGGAAAATATTTCTATGTTTAAAATTAAGGA
TGGATCATATAATTATTTGCAACCTAAAATAAAAAAAGCTGACTATAACGCTCTTTCTGACGATAAACTTTACAATA
TATCCGATAAAAAAATGTCAAATCTTTATGACAAACTTTTTGTTTCTTTATTAGAGCAGAGGCACTAATATAATGAT
TTATGTTTCGGTAATTTCTCATGGTCATTTCAAAACTCTTAAGGAATTAGGAGCAGTATCAAAATTAAATAATCACA
GCAGAATTAAAGTTATCATCAAAGATAATTTAGGAGAGAGCGAGCTTTTGGATTTTTGTCAGGAAAACAAAATAACT
TATTTAAGGTCTAAAGAGAAAAAAGGATTTGGAGAGAATAATAATGAAGTTTTTTCCTCTATATCCTCCTTAATTAC
TAAGGAAGATTTTTTTGTGGTTATGAATCCTGATATATATATTGAGTGCTCTGATCTATTAGATGTCGTAGATGAGT
GTGGTTCAGCGAATGTTAATCTAGCAACGATAAATTTATACAGGGATTTTGATAAAAAAACATATGATAACTCAGTA
AGGAAATTTCCCTCGGCAATTGATTTTTTTATGTCATTTTTATTTAAGAAAAATGACTGTGTAGTAAATAAGAACAA
AATAACGAAACCAACATATGTTGATTGGGCTGCAGGTTCTTTTCTAATATTTAATGCCTTCTTTTATTCAAAACTCA
ACGGATTCAACGAAAAGTATTTTATGTATTGCGAAGATATTGATATATGTTGGCGAGCTAAAAAACACTTCAATACT
TCAGTTTTATACTATCCATGCTATGCAGCAATTCATTTGGCACAATTTAACAATCGTAGGATTTTTAGTAGACATTT
CATTTGGCATATAAAAAGTATTATCCTTTTTTTATTATATAAAAATGGTATGCTGCGTTCTAGTAAGTTGCTTTAAT
GCTAATATTCTTTTAAGAGGTGAGAATGATACCTGTTATTTTGGCTGGTGGTTCGGGAAGTCGCTTGTGGCCACTTT
CACGAGAAAAGTTCCCCAAGCAGTTTTTAAAGTTGACTGGCAGTTTGACAATGTTGCAGTCAACATTGTCACGTCTT
AATAATTTAAATGCTGATGATTCAATAGTTATATGCAACGAAGAGCATAGATTTATTGTTGCAGAACAATTAAGAGA
GTTAGGCAAACTTTCAAATAACATTATTCTTGAACCCAAAGGTCGTAATACAGCCCCTGCTATAACACTCGCAGCAT
TAGCAGCAAAAAGAAAATTCGCTGATGAAGATCCATTGATTCTTATTTTAGCTGCAGATCACAACATCCAAGACGAA
CATGTTTTCTGTGAGGCAATTAATAAGGCGTCATCTTTAGCTAGTTATGGAAAACTAGTGACTTTTGGTATCGTTCC
ATTCAAACCTGAAACTGGGTATGGCTATATTCGTCGCGGTGATGAAGTGCCTGTAGATGAGCAGCATGCGGTGGCCT
TTGAAGTGGCGCAGTTTGTCGAAAAACCGAATCTGGAAACCGCGCAGGCCTATGTGGCAAGCGGCGAATATTACTGG
AACAGCGGTATGTTCCTGTTCCGTGCCGGACGCTATCTCGAAGAACTGAAAAAGTATCGTCCGGATATTCTCGATGC
CTGTGAAAAAGCGATGAGCGCCGTCGATCCGGATCTCGATTTTATTCGTGTGGATGAAGAGGCGTTTCTCGCTTGTC
CGGAAGAGTCGGTGGATTACGCGGTCATGGAATGCACGGCAGATGCCGTTGTGGTGCCGATGGATGCGGGCTGGAGC
GATGTCGGTTCCTGGTCTTCATTATGGGAGATCAGCGCCCACACCGCCGAGGGCAACGTTTGCCACGGCGATGTGAT
TAATCACAAAACTGAAAACAGCTATGTGTACGCCGAATCTGGCCTGGTCACCACCGTCGGGGTGAAAGATTTGGTGG
TAGTGCAGACCAAAGATGCAGTGCTGATTGCCGACCGTAATGCGGTGCAGGATGTGAAGAAAGTGGTCGAGCAGATC
AAAGCTGATGGTCGCCATGAGCATCGGGTGCATCGCGAAGTGTATCGTCCGTGGGGCAAATATGACTCTATCGACGC
GGGCGACCGCTACCAGGTGAAACGCATCACCGTGAAACCGGGCGAAGGTTTGTCGGTACAGATGCATTATCATCGCG
CGGAACACTGGGTGGTTGTCGCGGGAACGGCAAAAGTCACTATCAACGGTGATATCAAACTGCTTGGTGAAAACGAG
TCCATTTATATTCCGCTGGGGGCGATGCACTGCCTGGAAAACCCGGGGAAAATAGATTTAGAATTAATTGAAGTTCG
CTCTGGTGCATATCTTGAAGAAGATGATGTTATTAGATGTTATGATCGCTATGGACGAAAGTAATATATAATAATTA
TTTCAGAATTAGAAATGATAATTATAAGTTTTCGTCTGGATAAACAATAGATAGTATGGGTTGGAAAATATGAGTTC
TTTAACTTGTTTTAAAGCTTACGACATTCGCGGGAAATTAGGTGAAGAACTGAATGAAGATATCGCCTGGCGCATTG
GTCGCGCCTATGGCGAATTTCTCAAACCGAAAACCATTGTGTTAGGCGGTGATGTCCGTCTCACCAGCGAAACCTTA
AAACTGGCGCTGGCAAAAGGTTTACAGGATGCGGGCGTCGATGTGCTGGATATTGGCATGTCCGGCACCGAAGAGAT
TTATTTCGCCACGTTCCATCTCGGCGTGGATGGCGGCATTGAAGTTACCGCCAGCCATAATCCGATGGATTACAACG
GCATGAAGCTGGTGCGCGAAGGGGCTCGCCCGATCAGCGGTGATACCGGACTGCGCGACGTCCAGCGTCTGGCAGAA
GCTAACGACTTTCCTCCCGTCGATGAAACCAAACGCGGTCGCTATCAGCAAATCAATCTGCGTGACGCTTACGTTGA
TCACCTGTTCGGTTATATCAATGTCAAAAACCTTACGCCGCTCAAGCTGGTGATCAACTCCGGGAATGGCGCAGCGG
GTCCGGTGGTGGACGCTATCGAAGCCCGCTTTAAAGCCCTCGGCGCACCGGTGGAGTTAATCAAAGTGCATAACACG
CCGGACGGCAATTTCCCCAACGGTATTCCTAACCCGTTGCTGCCGGAATGTCGCGACGACACCCGCAATGCGGTCAT
CAAACACGGCGCGGATATGGGCATTGCCTTTGATGGCGATTTTGACCGCTGTTTCCTGTTTGACGAAAAAGGGCAGT
TTATTGAGGGCTACTACATTGTCGGCCTGCTGGCAGAAGCGTTCCTCGAAAAAAATCCCGGCGCGAAGATCATCCAC
GATCCACGTCTCTCCTGGAACACCATTGATGTGGTGACGGCCGCGGGCGGCACGCCGGTGATGTCGAAAACAGGACA
CGCCTTTATTAAAGAACGTATGCGCAAGGAAGACGCCATCTACGGTGGCGAAATGAGCGCTCACCATTACTTCCGCG
ATTTCGCTTACTGTGACAGCGGCATGATCCCGTGGCTGCTGGTCGCCGAACTGGTGTGCCTGAAAGGAAAAACGCTG
GGCGAACTGGTGCGCGACCGGATGGCGGCGTTTCCGGCAAGCGGTGAGATCAACAGAAAACTGGCGCACCCTGTTGA
GGCGATTAACCGCGTGGAACAGCATTTTAGCCGTGAGGTGCTGGCGGTGGATCGCACCGATGGCATCAGCATGACCT
TTGCCGACTGGCGCTTTAACCTGCGCTCTTCCAACACCGAACCGGTGGTGCGCCTGAATGTGGAATCTCGCGGTGAT
GTTCAGGTTATGGTAATCCATACTCAAGAAATATTATCAATTTTGACGTCATAAAGAATAAGCCCTGACAAGTTAGG
GCTTAATTAATATATATTTTTTTTGAATTGGGGATTTGTGGTAAGATTTTTAATATGTTATTTAATGTGGTTGAATT
AATGTTGACTGGAAAATAATAATGAGAACGAAAAAAGCATTACACAACTTTAAAGTTGATTTATTAATTACTTTTTT
ATTGGTTTTGCTAGGGTTTTATATTCGAACTGTTTTTGTTTCAAAAATGGGAAGTGATATTACTGGAGTGATGTTAC
TATTCACACAGTTGACAGCATATCTCAATTTGGCAGAATTAGGTATTGGAATTGCAGCTGCCAGCGTATTATATAAA
CCGCTCAGCGAGAATGAATACAATAAAATAACTTACATAATATCTTTGCTCTCAGTCATATACAAATATATATTTGT
GTTTGTTTTGATTCTTGGCGTTGTTATAGGTATCTGTATTTATTACTTTATTGATTCTGTAAAGGTTGTAAATGGCG
TTTTTTTATATTGGGCTTTGTTCGTTTTTAATACATCGTTGACATATAGTTATGCTAAATACTCCACATTATTAACT
GCTAATCAGCGGTACTCAGCAGTAAGAAAAATTCAAGGTGGCGGAAAAGTTATAATAATTGTATTTCAGATATTAAT
TTTGTGCTTTACGCAAAGTTTCATACTTTATTTGTTAGTTGAGACTTTAGGTATTTTTTCTCAATATTTGATTTTTA
AAAAAATAATTGGGAACGGAAATCAATATCTCAGTAATGAGGTTTTACTTATTGAAAGCGATAAACTTTTGATAAAA
AAAGAATTAAAAATAAGAATAAAAAATATGTTCTTCCATAAAATAGGTGCTGTGCTTGTCCTTAATACAGACTACCT
GCTTGTATCAAAGTTTCTGACATTAAGTTATGTGACAATTTTTGGCAGCTATATGATGGTATTTCAGATAGTAACTG
TTTTGATGTCAAGTTTTGTTAATGCTATTACTGCAGGAATGGGTAATTACTTAATTAATAAAAGTAATTTAGAAATT
AAGGAAATTACACGTCAATTTTATGTGATATTTATCGCCTTTGCAACATTCATATCACTAAATATGTTTTTTCTTGT
TAATGATTTTATCGCAAAATGGATAGGTGTTAATTATACATTAAGTAACACCCTAGTTGCATTAATGATTGTTAACG
TATTCATTAGTGTTGTCAGGGTACCTTCTGATATATTAAAAAACGCAAGTGGACATTTTGGTGATATTTATTATCCA
TTATTAGAAGGTGTGCTGAATATTACGATATCCATCATTTTGGCTATCATTATTGGATTACCTGGCATTATTATAGG
GACAATAGTATCTAACTTAATAGTAATAATGCTTGCGAAACCATTATATCTTTACTCTAAGTTATTTAATCTTAGAA
ATCCGACGAGGGTTTATTTTGAATTTATTTCTCGGCCTATGTTATATTCATTATGTGTGATTGGGGTGAGCTATTTA
TTGCGCGATGAAATATATTCATTTAAAGTAAGTACATGGTTGGATTTTATTAACAAGCTACTCTTAGTCTCTACTCC
TAGCATATTGGTAATATGTGCTATTTTCTCTACGGATAGTGACTTTAGATTATTTTTCAGAAAAATTATATATGTGA
TTATGAAGAAATAAAAATTTCGAAAATGTATTAATCGAAATTATGCAACGAGCTTTATTTTTATAAATGATATGTGA
TCTTTTCGCGAATAGGAGTAAGGATCCGTGTAGGCTGGAGCTGCTTCGAAGTTCCTATACTTTCTAGAGAATAGGAA
CTTCGGAATAGGAACTAAGGAGGATATTCATATGGATAAAGCCGTAAGCATATAAGCATGGATAAGCTATTTATACT
TTAATAAGTACTTTGTATACTTATTTGCGAACATTCCAGGCCGCGAGCATTCAGCGCGGTGATCACACCTGACAGGA
GTATGTAATGTCCAAGCAACAGATCGGCGTAGTCGGTATGGCAGTGATGGGACGCAACCTTGCGCTCAACATCGAAA
GCCGTGGTTATACCGTCTCTATTTTCAACCGTTCCCGTGAGAAGACGGAAGAAGTGATTGCCGAAAATCCAGGCAAG
AAACTGGTTCCTTACTATACGGTGAAAGAGTTTGTCGAATCTCTGGAAACGCCTCGTCGCATCCTGTTAATGGTGAA
AGCAGGTGCAGGCACGGATGCTGCTATTGATTCCCTCAAACCATATCTCGATAAAGGAGACATCATCATTGATGGTG
GTAACACCTTCTTCCAGGACACTATTCGTCGTAATCGTGAGCTTTCAGCAGAGGGCTTTAACTTCATCGGTACCGGT
GTTTCTGGCGGTGAAGAGGGGGCGCTGAAAGGTCCTTCTATTATGCCTGGTGGCCAGAAAGAAGCCTATGAATTGGT
AGCACCGATCCTGACCAAAATCGCCGCCGTAGCTGAAGACGGTGAACCATGCGTTACCTATATTGGTGCCGATGGCG
CAGGTCACTATGTGAAGATGGTTCACAACGGTATTGAATACGGCGATATGCAGCTGATTGCTGAAGCCTATTCTCTG
CTTAAAGGTGGCCTGAACCTCACCAACGAAGAACTGGCGCAGACCTTTACCGAGTGGAATAACGGTGAACTGAGCAG
TTACCTGATCGACATCACCAAAGATATCTTCACCAAAAAAGATGAAGACGGTAACTACCTGGTTGATGTGATCCTGG
ATGAAGCGGCTAACAAAGGTACCGGTAAATGGACCAGCCAGAGCGCGCTGGATCTCGGCGAACCGCTGTCGCTGATT
ACCGAGTCTGTGTTTGCACGTTATATCTCTTCTCTGAAAGATCAGCGTGTTGCCGCATCTAAAGTTCTCTCTGGTCC
GCAAGCACAGCCAGCAGGCGACAAGGCTGAGTTCATCGAAAAAGTTCGTCGTGCGCTGTATCTGGGCAAAATCGTTT
CTTACGCCCAGGGCTTCTCTCAGCTGCGTGCTGCGTCTGAAGAGTACAACTGGGATCTGAACTACGGCGAAATCGCG
AAGATTTTCCGTGCTGGCTGCATCATCCGTGCGCAGTTCCTGCAGAAAATCACCGATGCTTATGCCGAAAATCCACA
GATCGCTAACCTGTTGCTGGCTCCGTACTTCAAGCAAATTGCCGATGACTACCAGCAGGCGCTGCGTGATGTCGTTG
CTTATGCAGTACAGAACGGTATTCCGGTTCCGACCTTCTCCGCAGCGGTTGCCTATTACGACAGCTACCGTGCTGCT
GTTCTGCCTGCGAACCTGATCCAGGCACAGCGTGACTATTTTGGTGCGCATACTTATAAGCGTATTGATAAAGAAGG
TGTGTTCCATACCGAATGGCTGGATTAA
(Example CRM197 sequence)
SEQ ID NO: 20
GADDVVDSSKSFVMENFSSYHGTKPGYVDSIQKGIQKPKSGTQGNYDDDWKEFYSTDNKYDAAGYSVDNENPLSGKA
GGVVKVTYPGLTKVLALKVDNAETIKKELGLSLTEPLMEQVGTEEFIKRFGDGASRVVLSLPFAEGSSSVEYINNWE
QAKALSVELEINFETRGKRGQDAMYEYMAQACAGNRVRRSVGSSLSCINLDWDVIRDKTKTKIESLKEHGPIKNKMS
ESPNKTVSEEKAKQYLEEFHQTALEHPELSELKTVTGTNPVFAGANYAAWAVNVAQVIDSETADNLEKTTAALSILP
GIGSVMGIADGAVHHNTEEIVAQSIALSSLMVAQAIPLVGELVDIGFAAYNFVESIINLFQVVHNSYNRPAYSPGHK
TQPFLHDGYAVSWNTVEDSIIRTGFQGESGHDIKITAENTPLPIAGVLLPTIPGKLDVNKSKTHISVNGRKIRMRCR
AIDGDVTFCRPKSPVYVGNGVHANLHVAFHRSSSEKIHSNEISSDSIGVLGYQKTVDHTKVNSKLSLFFEIKS

The embodiments described herein are intended to be merely exemplary, and those skilled in the art will recognize, or be able to ascertain using no more than routine experimentation, numerous equivalents to the specific procedures described herein. All such equivalents are considered to be within the scope of the present invention and are covered by the following claims.

All references (including patent applications, patents, and publications) cited herein are incorporated herein by reference in their entirety and for all purposes to the same extent as if each individual publication or patent or patent application was specifically and individually indicated to be incorporated by reference in its entirety for all purposes.

1-7. (canceled)

8. A composition comprising an E. coli O1 antigen polysaccharide covalently linked to a carrier protein, an E. coli O2 antigen polysaccharide covalently linked to a carrier protein, an E. coli O4 antigen polysaccharide covalently linked to a carrier protein, an E. coli O15 antigen polysaccharide covalently linked to a carrier protein, an E. coli O16 antigen polysaccharide covalently linked to a carrier protein, an E. coli O18 antigen polysaccharide covalently linked to a carrier protein, an E. coli O25 antigen polysaccharide covalently linked to a carrier protein, an E. coli O75 antigen polysaccharide covalently linked to a carrier protein, and an E. coli O6 antigen polysaccharide covalently linked to a carrier protein, wherein:
(i) the E. coli O1 antigen is a O1A polysaccharide comprising the structure of Formula (O1A): 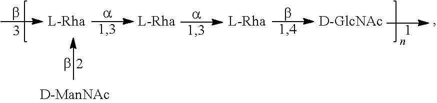
(ii) the E. coli O2 antigen is a O2 polysaccharide comprising the structure of Formula (O2): 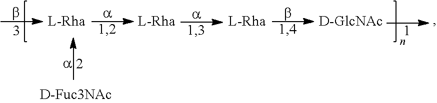
(iii) the E. coli O4 antigen is a O4-Glc+ polysaccharide comprising the structure of Formula (O4-Glc+): 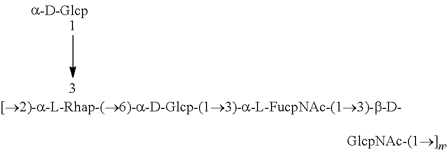
(iv) the E. coli O6 antigen is a O6A polysaccharide comprising the structure of Formula (O6A): 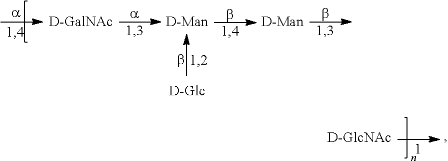
(v) the E. coli O15 antigen is a O15 polysaccharide comprising the structure of Formula (O15):
[→2)-3-D-Galp-(1→3)-α-L-FucpNAc-(1→3)-β-D-GlcpNAc-(1→]ₙ,
(vi) the E. coli O16 antigen is a O16 polysaccharide comprising the structure of Formula (O16): 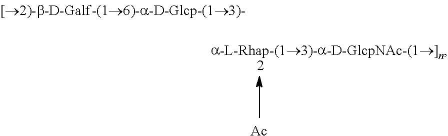
(vii) the E. coli O18 antigen is a O18A polysaccharide comprising the structure of Formula (O18A): 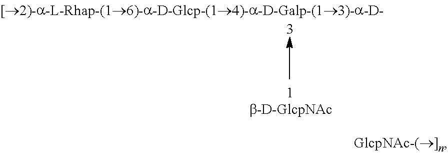
(viii) the E. coli O25 antigen is a O25 polysaccharide comprising the structure of Formula (O25B): 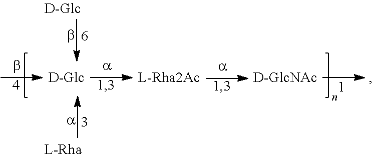
and
(ix) the E. coli O75 antigen is a O75 polysaccharide comprising the structure of Formula (O75): 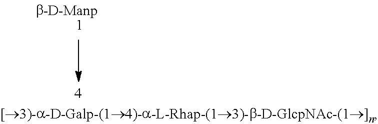
wherein each n is independently an integer of 1 to 100; wherein the weight ratio of the E. coli antigen polysaccharides O1:O2:O4:O6:O15:O16:O18:O25:O75 is about 1:1:1:1:1:1:1:2:2 and further comprising:
(x) an E. coli O8 antigen polysaccharide covalently linked to a carrier protein, wherein the O8 polysaccharide comprises the structure of Formula (O8):
α-D-Manp3Me-(1→[3)-β-D-Manp-(1→2)-α-D-Manp-(1→2)-α-D-Manp-(1→]ₙ
wherein n is an integer of 1 to 100.

9-22. (canceled)

23. The composition of claim 8, wherein the weight ratio of the E. coli antigen polysaccharides O1:O2:O4:O6:O8:O15:O16:O18:O25:O75 is about 1:1:1:1:1:1:1:1:2:2.

24. The composition of claim 23, wherein the concentration of the E. coli O75 antigen polysaccharide is from about 16 to about 32 μg/ml.

25. The composition of claim 24, wherein the composition consists of the E. coli O1, O2, O4, O8, O15, O16, O18, O25, O75 and O6 antigen polysaccharides.

26. The composition of claim 24, wherein each of the carrier proteins is a detoxified exotoxin A of Pseudomonas aeruginosa or CRM₁₉₇.

27. The composition of claim 24, wherein each of the carrier proteins is a detoxified exotoxin A of Pseudomonas aeruginosa comprising 1 to 10 glycosylation consensus sequences having the amino acid sequence of SEQ ID NO: 2.

28. The composition of claim 24, wherein each of the carrier proteins comprises the amino acid sequence of SEQ ID NO: 3.

29. The composition of claim 24, wherein each of the antigen polysaccharides is covalently linked to its carrier protein by bioconjugation or conjugation.

30. The composition of claim 24, wherein each n is independently an integer of 5 to 30.

31. The composition of claim 28, wherein each n is independently an integer of 5 to 30.

32. The composition of claim 31, wherein the weight ratio of the E. coli antigen polysaccharides O1:O2:O4:O6:O8:O15:O16:O18:O25:O75 is 1:1:1:1:1:1:1:1:2:2.

33. The composition of claim 30, wherein the carrier protein is CRM₁₉₇.

34. The composition of claim 23, wherein each n is independently an integer of 3 to 50, each carrier protein comprises the amino acid sequence of SEQ ID NO: 3, each of the E. coli antigen polysaccharides are covalently linked to its carrier protein by bioconjugation to an Asn residue in a glycosylation site in its carrier proteins, and the O75 antigen polysaccharide concentration is from about 16 to about 32 μg/ml.

35. The composition of claim 34, wherein the weight ratio of the E. coli antigen polysaccharides O1:O2:O4:O6:O8:O15:O16:O18:O25:O75 is 1:1:1:1:1:1:1:1:2:2

36. The composition of claim 35 wherein the O75 antigen polysaccharide concentration is about 16 μg/ml.

37. The composition of claim 35, wherein the O75 antigen polysaccharide concentration is 16 μg/ml.

38. The composition of claim 35, wherein the O75 antigen polysaccharide concentration is about 32 μg/ml.

39. The composition of claim 35, wherein the O75 antigen polysaccharide concentration is 32 μg/ml.

40. The composition of claim 34, wherein each polysaccharide/carrier protein ratio is about 0.1 to 0.5.

41. The composition of claim 35, wherein each polysaccharide/carrier protein ratio is about 0.1 to 0.5.

42. The composition of claim 36, wherein each polysaccharide/carrier protein ratio is about 0.1 to 0.5.

43. The composition of claim 37, wherein each polysaccharide/carrier protein ratio is about 0.1 to 0.5.

44. The composition of claim 38, wherein each polysaccharide/carrier protein ratio is about 0.1 to 0.5.

45. The composition of claim 39, wherein each polysaccharide/carrier protein ratio is about 0.1 to 0.5.


SEQUENCE LISTING (20 sequences)

SEQ ID NO: 1-2 - skipped (2 entries)

SEQ ID NO: 3 - protein, 652 aa, synthetic construct
  REGION 1..652: note = EPA carrier protein comprising 4 glycosylation consensus sequences (EPA-4)
GSGGGDQNATGSGGGKLAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKDNNNSTPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAKDQNRTKGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKLGSGGGDQNAT

SEQ ID NO: 4 - protein, 421 aa, synthetic construct
  REGION 1..421: note = O4 GtrS amino acid sequence
MNNLIMNNWCKLSIFIIAFILLWLRRPDILTNAQFWAEDSVFWYKDAYENGFLSSLTTPRNGYFQTVSTFIVGLTALLNPDYAPFVSNFFGIMIRSVIIWFLFTERFNFLTLTTRIFLSIYFLCMPGLDEVHANITNAHWYLSLYVSMILIARNPSSKSWRFHDIFFILLSGLSGPFIIFILAASCFKFINNCKDHISVRSFINFYLRQPYALMIVCALIQGTSIILTFNGTRSSAPLGFSFDVISSIISSNIFLFTFVPWDIAKAGWDNLLLSYFLSVSILSCAAFVFVKGTWRMKVFATLPLLIIIFSMAKPQLTDSAPQLPTLINGQGSRYFVNIHIAIFSLLCVYLLECVRGKVATLFSKIYLTILLFVMGCLNFVITPLPNMNWREGATLINNAKTGDVISIQVLPPGLTLELRKK

SEQ ID NO: 5 - protein, 1266 aa, synthetic construct
  REGION 1..1266: note = Example O4 gtrS nucleic acid sequence
ATGAATAATTTAATTATGAATAACTGGTGTAAATTATCTATATTTATTATTGCATTTATTTTGCTATGGCTTAGAAGGCCGGATATACTCACAAACGCACAATTTTGGGCAGAAGATTCCGTTTTCTGGTATAAGGACGCCTATGAGAACGGATTCTTAAGTTCACTAACAACGCCTAGGAATGGGTATTTCCAGACTGTTTCTACATTTATAGTTGGTCTGACTGCTTTATTAAATCCAGATTATGCACCTTTTGTTTCTAATTTTTTTGGCATAATGATTCGCTCAGTAATTATATGGTTTTTATTTACAGAAAGATTCAACTTCCTCACATTGACTACTAGGATTTTCTTATCTATTTATTTTCTATGCATGCCTGGATTGGATGAAGTTCATGCAAATATAACAAATGCACATTGGTATTTGTCATTATATGTATCAATGATCCTGATAGCTCGCAATCCAAGTTCAAAATCATGGAGGTTTCATGATATATTCTTTATCTTGCTATCCGGGCTCAGTGGCCCATTTATAATTTTCATTTTAGCAGCTTCATGCTTTAAATTTATAAATAATTGTAAAGATCATATTAGTGTAAGATCTTTCATAAATTTCTACTTGCGTCAGCCATACGCATTAATGATTGTTTGCGCTTTAATTCAAGGAACTTCTATAATTCTAACTTTCAATGGCACACGTTCCTCAGCACCGCTAGGATTCAGTTTTGATGTGATTTCGTCTATTATATCATCGAATATTTTTTTATTTACATTTGTCCCATGGGATATTGCAAAGGCTGGGTGGGATAATTTACTGTTATCTTATTTTTTGTCTGTTTCGATTTTGTCGTGTGCGGCCTTTGTTTTTGTTAAAGGTACGTGGCGAATGAAAGTATTTGCAACTTTACCATTGCTAATTATAATATTTTCAATGGCAAAACCACAATTGACAGACTCGGCACCTCAATTGCCAACACTTATTAATGGGCAAGGTTCAAGATACTTCGTAAATATACATATTGCGATATTCTCTTTGCTATGTGTTTACTTACTTGAGTGCGTCAGGGGGAAAGTGGCAACTTTATTTTCCAAAATATACTTAACAATTTTGCTATTCGTGATGGGATGTTTGAATTTTGTTATCACCCCACTCCCAAACATGAACTGGAGGGAAGGTGCTACTTTGATTAATAATGCAAAAACTGGTGATGTCATTTCGATTCAAGTGCTACCACCTGGCCTAACACTTGAACTAAGGAAAAAATAA

SEQ ID NO: 6 - protein, 713 aa, synthetic construct
  REGION 1..713: note = Example PglB sequence ('wild-type')
MLKKEYLKNPYLVLFAMIILAYVFSVFCRFYWVWWASEFNEYFFNNQLMIISNDGYAFAEGARDMIAGFHQPNDLSYYGSSLSALTYWLYKITPFSFESIILYMSTFLSSLVVIPTILLANEYKRPLMGFVAALLASIANSYYNRTMSGYYDTDMLVIVLPMFILFFMVRMILKKDFFSLIALPLFIGIYLWWYPSSYTLNVALIGLFLIYTLIFHRKEKIFYIAVILSSLTLSNIAWFYQSAIIVILFALFALEQKRLNFMIIGILGSATLIFLILSGGVDPILYQLKFYIFRSDESANLTQGFMYFNVNQTIQEVENVDLSEFMRRISGSEIVFLFSLFGFVWLLRKHKSMIMALPILVLGFLALKGGLRFTIYSVPVMALGFGFLLSEFKAIMVKKYSQLTSNVCIVFATILTLAPVFIHIYNYKAPTVFSQNEASLLNQLKNIANREDYVVTWWDYGYPVRYYSDVKTLVDGGKHLGKDNFFPSFALSKDEQAAANMARLSVEYTEKSFYAPQNDILKTDILQAMMKDYNQSNVDLFLASLSKPDFKIDTPKTRDIYLYMPARMSLIFSTVASFSFINLDTGVLDKPFTFSTAYPLDVKNGEIYLSNGVVLSDDFRSFKIGDNVVSVNSIVEINSIKQGEYKITPIDDKAQFYIFYLKDSAIPYAQFILMDKTMFNSAYVQMFFLGNYDKNLFDLVINSRDAKVFKLKI

SEQ ID NO: 7 - protein, 120 aa, Escherichia coli
MLKLFAKYTSIGVLNTLIHWVVFGVCIYVAHTNQALANFAGFVVAVSFSFFANAKFTFKASTTTMRYMLYVGFMGTLSATVGWAADRCALPPMITLVTFSAISLVCGFVYSKFIVFRDAK

SEQ ID NO: 8 - protein, 306 aa, Escherichia coli
MKISLVVPVFNEEEAIPIFYKTVREFEELKSYEVEIVFINDGSKDATESIINALAVSDPLVVPLSFTRNFGKEPALFAGLDHATGDAIIPIDVDLQDPIEVIPHLIEKWQAGADMVLAKRSDRSTDGRLKRKTAEWFYKLHNKISNPKIEENVGDFRLMSRDVVENIKLMPERNLFMKGILSWVGGKTDIVEYVRAERIAGDTKFNGWKLWNLALEGITSFSTFPLRIWTYIGLVVASVAFIYGAWMILDTIIFGNAVRGYPSLLVSILFLGGIQMIGIGVLGEYIGRTYIETKKRPKYIIKRVKK

SEQ ID NO: 9 - DNA, 14440 bp, synthetic construct
  misc_feature 1..14440: note = example O4 rfb locus nucleotide sequence - O4-EPA production strain BVEC-L-00684f
  source 1..14440: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagtgaaaatacttgttactggtggcgcaggatttattggttcagctgtagttcgtcacattataaataatacgcaggatagtgttgttaatgtcgataaattaacgtacgccggaaaccgggaatcacttgctgatgtttctgattctgaacgctatgtttttgaacatgcggatatttgcgatgcacctgcaatggcacggatttttgctcagcatcagccggatgcagtgatgcacctggctgctgaaagccatgttgaccgttcaattacaggccctgcggcatttattgaaaccaatattgttggtacttatgtccttttggaagccgctcgcaattactggtctgctcttgatagcgacaagaaaaatagcttccgttttcatcatatttctactgacgaagtatatggtgatttgcctcatcctgacgaggtaaataatacagaagaattacccttatttactgagacaacagcttacgcgccaagcagcccttattccgcatccaaagcatccagcgatcatttagtccgcgcgtggaaacgtacctatggtttaccgaccattgtgactaattgctctaacaattatggtccttatcatttcccggaaaaattgattccattggttattctcaatgctctggaaggtaaagcattacctatttatggtaaaggggatcaaattcgcgactggctgtatgttgaagatcatgcgcgtgcgttatataccgtcgtaaccgaaggtaaagcgggtgaaacttataacattggtgggcacaacgaaaagaaaaacatagatgtagtgctcactatttgtgatttgctggatgagattgtaccgaaagagaaatcttatcgtgagcaaatcacttatgttgccgatcgtccgggacacgatcgccgttatgcgattgatgctgagaatattggtcgcgaattgggatggaaaccacaggaaacgtttgagagcgggattcggaagacagtggaatggtatctgtccaatacaaaatgggttgataatgtgaaaagtggtgcctatcaatcgtggattgaagagaactatgagggccgccagtaatgaatatcctcctttttggcaaaacagggcaggtaggttgggaactacagcgtgctctggcacctctgggtaacttgattgctcttgatgttcattccactgattattgtggcgatttcagtaaccccgaaggtgtggctgaaaccgtcaaaaaaattcgcccagatgttattgttaatgctgctgctcataccgcggtagataaggctgagtcagaaccagaatttgcacaattactcaatgcgaccagcgttgaagcaattgcaaaagcggctaatgaagttggggcttgggtaattcattactcaactgactacgtcttccctggaaatggcgacatgccatggctcgagactgatgtaaccgctccgctcaatgtttatggcaaaaccaaattggctggagaaagagcattacaagaacattgcgcaaagcatcttattttccgtaccagctgggtatatgcaggtaaaggaaataactttgccaaaacaatgttacgtctggcaaaagagcgcgaagaactggctgtgataaacgatcagtttggcgcaccaacaggtgctgaattgctggctgattgcaccgctcatgccattcgcgtggcattaaaaaaaccagaagttgctggcttgtaccatctggtagcaaatggcacaacaacctggcacgattacgccgcgctagtattcgaagaagcccgtaaagcagggattgaccttgcacttaacaaactcaacgccgtaccaacaacggcttatcctactccagcccgccgtcctcataattctcgcctcaataccgaaaagtttcagcagaactttgcgcttgtcttgcctgactggcaggtgggcgtgaaacgtatgctcaacgaattatttacgactacggcaatttaacaaatttttgcatctcgctcatgatgccagagcgggatgaattaaaaggaatggtgaaatgaaaacgcgtaaaggtattattctggctggtggttccggcactcgtctttatcctgtgacgatggcagtgagtaaacaactgctgccgatttatgataagccgatgatttattatccgctttcaacgcttatgttagcgggtattcgcgatattcttattatcagtacgccacaggatacaccgcgtttccaacaattgttgggggacgggagtcagtgggggcttaatctacagtataaagtacaaccgagtccggatggcctggcgcaagcgtttattattggtgaagactttattggtggtgatgattgtgcactcgtacttggcgataatatcttctatggacacgacttgccgaaattaatggaagctgctgttaacaaagaaatcggtgcaacggtatttgcttatcacgtcaatgatcctgaacgttatggtgtcgtggagtttgataataacggtactgcaattagcctggaagaaaaaccgctggaaccaaaaagtaactatgcggttactgggctttatttctatgacaatgatgttgtagaaatggcgaaaaaccttaagccttctgcccgtggcgaactggaaattaccgatattaaccgtatttatatggagcagggacgtttgtctgtcgctatgatggggcgtggttatgcctggttggatactggtacacatcaaagtcttattgaagcaagtaacttcattgccaccattgaagagcgtcagggattaaaggtatcttgcccggaagagattgcttaccgtaaagggtttattgatgctgagcaggtgaaagtattagccgaaccgctgaagaaaaatgattatggtcagtatctgctaaaaatgattaaaggttattaataaaatgaacgtaattaaaactgaaattcctgatgtgctgatttttgaaccaaaagtttttggtgatgaacgtggcttcttttttgagagttttaaccagaaagtatttgaagaagctgtaggacggaaggttgaatttgttcaggataaccattctaagtctaaaataaatgtattgcgtgggatgcattatcaaacacaaaatactcaaggaaaactggttcgggtaatttctggttcagtatatgatgttgccgtagatttaagagaaaaatcaaagacatttggcaaatgggtgggtgtagaattatctgggaataataaaagacaattgtggatccccgaaggttttgcccatggtttttatgtgttggaggagaataccgaatttgtttataaatgtaccgatacttataaccctgctcatgaacacacattgctatggaatgatccaactatcaatataagttggccaatcatacaaaactgcaagccaattatttctgaaaaagatgctaatggacatcttttttcacataaaacctatttctgaaatgcaatattatgagtttaattagaaacagtttctataatattgctggttttgctgtgccgacattagttgcagtccctgctttggggattcttgccaggctgcttggaccggagaattttggacttttcacactagcattcgctttgataggatatgcaagtattttcgacgccgggattagtcgagctgtaatcagagaaatcgctctttatcgagaaagtgaaaaagagcaaatacaaattatttcgacagcaagtgtaatcgtactattcttaggggtggttgcagctttgttactttattttagtagtaataaagttgttgagttattgaatgttagttccgtttatattgaaacagcagtgcgtgcattctctgttatttcatttataatacctgtgtatctgattaaccagatttggcttggttatctggaagggctagaaaaatttgcaaatataaatgttcagagaatgatttctagcacaagcttggctatattaccagtgatattttgttattacaatccctcgttgctttatgctatgtatgggttggtggttgggcgtgtgatttcatttttgattagcgcaataatttgtcgagatattattcttaaaagtaaactttactttaatgtggcaacttgcaatcgtcttatctcttttggtggatggataacagttagtaatatcataagcccaatcatggcatatttcgaccgctttatcatctctcatattatgggggcttcgagaattgcattttatacagcgccctcagagggtgtatcaaggttaattaatatcccatatgctttggcaagagctctatttcctaaattggcatatagcaataatgatgatgaacgaaaaaaattacaactacagagctacgcaattataagcattgtatgtctacccatagttgttattggtgtcatttttgcctcattcataatgacaacatggatgggacctgattatgccttagaagcagcaactatcatgaaaatacttcttgctggttttttctttaactctttagcgcaaataccttatgcatacttgcaatctatcggaaagtcaaaaattaccgcatttgtgcatctcatagaacttgcgccatacttattattattgtattacttcacaatgcatttcggcataattggcacggcaatcgcttggtcacttagaacattttgtgattttgttatactactttcgatatcgagaagaaaatgattgcggttgatattgcgcttgcaacctacaatggtgctaattttattcggcaacagattgaatctatccagaaacaaacttatagaaattggcgtcttataataagtgatgataactcgagtgatgatactgttgatattattaaggatatgatgtctaacgacagtcgtatctatttggtaggaaataaaagacaaggaggggttattcagaactttaattatgctctttcacaaactacatctgaaattgtgttactatgtgaccaggatgacatttggccggaggagcgtctggaaattcttatagataaatttaaggccttgcagcgtaatgattttgttccggcaatgatgtttactgatttgaaattagtagacgaaaataattgtttgattgcagaaagtttttatcgaacgaataatattaatccacaagataatctgaaaaataataatcttctctggcgttcaacggtatatggctgtacttgcatcatgaataagaaacttgttgatattgcattgcctatacctacatatgcacatatgcatgatcaatggttggcattattagcgaagcaatatggtaacattttttatttcgactatgcgtctgttcgttataggcaacattctacaaatgttgttggtggtagaaataaaacgccatttcaaaaatttaattccatacaaaaaaacctaaaaaggattaatttgctagtggatagaactgttgctttaattaaatcaaataacgatttctatccagggaataaaatggaaaataaaattgattacttaaaatttggagtgaatgaagtattaccttatctttttaaaggaaacaagaaagttttttcactttgtgtattaattagtttggcattacaaaaatgatatatttattatttttttttgcactgtttatgatctgtacgtttttaacacacaggcgacaggcattatatgttgtatctgcgttagtatttctttttttggctttaacctatccatcaggaggggactggataggttattttctccattatgactgcatggttaatgagcagtgtaataatggttttataatgtttgaacctggatatgaattaattgtttccttatttggatatttgggatttcagacaattattatttttatagccgctgtaaatgtaattctaatattaaattttgcaaagcattttgaaaacggaagttttgttattgttgcgataatgtgcatgttcctttggagtgtttatgttgaggcgattagacaggctctggccttatctatagttatatttgggattcattctctttttttgggtagaaaaaggaaatttataacattagtattatttgcgtcaactttccatataactgctttgatttgttttcttctaatgactcctctattttcaaagaaattaagcaagataataagttatagcctattaattttcagtagcttctttttcgctttttctgaaaccatattaagtgcactccttgcaattttgccagaaggatccattgccagtgaaaaattaagtttttacttagcaaccgagcaatacaggccacagttatctattgggagtggcactattcttgacattatacttatttttctgatatgtgtaagttttaaacgaataaagaaatatatgctcgctaattataatgctgcaaatgagatattgcttattggttgctgtctttatatttctttcggtatttttatcgggaaaatgatgccagttatgactcgcattggttggtatggttttccatttgttatagtacttctttatattaacttgggttattcagaatattttaagaggtatataaataaaagagggtgtgggtatagcaaattattaattgctttttattttttgctacaaattttgcgaccattaacatatgattatagctattataatataatgcaccaggatactttgctgaataggtttgatgcattagatgatgcatcattaagacaatcagcgaagagaaaatgtttcgatttgggaaagataggatatggtttcttatgtagtatataatatcctgcattcattcggataatttcctatggaagtgtcctttgctctgtctgtcctcatttgttgaaattttatgttaataagaagctttagataaccacttaggaactgtatgtttgatctgtccaaaaattatattattgtaagtgcgacggcgctggcttccggaggtgcattaactatattaaagcaatttataaaacatgcatcacaaaattcaaatgactatattatgtttgtatctgcgggattggagttgccggtctgtgataacatcatttacatagaaaacacaccaaaaggatggttgaaaagaatatattgggattggttcggttgtcggaagtttatctcggaacataagattaacgttaagaaagtaatttctctacaaaattccagtttgaatgttccttacgaacagattatttacttgcaccagccaattccttttagtaaagttgattcttttttaaaaaatatcacatccgataacgtaaagctttttttatataaaaagttttattcctattttatatttaaatatgtgaatgccaatacaaccatcgtagtgcaaacgaattggatgaaaaaaggagtgctggagcaatgtgataaaattagtaccgaaagggtccttgttataaaacctgatatcaaagcatttaataatactaattttgatgtagatatggatgtatctgcaaaaacactcttatatccagcgacaccacttacctataaaaatcatttggtcattctgaaggcgttggttattttaaagaaaaagtattttatagatgatctgaaattccaagtgacttttgaaaagaataggtacaaaaattttgataagtttgtgcaattaaataacttaagcaaaaacgttgattatctcggcgttctttcatactcgaacttgcaaaaaaaatatatggcggcatctttaatcgtttttcctagctatatcgaatcatatgggttaccactcatcgaagctgctagtttaggaaaaaaaatcattagtagtgatcttccttatgcccgggatgttttaaaggattatagcggcgtagattttgtaatttacaataatgaagatggctgggctaaggcgttgtttaatgttttaaatggcaattcgaagctcaattttaggccttatgaaaaagatagtcgttcatcttggccacagttcttctctattttgaaataaggtgtattatgtttaatggtaaaatattgttaattactggtggtacggggtctttcggtaatgctgttctaagacgttttcttgacactgatatcaaagaaatacgtattttttcccgggatgaaaaaaaacaagatgacatgaggaaaaaatataataatccgaaacttaagttctatataggtgatgttcgcgactattcgagtatcctcaatgcttctcgaggtgttgattttatttatcatgctgcagctctgaagcaagtaccttcctgcgaattccacccaatggaagctgtaaaaacgaatgttttaggtacggaaaacgtactggaagcggcaatagctaatggagttaggcgaattgtatgtttgagtacagataaagctgtatatcctatcaatgcaatgggtatttccaaagcgatgatggaaaaagtaatggtagcaaaatcgcgcaatgttgactgctctaaaacggttatttgcggtacacgttatggcaatgtaatggcatctcgtggttcagttatcccattatttgtcgatctgattaaatcaggtagaccaatgacgataacagaccctaatatgactcgtttcatgatgactctcgaagacgctgttgatttggttctttacgcatttgaacatggcaataatggtgatatttttgtccaaaaggcacctgcggctaccatcgaaacgttggctattgcactcaaagaattacttaatgtaaaccaacaccctgtaaatataatcggcacccgacacggggaaaaactgtacgaagcgttattgagccgagaggaaatgattgcagcggaggatatgggtgattattatcgtgttccaccagatctccgcgatttgaactatggaaaatatgtggaacatggtgaccgtcgtatctcggaagtggaagattataactctcataatactgataggttagatgttgagggaatgaaaaaattactgctaaaacttccttttatccgggcacttcggtctggtgaagattatgagttggattcataatatgaaaattttagttactggcgctgcagggtttatcggtcgaaatttggtattccggcttaaggaagctggatataacgaactcattacgatagatcgtaactcttctttggcggatttagagcagggacttaagcaggcagattttatttttcaccttgctggggtaaatcgtcccgtgaaggagtgtgaatttgaagagggaaatagtaatctaactcaacagattgttgatatcctgaaaaaaaacaataaaaatactcctatcatgctgagttcttccatccaggctgaatgtgataacgcttatggaaagagtaaagcagctgcggaaaaaatcattcagcagtatggggaaacgacaaacgctaaatattatatttatcgcttgccgaatgtattcggtaagtggtgtcgaccaaattataactcctttatagcaactttctgccatcgcattgcaaatgatgaagctattacaattaatgatccttcagcagttgtaaatctggtgtatatagatgacttttgttctgacatattaaagctattagaaggagcgaacgaaactggttacaggacatttggtccaatttattctgttactgttggtgaagtggcacaattaatttaccggtttaaagaaagtcgccaaacattaatcaccgaagatgtaggtaatggatttacacgtgcattgtactcaacatggttaagttacctgtctcctgaacagtttgcgtatacggttccttcttatagtgatgacagaggggtattctgtgaagtattgaaaacgaaaaacgcgggccagttttcgttctttactgcgcatccaggaattactcggggtggtcattatcatcattccaaaaatgagaaatttattgtcatccgaggaagtgcttgtttcaaatttgaaaatattgtcacgagtgaacgatatgaacttaatgtttcctctgatgattttaaaattgttgaaacagttccgggatggacgcataacattactaataatggctcggatgagctagttgttatgctttgggcaaatgaaatatttaatcgttctgaaccagatactatagcgagagttttatcgtgaaaaaattgaaagtcatgtcggttgttgggactcgtccagaaattattcgactctcgcgtgtccttgcaaaattagatgaatattgtgaccaccttattgttcataccgggcaaaactacgattatgaactgaatgaagtttttttcaaagatttgggtgttcgcaaacctgattattttcttaatgccgcaggtaaaaatgcagcagagactattggacaagttatcattaaagttgatgaggtccttgaacaggaaaaaccagaagccatgttagtacttggcgatactaactcctgtatttcagcaataccagcaaagcgtcgaagaattccgatcttccatatggaggctgggaatcgttgttttgaccaacgcgtaccggaagaaactaacagaaaaatagttgatcataccgctgatatcaatatgacatatagtgatatcgcgcgtgaatatcttctggctgaaggtgtaccagccgatagaattattaaaaccggtagcccaatgtttgaagtactcactcattatatgccgcagattgatggttccgatgtactttctcgcctgaatttaacacctgggaatttctttgtggtaagtgcccacagagaagaaaatgttgatacccctaaacaacttgtgaaactggcgaatatacttaataccgtggctgaaaaatatgatgtcccggtagttgtttctactcatcctcgcactcgtaaccgcatcaacgaaaacggtattcaattccataaaaatatcttgcttcttaagccattaggatttcacgattacaaccatctgcaaaaaaatgcacgtgctgttttatcggatagtgggactattacagaagagtcctccattatgaacttccctgcactcaatatacgagaagcgcacgaacgcccggaaggcttcgaagaaggggcagtaatgatggtcggtcttgaatctgatcgcgttttacaggcattagaaattattgcaacacagcctcgtggagaagtacgcttacttcgtcaggttagtgactatagcatgccaaatgtttcagataaagttctgcgtattatccattcatatactgactacgttaaacgggttgtctggaagcaatactaatgaaacttgcattaatcattgatgattatttgccccatagcacacgcgttggggctaaaatgtttcatgagttaggccttgaattactgagcagaggccatgatgtaactgtaattacgcctgacatctcattacaagcaatttattctattagtatgattgatggtataaaggtttggcgtttcaaaagtggacctttaaaggatgtaggtaaggctaaacgtgccataaatgaaactcttttatcttttcgcgcatggcgcgcatttaagcacctcattcaacatgatacatttgatggtatcgtttattattccccctctattttttggggcgacttggttaaaaaaataaaacaacgatgccagtgcccaagctatctgatcctaagggatatgtttccacagtgggtcattgatgcaggtatgttgaaagccggttcaccaattgaaaaatattttaggtattttgaaaaaaagtcatatcagcaggctggccggataggggtaatgtctgataagaatcttgagatatttcgccagaccaataaaggttatccgtgtgaagttttacgtaattgggcctcaatgactcctgtgtctgccagcgatgattatcattcacttcgtcaaaaatacgatctaaaagataaagtcatttttttctatggcggtaatattgggcatgctcaggatatggcaaacttaatgcgccttgcgcgtaatatgatgcgttatcatgatgctcatttcctgtttatagggcagggtgatgaagttgagctgataaaatctcttgctgcagaatggaatttaactaatttcactcatctaccttcagtgaaccaggaagagtttaaattaattttatctgaagttgatgtcggcctgttctccctttcatctcgccattcttcacataatttccccggaaaattactagggtatatggttcaatcaatcccgatccttgggagtgtgaatggcggcaatgatttaatggatgtaattaataagcacagagccggtttcattcatgttaatggtgaagatgataaactgtttgaatctgcacaattgcttcttagtgattcagttttaagaaaacagctaggtcagaacgctaatgtgttgttaaagtctcaattttcggttgaatcggcggcacatactatcgaagtccgactggaggctggagaatgcgtttagttgatgacaatattctggatgaactttttcgcactgcagcaaattctgaacgtttgcgcgctcattatttattgcacgcatctcatcaggagaaggttcaacgtttacttattgcatttgtacgcgacagctatgttgaaccccattggcatgagttaccgcatcagtgggaaatgtttgtcgtcatgcaagggcaattagaagtttgtttgtatgagcaaaatggtgagatccaaaaacagtttgttgttggagacggtacgggaataagcgtcgtggaattttccccaggagatatacatagtgtcaaatgcctgtcaccaaaagcccttatgttggagataaaggaggggccatttgacccactcaaagctaaggctttttctaagtggttatagggcgatacaccaccgtttattcttctatcttattctatacatgctgggttaccatcttagcttcttcaagccgcgcaaccccgcggtgaccacccctgacaggagtagctagcatttgaccacccctgacaggattagctagcatatgagctcgaggatatctactgtgggtacccgggatccgtgtaggctggagctgcttcgaagttcctatactttctagagaataggaacttcggaataggaactaaggaggatattcatat

SEQ ID NO: 10 - protein, 19 aa, synthetic construct
  REGION 1..19: note = example signal sequence for EPA carrier protein
MKKIWLALAGLVLAFSASA

SEQ ID NO: 11 - DNA, 13043 bp, synthetic construct
  misc_feature 1..13043: note = example O1A rfb locus nucleotide sequence - O1A-EPA production strain stGVXN4411 and stLMTB10217
  source 1..13043: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcgtgaagatacttgttactaggggcgcaggatttattggttctgctgtagttcgtcacattataaataatacgcaggatagtgttgttaatgtcgataaattaacgtacgccggaaacctggaatcacttgctgatgtttctgactctgaacgctatgtttttgaacatgcggatatttgcgatgctgctgcaatggcgcggatttttgctcagcatcagccggatgcagtgatgcacctggctgctgaaagccatgtggatcgttcaattacaggccctgcggcatttattgaaaccaatattgttggtacttatgtccttttggaagcggctcgcaattactggtctgctcttgatggcgacaagaaaaatagcttccgttttcatcatatttctactgacgaagtctatggtgatttgcctcatcctgacgaagtaaataataaagaacaattacccctctttactgagacgacagcttacgcgcctagtagtccttattccgcatcaaaagcatccagcgatcatttagtccgcgcgtggaaacgtacctatggtttaccgactattgtgactaactgttcgaataactacggtccttatcactttccggaaaaattgattccactagtaattcttaatgctctggaaggtaaggcattacctatttatggcaaaggggatcaaattcgtgactggctgtatgttgaagatcatgcgcgtgcgttatataccgtagttactgaaggtcaagcgggtgaaacctataacattggcggacacaacgaaaagaaaaacatcgatgttgtgctgactatttgtgatttgttggacgagatagtcccgaaagagaaatcttatcgtgagcaaattacttatgttgctgatcgcccagggcatgatcgccgttatgcgattgatgctgagaagattggtcgcgaattgggatggaaaccacaggaaacgtttgagagtgggattcgtaaaacggtggaatggtatttggctaatgcaaaatgggttgataatgtgaaaagtggtgcctatcaatcgtggattgaacagaactatgagggccgccagtaatgaatatcctcctttttggcaaaacagggcaggtaggttgggaactacagcgtgctctggcacctctgggtaatttgattgctcttgatgttcactccactgattactgtggtgattttagtaaccctgaaggtgtggctgaaacagtcaaaagaattcgacctgatgttattgttaatgctgcggctcacaccgcagtagataaggctgagtcagaacccgaatttgcacaattactcaatgcgactagcgttgaatcaattgcaaaagcggcaaatgaagttggggcttgggtaattcattactcaactgactacgtattccctggaaatggcgacacgccatggctggagatggatgcaaccgcaccgctaaatgtttacggtgaaaccaagttagctggagaaaaagcattacaagagcattgtgcgaagcacctaattttccgtaccagctgggtctatgcaggtaaaggaaataatttcgccaaaacgatgttgcgtctggcaaaagagcgtgaagaactagccgttattaatgatcagtttggtgcgccaacaggtgctgaactgctggctgattgtacggcacatgccattcgtgtcgcactgaataaaccggatgtcgcaggcttgtaccatttggtagccagtggtaccacaacctggtacgattatgctgcgctggtttttgaagaggcgcgcaatgcaggcattcctcttgcactcaacaagctcaacgcagtaccaacaactgcctatcctacaccagctcgtcgtccacataactctcgccttaatacagaaaaatttcagcagaattttgcgcttgtattgcctgactggcaggttggtgtgaaacgcatgctcaacgaattatttacgactacagcaatttaatagtttttgcatcttgttcgtgatggtggagcaagatgaattaaaaggaatgatgaaatgaaaacgcgtaaaggtattattttagcgggtggttctggtactcgtctttatcctgtgactatggtcgtcagtaaacagctattacctatatatgataaaccgatgatctattatccgctttctacactgatgttagcgggtattcgcgatattctgattattagtacgccacaggatactcctcgttttcaacaactgctgggtgacggtagccagtggggcctgaatcttcagtacaaagtgcaaccgagtccggatggtcttgcgcaggcatttattatcggtgaagagtttattggtggtgatgattgtgctttggtacttggtgataatatcttctacggtcacgacctgcctaagttaatggatgccgctgttaacaaagaaagtggtgcaacggtatttgcctatcacgttaatgatcctgaacgctatggtgtcgttgagtttgataaaaacggtacggcgatcagcctggaagaaaaaccgctacaaccaaaaagtaattatgcggtaaccgggctttatttttatgataacgacgttgtcgaaatggcgaaaaatcttaagccttctgcccgcggtgaactggaaattaccgatattaaccgtatctatatggaacaagggcgtttatctgttgccatgatggggcgtggttatgcgtggttagacacggggacacatcagagcctgattgaggcaagcaactttattgcaacaattgaagagcgtcaggggctgaaagtttcctgcccggaagaaattgcttaccgtaaagggtttgttgatgctgagcaggtgaaagtattagctgaacctctgaaaaaaaatgcttatggtcagtatctgctgaaaatgattaaaggttattaataaaatgaacgtaattaaaacagaaattcctgatgtactgatttttgaaccgaaagtttttggtgatgagcgtggtttcttttttgagagctttaaccagaaggtttttgaggaagctgtaggccgcaaagttgaatttgttcaggataaccattcgaagtctagtaaaggtgttttacgcgggctgcattatcagttggaaccttatgcacaaggaaaattggtgcgttgcgttgtcggtgaagtttttgacgtagctgttgatattcgtaaatcgtcatcgacttttggcaaatgggttggggtgaatttatctgctgagaataagcggcaattgtggattcctgagggatttgcacatggttttttagtgctgagtgagacggcggagtttttgtataagacgacaaattattatcatcctcagagtgatagaggaataaaatgggatgatccaagcatcaatatttcatggccagtcgattcacaagtgctgctatcagctaaagataataagcatcctccattaacaaagattgaaatgtatagttaagatcacgataaatcttggaagggttgcaaaattgaataaaatagtgagcaaaagtgaaataaggaacgtaatccacaatgctggctatatgatgattactcagatagctttatatgttgcaccattatttatactgagttatctgttaaaaacactgggggttgcacagtttggtaattatgccttaatactatcaatcgttgcatatttacagattataacggattatggtttttcttttagtgcaagtcgtgcgatctcacagaatagagaggacaaagaatatatatcaaaaatttatctgtcaactatgactatcaagttggcgatatgcgctttcttattcttattgctcatgctatttttaaatcttttgcctgtgcaagctgaattaaaacaaggaatattatatggatatcttcttgtaataggaaatactttccaaccacaatggtttttccaaggtatcgaaaaattaaaaatcatagccctttctaatgttatatcaagatgcgccgcgtgtttacttgtatttatctatgtgaggaatagcgaggatttacaaaaagcacttttagtacagtcacttccattagtaatttctgcgattggattaaatatatttatattgaaatatatcaatattatttttccggaaaaaaaattatttaaggtaattttaaaagaaggtaaggatttttttcttgcatcactttattctgttattctcaataatagtggcatttttctattagggatttttactaatcctgttattgttggtgtatatgccgccgctgaaaagatagtcaaggccgtattgtcgctatttacaccactgacgcaagctatatatccttataattgtcgtaagttttcactatccgtatttgacggcattgaggcagcaaaaaaaactggtataccaattataattttagcatttatagctgctgttatcgttgcaattaccttacctgttgcaatcgactatcttaattttccaaaagaaacaatttttgtaggtcaaatattaagtgcatggatcttttttggtgttcttaataatgtattcggcattcagatattgagtgcatcaggaagaagtaaaatatatagtaggatggtattcgtatcagcgcttataacattacttttgattactctattattgcagttttgtaacgccactggagtggcatgtgcaatattattgggtgaaatgttcttatcaatattgttacttaagcgatataaaaaaataatttaaggaatagttatgaagaagttattattagtgttcggtactaggcctgaagcaataaagatggcctctatcattgaattattaaaaaaagattgtagattcgaatataaaatatgtgtgacaggccaacataaagagatgcttgatcaagttatgcaagtatttgatgttaaacctgattataatttacggattatgcagcctgggcaaacattagtatctatagcaacaaatatactctcacggttaagtgaagttttaattatagaaaagccagatattatacttgtgcatggggatacaacgactacccttgctgctactttagctgggtattaccaccaaataaaagtttgtcatgtggaagcaggattaagaacaggggatatttactctccttggcctgaagagggcaatcgtaaagttacaggggcattagcatgtattcatttcgccccaacagagagatcaaaagataatctcctgagggagggggtcaaagtaaataatatatttgtaacgggtaataccgtcatcgactctttatttattgcaaaagatatcatagataatgaccctaatataaagaacgctttacataataaatttaattttcttgataaaagccgacgagtagtacttataacaggtcatcgaagagaaaatttcgggaaaggttttgaagatatatgctttgcaataaaggaattagctttcatttatcctaatgtagattttatttatccggtgcatcttaatcccaatgtaatggaaccagtacatcgtatattagataatatatgtaatatttaccttattgagcccttggattatttgccttttgtttatttaatgaatgagtcatatttaatattgactgattcaggggggatacaagaagaagcgccttcgttaggtaaaccggttttggttatgcgtgatactactgaacgccctgaggcggttgaggctggtactgttgtattagtggggacttctaagataaaaatagtaaataaagtaacggagctattaaacaatgctgatatctacaatgctatgtctctgttacataatccatatggcgatggaacagctgctcaaaaaattcttaatgtgctcgcccaagagctaatttaatttaagctaaaaatatgttattaattattgctgattatccaaacgaaatgaatatgcgcgagggagctatgcaacgaatagatgcgatagactctctcattcgagatcgcaagcgagtgtatttgaatatttcattcaaaaagcatctagttcgctcaaatagttcctttaataatgttatagttgaaaatctaaatgcaattattcacagaaacatcataaaacagtacatgcaaaaatcaacaactatatatgttcattctgtttataatttattaaaggttataacgctcattgatctaaaaaaaacaattcttgatatacatggtgttgtaccggaagaacttttggcagataataaaaaattacttagtaaagtatataacatggtggaaaaaaaaggtgtccttggatgcaaaaaattaatacacgtcagtacagaaatgcaaaaacactatgaagcaaaatatggagtaaacttggctgaaaggtcaatagtgctcccgatttttgaatataaaaatataacccaatcgcaaaacaaatggacagaaaataaaatacgaagtatctatcttggaggattacaaacatggcaaaatattgataaaatgattcaagtttgtgatgacacagtgataaacaatgaagcaggtaagtatgaattcaactttttcatcccacagagtaacttggaagggtttatagataaatattcgttaaaattacataatatcaatgctaatgcatctacgctatcacgtgatgaagtaattccctttctaaaagaatgtcatattggttttgtattgcgcgatgatataatagtaaacagagttgcgtgccctacaaaattggttgaatatttagagtgtggtgtcgttccagttgtgctctccccacttataggtgatttttattcgatgggatatcaatacattactacagaggaaatggctaacagaagtataagtttgttggatcttgaaaaaatggctgcacataatttacaaattttgacttcttatcagaagagaacctacaaggcacagaaagaacttattgctcaactgtgctgaattttttacatatataaaattatgtaagcatatcgcgggtcaggtaattgtatgcgtatcaaatataaagataacggttatatattatgttttctattatgtttcattttgagctacttagttttactcaaatctgactactttcctgctgattttctgccatatacagaaatatacgatgggacatacggagaaatcaataatattgagcctgcctttttatatttaacacggttgtttcattatttaaatttcccctatatattttttgcaatgttagtttgtgccttatgtttaagttggaaaataaaatatgcaagaaaaataattaaagatagttatatatatttgttcttgtatgtatatgtatcattttatgtgtttttgcatgaaatgactcaattgcgcatagcaattgcagtcactatgtgctatgtgtcggtttattattacttttataaaaattgtattaaacatgcactgccatggatggtgttggctattttgtttcattacagcgccttgcttttatttatgtcattatttatatacagttataggaggttattaatagtaattatagggtttgtaatatgtatgagctttttaaacgtgtatgcagatacaattgcactatatttgccaaatgaaaaaatagtaaattatttatatagtatttcatcatcattagacaatagaaatgatttggcaatattcaacctgaataatataatatttttatcaatatttattttgatcttttatcttagccgatatataaaattaaatgataatgaggcgaagtttattaagtatgtgcaatgttcaggaatattagccttttgtattttctttctggctagtggagtcccggtcattgcttatcgaactgcagagttgctgcgaatattttatccgatggctttagtattaatcctttcgcatataaaaaataataatatgcgttattttattgcagtcattatagttatcctttcaggcttaatgttgtttataacactaagggctgtatcaatagttggtcaaggattataaaatgaatgttgctattttgttgtctacgtataatggcgaaaaatatttagaggaacaactggattcattgctgcttcaaagttatcaggattttgtagtgtatatccgtgatgacggatcatctgatagaactgtaaatataataaaccaatacgtaatgaaagataacagatttattaacgtgggtaattcagaaaatcttggttgtgctgcttcgtttattaatttattaagaaatgcttcagccgatatttatatgttttgtgaccaagatgattattggcttccgaataaattacagcgtgctgtggattatttttcggctattgatcctttacaacctaccttgtatcattgcgatctaagcgttgttgatgaaaaacttaatattatacaaaattcatttttgcagcatcagaaaatgtcagcgtatgattcaatgagaaaaaataatcttttcatacaaaattttgttgttggttgttcatgtgctgttaatgcttcacttgcggaatttgttctttcgcgaattggagagcagcatgtaaaaatgatagctatgcatgactggtggttagccgtgactgcaaaactttttggtcgaatccattttgataatactcaaacgattctttatcgacaacatcagggcaatgtattaggtgcaaaatcatcaggtatgatgcgttttattcgattaggattaaatgggcaagggatttcgcgagtagtatcttttagaaaaaaagtttgtgcgcaaaataagcttcttttagatgtctatgataaagatttaaatcttgagcaaaaaaaatctatcaggcttgtaattgagggccttaaagagaactcttcaattgctgaccttttaaaatgtttctatcatggtagctatatgcaaggttttaaacgtaatcttgccttaatatattcagttctttacacaaaaaaaagaagatagtgtatccttatgaaaaaaattgctattatcggtactgttggcataccagcatcatatggcggatttgaaacattagttgaaaatttaacaagatacaattcctcgggagttgaatataatgttttttgttcatcgtttcactacaaatcccaccaaaaaaaacataatggggcccgtttaatttatattccgcttaaagccaatggatggcagagcattgcgtatgacataatttcgttagcatattctatttttttgaagcctgatgtgattctgattttaggggtttctggttgttcatttttgcctttcttcaaactcttaacacgcgctaagtttattactaatattgatggcctggaatggcgaagagataaatggaattcaaaagtgaaacgtttcttaaaattttcagaaaaaatcgcagttcaatattcggatgtcgttattacggataatgaggcaatttctgagtacgtttttaacgagtataataaagatagccgagttattgcctatggaggggatcatgcatggttaaatactgaggatgtatttacaacaagaaattataaaagcgattactacctttctgtatgtcgtatcgaacccgaaaacaatgtagaattaattttaaaaacattttcaaagctaaaatataaaataaaatttattggaaattggaatggcagcgagtttggaaagaaacttaggctgcattattctaactatccaaatattgaaatgattgatccgatttatgatcttcaacaattatttcacttacgaaataattgcataggatatatacatggtcattcggctggaggaacaaacccttctttagtcgaggcaatgcattttagtaaacctatatttgcatatgattgtaagtttaataggtacactactgaaaatgaagcatgttatttttctaatgaatctgacctcgcagagaaaatcataatgcattgtgagctatcattaggtgtctctggcacgaaaatgaaagaaattgctaaccagaaatacacttggagacgaatagcagaaatgtatgaggattgctattaactctgttaaacttcaaatcttttacaatatatggcatgactataagcgcattaattgtttttcaagccgctctcgcggtgaccaccccctgacaggggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtatcgataaagaaggtgtgttccataccgaatggctggattaa

SEQ ID NO: 12 - DNA, 13790 bp, synthetic construct
  misc_feature 1..13790: note = example O2 rfb locus nucleotide sequence - O2-EPA production strain stGVXN4906
  source 1..13790: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagtgaaaatacttgttactggtggcgcaggatttattggttcagctgtagttcgtcacattataaataatacgcaggatagtgttgttaatgtcgataaattaacgtacgccggaaaccgggaatcacttgctgatgtttctgattctgaacgctatgtttttgaacatgcggatatttgcgatgcacctgcaatggcacggatttttgctcagcatcagccggatgcagtgatgcacctggctgctgaaagccatgttgaccgttcaattacaggccctgcggcatttattgaaaccaatattgttggtacttatgtccttttggaagccgctcgcaattactggtctgctcttgatagcgacaagaaaaatagcttccgttttcatcatatttctactgacgaagtctatggtgatttgcctcatccagatgaagtaaataatacagaagaattacccttatttactgagacgacagcttacgcgccaagcagcccttattccgcatccaaagcatccagcgatcatttagtccgcgcatggaaacgtacgtatggtttaccgaccattgtgactaattgctcgaacaactatggtccgtatcacttcccggaaaagcttattccattggttattcttaatgcactggaaggtaaggcattacctatttatggcaaaggggatcaaattcgcgactggttgtatgtagaggatcatgctcgtgcgttatataccgtcgtaaccgaaggtaaagcgggtgaaacttataacattggcggacacaacgaaaagaaaaacatcgatgttgtgctgactatttgtgatttgttggatgagattgtaccgaaagagaaatcttatcgtgagcaaattacttatgttgctgatcgcccagggcatgatcgccgttatgcaattgatgccgataaaattagccgcgaattgggctggaaaccacaggaaacgtttgagagcgggattcgcaaaacggtggaatggtatctggctaatacaaattgggttgagaatgtgaaaagcggtgcttatcagtcatggatcgaacaaaactatgagggccgtcagtaatgaatatcctgcttttcggcaaaacagggcaggtgggttgggaactgcagcgtgctctggcgccgctgggtaatctgatcgctcttgatgttcactccactaattattgtggagatttcagcaaccccgaaggtgtggcagaaaccgtcaaaaaaattcgtcctgacgttattgttaatgctgctgctcacactgcagtagataaagcagaatcagaaccggatttcgcacaattacttaacgcgacaagcgtcgaagcgattgcaaaagctgctaatgaagtcggggcctgggttatacactactctactgattatgttttcccaggcagtggtgacgcgccatggctggaaacggatgcaacagcaccgctaaatgtttacggtgaaacaaaattagctggggaaaaggcattacaagaacattgcgcaaagcatcttattttccgtaccagctgggtatacgctggtaaaggaaataactttgctaaaacgatgttgcgtttggcaaaagaacgcgaagaactggctgtgataaacgatcagtttggcgcaccaacaggtgctgaattgctggctgattgcaccgctcatgccattcgcgtggcattaaaaaaaccagaagtcgctggcttgtaccatctggtagcaagtggcacaacaacctggcacgattatgctgcgctggtttttgaagaggcgcgcaaagcagggattaatcttgcacttaacaaacttaacgccgtgccaacaacggcctatcccacaccagcccgtcgaccccataactctcgcctcaatacagaaaagtttcagcagaactttgcgcttgtcttgcctgactggcaggtgggcgtgaaacgtatgctcaacgaattatttacgactacggcaatttaacaaatttttgcatctcgctcatgatgccagagcgggatgaattaaaaggaatggtgaaatgaaaacgcgtaaaggtattattctggctggtggttccggcactcgtctttatcctgtgacgatggcagtgagtaaacaattgctgccgatttatgataagccgatgatttattatccgctttcaacgcttatgttagcgggtattcgcgatattcttattattagtacgccacaggatacaccgcgtttccaacaattattgggggacgggagccagtggggtcttaatctacagtataaagtacaaccgagtccggatggcctggcgcaagcgtttattattggcgaagactttattggtggtgatgattgtgcactcgtacttggcgataatatcttctatggacacgacttgccgaaattgatggaagctgctgttaacaaagaaagcggtgcaacggtatttgcttatcacgttaatgatcctgaacgctatggtgtcgtggagtttgataataacggtacggcaattagcctggaagaaaaaccgctggagccaaaaagcaactatgcggttactgggctttatttctatgacaatgacgttgtggaaatggctaaaaaccttaagccttctgcccgtggcgaactggaaattaccgatattaaccgtatttatatggaacaaggacgtttgtctgtagccatgatggggcgtggctatgcatggttggatacagggacgcatcaaagccttattgaagcaagtaacttcattgcaacaattgaagagcgtcagggattaaaggtatcttgcccggaagagattgcttaccgtaaagggtttattgatgccgagcaggtgaaagtattagccgaaccgcttatcaagaatcaatatggtcaatatttgctgaaaatgatcagcgaatagtatatgggaactcaatgatggatattaaattaatctctttgcaaaaacatggggatgagcgcggtgcattaattgctcttgaagagcaacgaaatatacctttcgaagtcaaaagaatatattacatacttgagactcttaatggagtaagacgcggatttcatgcgcacaaggttactcgtcagttagctattgtagtcaagggagcttgtaaatttcatctggataatggtaaagaaacaaagcaggtggaacttaatgatccaacaattgcgttgctgatagaaccctatatatggcatgaaatgtatgattttagtgatgattgtgtgctgcttgtaattgcggatgatttctataaagagtctgattatatccgcaattatgatgattttattagaagagtaaattcaattgagaattcataagctaagtgacgtccagacaacatcaattggtgatggaacaactatctggcagtttgttgtgatactaaaaggtgctgtaattggtaataattgcaacatctgtgcaaataccttaattgaaaataacgttgtaattggtaacaatgtcacagtcaaaagcggtgtgtatatttgggatggcgttaaaatagaggataatgtttttattggtccttgtgtagcatttacaaatgataagtatcctcgctctaaagtctatcctgatgaatttttgcaaacaataatacgcaaaggagcatcaataggtgctaacgcaaccatcctgccaggaattgaaattggtgaaaaagcaatcgttggtgcggggagtgttgtaaccaaaaatgtaccgccatgcgcaatagtagtaggtaatccagctcgatttattaaatgggtagaggataatgaataaaattgattttttagatctttttgcaattaaccagcgacagcacaaagaattagtctctgcgtttagtagggtgctagattctggttggtatatcatgggcgaagaacttgagcagttcgagaaagagttcgcagaatactgtggagttaagtattgcattggtgtagcaaatggccttgatgcgttgatactagtattgagggcatggaaagaacttggctatcttgaagacggtgacgaggtattagtaccggcaaatacatatattgcttctattcttgctataacagagaacaaacttgttcctgttcttgttgaaccagatatagaaacttataatattaatcctgctttaattgaaaattacattacggaaaaaactaaagcaatattaccggttcacttatatggtctattgtgcaatatgccagaaattagtgcaatcgccagaaaatataatctgttgattcttgaagattgtgcacaagcacatggtgcaatacgtgatggtcgcaaagctggagcttggggggatgctgcaggatttagtttttatccaggaaaaaaccttggagctttgggggatgcgggagctgttactacaaataatgcagaattatcctcaactataaaagctttgcgaaattatgggtcacataagaaatatgaaaatatttatcagggattgaatagtcgattggatgaactgcaagcagccttattgcgtgtaaaaatccatacattaccggaagatactgcgattcggcaaaggattgctgaaaaatatattcgtgaaataaaaaaccctgcgattacgttaccagtgtacgaaggccaaggtgcgcatgtttggcatttatttgtagtaagaatcgctaatcgtgaaaaattccagtcatacttattagagaagggtatcaaaaccttaattcactatccattaccaccccataagcagcaagcatatcaaaatatgtctagccttagccttccaattactgagcaaattcatgatgaagtcatttctttacctataagtccggtaatgagtgaagatgatgtcaattatgtaatcaaaatggtcaatgattacaagtaatgaaaaaatttcttcaggtaactatattatccgctatctatacattcattaaaatgattgcgggttttatcatcggtaaggtagtagcaatttatacagggccatcaggggtagcaatgcttggccaagtgcaaagtttaatcacaatagttgcaggtactacctctgcacctgtaagcacaggccttgttcgatatactgcggaaaattggcaagaaggacaagaagcatgcgcgccatggtggcgcgcatgcttaagggttactctgtttttattcttgcttattattcccgttgttattatattgtcgaaaaatattagtgagttactttttagcgatggacaatacacatggttaatcattttcgcatgttgtatattgccattctccattataaatacattgatcgcttcagttttaaatggtcaacaattttataagcaatatatattggttgggatgttttctgtattcatttctactatgtttatgattttgttgattgtagcttataatcttaaaggtgcattgattgccacagctataaatagtgctattgctggtcttgtattggttttattttgtctcaataaatcttggtttagatttaaatattggtggggtaaaacggataaagacaaaattataaaaattattcattatactctgatggctctggtttctgttatctccatgcctacagcattgatgtgtattagaaaaatattgattgctaaaactggttgggaggatgcagggcaatggcaggccgtatggaagatatctgaggtttatcttggtgttgtgacaattgctttgtcaacatatttcttaccaagattgacaattataaaaacaagtttccttataaaaaaagaagtaaatagtactatattatacataatatctattacttcattcatggcgttgagtatctatttattccgcgatttggtaataacagttttatttactgaacagtttcgctcagctcgtgaattatttttattacaacttataggggatgtaataaaaattgctgggtttctttatgcataccctcttcaaagtcaggggcatactaaactattcatcagttcagaagtgattttttctatgctctttatcattaccacctatatttttgttgtaaattatggagtacatggtgctaacataagttatgtcattacatatagtttatattttgtgtttgcatttgtgtttactaattttattaatgttagaagaaataattaaaaacagaggttgaattttgaaaataattatacctgtcttaggatttggcagggctggtggtgaaagagttctttctaagctggcaactgaattgatgaattatggacatgatgtaagttttgttgttccagataatagaactaatccatattatgctaccacagcaaaaattgtcacgagtaaatctagtcaaaaccgtgtaaaaatattgagaatcattaaaaattactataatctgtggcgtaaatgcatagagttaaatcctgatgctgtagttgctagttttcatttgactgcctatcttgtcgcattattaccaatcacccgtcgtaagaaatattattatattcaggcgtatgaagttaatttttttgataatataatatggaaattaatagcgggtttaacatattatttaccgcttaaaaaaatactaaatagtcctaatttgcttcctcataaacatgatgattttataggagtagttcctgcaggagtagatttaaacgttttctatccgaaaccatcaaataggttattaaatggtcacacatcaatagggattattggtagaaaagagaagcacaaaggaactagcgaaattatttcagtattgtgttcactggaaaataaagctggaattataatcaatattgcgatctatcttgaagaagttgataagcagcgtttaatcgctgccgggtttcaggttaatttttttccgattacttctgatttagaattggcatccttttatcgaagcaatgacatcatgattgctgttgggttaattgaagatggcgctttccattatccttgtgctgaatcaatggcttgtggttgtcttgttatttcaaattatgcgccacttactgaaactaacagtgtacttaaattagtcaagtttgatgcttgcaaacttggtgaagcaattaatctttgtctcaatcttgacctagaagaaaaaagcaaagaaatccaatctaatatttctgtgttgaataaatatgactggaaaattgttggtgaaactttcaatagtttattgttagatgcaaataaatagtatacgttgatggggaaaatatgaatattgttaaaactgatattccagatctgatcgttcttgaaccaaaagtgtttagtgatgaacgcggcttttttatggagagttataatcagattgaatttgagaaggcaataggaaggcacgtaaattttgttcaggataatcattcaaaatctagtaaaggcgtactacgtgggttgcattatcaattagcaccgtatgcacaggctaaattagttcgatgtgttgtaggtcaggtatttgatgttgctgttgatcttagaaaaaattcaccaacgttcaaaaaatggtttggaataaccctttccgcagaaaataaacgacaattatggatacccgaaggatttgctcatggtttcttggtgaccagtgatgaagctgagttcatttataagacaactaactactatgctcctggtcatcagcaagcaattatttacaatgatcctattttaaacatcgattggcctttctgcagtagtgctctgtcattatcacaaaaagatcaagaagcaaaattattttcagaattattggacagtgaactgttctaataaagtgtgccaccttatccgtctgaaggataggtggttgcttatatttttttgagtatgtttgtataatgacagaaaatagtccgaaatataaacacgataaaagcttaataagttttatctacttattttttatatttacacttattgtaggctttattatcgcaaatacccagtttttggggcgaagtagagactatgataattatatacagatcttttctggtaaagaaggggagggggttcttgaattattttatcgcggattgatgttaataacgaccagctatgaaactatcatttttataattttaacatgttctttttttataaaggcaaggtttctcgctaactattcgcgtaatttttcaggcttgaccttattctttatttattatgcaagcgttgcactttgggttttagattatactcaattcagaaatggtctatgtatttccattttaatgttttccgtatactatttatttataaataaaccgacttatttttatttctcggtattatgtgcaattgcaactcattggtctgctttgccttttttgcttttatatccttttgtctattcaacaaaaataagacgccttggttatttttgtttcagtattcttgttttgattgcgatctcaggagaaggaaaagagatcatatcttttataagaaattttggagtgggacaaaaaataggaaatgaagctggtgtaaatttaataaattcattatcccttaccgctatttcctggtttattattagttacatatcaagcattggaaatgaaaggagaaatttaaggcttttcttttgttatggtgtcatgcaatacgtgacttttagccttttctctctacctgttatggctttccgtattttggaaatgtattttttccttatgctaaccattggggtgtttattaagcaaaaaaagaattattattttattttttgcaaagtgttaattttattgtatctaacatactattatcatatggtctttggagtgattaatgtgtaaggctaaggtgttggctataattgttacttacaacccggaaattattcgattgacggaatgtattaactctttagccccacaagttgagagaataattcttgtagataatggctcaaataatagtgatttgataaaaaatatcagtattaataaccttgaaattattttactttcggaaaacaaaggcattgcatttgctcagaaccatggtgttaagaagggcctggaagcaaaagagtttgactatttatttttctcagatcaggatacttgctttcctagcgatgttattgaaaaacttaagagtacatttacgaaaaataataaaaaaggtaaaaatgttgcttgtgcttctcctttttttaaagaccatcgttcaaattatatgcatccgtcagtcagcctaaatatttttacgagtacaaaagttatatgtagtgaagtagacgatgatctttatccctcgcatgttattgcttctgggatgttaatgtctcgtgaagcatggcgcgtcgtcggaccattttgtgaaaaactctttatagactgggttgatacagaatggtgttggcgtgcattagctaataatatgattattgttcagacaccatcagtcatcatttctcatgaacttgggtatgggcagaaaatttttgctggtcgatctgttacaatacataattctttcagaaatttttataaaatacgcaatgcaatatacttaatgctgcattcaaattatagcttcaagtatcgttatcatgctttttttcatgcgacaaagaatgttgtatttgaaattttatattcgaaagaaaaattaaattcactgaaggtttgttttaaagctgtacgtgatggtatgttcaataatttttaatacgaaaatagttaggctcaaggtgtttaaatggaagaaaataatatgaagacggtcgctgtagttggcacagtgggtgttcctgcttgttatggtgggttcgaatcacttgttcagaatctaattgattatcaatctgatggtatacaatatcagatattttgctcttcaaaaaaatatgataaaaaatttaaaaattataaaaatgcagaattaatctatttgccgataaatgccaatggcgtctctagcataatttatgatattatgtgtttaattatttgtttattcaaaaggccagatgttgttttaatattgggggtgtctggttgtttatttctaccaatttataaactattttcaaaatcaaagattattgtcaatattgatgggcttgaatggcgtagaaataaatggggaacgtttgctaagaaatttcttaaaatatctgaggcgatatctattagaatagctgatattatcatttcagataatcaagcaatagctgattatgtggaaaataagtacaagaaaaaaagtgtagttatagcttatggcggagatcatgccactaatcttagtacaccgatagacaatgatcaaaaaaaagaaggttattatttggggctttgtaggatagagcctgagaataatatagaaatgattctgaatgccttcattaatacagataaaaaaattaaatttatgggtaattgggataacagcgagtatggacgccagctaaaaaaatattattcaaactatccaaatatcaccctactagaacctaactataatattgaagagctttataaactaagaaaaaattgtcttgcatacattcatggacactcggctggtggaacaaacccttctttagttgaagcgatgcattttaatattcctatttttgctttcgattgtgactttaatcgttacacaactaacaatttagctcattactttaatgattctgaacaacttagcttattagcagaaagtttgtcttttggaaatcttaaatgtcgagtattagatttaaaaaattatgctgaagatatgtataactggaggcatatagctgctatgtatgaatctatttattaaacgcattaacaataatataattgaccttatatagcagggaaagatcacgtaacgctgcggcgcgccgatccccatatgaatatcctccttagttcctattccgaagttcctattctttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtatcgataaagaaggtgtgttccataccgaatggctggattaa

SEQ ID NO: 13 - DNA, 13777 bp, synthetic construct
  misc_feature 1..13777: note = example O6A rfb locus nucleotide sequence - O6A-EPA production strain stGVXN4112 and stLMTB10923
  source 1..13777: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatttgcccgccgggcgtgacaattatgaacgtgcgtcagggcgaacctttaggtttgggccactccattttatgtgcacgacctgccattggtgacaatccatttgtcgtggtgctgccagacgttgtgatcgacgacgccagcgccgacccgctgcgctacaaccttgctgccatgattgcgcgcttcaacgaaacgggccgcagccaggtgctggcaaaacgtatgccgggtgacctctctgaatactctgtcatccagaccaaagagccgctggaccgcgaaggtaaagtcagccgcattgttgaattcatcgaaaaaccggatcagccgcagacgctggactcagacatcatggccgttggtcgctatgtgctttctgccgatatttggccggaacttgaacgcactcagcctggtgcatgggggcgtattcagctgactgatgccattgccgaactggcgaaaaaacagtccgttgatgccatgctgatgaccggcgacagctacgactgcggtaaaaaaatgggttatatgcaagcgttcgtgaagtatggactacgcaacctcaaagaaggggcgaagttccgtaaagggattgagaagctgttaagcgaataatgaaaatctgaccggatgtaacggttgataagaaaattataacggcagtgaagattagcggcgaaagtaatttgttgcgaattttcctgccgttgttttatataaacaatcagaataacaacgacttagcaataggattttcgtcaaagttttccaggattttccttgtttccagagcggattggtaagacaattagcatttgaattttacgggtttagcgcgagtgggtaacgctcgtcacatcgtagacatgcatgcagtgctctggtagctgtaaagccaggggcggtagcgtgctgaaattataaagtcattcttatagaacatcgcatttcaataatataattacacctaaatgaataggatacaacgtgtgcacaattatttaaggcttaaagataaaataaaaaacgtatttttagggttgtatatattgcagttatttaattatatcgcgccattggtaattatccctatcctgataaaatatattgggttgggggaatatggggaattggtctatattacatctatttatcaaatagtggctttgattattgattttggctttacttacacaggacctgtggttgctgcgagacatagatgtgagacccaaaatttacagcgctattactcaatagttgttcttttaaaatcattgctttttataattgcattaacatgtgtatttttattgtgcagattaaatatagtccacttgtcattttttgggtttttgtcaatttttctatgcactattggtaatatattatcgcccaattggtttttgcaggggattggtgattttaaaaaactttcatactcacaagtaatagtgagaataacattgtttatcatacttcttgtttatgtctgtagtggcggagataatgtttttatcctaagttttttgcaaaatgcaacattactcatatgctgtatatacttatggccaaatattcatattagccatgttgttcatcttaaacctaatgaatgcattgtggaatttaagaaggcaggaaatgtttttattggcgtaataggtacgattggttacaatggtctaattcctgtgttaattggaaacctttgcggtaatacgagtcttggtgttttttcaatcgttcaaaaaatgacaacagcatgtcaaagtctaattaatccaatatcacagtatatgttatctcaagtttcagaaattaaacctcaagataaactgttttattatagaattaaaaaaagtttttttgtgcatttaacaattagcataattgcatgtttatgttatatggggttagggcaatatgtggcgacttttataggtaaagttgacgtttcatttgttattattttatttgcgtcaataattaccattttttcatctttaaataatgtccttggtatacagtttcttataccgacagataatgtaaaaatactacgaagtataaatgttatggcgggaattattgttgttagtttgtcctggctgttaatatcacgctttgacattctggggggggttttattaaacctaattggtgagtttcttgtattcagtatgctagcttttattgcccatcgaaagtggggagcgagagtataatgaaagtgaaggcggttcctgctattacattctatttaagtttaatgctgacaattttagtgttactgtttggtaatgaaccaaataaatcacaatatatccttgttatagcaacgataacagttttttatatcgcatatatcactaataaaataacttctccggccagccttctcgttatatcatcttttgtgtttttaggttgtcgccctttattatctttgtttgcaaactatgattataggattgccgattggtttattgaaggatatatggatgacgatgtgattttggctaactatgctataacactaatgtattatggttatacattgggactaattctatgcaaaaatactgaaaaattttatccgcatggtccttatcctgaaaaacaattgctaaaaataaagtttcttttgactttattttttctgggttcgataggtatggttgtaaaagggatattcttttttaactttatagaatctaatagttatgttgatatttatcaatcaaatataacaacgccaataggttatgattttctatcttatttattttattgttcttttttccttatatgtgcgtttcatatacagttcagaacaaataaaaaatttctttttattgcgatatgcattgctgcatttagcaccttgaagggtagtcgtagtgaagctataacgtttcttttaacggttacatgtatatattttaatgaagtaaagacaagaaacttacgtctgctgattacaatgatttttgtttttagcgtcatttttgtgattagtgaatttatctcaatgtggcgcactggagggagtttttttcaattaatgcagggtaataatcctgttataaactttgtatacggcatgggagtatcatatctttccatttatcaatcagtaaaactacaactattgtcagggggatataatgttacctatctattcagccagttaataataacttgctcgtcaatatttaatgtcaaattgagcttgccggaaataagctatagccatttggcctcatacacagcaaacccagaactatataatcttgggttcggacttggggggagttatttagcagaatcgtttttagcatttggtctgattggatgtttcattataccctttttacttttacttaatttaaatgtattggaaaaatatacaaaaaacaaaccaattatatattttgtttattatagtgtgttgccacctatattattcacaccaagagagactttgttctatttcttcccctatcttgtcaaaagtatatttgttgcttttttagttacattatacatccagtataaaaaggattgaccaaaatgtcagaaaaaaatgtcagcataataatcccaagttataacagggctcatattcttaaggaggtcataccaagttattttcaggatgagactttagaggttatagttatcaatgatggatcaacagataatacaaatagtgtattagctgaactgaaggaaaaatattctcagttagttattttagaaaatgaaacgaataaaaaacagatgtattctaaaaaccgagggattgaaatagccaaagggaaatatattttttttggtgatgatgactcttacctcttacccggtgttatatctcggttattggctacaaaatatgagacaggcgctgatgtaatcggcgcaagaatactttatatgaataataacgagaaaacaattgaagattgcataaatcgacataaaaaagaggggcgttttgttagtgatctaaatagattggattttagttatacatgtgatttggaccatccgattgaatgtttttatgcacagccttttgttctagctgaaagggaactaatatcgaaatatcgatttgatatatcttatacgggaaactgctatcgtgaggaaactgatttcatgctatctctatttattaaaaataaaaaatttatatatgattcaaaggctttgttaataaatttacctccaagaaaagcgacgggaggggcaagaacagctaatcgattaaaatatcattacgaaagttgcataaataattatagatttttaaaaaaatataatgataatttgaatcttctttcaggacaaaagcatgctatattttaccgacagtgtcaattcgttctgctaaaaatgaagtcgtttatcgggaagtttttaaaatgattatatatatcgccgcgtataatggttcaggagggcaaggtggggtggaaagggttgttgcccaacaatgtaacattcttaaaaatttgggggttaaagtcattatacttgataaaacatacttcaaaatttctaacaaaattcgtaacaaaaaaatacaagtagcactttatccaatattagtttctctttatttaaccttacaaaaattacgtggcgtgacgtttaaagttattgcacatggctattgttctcctttttataggaatgacatcttaatagctcatggcaatatgaaatgttattttcaaacagtcatgaataaaaaacctaatcggttgtctggcagtggtcttttatctttctatgagcgttgggctggagcattttcaaaaaatatctgggctgtttcaaataaggttaaaagtgaatggaatgagctttacaatattaattcacataaaatcaaagttgttcgaaattttataaatcttgcacaatttgattacactgatgttaatgaagcagaatatgtgacatttgtcgggcgattggaaaaaggaaaaggaatagatgatctgtattacatatgtaaaaatctgccagatacttccttccatttagtttcaagtattcccgccccacaaaattttgcttcgctaaataatgttctgaccagcattgctgtcccctatgcgaaaatgccagaaatatttaagaaatccagagtacttattttaccgtcctattatgaaggatatgagctggttactattgaagcgctatgctgtggttgccctgtgataggctataatgttggtgcaattagagagttgtatgcagaaagttttcctggcgtatttattgccaataataaagaagatttagcacaagtagcctacaaattaattagtcttgataatgaaaaatattatcatttgagacaaactatttatagcaagcgtgagcttttttctgaagagagatatgcggaaattttaacggcggcatttaatgaaaaaaaataagaaactctgtctcatttcaattaactcatataatgaacttaccggaggaggagtatatttacgtacgcttgttagttttctacaaaaacagaatgttaatttaacacttattgataaaaaatcctcaggtaaactattcgaagacaatacttttcaacatatatcatttattaaaggtaaacgtcaggatataatatccaggcttttttttataccatcattttatgtcccttatattttctcaataattaaaattttacggaagcaagatattcttgcttttcacaactctcggcttggattgttatgtctgctttttagaatactcatgccccacaaaaagatcatattgtttacggataacttcgaatatgacttaataagacaaaaagataaaaacataactacttttattgaaaaattaattgtttatctcaatgaatttatcgggcttaagaattcagatttagttagctatattacccggcaagataaaaatgcaatggataaattttatgggattaaaaaaagcagaaatttaattctccctgtgatatttagtagagaaaaaccaactgatgtattgtcagctcactttattaatgagtataatcgattgaataatgataataggaaaaaagtagtatttactgcatcttttgatttttttccaaatatagatgctgccaactatgttttaaatgcagcaaagtctaataatgattattgctatattttggcaggtaggaaaagtactactttgaatcttcctgatttggataatttattttttttcgataatctatctaatagtgaaatgtcatatttattatctgcttgtgatgttttttattctcctatagttttaggaagtggaatgaaaacaaaaattgcagaagcactatcatatggattatatatttatgcgacagagcattccttaatcggctatgatgaaattatacacaataaggagtgtgttaaaaaaatctcacatttggatgaggaatttcctaaagatttcaagatgaaaagtatcaataaacagctaataatgtcttatcagcaaaaatattattcacattatcggtttaatggccatgaacttgatataataaattttgacgattagttagtggagatataatatgaacatattagtaactggtggtgctggatatatcggatctcatacggctattgaattactgaatgcaggtcatgagattatcgttctggacaatttcagtaatgcttcatacaagtgtatcgaaaaaataaaagaaattactcgacgtgattttataacaattactggagatgctgggtgtaggaagacactctccgctattttcgagaaacacgccatagatatagttattcattttgctggctttaaatctgtttcagagtctaaaagtgaacccttaaagtattaccagaataatgttggagtgaccattactttattacaggtaatggaagagtacagaattaaaaaatttatctttagttcatctgcgacagtctatggtgaaccagagataattccaattccagaaacagctaaaattggaggaactacgaatccatatggcacatcgaagtattttgttgaaaaaattctagaggatgttagttccacgggaaaactggatataatttgcttgagatattttaatcctgtcggtgctcattctagtggtaaaataggtgaggctccatctggtatccctaataatcttgttccttatttattggatgttgcgagtggtaaacgtgataaattatttatttatggcaatgattaccctactaatgatggaacaggtgtaagggattttattcatgttgttgacttagcgaaaggtcatttggctgcaatgaattatttaagtatcaattcgggatataatatctttaatcttggtacaggaaaaggttattcggtacttgaattaatcactacatttgaaaaattaacaaacattaaggtcaataaatcttttatagagagaagggcaggggatgttgcgtcttgttgggctgatgcagataaagctaattctttattggactggcaagccgaacaaactctagaacagatgttattggactcgtggcgttggaaaaaaaattatccagacggattctgaatataaaaggtttcagttttatgaatcaatcagagcagagaaaaaaaatactggttcttacacctcgctttccctaccctgtcattggaggggatagattaagagtctatatgttatgtaaagaactttccaaaaaatatgatcttattcttctgagcttatgtgatcaaccactagaacttgaaataaatataaatgactcggtcttcaaagaaattcatcgtgtctatctaccaaaatataaatcatattataatgtattaaaagctttggttacgcaaaaaccgttgcaaattgcttattatcaatcggacacatttaagaataaatacaataaattaattaaacaatgcgatgcagtattttgtcatctgataagagttgctgattatgttaaggatacagacaagttcaaaattcttgatatgacagatgcaatatctttgaattacagtcgcgttaaaaaattagcaagtaaaaaaagtttgcgtgcaattatttattctctggaacaaaaaagattagaatcatatgaacgttctgtggcgaatctttttgatttgaccacttttatttcatccgtagaccgtgactatctctaccctaatctgggcagtaatatccatatagtcaataatggggttgatacatcagccttgagatatataaaaagagaaataaaaatcgataagcctgtggaacttatatttatcggaaatatgtattctttacaaaatatggatgctgcaaaacattttgctaagaatattttaccttgcttgtatgatgagtttaatattatttttaaagtgattggtaagatctcagaaactaataaaaatatattaaattcatttaaaaatacaattgctttaggtactgttgatgatatcaattcttccgcttctacagggcatataggtatatgtcctgttcgtcttggagcaggcgtacaaaataaaattcttgaatacatggctttaggtttaccatgtattacatctagcattggttatgaaggtattaatgcaaaatcaggtagcgaaatttttgttgcagatacagtagagcaatataaaaacgtactaagagaaataatttacgattataatcgttatactgaagtggctgaaaatgcccgtagttttgtagaaaataatttttcttgggaatcaaaagttgccaatttaatgaatacattagatgagaaattatatgaacaataataaaattattacacctatcattatggctggtggttcaggcagtcggttgtggccactatcaagaattctctatccgaaacaatttcttagcctaatcggtagtcataccatgcttcaaacaacggctaatcgtctggatggtttggattgtaccaacccttatgtcatttgtaatgaacaataccgctttatagttgctgaacagcttagaaaaatcgatagattgacttcaaagaatatcatccttgagcctgttgggcgtaacactgcccctgcaattgcattagcggcgttgctgatgtctaagtctgataaaagtgcagatgatcttatgctcgtactggctgcagatcacgttatacacgatgaagaaaaattttgtaacgctgttagatcggcaattccatacgctgctgatgggaaattggtaacatttggtataattccagacaaagcagaaactggttatggttatatacatcgaggacaatatattaatcaggaagattcggatgcatttatagtgtcatcatttgttgaaaagccaaatcatgagacagccactaaatatcttgcttccggtgagtattattggaatagcggtatgtttttgtttagtgcaaatcgttatatagaggaacttaaacaatttcggcctgatattttatccgcttgtgaaaaagcaattgcttcagcgaactttgaccttgattttgtgcgtttagatgaaagttctttctctaagtgccctgaagaatcaattgattacgctgtaatggaaaaaacaaaagacgcaattgttattccaatggatgctggctggagtgatgtcggttcatggtcttctctttgggaaattaatgataaagactcagacggcaacgtaatagttggggatattttctctcatgaaacaaagaattctttcatatatgccgaatcgggaattgttgctacagttggagtggaaaatttagttgttgtccaaacaaaggatgctgttcttgtctcagagagaaataaagttcaggatgtaaagaaaatagtagaacaaattaaaaattcaggtcgtagcgagcattatgttcatcgcgaagtatatcgtccttggggtaaatatgattccattgacacaggggagcgttatcaggtcaaacgtataacagtaaatcctggtgaaggactttctttacaaatgcaccatcatagggcagaacattggatcatagtttctggaactgcaagggtgactataggttctgaaactaagattcttagcgaaaatgaatctgtttacatacctcttggtgtaatacactgcttggaaaatccagggaaaattcctcttgatttaattgaagttcgttctggatcttatttagaagaagacgatgttatccgttttcaggaccgatatggtcgtagctaaatttttgataatgtaacgttagtagaagagcgctaatatttttagttaatctgtaataagtattatttgtttaaggtatatcatgtcgagtttaccctgctttaaagcctatgatattcgcgggaaattaggcgaagaactgaatgaagatattgcctggcgcattggtcgcgcttatggcgaatttctcaaaccgaaaaccattgtgttaggcggtgacgtccgactcaccagcgaaaccttaaaactggcgctggcgaaggggttacaggatgcgggcgtcgatgtgctggatattggcatgtccggcaccgaagagatctatttcgccacgttccatctcggcgtggatggcggcatcgaagttaccgccagccataacccgatggattacaacggcatgaaactggtgcgcgaaggggctcgcccgatcagcggtgataccggactgcgcgacatccagcgtctggcagaagccaacgactttcctcccgttgatgaaaccaaacgcggtcgctatcagcaaatcaatctgcgtgacgcttacgttgatcacctgttcggttatatcaacgtcaaaaacctcacgccgctcaagctggtgattaactccgggaacggcgcggcgggtccggtggtggacgccattgaagcccgctttaaagccctcggcgcacccgtggaattaatcaaagtgcacaacacgccggacggcaatttccccaacggtattcctaacccgctactgccggaatgtcgcgacgacacccgcaatgcggtcatcaaacacggcgcggatatgggcattgcctttgatggcgattttgaccgctgtttcctgtttgacgaaaaagggcagtttattgagggctactacattgtcggcctgctggcagaagcgttcctcgaaaaaaatcccggcgcgaagatcatccacgatccacgtctctcctggaacaccgttgatgtggtgactgccgcaggcggcaccccggtaatgtcgaaaaccggacacgcctttattaaagaacgtatgcgcaaggaagacgctatctacggtggcgaaatgagcgcccaccattacttccgtgatttcgcttactgcgacagcggcatgatcccgtggctgctggtcgccgaactggtgtgcctgaaaggaaaaacgctgggcgaactggtgcgcgaccggatggcagcgtttccggcaagcggtgagatcaacagcaaactggcacaccccgttgaggcgattaaccgcgtggaacagcactttagccgcgaggcgctggcggtggatcgcaccgatggcatcagcatgacctttgccgactggcgctttaacctgcgctcctctaacaccgaaccggtggtgcggttgaatgtggaatcgcgcggcgatgtaccgctgatggaagaaaagacaaaacttatccttgagttactgaacaagtaattcagtaatttcatataaatgggttttaaaaaacggaaaagatgagatatccggtgtggtatatccaaggtaatgctattcagtatctctatgagtgagttaacatctataccacatttaagccgcacacttcgggatccccatatgaatatcctccttagttcctattccgaagttcctattctttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtatcgataaagaaggtgtgttccataccgaatggctggattaa

SEQ ID NO: 14 - DNA, 15027 bp, synthetic construct
  misc_feature 1..15027: note = example O8 rfb locus nucleotide sequence - O8-EPA production strain stLMTB11734
  source 1..15027: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcgatcgcttaagatctaggatttcattatgttacttcctgtaattatggctggtggtaccggcagtcgtctctggccgatgtcacgcgagctttatccgaaacagttcctccgcctgttcgggcagaactccatgctgcaggaaaccatcacccgactctcgggccttgaaatccatgaaccgatggtcatctgtaacgaagagcaccgcttcctggtggctgaacagctacgccagctcaataagctgtcgaataatattattcttgagccggtcgggcgcaacaccgccccggccatcgccctggcagcccttcaggccacccgcgacggcgacgacccgctgatgctggttctcgccgctgaccatatcatcaataaccagtcggccttccacgacgccatccgggtcgccgagcagtatgctgatgaaggtcatctggtcaccttcggtatcgtgccgaatgccccggaaactggctacggttacattcagcgcggcgtggcgctcaccgatagtgcccattccgcgtaccaggtggcccgctttgtggagaagccggatcgcgagcgcgccgaggcttacctcgcctccggggagtactactggaacagcggcatgtttatgttccgcgccaagaaatacctcatcgagctggccaaataccgtccggatatcctggaagcctgccaggctgcggtgaatgccgccgataatggcagcgatttcatcaatatcccgcatgatattttctgcgagtgcccggatgagtccgtggactatgccgttatggagaaaaccgccgatgcggtggtggtcggtctcgatgctgactggagcgacgtcggctcctggtccgcactatgggaggtcagcccgaaagacgagcagggcaatgtcctcagcggtgacgcgtgggtacacaacagcgaaaactgctacatcaacagcgacgagaagctagtggcggccattggcgtagagaatctggtgattgtcagcactaaggacgccgtgctggtgatgaatcgcgagcgttcccaggacgtgaagaaggcggtcgagttcctcaagcagaaccagcgcagcgagtacaagcgccaccgtgagatttaccgcccctggggccgttgcgacgtagtggtccagaccccgcgcttcaacgtcaaccgcatcacggtgaaaccaggcggtgccttctcgatgcagatgcaccaccatcgcgccgagcattgggttattctcgccggcaccggtcaggtgactgtcaacggtaagcagttcctgttgtccgagaaccagtccacctttattccgattggcgccgagcactgcctggaaaaccctggctgtattccgctggaagtgctggagatccagtcgggggcgtaccttggcgaggacgacattattcgtattaaagaccagtatggtcgttgctaattattttcgggacaagacgcagaatgacacagttaacttgttttaaagcttatgacatccgtggtgaactgggtgaggaactgaacgaggacatcgcctaccgtatcggtcgcgcctacggcgaatttctgaaacccgggaagatagtggtggggggcgatgtgcgcctcacaagcgagtcgctgaagctggcgctggcccgcgggttaatggacgccggtaccgacgtgctggacatcggcctgagcggtaccgaagagatttactttgccaccttccaccttggggtagatggtggcatcgaggtgaccgcgagccacaatcctatgaactacaacggcatgaagctggtgcgcgagaatgcgaagcccatcagcggcgacaccggcctgcgggatatccagcgcctggcggaggaaaaccagttcccgccagtggacccggcgcgtcgcgggaccctgagcaagatatcggtactgaaggagtatgttgaccatctgatgagctacgtggacttctcgaacttcacccgtccactgaagttggtggtgaactccggaaacggggctgcggggcacgtgattgatgaggtggagaaacgcttcgcggcggctggggtgccggtaacctttatcaaggtgcatcaccagccggatggccatttccctaacggtatcccgaatccgctgctgccggagtgccgccaggataccgccgacgcggtgcgcgagcatcaggccgacatggggattgcctttgacggcgacttcgatcgctgcttcctgttcgatgacgaagcttcgtttatcgaggggtattacattgtcggcctgctggctgaggcgttcctgcagaagcagccgggagcgaaaatcattcacgacccgcgcttgacgtggaacacggtagacatcgtgacccgcaacggcggccagccggtgatgtcgaagacggggcatgcgttcatcaaggagcggatgcgtcaggaagacgctatctacggcggggagatgagtgcgcaccattacttccgcgatttcgcctactgcgatagcgggatgatcccgtggctgctggtggcggagctgctgtgtctgaagaacagctcgctgaaatcgctggtggcggaccgccagaaggcgttccctgcgtcgggagagatcaaccgcaagctaagtaatgctgctgaggcgatcgcccgcatccgggcgcagtatgagccggcggctgcacacatcgacacaacggacgggatcagtattgaataccctgaatggcgctttaacctgcgcacgtctaacaccgagccggtggtgcgtctgaacgttgagtccagagctgatgtggcgcttatgaatgaaaaaacgaccgagctgttacacctgttaagcggggaataaggtgagagatttactaacgacgatttatcgttatcggggatttatctggagcagtgttaaacgtgattttcaggcacgctatcaaactagtatgctgggcgcactatggctcgttttacaaccgctctctatgattctggtctataccctggttttttccgaggtgatgaaggcaagaatgcccgataataccgggtcgtttgcctatagtatttatctctgttccggggtactgacctggggattatttactgagatgctggataaaggtcagagcgtatttattaacaatgctaatctgatcaagaaactcagttttccgaaaatctgtctgccgatcatcgtgacgttatcggcggtgctaaatttcgcgattattttcagtctgtttctaatttttatcattgtcaccggtaacttccccggctggctctttctctcggtgataccggtcctgcttttgcagatcctgtttgccggtgggctggggatgatccttggtgtcatgaacgtctttttcagggatgtggggcaactggttggcgttgcgctgcaattctggttttggttcacacccattgtttatgtactgaattcattacctgcatgggcaaaaaatctgatgatgtataacccgatgactcggatcatgcaatcttatcagtccatcttcgcctatcatctggcccccaactggtattcgctatggccagtattggctctcgccattattttctgcgtcatcggtttcaggatgttccgcaagcatgcggcggatatggtggatgaattataatgagttatatcagagtaaataatgtcggtaaggcgtatcgccagtatcactcaaagaccgggagactgatcgaatggttatcccctctgaataccaaacgccataatttgaaatggatcctccgcgatattaatttcgaagtcgctccgggcgaggctgtcggtattatcggtatcaacggtgcaggcaagagtaccctgcttaaactcataaccgggacgtccaggccgacgactggagaaattgaaatctccggacgtgtcgctgcattactcgaattggggatggggtttcattctgatttcactggtcggcagaatgtttatatgtctgggcaactgttggggttatcgtcagagaaaataactgaactgatgccgcaaattgaagagtttgctgagattggggactatatcgatcaacctgtgcgcgtctactccagtgggatgcaagttcgattagcttttagtgtagcgacggctatccgtcctgatgtgctaattatcgatgaggcattatctgttggggatgcatatttccagcataaaagctttgagcgtattcgaaaatttcgtcaggaagggaccacgctgttgctggtatcccatgataaacaagcgatccaaagcatttgcgaccgggccattttattgaataaaggccaaattgaaatggaaggtgaacctgaagcagtgatggattattacaatgctcttctggccgataaacaaaatcagtccattaaacaagttgagcataatggtaaaacgcaaactgtttcaggcactggtgaggtgactatctctgaggttcatcttctcgatgaacagggcaatgtgactgaatttgtttcggtagggcatcgtgtcagcttgcaggtcaacgttgaggtcaaggacgatattcctgagcttgttgtcggatatatgattaaggatcgacttgggcagccgattttcgggaccaatacgtaccatctcaatcagacactcacctccctgaaaaaaggagaaaagcgttcgttcttattttctttcgatgcgagattgggggttggctcctattctgtcgctgtcgcgttgcatacttccagtacgcacctcggcaaaaactatgaatggcgcgatctggccgtggtattcaacgtcgttaacacggaacaacaagagtttgtcggcgtgtcctggttgccgcctgaactggagatttcttaatgggttcgtcgttttatcgttcatttgaagaacgacacagaggttcggttgaagaaatcaagcgccgcttgagtttttatttaccttttcttgcaggtctgaaggacatttatcctgatggcgtgattgcggatattggttgcggacgtggcgaatggttggagatcctgactgaaaatggcattgcgaacatcggcgtcgatctcgatgatggcatgctggcgcgcgccagggaggccggactgaatgtgcagaaaatggattgtctgcagtttttgcaaagtcaggcggatcagagcctgatagcgttgaccggttttcatattgctgagcatttgccgtttgaggtcctgcagcaactcgccatgcataccctacgggtgctgaaaccaggtggtttgctgatcctcgaaacgccgaacccggagaatgtaagcgtcggcacctgttcattttatatggatccaacgcataatcatcctctgccaccgccactgcttgagtttttacctattcattatggttttacccgagcaattaccgttcgtctgcaggaaaaagaggttcttcaatctccggatgcagccgttaatttggtcgatgtactcaaaggggtgagccccgactacagcatcattgctcagaaagcagcgccaacagatattcttgaacgctttgacaccctgtttacccagcagtacggtctgacgctggatgctctgagcaaccgttacgatgcgattttgcgccaacagttttcgtccgttgtctcacggctggagacgttgaaccaaacctatatgcaacagataagccaaatgtcagagactattcagacgttgcaaggtgaggttgacgatctgagtcatgtcatcgatcagaaccatcagcttcatcagcaaatggcggatttacataacagtcgttcatggcgtattactcaaccactacgctggttgtctttgcaacgtcaattattacgtcaggaaggggctaaagtgcgagcccgtagggctgggaaaaaaatattgcgcaaagggatggcgctctcgctggtctttttccatcgttaccctaagtctaaggtttatctgtttaaggttctgagaaaaactggctgctatacattgctacaacgtttgttccaacgcgtaatgctggtgcaatctgacacgatgatgatgcagtccagaagatatgatgtgggtactgaagaaatgacaagtcgcgcgatgagtatttataacgaattaaaaaataaaaatacggagaaataacgatgcgtattgtcatagatttacaaggcgcacagacggaaagccgctttcgtggcatcggtcgttatagtatcgcaatcgccagaggcataatcagaaataacagccggcatgagattttcatcgcgctatccgccatgctggatgagtcgattgcaaatattaaggcgcaatttgccgatctcctgccggcagaaaatatagtcgtatggcatgccgtaggccctgttcgtgcgatggaccaaggtaatgaatggcgtcgggagagcgcagaactgattcgggaagcgtttcttgaatcattgtgtccagatgtcgttttcattacgagtttgtttgaaggtcatgtcgacgatgcggctacatcggtacacaaatttagtcgtcagtataaagtagccgtactgcaccacgatcttatccccctcgtgcaggcggaaacctatctgcaggacgatgtatacaaaccctactatttacagaaagttgagtggttaaaaaacgctgaccttttgttgactaactctgcttataccgcacaggaagcgatcgagcatctgcatttacagggcgatcatgtgcagaatattgcagccgcagtcgattctcagttttgtatggcggaggtggcagcgagcgaaaaagagaccgtccttggccattacggtattcagcgcgagttcatgttgtatgcgcccggaggatttgactcaaggaaaaactttaaacggttgattgaggcctatgccgggctcagtgatgccttacgtcgcagtcatcaactggtcatcgtcagtaagctttccatcggtgatcgtcagtatctggaatcccttgcgtcaggtaatggtttacagcagggcgaactggtactcactggttatgtgccggaagatgagctgatccagctctatcgcctatgtaagctgttcatctttgcttcactacatgaaggttttgggttgccggttctggaagcaatgtcgtgcggtgcgccggtgattggctcaaatgtcaccagtattcctgaagtcatcggtaatcctgaggcattattcgacccgtattctgtctcttccatgagggataagatcgcgcaatgtttgactgatgataccttcctcgcgcgtctgaaagaaatggcgcagcagcaagcgcgtaatttctcttgggataaagctgcggtgactgctctggaagctttcgaaaagatcgcggtagaagacaccggtactgcgcaggttttgcctgaagctttgattcagaagatccttgctatctcacaagggcagccagatgaccgcgatctgcgcttgtgcgcaacggccattgattacaatctgaaaacggcagaactttatcaaatcgacgataaatcgctgaactggcgtgtggaaggcccattcgatagctcatatagtctggcgttggtcaaccgcgaatttgcccgggcactctcagccgatggtgtagaggttttattgcattccactgaaggaccaggtgattttgccccagatgcctcgtttatggcacagtcggaaaatagtgatcttctggcattttataatcaatgtcagacccgcaagagtaacgaaaagatagatattattagcagaaatatctatccaccgcgggttaccaaaatggatgccaaagtaaaattccttcattgttatgcttgggaagaaacgggctttccgcaaccgtggatcaatgaatttaatcgggaacttgacggagtgctgtgtacttcggaacatgttcgtaaaatactgattgataacggactgaatgtgcccgcatttgttgttggcaatggctgtgaccattggctcaatatcccagccgagacgacaaaagatgtggatcacggaacattccgtttcctgcacgtctcttcttgtttcccacgcaaagggatacaggcaatgcttcaggcttgggggaaggcgttcactcgtcgtgacaatgttatcttaatcattaagacttttaacaatccgcacaatgaaattgacgcatggctggctcaggcccaggctcaattcatagactatcccaaagttgaagtgatcaaagaggatatgtcagccaccgagcttaaagggctttatgaaagctgtgatgttttggttgctccaggttgcgctgaaggctttggtttacctattgctgaagcaatgctgagtgggctaccggctatcgtcaccaattggagcgggcaacttgattttgttaattcacaaaattcatggctggttgactatcagttcactcgggtaaaaacgcactttggtctgttttcctcagcctgggccagtgtggatattgacaacttaacagatgcattaaaagcggcagcctcaaccgataaatcagtgctgcgtgacatggccaatgctggtcgcgagcttcttctgcagcagtttacctggaaagcggtggctgatcgttcttgccaggcggtcaagactctgcgtgcgcatattgatattgcacagcatcgggcgcgcattggctgggtgacgacctggaacacgaaatgtgggatcgcaacctattcccagcatctggtggaaagcgcacctcatggcgcggatgttgtttttgctccccaggtcagcgctggcgatcttgtgtgtgcagacgaagagtttgtacttcgcaactggattgtaggtaaagagagcaactatctggaaaacctccagccacacattgatgctctgagactcgatgtcattgtgatccaattcaactatggattctttaatcatcgagaactgtcggcgtttattcgtcgccagcatgacgccggtcgttcagttgttatgacgatgcactcaactgtggatccgctggaaaaagagccgagctggaatttccgtcttgctgaaatgaaagaggcgctggcactttgcgaccggttgttggtgcattcgattgccgatatgaaccgccttaaagatttaggcttaactgcgaatgttgctttattcccgcacggtgttatcaactactccgcagcgagcgtcacacgtcaacagcagtctttaccgctaattgcgagctatggcttctgcttaccgcataagggcctgatggaactagtagaatccgtccatagactcaagcaagccggtaaaccggttcgtttacgactggtgaacgcagagtatcctgttggggagtcacgcgatctggtggcagagcttaaagctgctgctcagcggttaggtgttaccgatctgattgagatgcataatgatttcctacctgatgcggagagtctgcggttgctttcagaagccgatcttctgatttttgcttatcagaatactggggagtctgctagcggggcggtacgttatggtatggcgactcaaaaacctgttgcggtaacgcccctggcgatatttgatgatttggacgatgccgtctttaaatttgatggatgcagcgtcgatgatatcagtcaggggattgaccggatcctgaattccatccgtgaacagaactcttgggcaaccaggactcaacaacgtgccgatgcatggcgggaacaacatgattatcaagctgtttcacgccgtctggttaatatgtgtcaaggcttagctaaagctaaatattttaaataaaaatatctctcttgtattttttgcctttgaatacaagaggggttagataatgtgtcatttattatgaaaattatttttgctactgagccaattaaatacccattaacgggcatcggtcggtattccctggagctggttaagcggctggcggtcgcccgcgaaattgaagaattaaagctatttcacggtgcgtcgtttatagaacagatccctttggtggagaataaaagcgataccaaagccagcaatcatggtcgtctgtcggcgtttctacgccgacagacgctgttgattgaggcttatcgcttgctgcatccgcggcgccaggcgtgggcattgcgcgactataaggattatatctaccatggccccaatttttatctgccgcataaactggaacgcgccgtgaccacgtttcatgacatatccatttttacctgcccggaatatcatccaaaagatcgggttcgctatatggagaagtccctgcatgagagtctggattcggcaaagctgatcctgaccgtttctgatttctcgcgcagtgaaattatccgcttgttcaactatccggcggagcggatcgtaaccaccaagctagcctgcagcagtgactatatcccacgcagcccggcagagtgtctgccggtactgcagaaatatcagctggcgtggcaggcctacgcgctatatatcggcactatggagccacgtaaaaatatccgaggcctgctgcatgcctatcagctgctaccgatggagatccgcatgcgctatccgctaatccttagcggctatcgcggctgggaagacgatgtgctgtggcagttagtcgagcgcggtactcgggaaggctggatccgttacctcggatatgttccggatgaagacctgccgtatctgtacgcagcggccagagtctttgtttatccctccttctacgagggattcggtttacctattcttgaagcgatgtcttgcggtgtgccggtagtatgctccaatgtcacctctttgcctgaggttgttggcgatgccggcctcgttgccgatcctaatgatatagacgcgattagcgcgcaaattttgcagagcctgcaagatgatagctggcgggaaatcgccaccgcgcgcggtcttgctcaggcgaaacagttttcgtgggagaactgtgcgacacagaccattaacgcctataaattactctaagggtgtcagttgagagttctacacgtctataagacttactatcccgatacctacggcggtattgagcaggtcatttatcagctaagtcagggctgcgcccgccggggaatcgcagccgatgttttcacttttagcccggacaaagatacaggtcctgtcgcttacgaagatcatcgggtcatttataataaacagctttttgaaattgcctccacgccgttttcgctgaaagcgttaaagcgttttaagctgattaaagatgactacgatatcatcaactaccattttccgtttccctttatggatatgctgcatctttcggcgcggcctgacgccaggactgtggtgacctatcactctgatatagtgaaacaaaaacggttaatgaagctgtaccagccgctgcaggagcgatttctcagcggcgtagattgcatcgttgcctcgtcgcccaattacgtggcttccagccagaccctgaaaaaatatctggataaaacggtggtgatcccgtttggtctggagcagcaggacgtgcagcacgatccgcagagggtcgcgcactggcgggaaactgtcggcgataagttctttctcttcgtcggcactttccgctactacaaagggctgcatattctgatggatgccgctgagcgtagccgactgccagtggtggttgtagggggcgggccgctggaatcggaagtgcggcgtgaagcgcagcagcgcgggctgagcaatgtgatgtttaccggcatgctcaacgacgaagataagtacattctcttccagctctgccggggcgtggtattcccctcgcatctgcgctctgaggcgtttggcattacgttattggaaggcgcacgctttgcaaggccgctgatctcttgcgagatcggtacaggtacctctttcattaaccaggacaaagtgagtggttgcgtgattccgccgaatgatagccaggcgctggtggaggcgatgaatgagctctggaataacgaggaaacctccaaccgctatggcgaaaactcgcgtcgtcgttttgaagagatgtttactgccgaccatatgattgacgcctatgtcaatctctacactacattgctggaaagcaaatcctgagcggccgcgagctcgtcgactcgaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 15 - DNA, 11283 bp, synthetic construct
  misc_feature 1..11283: note = example O15 rfb locus nucleotide sequence - O15-EPA production strain stLMTB11738
  source 1..11283: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcatgagcaaaactaaactaaatgttctttaccttgcaataagtcagggtgccaattacctactgccattattaatttttccttatcttgttagagtcattggtgtatcgaattttggtgatctgagtttttcattgataactatacaagtgttgttaatggttgttgaatatggttttggatatagtgggacaagagaaatagcactaaataacgataaaaaataccattctgaatttttttgcggtgtggtgcttgctcgttttatattaatgctaattgcagctataatactcataatactctgttttttttatgtttttaacgacgttaagtctttgttatgtgttggttttctgtccgtaattgcaggtgttttcaatccaaattggtttttgcaaggtaaggaaatgatgagtgtgatggctgtgctgtcactattttcacgaggcatagcagtcgttgcagtttatctaattataaaacccgcaacgccgatgtacatcagtgccttattattgagcatgccatatattttgtattcattctgtggcgttgcctacttacttattatcaaggagatttttttatgtaggccaccgataaagaaaattcaagtaattttaaaaaatggatttcattttttttgttcaacacttgcgactagtgcatacacaatgttgacccctcttgtattgggtggcgtatctggaaagtttgatgtaggcatctttaactcagctaacatgatcaaacaaggtttggctggacttgcatcaccattagtccaagctttttatccaagaattaacattttgcaaagagagaatccatatattgcaaacttaaaatctagaatgattcttaaatacttgcttgttttttacatggctttagcaataccatttttactttttgccaaccaattatcattattaatattcggcatgaaaggtgaagtaattgcaggtgcaatgcaattaatgacattgcttcctatattcataggttttaatacagttgtcgggttacttgtattagtacctaatgggatgcaaaaacagtatttcaaatctattttcctaggaactattacttgtttaagcatagtttatccagcatgtaaatattatggagcaacgggtgcgattgtgagtcttattgtagctgaaattttcgttggcatgggaatgcttaaacaattcattaaagtaaataaaaccgtatgtaggcctcataaattatgaatatctcggtaataatatctgtttggaaacgcccagttcaattagaattgattctctctgagctcgattctcaggctaaagacaatagtctacacctagaagtaattgtttccgatagtcatagtggtaaagaaattgatgatgtagttgctgataatattcataaaaagaaaaatattaatattatccatcaacatactaaaaatatactctccgctaagcgcaatttcggagcatccctagcccatggggattatttaatatttcttgatgatgattgtatacccgcaagtggatatatatcatcgttgctgaactatttaaaaaaaatgaatagtaaaagcgttttatgtggggaagttagattcgaaaatgaactcattgagaccagcaattactatcgctacaggaactctttacaccctaagtttagtgatagtcctgatatctctatgaatgcctggacttttgtcgcaatgaattgtgttcttgatagaaaggcattttcatcaggtatagtttcatataatgaaaattttattggttatggttgtgaagatcatgagtttgggtggcaacttgaaaaaaatgacttcaaaattatttttgctgattttaaaatattacatcacgaatacagtggcgatatagaaggatatacaaaaaaaattcgtgctacagcacgtgatggtatgaatgtattaagcaaagtaaggcctgaaatgttttctactaataaaaaattattcctagttgagaaaatatttagtaaacacaaaacgtttagtaaaatatgccaatcaatatttttcaataaatttatttttaaaaaaataatacaatttttaaaaaaaacagatgcaaataaaaaactctatttcccaattctttacagatatgtgttgatttcggcatatatacatggtattggagagcgtggcacctcaaaaacagatgatttgcttaagaactggtatatatagatgatgctatcttcatttattaagacatttgtatggaaggtaaaaaacaatgaagtataatgcattgatggcttttttattattttttgttgttttttttagattgtcgctgataatacctttcttatatttggcatttattcctgcattttttggtattatgtatttagtgcgtaattttatgattactatgggcaatggattggtatctatagatcgtaaaaatttgttgctgttatctatattcataattatttttttattttgtttggttttcgatttgtttcaaaaaagccattcttttcaaagttattttaccgttagattatttatgttgtttttattttcatttgttcctgcgtattatttagtaaatagattcataaagggtgacttgaaattaatggagcgaatattagtgtattctctctgggttcaaatagttattttttttggtatgtatataagtccagagttaaaaagattgttatatactttctttggtatgtctgactctgttaatctttgggaacaaaatgctaaagtaagaggatttgggttgtcgggtgaaataaatttcatgacaccatttttgatgatctatatgtcattttttatgatgaaaaggcgttatgctttaattactttaatttgtctgactcaaatcgtaaattctaacatggctgtgattgcagccattattggtatcggttgctctagacttaatattaatataaaaattgcaacagtattgattttgggagttttagtttatagcttaggagcggtgttctttcctcgattttatgatgagttcgtttctggagatggcacaagaactctggatatcttattacagcaacatgtgtttgttgtaggtaatttagatttttttaatattatatttggattacagcaaaacatatcttcatcaatccccgatattaaacaaagttcggatatgggctgggttatactgtttaattacggtgggttaacatttattacactctttttatttttaatctttactatttctattgcgacatttggaatgacatatcaagcaattatatggatgttaattgggataattttcaataccaaaggtttagttttaggatctaacggctatttctttctatcttttatatatatgtttttgaatagagtaacacttagtggacagagttcaattactaataagttaggtcaagtaagtaaatagcttccagagtatatttgtcaatgatttgaggttcggttattatgttttcatctaaaacactgttaattactggtggtactggctctttcgggaatgctgtattaaatagatttcttgatacagatattgcagaaatccgtatatttagtcgtgatgaaaaaaaacaagatgatatgcggaaaaaatacaataatcaaaaattaaagttctatattggtgatgtcagagattaccgtagtattttgaatgcgactcgcggtgttgattttatatatcatgcagcggcacttaagcaagttccatcatgtgaatttcatcctatggaagccgttaaaactaatatccttggtacggaaaatgttcttgaagcagctatagcgaatgaagtgaagagggttgtatgcctaagtactgataaagctgtatacccgattaacgcaatgggtatttcaaaagctatgatggaaaaggtcatggtcgcgaaatcccgtaatgttgatcgcaataaaacagtaatatgtggtacccgttatgggaatgttatggcatctcgcggttcagttattccattatttgttgatcttattagagcgggcaagccactcacaataactgatcctaatatgacccgctttatgatgactcttgaggatgcggtagatttagttctttatgcgtttgaacatggtaataatggtgatatctttgtgcaaaaagcacctgcagcaactattgacacattagctattgctttaaaggaattactaaatgttcctgaccatccggtaaatgtcattggaacgcgtcatggcgagaaattatatgaagctctacttagtcgtgaggaaatgatcgctgctatagatatgggcgattattaccgtgtcccgccagatcttcgtgaccttaattatggcaaatatgttgagcaaggtgatagccgaatatctgaaatagaagattataactctcataatactcaacggttagatgttgaaggcatgaaagagctcttgctaaaattagcctttattcgagcaattcgtgctggtgaaaaatataatctggattcatgatatgaaaatattagttactggtgcaaatggttttattggtcgtaatttatgtttgaggcttgaggaacttggttataaagatcttattagaattgatcgagaatcaacgaagcaagatcttgaacaaggcttacaggatgccgattttatttatcacttagctggtatcaatagacctaagactgatgatgagtttatttctggaaacagtgatttaacaaagcatatagttgagtatctcctttctattggtaagaatacaccaattatgctaagttcttcgatacaagctgaacttaataatgcttatggggttagcaaagctgtagctgaaagctatgtcgaaaaatatgctgctgctagtggttcttcgtattatattttcagatatccaaacgtttttggtaaatggtgtaagccaaactataattcttttatagcaactttttgctacaatatttccaatgatattgagattactatcaatgatgcagcagcgccagtcaatctggtctatattgatgatgtttgtactgatgctatagctcttctctctgggacggttgaaagtggatataaagttgttgcaccaatttattcaacaacagttggtgaagttgcagaattaatttatagcttcaaaaatagccgttccaccctgatcacagaggctgtcggggcgggatttacccgtgcattgtattctacatggctgagttatttaccagcagagaagtttgcgtacaaggtacctttttatggggatgcccgcggagtcttttgtgagatgttgaaaacgccttcagcggggcagttttcattttttactgctcaccctggtattacgcgtggcggacattaccatcacagtaaaaatgagaagtttttggtcattcgaggtcaggcatgctttaaatttgaacatgtgattaccggtgagcgatatgaactgaaagtttcatcgggtgagtttaagattgttgaaacagttcctggttggacacatgacattacaaatattggaactgatgaattaatagtcatgctctgggcaaatgaaattttcaaccgtgatgagcccgatactattgcgagacctctataatgaaaaaattaaaagttatgtctgttgttggaacccgtcctgagattatccgtttgtcgagggttcttgctaagtttgatgaatactgcgagcatattattgtccatactggtcaaaattatgattacgaattaaatgaagtgttcttcaatgacttgggtgttcgaaaacctgattattttttaaatgcagcgggtaaaaatgcggcggaaaccattggtcaggttattattaaggtagatgaagtattagaaatcgaaaaacctgaagcaatactggtattgggcgatacgaattcatgtatttctgccattccggccaaacgccgtaaagtgcctatatttcatatggaagcaggtaaccgttgtttcgatcaacgcgtgcctgaagaaaccaacagacgtattgttgaccatacggctgatatcaatatgacctacagtgatattgctcgtgaatatctcttggctgaaggtatcccagctgatcggatcataaaaactggtagccctatgtttgaggttctttcatattatatgccccaaattgatggttcagatgtgctatcgcgtttgaatctacagtctggtgagttttttgtagtaagtgcgcatcgtgaagagaatgttgattctccaaaacagctcgtaaagcttgcgaacattctaaatactgttgctgaaaaatataatcttccagttattgtctccacacacccaaggacacgtaaccgaatccgtgagcaaggaattgaatttcattcaaatataaatctactgaaaccattgggtttccatgattataaccacttgcagaagaactcacgagctgtgctttcagatagcggtactatcactgaagagtcatccatcatgaatttcccagcggtaaacatccgggaagcgcatgagcgtccggaaggctttgaggaagcatccgtcatgatggtggggttagagtgtgaacgcgtattacaagcgctggatattctggcaacacaaccgcgaggtgaagtccgtcttttacgtcaggttagtgattacagcatgccaaatgtgtcggataaagttgtcagaattgttcactcttacacagattatgttaagagagtcgtctggaaagaatattgatgaaacttgctttaatcatagatgattacctgcccaacagtactcgtgttggtgcaaaaatgtttcatgaacttgctcaagaatttatccagcgtgggcacgatgttacggtaattactcctggtacgggcatgcaagaagagatttcttttgatacctttcagggggtaaaaacatggcgttttaaaagcgggccgctcaaggatgtaagtaaaattcagcgagcggtcaatgaaacgcttttgtcctatcgggcgtggaaagccatcaaaaaatgggtaaaaaaagagacctttgagggggtgatttattattcaccttccatattctgggggcctttagttaaaaaaattaaagctcgttgccaatgtcctgcttatcttattttaagagatatgtttccacaatgggtaattgatgcaggaatgcttaatgctggttccccaatagaacgctactttcgtctttttgaaaaaatatcttatcgtcaggcaaatcgtattggacttatgtctgataagaatcttgatgtttttcggaaagataataaaggctatccgtgcgaagttttgcgtaattgggcatccctaacaccaacgatcatacccaaggattatataccactacgtaagcgacttggcctagaggataaaaccattttcttctatggtggaaacataggtcatgcacaggacatgacaaacttgatgcgacttgtgagaaacatggcagcatatcctcaagctcatttcctatttattggccagggggatgaagttgaattaattaattcattagcatctgagtgggcattgacgaatttcacctatttgccctcggttaaccaagatgaatttaagttcattttgtcggaaatggatatcggcttgttttctctttccgctagacactcttcccataattttcctggtaagttattaggctatatggttcagtcgctacctattttaggtagcgtaaatgccggaaatgatttgctcgacattgtcaatcaaaataatgcgggattaatccatgtcaatggtgaggacgataaattatgtcaatctgcgctattaatgttgcatgatattgatgtgcgccggcaacttggttcgggggcgaatatattgttgaaagaacaattctccgttgagtctgcggcacagacgatagaaatgaggttggaggcatgcaatgcgattaattgataatgaccaactcgacgaattatatgatcaagccgggcaatcggaacgtttacgttcccaccttatgatgcacggctcgcatcaagaaaaggtacagcgtttacttattgcattagtaaagggcagctatgttgaaccgcattatcacgaacttcctcatcagtgggaaatgttcattgttatggaggggcaacttcaggtttgtttgtatggtagaaatggtgaggttataaagcaatttatagcaggagataatactggaatgagcattgtggagttttctccgggcgatatacacagtgtcgaatgcctatctccgcgtgctcttatggtggaagttaaggaggggccatttgacccttcttttgcaaaatcgttcgtgtgagcggccgcgagctcgtcgactcgaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 16 - DNA, 13435 bp, synthetic construct
  misc_feature 1..13435: note = example O16 rfb locus nucleotide sequence - O16-EPA production strain stLMTB11739
  source 1..13435: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagtgaaaatacttgttactggtggcgcaggatttattggttcagctgtagttcgtcacattataaataatacgcaggatagtgttgttaatgtcgataaattaacgtacgccggaaaccgggaatcacttgctgatgtttctgattctgaacgctatgtttttgaacatgcggatatttgcgatgcacctgcaatggcacggatttttgctcagcatcagccggatgcagtgatgcacctggctgctgaaagccatgttgaccgttcaattacaggccctgcggcatttattgaaaccaatattgttggtacttatgtccttttggaagccgctcgcaattactggtctgctcttgatagcgacaagaaaaatagcttccgttttcatcatatttctactgacgaagtctatggtgatttgcctcatccagatgaagtaaataatacagaagaattacccttatttactgagacgacagcttacgcgccaagcagcccttattccgcatccaaagcatccagcgatcatttagtccgcgcgtggaaacgtacatatggtttaccgacaattgtgactaattgctcgaacaactatggtccttatcatttcccggaaaagcttattccactggttattcttaatgcactggaaggtaaggcattacctatttatggcaaaggagatcagatccgcgactggttgtatgttgaagatcatgcgcgtgcgttatataccgtcgtaaccgaaggtaaagcgggtgaaacttataacattggtgggcacaacgaaaagaaaaacatcgatgtagtgctcactatttgtgatttgctggatgagattgtaccgaaagagaaatcttatcgtgagcaaatcacttatgttgctgatcgtccgggacacgatcgccgctatgctattgatgctgagaagattggtcgcgcattgggatggaaaccacaggaaacgtttgagagcgggattcgtaaaacggtggaatggtacctgtccaatacaaaatgggttgataatgtgaaaagtggtgcctatcaatcgtggattgaacagaactatgagggccgccagtaatgaatatcctcctttttggcaaaacagggcaggtaggttgggaactacagcgtgctctggcacctttgggtaatttgattgcttttgatgttcactctactgattattgcggtgattttagtaatcctgaaggtgtagctgaaaccgtaagaagcattcggccggatattattgtcaatgcagccgctcacaccgcagtagacaaagcagaatcagaaccggagtttgcacaattaattaacgcaacaagtgtcgaagcgattgcgaaagcagcaaatgaagttggagcctgggttatccattactcgactgattacgtcttccctggaaatggcgatatgccatggctggagacggatgcaaccgcaccactaaatgtttacggtgaaaccaagttagccggagaaaaagcgttacaggaatattgcgcgaagcatcttattttccggaccagctgggtctatgcaggaaaaggaaataacttcgccaaaacgatgttacgtctggcaaaagagcgtgaagaattagcggttattaacgatcagtttggtgcgccaacaggtgctgaactgctggctgattgtacagcacatgccattcgtgtcgcactgaataaaccggatgtcgcaggcttgtaccatttggtagccagtggtaccacaacctggtacgattatgctgcgctggtttttgaagaggcgcgcaaagcaggcattccccttgcactcaacaagctcaacgcagtaccaacaacagcctatcctacaccagctcgtcgtccacataactctcgccttaatacagaaaaatttcagcagaactttgcgcttgtcttgcctgactggcaggttggcgtgaaacgaatgctcaatgaattatttacgactacagcaatttaatagtttttgcatcttgttcgtgatggtggagcaagatgaattaaaaggaatgatgaaatgaaaatgcgtaaaggtattattttagcgggtggttctggtacacgtctttatcctgtgactatggctgtcagtaaacagctattacctatttatgataaaccgatgatctattacccgctctctacactgatgttggcgggtattcgcgatattttgattatcagtacacctcaggatactcctcgttttcaacaattgctgggtgacggtagccagtggggcctgaatcttcagtacaaagtgcaacctagcccagatggcctcgcgcaggcatttatcatcggtgaagagtttattggtggtgatgattgtgctttggttcttggtgataatatcttttacggtcacgatctgccgaagctaatggaggccgctgttaacaaagaaagtggtgcaacggtatttgcctatcacgttaatgatccagaacgctatggtgtcgttgagtttgataaaaacggtacggcaatcagtctggaagaaaaaccgttagaaccaaagagtaattacgccgttacaggtctgtacttttatgataacgacgtggttcagatggcgaaaaacttgaagccgtctgcacgtggtgagttagaaattacagatattaaccgtatttatcttgagcagggacgtctgtctgtcgcgatgatggggcgtggctacgcgtggctggacacggggactcatcagagtctgatagaagcaagtaattttattgcgacaattgaagagcgccagggattgaaggtttcctgtcctgaagagattgcatttcgtaaaggttttattgatgttgagcaagtaagaaaattagctgtaccactaataaagaataattatgggcagtatctttataaaatgacgaaggattcaaattaatgaatgtgattagaactgaaattgaagatgtgctaattctggagccaagagtatttggtgatgatagaggtttcttttatgagagctttaatcaatcagcatttgaacatattctaggctatccggtcagctttgttcaagacaatcactcacgttcatcaaaaaatgtactcagaggccttcactttcaacgcggcgagtacgcacaagataaacttgtacgctgcactcatggagcagtttttgatgttgctgttgatattcgacccaattcggtatcctttggtaaatgggttggtgttctgctttcagctgataataagcagcagttgtggataccaaaagggtttgctcatggctttttggttctgtctgatatcgctgaatttcaatataaaactacaaactattatcatcctgaaagcgattgtggaatatgttggaatgatgaacgcattgcaattgattggccccaaacatcagggttaatcctttcgccaaaagatgaaaggctctttacgttagatgagcttatcagattaaaattaattgcatgaatacgaataaattatctttaagaagaaacgttatatatctggctgtcgttcaaggtagcaattatcttttaccattgcttacatttccatatcttgtaagaacacttggtcctgaaaatttcggtatattcggtttttgccaagcgactatgctatatatgataatgtttgttgaatatggtttcaatctcacagcaactcagagtattgccaaagcagcagatagtaaagataaagtaacgtctattttttgggcggtgatattttcaaaaatagttcttatcgtcattacattgattttcttaacgtcgatgaccttgcttgttcctgaatataacaagcatgccgtaattatatggtcgtttgttcctgcattagtcgggaatttaatctaccctatctggctgtttcagggaaaagaaaaaatgaaatggctgactttaagtagtattttatcccgcttggctattatccctctaacatttatttttgtgaacacaaagtcagatatagcaattgccggttttattcagtcaagtgcaaatctggttgctggaattattgcactagctatcgttgttcatgaaggttggattggtaaagttacgctatcattacataatgtgcgtcgatctttagcagacggttttcatgtttttatttccacatctgctattagtttatattctacgggaatagttattatcctgggatttatatctggaccaacgtccgtagggaattttaatgcggccaatactataagaaacgcgcttcaagggctattaaatcctatcacccaagcaatatacccaagaatatcaagtacgcttgttcttaatcgtgtgaagggtgtgattttaattaaaaaatcattgacctgcttgagtttgattggtggtgctttttcattaattctgctcttgggtgcatctatactagtaaaaataagtatagggccgggatatgataatgcagtgattgtgctaatgattatatcgcctctgccttttcttatttcattaagtaatgtctatggcattcaagttatgctgacccataattataagaaagaattcagtaagattttaatcgctgcgggtttgttgagtttgttgttgatttttccgctaacaactctttttaaagagattggtgcagcaataacattgcttgcaacagagtgcttagttacgtcactcatgctgatgttcgtaagaaataataaattactggtttgctgaggattttatgtacgattatatcattgttggttctggtttgtttggtgccgtttgtgcgaatgagttaaaaaagctaaacaaaaaagttttagtgattgagaaaagaaatcatatcggtggaaatgcgtacacagaggactgtgagggtatccagattcataaatatggtgcacatatttttcataccaatgataaatatatatgggattacgttaatgatttagtagaatttaatcgttttactaattctccactggcgatttataaagacaaattattcaaccttccttttaatatgaatactttccaccaaatgtggggagttaaagatcctcaagaagctcaaaatatcattaatgctcagaaaaaaaagtatggtgacaaggtacctgaaaatttggaggagcaggcgatttcattagttggggaggacttataccaagcattgataaagggttatacggagaagcagtggggaagaagtgcaaaagaattgcctgcatttattattaagcgaatcccagtgagatttacgtttgataacaattatttttccgatcgctatcaaggtattccggtgggaggctacactaagcttattgaaaaaatgcttgaaggtgtggacgtaaaattaggcattgattttttgaaagacaaagattctctagcgagtaaagcccatagaatcatctacactggacccattgatcagtacttcgactataggtttggagcgttagaatatcgctctttaaaatttgagacggaacgccatgaatttccaaacttccaagggaatgcagtaataaatttcactgatgctaatgtaccatataccagaataattgagcataaacattttgactatgttgagacaaagcatacggttgttacaaaagaatatccattagagtggaaagttggcgacgaaccctactatccagttaatgataataaaaacatggagctttttaagaaatatagagagttagctagcagagaagacaaggttatatttggcgggcgtttggccgagtataaatattatgatatgcatcaagtgatatctgccgctctttatcaagtgaaaaatataatgagtacggattaatgatctatcttgtaattagtgtctttctcattacagcatttatctgtttatatcttaagaaggatatattttatccagccgtatgcgttaatatcatcttcgcactggtcttattgggatatgaaataacgtcagatatatatgcttttcagttaaatgacgctacgttgatttttctactttgcaatgttttgacatttaccctgtcatgtttattgacggaaagtgtattagatctaaatatcagaaaagtcaataatgctatttatagcataccatcgaagaaagtgcataatgtaggcttgttagttatttctttttcgatgatatatatatgcatgaggttaagtaactaccagttcgggactagcttacttagctatatgaatttgataagagatgctgatgttgaagacacatcaagaaatttctcagcatacatgcagccaatcattctaactacttttgctttatttatttggtctaaaaaatttactaatacaaaggtaagtaaaacatttactttacttgtttttattgtattcatctttgcaattatactgaatactggtaagcaaattgtctttatggttatcatctcttatgcattcatcgtaggtgttaatagagtaaaacattatgtttatcttattacagctgtaggtgttctattctccttgtatatgctctttttacgtggactgcctggggggatggcatattatctatccatgtatttggtcagccctataatcgcgtttcaggagttttattttcagcaagtatctaactctgccagttctcatgtcttttggttttttgaaaggctgatggggctattaacaggtggagtctctatgtcgttgcataaagaatttgtgtgggtgggtttgccaacaaatgtttatactgctttttcggattatgtttatatttccgcggagctaagctatttgatgatggttattcatggctgtatttcaggtgttttatggagattgtctcgaaattacatatctgtgaaaatattttattcatattttatttataccttttctttcattttttatcatgaaagcttcatgactaatattagcagttggatacaaataactctttgtatcatagtattctctcaatttcttaaggcccagaaaataaagtgaaaatgtattttttgaatgatttaaatttctctagacgcgatgctggatttaaagcaagaaaagatgcactggacattgcttcagattatgaaaacatttctgttgttaacattcctctatggggtggagtagtccagagaattattagttctgttaagcttagtacatttctctgcggtcttgaaaataaagatgttttaattttcaatttcccgatggccaaaccattttggcatatattgtcattctttcaccgccttctaaaatttagaatagtacctctgattcatgatattgatgaattaagaggaggagggggtagtgattctgtgcggcttgctacctgtgatatggtcataagtcacaatccacaaatgacaaagtaccttagtaaatatatgtctcaggataaaatcaaagacataaaaatatttgattacctcgtctcatctgatgtggagcatcgagatgttacggataagcaacgaggggtcatatatgctggcaacctttctaggcataaatgttctttcatatatactgaaggatgcgattttactctctttggtgtcaactatgaaaataaagataatcctaaatatcttggaagttttgatgctcaatctccggaaaagattaacctcccaggcatgcaatttggactcatttgggatggagattctgtcgaaacctgtagtggtgcctttggcgactatttaaagtttaataaccctcataagacatctctttatctttcaatggaacttccagtatttatatgggataaagccgcccttgcggatttcattgtagataatagaataggatatgcagtgggatcaatcaaagaaatgcaagagattgttgactccatgacaatagaaacttataagcaaattagtgagaatacaaaaattatttctcagaaaattcgaacaggaagttacttcagggatgttcttgaagaggtgatcgatgatcttaaaactcgctaaacgatatggtctctgtggttttattcggcttgttagagatgtcttattgactcgtgtattttaccggaactgtagaattattcgatttccctgctatattcgcaatgatggtagcattaattttggtgaaaatttcacaagtggagtcggtctcaggctggatgcatttggacgtggcgtgatttttttttccgataatgtgcaagttaacgactatgttcatatcgcctcaattgagagcgttacgataggtcgggatacgcttattgcaagtaaagtatttattaccgatcataatcacggttcctttaagcactctgatccaatgagttcgccaaatatacctccagacatgcgcacgttggaatcttcagctgttgtaattggccagagggtttggttgggtgagaatgtgacggttttgcctggaacaattattggtaatggagtcgtagtcggcgccaattctgttgttagaggttctattcccgaaaatactgtcattgcgggagtaccagcaaaaatcataaagaaatacaatcatgagaccaaattatgggaaaaagcatagtcgttgtttctgcggtcaattttaccactggcggtccatttaccattttgaaaaaatttttggcagcaactaataataaagaaaatgtcagttttatcgcattagtccattctgctaaagagttaaaagaaagttatccatgggttaaattcattgagtttcctgaggttaaagggtcgtggctaaaacgtttgcactttgaatatgtagtttgtaaaaaactttcaaaagagctgaatgctacgcattggatttgtctgcatgatattacggccaatgtcgtcactaaaaaaagatatgtgtattgtcataaccctgcccctttttataaaggaattttattccgtgaaattcttatggagcctagctttttcttatttaaaatgctatacgggctgatatataaaataaacattaaaaaaaatactgcagtgtttgttcaacaattctggatgaaagaaaaatttatcaagaaatattctataaataacatcattgtcagtcggccagaaattaaattatctgataaaagccaacttactgatgatgattctcaatttaagaataacccttctgagttgacaatattttaccctgctgttccacgagtatttaaaaattacgagcttattattagtgcagcaaggaaattgaaagaacaatccaatattaaatttctgcttactatcagtggtacagaaaatgcgtatgcaaaatatattatcagtcttgcagaaggactggataatgttcatttcctcgggtacttggataaagaaaaaatcgatcattgttataatatttcagatatagtttgttttccctctaggttagaaacatggggattgccgttgtctgaggctaaagagcgaggtaagtgggtattagcatcagatttcccatttactagagaaactcttggtagttatgaaaagaaagctttttttgattctaataacgatgacatgttagttaaacttattattgacttcaaaaaaggtaacctcaaaaaagatatctctgatgcaaatttcatttatcgtaatgaaaatgtattagttgggtttgatgaactagttaattttattactgaagaacattgaaatggtatatataataatcgtttcccacggacatgaagactacatcaaaaaattactcgaaaatcttaatgctgacgatgagcactacaagattatcgtacgcgacaacaaagactctctattattgaaacaaatatgccagcattatgcaggcctggactatattagtggaggtgtatacggctttggtcataataataatattgcggtggcgtatgtaaaggaaaaatatagacccgcagatgatgattacattttgtttttgaatcccgatatcatcatgaagcatgatgatttgctgacatatattaaatatgtcgaaagtaagcgttatgcttttagtacattatgcctgttccgagatgaagcgaaatctttacatgattattccgtaagaaaatttcctgtgctttctgattttattgtgtcatttatgttagggattaataaaacaaaaattcctaaagaaagtatctattctgatacggttgttgattggtgcgcaggatcatttatgctggtacgtttttcagattttgtgcgtgtaaatggcttcgatcaaggttactttatgtactgtgaagatattgacctgtgcttgaggcttagcctggctggtgtcagacttcattatgttcccgcttttcatgcgatacattatgctcatcatgacaatcgaagttttttttcaaaagccttcagatggcacttaaaaagtacttttagatatttagccagaaaacgtattttatcaaatcgcaactttgatcgaatttcatcagtttttcacccgtaagagctcggtacccgggcctagggtgtaggctggagctgcttcgaagttcctatactttctagagaataggaacttcggaataggaactaaggaggatattcatatccgtcgacggcggccgccctgcaggcatgcaagcttgatccatatggatcgctagcttaattaaataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtacgggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtacgggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcaaaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 17 - DNA, 13228 bp, synthetic construct
  misc_feature 1..13228: note = example O18A rfb locus nucleotide sequence - O18A-EPA production strain BVEC-L-00559
  source 1..13228: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagtgaaaatacttgttactggtggcgcaggatttattggttcagctgtagttcgtcacattataaataatacgcaggatagtgttgttaatgtcgataaattaacgtacgccggaaaccgggaatcacttgctgatgtttctgattctgaacgctatgtttttgaacatgcggatatttgcgatgcacctgcaatggcacggatttttgctcagcatcagccggatgcagtgatgcacctggctgctgaaagccatgttgaccgttcaattacaggccctgcggcatttattgaaaccaatattgttggtacttatgtccttttggaagccgctcgcaattactggtctgctcttgatagcgacaagaaaaatagcttccgttttcatcatatttctactgacgaagtctatggtgatttgcctcatccagatgaagtaaataatacagaagaattacccttatttactgagacgacagcttacgcgccaagcagcccttattccgcatccaaagcatccagcgatcatttagtccgcgcgtggaaacgtacatatggtttaccgacaattgtgactaattgctcgaacaactatggtccttatcatttcccggaaaagcttattccactggttattcttaatgcactggaaggtaaggcattacctatttatggcaaaggagatcagatccgcgactggttgtatgttgaagatcatgcgcgtgcgttatataccgtcgtaaccgaaggtaaagcgggtgaaacttataacattggtgggcacaacgaaaagaaaaacatcgatgtagtgctcactatttgtgatttgctggatgagattgtaccgaaagagaaatcttatcgtgagcaaatcacttatgttgctgatcgtccgggacacgatcgccgctatgctattgatgctgagaagattggtcgcgcattgggatggaaaccacaggaaacgtttgagagcgggattcgtaaaacggtggaatggtacctgtccaatacaaaatgggttgataatgtgaaaagtggtgcctatcaatcgtggattgaacagaactatgagggccgccagtaatgaatatcctcctttttggcaaaacagggcaggtaggttgggaactacagcgtgctctggcacctttgggtaatttgattgcttttgatgttcactctactgattattgcggtgattttagtaatcctgaaggtgtagctgaaaccgtaagaagcattcggccggatattattgtcaatgcagccgctcacaccgcagtagacaaagcagaatcagaaccggagtttgcacaattaattaacgcaacaagtgtcgaagcgattgcgaaagcagcaaatgaagttggagcctgggttatccattactcgactgattacgtcttccctggaaatggcgatatgccatggctggagacggatgcaaccgcaccactaaatgtttacggtgaaaccaagttagccggagaaaaagcgttacaggaatattgcgcgaagcatcttattttccggaccagctgggtctatgcaggaaaaggaaataacttcgccaaaacgatgttacgtctggcaaaagagcgtgaagaattagcggttattaacgatcagtttggtgcgccaacaggtgctgaactgctggctgattgtacagcacatgccattcgtgtcgcactgaataaaccggatgtcgcaggcttgtaccatttggtagccagtggtaccacaacctggtacgattatgctgcgctggtttttgaagaggcgcgcaaagcaggcattccccttgcactcaacaagctcaacgcagtaccaacaacagcctatcctacaccagctcgtcgtccacataactctcgccttaatacagaaaaatttcagcagaactttgcgcttgtcttgcctgactggcaggttggcgtgaaacgaatgctcaatgaattatttacgactacagcaatttaatagtttttgcatcttgttcgtgatggtggagcaagatgaattaaaaggaatgatgaaatgaaaatgcgtaaaggtattattttagcgggtggttctggtacacgtctttatcctgtgactatggctgtcagtaaacagctattacctatttatgataaaccgatgatctattacccgctctctacactgatgttggcgggtattcgcgatattttgattatcagtacacctcaggatactcctcgttttcaacaattgctgggtgacggtagccagtggggcctgaatcttcagtacaaagtgcaacctagcccagatggcctcgcgcaggcatttatcatcggtgaagagtttattggtggtgatgattgtgctttggttcttggtgataatatcttttacggtcacgatctgccgaagctaatggaggccgctgttaacaaagaaagtggtgcaacggtatttgcctatcacgttaatgatccagaacgctatggtgtcgttgagtttgataaaaacggtacggcaatcagtctggaagaaaaaccgttagaaccaaagagtaattacgccgttacaggtctgtacttttatgataacgacgtggttcagatggcgaaaaacttgaagccgtctgcacgtggtgagttagaaattacagatattaaccgtatttatcttgagcagggacgtctgtctgtcgcgatgatggggcgtggctacgcgtggctggacacggggactcatcagagtctgatagaagcaagtaattttattgcgacaattgaagagcgccagggattgaaggtttcctgtcctgaagagattgcatttcgtaaaggttttattgatgttgagcaagtaagaaaattagctgtaccactaataaagaataattatgggcagtatctttataaaatgacgaaggattcaaattaatgaatgtgattagaactgaaattgaagatgtgctaattctggagccaagagtatttggtgatgatagaggtttcttttatgagagctttaatcaatcagcatttgaacatattctaggctatccggtcagctttgttcaagacaatcactcacgttcatcaaaaaatgtactcagaggccttcactttcaacgcggcgagtacgcacaagataaacttgtacgctgcactcatggagcagtttttgatgttgctgttgatattcgacccaattcggtatcctttggtaaatgggttggtgttctgctttcagctgataataagcagcagttgtggataccaaaagggtttgctcatggctttttggttctgtctgatatcgctgaatttcaatataaaactacaaactattatcatcctgaaagcgattgtggaatatgttggaatgatgaacgcattgcaattgattggccccaaacatcagggttaatcctttcgccaaaagatgaaaggctctttacgttagatgagcttatcagattaaaattaattgcatgaggccggccttaaggaggactagtcccggcgcgccatgagtttaatcaaaaacagtttttggaacctttgcgggtatgtacttccagctattgtgacactaccagctttgggtattatggggcgaaaattaggcccagaattatttggtgtattcactttggcattagctgttgtgggttatgcaagcatttttgatgcaggccttactcgcgcagtgatacgagaagtcgcaattgaaaaagataatgaagaaaataagttgaaaattatttcttcagcgacagttgtaattatttatttgagtttggccgcctcactcttattatttttttttagtggtcatatcgcattgctactgaacattagtgagactttttttcataatgtaagtgtctcgcttaaaattctcgcagcatccataccattatttttgattactcaaatatggttgtcaattttagaaggtgaagaaagatttggtttacttaatatctacaaatcaattacgggagtgatattagcaatctcaccggcattatttatacttattaaaccctctttgatgtatgcgataataggcttagttctagcaaggtttttatgttttattttggcttttataatttgtcacgataaagtgcttaaagctaaactaacaatcgatataccaacaattaaaagattgtttatgttcggtggttggattacagtaagtaatatcatcagccctgtgctatcatattttgataggtttattgtttcaaatcaacttggggctgctaatgttgctttttatactgcaccatcagaaattatttctcggcttagtataattccaggtgcgttttcaagagccttatttccaagattagctaatgcaaataattccgctgaaagatataaaacgaaaagattaattacaatttcacttttaataatcatcacccctattttttgtattggcgtgttattttcagagaagataatggttttatggatgggggcatcattttttggtgagcctggtttggtattatcaatattactgattggctttatttttaatggattggcacaagtaccatttgccagtattcaatcccgaggtcatgctaagataactgcatttgttcatctcttagagttgtttccttatttattacttttattttacctcataaaagcacatggggttgttggcgcgggtattgcgtggtcagtgaggatgatagtagattatatagcattaagtcttttggacggtaagtatattaataaataaaattcaaaatgcaagttaataactcatggctttatttgggtaggtgacaatttataatgatatatatattaactttaactcttcttctagttatagccataatgttttctcttctcggcacaaaaagtaggatcacatctccattacctttgcattttttaccatggttactaactttaattgtcgggataagtaattacgatcaattttacgagtttaatgaaagaagcttttactctttgttgatttggtttacagttatttttatattttatttcataggggaactggttaattataaacgtgaaaatataaatgtttattatggtctttcacatattaaatatgaatgtaaaaaatattggatcattgtcatcccaatttcattatataccattttcgaaatatatatggttggtatggggggagcagatggattctttctcaatttacgtcttgcaaatacattggagggctatacgggtaaaaaatttatcttaatgcctgctgtatatcctctaatgatggctatgttcgcaattgtttgtctaacaaaaacttccaaattaaataaatactccatttatttctggatgtttttgtattgtattggcacaatgggaaaattttcaatattaacgccaatattgacatatttaattatttatgacttcaaacatagattaaaagtaaaaaaaacaataaagtttacattgttgataattatattagctttaactttgcattttacacgtatggctgagaatgaccactcaacatttttatctattttagggctctatatttattcaccaataattgctttaggccagttgaatgaagtaaatagtagtcattttggtgagtatacgtttagattcatatatgctataactaataaaattggccttattaaagaattgccagtaaatactattcttgactattcatacgttcctgtaccaacaaatgtatatactgcacttcaaccattttaccaggattttggttatactggcatcatatttggagcagtattatacggactaatatatgtgagtttatacacggccggtgttcgtggaaataatacacaggcattactgatttacgcattgttttcagttagcagtgcaacggctttcttcgctgaaacgctagtaacgaatttagctggaaatgtgatgttagtattatgtaccatcttactatggcgatttacagtaatatgcaaaccagtacagtaaccattctaatggccacctacaatggcgaggccttcatcaaaaatcagattttgtcactacaacaacaaacattttctaactggcggttatttattcaggatgatgggtctacagacaatactatatctataataaaaaacttccaaaaatctgactccagaattcggctagttgatgataatttgaaaggtcaaggtgcaggaaaaaattttttatcgctgataaagtacagcgagacagattatacaatttattgtgaccaagatgatatttggttagaaaacaaaatatttgaattagtaaagtatgcaaatgaaattaaattgaatgtatcagatgcgccttcgctagtttatgctgatggctatgcttatatggatggtgagggtacaatcgatttttctgggatatctaacaatcatgctgatcaattaaaggattttcttttttttaatggtggataccaaggatgttctattatgttcaatcgtgcaatgaccaaatttcttctgaattatcgaggatttgtatatctacatgacgatatcacaacattagctgcatacgctcttggtaaagtttattttctcccgaaataccttatgttatatagacagcacacgaatgcggtaactggtatcaaaacattccgcaatggattgacttctaaatttaaatcaccagtaaactatcttttatcacgaaaacattatcaggtaaaaaaatctttttttgaatgtaacagctctatcttatcagagacgaataaaaaagtttttttggattttatttcattttgtgaatcaaataataaatttacagatttttttaagttatggcgaggtgggtttagattaaataacagtagaactaaattattattaaaattcttaatacggagaaaatttagcgaatgatttcaatacttacacctacttttaatcggcaacatactttatcaaggctattcaattctcttatattacaaactgataaagattttgagtggataataattgatgatggtagtatagatgcaacagcggtacttgtagaagattttagaaaaaaatgtgattttgacttgatttattgctatcaggaaaataatggtaagcccatggctttaaacgctggtgttaaagcttgtagaggcgattatatctttattgttgacagtgatgatgcactaactcccgatgccataaaattaattaaagaatcaatacatgattgcttatctgagaaggaaagtttcagcggagtcggttttagaaaagcatatataaaaggggggattattggtaatgatttaaataattcttcagaacatatatactatttaaatgcgactgagattagcaatttaataaatggtgatgttgcatattgttttaaaaaagaaagtttggtaaaaaatccattcccccgtatagaagatgaaaaatttgttccagaattatatatttggaataaaataactgacaaggcgaagattcgatttaacataagcaaagttatatatctttgtgagtatcttgatgatggtctttctaaaaatttccataaccagcttaaaaaatacccaaaggggtttaagatttattacaaagatcaaagaaaacgagagaaaacttatataaaaaaaacaaagatgctaattagatatttgcaatgttgttattatgagaaaataaaatgaaaatactatttgtcattacaggtttaggccttggaggtgctgagaagcaggtttgtcttttagctgataaattaagtttaagcgggcaccatgtaaagattatttcacttggacatatgtctaataataaagtctttcctagcgaaaataatgttaatgtcattaatgtaaatatgtcaaaaaacatttctggagttataaaaggttgtgtcagaattagagatgttatagctaatttcaaaccagacattgtacacagtcatatgtttcatgcaaacattatcactagattgtctgtaattggaatcaaaaacagacctggtattatatcaactgcacataataaaaatgaaggtgggtatttcagaatgctcacatatagaataaccgattgtttaagtgattgttgtacaaatgttagcaaagaagcagtggatgagtttttacggataaaagcctttaatcccgctaaagcaattactatgtataatgggatagataccaataaatttaaatttgatttattggcaaggagggaaattcgagacggtattaatataaaaaatgatgatatattattacttgctgcaggtcgtttaacgttagctaaagattatcctaatttattgaatgcaatgactctgcttcctgaacactttaaacttattattattggtgatggtgaattgcgtgacgaaattaatatgcttataaaaaaattgcaattatctaatagggtgtccttgttgggagttaaaaaaaatattgctccctatttttctgcatgtgatatttttgttctctcttctcgttgggaaggatttggattagtcgtggcagaagctatgtcatgtgagcgaattgttgttggcacggattcagggggagtaagagaagttattggtgacgatgattttcttgtacccatatctgattcaacacaacttgcaagcaaaattgaaaaattgtctttgagccagatacgtgatcacattggttttcggaatcgtgagcgtattttaaaaaatttctcaatagatactattattatgcagtggcaagaactctatggaactataatttgctcaaaacatgaaaggtagatttatatttggaacgtgtcttttgtttgaatttaattcaatctcaattgagatttttgtatttcaaaaataccatcatagctaacgatgattggtatttattttaagatgctttctataaatatattgacgtttttaatgcgccgaaacgattgggctgggaacagagaagtaaaactgttttgagaatgaagagtttttgagatgtttatggatattaaaaattgatccagtgaattaattatttataataaatcaagatttaatgttaataaatgataatcttttctgacactcatattaattatgagtggtacgtttggtaaacggtaaactattatatgacagctagaacaactaaagttttgcacttacaattactcccactcttaagtggcgttcaaagggtaacattaaacgaaattagtgcgttatatactgattatgattatacactagtttgctcaaaaaaaggtccactaacaaaagcattgctggaatatgatgtcgattgtcattgtatccccgaacttacgagagaaattaccgtaaagaatgattttaaagcattgttcaagctttataagttcataaaaaaagaaaaatttgacattgtgcatacacattcttcaaaaacaggtattttggggcgagttgctgccaaattagcacgtgttggaaaggtgatccacactgtacatggtttttcttttccagccgcatctagtaaaaaaagttattacctttattttttcatggaatggatagcaaagttctttacggataagttaatcgtcttgaatgtagatgatgaatatatagcaataaacaaattaaaattcaagcgggataaagtttttttaattcctaatggagtagacactgataagttttctcctttagaaaataaaatttatagtagcaccttgaatctagtaatggttggtagattatccaagcaaaaagatcctgagacattattgcttgctgttgaaaaactgctgaatgaaaatgttaatgttaagctgacacttgtaggagatggtgaactaaaagaacagttagaaagcaggttcaaacggcaagatggacgtataatttttcatggatggtcagataacattgttaatattttaaaagttaatgatctttttatattaccttctctttgggagggtatgccattagcaattttagaagcattgagctgtggacttccatgtatagtcactaatattccaggtaataatagcttaatagaagatggctataatggttgtttgtttgaaattagagattgtcagttattatctcaaaaaatcatgtcatatgttggtaagccagaactgattgcacagcaatctaccaatgcacgatcatttattctgaaaaattatggattagttaaaagaaataataaggtcagacagctatatgataattaagagctcggtacccgggcctagggtgtaggctggagctgcttcgaagttcctatactttctagagaataggaacttcggaataggaactaaggaggatattcatatccgtcgacggcggccgccctgcaggcatgcaagcttgatccatatggatcgctagcttaattaaataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtacgggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcaaaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 18 - DNA, 13554 bp, synthetic construct
  misc_feature 1..13554: note = example O25B rfb locus nucleotide sequence - O25B-EPA production strain stGVXN4459
  source 1..13554: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcagtgaagatacttgttactggtggcgcaggatttattggttctgctgttgttcgtcacataataaataatacgcaagatagtgttgttaatgtcgataaattaacatacgccggaaacctggaatcacttgcagatgtttctgattctgaacgctatttctttgaacatgcggatatttgtgatgcagctgcaatggcacggatttttgctcagcatcagccggatgcagtgatgcacctggcagctgaaagccatgttgaccgttcaattacaggccctgcggcatttattgaaaccaatattgtgggtacttatgtccttttagaagcggctcggaattattggtctggtctggatgatgaaaagaaaaaaaacttccgttttcatcatatttctactgatgaggtgtatggtgacttaccccatccggatgaagtaaatagcaatgaaacgttgccgctatttacggaaacgacagcatacgcgccaagtagtccatattctgcttctaaagcttccagcgatcatttggttcgcgcatggaaacgtacttatggtttaccgaccattgtgactaattgctcgaacaactatggtccttatcatttcccggaaaagcttattccactggttattcttaattcactggaaggtaaggcattacctatttatggcaaaggagatcagatccgcgactggttgtatgtagaggatcatgctcgagcgttatataccgtcgtaaccgaaggtaaagcgggcgaaacttataacattggtggacacaacgaaaagaaaaacatcgacgtagtgttcactatttgtgatttgttggatgagatagtcccgaaagagaaatcttaccgcgagcaaattacttatgttaccgatcgtccgggacacgatcgccgttatgcgattgatgctgagaagattggtcgcgaattgggatggaaaccacaggaaacgtttgagagtgggattcgtaaaacggtggaatggtacctgtccaatacaaaatgggttgataatgtgaaaagtggtgcctatcaatcgtggattgaacagaactatgagggccgccagtaatgaatatcctcctttttggcaaaacagggcaggtaggttgggaactacagcgtgctctggcacctctgggtaatttgattgctcttgatgttcactccactgattactgtggtgattttagtaatcctgaaggtgtagctgaaaccgtaagaagcattcggcctgatattattgtcaacgcagccgctcacaccgcagtagacaaagcagaatcagaaccgaagtttgcacaattactgaacgcgacgagtgtcgaagcgatcgcgaaagcagccaatgaagtcggcgcctgggttattcactactctactgactacgtatttccggggaccggtgaaataccatggcaggaggaggatgcaaccgcaccgctaaatgtttacggtgaaaccaagttagcgggagaaaaagcattacaagagcattgtgcgaagcaccttattttccggaccagctgggtctatgcaggtaaaggaaataacttcgccaaaacaatgttgcgtctggcaaaagagcgtgaagaattagccgttattaatgatcagtttggtgcgccaactggcgcagagttactggctgattgtacggcacatgctattcgtgtggcactgaataaaccggaagtcgcaggcttgtaccatctggtagctagtggtaccacaacgtggcacgattatgctgcgctggtttttgaagaggcgcgcaaagcaggcattccccttgcactcaacaagctcaacgcagtaccaacaacagcctatcctacaccagctcgtcgtccacataactctcgccttaatacagaaaaatttcagcagaactttgcgcttgtcttgcctgactggcaggttggcgtgaaacgaatgcttaacgaattatttacgactacagcaatttaatagtttttgcatcttgttcgtaatggtggagcaagatgtattaaaaggaatgatgaaatgaaaacgcgtaaaggtattattttggcgggtggttctggtactcgtctttatcctgtgacgatggccgtcagtaaacagctgttaccgatttatgataaaccgatgatctattacccgctctctacactgatgttagcgggtattcgcgatattctgattatcagtacaccacaggatactcctcgttttcaacaactgctgggtgacgggagccagtggggcctgaatcttcagtacaaagtgcaaccgagtccggatggtcttgcgcaggcgtttattatcggtgaagagtttattggtggtgatgattgtgctttggtacttggtgataatatcttctacggccacgacctgccgaagttaatggacgtagctgttaacaaagaaagtggtgcaacggtatttgcctatcacgttaatgatcctgaacgttatggtgtcgtggagtttgataataacggtactgcaattagcctggaagaaaaaccgctggaaccaaaaagtaactatgcggttactgggctttatttctatgacaatgacgttgtggaaatggcgaaaaaccttaagccttctgcccgaggtgaactggaaattaccgatattaaccgtatttatatggaacaaggacgtttgtctgtcgctatgatggggcgtggctatgcatggctggatacagggacgcatcaaagtcttattgaagcaagcaacttcattgccaccattgaagagcgccagggactaaaggtttcctgtccggaagaaattgcttatcgtaaagggtttattgatgctgagcaggtaaaagtattagccgaaccgttgaagaaaaatgcttatggtcagtatctgctcaaaatgattaaaggttattaataagatgaacgtaattaaaactgaaattcctgatgtgctgatttttgaaccaaaagtttttggggatgaacgtggcttcttttttgagagttttaatcagaggatttttgaagaagcagtaggtcgtaaggttgagtttgttcaggataaccattctaagtccagtaaaggtgttttacgtggtcttcattatcagttagaaccttatgctcaaggaaaactggtgcgctgtgttgttggcgaggtttttgatgttgcggttgatattcgtaaatcgtcacctacatttgggaaatgggttggggtgaatttgtctgctgagaataagcgtcagttgtggattcctgagggatttgcacatggttttttggtgctgagtgatttagcagaagttttatataaaacgaatcaatattatgctccatcacatgaaaaaaatattatatggaatgacctcttgcttaatattaaatggccgagcacagcactgatcactctgtctgataaggatgcaaatggggaaagatttgaactaagtgagttttgaaatgtctctcttaaaacatagtatatggaatgttgcgggctactttataccaacattaattgcaattcccgcctttggattaattgcgaggaaaattggtgtagaactatttggtttgtatacgttagcaatgatttttatagggtatgcaagtatatttgatgctgggttaacaagagctgttgtgcgtgaaatagcattactaaaaaacagagtggacgattgtaatacgataatagtaacttctattatcgctgtgatatttttagggtttatcggaggcgggggagtgtttctgcttaaaggcgatattattgaactgttaaatatctcaccaatatattacgccgattcgataaagtctctagtattattatcatctctgatacctgtattcttagtcacgcaaatactattagcagagcttgagggtcgggaatattttgggattctaaatatacaaaaaagtgtagggaattctttaattgcagggttacctgcattatttgttttaattaatcaaacgcttttttctgcaattattggtgtagcgattgcaagagttatatgcttgtggttaagctacattatgagcagggaaagaataactatcgatatctcatttttttcaataactgttttaaagcggttatttagatatggcgggtgggtaactataagtaacataatatctcctatattagcgagtatggatagatttattctatcccatatccagggagcatcaaaaatatcattctatacagtccctaatgagctggtaactaggcttggaatagttccaggctctcttgggaaagctgtttttccaaaattaagtcatgcaaggaattttacagcgtcatatgcagagcaaaaaaaagcttatatattaatgactgtcattgtaatgcctttggttttatttgtatattattacgcaaagtttattttaacattgtggatgggggctgagtatgcagggatttcggtcgaaatattacggattatgcttatagggtatatttttaactgttattcacaaatctcttttgccaacatacaggcctttggaaaagcaaaatacactgcatacatccatatgatggaatttattccttatttgataatgttatatataatttcaaaggaatatggggttattggtgttgcgtggttatggacaattcgagtaataattgattttttgatgcttttatatatgagttatcgttgtaataatcttatgaaaaaagggtagcctgatgatatatattgtggtattaaattggaatggggctatagataccattaattgtgttaaaagtttaatggatttaaatgttagcgattataaaattatcattgttgataactgttctatggataactcatatgatactataaaagaaaatcttaattcattatatattgctgataaaagtatcattgaggtgaagtatgaggatagaaataaatataaaaccttagaaaacgataaaatcatattaatacaatctccgcaaaataatgggtacgcaagtggtaataatattggcatagagttcgctcttaatcaggagaatatgaaatacgtctgggttctgaataatgatactgaagtggataaagaggctttaactcatttaattagtaaatgtgattcagataaaagtatagggatttgcggttctcgtttagtctattttgccgacagagagatgcagcaaggactaggtggggtgcataacaaatggttatgcactacaaaaaattatgaaatgggaagattagtttccaaaaaatatgatgatgaagtcattagtaatgatatagattatataattggcgcatcgatgtttttctctagagaatgtttggaaacagttggattgatgaatgaagaatattttttatactatgaagagttagatatttgcctcagagcaaaagcaaagaactttaaattaggtatttgctcagaaagtttggtttatcataaaataggtgcaagtactgatgggggaaagagcatgatggctgatctttgctcaataaaaaataggctggtcattacagaaaggttttatccccaatattattggacggtatggttgtcactttttgttgtagcatttaaccgtgctagaagaggtgagtttaataagatgaaaagatgtttgaatgttatgtttaacttcaaacgaaacaaaggtagcaaatgccattagaatatgcacttaatcatggtgttaataaatctatagtttgatatgttattaaagggtatttaatgaaagtggcttttttatctgcttatgatccactatctacatccagttggtctggcacaccttattatatgctaaaggcattatcgaagagaaatatttccattgaaatattaggaccggtaaatagctatatgatatacatgttaaaagtatataaattaatattaaggtgtttcggaaaagaatatgattatagtcattcgaagttgctttccaggtattacggtagaatattcggtaggaaattaaaaaaaattgatggtttggattttattatcgcacctgcaggttcctcacaaattgcttttttaaaaacaaccataccaataatatatctatcggatacaacatatgatcaattaaaaagctattatccgaatttaaataaaaaaacaattataaatgatgaggatgcaagtttaatcgaacgcaaggctattgaaaaagcaacagtagtatctttcccatctaaatgggcaatggatttttgcaggaattattacagattagattttgataaattagttgaaataccatggggggctaatttatttgatgatattcactttgctaataaaaatataattcaaaagaatagttatacttgtcttttcttgggagttgattgggaaagaaaaggtgggaaaacagccttgaaagcaattgaatatgtaaggcagttatatgggatcgatgttagactaaaaatttgtggatgtactccgaatcaaaagattttacctacttgggttgaattaattgataaagtagataaaaataacgttgacgaatatcagaaattcatcgatgtgttatctaacgctgatatacttcttttaccaaccattgctgaatgttatggaatggtattttgtgaagctgctgcttttggattgcctgttgtcgctacagatacaggtggagtcagttctatagttatcaacgaaaggacggggatattaattaaagacccgttagactataagcactttggaaatgcaattcataaaataattagttccgtagagacttatcaaaactactcccaaaacgcaagaattagatataataatatattgcattgggacaattgggctaaaaagataattgagattatgtatgagcataagaatagaagaatcaaatagcacaaaaagaattatatgtttatttatactttttcttgttttccctgattttttgttttatacattaggggttgataattttagcatttcaacgataatctcaattacattgctttttgtttttttaagagctaaaaatatttgcaaagataattttctaataatagtagcgttattcatattgttgtgttttaactgtttgttaagtatgctatttaatattgaacaggctttaacatttaaagttgtactttcaatatatagcatcttaataatggcatacgtctcctcttgttatgcacagacgttgtggttatgttctgaagaaatacttaagagatccgtcttttatttgttcgcatttctttgccttattggcattataagtattcttttacagaagactgagattatacatgataaaagtatgattctttttcctgaaccatcagcatttgcattggtttttatacctatcttttcattttgtttatactatacaagagggggggggctactattgctctatatattatctttgggtattgcgttaggtatccagaatttaacaatgttggtaggcattgtgattagtgtttttgtgatgaaaaaaataactataaggcaaactattgttatacttttgggggcatggattttttccatgatattaagtgatttagacatttcttactatacatcgcggcttgattttaaaaatactacgaacctatcagtgcttgtatatctttcaggaattgaaagagctttcttgaattttattacaagttatggtcttggtattggttttcaacaaatgggagtgaatggggagataggaatatatcaacaaattttagctgaacttgatgcccctatgttaaatatatacgatggctcatttatttcttctaagttaatatctgagtttggggttattggtgcattaatgtgtattttctattttttttatttttcccgattttatctgcgtttcaaaaaaagtaagagatattcaccgcagtatattttagcatatagcttctacatgtgtttcttcatccctctttttatacgtggtgctggttatataaacccctatgtgtttatgttattttcatcaatatttttgtgcaaatatcacgctaaaaatatcttgatgaaatctaatgtccagatagctatataatagtagattatattatcattatcacgtaaattacatattaatagcatatatgataactaggacataaataatgtgcattaaaaaaaaacttaagttaattaaacgatatggcctttatggtggtcttaggcttcttaaagatatattcttaacaaaatttttattttgttcaaatgttaggattattagatttccatgttatattagaaaagatggaagtgttagttttggaaaaggttttacatcaggtgtaggattacgagttgatgcatttatggatgccgtagtttccattggagaaaatgttcaaattaatgactatgttcacatcgcggctattaataatgtcattattggtagagatacattaatagcaagtaaagtatttattagtgatcataatcatggtattttttctaaatccgatatccatagttcaccaactattattccttcgtctaggccccttgaatctgcacctgtgtatattggagagcgtgtgtggattggcgaaaatgtgacaatattaccaggtgcgtgtataggtaatggtgtagttattggcgcaaacagtgttgttcgtggtgagattcctaataatgtgatcattgctggtgttccagctaaaattgttaaaaaatataactatgagcgtatgcaatgggaaagaatatagttgtaatatcggctgttaattttacaaccggaggcccctttaccgtactaaaaaatgtgcttacagcaactaaagatagagccgaatgtaaatttattgcactggttcatagctctgctgaactaatggaattatttccgtgggttgaatttatagagtatccagaagtcaagtcttcgtgggttaaaagattatatttcgaatatataacttgcaatagattatctaaggtgattaaggcaactcattgggtatgcttacatgatattacagcaaatgttagtgtaccctatagatttgtttattgccacaatcctgcaccgttctataaatatttaagctatcgagatattataggagaacctaaattttatcttttttatcttttttatgggcttttatacaatatcaatataaaaaagaacacagcagtttttgttcagcagcagtggctaaaaaaagaattcgaaaaaaaatataagttaaagaatgttgttgttagtcgccctgaagatatttgcccttttgaaagtgatggtttggtaagaaataataataaaaaggatgtgaggatattttacccagcagtgccccgtatatttaaaaactttgaagttatcatacgtgctgcacaaatattacaagataaaaatattcatttttatcttacttttgatggtactgaaaataagtatgcaaaaagaatatataaattagcttccgaactgaaaaatgtacatttcctcggttaccttaatgcaaccgagatggttaacttttatcaagattcagatattatttgtttcccatcgaaactagaaacgtggggattaccattatcagaagctaaaacatacaaaaaatggatatttgcggcagacttaccttatgctcatgaagttttatataactattcaaaaactagatattttccatttgacgatgagaaaatacttgttcgctacatattagagtacacaagtaaaaatatgcatgaagatataaaaaatagtagggtgaattttaataatgatgcattgactggttttgaacagtttattgaatatatcctcaaggggaactgacgtggtttatattataatcgtttcacatggccatgatgactatatagaaaatcttttattaaatttaaagttgccctctggaagatttaaaataatagttcgtgataacaaaagttcaatggttttaaaaaaaacatgcgaaaaaaattgcgtaacctatttgcatggagggcaatatggatttggacataataataacatagcagtgtcatatataattaataacttcatgattatgaataatgattattttctctttcttaaccccgatgtattcataaccagtgaaagtttgattaattatgttgattatataattagtaatgattataagtttagcacattatgtctttatcgagattttactaaaagcaaacatgattattcaatacggagttttccaactttatatgattttctttgttcttttttattgggggtgaataaaagtaaaattaagaaggaaaatatactttctgatactgtagttgattggtgtgctggctcatttatgcttattcatgctttaagtttcttaaatgtgaatggttttgatcaaaaatattttatgtattgtgaagatattgacctttgtatgcgtttaaaattaagtggagtagatctttactatactccccattttgatgctattcattatgcgcagcatgaaaatagaagaatatttactaaagcatttcgatggcatataaggagtattacgcgctacatattacggaaaccaattctttcttataaaaactatagaaaaattacatccgaactggtaaagtgattaaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 19 - DNA, 15197 bp, synthetic construct
  misc_feature 1..15197: note = example O75 rfb locus nucleotide sequence - O75-EPA production strain stLMTB11737
  source 1..15197: mol_type = other DNA
atgacgaatttaaaagcagttattcctgtagcgggtctcgggatgcatatgttgcctgccactaaggcgatacccaaagagatgctaccaatcgtcgacaagccaatgattcagtacattgttgacgagattgtggctgcagggatcaaagaaatcctcctggtaactcacgcgtccaagaacgcggtcgaaaaccacttcgacacctcttatgagttagaatcactccttgagcagcgcgtgaagcgtcaactgctggcggaagtacagtccatctgtccgccgggcgtgaccattatgaacgtgcgtcagggcgaacctttaggtttaggccactccattttgtgtgcgcgacctgccattggtgacaacccatttgtcgtggtactgccagacgttgtgatcgacgatgccagcgccgacccgctacgttacaaccttgctgccatgattgcacgtttcaacgaaacgggccgcagccaggtgctggcaaaacgtatgccgggtgacctctctgaatactccgtcatccagactaaagagccgctggaccgtgagggtaaagtcagccgcattgttgaatttatcgaaaaaccggatcagccgcagacgctggactcagacatcatggccgtaggtcgctatgtgctttctgccgatatttggccggaactggaacgtactcagcctggtgcatggggacgtattcagctgactgatgctattgccgagctggcgaaaaaacaatccgttgatgcaatgctgatgaccggcgacagttacgactgcggcaaaaaaatgggctatatgcaggcgtttgtgaagtatggcctacgcaacctgaaagaaggggcgaagttccgtaaaggtattgagaagctgttaagcgaataatgaaaatctgaccggatgtaacggttgataagaaaattataacggcagtgaaaattcgcagcaaaagtaatttgttgcgaatcttcctgccgttgttttatataaaccatcagaataacaacgagttagcagtagggttttattcaaagttttccaggattttccttgtttccagagcggattggtaagacaattagcgtttgaatttttcgggtttagcgcgagtgggtaacgctcgtcacatcataggcatgcatgcagtgctctggtagctgtaaagccaggggcggtagcgtgcattaatacctctattaatcaaactgagagccgcttatttcacagcatgctctgaagtaatatggaataaattaagctagcagtgaagatacttgttactggtggcgcaggatttattggttctgctgttgttcgtcacataataaataatacgcaagatagtgttgttaatgtcgataaattaacatacgccggaaacctggaatcgctcgctgaaatttctgattctgaacgttattcatttgagcatgcagatatctgcgatgccgaagcgatggctcgtattttcgcacagcaccagccagacgcggtgatgcacctggcagcagagagccacgttgaccgctcaataactggccctgcggcatttattgaaaccaatattgtgggtacttatgttcttttagaagcggcgcgcaattattggtctggtctggatgatgaaaagaaaaaaaacttccgctttcatcatatttctactgatgaggtgtatggtgacttaccccatccggatgaagtaaatagcaatgaaacgttgccgctatttacggaaatgacagcatacgcgccaagtagtccatattctgcttctaaagcttccagcgatcatttggttcgcgcatggaaacgtacttatggtttaccgaccattgtgactaattgctcgaacaactatggtccttatcatttcccggaaaagcttattccactggttattcttaatgcactggaaggtaaggcattacctatttatggcaaaggagatcagatccgcgactggttgtatgtagaggatcatgctcgagcgttatataccgtcgtaaccgaaggtaaagcgggcgaaacttataacattggtggacacaacgaaaagaaaaacatcgacgtagtgttcactatttgtgatttgttggatgagatagtcccgaaagagaaatcttatcgtgagcaaattacctatgttgctgatcgcccagggcatgatcgccgttatgcaattgatgccgataaaattagccgcgaattgggctggaaaccacaggaaacgtttgagagcgggattcgtaaaactgtggaatggtatctgtccaatacaaaatgggttgataatgtgaaaagtggtgcctatcaatcgtggattgaacagaactatgggggccgccactaatgaatatcctcctttttggcaaaacagggcaggttggttgggaactacagcgtgctctggcacctctgggtaatttgattgctcttgatgttcactccactgattactgtggtgattttagtaaccctgaaggtgtggctgaaaccgttagaagcattcggcctgatattattgtcaacgcagccgctcacaccgcagtagacaaagcagaatcagaaccggagtttgcacaattactgaacgcgacgagtgtcgaagcgatcgcgaaagcagccaatgaagtcggcgcttgggttattcactactctactgactacgtatttccggggaccggtgaaataccatggcaggaggaggatgcaaccgcaccgctaaatgtttacggtgaaaccaagttagcaggagaaaaagcattacaagagcattgtgcgaagcaccttattttccggaccagctgggtctatgcaggtaaaggaaataacttcgccaaaacgatgttgcgtctggcaaaagagcgtgaagaattagccgttattaatgatcagtttggtgcgccaactggcgcagagttgctggctgattgtacggcacatgccattcgtgtggcactgaataaaccggaagtcgcaggtttgtaccatctggtagccagtggtaccacaacctggcacgattatgctgcgctggtttttgaagaggcgcgcaaagcaggcattccccttgcactcaacaagctcaacgcagtaccaacaacagtctatcctacaccagctcgtcgtccacataactctcgccttaatacagaaaaatttcagcagaactttgcgcttgtcttgcctgactggcaggttggtgtgaaacgcatgctcaacgaattatttacgactacagcaatttaatagtttttgcatcttgttcgtgatggtggaacaagatgaattaaaaggaatgatggaatgaatacgcgtaaaggtattattttagcgggtggttctggtacacgtctttatcctgtgactatggctgtcagtaaacagctgttaccgatttatgataaaccgatgatctattacccgctctctacactgatgttggcgggtattcgcgatattttgattatcagcacgccacaggatactcctcgttttcaacaactgctgggtgatgggagccagtgggggctaaatcttcactacaaagtgcaaccgagtccggatggtcttgcgcaggcatttatcatcggtgaagagtttatcggtggtgatgattgtgctttggtacttggtgataatatcttctacggtcacgacctgcctaagttaatggatgccgctgttaacaaagaaagtggtgcaacggtatttgcctatcacgttaatgatcctgaacgctatggtgtcgttgagtttgataaaaacggtactgcaatcagcctggaagaaaaaccgttacaaccaaaaagtaattatgcggtaaccgggctttatttctatgataactacgttgtggaaatggcgaaaaatcttaagccttctgcccgcggtgaactggaaattaccgatattaaccgtatctatatggaacaggggcatttatctgttgccatgatgggacgtggatatgcctggctggacacggggacacatcaaagtcttattgaagcaagcaacttcattgccaccattgaagagcgccagggcttgaaagtttcctgcccggaagaaattgcttaccgtaaagggtttattgatgctgagcaggtgaaagtattagctaaaccgctgaaaaaaaatgcttatggtcagtatctgctaaaaatgattaaaggttattaataaaatgaatgttattaaaacagaaattccagatgtactgatttttgaaccgaaagtttttggtgatgagcgtggtttctttatggaaagctttaatcagaaagttttcgaagaggctgtagggcggaaggttgaatttgttcaggataatcattctaaatcgtgtaaaggtgtacttagaggtttacactttcagcttcctccctttgagcaggcaaaattagtaaggtgtatagttggcgaggtatttgatgttgcagtagacattagacctaattctgaaacatttggttcatgggttggagtaactctttcgtcagaaaataaaaggcagctatggattccagaaggattcgcccatggttttttaactttaagtgatattgcagagtttgtttataaaactaacaactattattctttaaatcatgaaaggggagtcatttggaacgatgaggaaattaacattgcctggccctctcaatcagagaagattctgtcacagaaagatattaatttaccatcatttagatttgttcaaatgtttagcaagtagtgttatctttacactgcacatagtcatcattttttatgctttaagtaaattatattgcacatctataacacaaagcgcaataatatttcgacctgatgaaggtttgtggttatttatctttctaggcgttttttatgactaaaatagttgtggtttctacagctccaatattcccgacaaataatgggtacaaaagttctgtattaggaagaattgatgagttattaaatgaggataatgaggtcgttttgattgaaataaaccttgaaaatgttacggaaaagaaagatgaattaataccaacaagatttaataatattcaaagatatgaagtaaaaaaaatatctagatcatttattgccgagttacaaatattatttgatatcagaactcggtatgaacaattattttcttctgctgacattagagataacataaaaaagataattgatttagaaaaaccttctattattattgctgagtctatatgggcgttgcaagcattgcctattgaaattagtgcgagaatacactgtgttattcatgatgtggcaactgatttctttaaagaaatgtttgtatctcataatgaggttgtacgaaaaattttgttttttaatgattacctaaagttgaaaattactgaagaaaatattatcaaacgtttgagagttgagcaatttatctttctgacagaagaagataaatgttggtataaaacaagatacaatattgatgagggttgttgttccttagcgagcaatcatctttatgtagaaaagattaagagaactatcaatttccaaacccctttcctgcttattcccggtagcattgaattttcacaaaatttttacggcttaaattggtttataaaaaatatatatcctggattaaataggaaaataagaatagttgtaacaggaaaggcatcagataaaaaaataaagatgttaaactgtggagaggaaattacctttacgggagagcttgacttttccacatataataaacttagctcaacatgcttgtgtgttattgcaccgattacaacgggcactggaattaaaataaaaatattagaagctgtacaaaaaggtattcctgtacttacaacaaaatttgcttcaaaaggaatatgttccgatttatgtttttattgcgaggaggatactgacacaaactttgtcaatttaattaacagttttcttgaaacgacattaagagtccaagaatgaatttattgcttttttcagtccttgcgtttggtttaatattggctttggcccataataataaaagtggagatattaacgcatacttaatgttttttctcgtggtcctaatggtattaatatcagggctgcgtatgaatgatagtgattatatcgaatacaggaaaatgtataatgaagtgcctattttatgtgactttagtctcgcatctataagagatatacatggggaggtaggctatctattcttatcatcaatctttaaaactttatgcttgccatttcaattatttcttttttttattgcttttttatcactcctgcttacatatttttcattcagaaaaataagtttaataccgatactatcgttagttttttatttaagccatgcttttatagttagagatttgattcaaattagggcaggattagctgttagcatatcattatattcaataattaaatttaaaggaaataaaagtataattacaggagttttatttgcttctttgattcattctggggcgcttattattgctctttgttatccttttttcaaaaaaaaatacataacattaaaaatgatgttgtttttatttttagtgtcaattattttttcttatttgaatgggcttaatttatcgatacaactcttatctcaatatagtttgcttccaactgcaatttcgaattatgttggttgggaagaatatgattatcgggtgagtatatttactaatccggtttttattaaaggtgtttttttaattgtcttaatgcacaaatatgtactttcagatattaaaaatgagaaaattatagtgctttataacttatatgttttaggtgtattagctatggttgcattgagtgggatggctattctttcaggccgtctttcatcctttctgacactaggtgaaagcattttaattgtatatgctctgttctacaaaagaaatacacctctggcgtttctaattttttcttttttaacaattgtgcaattaggatatgatctatttatttctaatgtgcatcctgagcttactctgattatatttgggtgaatctaagtgaaaaataataaaataggcatacttatctctaaaatacaaaatcttggacctgtgaatgtagtacgaggattgataaaagaaaataaaaaatatgcttttactgttttttgtttaacaaatagcgtagataaaaatatatatgatgagttatgctgtttaggagccaaggttatattaataccagatggtacttggttcagcaaaattttatttgtgagaagttttttaaaggaacatccacataatatcttacattcacatgggatcacggccgatatgttttcttactttctgaatggcgtgaaaatatctactattcacaatagactagatgaggattatatcccattatttggcgcggttaaagggaatgctatatattatcttcatcgttttatattacgaagatttaatcatatcgttgcttgctcagcagcggtccaatcaaaactgaaacaatcgaaagtaaaaactaaaataaccaccatccagaatgggattgatataactaggtttaagacacttgagtctgataaaaaaaaattattgagggaaaaacacggatttgatagtgaaaaaagaatatttatatattgtggctcgttatcattaaggaaaaatattgcttacctcttggaacacttagccatcgaagaaaatgatatatttttaattctaggtgatggtgaactttttagatattgtaaggataaatattctaaagatttacggtatatatttatggggaaagttgaatgccctcttgaatattatcaattatcagatatttttgtttccgcttctttatcggaagggctccccttggcactattagaagctgcctctactgggtgctatttatatgttagcgatatagagccccatagagaaattgcatctctattaggagaggaaaatatttctatgtttaaaattaaggatggatcatataattatttgcaacctaaaataaaaaaagctgactataacgctctttctgacgataaactttacaatatatccgataaaaaaatgtcaaatctttatgacaaactttttgtttctttattagagcagaggcactaatataatgatttatgtttcggtaatttctcatggtcatttcaaaactcttaaggaattaggagcagtatcaaaattaaataatcacagcagaattaaagttatcatcaaagataatttaggagagagcgagcttttggatttttgtcaggaaaacaaaataacttatttaaggtctaaagagaaaaaaggatttggagagaataataatgaagttttttcctctatatcctccttaattactaaggaagatttttttgtggttatgaatcctgatatatatattgagtgctctgatctattagatgtcgtagatgagtgtggttcagcgaatgttaatctagcaacgataaatttatacagggattttgataaaaaaacatatgataactcagtaaggaaatttccctcggcaattgatttttttatgtcatttttatttaagaaaaatgactgtgtagtaaataagaacaaaataacgaaaccaacatatgttgattgggctgcaggttcttttctaatatttaatgccttcttttattcaaaactcaacggattcaacgaaaagtattttatgtattgcgaagatattgatatatgttggcgagctaaaaaacacttcaatacttcagttttatactatccatgctatgcagcaattcatttggcacaatttaacaatcgtaggatttttagtagacatttcatttggcatataaaaagtattatcctttttttattatataaaaatggtatgctgcgttctagtaagttgctttaatgctaatattcttttaagaggtgagaatgatacctgttattttggctggtggttcgggaagtcgcttgtggccactttcacgagaaaagttccccaagcagtttttaaagttgactggcagtttgacaatgttgcagtcaacattgtcacgtcttaataatttaaatgctgatgattcaatagttatatgcaacgaagagcatagatttattgttgcagaacaattaagagagttaggcaaactttcaaataacattattcttgaacccaaaggtcgtaatacagcccctgctataacactcgcagcattagcagcaaaaagaaaattcgctgatgaagatccattgattcttattttagctgcagatcacaacatccaagacgaacatgttttctgtgaggcaattaataaggcgtcatctttagctagttatggaaaactagtgacttttggtatcgttccattcaaacctgaaactgggtatggctatattcgtcgcggtgatgaagtgcctgtagatgagcagcatgcggtggcctttgaagtggcgcagtttgtcgaaaaaccgaatctggaaaccgcgcaggcctatgtggcaagcggcgaatattactggaacagcggtatgttcctgttccgtgccggacgctatctcgaagaactgaaaaagtatcgtccggatattctcgatgcctgtgaaaaagcgatgagcgccgtcgatccggatctcgattttattcgtgtggatgaagaggcgtttctcgcttgtccggaagagtcggtggattacgcggtcatggaatgcacggcagatgccgttgtggtgccgatggatgcgggctggagcgatgtcggttcctggtcttcattatgggagatcagcgcccacaccgccgagggcaacgtttgccacggcgatgtgattaatcacaaaactgaaaacagctatgtgtacgccgaatctggcctggtcaccaccgtcggggtgaaagatttggtggtagtgcagaccaaagatgcagtgctgattgccgaccgtaatgcggtgcaggatgtgaagaaagtggtcgagcagatcaaagctgatggtcgccatgagcatcgggtgcatcgcgaagtgtatcgtccgtggggcaaatatgactctatcgacgcgggcgaccgctaccaggtgaaacgcatcaccgtgaaaccgggcgaaggtttgtcggtacagatgcattatcatcgcgcggaacactgggtggttgtcgcgggaacggcaaaagtcactatcaacggtgatatcaaactgcttggtgaaaacgagtccatttatattccgctgggggcgatgcactgcctggaaaacccggggaaaatagatttagaattaattgaagttcgctctggtgcatatcttgaagaagatgatgttattagatgttatgatcgctatggacgaaagtaatatataataattatttcagaattagaaatgataattataagttttcgtctggataaacaatagatagtatgggttggaaaatatgagttctttaacttgttttaaagcttacgacattcgcgggaaattaggtgaagaactgaatgaagatatcgcctggcgcattggtcgcgcctatggcgaatttctcaaaccgaaaaccattgtgttaggcggtgatgtccgtctcaccagcgaaaccttaaaactggcgctggcaaaaggtttacaggatgcgggcgtcgatgtgctggatattggcatgtccggcaccgaagagatttatttcgccacgttccatctcggcgtggatggcggcattgaagttaccgccagccataatccgatggattacaacggcatgaagctggtgcgcgaaggggctcgcccgatcagcggtgataccggactgcgcgacgtccagcgtctggcagaagctaacgactttcctcccgtcgatgaaaccaaacgcggtcgctatcagcaaatcaatctgcgtgacgcttacgttgatcacctgttcggttatatcaatgtcaaaaaccttacgccgctcaagctggtgatcaactccgggaatggcgcagcgggtccggtggtggacgctatcgaagcccgctttaaagccctcggcgcaccggtggagttaatcaaagtgcataacacgccggacggcaatttccccaacggtattcctaacccgttgctgccggaatgtcgcgacgacacccgcaatgcggtcatcaaacacggcgcggatatgggcattgcctttgatggcgattttgaccgctgtttcctgtttgacgaaaaagggcagtttattgagggctactacattgtcggcctgctggcagaagcgttcctcgaaaaaaatcccggcgcgaagatcatccacgatccacgtctctcctggaacaccattgatgtggtgacggccgcgggcggcacgccggtgatgtcgaaaacaggacacgcctttattaaagaacgtatgcgcaaggaagacgccatctacggtggcgaaatgagcgctcaccattacttccgcgatttcgcttactgtgacagcggcatgatcccgtggctgctggtcgccgaactggtgtgcctgaaaggaaaaacgctgggcgaactggtgcgcgaccggatggcggcgtttccggcaagcggtgagatcaacagaaaactggcgcaccctgttgaggcgattaaccgcgtggaacagcattttagccgtgaggtgctggcggtggatcgcaccgatggcatcagcatgacctttgccgactggcgctttaacctgcgctcttccaacaccgaaccggtggtgcgcctgaatgtggaatctcgcggtgatgttcaggttatggtaatccatactcaagaaatattatcaattttgacgtcataaagaataagccctgacaagttagggcttaattaatatatattttttttgaattggggatttgtggtaagatttttaatatgttatttaatgtggttgaattaatgttgactggaaaataataatgagaacgaaaaaagcattacacaactttaaagttgatttattaattacttttttattggttttgctagggttttatattcgaactgtttttgtttcaaaaatgggaagtgatattactggagtgatgttactattcacacagttgacagcatatctcaatttggcagaattaggtattggaattgcagctgccagcgtattatataaaccgctcagcgagaatgaatacaataaaataacttacataatatctttgctctcagtcatatacaaatatatatttgtgtttgttttgattcttggcgttgttataggtatctgtatttattactttattgattctgtaaaggttgtaaatggcgtttttttatattgggctttgttcgtttttaatacatcgttgacatatagttatgctaaatactccacattattaactgctaatcagcggtactcagcagtaagaaaaattcaaggtggcggaaaagttataataattgtatttcagatattaattttgtgctttacgcaaagtttcatactttatttgttagttgagactttaggtattttttctcaatatttgatttttaaaaaaataattgggaacggaaatcaatatctcagtaatgaggttttacttattgaaagcgataaacttttgataaaaaaagaattaaaaataagaataaaaaatatgttcttccataaaataggtgctgtgcttgtccttaatacagactacctgcttgtatcaaagtttctgacattaagttatgtgacaatttttggcagctatatgatggtatttcagatagtaactgttttgatgtcaagttttgttaatgctattactgcaggaatgggtaattacttaattaataaaagtaatttagaaattaaggaaattacacgtcaattttatgtgatatttatcgcctttgcaacattcatatcactaaatatgttttttcttgttaatgattttatcgcaaaatggataggtgttaattatacattaagtaacaccctagttgcattaatgattgttaacgtattcattagtgttgtcagggtaccttctgatatattaaaaaacgcaagtggacattttggtgatatttattatccattattagaaggtgtgctgaatattacgatatccatcattttggctatcattattggattacctggcattattatagggacaatagtatctaacttaatagtaataatgcttgcgaaaccattatatctttactctaagttatttaatcttagaaatccgacgagggtttattttgaatttatttctcggcctatgttatattcattatgtgtgattggggtgagctatttattgcgcgatgaaatatattcatttaaagtaagtacatggttggattttattaacaagctactcttagtctctactcctagcatattggtaatatgtgctattttctctacggatagtgactttagattatttttcagaaaaattatatatgtgattatgaagaaataaaaatttcgaaaatgtattaatcgaaattatgcaacgagctttatttttataaatgatatgtgatcttttcgcgaataggagtaaggatccgtgtaggctggagctgcttcgaagttcctatactttctagagaataggaacttcggaataggaactaaggaggatattcatatggataaagccgtaagcatataagcatggataagctatttatactttaataagtactttgtatacttatttgcgaacattccaggccgcgagcattcagcgcggtgatcacacctgacaggagtatgtaatgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 20 - protein, 535 aa, synthetic construct
  REGION 1..535: note = Example CRM197 sequence
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS